(19) United States
(12) Patent Application Publication
(10) Pub. No.: US 2026/0048130 A1
(43) Pub. Date: Feb. 19, 2026

(54) ALPHA-V BETA-6 (avb6) INTEGRIN LIGANDS FOR EXTRAHEPATIC DELIVERY

(71) Applicant: Alnylam Pharmaceuticals, Inc., Cambridge, MA (US)

(72) Inventors: Jayaprakash K. Nair, Cambridge, MA (US); Joseph David Panarese, Newton, MA (US); Bhaumik Pandya, Bedford, MA (US); Karyn Schmidt, Melrose, MA (US); Kevin Dooley, Melrose, MA (US); Bryan Ingoglia, Waltham, MA (US); Oliver Galloway, Boston, MA (US); Masaaki Nakata, Cambridge, MA (US); Justin Pierson, Wakefield, MA (US); Yesseinia Anglero-Rodriguez, Cambridge, MA (US); Scott Lentini, Braintree, MA (US); Vasant R. Jadhav, Sharon, MA (US)

(21) Appl. No.: 19/235,730

(22) Filed: Jun. 12, 2025

Related U.S. Application Data
(63) Continuation of application No. PCT/US2023/083947, filed on Dec. 14, 2023.
(60) Provisional application No. 63/432,448, filed on Dec. 14, 2022.

(51) Int. Cl. A61K 47/54; A61K 9/00; A61P 25/28

(52) U.S. Cl. CPC A61K 47/545; A61K 9/0019; A61K 9/0043; A61K 9/007; A61P 25/28

(57) ABSTRACT

The present invention provides double stranded ribonucleic acid (dsRNA) agents for inhibiting expression of a target gene, comprising an antisense strand which is complementary to the target gene; a sense strand which is complementary to the antisense strand and forms a double stranded region with the antisense strand; and at least one alpha-v-beta-6 (αvβ6) integrin targeting ligand that mediates delivery to an extrahepatic tissues, e.g., muscle tissue, e.g., skeletal muscle tissue and/or cardiac muscle tissue, or lung tissue conjugated to at least one strand, compositions comprising such dsRNA agents, and methods of use thereof for treating a subject having a disorder that would benefit from reduction in expression of the target gene.

### CROSS-REFERENCE TO RELATED APPLICATIONS

This application is a 35 § U.S.C. 111(a) continuation application which claims the benefit of priority to International Application No. PCT/US2023/083947, filed on Dec. 14, 2023, which, in turn, claims the benefit of priority to U.S. Provisional Application No. 63/432,448, filed on Dec. 14, 2022. The entire contents of each of the foregoing applications are incorporated herein by reference.

### SEQUENCE LISTING

The instant application contains a Sequence Listing which has been submitted electronically in XML format and is hereby incorporated by reference in its entirety. Said XML copy, created on Jun. 4, 2025, is named 121301_21102_SL.xml and is 2,170,587 bytes in size.

### BACKGROUND OF THE INVENTION

Efficient delivery of an RNAi agent to cells in vivo requires specific targeting and substantial protection from the extracellular environment, particularly serum proteins. RNAi-based therapeutics show promising clinical data for treatment of liver-associated disorders. However, RNAi delivery into extra-hepatic tissues remains an obstacle, limiting the use of RNAi-based therapies.

One of the limiting factors is the ability to deliver intact RNAi efficiently to extra-hepatic tissues, such as muscle tissues, e.g., skeletal muscle tissues and/or cardiac muscle tissues, or lung tissue.

Previous work has used delivery reagents such as liposomes, cationic lipids, and nanoparticles forming complexes to aid the intracellular internalization of RNAi agents into extra-hepatic cells. However, only limited success in delivering RNAi agents to extra-hepatic tissues, like muscle tissue, after systemic administration has been reported. For example although cholesterol-conjugated RNAi agents are delivered to muscles after intravenous injection, a high dose (50 mg/kg) is required to achieve sustainable gene silencing. In addition, cholesterol conjugates are highly toxic at high concentrations, limiting their potential for clinical applications.

Thus, systemic delivery of oligonucleotides to muscle tissue remains a challenge and, accordingly, there is a continuing need for new and improved compositions and methods for delivering RNAi agents in vivo.

### SEQUENCE LISTING

This application contains a Sequence Listing which has been submitted electronically in .XML format and is hereby incorporated by reference in its entirety. Said .XML copy, created on Dec. 12, 2023, is named “121301_21120_SL.xml” and is 7,359,147 bytes in size. The sequence listing contained in this .XML file is part of the specification and is hereby incorporated by reference herein in its entirety.

### SUMMARY OF THE INVENTION

The present invention is based, at least in part, on the discovery of alpha-v-beta-6 (αvβ6) integrin binding compounds suitable for conjugating to a cargo molecule to be delivered to a cell (e.g., single- and double-stranded oligonucleotides) and the surprising discovery that conjugating at least one such alpha-v-beta-6 (αvβ6) integrin binding compound to at least one strand of a dsRNA agent, e.g., the sense strand, provides surprisingly efficient in vivo delivery to extrahepatic tissue expressing αvβ6, i.e., muscle tissue, resulting in efficient entry and internalization of the dsRNA agent into extrahepatic tissue, e.g., muscle tissue, e.g., skeletal muscle tissue and/or cardiac muscle tissue, or lung tissue, and surprisingly good inhibition of target gene expression in extrahepatic tissue, e.g., muscle tissue, e.g., skeletal muscle tissue and/or cardiac muscle tissue, or lung tissue.

Accordingly, in one aspect, the present invention provides a double stranded ribonucleic acid (dsRNA) agent for inhibiting expression of a target gene, comprising an antisense strand which is complementary to the target gene; a sense strand which is complementary to the antisense strand and forms a double stranded region with the antisense strand; and at least one alpha-v-beta-6 (αvβ6) integrin targeting ligand that mediates delivery to muscle tissue, e.g., skeletal muscle tissue and/or cardiac muscle tissue, conjugated to at least one strand.

In another aspect, the present invention provides a double stranded ribonucleic acid (dsRNA) agent for inhibiting expression of a target gene, comprising an antisense strand which is complementary to the target gene; a sense strand which is complementary to the antisense strand and forms a double stranded region with the antisense strand; and at least one alpha-v-beta-6 (αvβ6) integrin targeting ligand that mediates delivery to lung tissue conjugated to at least one strand.

Exemplary ligands can be found in U.S. Pat. Nos. 10,023,568, 10,450,312, 10,144,733, 10,487,080, 10,513,517, and 10,000,489, the entire contents of each of which are incorporated herein by reference.

In one embodiment, the ligand is conjugated to the dsRNA agent via a linker comprising a compound of the structure

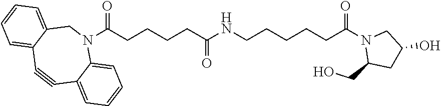 [SMILES: O=C(CCCCC(=O)N1Cc2ccccc2C#Cc2ccccc21)NCCCCCC(=O)N1C[C@H](O)C[C@H]1CO]

In one embodiment, the ligand comprises a compound of the structure

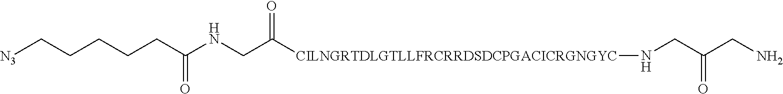

### (SEQ ID NO: 121).

In one embodiment, the ligand comprises a compound of the structure

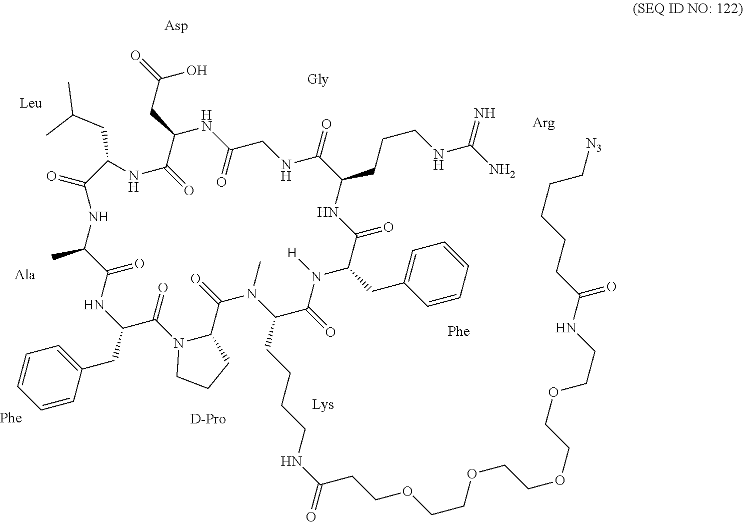 [SMILES: CC(C)C[C@@H]1NC(=O)[C@@H](CC(=O)O)NC(=O)CNC(=O)[C@@H](CCCNC(=N)N)NC(=O)[C@H](Cc2ccccc2)NC(=O)[C@H](CCCCNC(=O)CCOCCOCCOCCOCCNC(=O)CCCCCN=[N+]=[N-])N(C)C(=O)[C@@H]2CCCN2C(=O)[C@H](Cc2ccccc2)NC(=O)[C@@H](C)NC1=O]

(SEQ ID NO: 122) In one embodiment, the ligand comprises a compound of the structure

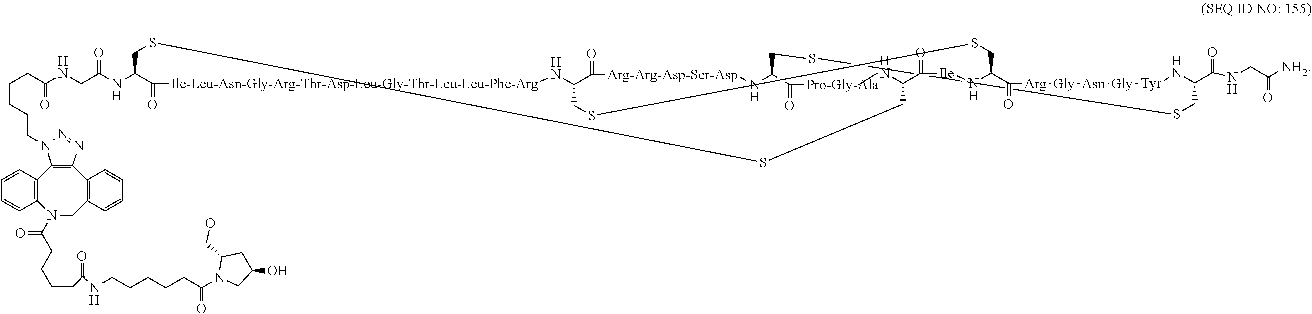

In one embodiment, the ligand comprises a compound of the structure

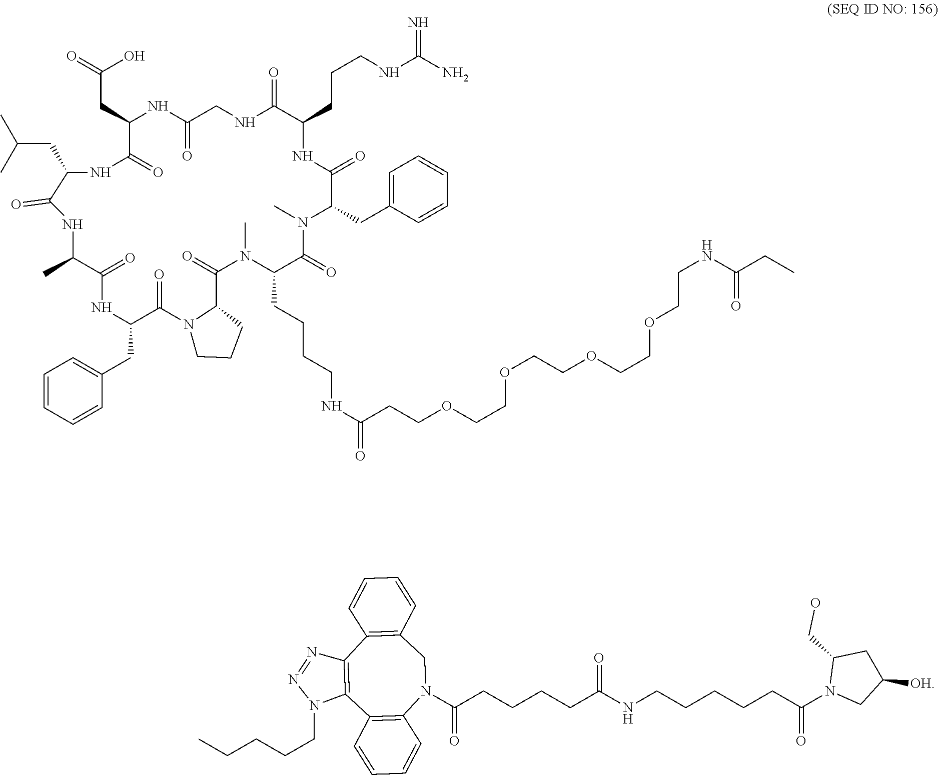

In some embodiments the ligand comprises a compound of Formula (I)

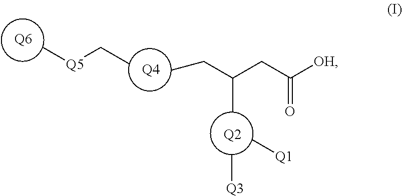

wherein:
Q1 is selected from the group consisting of H, halogen, OR1, NR1R2, an optionally substituted sulfonyl, optionally substituted C1-C6 alkyl, optionally substituted C3-C7 cycloalkyl, optionally substituted heterocyclic, optionally substituted aryl and optionally substituted heteroaryl;
Q2 and Q4 are each independently selected from the group consisting of an optionally substituted C1-C6 alkyl, optionally substituted C3-C7 cycloalkyl, optionally substituted heterocyclic, optionally substituted aryl and optionally substituted heteroaryl;
Q3 is a linker;
Q5 is an optionally substituted C1-C6 alkyl, optionally substituted C3-C7 cycloalkyl, O, or NR1; and
Q6 is optionally substituted C4-C7 cycloalkyl, optionally substituted heterocyclic, optionally substituted aryl or optionally substituted heteroaryl; and
R1 and R2 are each independently selected from the group consisting of H, an optionally substituted carbonyl, optionally substituted alkyl, optionally substituted aryl and optionally substituted heterocyclic group.

In some embodiments the ligand comprises a compound of Formula (II)

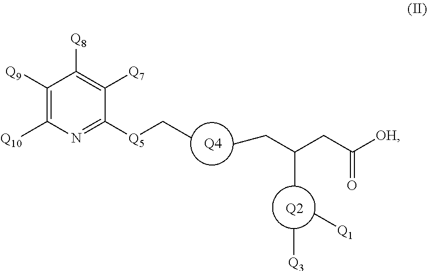

wherein:
Q1, Q7, Q8, Q9, and Q10 are each independently selected from the group consisting of H, halogen, OR1, NR1R2, an optionally substituted sulfonyl, guanidyl, optionally substituted C1-C6 alkyl, optionally substituted C3-C7 cycloalkyl, optionally substituted heterocyclic, optionally substituted aryl and optionally substituted heteroaryl;
Q2 and Q4 are independently selected from the group consisting of an optionally substituted C1-C6 alkyl, optionally substituted C3-C7 cycloalkyl, optionally substituted heterocyclic, optionally substituted aryl and optionally substituted heteroaryl;
Q3 is a linker;
Q5 is optionally substituted C1-C6 alkyl, optionally substituted C3-C7 cycloalkyl, O, or NR1; and
R1 and R2 are each independently selected from the group consisting of H, an optionally substituted carbonyl, optionally substituted alkyl, optionally substituted aryl and optionally substituted heterocyclic group.

Alternatively, Q9 and Q10, together with the atoms to which they are connected, can combine to form an optionally substituted carbocyclic or heterocyclic ring.

In some embodiments the ligand comprises a compound of Formula (III)

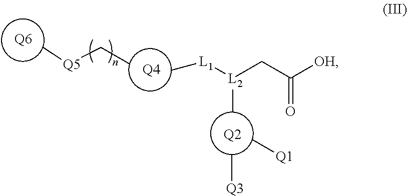

wherein:
Q1 is selected from the group consisting of H, halogen, NR1R2, OR3, an optionally substituted sulfonyl, optionally substituted C1-C6 alkyl, optionally substituted C3-C7 cycloalkyl, optionally substituted heterocyclic, optionally substituted aryl and optionally substituted heteroaryl
Q2 and Q4 are each independently selected from the group consisting of an optionally substituted C1-C6 alkyl, optionally substituted C3-C7 cycloalkyl, optionally substituted heterocyclic, optionally substituted aryl and optionally substituted heteroaryl;
Q3 is a linker;
Q5 is an optionally substituted C1-C6 alkyl, optionally substituted C3-C7 cycloalkyl, O, or NR3;
Q6 is an optionally substituted C4-C7 cycloalkyl, optionally substituted heterocyclic, optionally substituted aryl or optionally substituted heteroaryl.
L1 and L2 are each independently selected from the group consisting of an optionally substituted alkyl, carbonyl, sulfonyl and NR3;
R1 and R2 are each independently selected from the group consisting of H, an optionally substituted carbonyl, an optionally substituted alkyl, an optionally substituted aryl and an optionally substituted heterocyclic group; and
R3 is selected from the group consisting of H, an optionally substituted alkyl, an optionally substituted aryl and an optionally substituted heterocyclic group.

In one embodiment, the ligand comprises the structure:

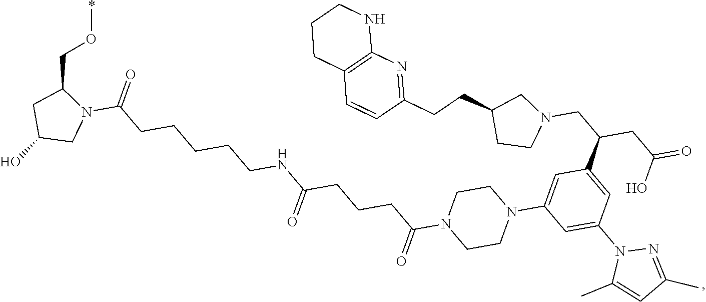 [SMILES: *OC[C@@H]1C[C@@H](O)CN1C(=O)CCCCCNC(=O)CCCC(=O)N1CCN(c2cc([C@H](CC(=O)O)CN3CC[C@@H](CCc4ccc5c(n4)NCCC5)C3)cc(-n3nc(C)cc3C)c2)CC1]

wherein * represents the bond to an oligonucleotide; for example, to the 3′-end of an oligonucleotide via a phosphodiester or phosphorothioate linkage.

In one embodiment, the ligand comprises the structure:

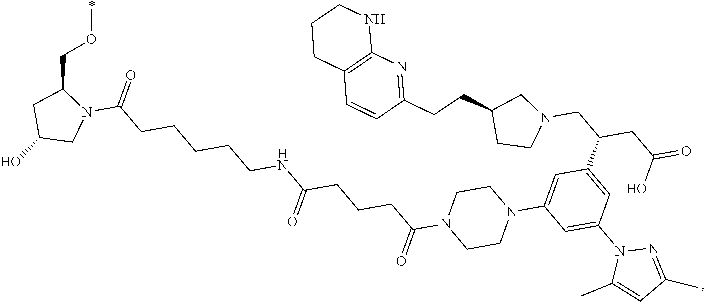 [SMILES: *OC[C@@H]1C[C@@H](O)CN1C(=O)CCCCCNC(=O)CCCC(=O)N1CCN(c2cc([C@@H](CC(=O)O)CN3CC[C@@H](CCc4ccc5c(n4)NCCC5)C3)cc(-n3nc(C)cc3C)c2)CC1]

wherein * represents the bond to an oligonucleotide; for example, to the 3′-end of an oligonucleotide via a phosphodiester or phosphorothioate linkage.

In one embodiment, the ligand comprises the structure:

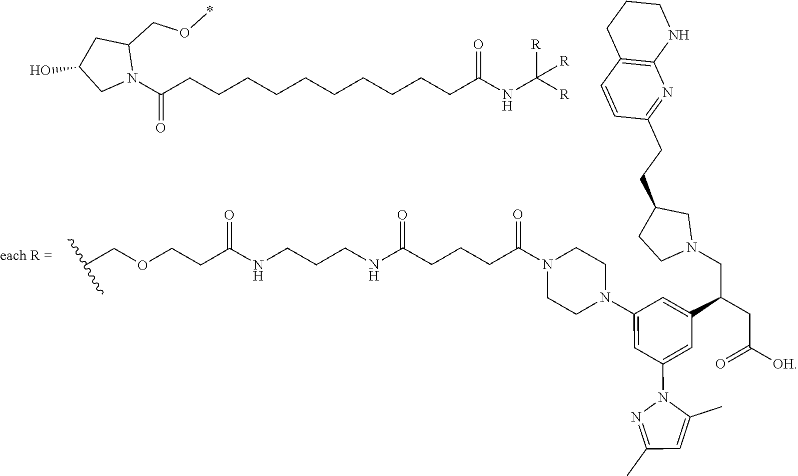

wherein * represents the bond to an oligonucleotide; for example, to the 3′-end of an oligonucleotide via a phosphodiester or phosphorothioate linkage.

In one embodiment, the ligand comprises the structure:

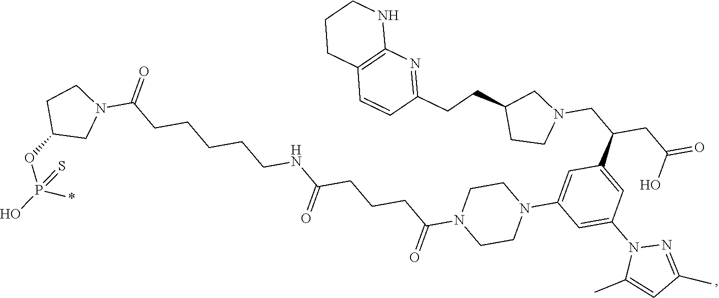 [SMILES: *P(O)(=S)O[C@@H]1CCN(C(=O)CCCCCNC(=O)CCCC(=O)N2CCN(c3cc([C@H](CC(=O)O)CN4CC[C@@H](CCc5ccc6c(n5)NCCC6)C4)cc(-n4nc(C)cc4C)c3)CC2)C1]

or a salt thereof, wherein * represents the bond to an oligonucleotide; for example, to the 5′-end of an oligonucleotide.

In one embodiment, the ligand comprises the structure:

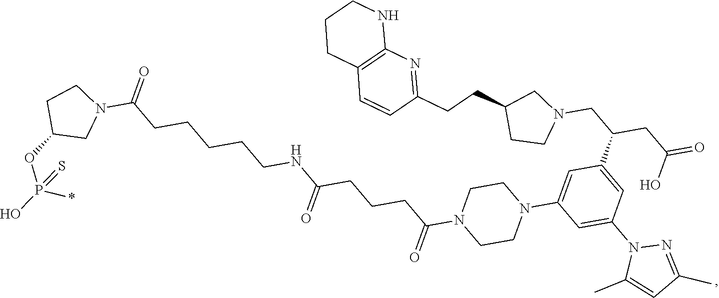 [SMILES: *P(O)(=S)O[C@@H]1CCN(C(=O)CCCCCNC(=O)CCCC(=O)N2CCN(c3cc([C@@H](CC(=O)O)CN4CC[C@@H](CCc5ccc6c(n5)NCCC6)C4)cc(-n4nc(C)cc4C)c3)CC2)C1]

or a salt thereof, wherein * represents the bond to an oligonucleotide; for example, to the 5′-end of an oligonucleotide.

In some embodiments, a suitable ligand and/or ligand and linker for use in the present invention is selected from any one of the compounds shown in the table below:

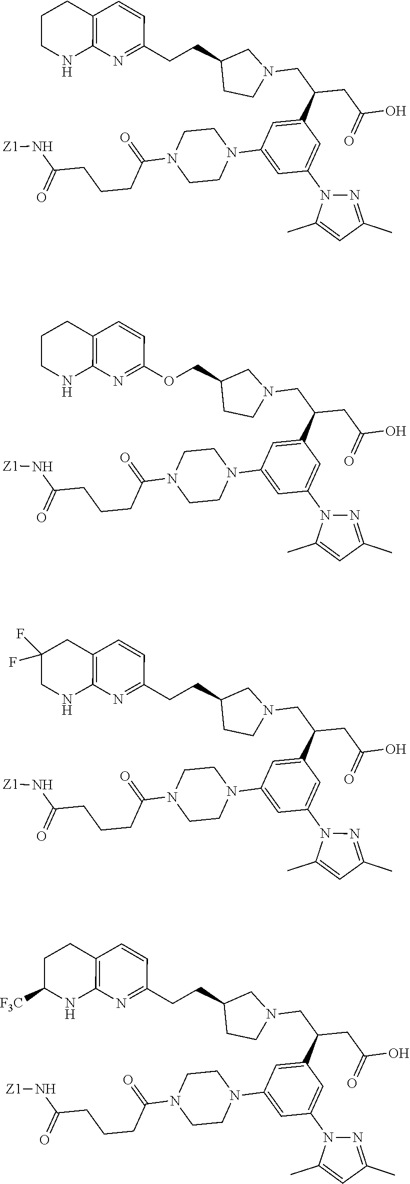

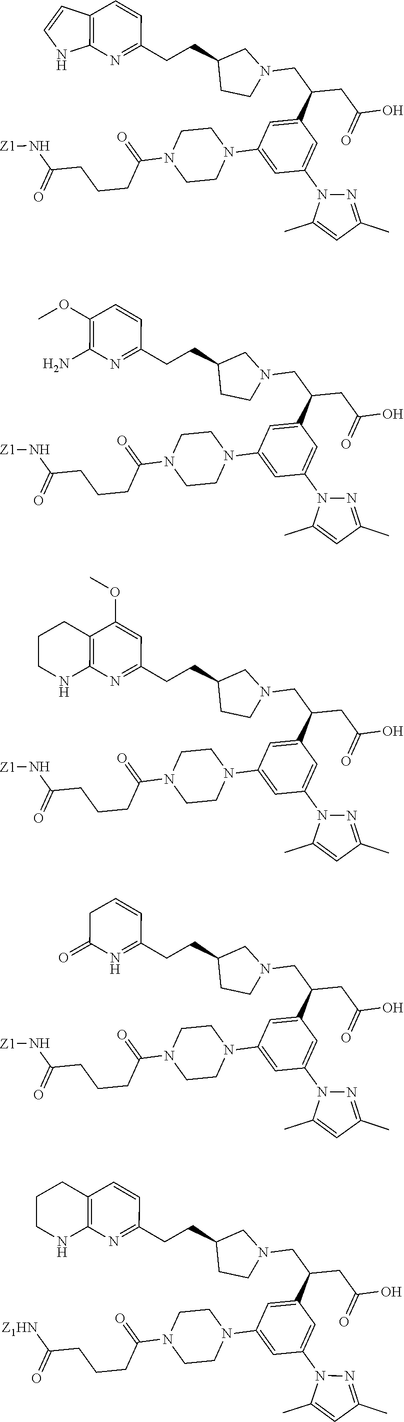

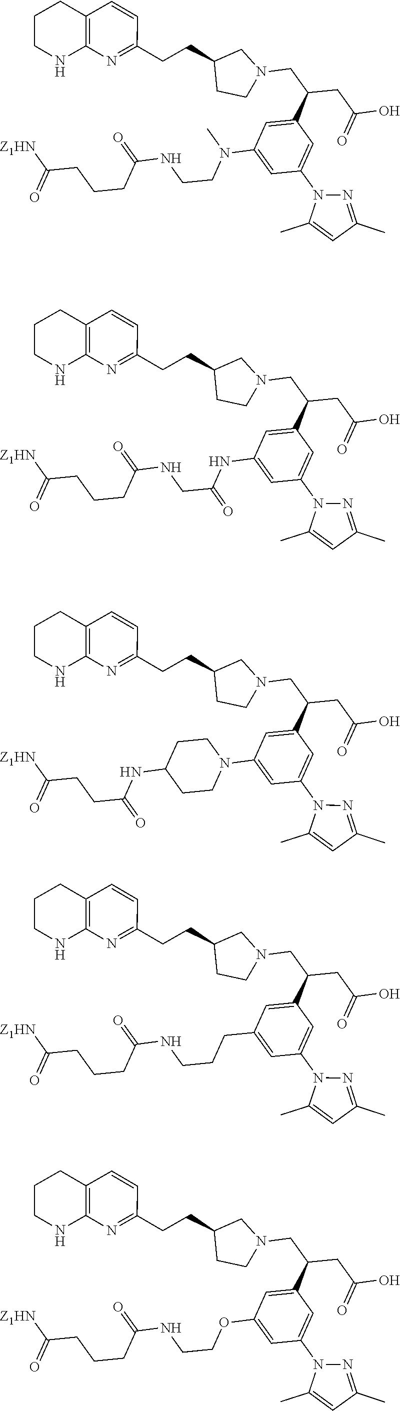

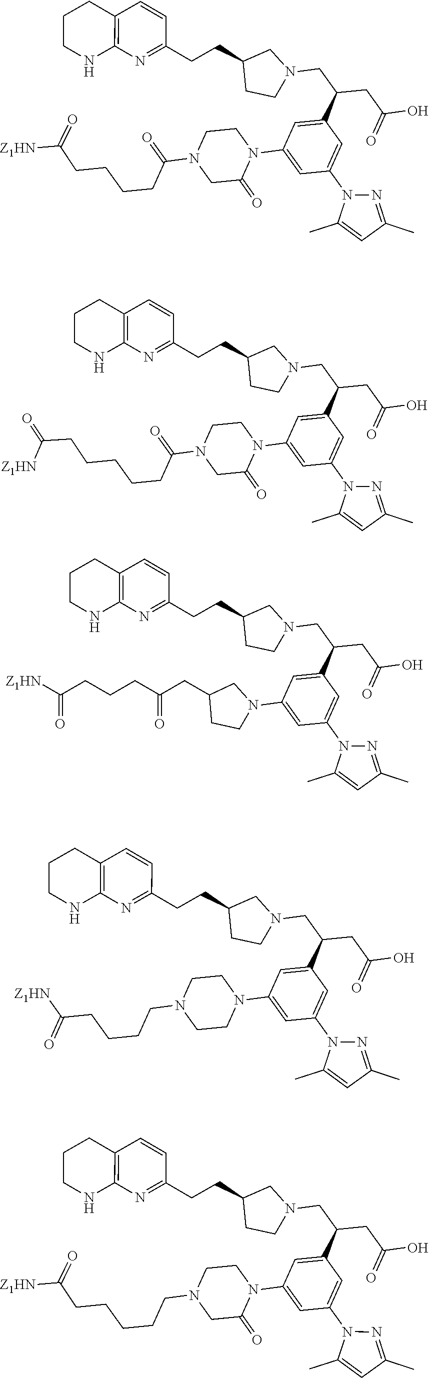

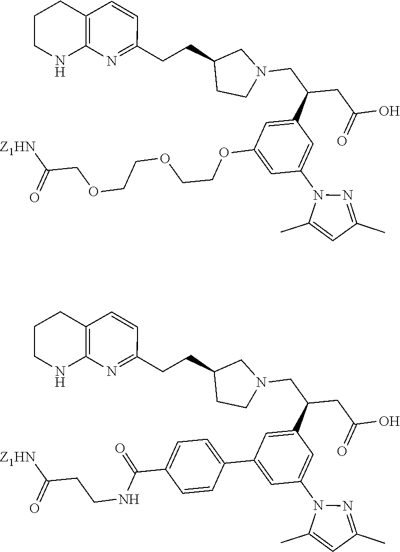

wherein Z1 is a linking group connecting to an oligonucleotide.

In one embodiment, the ligand is conjugated to the sense strand, e.g., the 3′-end of the sense strand; the 5′-end of the sense strand; or both the 5′-end and the 3′-end of the sense strand.

In one embodiment, the ligand is conjugated to an internal position on the sense strand, e.g., a 2′-position on a nucleotide or a modified internucleotide linkage. In one embodiment, the ligand is conjugated to a 2′-position on a nucleotide of the sense strand.

In another embodiment, the ligand is conjugated to the antisense strand, e.g., the 3′-end of the antisense strand; the 5′-end of the antisense strand; or both the 5′-end and the 3′-end of the antisense strand.

In one embodiment, the ligand is conjugated to an internal position on the antisense strand, e.g., a 2′-position on a nucleotide or a modified internucleotide linkage. In one embodiment, the ligand is conjugated to a 2′-position on a nucleotide of the antisense strand.

In one embodiment, the target gene is selected from the group consisting of myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); and Double Homeobox 4 (DUX4), dystrophy myotonic protein kinase (DMPK), glycogen synthase 1 (GYS1), survival of motor neuron 1 (SMN1), alpha-glucosidase (GAA); adrenoceptor beta 1 (ADRB1); calcium voltage-gated channel subunit alpha1 C (CACNA1C); calcium voltage-gated channel subunit alpha1 G (CACNA1G) (T type calcium cchannel); angiotensin II receptor type 1 (AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); or Phosphodiesterase 1 (PDE1).

In one embodiment, the target gene is selected from the group consisting of myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); and Double Homeobox 4 (DUX4), dystrophy myotonic protein kinase (DMPK), glycogen synthase 1 (GYS1), survival of motor neuron 1 (SMN1), and alpha-glucosidase (GAA).

In one embodiment, the target gene is selected from the group consisting of adrenoceptor beta 1 (ADRB1); calcium voltage-gated channel subunit alpha1 C (CACNA1C); calcium voltage-gated channel subunit alpha1 G (CACNA1G) (T type calcium cchannel); angiotensin II receptor type 1 (AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); or Phosphodiesterase 1 (PDE1).

In one embodiment, the target gene is selected from the group consisting of MUC5B, TSLP, IL33, ALOX15, AGER(RAGE), MUC5AC, and STAT6.

In one aspect, the present invention provides cells containing any of the dsRNA agents of the invention.

In another aspect, the present invention provides a pharmaceutical composition for inhibiting expression of the target gene, comprising any of the dsRNA agents of the invention.

In one embodiment, the dsRNA agent is present in a buffer solution, e.g., a buffer solution comprising acetate, citrate, prolamine, carbonate, or phosphate, or any combination thereof. In one embodiment, the buffer solution is phosphate buffered saline (PBS). In other embodiments, the dsRNA agent is present in an unbuffered solution, e.g., in water or normal saline.

In one aspect, the present invention provides a method of inhibiting expression of a target gene in a skeletal muscle cell and/or a cardiac muscle cell. The method includes contacting the cell with any of the dsRNA agents of the invention or any of the pharmaceutical compositions of the invention, e.g., and maintaining the cell produced in step (a) for a time sufficient to obtain degradation of the mRNA transcript of the target gene in the skeletal muscle cell and/or cardiac muscle cell, thereby inhibiting expression of the target gene in the skeletal muscle cell and/or cardiac muscle cell.

In one aspect, the present invention provides a method of inhibiting expression of a target gene in a lung cell. The method includes contacting the cell with any of the dsRNA agents of the invention or any of the pharmaceutical compositions of the invention, e.g., and maintaining the cell produced in step (a) for a time sufficient to obtain degradation of the mRNA transcript of the target gene in the lung cell, thereby inhibiting expression of the target gene in the lung cell.

In one embodiment, the cell is within a subject, e.g., human subject.

In certain embodiments, contacting the cell with the dsRNA agent or pharmaceutical composition inhibits the expression of the target gene by at least about 30%, 40%, 50%, 60%, 70%, 80%, 90%, or 95%.

In one embodiment, inhibiting expression of the target gene decreases the target gene protein level in serum of the subject by at least 30%, 40%, 50%, 60%, 70%, 80%, 90%, or 95%.

In one aspect, the present invention provides a method of treating a subject having a muscle disorder, e.g., a skeletal muscle disorder and/or a cardiac muscle disorder. The method includes administering to the subject a therapeutically effective amount of any of the dsRNA agents of the invention or any of the pharmaceutical compositions of the invention, thereby treating the subject.

In one embodiment, the muscle disorder is selected from the group consisting of Myostatin-related muscle hypertrophy, congenital myasthenic syndrome, facioscapulohumeral muscular dystrophy (FSHD), Spinal Muscular Atrophy (SMA), Myotonic Dystrophy Type 1 (DM1), Pompe disease, PLN cardiomyopathy, spasticity, obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); hypertrophic cardiomyopathy (HCM); and PLN cardiomyopathy.

In one embodiment, the skeletal muscle disorder is selected from the group consisting of Myostatin-related muscle hypertrophy, congenital myasthenic syndrome, facioscapulohumeral muscular dystrophy (FSHD), Spinal Muscular Atrophy (SMA), Myotonic Dystrophy Type 1 (DM1), Pompe disease, PLN cardiomyopathy, and spasticity.

In one embodiment, the cardiac muscle disorder is selected from the group consisting of obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); hypertrophic cardiomyopathy (HCM); and PLN cardiomyopathy.

In one aspect, the present invention provides a method of treating a subject having a lung disorder. The method includes administering to the subject a therapeutically effective amount of any of the dsRNA agents of the invention or any of the pharmaceutical compositions of the invention, thereby treating the subject.

In one embodiment, the lung disorder is selected from the group consisting of pulmonary fibrosis e.g. idiopathic pulmonary fibrosis, non-specific interstitial pneumonia (NSIP), usual interstitial pneumonia (UIP), Hermansky-Pudlak syndrome, progressive massive fibrosis (a complication of coal workers' pneumoconiosis), connective tissue disease-related pulmonary fibrosis, airway fibrosis in asthma and COPD, acute respiratory distress syndrome (ARDS) associated fibrosis, acute lung injury; radiation-induced fibrosis; familial pulmonary fibrosis; pulmonary hypertension, asthma, asthma and chronic rhinosinusitis, and nasal polyps and chronic rhinosinusitis.

The dsRNA agent or pharmaceutical composition may be administered to the subject subcutaneously, intramuscularly, intravenously, or via inhalation.

In one embodiment, the therapeutic methods of the invention further include administering to the subject an additional agent or a therapy suitable for treatment or prevention of an extrahepatic disorder.

### BRIEF DESCRIPTION OF THE DRAWINGS

FIG. 1 are graphs depicting the inhibition of Sod1 mRNA expression in mouse quadriceps, heart, liver, and lung 21 days after a single 2 mg/kg intravenous dose of AD-1427062, AD-1534458, AD-1534460, AD-1481903, AD-1481901, or AD-1481902, or PBS control.

FIG. 2 are graphs depicting the inhibition of Sod1 mRNA expression in mouse gastrocnemius and quadriceps 21 days after a single 2 mg/kg intravenous dose of AD-2032892 or PBS control.

FIG. 3 is a graph depicting the inhibition of Sod1 mRNA expression in cynomolgus monkey heart, quadriceps, liver, gastrocnemius, and kidney 30 days after a single 10 mg/kg intravenous dose of AD-2032892 or PBS control.

FIG. 4 is a graph depicting the inhibition of Sod1 mRNA expression in mouse lung 21 days after a single 0.5 mg/kg intratracheal dose of AD-1481901, AD-2032892, or PBS control.

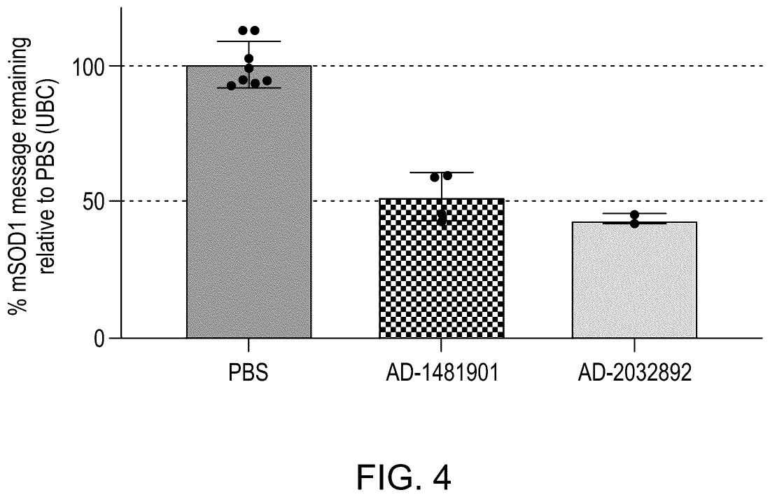

FIG. 5 is a graph depicting the inhibition of Sod1 mRNA expression in mouse lung 21 days after a single 0.5 mg/kg intranasal dose of AD-1481901, AD-2032892, or PBS control.

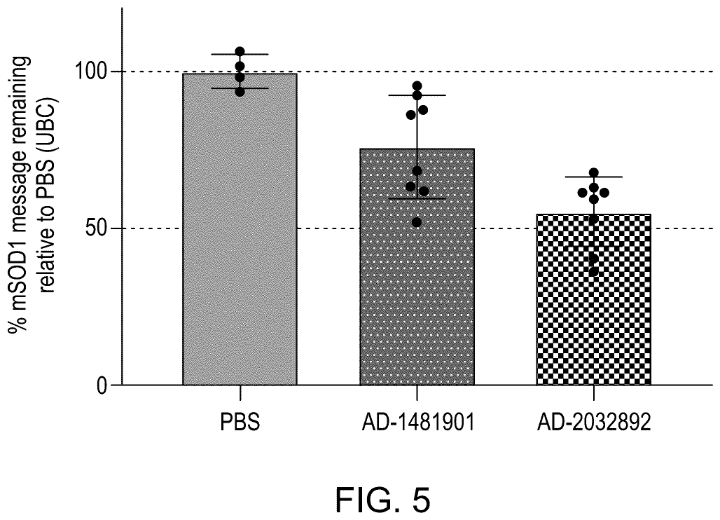

FIG. 6 is a graph depicting the inhibition of Sod1 mRNA expression in mouse lung 21 days after a single 2 mg/kg oropharyngeal aspiration dose of AD-2032892 or PBS control.

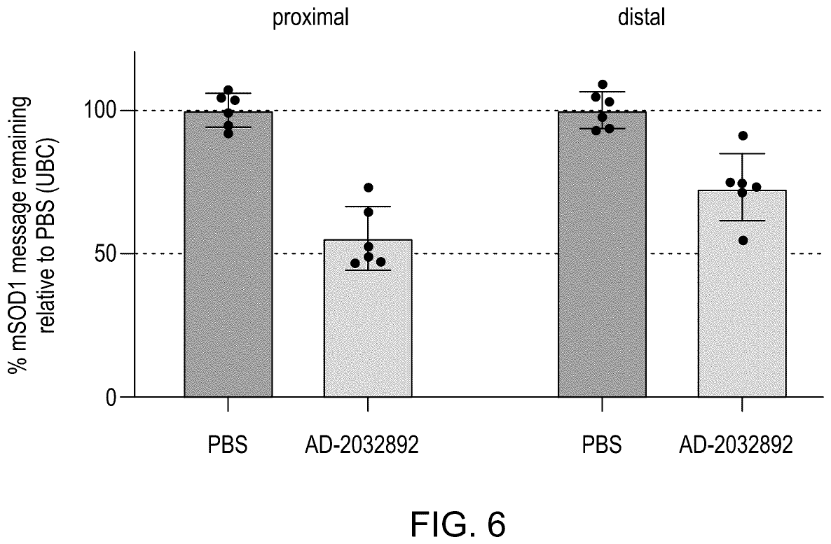

FIG. 7 is a graph depicting the inhibition of Sod1 mRNA expression in cynomolgus monkey lung 30 days after a single 5 mg/kg or 10 mg/kg intravenous dose of AD-2032892 or PBS control.

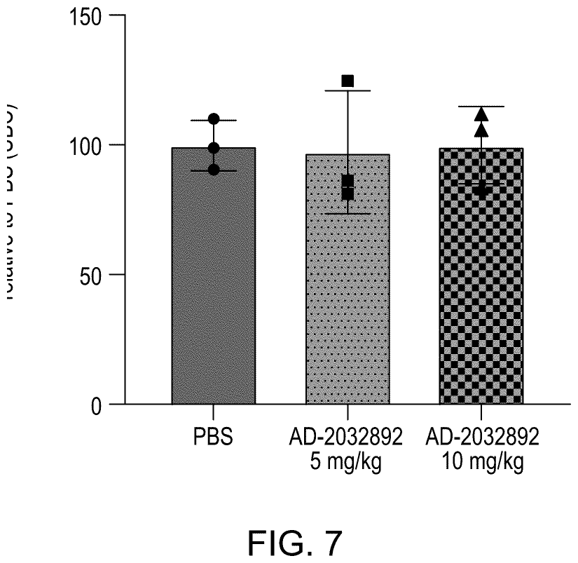

FIG. 8 is a graph depicting the inhibition of Sod1 mRNA expression in mouse lung 10 days after a single 1 mg/kg or 10 mg/kg intranasally administered dose of the indicated agents or PBS control.

FIG. 9 is a comparative illustration of Formulae (IV), (V), (X), (XII), and (XV), herein; embodiments for variables of one Formula are equally applicable to the variable in another formula as delineated by the columns defined by the dotted vertical lines; in each case the variables among the various formulae have the same name, except for L′ of Formula (X) and T of formulas (V) and (XV).

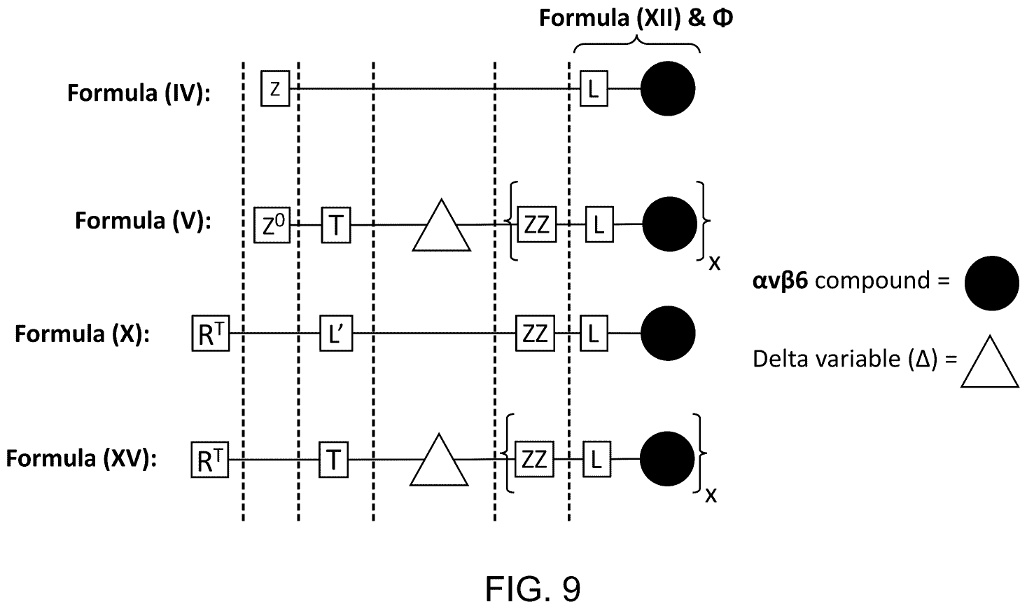

FIG. 10 is an illustration of a process for preparing an oligonucleotide conjugate of the disclosure where a compound of Formula (IV) or (V), each with a first member of a reactive pair (Z, and Z°, respectively) is contacted with an oligonucleotide comprising the second member of the reactive pair (Z′), to provide the conjugated oligonucleotide with a ZZ covalent construct resulting from the reactive pair; the oligonucleotide can be modified with the second member of the reactive pair at the 5′-terminus, the 3′-terminus or at an internal position (e.g., 2′-0 or at an internucleotide linkage of a nucleoside).

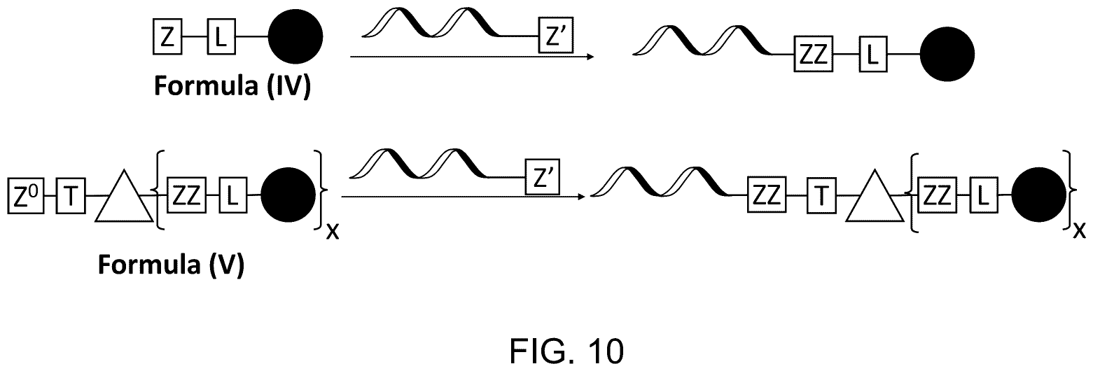

FIG. 11 is an illustration of representative embodiments of Formula (X), wherein R^{T1} is either a phosphorous coupling group (providing, for example, a phosphoramidite); an oligonucleotide connected through a divalent linking group (L^{L}); or a solid-supported ligand, suitable for solid-phase oligonucleotide synthesis where the ligand of Formula (X) is attached to a surface functional group of the solid support via a divalent support linking group (L^{K}); divalent linking group (L^{L}) can connect to the oligonucleotide at the 5′-terminus (e.g., the 5′-0), the 3′-terminus (e.g., the 3′-0), or at an internal position (e.g., 2′-0 or at an internucleotide linkage of a nucleoside).

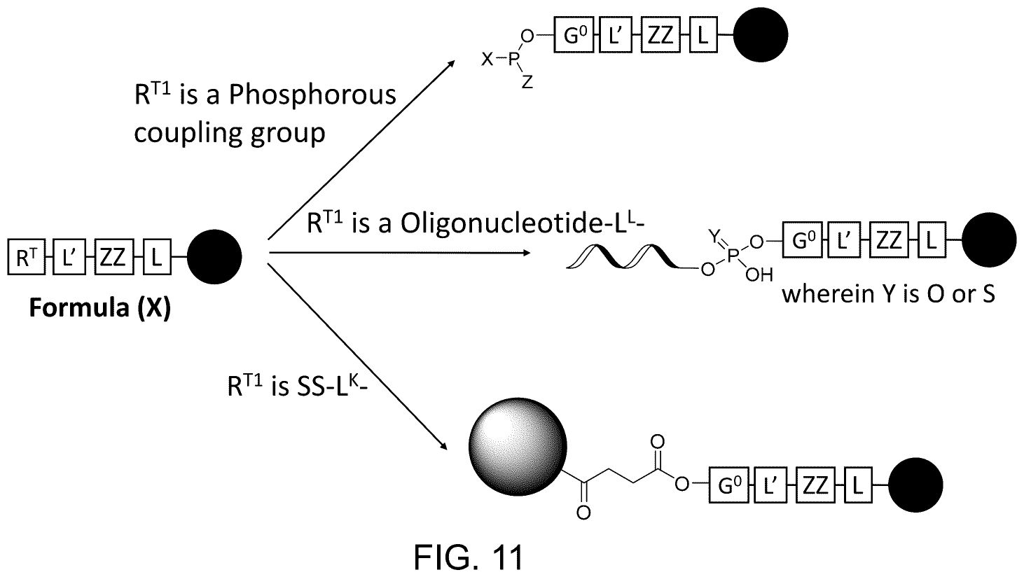

FIG. 12 is an illustration of representative embodiments of Formula (XV), wherein R^{T1} is either a phosphorous coupling group (providing, for example, a phosphoramidite); an oligonucleotide connected through a divalent linking group (L^{L}); or a solid-supported ligand, suitable for solid-phase oligonucleotide synthesis where the ligand of Formula (XV) is attached to a surface functional group of the solid support via a divalent support linking group (L^{K}); divalent linking group (L^{L}) can connect to the oligonucleotide at the 5′-terminus (e.g., the 5′-0), the 3′-terminus (e.g., the 3′-0), or at an internal position (e.g., 2′-0 or at an internucleotide linkage of a nucleoside).

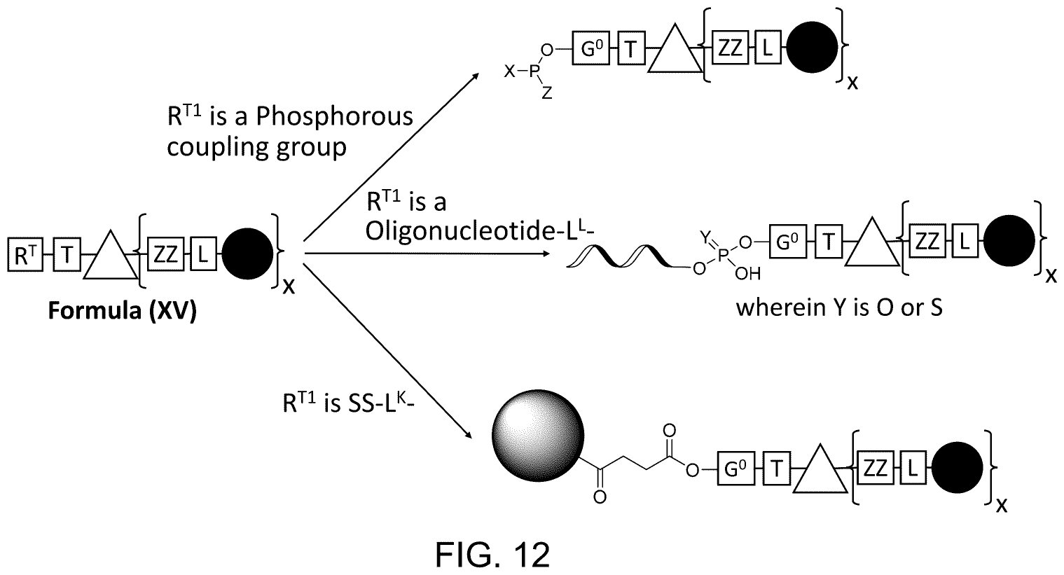

### DETAILED DESCRIPTION

The present invention is based, at least in part, on the discovery of alpha-v-beta-6 (αvβ6) integrin compounds and the surprising discovery that conjugating at least one such alpha-v-beta-6 (αvβ6) integrin compound to at least one strand of a dsRNA agent, e.g., the sense strand, provides surprisingly efficient in vivo delivery to extrahepatic tissue, i.e., muscle tissue, resulting in efficient entry and internalization of the dsRNA agent into extrahepatic tissue, e.g., muscle tissue, e.g., skeletal muscle tissue and/or cardiac muscle tissue, or lung tissue, and surprisingly good inhibition of target gene expression in extrahepatic tissue, e.g., muscle tissue, e.g., skeletal muscle tissue and/or cardiac muscle tissue, or lung tissue.

The following detailed description discloses how to make and use alpha-v-beta-6 (αvβ6) integrin compounds, compositions containing dsRNA agents comprising such compounds that mediates delivery to extrahepatic tissue, e.g., muscle tissue, e.g., skeletal muscle tissue and/or cardiac muscle tissue, or lung tissue, conjugated to at least one strand to inhibit the expression of a target gene as well as compositions, uses, and methods for treating subjects that would benefit from inhibition and/or reduction of the expression of the target gene.

### I. Definitions

In order that the present disclosure may be more readily understood, certain terms are first defined. In addition, it should be noted that whenever a value or range of values of a parameter are recited, it is intended that values and ranges intermediate to the recited values are also intended to be part of this disclosure.

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means +10%. In certain embodiments, about means +5%. When about is present before a series of numbers or a range, it is understood that “about” can modify each of the numbers in the series or range.

The term “at least”, “no less than”, or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a sequence and its indicated site on a transcript or other sequence, the nucleotide sequence recited in the specification takes precedence.

Terms used herein may be preceded and/or followed by a single dash, or a double dash, “=”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” unless a dash indicates otherwise. For example, C₁₋₆alkoxycarbonyloxy and —OC(O)C₁₋₆alkyl indicate the same functionality; similarly arylalkyl, arylalkyl-, and -alkylaryl indicate the same functionality.

Further, certain terms herein may be used as both monovalent and divalent linking radicals as would be familiar to those skilled in the art, and by their presentation linking between two other moieties. For example, an alkyl group can be both a monovalent radical or divalent radical; in the latter case, it would be apparent to one skilled in the art that an additional hydrogen atom is removed from a monovalent alkyl radical to provide a suitable divalent moiety.

Throughout the disclosure, [symbol] is used to represent an oligonucleotide; such oligonucleotides may be an RNA, a DNA, a single-stranded RNA, such as an antisense oligonucleotide (ASO), a double-stranded RNA, such as an siRNA, and oligonucleotide derivatives such as phosphorodiamidate morpholino oligomers (PMOs).

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkynyl” as used herein means a straight or branched hydrocarbon chain containing from 2 to 10 carbons, unless otherwise specified, and containing at least one carbon-carbon triple bond. Representative examples of alkynyl include, but are not limited to, 1-butynyl, 2-butynyl, 1-propynyl and the like.

The term “alkoxy” as used herein, means an alkyl group, as defined herein, appended to the parent molecular moiety through an oxygen atom. Representative examples of alkoxy include, but are not limited to, methoxy, ethoxy, n-propoxy, 2-propoxy, n-butoxy, tert-butoxy, n-pentyloxy, and n-hexyloxy.

The term “alkyl” as used herein, means a straight or branched chain hydrocarbon containing from 1 to 10 carbon atoms, unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a divalent linking group between two other moieties, then it may also be a straight or branched chain; examples include, but are not limited to —CH₂—, —CH₂CH₂—, —CH₂CH₂CHC(CH₃)—, —CH₂CH(CH₂CH₃)CH₂—.

The term “aryl,” as used herein, means a phenyl (i.e., monocyclic aryl); naphthyl or azulenyl; a bicyclic ring system containing a phenyl fused to a cycloalkyl, cycloalkenyl, or heterocyclyl ring. Representative examples of the bicyclic aryls include, but are not limited to, azulenyl, naphthyl, 2,3-dihydroinden-1-yl, 2,3-dihydroinden-2-yl, 2,3-dihydroinden-3-yl, 2,3-dihydroinden-4-yl, 2,3-dihydroinden-5-yl, 2,3-dihydroindol-1-yl, indolin-2-yl, indolin-3-yl, indolin-4-yl, indolin-5-yl, indolin-6-yl, indolin-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-2-yl, 2,3-dihydrobenzofuran-3-yl, 2,3-dihydrobenzofuran-4-yl, 2,3-dihydrobenzofuran-5-yl, 2,3-dihydrobenzofuran-6-yl, 2,3-dihydrobenzofuran-7-yl, 2,3-dihydrobenzothien-2-yl, 2,3-dihydrobenzothien-3-yl, 2,3-dihydrobenzothien-4-yl, 2,3-dihydrobenzothien-5-yl, 2,3-dihydrobenzothien-6-yl, 2,3-dihydrobenzothien-7-yl, 2,3-dihydrobenzothien-8-yl, benzo[d][1,3]dioxol-4-yl, benzo[d][1,3]dioxol-5-yl, 2H-chromen-2-on-3-yl, 2H-chromen-2-on-4-yl, 2H-chromen-2-on-5-yl, 2H-chromen-2-on-6-yl, 2H-chromen-2-on-7-yl, 2H-chromen-2-on-8-yl, isoindoline-1,3-dion-2-yl, isoindoline-1,3-dion-4-yl, isoindoline-1,3-dion-5-yl, inden-1-on-2-yl, inden-1-on-3-yl, inden-1-on-4-yl, inden-1-on-5-yl, inden-1-on-6-yl, inden-1-on-7-yl, 2,3-dihydrobenzo[b][1,4]dioxan-2-yl, 2,3-dihydrobenzo[b][1,4]dioxan-5-yl, 2,3-dihydrobenzo[b][1,4]dioxan-6-yl, 2H-benzo[b][1,4]oxazin-3(4H)-on-5-yl, 2H-benzo[b][1,4]oxazin-3(4H)-on-6-yl, 2H-benzo[b][1,4]oxazin-3(4H)-on-7-yl, 2H-benzo[b][1,4]oxazin3(4H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-3-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl.

In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The term “arylalkyl,” “aralkyl”, “-alkylaryl,” and “arylalkyl-” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “azido” means a —N₃ group.

The term “carboxy” means a —COOH group.

The terms “cyano” and “nitrile” as used herein, mean a —CN group.

The term “cycloalkyl” as used herein, means a monocyclic or a bicyclic cycloalkyl ring system.

Monocyclic ring systems are cyclic hydrocarbon groups containing from 3 to 10 carbon atoms, where such groups are saturated. Examples of monocyclic cycloalkyls include cyclopropyl, cyclobutyl, cyclopentyl, cyclohexyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bridged bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to a monocyclic cycloalkyl. Representative examples of fused bicyclic ring systems include, but are not limited to, decaliyl. Cycloalkyl groups are optionally substituted with one or two groups which are independently oxo or thia. In certain embodiments, the fused bicyclic cycloalkyl is a 5 or 6 membered monocyclic cycloalkyl ring fused to a 5 or 6 membered monocyclic cycloalkyl, wherein the fused bicyclic cycloalkyl is optionally substituted by one or two groups which are independently oxo or thia.

“Cycloalkenyl” as used herein refers to a monocyclic or a bicyclic cycloalkenyl ring system. Monocyclic ring systems are cyclic hydrocarbon groups containing from 3 to 8 carbon atoms, where such groups are unsaturated (i.e., containing at least one annular carbon-carbon double bond), but not aromatic. Examples of monocyclic ring systems include cyclopentenyl and cyclohexenyl. Bicyclic cycloalkenyl rings are bridged monocyclic rings or a fused bicyclic rings. Bridged monocyclic rings contain a monocyclic cycloalkenyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic cycloalkenyls include, but are not limited to, norbornenyl and bicyclo[2.2.2]oct-2-enyl. Fused bicyclic cycloalkenyl ring systems contain a monocyclic cycloalkenyl ring fused to either a monocyclic cycloalkyl or a monocyclic cycloalkenyl. Cycloalkenyl groups are optionally substituted with one or two groups which are independently oxo or thia.

The term “monocyclic ring”, as used herein, comprises monocyclic aryl, monocyclic cycloalkyl, monocyclic cycloalkenyl and monocyclic heterocyclyl.

The term “halo” or “halogen” as used herein, means —Cl, —Br, —I or —F.

The term “H” means hydrogen.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic ring system containing at least one heteroaromatic ring (i.e., a monocyclic or bicyclic aromatic ring system containing at least one heteroatom within the aromatic system). The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroquinolin-8-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroquinolin-5-yl, 5,6,7,8-tetrahydroquinolin-6-yl, 5,6,7,8-tetrahydroquinolin-7-yl, 5,6,7,8-tetrahydroquinolin-8-yl, 5,6,7,8-tetrahydroisoquinolin-1-yl, thienopyridinyl, 4,5,6,7-tetrahydrobenzo[c][1,2,5]oxadiazol-4-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The term “heteroarylalkyl” and “-alkylheteroaryl” as used herein, means a heteroaryl, as defined herein, appended to the parent molecular moiety through an alkyl group, as defined herein.

Representative examples of heteroarylalkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, pyridin-4-ylmethyl, pyrimidin-5-ylmethyl, 2-(pyrimidin-2-yl)propyl, thien-2-ylmethyl, and thien-3-ylmethyl.

The term “heterocyclyl” as used herein, means a monocyclic heterocycle or a bicyclic heterocycle. The monocyclic heterocycle is a 3, 4, 5, 6, or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one, or two double bonds and one, two or three heteroatoms selected from the group consisting of O, N and S. Representative examples of monocyclic heterocycles include, but are not limited to, azetidinyl, azepanyl, aziridinyl, diazepanyl, 1,3-dioxan-2-yl, 1,3-dioxolan-2-yl, 1,3-dithiolan-2-yl, 1,2-dithiolan-3-yl, 1,2-dithiolan-4-yl, 1,3-dithian-2-yl, 1,2-dithian-3-yl, 1,2-dithian-4-yl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a monocyclic cycloalkyl, a monocyclic cycloalkenyl, or a monocyclic heterocycle. Representative examples of bicyclic heterocyclyls include, but are not limited to, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to a 5 or 6 membered monocyclic cycloalkyl, a 5 or 6 membered monocyclic cycloalkenyl, or a 5 or 6 membered monocyclic heterocyclyl, wherein the bicyclic heterocyclyl is optionally substituted by one or two groups which are independently oxo or thia.

The term “hydroxy” or “hydroxyl” as used herein means an —OH group. The term “thiol” as used herein means an —SH group.

The term “nitro” as used herein means a —NO₂ group.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “thia” as used herein means a ═S group.

The term “amine” or “amino” encompasses compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “unsaturated” as used herein means the referenced chemical structure contains at least one multiple carbon-carbon bond (i.e., double or triple bond, or both), but is not aromatic. For example, an unsaturated cycloalkyl group as defined herein includes cyclohexenyl, cyclopentenyl, cyclohexadienyl, and the like.

The term “leaving group” as used herein means an atom or group (charged or uncharged) that becomes detached from an atom in what is considered to be the residual or main part of the substrate in a specified reaction. For example, the specified reaction herein, unless otherwise noted, is an S_{N}1 or an S_{N}2 reaction as is understood by one skilled in the art. In certain embodiments, the specified reaction herein, is an S_{N}2 reaction.

The term“substituted” as used herein, whether preceded by the term“optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a“substituted” group has a substituent at one or more substitutable positions of the group, and when more than one position in any given structure is substituted, the substituent is either the same or different at each position. Suitable substituents include, but are not limited to, halogen, hydroxy, thiol, nitro, alkoxy, azido, carboxy, cyano, amino, C₁₋₆alkyl, C₂₋₆alkenyl, C₁₋₆alkoxy, C₁₋₆alkylamino. The term “support linking group as used herein means a divalent chemical moiety that covalent connects a surface-bound functional group of a solid support (e.g., an amino group of an amino-modified solid support) to another chemical moiety.

The term “reactive pair” as used herein means two functional groups known by one skilled in the art to be capable of reacting, alone or in the presence of other reagents, to form a covalent linkage between the two chemical entities which each contain one of member of the reactive pair, respectively; the latter, herein, is referred to as “linking group formed by a reactive pair”. In some embodiments, a reactive pair is a click pair, i.e., two functional groups capable of reacting in a click reaction to form a covalent linkage.

The term “Michael acceptor” as used herein means an alpha, beta-unsaturated compound capable of reacting with a nucleophile at the beta-carbon of the electrophilic alkene of the alpha, beta-unsaturated compound. Examples of alpha, beta-unsaturated compound include, but are not limited to, such as an alpha, beta-unsaturated aldehyde, ester, amide, sulfonyl, ketone, nitrile, or nitro. “Alpha, beta-unsaturated” refers to the carbon-carbon multiple bond that connects the carbon atom that is immediately adjacent to the referenced aldehyde, ester, amide, sulfonyl, ketone, nitrile, or nitro group, to its adjacent carbon atom. Examples of Michael acceptor group include, but are not limited to, N-maleimide, acrylaldehyde, acrylonitrile, acrylic acid, acrylamide (e.g., N-isoproprylacrylamide), acrylate esters (e.g., methyl acrylate), vinyl sulfones, vinylsulfonates, and vinylsulfonamides.

The term “hydroxyl protecting group” or “hydroxyl protecting group” as used herein means those functional groups that are well known in the art and include those described in detail, for example, in Protecting Groups in Organic Synthesis, T. W. Greene and P. G. M. Wuts, 3rd edition, John Wiley & Sons, 1999, the entirety of which is incorporated herein by reference. Suitable hydroxyl protecting groups include but are not limited to, acetyl, trifluoroacetyl, trichloroacetyl, pivaloyl, t-butyl, allyl, optionally substituted benzyl (such as benzyl, 2-nitrobenzyl, 4-nitrobenzyl, 2,6-dichlorobenzyl, 4-chlorobenzyl, 4-fluorobenzyl, 4-bromobenzyl, 4-methoxybenzyl, 3,4-dimethoxybenzyl, 2-cyanobenzyl, 4-cyanobenzyl, 4-phenylbenzyl), 2-picolyl, 4-picolyl, methoxymethyl (MOM), methylthiomethyl (MTM), ethoxymethyl, 2-methoxyethoxymethyl (MEM), 2,2,2-trichloroethoxymethyl, t-butoxymethyl, benzyloxymethyl (BOM), 4-methoxybenzyloxymethyl (Mbom), (phenyldimethylsilyl)methoxymethyl (SMOM), 2-(Trimethylsilyl)ethoxymethyl (SEM), t-butylthiomethyl, 2-tetrahydropyranyl (THP), 4-methoxytetrahydropyran-2-yl (MTHP), 4-methoxytetrahydrothiopyran-2-yl, 3-bromotetrahydropyran-2-yl, 2-tetrahydrothiopyranyl, trimethylsilyl (TMS), triethylsilyl (TES), triisopropylsilyl (TIPS), t-butyldimethylsilyl (TBDMS), t-butyldiphenylsilyl (TBDPS), Isopropyldimethylsilyl (IPDMS), diethylisopropylsilyl (DEIPS), diphenylmethyl, 9-phenylxanthine-9-yl (Pixyl), 9-(p-methoxyphenyl)xanthine-9-yl (MOX), and optionally substituted trityl (e.g., trityl (Trt), 2-chlorotrityl (Clt), 4-methoxytrityl (Mint), 4-methyltrityl (Mtt), 4,4′-dimethoxytrityl (DMT)), and 4,4′,4″-trimethoxytrityl.

The term “nitrogen protecting group” as used herein means those functional groups that are well known in the art and include those described in detail, for example, in Protecting Groups in Organic Synthesis, T. W. Greene and P. G. M. Wuts, 3rd edition, John Wiley & Sons, 1999, the entirety of which is incorporated herein by reference. Suitable nitrogen protecting groups include but are not limited to, allyl (Alloc), acetyl (Ac), Adpoc (1-(1-Adamantyl)-1-methylethoxycarbonyl), Boc (tert-butyloxy carbonyl), Dde

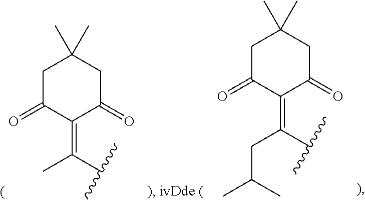 [SMILES: *C(C)=C1C(=O)CC(C)(C)CC1=O ; *C(CC(C)C)=C1C(=O)CC(C)(C)CC1=O]

Dnp (2,4-dinitrophenyl), Mint (4-methoxytrityl), Mtt (4-methyltrityl), Teoc (2-trimethylsilylethoxycarbonyl, Tfa (trifluoroacetyl), optionally substituted trityl (e.g., trityl (Trt), 2-chlorotrityl (Clt), 4-methoxytrityl (Mmt), 4-methyltrityl (Mtt), 4,4′-dimethoxytrityl (DMT)), and 4,4′,4″-trimethoxytrityl), optionally substituted benzyloxycarbonyl (e.g., benzyloxycarbonyl (Z) or 2-chlorobenzyloxycarbonyl (2ClZ)), fluorenylmethoxycarbonyl (Fmoc), methoxyacetyl (mac), phenoxyacetyl (pac), 2-chlorophenoxyacetyl, 3-chlorophenoxyacetyl, 4-chlorophenoxyacetyl, 2,4-dichlorophenoxyacetyl, 2-methylphenoxyacetyl, 3-methylphenoxyacetyl, 4-methylphenoxyacetyl, 4-chloro-2-methylphenoxyacetyl, 2-nitrophenoxyacetyl, 3-nitrophenoxyacetyl, 4-nitrophenoxyacetyl, 2-isopropylphenoxyacetyl, 3-isopropylphenoxyacetyl, 4-isopropylphenoxyacetyl, 2-(t-butyl)phenoxyacetyl, 3-(t-butyl)phenoxyacetyl, 4-(t-butyl)phenoxyacetyl, 2-fluorophenoxyacetyl, 3-fluorophenoxyacetyl, 4-fluorophenoxyacetyl, 2,4-difluorophenoxyacetyl, 4-(trifluoromethoxy)phenoxyacetyl, 2-phenoxypropanoyl, 2-(4-chloro-2-methylphenoxy)propanoyl, and 2-(4-chlorophenoxy)propanoyl.

The term “phosphorous coupling group” as used herein means a H-phosphonate or phosphoroamidite that is reactive with hydroxyl groups and can form a phosphite triester or thiophosphate triester when used within a process for making internucleotide linkages, such as phosphodiester or phosphorothioate linkages.

The term “solid support as used herein refers to any form of a polymer or composite material that does not completely dissolve in a solvent. By way of example only, a solid support includes colloids (isolated or in suspension), gels, resins, films, as well as any other form of a polymer or composite materials that retains a distinct identity apart from the solvent. The term is not meant to limit in any manner the size, shape, form, or chemical structure of the polymeric or composite material. Such polymeric or composite materials are well known in the art, including, by way of example only, cellulose, pore-glass, silica, polystyrene, polystyrene cross-linked with divinylbenzene, polyacrylamide, latex, dimethylacrylamide, dimethylacrylamide cross-linked with N,N′-bis-acryloyl ethylene diamine, glass, glass coated with a hydrophobic polymer, composites, or any other material conventionally used in solid phase organic synthesis. In addition, the term solid support is not limited by the presence and nature of cross-linking groups, and by the nature of the exposed functional groups. Exposed functional groups are moieties on the solid support that can react with a guest molecule to form support-bound guest molecules; preferred exposed functional groups include —OH, —SH, —NH₂, silyloxy, alkylamino, NH₂NH, COOH, ester, aldehyde, —Br, —I, halomethyl (e.g., bromomethyl), and alkenyl. Furthermore, the exposed functional groups can be located on the surface of the solid support or dispersed throughout the solid support. In one embodiment, the solid support has a rigid or semi-rigid surface. Particular examples of solid supports include amino-terminated Controlled Pore Glass (CPG), such as long chain alkylamine CPG (LCAA-CPG), and amino-terminated or hydroxy-terminate cross-linked polystyrene, such as NittoPhase™ solid support (about 420 mol/g hydroxy groups), NittoPhase® HL solid support (about 550 mol/g hydroxy groups), and NittoPhase® UnyLinker™ solid support, each available from Kinovate Life Sciences (Oceanside, CA).

The term “activated ester” as used herein refers to those derivatives of a carboxyl group that are more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy, sulfosuccinimidyloxy, -1-oxybenzotriazolyl; 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy). In certain embodiments, activated esters include succinimidyloxy and sulfosuccinimidyloxy esters.

As used herein, “target sequence” or “target nucleic acid” refers to a contiguous portion of the nucleotide sequence of an mRNA molecule formed during the transcription of a target gene, including mRNA that is a product of RNA processing of a primary transcription product. In one embodiment, the target portion of the sequence will be at least long enough to serve as a substrate for RNAi-directed cleavage at or near that portion of the nucleotide sequence of an mRNA molecule formed during the transcription of a target gene. In one embodiment, the target sequence is within the protein coding region of the target gene. In another embodiment, the target sequence is within the 3′ UTR of the target gene. The target nucleic acid can be a cellular gene (or mRNA transcribed from the gene) whose expression is associated with a particular disorder or disease state.

The target sequence may be from about 9-36 nucleotides in length, e.g., about 15-30 nucleotides in length. For example, the target sequence can be about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In some embodiments, the target sequence is about 19 to about 30 nucleotides in length. In other embodiments, the target sequence is about 19 to about 25 nucleotides in length. In still other embodiments, the target sequence is about 19 to about 23 nucleotides in length. In some embodiments, the target sequence is about 21 to about 23 nucleotides in length. Ranges and lengths intermediate to the above recited ranges and lengths are also contemplated to be part of the invention.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, and uracil can be replaced by other moieties without substantially altering the base pairing properties of an oligonucleotide comprising a nucleotide bearing such replacement moiety. It is understood that when a cDNA sequence is provided, the corresponding mRNA or RNAi agent would include a U in place of a T. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention. Further, one of skill in the art that a T is a target gene sequence, or reverse complement thereof, would often be replaced by a U in an RNAi agent of the invention.

The terms “iRNA”, “RNAi agent,” “iRNA agent,” “RNA interference agent” as used interchangeably herein, refer to an agent that contains RNA as that term is defined herein, and which mediates the targeted cleavage of an RNA transcript via an RNA-induced silencing complex (RISC) pathway. RNA interference (RNAi) is a process that directs the sequence-specific degradation of mRNA. RNAi modulates, e.g., inhibits, the expression of a target gene in a cell, e.g., a cell within a subject, such as a mammalian subject.

In one embodiment, an RNAi agent of the disclosure includes a single stranded RNAi that interacts with a target RNA sequence, e.g., a target mRNA sequence, to direct the cleavage of the target RNA. Without wishing to be bound by theory it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15:485). Dicer, a ribonuclease-III-like enzyme, processes these dsRNA into 19-23 base pair short interfering RNAs with characteristic two base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the disclosure relates to a single stranded RNA (ssRNA) (the antisense strand of a siRNA duplex) generated within a cell and which promotes the formation of a RISC complex to effect silencing of the target gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, an “RNAi agent” for use in the compositions and methods of the disclosure is a double stranded RNA and is referred to herein as a “double stranded RNAi agent,” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., a target mRNA sequence. In some embodiments of the disclosure, a double stranded RNA (dsRNA) triggers the degradation of a target RNA, e.g., an mRNA, through a post-transcriptional gene-silencing mechanism referred to herein as RNA interference or RNAi.

In general, a dsRNA molecule can include ribonucleotides, but as described in detail herein, each or both strands can also include one or more non-ribonucleotides, e.g., a deoxyribonucleotide, a modified nucleotide. In addition, as used in this specification, an “RNAi agent” may include ribonucleotides with chemical modifications; an RNAi agent may include substantial modifications at multiple nucleotides.

As used herein, the term “modified nucleotide” refers to a nucleotide having, independently, a modified sugar moiety, a modified internucleotide linkage, or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a siRNA type molecule, are encompassed by “RNAi agent” for the purposes of this specification and claims.

In certain embodiments of the instant disclosure, inclusion of a deoxy-nucleotide—which is acknowledged as a naturally occurring form of nucleotide—if present within a RNAi agent can be considered to constitute a modified nucleotide.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at at least 4, at least 5, at least 6, at least 7, at least 8, at least 9, at least 10, at least 20, at least 23 or more unpaired nucleotides or nucleotides not directed to the target site of the dsRNA. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

In certain embodiment, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)n; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)4, (U)4, and (dT)4, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one embodiment, an RNAi agent of the invention is a dsRNA, each strand of which is 24-30 nucleotides in length, that interacts with a target RNA sequence, e.g., a target mRNA sequence, to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188).

In one embodiment, an RNAi agent of the invention is a dsRNA agent, each strand of which comprises 19-23 nucleotides that interacts with a target mRNA sequence to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). In one embodiment, an RNAi agent of the invention is a dsRNA of 24-30 nucleotides that interacts with a target mRNA sequence to direct the cleavage of the target RNA.

As used herein, the term “nucleotide overhang” refers to at least one unpaired nucleotide that protrudes from the duplex structure of a RNAi agent, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one embodiment of the dsRNA, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In one embodiment, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′ end of the sense strand of the duplex. In certain embodiments, an extended overhang is present on the 5′ end of the sense strand of the duplex. In certain embodiments, an extended overhang is on the antisense strand of the duplex. In certain embodiments, an extended overhang is present on the 3′ end of the antisense strand of the duplex. In certain embodiments, an extended overhang is present on the 5′ end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

The terms “blunt” or “blunt ended”, as used herein in reference to a dsRNA, mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double stranded over its entire length.

The term “antisense strand” or “guide strand” refers to the strand of an iRNA, e.g., a dsRNA, which includes a region that is substantially complementary to a target sequence, e.g., a target mRNA sequence.

As used herein, the term “region of complementarity” refers to the region on the antisense strand that is substantially complementary to a sequence, for example a target sequence, e.g., a target nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- or 3′-terminus of the RNAi agent.

In some embodiments, a double stranded RNA agent of the invention includes a nucleotide mismatch in the antisense strand. In some embodiments, the antisense strand of the double stranded RNA agent of the invention includes no more than 4 mismatches with the target mRNA, e.g., the antisense strand includes 4, 3, 2, 1, or 0 mismatches with the target mRNA. In some embodiments, the antisense strand double stranded RNA agent of the invention includes no more than 4 mismatches with the sense strand, e.g., the antisense strand includes 4, 3, 2, 1, or 0 mismatches with the sense strand. In some embodiments, a double stranded RNA agent of the invention includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent as described herein can contain one or more mismatches to the target sequence. In one embodiment, a RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a target gene, generally does not contain any mismatch within the central 13 nucleotides. The methods described herein or methods known in the art can be used to determine whether an RNAi agent containing a mismatch to a target sequence is effective in inhibiting the expression of a target gene. Consideration of the efficacy of RNAi agents with mismatches in inhibiting expression of a target gene is important, especially if the particular region of complementarity in a target gene is known to vary.

The term “sense strand” or “passenger strand” as used herein, refers to the strand of a RNAi agent that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can be, for example, “stringent conditions”, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNAi agent, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression, in vitro or in vivo. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

The terms “complementary,” “fully complementary” and “substantially complementary” herein can be used with respect to the base matching between the sense strand and the antisense strand of a dsRNA, or between two oligonucleotides or polynucleotides, such as the antisense strand of a RNAi agent and a target sequence, as will be understood from the context of their use.

As used herein, a polynucleotide that is “substantially complementary to at least part of” a messenger RNA (mRNA) or target sequence refers to a polynucleotide that is substantially complementary to a contiguous portion of the mRNA of interest or target sequence (e.g., an mRNA encoding a target gene). For example, a polynucleotide is complementary to at least a part of a target RNA if the sequence is substantially complementary to a non-interrupted portion of an mRNA encoding a target gene.

Accordingly, in some embodiments, the antisense strand polynucleotides disclosed herein are fully complementary to the target gene sequence. In other embodiments, the antisense polynucleotides disclosed herein are substantially complementary to the target sequence and comprise a contiguous nucleotide sequence which is at least 80% complementary over its entire length to the equivalent region of the nucleotide sequence of the target sequence, such as about 85%, about 90%, about 91%, about 92%, about 93%, about 94%, about 95%, about 96%, about 97%, about 98%, or about 99% complementary.

Exemplary target genes include, for example, adrenoceptor beta 1 (ADRB1); calcium voltage-gated channel subunit alpha1 C (CACNA1C); calcium voltage-gated channel subunit alpha1 G (CACNA1G) (T type calcium cchannel); angiotensin II receptor type 1 (AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); or Phosphodiesterase 1 (PDE1).

Additional exemplary target genes also include, for example, myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); Double Homeobox 4 (DUX4), dystrophy myotonic protein kinase (DMPK), glycogen synthase 1 (GYS1), survival of motor neuron 1 (SMN1), alpha-glucosidase (GAA), advanced glycosylation end-product specific receptor (AGER), mucin 5AC, oligomeric mucus/gel-forming (MUC5AC) and signal transducer and activator of transcription 6 (STAT6).

As used herein, “adrenoceptor beta 1,” used interchangeably with the term “ADRB1,” refers to a member of the adrenergic receptor family. The adrenergic receptors are a prototypic family of guanine nucleotide binding regulatory protein-coupled receptors that mediate the physiological effects of the hormone epinephrine and the neurotransmitter norepinephrine. Beta-1 adrenoceptors are predominately located in the heart. Specific polymorphisms in this gene have been shown to affect the resting heart rate and can be involved in heart failure. ADRB1 is also known as ADRB1R, beta-1 adrenergic receptor, B1AR, BETA1AR, FNSS2, or RHR.

An exemplary sequence of a human ADRB1 mRNA transcript can be found at, for example, GenBank Accession No. GI: 1653960731 (NM_000684.3; SEQ ID NO:1; reverse complement, SEQ ID NO: 5). The sequence of mouse ADRB1 mRNA can be found at, for example, GenBank Accession No. GI: 1693744501 (NM_007419.3; SEQ ID NO:2; reverse complement, SEQ ID NO: 6). The sequence of rat ADRB1 mRNA can be found at, for example, GenBank Accession No. GI: 6978458 (NM_012701.1; SEQ ID NO:3; reverse complement, SEQ ID NO: 7). The sequence of Macaca mulatta ADRB1 mRNA can be found at, for example, GenBank Accession No. GI: 577861029 (NM_001289866.1; SEQ ID NO: 4; reverse complement, SEQ ID NO: 8). The sequence of Macaca fascicularis ADRB1 mRNA can be found at, for example, GenBank Accession No. GI: 985482105 (NM_001319353.1; SEQ ID NO: 9; reverse complement, SEQ ID NO: 10).

Additional examples of ADRB1 mRNA sequences are readily available through publicly available databases, e.g., GenBank, UniProt, OMIM, UCSC Genome Browser, and the Macaca genome project web site.

Further information on ADRB1 can be found, for example, at www.ncbi.nlm.nih.gov/gene/?term=ADRB1.

The entire contents of each of the foregoing GenBank Accession numbers and the Gene database numbers are incorporated herein by reference as of the date of filing this application.

The term ADRB1, as used herein, also refers to variations of the ADRB1 gene including variants provided in the SNP database. Numerous sequence variations within the ADRB1 gene have been identified and may be found at, for example, NCBI dbSNP and UniProt (see, e.g., www.ncbi.nlm.nih.gov/snp/?term=ADRB1, the entire contents of which is incorporated herein by reference as of the date of filing this application.

In one embodiment, the target gene is calcium voltage-gated channel subunit alpha1 C (CACNA1C).

As used herein, “calcium voltage-gated channel subunit alpha1 C,” used interchangeably with the term “CACNA1C,” refers to an alpha-1 subunit of a voltage-dependent calcium channel. Calcium channels mediate the influx of calcium ions into the cell upon membrane polarization. The alpha-1 subunit consists of 24 transmembrane segments and forms the pore through which ions pass into the cell. The calcium channel consists of a complex of alpha-1, alpha-2/delta, beta, and gamma subunits in a 1:1:1:1 ratio. There are multiple isoforms of each of these proteins, either encoded by different genes or the result of alternative splicing of transcripts. The protein encoded by this gene binds to and is inhibited by dihydropyridine. CACNA1C is also known as calcium channel, voltage-dependent, L type, alpha 1C subunit; voltage-dependent L-type calcium channel subunit alpha-1C; voltage-gated L-type calcium channel Cav1.2 alpha 1 subunit, splice variant 10; calcium channel, L type, alpha-1 polypeptide, isoform 1, cardiac muscle; calcium channel, cardic dihydropyridine-sensitive, alpha-1 subunit; voltage-dependent L-type Ca2+ channel alpha 1 subunit; voltage-gated calcium channel subunit alpha CaV1.2; DHPR, alpha-1 subunit; CACH2, CACN2, CACNL1A1, CCHL1A1, CaV1.2, LQT8, TS, or TS. LQT8

An exemplary sequence of a human CACNA1C mRNA transcript can be found at, for example, GenBank Accession No. GI: 1890333913 (NM_199460.4; SEQ ID NO: 11; reverse complement, SEQ ID NO: 12). The sequence of mouse CACNA1C mRNA can be found at, for example, GenBank Accession No. GI: 594140631 (NM_009781.4; SEQ ID NO:13; reverse complement, SEQ ID NO: 14). The sequence of rat CACNA1C mRNA can be found at, for example, GenBank Accession No. GI: 158186632 (NM_012517.2; SEQ ID NO:15; reverse complement, SEQ ID NO: 16). The sequence of Macaca mulatta CACNA1C mRNA can be found at, for example, GenBank Accession No. GI: 1622843324 (XM_028829106.1; SEQ ID NO: 17; reverse complement, SEQ ID NO: 18).

Additional examples of CACNA1C mRNA sequences are readily available through publicly available databases, e.g., GenBank, UniProt, OMIM, UCSC Genome Browser, and the Macaca genome project website.

Further information on CACNA1C can be found, for example, at www.ncbi.nlm.nih.gov/gene/?term=CACNA1C.

The entire contents of each of the foregoing GenBank Accession numbers and the Gene database numbers are incorporated herein by reference as of the date of filing this application.

The term CACNA1C, as used herein, also refers to variations of the CACNA1C gene including variants provided in the SNP database. Numerous sequence variations within the CACNA1C gene have been identified and may be found at, for example, NCBI dbSNP and UniProt (see, e.g., www.ncbi.nlm.nih.gov/gene/?term=CACNA1C, the entire contents of which is incorporated herein by reference as of the date of filing this application.

As used herein, “calcium voltage-gated channel subunit alpha1 G,” used interchangeably with the term “CACNA1G,” refers to a T-type, low-voltage activated calcium channel. Voltage-sensitive calcium channels mediate the entry of calcium ions into excitable cells, and are also involved in a variety of calcium-dependent processes, including muscle contraction, hormone or neurotransmitter release, gene expression, cell motility, cell division, and cell death. The T-type channels generate currents that are both transient, owing to fast inactivation, and tiny, owing to small conductance. T-type channels are thought to be involved in pacemaker activity, low-threshold calcium spikes, neuronal oscillations and resonance, and rebound burst firing. CACNA1G is also known as calcium channel, voltage-dependent, T type, alpha 1G subunit; voltage-dependent T-type calcium channel subunit alpha-1G; voltage-gated calcium channel subunit alpha Cav3.1; NBR13; Cav3.1c; Ca(V)T.1; KIAA1123; SCA42ND; or SCA42.

An exemplary sequence of a human CACNA1G mRNA transcript can be found at, for example, GenBank Accession No. GI: 1519244109 (NM_018896.5; SEQ ID NO: 21; reverse complement, SEQ ID NO: 22). The sequence of mouse CACNA1G mRNA can be found at, for example, GenBank Accession No. GI: 295444826 (NM_009783.3; SEQ ID NO: 23; reverse complement, SEQ ID NO: 24). The sequence of rat CACNA1G mRNA can be found at, for example, GenBank Accession No. GI: 1995160279 (NM_001308302.2; SEQ ID NO: 25; reverse complement, SEQ ID NO: 26). The sequence of Macaca mulatta CACNA1G mRNA can be found at, for example, GenBank Accession No. GI: 1622879013 (XM_015119270.2; SEQ ID NO: 27; reverse complement, SEQ ID NO: 28). The sequence of Macaca fascicularis CACNA1G mRNA can be found at, for example, GenBank Accession No. GI: 982305044 (XM_005583707.2; SEQ ID NO: 29; reverse complement, SEQ ID NO: 30).

Additional exemplary examples of CACNA1G mRNA sequences are readily available through publicly available databases, e.g., GenBank, UniProt, OMIM, UCSC Genome Browser, and the Macaca genome project website.

Further information on CACNA1G can be found, for example, at www.ncbi.nlm.nih.gov/gene/?term=CACNA1G.

The entire contents of each of the foregoing GenBank Accession numbers and the Gene database numbers are incorporated herein by reference as of the date of filing this application.

The term CACNA1G, as used herein, also refers to variations of the CACNA1G gene including variants provided in the SNP database. Numerous sequence variations within the CACNA1G gene have been identified and may be found at, for example, NCBI dbSNP and UniProt (see, e.g., www.ncbi.nlm.nih.gov/gene/?term=CACNA1G, the entire contents of which is incorporated herein by reference as of the date of filing this application.

As used herein, “angiotensin II receptor type 1,” used interchangeably with the term “AGTR1,” refers to a receptor for the vasoconstricting peptide angiotensin II. Angiotensin II is a potent vasopressor hormone and a primary regulator of aldosterone secretion. AGTR1 is activated by angiotensin II. The activated receptor in turn couples to G protein and, thus, activates phospholipase C and increases the cytosolic Ca2+ concentrations, which in turn triggers cellular responses such as stimulation of protein kinase C. AGTR1 plays an integral role in blood pressure control, and is implicated in the pathogenesis of hypertension. AGTR1 is also known as angiotensin receptor 1B, AT1, AT2R1, AGTR1A, AT2R1B, AGTR1B, HAT1R, AG2S, AT1B, AT2R1A, AT1AR, AT1BR, or AT1R.

An exemplary sequence of a human AGTR1 mRNA transcript can be found at, for example, GenBank Accession No. GI: 1820101583 (NM_000685.5; SEQ ID NO: 31; reverse complement, SEQ ID NO: 32). The sequence of mouse AGTR1 mRNA can be found at, for example, GenBank Accession No. GI: 158937294 (NM_177322.3; SEQ ID NO: 33; reverse complement, SEQ ID NO: 34). The sequence of rat AGTR1 mRNA can be found at, for example, GenBank Accession No. GI: 140969764 (NM_030985.4; SEQ ID NO: 35; reverse complement, SEQ ID NO: 36). The sequence of Macaca mulatta AGTR1 mRNA can be found at, for example, GenBank Accession No. GI: 1622904093 (XM_028843763.1; SEQ ID NO: 37; reverse complement, SEQ ID NO: 38). The sequence of Macaca fascicularis AGTR1 mRNA can be found at, for example, GenBank Accession No. GI: 544411901 (XM_005546040.1; SEQ ID NO: 39; reverse complement, SEQ ID NO: 40).

Additional examples of AGTR1 mRNA sequences are readily available through publicly available databases, e.g., GenBank, UniProt, OMIM, UCSC Genome Browser, and the Macaca genome project website.

Further information on AGTR1 can be found, for example, at www.ncbi.nlm.nih.gov/gene/?term=AGTR1.

The entire contents of each of the foregoing GenBank Accession numbers and the Gene database numbers are incorporated herein by reference as of the date of filing this application.

The term AGTR1, as used herein, also refers to variations of the AGTR1 gene including variants provided in the SNP database. Numerous sequence variations within the AGTR1 gene have been identified and may be found at, for example, NCBI dbSNP and UniProt (see, e.g., www.ncbi.nlm.nih.gov/gene/?term=AGTR1, the entire contents of which is incorporated herein by reference as of the date of filing this application.

As used herein, “Sodium Voltage-Gated Channel Alpha Subunit 2,” used interchangeably with the term “SCN2A,” refers to a member of the voltage-gated sodium channel family. Voltage-gated sodium channels are transmembrane glycoprotein complexes composed of a large alpha subunit with four repeat domains, each of which is composed of six membrane-spanning segments, and one or more regulatory beta subunits. Voltage-gated sodium channels function in the generation and propagation of action potentials in neurons and muscle. Specifically, SCN2A permits the sodium influx from the extracellular space into the cytosol after depolarization of the nerve membrane. Allelic variants of SCN2A are associated with seizure disorders and autism spectrum disorders. SCN2A is also known as Nav1.2, HBSCII, SCN2A1, SCN2A2, HBSCI, EIEE11, BFIC3, BFIS3, BFNIS, DEE11, EA9, or HBA.

An exemplary sequence of a human SCN2A mRNA transcript can be found at, for example, GenBank Accession No. GI: 1697699196 (NM_021007.3; SEQ ID NO: 41; reverse complement, SEQ ID NO: 42). The sequence of mouse SCN2A mRNA can be found at, for example, GenBank Accession No. GI: 1114439824 (NM_001099298.3; SEQ ID NO: 43; reverse complement, SEQ ID NO: 44). The sequence of rat SCN2A mRNA can be found at, for example, GenBank Accession No. GI: 1937915892 (NM 012647.2; SEQ ID NO: 45; reverse complement, SEQ ID NO: 46). The sequence of Macaca mulatta SCN2A mRNA can be found at, for example, GenBank Accession No. GI: 1622850108 (XM_001100368.4; SEQ ID NO: 47; reverse complement, SEQ ID NO: 48). The sequence of Macaca fascicularis SCN2A mRNA can be found at, for example, GenBank Accession No. GI: 544475515 (XM_005573351.1; SEQ ID NO: 49; reverse complement, SEQ ID NO: 50).

Additional examples of SCN2A mRNA sequences are readily available through publicly available databases, e.g., GenBank, UniProt, OMIM, UCSC Genome Browser, and the Macaca genome project website.

Further information on SCN2A can be found, for example, at www.ncbi.nlm.nih.gov/gene/?term=SCN2A.

The entire contents of each of the foregoing GenBank Accession numbers and the Gene database numbers are incorporated herein by reference as of the date of filing this application.

The term SCN2A, as used herein, also refers to variations of the SCN2A gene including variants provided in the SNP database. Numerous sequence variations within the SCN2A gene have been identified and may be found at, for example, NCBI dbSNP and UniProt (see, e.g., www.ncbi.nlm.nih.gov/gene/?term=SCN2A, the entire contents of which is incorporated herein by reference as of the date of filing this application.

As used herein, “Hyperpolarization Activated Cyclic Nucleotide Gated Potassium Channel 1,” used interchangeably with the term “HCN1,” refers to a member of the hyperpolarization-activated cyclic nucleotide-gated (HCN) channel family. These channels are primarily expressed in the heart and in the central and peripheral nervous systems. HCN channels mediate rhythmic electrical activity of cardiac pacemaker cells, and in neurons play important roles in setting resting membrane potentials, dendritic integration, neuronal pacemaking, and establishing action potential threshold. The HCN1 protein can homodimerize or heterodimerize with other pore-forming subunits to form a potassium channel. HCN1 is also known as potassium channel 1, BCNG-1, HAC-2, BCNG1, Potassium/Sodium Hyperpolarization-Activated Cyclic Nucleotide-Gated Channel 1; Brain Cyclic Nucleotide-Gated Channel 1; Hyperpolarization Activated Cyclic Nucleotide-Gated Potassium Channel 1; GEFSP10, EIEE24, or DEE24.

An exemplary sequence of a human HCN1 mRNA transcript can be found at, for example, GenBank Accession No. GI: 1519313076 (NM_021072.4; SEQ ID NO: 51; reverse complement, SEQ ID NO: 52). The sequence of mouse HCN1 mRNA can be found at, for example, GenBank Accession No. GI: 283837798 (NM_010408.3; SEQ ID NO: 53; reverse complement, SEQ ID NO: 54). The sequence of rat HCN1 mRNA can be found at, for example, GenBank Accession No. GI: 2000186052 (NM_053375.2; SEQ ID NO: 55; reverse complement, SEQ ID NO: 56). The sequence of Macaca mulatta HCN1 mRNA can be found at, for example, GenBank Accession No. GI: 1622944535 (XM_015140004.2; SEQ ID NO: 57; reverse complement, SEQ ID NO: 58). The sequence of Macaca fascicularis HCN1 mRNA can be found at, for example, GenBank Accession No. GI: 982252681 (XM_005556858.2; SEQ ID NO: 59; reverse complement, SEQ ID NO: 60).

Additional examples of HCN1 mRNA sequences are readily available through publicly available databases, e.g., GenBank, UniProt, OMIM, UCSC Genome Browser, and the Macaca genome project website.

Further information on HCN1 can be found, for example, at www.ncbi.nlm.nih.gov/gene/?term=HCN1.

The entire contents of each of the foregoing GenBank Accession numbers and the Gene database numbers are incorporated herein by reference as of the date of filing this application.

The term HCN1, as used herein, also refers to variations of the HCN1 gene including variants provided in the SNP database. Numerous sequence variations within the HCN1 gene have been identified and may be found at, for example, NCBI dbSNP and UniProt (see, e.g., www.ncbi.nlm.nih.gov/gene/?term=HCN1, the entire contents of which is incorporated herein by reference as of the date of filing this application.

As used herein, “Hyperpolarization Activated Cyclic Nucleotide Gated Potassium Channel 4,” used interchangeably with the term “HCN4,” refers to a member of the hyperpolarization-activated cyclic nucleotide-gated (HCN) channel family. The HCN4 channel transports positively charged ions into heart muscle cells. This channel is located primarily in the sino-atrial (SA) node, which is an area of specialized cells in the heart that functions as a natural pacemaker. The HCN4 channel allows potassium and sodium ions to flow into cells of the SA node. This ion flow is often called the “pacemaker current” because it generates electrical impulses that start each heartbeat and is involved in maintaining a regular heart rhythm. HCN4 is also known as Potassium/Sodium Hyperpolarization-Activated Cyclic Nucleotide-Gated Channel 4, Hyperpolarization Activated Cyclic Nucleotide-Gated Potassium Channel 4, Hyperpolarization Activated Cyclic Nucleotide-Gated Cation Channel 4 or SSS2.

An exemplary sequence of a human HCN4 mRNA transcript can be found at, for example, GenBank Accession No. GI: 1519312820 (NM_005477.3; SEQ ID NO: 61; reverse complement, SEQ ID NO: 62). The sequence of mouse HCN4 mRNA can be found at, for example, GenBank Accession No. GI: 1686254400 (NM_001081192.3; SEQ ID NO: 63; reverse complement, SEQ ID NO: 64). The sequence of rat HCN4 mRNA can be found at, for example, GenBank Accession No. GI: 1937893976 (NM 021658.2; SEQ ID NO: 65; reverse complement, SEQ ID NO: 66). The sequence of Macaca mulatta HCN4 mRNA can be found at, for example, GenBank Accession No. GI: 1622953870 (XM_002804859.3; SEQ ID NO: 67; reverse complement, SEQ ID NO: 68). The sequence of Macaca fascicularis HCN4 mRNA can be found at, for example, GenBank Accession No. GI: 982258526 (XM_005559993.2; SEQ ID NO: 69; reverse complement, SEQ ID NO: 70).

Additional examples of HCN4 mRNA sequences are readily available through publicly available databases, e.g., GenBank, UniProt, OMIM, UCSC Genome Browser, and the Macaca genome project website.

Further information on HCN4 can be found, for example, at www.ncbi.nlm.nih.gov/gene/?term=HCN4.

The entire contents of each of the foregoing GenBank Accession numbers and the Gene database numbers are incorporated herein by reference as of the date of filing this application.

The term HCN4, as used herein, also refers to variations of the HCN4 gene including variants provided in the SNP database. Numerous sequence variations within the HCN4 gene have been identified and may be found at, for example, NCBI dbSNP and UniProt (see, e.g., www.ncbi.nlm.nih.gov/gene/?term=HCN4, the entire contents of which is incorporated herein by reference as of the date of filing this application.

As used herein, “Hyperpolarization Activated Cyclic Nucleotide Gated Potassium Channel 3,” used interchangeably with the term “HCN3,” refers to a member of the hyperpolarization-activated cyclic nucleotide-gated (HCN) channel family. A study conducted in the mouse suggested that HCN3 channels might be involved in the regulation of the circadian system. HCN3 channels have also been reported to be present in the intergeniculate leaflet of the hypothalamus. HCN3 is also known as Potassium/Sodium Hyperpolarization-Activated Cyclic Nucleotide-Gated Channel 3, Hyperpolarization Activated Cyclic Nucleotide-Gated Potassium Channel 3, or KIAA1535.

An exemplary sequence of a human HCN3 mRNA transcript can be found at, for example, GenBank Accession No. GI: 1519312303 (NM_020897.3; SEQ ID NO: 71; reverse complement, SEQ ID NO: 72). The sequence of mouse HCN3 mRNA can be found at, for example, GenBank Accession No. GI: 6680190 (NM_008227.1; SEQ ID NO: 73; reverse complement, SEQ ID NO: 74). The sequence of rat HCN3 mRNA can be found at, for example, GenBank Accession No. GI: 16758501 (NM_053685.1; SEQ ID NO: 75; reverse complement, SEQ ID NO: 76). The sequence of Macaca mulatta HCN3 mRNA can be found at, for example, GenBank Accession No. GI: 1622829938 (XM_001115891.4; SEQ ID NO: 77; reverse complement, SEQ ID NO: 78). The sequence of Macaca fascicularis HCN3 mRNA can be found at, for example, GenBank Accession No. GI: 982225310 (XM_005541549.2; SEQ ID NO: 79; reverse complement, SEQ ID NO: 80).

Additional examples of HCN3 mRNA sequences are readily available through publicly available databases, e.g., GenBank, UniProt, OMIM, UCSC Genome Browser, and the Macaca genome project website.

Further information on HCN3 can be found, for example, at www.ncbi.nlm.nih.gov/gene/?term=HCN3.

The entire contents of each of the foregoing GenBank Accession numbers and the Gene database numbers are incorporated herein by reference as of the date of filing this application.

The term HCN3, as used herein, also refers to variations of the HCN3 gene including variants provided in the SNP database. Numerous sequence variations within the HCN3 gene have been identified and may be found at, for example, NCBI dbSNP and UniProt (see, e.g., www.ncbi.nlm.nih.gov/gene/?term=HCN3, the entire contents of which is incorporated herein by reference as of the date of filing this application.

As used herein, “Potassium Voltage-Gated Channel Subfamily A Member 5,” used interchangeably with the term “KCNA5,” refers to a member of the voltage-gated potassium channel family. The Voltage-gated potassium channels mediate transmembrane potassium transport in excitable membranes. These channels form tetrameric potassium-selective channels through which potassium ions pass in accordance with their electrochemical gradient, and alternate between opened and closed conformations in response to the voltage difference across the membrane. KCNA5 contains six membrane-spanning domains with a shaker-type repeat in the fourth segment. It belongs to the delayed rectifier class, the function of which could restore the resting membrane potential of beta cells after depolarization and thereby contribute to the regulation of insulin secretion. KCNA5 is also known as HPCN1, HK2, Potassium Voltage-Gated Channel, Shaker-Related Subfamily, Member 5; Voltage-Gated Potassium Channel Subunit Kv1.5; Voltage-Gated Potassium Channel HK2; Kv1.5; Insulinoma And Islet Potassium Channel; Cardiac Potassium Channel; Potassium Channel 1; ATFB7, HCK1 or PCN1.

An exemplary sequence of a human KCNA5 mRNA transcript can be found at, for example, GenBank Accession No. GI: 1653961222 (NM_002234.4; SEQ ID NO: 81; reverse complement, SEQ ID NO: 82). The sequence of mouse KCNA5 mRNA can be found at, for example, GenBank Accession No. GI: 158937280 (NM_145983.2; SEQ ID NO: 83; reverse complement, SEQ ID NO: 84). The sequence of rat KCNA5 mRNA can be found at, for example, GenBank Accession No. GI: 6981117 (NM_012972.1; SEQ ID NO: 85; reverse complement, SEQ ID NO: 86). The sequence of Macaca mulatta KCNA5 mRNA can be found at, for example, GenBank Accession No. GI: 1622843572 (XM_001102294.4; SEQ ID NO: 87; reverse complement, SEQ ID NO: 88). The sequence of Macaca fascicularis KCNA5 mRNA can be found at, for example, GenBank Accession No. GI: 982279162 (XM_005569870.2; SEQ ID NO: 89; reverse complement, SEQ ID NO: 90).

Additional examples of KCNA5 mRNA sequences are readily available through publicly available databases, e.g., GenBank, UniProt, OMIM, UCSC Genome Browser, and the Macaca genome project website.

Further information on KCNA5 can be found, for example, at www.ncbi.nlm.nih.gov/gene/?term=KCNA5.

The entire contents of each of the foregoing GenBank Accession numbers and the Gene database numbers are incorporated herein by reference as of the date of filing this application.

The term KCNA5, as used herein, also refers to variations of the KCNA5 gene including variants provided in the SNP database. Numerous sequence variations within the KCNA5 gene have been identified and may be found at, for example, NCBI dbSNP and UniProt (see, e.g., www.ncbi.nlm.nih.gov/gene/?term=KCNA5, the entire contents of which is incorporated herein by reference as of the date of filing this application.

As used herein, “Potassium Inwardly Rectifying Channel Subfamily J Member 3,” used interchangeably with the term “KCNJ3,” refers to an integral membrane protein and an inward-rectifier type potassium channel. The inward-rectifier type potassium channels have a greater tendency to allow potassium to flow into a cell rather than out of a cell. This asymmetry in potassium ion conductance plays a key role in the excitability of muscle cells and neurons. KCNJ3 is controlled by G-proteins and plays an important role in regulating heartbeat. It associates with three other G-protein-activated potassium channels to form a heteromultimeric pore-forming complex, which also couples to neurotransmitter receptors in the brain. These multimeric G-protein-gated inwardly-rectifying potassium (GIRK) channels have a wide range of physiological roles, including the regulation of heartbeat, reward mechanisms, learning and memory functions, blood platelet aggregation, insulin secretion, and lipid metabolism. KCNJ3 is also known as GIRK1, G Protein-Activated Inward Rectifier Potassium Channel 1, KGA; Potassium Channel, Inwardly Rectifying Subfamily J Member 3; Inward Rectifier K(+) Channel Kir3.1; or Potassium Inwardly-Rectifying Channel Subfamily J Member 3 Splice Variant 1e.

An exemplary sequence of a human KCNJ3 mRNA transcript can be found at, for example, GenBank Accession No. GI: 1519246021 (NM_002239.4; SEQ ID NO: 91; reverse complement, SEQ ID NO: 92). The sequence of mouse KCNJ3 mRNA can be found at, for example, GenBank Accession No. GI: 756398330 (NM_008426.2; SEQ ID NO: 93; reverse complement, SEQ ID NO: 94). The sequence of rat KCNJ3 mRNA can be found at, for example, GenBank Accession No. GI: 148747456 (NM_031610.3; SEQ ID NO: 95; reverse complement, SEQ ID NO: 96). The sequence of Macaca mulatta KCNJ3 mRNA can be found at, for example, GenBank Accession No. GI: 387849010 (NM_001261696.1; SEQ ID NO: 97; reverse complement, SEQ ID NO: 98). The sequence of Macaca fascicularis KCNJ3 mRNA can be found at, for example, GenBank Accession No. GI: 982285759 (XM_005573205.2; SEQ ID NO: 99; reverse complement, SEQ ID NO: 100).

Additional examples of KCNJ3 mRNA sequences are readily available through publicly available databases, e.g., GenBank, UniProt, OMIM, UCSC Genome Browser, and the Macaca genome project website.

Further information on KCNJ3 can be found, for example, at www.ncbi.nlm.nih.gov/gene/?term=KCNJ3.

The entire contents of each of the foregoing GenBank Accession numbers and the Gene database numbers are incorporated herein by reference as of the date of filing this application.

The term KCNJ3, as used herein, also refers to variations of the KCNJ3 gene including variants provided in the SNP database. Numerous sequence variations within the KCNJ3 gene have been identified and may be found at, for example, NCBI dbSNP and UniProt (see, e.g., www.ncbi.nlm.nih.gov/gene/?term=KCNAJ3, the entire contents of which is incorporated herein by reference as of the date of filing this application.

As used herein, “Potassium Inwardly Rectifying Channel Subfamily J Member 4,” used interchangeably with the term “KCNJ4,” refers to an integral membrane protein and inward-rectifier type potassium channel. The inward-rectifier type potassium channels have a greater tendency to allow potassium to flow into a cell rather than out of a cell. This asymmetry in potassium ion conductance plays a key role in the excitability of muscle cells and neurons. KCNJ4 can tetramerize to form functional inwardly rectifying channels, in which each monomer contains two transmembrane helix domains, an ion-selective P-loop, and cytoplasmic N- and C-terminal domains. The distribution of KCNJ4 is predominantly focused in both heart and brain, especially in the cardiac myocytes and forebrain region. KCNJ4 may play important roles in the regulation of resting membrane potential, cellular excitability and potassium homeostasis in the nervous system and various peripheral tissues. KCNJ4 is also known as HIRK2, HRK1, IRK3, HIR, Kir2.3, inward rectifier potassium channel 4; Inward Rectifier K(+) Channel Kir2.3; Potassium Voltage-Gated Channel Subfamily J Member 4; Hippocampal Inward Rectifier Potassium Channel; or Hippocampal Inward Rectifier.

An exemplary sequence of a human KCNJ4 mRNA transcript can be found at, for example, GenBank Accession No. GI: 1732746379 (NM_152868.3; SEQ ID NO: 101; reverse complement, SEQ ID NO: 102). The sequence of mouse KCNJ4 mRNA can be found at, for example, GenBank Accession No. GI: 1720383422 (XM_006520486.4; SEQ ID NO: 103; reverse complement, SEQ ID NO: 104). The sequence of rat KCNJ4 mRNA can be found at, for example, GenBank Accession No. GI: 1937901561 (NM 053870.3; SEQ ID NO: 105; reverse complement, SEQ ID NO: 106). The sequence of Macaca mulatta KCNJ4 mRNA can be found at, for example, GenBank Accession No. GI: 1622838042 (XM_015150354.2; SEQ ID NO: 107; reverse complement, SEQ ID NO: 108). The sequence of Macaca fascicularis KCNJ4 mRNA can be found at, for example, GenBank Accession No. GI: 544461851 (XM_005567299.1; SEQ ID NO: 109; reverse complement, SEQ ID NO: 110).

Additional examples of KCNJ4 mRNA sequences are readily available through publicly available databases, e.g., GenBank, UniProt, OMIM, UCSC Genome Browser, and the Macaca genome project website.

Further information on KCNJ4 can be found, for example, at www.ncbi.nlm.nih.gov/gene/?term=KCNJ4.

The entire contents of each of the foregoing GenBank Accession numbers and the Gene database numbers are incorporated herein by reference as of the date of filing this application.

The term KCNJ4, as used herein, also refers to variations of the KCNJ4 gene including variants provided in the SNP database. Numerous sequence variations within the KCNJ4 gene have been identified and may be found at, for example, NCBI dbSNP and UniProt (see, e.g., www.ncbi.nlm.nih.gov/gene/?term=KCNAJ4, the entire contents of which is incorporated herein by reference as of the date of filing this application. As used herein, “Phosphodiesterase 1,” used interchangeably with the term “PDE1,” refers to a member of the cyclic nucleotide phosphodiesterases families. Cyclic nucleotide phosphodiesterases (PDEs) are superfamily of enzymes that regulate the spatial and temporal relationship of second messenger signaling in the cellular system. Among the 11 different families of PDEs, phosphodiesterase 1 (PDE1) sub-family of enzymes hydrolyze both 3′,5′-cyclic adenosine monophosphate (cAMP) and 3′,5′-cyclic guanosine monophosphate (cGMP) in a mutually competitive manner. The catalytic activity of PDE1 is stimulated by their binding to Ca2+/calmodulin (CaM), resulting in the integration of Ca2+ and cyclic nucleotide-mediated signaling in various diseases. The PDE1 family includes three subtypes, PDE1A, PDE1B and PDE1C, which differ for their relative affinities for cAMP and cGMP. These isoforms are differentially expressed throughout the body, including the cardiovascular, central nervous system and other organs. Thus, PDE1 enzymes play a critical role in the pathophysiology of diseases through the fundamental regulation of cAMP and cGMP signaling. PDE1 is also known as Calcium/Calmodulin-Dependent 3′,5′-Cyclic Nucleotide Phosphodiesterase 1; Calcium/Calmodulin-Stimulated Cyclic Nucleotide Phosphodiesterase; CAM-PDE 1, HSPDE1, HCAM1, or EC 3.1.4.

An exemplary sequence of a human PDE1 mRNA transcript can be found at, for example, GenBank Accession No. GI: 2062580163 (NM_005019.7; SEQ ID NO: 111; reverse complement, SEQ ID NO: 112). The sequence of mouse PDE1 mRNA can be found at, for example, GenBank Accession No. GI: 227330628 (NM_001159582.1; SEQ ID NO: 113; reverse complement, SEQ ID NO: 114). The sequence of rat PDE1 mRNA can be found at, for example, GenBank Accession No. GI: 13540702 (NM_030871.1; SEQ ID NO: 115; reverse complement, SEQ ID NO: 116). The sequence of Macaca mulatta PDE1 mRNA can be found at, for example, GenBank Accession No. GI: 383872283 (NM_001257584.1; SEQ ID NO: 117; reverse complement, SEQ ID NO: 118). The sequence of Macaca fascicularis PDE1 mRNA can be found at, for example, GenBank Accession No. GI: 982286500 (XR_001483985.1; SEQ ID NO: 119; reverse complement, SEQ ID NO: 120).

Additional examples of PDE1 mRNA sequences are readily available through publicly available databases, e.g., GenBank, UniProt, OMIM, UCSC Genome Browser, and the Macaca genome project website.

Further information on PDE1 can be found, for example, at www.ncbi.nlm.nih.gov/gene/?term=PDE1.

The entire contents of each of the foregoing GenBank Accession numbers and the Gene database numbers are incorporated herein by reference as of the date of filing this application.

The term PDE1, as used herein, also refers to variations of the PDE1 gene including variants provided in the SNP database. Numerous sequence variations within the PDE1 gene have been identified and may be found at, for example, NCBI dbSNP and UniProt (see, e.g., www.ncbi.nlm.nih.gov/gene/?term=PDE1, the entire contents of which is incorporated herein by reference as of the date of filing this application.

As used herein, “myostatin,” used interchangeably with the term “MSTN,” refers to a secreted ligand of the TGF-beta (transforming growth factor-beta) superfamily of proteins. Ligands of this family bind various TGF-beta receptors leading to recruitment and activation of SMAD family transcription factors that regulate gene expression. The encoded preproprotein is proteolytically processed to generate each subunit of the disulfide-linked homodimer. This protein negatively regulates skeletal muscle cell proliferation and differentiation. Mutations in this gene are associated with increased skeletal muscle mass in humans and other mammals. Myostatin is also known as GDF8, Growth/Differentiation Factor 8, or MSLHP.

The sequence of a human myostatin mRNA transcript can be found at, for example, GenBank Accession No. GI: 1653961810 (NM_005259.3; SEQ ID NO:221; reverse complement, SEQ ID NO: 222). The sequence of mouse myostatin mRNA can be found at, for example, GenBank Accession No. GI: 922959927 (NM_010834.3; SEQ ID NO:223; reverse complement, SEQ ID NO: 224). The sequence of rat myostatin mRNA can be found at, for example, GenBank Accession No. GI: 9506906 (NM_019151.1; SEQ ID NO:225; reverse complement, SEQ ID NO: 226). The sequence of Macaca fascicularis myostatin mRNA can be found at, for example, GenBank Accession No. NM_001287623.1; SEQ ID NO: 227; reverse complement, SEQ ID NO: 228. The sequence of Macaca mulatta myostatin mRNA can be found at, for example, GenBank Accession No. GI: 121583757 (NM_001080119.1; SEQ ID NO: 229; reverse complement, SEQ ID NO: 230).

Additional examples of myostatin mRNA sequences are readily available through publicly available databases, e.g., GenBank, UniProt, OMIM, and the Macaca genome project web site.

Further information on myostatin can be found, for example, at www.ncbi.nlm.nih.gov/gene/?term=myostatin.

The entire contents of each of the foregoing GenBank Accession numbers and the Gene database numbers are incorporated herein by reference as of the date of filing this application.

The term myostatin, as used herein, also refers to variations of the myostatin gene including variants provided in the SNP database. Numerous sequence variations within the myostatin gene have been identified and may be found at, for example, NCBI dbSNP and UniProt (see, e.g., www.ncbi.nlm.nih.gov/snp/?term=myostatin, the entire contents of which is incorporated herein by reference as of the date of filing this application.

As used herein, “Cholinergic Receptor Nicotinic Alpha 1 Subunit,” used interchangeably with the term “CHRNA1,” refers to an alpha subunit of the muscle acetylcholine receptor (AChR). The muscle acetylcholine receptor consists of 5 subunits of 4 different types: 2 alpha subunits and 1 each of the beta, gamma, and delta subunits. This protein plays a role in acetylcholine binding/channel gating. After binding acetylcholine, the AChR responds by an extensive change in conformation that affects all subunits and leads to opening of an ion-conducting channel across the plasma membrane. CHRNA1 is associated with diseases associated such as Myasthenic Syndrome. CHRNA1 is also known as Cholinergic Receptor, Nicotinic, Alpha Polypeptide 1; Acetylcholine Receptor, Nicotinic, Alpha 1 (Muscle); ACHRA; CHRNA; Muscle Nicotinic Acetylcholine Receptor; CMS1A, CMS1B, CMS2A, FCCMS, SCCMS, or ACHRD.

The sequence of a human CHRNA1 mRNA transcript can be found at, for example, GenBank Accession No. GI: 1676317412 (NM_001039523.3; SEQ ID NO:231; reverse complement, SEQ ID NO: 232). The sequence of mouse CHRNA1 mRNA can be found at, for example, GenBank Accession No. GI: 425905338 (NM_007389.5; SEQ ID NO:233; reverse complement, SEQ ID NO: 234). The sequence of rat CHRNA1 mRNA can be found at, for example, GenBank Accession No. GI: 1937369362 (NM 024485.2; SEQ ID NO:235; reverse complement, SEQ ID NO: 236). The sequence of Macaca fascicularis CHRNA1 mRNA can be found at, for example, GenBank Accession No. GI: 982286285 (XM_015432377.1; SEQ ID NO: 237; reverse complement, SEQ ID NO: 238). The sequence of Macaca mulatta CHRNA1 mRNA can be found at, for example, GenBank Accession No. GI: 1622850381 (XM_001091711.4; SEQ ID NO: 239; reverse complement, SEQ ID NO: 240).

Additional examples of CHRNA1 mRNA sequences are readily available through publicly available databases, e.g., GenBank, UniProt, OMIM, and the Macaca genome project web site. Further information on CHRNA1 can be found, for example, at www.ncbi.nlm.nih.gov/gene/?term=CHRNA1.

The entire contents of each of the foregoing GenBank Accession numbers and the Gene database numbers are incorporated herein by reference as of the date of filing this application.

The term CHRNA1, as used herein, also refers to variations of the CHRNA1 gene including variants provided in the SNP database. Numerous sequence variations within the CHRNA1 gene have been identified and may be found at, for example, NCBI dbSNP and UniProt (see, e.g., www.ncbi.nlm.nih.gov/snp/?term=CHRNA1, the entire contents of which is incorporated herein by reference as of the date of filing this application.

As used herein, “Cholinergic Receptor Nicotinic Beta 1 Subunit,” used interchangeably with the term “CHRNB1,” refers to a beta subunit of the muscle acetylcholine receptor (AChR). The muscle acetylcholine receptor consists of 5 subunits of 4 different types: 2 alpha subunits and 1 each of the beta, gamma, and delta subunits. This protein plays a role in acetylcholine binding/channel gating. After binding acetylcholine, the AChR responds by an extensive change in conformation that affects all subunits and leads to opening of an ion-conducting channel across the plasma membrane. CHRNB1 is associated with diseases associated such as Myasthenic Syndrome. CHRNB1 is also known as Cholinergic Receptor, Nicotinic, Beta Polypeptide 1; Acetylcholine Receptor, Nicotinic, Beta 1 (Muscle); ACHRB; CHRNB; CMS1D, CMS2C, CMS2A, or SCCMS.

The sequence of a human CHRNB1 mRNA transcript can be found at, for example, GenBank Accession No. GI: 1519313560 (NM_000747.3; SEQ ID NO:241; reverse complement, SEQ ID NO: 242). The sequence of mouse CHRNB1 mRNA can be found at, for example, GenBank Accession No. GI: 160358781 (NM_009601.4; SEQ ID NO:243; reverse complement, SEQ ID NO: 244). The sequence of rat CHRNB1 mRNA can be found at, for example, GenBank Accession No. GI: 2048631755 (NM_001395118.1; SEQ ID NO:245; reverse complement, SEQ ID NO: 246). The sequence of Macaca fascicularis CHRNB1 mRNA can be found at, for example, GenBank Accession No. GI: 982302904 (XM_005582753.2; SEQ ID NO: 247; reverse complement, SEQ ID NO: 248). The sequence of Macaca mulatta CHRNB1 mRNA can be found at, for example, GenBank Accession No. GI: 1622877217 (XM_015118481.2; SEQ ID NO: 249; reverse complement, SEQ ID NO: 250).

Additional examples of CHRNB1 mRNA sequences are readily available through publicly available databases, e.g., GenBank, UniProt, OMIM, and the Macaca genome project web site. Further information on CHRNB1 can be found, for example, at www.ncbi.nlm.nih.gov/gene/?term=CHRNB1.

The entire contents of each of the foregoing GenBank Accession numbers and the Gene database numbers are incorporated herein by reference as of the date of filing this application.

The term CHRNB1, as used herein, also refers to variations of the CHRNB1 gene including variants provided in the SNP database. Numerous sequence variations within the CHRNB1 gene have been identified and may be found at, for example, NCBI dbSNP and UniProt (see, e.g., www.ncbi.nlm.nih.gov/snp/?term=CHRNB1, the entire contents of which is incorporated herein by reference as of the date of filing this application.

As used herein, “Cholinergic Receptor Nicotinic Delta Subunit,” used interchangeably with the term “CHRND,” refers to a delta subunit of the muscle acetylcholine receptor (AChR). The muscle acetylcholine receptor consists of 5 subunits of 4 different types: 2 alpha subunits and 1 each of the beta, gamma, and delta subunits. After binding acetylcholine, the AChR responds by an extensive change in conformation that affects all subunits and leads to opening of an ion-conducting channel across the plasma membrane. CHRND is associated with diseases associated such as Myasthenic Syndrome. CHRND is also known as ACHRD, Cholinergic Receptor, Nicotinic, Delta Polypeptide; Acetylcholine Receptor, Nicotinic, Delta (Muscle); CMS2A; CMS3A, CMS3B, CMS3C, FCCMS, or SCCMS.

The sequence of a human CHRND mRNA transcript can be found at, for example, GenBank Accession No. GI: 1519243557 (NM_000751.3; SEQ ID NO:251; reverse complement, SEQ ID NO: 252). The sequence of mouse CHRND mRNA can be found at, for example, GenBank Accession No. GI: 426214082 (NM_021600.3; SEQ ID NO:253; reverse complement, SEQ ID NO: 254). The sequence of rat CHRND mRNA can be found at, for example, GenBank Accession No. GI: 9506486 (NM 019298.1; SEQ ID NO:255; reverse complement, SEQ ID NO: 256). The sequence of Macaca fascicularis CHRND mRNA can be found at, for example, GenBank Accession No. GI: 982288086 (XM_005574618.2; SEQ ID NO: 257; reverse complement, SEQ ID NO: 258). The sequence of Macaca mulatta CHRND mRNA can be found at, for example, GenBank Accession No. GI: 1622852529 (XM_028831231.1; SEQ ID NO: 259; reverse complement, SEQ ID NO: 260).

Additional examples of CHRND mRNA sequences are readily available through publicly available databases, e.g., GenBank, UniProt, OMIM, and the Macaca genome project web site. Further information on CHRND can be found, for example, at www.ncbi.nlm.nih.gov/gene/?term=CHRND.

The entire contents of each of the foregoing GenBank Accession numbers and the Gene database numbers are incorporated herein by reference as of the date of filing this application.

The term CHRND, as used herein, also refers to variations of the CHRND gene including variants provided in the SNP database. Numerous sequence variations within the CHRND gene have been identified and may be found at, for example, NCBI dbSNP and UniProt (see, e.g., www.ncbi.nlm.nih.gov/snp/?term=CHRND, the entire contents of which is incorporated herein by reference as of the date of filing this application.

As used herein, “Cholinergic Receptor Nicotinic Epsilon Subunit,” used interchangeably with the term “CHRNE,” refers to a subunit of the acetylcholine receptor. Acetylcholine receptors at mature mammalian neuromuscular junctions are pentameric protein complexes composed of four subunits in the ratio of two alpha subunits to one beta, one epsilon, and one delta subunit. The acetylcholine receptor changes subunit composition shortly after birth when the epsilon subunit replaces the gamma subunit seen in embryonic receptors. Mutations in the epsilon subunit are associated with congenital myasthenic syndrome. CHRNE is also known as Cholinergic Receptor, Nicotinic, Epsilon; Acetylcholine Receptor, Nicotinic, Epsilon; ACHRE; CMS1D, CMS1E, CMS2A, CMS4A, CMS4B, CMS4C, FCCMS, or SCCMS.

The sequence of a human CHRNE mRNA transcript can be found at, for example, GenBank Accession No. GI: 1433531118 (NM_000080.4; SEQ ID NO: 261; reverse complement, SEQ ID NO: 262). The sequence of mouse CHRNE mRNA can be found at, for example, GenBank Accession No. GI: 6752949 (NM_009603.1; SEQ ID NO: 263; reverse complement, SEQ ID NO: 264). The sequence of rat CHRNE mRNA can be found at, for example, GenBank Accession No. GI: 8393128 (NM_017194.1; SEQ ID NO: 265; reverse complement, SEQ ID NO: 266). The sequence of Macaca fascicularis CHRNE mRNA can be found at, for example, GenBank Accession No. GI: 982302635 (XM_015437499.1; SEQ ID NO: 267; reverse complement, SEQ ID NO: 268). The sequence of Macaca mulatta CHRNE mRNA can be found at, for example, GenBank Accession No. GI: 1622876897 (XM_015118354.2; SEQ ID NO: 269; reverse complement, SEQ ID NO: 270).

Additional examples of CHRNE mRNA sequences are readily available through publicly available databases, e.g., GenBank, UniProt, OMIM, and the Macaca genome project web site. Further information on CHRNE can be found, for example, at www.ncbi.nlm.nih.gov/gene/?term=CHRNE.

The entire contents of each of the foregoing GenBank Accession numbers and the Gene database numbers are incorporated herein by reference as of the date of filing this application.

The term CHRNE, as used herein, also refers to variations of the CHRNE gene including variants provided in the SNP database. Numerous sequence variations within the CHRNE gene have been identified and may be found at, for example, NCBI dbSNP and UniProt (see, e.g., www.ncbi.nlm.nih.gov/snp/?term=CHRNE, the entire contents of which is incorporated herein by reference as of the date of filing this application.

As used herein, “Cholinergic Receptor Nicotinic Gamma Subunit,” used interchangeably with the term “CHRNG,” refers to a subunit of the acetylcholine receptor. The mammalian muscle-type acetylcholine receptor is a transmembrane pentameric glycoprotein with two alpha subunits, one beta, one delta, and one epsilon (in adult skeletal muscle) or gamma (in fetal and denervated muscle) subunit. This gene, which encodes the gamma subunit, is expressed prior to the thirty-third week of gestation in humans. The gamma subunit of the acetylcholine receptor plays a role in neuromuscular organogenesis and ligand binding and disruption of gamma subunit expression prevents the correct localization of the receptor in cell membranes. Mutations in the subunit are associated with congenital myasthenic syndrome. CHRNG is also known as Cholinergic Receptor, Nicotinic, Gamma; Acetylcholine Receptor, Nicotinic, Gamma; or ACHRG.

The sequence of a human CHRNG mRNA transcript can be found at, for example, GenBank Accession No. GI: 1441481359 (NM_005199.5; SEQ ID NO: 271; reverse complement, SEQ ID NO: 272). The sequence of mouse CHRNG mRNA can be found at, for example, GenBank Accession No. GI: 119964695 (NM_009604.3; SEQ ID NO: 273; reverse complement, SEQ ID NO: 274). The sequence of rat CHRNG mRNA can be found at, for example, GenBank Accession No. GI: 9506488 (NM_019145.1; SEQ ID NO: 275; reverse complement, SEQ ID NO: 276). The sequence of Macaca fascicularis CHRNG mRNA can be found at, for example, GenBank Accession No. GI: 982288092 (XM_005574625.3; SEQ ID NO: 277; reverse complement, SEQ ID NO: 278). The sequence of Macaca mulatta CHRNG mRNA can be found at, for example, GenBank Accession No. GI: 1622852538 (XM_028831233.1; SEQ ID NO: 279; reverse complement, SEQ ID NO: 280).

Additional examples of CHRNG mRNA sequences are readily available through publicly available databases, e.g., GenBank, UniProt, OMIM, and the Macaca genome project web site. Further information on CHRNG can be found, for example, at www.ncbi.nlm.nih.gov/gene/?term=CHRNG.

The entire contents of each of the foregoing GenBank Accession numbers and the Gene database numbers are incorporated herein by reference as of the date of filing this application.

The term CHRNG, as used herein, also refers to variations of the CHRNG gene including variants provided in the SNP database. Numerous sequence variations within the CHRNG gene have been identified and may be found at, for example, NCBI dbSNP and UniProt (see, e.g., www.ncbi.nlm.nih.gov/snp/?term=CHRNG, the entire contents of which is incorporated herein by reference as of the date of filing this application.

As used herein, “Collagen Type XIII Alpha 1 Chain,” used interchangeably with the term “COL13A1,” refers to a synaptic extracellular-matrix protein involved in the formation and maintenance of the neuromuscular synapse. COL13A1 encodes the collagen type XIII alpha1 chain (COL13A1), which is a single-pass type II transmembrane protein made of a short intracellular domain, a single transmembrane domain, and a triple-helical collagenous ectodomain. Studies have shown that patients with COL13A1 mutations underlie a myasthenic syndrome characterized by early onset muscle weakness with predominantly feeding and breathing difficulties often requiring ventilation and artificial feeding. COL13A1 is also known as COLXIIIA1, Collagen Alpha-1(XIII) Chain, or CMS19.

The sequence of a human COL13A1 mRNA transcript can be found at, for example, GenBank Accession No. GI: 1677498641 (NM_001130103.2; SEQ ID NO: 281; reverse complement, SEQ ID NO: 282). The sequence of mouse COL13A1 mRNA can be found at, for example, GenBank Accession No. GI: 755571593 (NM_007731.3; SEQ ID NO: 283; reverse complement, SEQ ID NO: 284). The sequence of rat COL13A1 mRNA can be found at, for example, GenBank Accession No. GI: 157821424 (NM_001109172.1; SEQ ID NO: 285; reverse complement, SEQ ID NO: 286). The sequence of Macaca fascicularis COL13A1 mRNA can be found at, for example, GenBank Accession No. GI: 982269148 (XM_015456252.1; SEQ ID NO: 287; reverse complement, SEQ ID NO: 288). The sequence of Macaca mulatta COL13A1 mRNA can be found at, for example, GenBank Accession No. GI: 1622966101 (XM_015147482.2; SEQ ID NO: 289; reverse complement, SEQ ID NO: 290).

Additional examples of COL13A1 mRNA sequences are readily available through publicly available databases, e.g., GenBank, UniProt, OMIM, and the Macaca genome project web site. Further information on COL13A1 can be found, for example, at www.ncbi.nlm.nih.gov/gene/?term=COL13A1.

The entire contents of each of the foregoing GenBank Accession numbers and the Gene database numbers are incorporated herein by reference as of the date of filing this application.

The term COL13A1, as used herein, also refers to variations of the COL13A1 gene including variants provided in the SNP database. Numerous sequence variations within the COL13A1 gene have been identified and may be found at, for example, NCBI dbSNP and UniProt (see, e.g., www.ncbi.nlm.nih.gov/snp/?term=COL13A1, the entire contents of which is incorporated herein by reference as of the date of filing this application.

As used herein, “Docking Protein 7,” used interchangeably with the term “DOK7,” refers to a protein that is essential for neuromuscular synaptogenesis. The protein functions in aneural activation of muscle-specific receptor kinase, which is required for postsynaptic differentiation, and in the subsequent clustering of the acetylcholine receptor in myotubes. This protein can also induce autophosphorylation of muscle-specific receptor kinase. Mutations in this gene are a cause of congenital myasthenic syndrome. DOK7 is also known as C₄orf25, Downstream Of Tyrosine Kinase 7, FLJ33718, FLJ39137, Chromosome 4 Open Reading Frame 25, CMS10, CMS1B, or FADS3.

The sequence of a human DOK7 mRNA transcript can be found at, for example, GenBank Accession No. GI: 1519242777 (NM_173660.5; SEQ ID NO: 291; reverse complement, SEQ ID NO: 292). The sequence of mouse DOK7 mRNA can be found at, for example, GenBank Accession No. GI: 1143077055 (NM_001348478.1; SEQ ID NO: 293; reverse complement, SEQ ID NO: 294). The sequence of rat DOK7 mRNA can be found at, for example, GenBank Accession No. GI: 194240570 (NM_001130062.1; SEQ ID NO: 295; reverse complement, SEQ ID NO: 296). The sequence of Macaca fascicularis DOK7 mRNA can be found at, for example, GenBank Accession No. GI: 982247946 (XM_015450057.1; SEQ ID NO: 297; reverse complement, SEQ ID NO: 298). The sequence of Macaca mulatta DOK7 mRNA can be found at, for example, GenBank Accession No. GI: 1622938489 (XM_015137905.2; SEQ ID NO: 299; reverse complement, SEQ ID NO: 300).

Additional examples of DOK7 mRNA sequences are readily available through publicly available databases, e.g., GenBank, UniProt, OMIM, and the Macaca genome project web site. Further information on DOK7 can be found, for example, at www.ncbi.nlm.nih.gov/gene/?term=DOK7.

The entire contents of each of the foregoing GenBank Accession numbers and the Gene database numbers are incorporated herein by reference as of the date of filing this application.

The term DOK7, as used herein, also refers to variations of the DOK7 gene including variants provided in the SNP database. Numerous sequence variations within the DOK7 gene have been identified and may be found at, for example, NCBI dbSNP and UniProt (see, e.g., www.ncbi.nlm.nih.gov/snp/?term=DOK7, the entire contents of which is incorporated herein by reference as of the date of filing this application.

As used herein, “LDL Receptor Related Protein 4,” used interchangeably with the term “LRP4,” refers to a member of the low-density lipoprotein receptor-related protein family. LRP4 is a single-transmembrane protein that possesses a large extracellular domain with multiple LDLR repeats, EGF-like and β-propeller repeats; a transmembrane domain; and a short C-terminal region without an identifiable catalytic motif. Mice lacking LRP4 die at birth and do not form the NMJ, indicating a critical role in neuromuscular junction (NMJ) formation. LPR4 mutation or malfunction is implicated in disorders including congenital myasthenic syndrome, myasthenia gravis, and diseases of bone or kidney. LRP4 is also known as MEGF7, LRP-4, SOST2, CLSS, Low-Density Lipoprotein Receptor-Related Protein 4, Multiple Epidermal Growth Factor-Like Domains 7, LRP10, KIAA0816, or CMS17.

The sequence of a human LRP4 mRNA transcript can be found at, for example, GenBank Accession No. GI: 1519312025 (NM_002334.4; SEQ ID NO: 301; reverse complement, SEQ ID NO: 302). The sequence of mouse LRP4 mRNA can be found at, for example, GenBank Accession No. GI: 224994222 (NM_172668.3; SEQ ID NO: 303; reverse complement, SEQ ID NO: 304). The sequence of rat LRP4 mRNA can be found at, for example, GenBank Accession No. GI: 329112575 (NM_031322.3; SEQ ID NO: 305; reverse complement, SEQ ID NO: 306). The sequence of Macaca fascicularis LRP4 mRNA can be found at, for example, GenBank Accession No. GI: 982294148 (XM_005578015.2; SEQ ID NO: 307; reverse complement, SEQ ID NO: 308). The sequence of Macaca mulatta LRP4 mRNA can be found at, for example, GenBank Accession No. GI: 1622863351 (XM_015114355.2; SEQ ID NO: 309; reverse complement, SEQ ID NO: 310).

Additional examples of LRP4 mRNA sequences are readily available through publicly available databases, e.g., GenBank, UniProt, OMIM, and the Macaca genome project web site. Further information on LRP4 can be found, for example, at www.ncbi.nlm.nih.gov/gene/?term=LRP4.

The entire contents of each of the foregoing GenBank Accession numbers and the Gene database numbers are incorporated herein by reference as of the date of filing this application.

The term LRP4, as used herein, also refers to variations of the LRP4 gene including variants provided in the SNP database. Numerous sequence variations within the LRP4 gene have been identified and may be found at, for example, NCBI dbSNP and UniProt (see, e.g., www.ncbi.nlm.nih.gov/snp/?term=LRP4, the entire contents of which is incorporated herein by reference as of the date of filing this application.

As used herein, “Muscle Associated Receptor Tyrosine Kinase,” used interchangeably with the term “MUSK,” refers to a muscle-specific tyrosine kinase receptor, which plays a central role in the formation and the maintenance of the neuromuscular junction (NMJ), the synapse between the motor neuron and the skeletal muscle. Recruitment of AGRIN by LRP4 to the MUSK signaling complex induces phosphorylation and activation of MUSK, the kinase of the complex. The activation of MUSK in myotubes regulates the formation of NMJs through the regulation of different processes including the specific expression of genes in subsynaptic nuclei, the reorganization of the actin cytoskeleton and the clustering of the acetylcholine receptors in the postsynaptic membrane. Mutations in this gene have been associated with congenital myasthenic syndrome. MUSK is also known as EC 2.7.10.1, FADS1, CMS9, FADS, Muscle, Skeletal Receptor Tyrosine-Protein Kinase, or Muscle-Specific Kinase Receptor.

The sequence of a human MUSK mRNA transcript can be found at, for example, GenBank Accession No. GI: 1609044119 (NM_005592.4; SEQ ID NO: 311; reverse complement, SEQ ID NO: 312). The sequence of mouse MUSK mRNA can be found at, for example, GenBank Accession No. GI: 260267047 (NM_001037127.2; SEQ ID NO: 313; reverse complement, SEQ ID NO: 314). The sequence of rat MUSK mRNA can be found at, for example, GenBank Accession No. GI: 1937920431 (NM_031061.2; SEQ ID NO: 315; reverse complement, SEQ ID NO: 316). The sequence of Macaca fascicularis MUSK mRNA can be found at, for example, GenBank Accession No. GI: 982300549 (XM_005581093.2; SEQ ID NO: 317; reverse complement, SEQ ID NO: 318). The sequence of Macaca mulatta MUSK mRNA can be found at, for example, GenBank Accession No. GI: 1622871800 (XM_015117113.2; SEQ ID NO: 319; reverse complement, SEQ ID NO: 320).

Additional examples of MUSK mRNA sequences are readily available through publicly available databases, e.g., GenBank, UniProt, OMIM, and the Macaca genome project web site. Further information on MUSK can be found, for example, at www.ncbi.nlm.nih.gov/gene/?term=MUSK.

The entire contents of each of the foregoing GenBank Accession numbers and the Gene database numbers are incorporated herein by reference as of the date of filing this application.

The term MUSK, as used herein, also refers to variations of the MUSK gene including variants provided in the SNP database. Numerous sequence variations within the MUSK gene have been identified and may be found at, for example, NCBI dbSNP and UniProt (see, e.g., www.ncbi.nlm.nih.gov/snp/?term=MUSK, the entire contents of which is incorporated herein by reference as of the date of filing this application.

As used herein, “Receptor Associated Protein Of The Synapse,” used interchangeably with the term “RAPSN,” refers to a member of a family of proteins that are receptor associated proteins of the synapse. The encoded protein contains a conserved cAMP-dependent protein kinase phosphorylation site, and plays a critical role in clustering and anchoring nicotinic acetylcholine receptors at synaptic sites by linking the receptors to the underlying postsynaptic cytoskeleton, possibly by direct association with actin or spectrin. Mutations in this gene may play a role in postsynaptic congenital myasthenic syndromes. RAPSN is also known as RNF205, 43 KDa Receptor-Associated Protein Of The Synapse, RING Finger Protein 205, CMS1D, CMS1E, Acetylcholine Receptor-Associated 43 Kda Protein, RAPSYN, CMS 11, CMS4C, FADS2, or FADS.

The sequence of a human RAPSN mRNA transcript can be found at, for example, GenBank Accession No. GI: 1519241818 (NM_005055.5; SEQ ID NO: 321; reverse complement, SEQ ID NO: 322). The sequence of mouse RAPSN mRNA can be found at, for example, GenBank Accession No. GI: 224967080 (NM_009023.3; SEQ ID NO: 323; reverse complement, SEQ ID NO: 324). The sequence of rat RAPSN mRNA can be found at, for example, GenBank Accession No. GI: 157819696 (NM_001108584.1; SEQ ID NO: 325; reverse complement, SEQ ID NO: 326). The sequence of Macaca fascicularis RAPSN mRNA can be found at, for example, GenBank Accession No. GI: 982294016 (XM_015434747.1; SEQ ID NO: 327; reverse complement, SEQ ID NO: 328). The sequence of Macaca mulatta RAPSN mRNA can be found at, for example, GenBank Accession No. GI: 1622863236 (XM_015114296.2; SEQ ID NO: 329; reverse complement, SEQ ID NO: 330).

Additional examples of RAPSN mRNA sequences are readily available through publicly available databases, e.g., GenBank, UniProt, OMIM, and the Macaca genome project web site. Further information on RAPSN can be found, for example, at www.ncbi.nlm.nih.gov/gene/?term=RAPSN.

The entire contents of each of the foregoing GenBank Accession numbers and the Gene database numbers are incorporated herein by reference as of the date of filing this application.

The term RAPSN, as used herein, also refers to variations of the RAPSN gene including variants provided in the SNP database. Numerous sequence variations within the RAPSN gene have been identified and may be found at, for example, NCBI dbSNP and UniProt (see, e.g., www.ncbi.nlm.nih.gov/snp/?term=RAPSN, the entire contents of which is incorporated herein by reference as of the date of filing this application.

As used herein, “Sodium Voltage-Gated Channel Alpha Subunit 4,” used interchangeably with the term “SCN4A,” refers to a member of the voltage-gated sodium channel family. Voltage-gated sodium channels are transmembrane glycoprotein complexes composed of a large alpha subunit with 24 transmembrane domains and one or more regulatory beta subunits. They are responsible for the generation and propagation of action potentials in neurons and muscle. This gene encodes one member of the sodium channel alpha subunit gene family. It is expressed in skeletal muscle, and mutations in this gene have been linked to congenital myasthenic syndrome, and several myotonia and periodic paralysis disorders. SCN4A is also known as SkM1, Nav1.4, HYPP, Sodium Channel Protein Skeletal Muscle Subunit Alpha, Voltage-Gated Sodium Channel Subunit Alpha Nav1.4, HYKPP, Skeletal Muscle Voltage-Dependent Sodium Channel Type IV Alpha Subunit, CTC-264K15.6, Na(V)1.4, HOKPP2, CMS16, or NAC1A.

The sequence of a human SCN4A mRNA transcript can be found at, for example, GenBank Accession No. GI: 93587341 (NM_000334.4; SEQ ID NO: 331; reverse complement, SEQ ID NO: 332). The sequence of mouse SCN4A mRNA can be found at, for example, GenBank Accession No. GI: 134948031 (NM_133199.2; SEQ ID NO: 333; reverse complement, SEQ ID NO: 334). The sequence of rat SCN4A mRNA can be found at, for example, GenBank Accession No. GI: 1937369400 (NM_013178.2; SEQ ID NO: 335; reverse complement, SEQ ID NO: 336). The sequence of Macaca fascicularis SCN4A mRNA can be found at, for example, GenBank Accession No. GI: 982306407 (XM_015438708.1; SEQ ID NO: 337; reverse complement, SEQ ID NO: 338). The sequence of Macaca mulatta SCN4A mRNA can be found at, for example, GenBank Accession No. GI: 1622880585 (XM_015120096.2; SEQ ID NO: 339; reverse complement, SEQ ID NO: 340).

Additional examples of SCN4A mRNA sequences are readily available through publicly available databases, e.g., GenBank, UniProt, OMIM, and the Macaca genome project web site. Further information on SCN4A can be found, for example, at www.ncbi.nlm.nih.gov/gene/?term=SCN4A.

The entire contents of each of the foregoing GenBank Accession numbers and the Gene database numbers are incorporated herein by reference as of the date of filing this application.

The term SCN4A, as used herein, also refers to variations of the SCN4A gene including variants provided in the SNP database. Numerous sequence variations within the SCN4A gene have been identified and may be found at, for example, NCBI dbSNP and UniProt (see, e.g., www.ncbi.nlm.nih.gov/snp/?term=SCN4A, the entire contents of which is incorporated herein by reference as of the date of filing this application.

As used herein, “Double Homeobox 4,” used interchangeably with the term “DUX4,” refers to a transcriptional activator of many genes. DUX4 is normally expressed during early embryonic development, and is then effectively silenced in all tissues except the testis and thymus. DUX4 has been implicated as being involved in cell death, oxidative stress, muscle differentiation and growth, epigenetic regulation, and a number of other signaling pathways in skeletal muscle. Inappropriate expression of DUX4 in muscle cells is the cause of facioscapulohumeral muscular dystrophy (FSHD), which is characterized by muscle weakness and wasting (atrophy) that worsens slowly over time. DUX4 is also known as Double Homeobox Protein 10, Double Homeobox Protein 4, Double Homeobox Protein 4/10, DUX4L, and DUX10.

The sequence of a human DUX4 mRNA transcript can be found at, for example, GenBank Accession No. GI: 1774753171 (NM_001306068.3; SEQ ID NO: 341; reverse complement, SEQ ID NO: 342). The sequence of mouse DUX4 mRNA can be found at, for example, GenBank Accession No. GI: 126432555 (NM_001081954.1; SEQ ID NO: 343; reverse complement, SEQ ID NO: 344). The sequence of rat DUX4 mRNA can be found at, for example, GenBank Accession No. GI: 1958689769 (XM_008771031.3; SEQ ID NO: 345; reverse complement, SEQ ID NO: 346). The sequence of Macaca mulatta DUX4 mRNA can be found at, for example, GenBank Accession No. GI: 1622942424 (XM_028848991.1; SEQ ID NO: 347; reverse complement, SEQ ID NO: 348).

Additional examples of DUX4 mRNA sequences are readily available through publicly available databases, e.g., GenBank, UniProt, OMIM, and the Macaca genome project web site. Further information on DUX4 can be found, for example, at www.ncbi.nlm.nih.gov/gene/?term=DUX4.

The entire contents of each of the foregoing GenBank Accession numbers and the Gene database numbers are incorporated herein by reference as of the date of filing this application.

The term DUX4, as used herein, also refers to variations of the DUX4 gene including variants provided in the SNP database. Numerous sequence variations within the DUX4 gene have been identified and may be found at, for example, NCBI dbSNP and UniProt (see, e.g., www.ncbi.nlm.nih.gov/snp/?term=DUX4, the entire contents of which is incorporated herein by reference as of the date of filing this application.

As used herein, “phospholamban,” used interchangeably with the term “PLN,” refers to a crucial regulator of cardiac contractility. PLN is a major substrate for the cAMP-dependent protein kinase in cardiac muscle. The encoded protein is an inhibitor of cardiac muscle sarcoplasmic reticulum Ca(2+)-ATPase in the unphosphorylated state, but inhibition is relieved upon phosphorylation of the protein. The subsequent activation of the Ca(2+) pump leads to enhanced muscle relaxation rates, thereby contributing to the inotropic response elicited in heart by beta-agonists. The encoded protein is a key regulator of cardiac diastolic function. Mutations in this gene are a cause of inherited human dilated cardiomyopathy with refractory congestive heart failure, and also familial hypertrophic cardiomyopathy. PLN is also known as CMD1P, PLB, Cardiac Phospholamban, or CMH.

An exemplary sequence of a human PLN mRNA transcript can be found at, for example, GenBank Accession No. GI: 1519242997 (NM_002667.5; SEQ ID NO: 349; reverse complement, SEQ ID NO: 350). The sequence of mouse PLN mRNA can be found at, for example, GenBank Accession No. GI: 213512815 (NM_001141927.1; SEQ ID NO: 351; reverse complement, SEQ ID NO: 352). The sequence of rat PLN mRNA can be found at, for example, GenBank Accession No. GI: 399124783 (NM_022707.2; SEQ ID NO: 353; reverse complement, SEQ ID NO: 354). The sequence of Macaca mulatta PLN mRNA can be found at, for example, GenBank Accession No. GI: 1863319929 (NM_001190894.2; SEQ ID NO: 355; reverse complement, SEQ ID NO: 356).

Additional examples of PLN mRNA sequences are readily available through publicly available databases, e.g., GenBank, UniProt, OMIM, UCSC Genome Browser, and the Macaca genome project web site. Further information on PLN can be found, for example, at www.ncbi.nlm.nih.gov/gene/?term=PLN.

The entire contents of each of the foregoing GenBank Accession numbers and the Gene database numbers are incorporated herein by reference as of the date of filing this application.

The term PLN, as used herein, also refers to variations of the PLN gene including variants provided in the SNP database. Numerous sequence variations within the PLN gene have been identified and may be found at, for example, NCBI dbSNP and UniProt (see, e.g., www.ncbi.nlm.nih.gov/snp/?term=PLN, the entire contents of which is incorporated herein by reference as of the date of filing this application.

As used herein, “calcium/calmodulin dependent protein kinase II delta,” used interchangeably with the term “CAMK2D,” refers to a member of the serine/threonine protein kinase family and the Ca(2+)/calmodulin-dependent protein kinase subfamily. CAMK2D is involved in the regulation of Ca(2+) homeostatis and excitation-contraction coupling in heart by targeting ion channels, transporters and accessory proteins involved in Ca(2+) influx into the myocyte, Ca(2+) release from the sarcoplasmic reticulum (SR), SR Ca(2+) uptake and Na(+) and K(+) channel transport. CAMK2D also targets transcription factors and signaling molecules to regulate heart function. In its activated form, CAMK2D is involved in the pathogenesis of dilated cardiomyopathy and heart failure. CAMK2D contributes to cardiac decompensation and heart failure by regulating SR Ca(2+) release via direct phosphorylation of RYR2 Ca(2+) channel. In the nucleus, CAMK2D phosphorylates the MEF2 repressor HDAC4, promoting its nuclear export and binding to 14-3-3 protein, and expression of MEF2 and genes involved in the hypertrophic program. CAMK2D is essential for left ventricular remodeling responses to myocardial infarction. In pathological myocardial remodeling, CAMK2D acts downstream of the beta adrenergic receptor signaling cascade to regulate key proteins involved in excitation-contraction coupling. CAMK2D regulates Ca(2+) influx to myocytes by binding and phosphorylating the L-type Ca(2+) channel subunit beta-2 CACNB2. In addition to Ca(2+) channels, CAMK2D can target and regulate the cardiac sarcolemmal Na(+) channel Nav1.5/SCN5A and the K+ channel Kv4.3/KCND3, which contribute to arrhythmogenesis in heart failure. CAMK2D phosphorylates phospholamban (PLN), an endogenous inhibitor of SERCA2A/ATP2A2, contributing to the enhancement of SR Ca(2+) uptake that may be important in frequency-dependent acceleration of relaxation and maintenance of contractile function during acidosis. CAMK2D may participate in the modulation of skeletal muscle function in response to exercise, by regulating SR Ca(2+) transport through phosphorylation of PLN and triadin, a ryanodine receptor-coupling factor. CAMK2D is also known as Calcium/Calmodulin-Dependent Protein Kinase Type II Delta Chain, CaM Kinase II Delta Subunit, CaM Kinase II Subunit Delta, CAMKD, EC 2.7.11.17, or EC 2.7.11.

An exemplary sequence of a human CAMK2D mRNA transcript can be found at, for example, GenBank Accession No. GI: 1519243899 (NM_001321571.2; SEQ ID NO: 357; reverse complement, SEQ ID NO: 358). The sequence of mouse CAMK2D mRNA can be found at, for example, GenBank Accession No. GI: 654824235 (NM_001025439.2; SEQ ID NO: 359; reverse complement, SEQ ID NO: 360). The sequence of rat CAMK2D mRNA can be found at, for example, GenBank Accession No. GI: 144922682 (NM_012519.2; SEQ ID NO: 361; reverse complement, SEQ ID NO: 362). The sequence of Macaca mulatta CAMK2D mRNA can be found at, for example, GenBank Accession No. GI: 1622941163 (XM_015139100.2; SEQ ID NO: 363; reverse complement, SEQ ID NO: 364).

Additional examples of CAMK2D mRNA sequences are readily available through publicly available databases, e.g., GenBank, UniProt, OMIM, UCSC Genome Browser, and the Macaca genome project web site. Further information on CAMK2D can be found, for example, at www.ncbi.nlm.nih.gov/gene/?term=CAMK2D.

The entire contents of each of the foregoing GenBank Accession numbers and the Gene database numbers are incorporated herein by reference as of the date of filing this application.

The term CAMK2D, as used herein, also refers to variations of the CAMK2D gene including variants provided in the SNP database. Numerous sequence variations within the CAMK2D gene have been identified and may be found at, for example, NCBI dbSNP and UniProt (see, e.g., www.ncbi.nlm.nih.gov/snp/?term=CAMK2D, the entire contents of which is incorporated herein by reference as of the date of filing this application.

As used herein, “dystrophy myotonic protein kinase,” used interchangeably with the term “DMPK,” refers to a non-receptor serine/threonine protein kinase which is necessary for the maintenance of skeletal muscle structure and function. DMPK plays a role in myocyte differentiation and survival by regulating the integrity of the nuclear envelope and the expression of muscle-specific genes. DMPK also phosphorylates PPP1R12A and inhibits the myosin phosphatase activity to regulate myosin phosphorylation. DMPK is also critical to the modulation of cardiac contractility and to the maintenance of proper cardiac conduction activity probably through the regulation of cellular calcium homeostasis. DMPK phosphorylates PLN, a regulator of calcium pumps and may regulate sarcoplasmic reticulum calcium uptake in myocytes. DMPK also phosphorylates FXYD1/PLM which is able to induce chloride currents, and may play a role in synaptic plasticity. DMPK is also known as DM1 protein kinase, DM1PK, DM1, MT-PK, MDPK, DMK, myotonin-protein kinase, myotonic dystrophy associated protein kinase, dystrophia myotonica protein kinase, myotonin protein kinase A, thymopoietin homolog, or EC 2.7.11.1.

An exemplary sequence of a human DMPK mRNA transcript can be found at, for example, GenBank Accession No. GI: 571026697 (NM_001081563.2; SEQ ID NO: 365; reverse complement, SEQ ID NO: 366). The sequence of mouse DMPK mRNA can be found at, for example, GenBank Accession No. GI: 1824718155 (NM_032418.3; SEQ ID NO: 367; reverse complement, SEQ ID NO: 368). The sequence of rat DMPK mRNA can be found at, for example, GenBank Accession No. GI: 1719749725 (NM_001372064.1; SEQ ID NO: 369; reverse complement, SEQ ID NO: 370). The sequence of Macaca fascicularis DMPK mRNA can be found at, for example, GenBank Accession No. GI: 2161880869 (XM_045381179.1; SEQ ID NO: 371; reverse complement, SEQ ID NO: 372).

Additional examples of DMPK mRNA sequences are readily available through publicly available databases, e.g., GenBank, UniProt, OMIM, UCSC Genome Browser, and the Macaca genome project web site. Further information on DMPK can be found, for example, at www.ncbi.nlm.nih.gov/gene/?term=DMPK.

The entire contents of each of the foregoing GenBank Accession numbers and the Gene database numbers are incorporated herein by reference as of the date of filing this application.

The term DMPK, as used herein, also refers to variations of the DMPK gene including variants provided in the SNP database. Numerous sequence variations within the DMPK gene have been identified and may be found at, for example, NCBI dbSNP and UniProt (see, e.g., www.ncbi.nlm.nih.gov/snp/?term=DMPK, the entire contents of which is incorporated herein by reference as of the date of filing this application.

As used herein, “glycogen synthase 1,” used interchangeably with the term “GYS1,” refers to an enzyme that catalyzes the addition of glucose monomers to the growing glycogen molecule. Glycogen is a major source of stored energy in the body. Most glucose that is taken in from food is stored as glycogen in muscle cells. During contractions of the cardiac muscle or rapid or sustained movement of skeletal muscle, glycogen stored in muscle cells is broken down to supply the cells with energy. GYS1 is produced in most cells but is most abundant in heart (cardiac) muscle and muscles used for movement (skeletal muscles). Mutations in the GYS1 gene have been found to cause a form of glycogen storage disease type 0 (GSD 0) that affects cardiac and skeletal muscle. Most GYS1 gene mutations that cause this condition lead to a lack of functional muscle glycogen synthase, resulting in a complete absence of glycogen in muscle cells. Normally, glycogen is formed from the leftover glucose that is not immediately used by cells after glucose is consumed during meals. In people with GSD 0, who cannot form glycogen, the extra sugar is released by the body. As a result, people with muscle GSD 0 do not have any stored energy, which leads to muscle pain, weakness, or episodes of fainting following moderate physical activity. Since there is no glycogen in cardiac muscle, affected individuals are also at an increased risk of cardiac arrest and sudden death, particularly after physical activity. GYS1 is also known as muscle glycogen synthase, GSY, GYS, or EC 2.4.1.11.

An exemplary sequence of a human GYS1 mRNA transcript can be found at, for example, GenBank Accession No. GI: 1519246122 (NM_002103.5; SEQ ID NO: 373; reverse complement, SEQ ID NO: 374). The sequence of mouse GYS1 mRNA can be found at, for example, GenBank Accession No. GI: 119672917 (NM_030678.3; SEQ ID NO: 375; reverse complement, SEQ ID NO: 376). The sequence of rat GYS1 mRNA can be found at, for example, GenBank Accession No. GI: 157823921 (NM_001109615.1; SEQ ID NO: 377; reverse complement, SEQ ID NO: 378). The sequence of Macaca fascicularis GYS1 mRNA can be found at, for example, GenBank Accession No. GI: 2161874347 (XM_005589837.3; SEQ ID NO: 379; reverse complement, SEQ ID NO: 380).

Additional examples of GYS1 mRNA sequences are readily available through publicly available databases, e.g., GenBank, UniProt, OMIM, UCSC Genome Browser, and the Macaca genome project web site. Further information on GYS1 can be found, for example, at www.ncbi.nlm.nih.gov/gene/?term=GYS1.

The entire contents of each of the foregoing GenBank Accession numbers and the Gene database numbers are incorporated herein by reference as of the date of filing this application.

The term GYS1, as used herein, also refers to variations of the GYS1 gene including variants provided in the SNP database. Numerous sequence variations within the GSY gene have been identified and may be found at, for example, NCBI dbSNP and UniProt (see, e.g., www.ncbi.nlm.nih.gov/snp/?term=GYS1, the entire contents of which is incorporated herein by reference as of the date of filing this application.

As used herein, “survival of motor neuron 1,” used interchangeably with the term “SMN1,” refers to one of a group of proteins called the SMN complex, which is important for the maintenance of specialized nerve cells called motor neurons. These cells are located in the spinal cord and the part of the brain that is connected to the spinal cord (the brainstem). Motor neurons transmit signals from the brain and spinal cord that tell skeletal muscles to tense (contract), which allows the body to move. In cells, the SMN complex plays an important role in processing mRNA. The SMN complex helps to assemble the cellular machinery needed to process pre-mRNA. The SMN complex is also important for the development of specialized outgrowths from nerve cells called dendrites and axons. Dendrites and axons are required for the transmission of impulses between neurons and from neurons to muscles. Many mutations in the SMN1 gene have been found to cause spinal muscular atrophy. This condition is characterized by a loss of motor neurons that leads to weakness and wasting (atrophy) in muscles used for movement (skeletal muscles) that worsens with age. SMN1 is also known as SMNT, TDRD16A, Gemin-1, BCD541, GEMINI, SMA1, SMA2, SMA3, SMA4, SMN, SMNT, tudoe domain containing 16A, complement of gems 1, or SMNC.

An exemplary sequence of a human SMN1 mRNA transcript can be found at, for example, GenBank Accession No. GI: 663070993 (NM_001297715.1; SEQ ID NO: 381; reverse complement, SEQ ID NO: 382). The sequence of mouse SMN1 mRNA can be found at, for example, GenBank Accession No. GI: 145386573 (NM_011420.2; SEQ ID NO: 383; reverse complement, SEQ ID NO: 384). The sequence of rat SMN1 mRNA can be found at, for example, GenBank Accession No. GI: 1939402010 (NM_022509.2; SEQ ID NO: 385; reverse complement, SEQ ID NO: 386). The sequence of Macaca mulatta SMN1 mRNA can be found at, for example, GenBank Accession No. GI: 386781228 (NM_001260664.1; SEQ ID NO: 387; reverse complement, SEQ ID NO: 388).

Additional examples of SMN1 mRNA sequences are readily available through publicly available databases, e.g., GenBank, UniProt, OMIM, UCSC Genome Browser, and the Macaca genome project web site. Further information on SMN1 can be found, for example, at www.ncbi.nlm.nih.gov/gene/?term=SMN1.

The entire contents of each of the foregoing GenBank Accession numbers and the Gene database numbers are incorporated herein by reference as of the date of filing this application.

The term SMN1, as used herein, also refers to variations of the SMN1 gene including variants provided in the SNP database. Numerous sequence variations within the SMN1 gene have been identified and may be found at, for example, NCBI dbSNP and UniProt (see, e.g., www.ncbi.nlm.nih.gov/snp/?term=SMN1, the entire contents of which is incorporated herein by reference as of the date of filing this application.

As used herein, “alpha-glucosidase,” used interchangeably with the term “GAA,” refers to an enzyme responsible for the degradation of glycogen to glucose in lysosomes. More than 200 mutations in the GAA gene have been identified in people with Pompe disease. Many of these mutations change one of the protein building blocks (amino acids) used to make acid alpha-glucosidase. Other mutations insert or delete genetic material in the GAA gene. Mutations in this gene significantly reduce the activity of acid alpha-glucosidase, preventing the enzyme from breaking down glycogen effectively. As a result, this complex sugar can build up to toxic levels in lysosomes.

The abnormal buildup of glycogen damages organs and tissues throughout the body, particularly the muscles, leading to progressive muscle weakness, heart problems, and the other features of Pompe disease. GAA is also known as lysosomal alpha-glucosidase, acid maltase, EC 3.2.1.20, glycogen storage disease type II, or LYAG.

An exemplary sequence of a human GAA mRNA transcript can be found at, for example, GenBank Accession No. GI: 1519245858 (NM_000152.5; SEQ ID NO: 389; reverse complement, SEQ ID NO: 390). The sequence of mouse GAA mRNA can be found at, for example, GenBank Accession No. GI: 957579368 (NM_008064.4; SEQ ID NO: 391; reverse complement, SEQ ID NO: 392). The sequence of rat GAA mRNA can be found at, for example, GenBank Accession No. GI: 40018605 (NM_199118.1; SEQ ID NO: 393; reverse complement, SEQ ID NO: 394). The sequence of Macaca mulatta GAA mRNA can be found at, for example, GenBank Accession No. GI: 1622881859 (XM_015120499.2; SEQ ID NO: 395; reverse complement, SEQ ID NO: 396).

Additional examples of GAA mRNA sequences are readily available through publicly available databases, e.g., GenBank, UniProt, OMIM, UCSC Genome Browser, and the Macaca genome project web site. Further information on GAA can be found, for example, at www.ncbi.nlm.nih.gov/gene/?term=GAA.

The entire contents of each of the foregoing GenBank Accession numbers and the Gene database numbers are incorporated herein by reference as of the date of filing this application.

The term GAA, as used herein, also refers to variations of the GAA gene including variants provided in the SNP database. Numerous sequence variations within the GAA gene have been identified and may be found at, for example, NCBI dbSNP and UniProt (see, e.g., www.ncbi.nlm.nih.gov/snp/?term=GAA, the entire contents of which is incorporated herein by reference as of the date of filing this application.

As used herein, “Mucin 5B, Oligomeric Mucus/Gel-Forming,” used interchangeably with the term “MUC5B,” refers to a member of the mucin family of proteins, which are highly glycosylated macromolecular components of mucus secretions. MUB5B is the major gel-forming mucin in mucus. It is a major contributor to the lubricating and viscoelastic properties of whole saliva, normal lung mucus and cervical mucus. This gene has been found to be up-regulated in some human diseases, including sinus mucosa of chronic rhinosinusitis (CRS), CRS with nasal polyposis, chronic obstructive pulmonary disease (COPD) and H. pylori-associated gastric disease. MUC5B is also known as mucin-5B, MUC5, MG1, high molecular weight salivary mucin MG1; mucin 5, subtype B, tracheobronchial; sublingual gland mucin; cervical mucin, or MUC9.

An exemplary sequence of a human MUC5B mRNA transcript can be found at, for example, GenBank Accession No. GI: 1519244536 (NM_002458.3; SEQ ID NO:397; reverse complement, SEQ ID NO: 398). The sequence of mouse MUC5B mRNA can be found at, for example, GenBank Accession No. GI: 147905739 (NM_028801.2; SEQ ID NO:399; reverse complement, SEQ ID NO: 400). The sequence of rat MUC5B mRNA can be found at, for example, GenBank Accession No. GI: 1958654562 (XM_039101271.1; SEQ ID NO:401; reverse complement, SEQ ID NO: 402). The sequence of Macaca mulatta MUC5B mRNA can be found at, for example, GenBank Accession No. GI: 1622861542 (XM_028833012.1; SEQ ID NO: 403; reverse complement, SEQ ID NO: 404).

Additional examples of MUC5B mRNA sequences are readily available through publicly available databases, e.g., GenBank, UniProt, OMIM, UCSC Genome Browser, and the Macaca genome project web site.

Further information on MUC5B can be found, for example, at www.ncbi.nlm.nih.gov/gene/?term=MUC5B.

The entire contents of each of the foregoing GenBank Accession numbers and the Gene database numbers are incorporated herein by reference as of the date of filing this application.

The term MUC5B, as used herein, also refers to variations of the MUC5B gene including variants provided in the SNP database. Numerous sequence variations within the MUC5B gene have been identified and may be found at, for example, NCBI dbSNP and UniProt (see, e.g., www.ncbi.nlm.nih.gov/gene/?term=MUC5B, the entire contents of which is incorporated herein by reference as of the date of filing this application.

As used herein, “Thymic Stromal Lymphopoietin,” used interchangeably with the term “TSLP,” refers to a hemopoietic cytokine which signals through a heterodimeric receptor complex composed of the TSLP receptor and the IL-7R alpha chain. TSLP mainly impacts myeloid cells and induces the release of T cell-attracting chemokines from monocytes and enhances the maturation of CD11c(+) dendritic cells. TSLP promotes T helper type 2 (TH2) cell responses that are associated with immunity in various inflammatory diseases, including asthma, allergic inflammation and chronic obstructive pulmonary disease.

An exemplary sequence of a human TSLP mRNA transcript can be found at, for example, GenBank Accession No. GI: 1519241510 (NM_033035.5; SEQ ID NO:405; reverse complement, SEQ ID NO: 406). The sequence of mouse TSLP mRNA can be found at, for example, GenBank Accession No. GI: 283945612 (NM_021367.2; SEQ ID NO:407; reverse complement, SEQ ID NO: 408). The sequence of rat TSLP mRNA can be found at, for example, GenBank Accession No. GI: 1958745494 (XM_039097381.1; SEQ ID NO:409; reverse complement, SEQ ID NO: 410). The sequence of Macaca mulatta TSLP mRNA can be found at, for example, GenBank Accession No. GI: 1622946249 (XM_001100503.4; SEQ ID NO: 411; reverse complement, SEQ ID NO: 412).

Additional examples of TSLP mRNA sequences are readily available through publicly available databases, e.g., GenBank, UniProt, OMIM, UCSC Genome Browser, and the Macaca genome project web site.

Further information on TSLP can be found, for example, at www.ncbi.nlm.nih.gov/gene/?term=TSLP.

The entire contents of each of the foregoing GenBank Accession numbers and the Gene database numbers are incorporated herein by reference as of the date of filing this application.

The term TSLP, as used herein, also refers to variations of the TSLP gene including variants provided in the SNP database. Numerous sequence variations within the TSLP gene have been identified and may be found at, for example, NCBI dbSNP and UniProt (see, e.g., www.ncbi.nlm.nih.gov/gene/?term=TSLP, the entire contents of which is incorporated herein by reference as of the date of filing this application.

As used herein, “Interleukin 33,” used interchangeably with the term “IL33,” refers to an alarmin cytokine from the Il-1 family. IL33 binds to and signals through the IL1RL1/ST2 receptor which in turn activates NF-kappa-B and MAPK signaling pathways in target cells. IL33 is involved in the maturation of Th2 cells inducing the secretion of T-helper type 2-associated cytokines. IL33 is also involved in activation of mast cells, basophils, eosinophils and natural killer cells. IL33 acts as a chemoattractant for Th2 cells, and function as an alarmin that amplifies immune responses during tissue injury. IL33 is also known as NF-HEV, IL1F11, C₉orf26, DVS27, Nuclear Factor From High Endothelial Venules, Interleukin-1 Family Member 11, Chromosome 9 Open Reading Frame 26 (NF-HEV), or DKFZp586H0523.

An exemplary sequence of a human IL33 mRNA transcript can be found at, for example, GenBank Accession No. GI: 1677537223 (NM_033439.4; SEQ ID NO:413; reverse complement, SEQ ID NO: 414). The sequence of mouse IL33 mRNA can be found at, for example, GenBank Accession No. GI: 1341395582 (NM_001164724.2; SEQ ID NO:415; reverse complement, SEQ ID NO: 416). The sequence of rat IL33 mRNA can be found at, for example, GenBank Accession No. GI: 62079056 (NM_001014166.1; SEQ ID NO:417; reverse complement, SEQ ID NO: 418). The sequence of Macaca mulatta IL33 mRNA can be found at, for example, GenBank Accession No. GI: 1622872669 (XM_015117709.2; SEQ ID NO: 419; reverse complement, SEQ ID NO: 420).

Additional examples of IL33 mRNA sequences are readily available through publicly available databases, e.g., GenBank, UniProt, OMIM, UCSC Genome Browser, and the Macaca genome project web site.

Further information on IL33 can be found, for example, at www.ncbi.nlm.nih.gov/gene/?term=IL33.

The entire contents of each of the foregoing GenBank Accession numbers and the Gene database numbers are incorporated herein by reference as of the date of filing this application.

The term IL33, as used herein, also refers to variations of the IL33 gene including variants provided in the SNP database. Numerous sequence variations within the IL33 gene have been identified and may be found at, for example, NCBI dbSNP and UniProt (see, e.g., www.ncbi.nlm.nih.gov/gene/?term=IL33, the entire contents of which is incorporated herein by reference as of the date of filing this application.

As used herein, “Arachidonate 15-Lipoxygenase,” used interchangeably with the term “ALOX15,” refers to a member of the lipoxygenase family of proteins. ALOX15 acts on various polyunsaturated fatty acid substrates to generate various bioactive lipid mediators such as eicosanoids, hepoxilins, lipoxins, and other molecules. The encoded enzyme and its reaction products have been shown to regulate inflammation and immunity. ALOX15 plays an important role during the maintenance of self-tolerance by peroxidizing membrane-bound phosphatidylethanolamine which can then signal the sorting process for clearance of apoptotic cells during inflammation and prevent an autoimmune response. In addition to its role in the immune and inflammatory responses, ALOX15 may play a role in epithelial wound healing in the cornea through production of lipoxin A4 (LXA(4)) and docosahexaenoic acid-derived neuroprotectin D1, both lipid autacoids exhibit anti-inflammatory and neuroprotective properties. Furthermore, ALOX15 may regulate actin polymerization which is crucial for several biological processes such as the phagocytosis of apoptotic cells. ALOX15 is also implicated in the generation of endogenous ligands for peroxisome proliferator activated receptor (PPAR-gamma), hence modulating macrophage development and function. ALOX15 may also exert a negative effect on skeletal development by regulating bone mass through this pathway. Finally, ALOX15 is also involved in the cellular response to IL13. ALOX15 is also known as 15-LOX-1, Polyunsaturated Fatty Acid Lipoxygenase ALOX15, Arachidonate 12-Lipoxygenase, Leukocyte-Type, Arachidonate Omega-6 Lipoxygenase, Hepoxilin A3 Synthase Alox15, Linoleate 13S-Lipoxygenase, 12/15-Lipoxygenase, 12-LOX, LOG15, 15-Lipoxygenase Type 1, EC 1.13.11.31, EC 1.13.11.33, EC 1.13.11.12, EC 1.13.11, or EC 1.13.11.

An exemplary sequence of a human ALOX15 mRNA transcript can be found at, for example, GenBank Accession No. GI: 1698254589 (NM_001140.5; SEQ ID NO:421; reverse complement, SEQ ID NO: 422). The sequence of mouse ALOX15 mRNA can be found at, for example, GenBank Accession No. GI: 134948632 (NM_009660.3; SEQ ID NO:423; reverse complement, SEQ ID NO: 424). The sequence of rat ALOX15 mRNA can be found at, for example, GenBank Accession No. GI: 31542124 (NM_031010.2; SEQ ID NO:425; reverse complement, SEQ ID NO: 426). The sequence of Macaca mulatta ALOX15 mRNA can be found at, for example, GenBank Accession No. GI: 1622876822 (XM_028835918.1; SEQ ID NO: 427; reverse complement, SEQ ID NO: 428).

Additional examples of ALOX15 mRNA sequences are readily available through publicly available databases, e.g., GenBank, UniProt, OMIM, UCSC Genome Browser, and the Macaca genome project web site.

Further information on ALOX15 can be found, for example, at www.ncbi.nlm.nih.gov/gene/?term=ALOX15.

The entire contents of each of the foregoing GenBank Accession numbers and the Gene database numbers are incorporated herein by reference as of the date of filing this application.

The term ALOX15, as used herein, also refers to variations of the ALOX15 gene including variants provided in the SNP database. Numerous sequence variations within the ALOX15 gene have been identified and may be found at, for example, NCBI dbSNP and UniProt (see, e.g., www.ncbi.nlm.nih.gov/gene/?term=ALOX15, the entire contents of which is incorporated herein by reference as of the date of filing this application.

As used herein, “advanced glycosylation end-product specific receptor,” used interchangeably with the terms “AGER” and “RAGE,” refers to a member of the immunoglobulin superfamily of cell surface receptors. It is a multiligand receptor, and besides AGE, interacts with other molecules implicated in homeostasis, development, and inflammation, and certain diseases, such as diabetes and Alzheimer's disease.

An exemplary sequence of a human AGER mRNA transcript can be found at, for example, GenBank Accession No. NM_001136.5 (SEQ ID NO:429; reverse complement, SEQ ID NO: 430). The sequence of mouse AGER mRNA can be found at, for example, GenBank Accession No. NM_007425.3 (SEQ ID NO:431; reverse complement, SEQ ID NO: 432). The sequence of rat AGER mRNA can be found at, for example, GenBank Accession No. NM_053336.2 (SEQ ID NO:433; reverse complement, SEQ ID NO: 434). The sequence of Macaca mulatta AGER mRNA can be found at, for example, GenBank Accession No. NM_001205117.1 (SEQ ID NO: 435; reverse complement, SEQ ID NO: 436).

Additional examples of AGER mRNA sequences are readily available through publicly available databases, e.g., GenBank (e.g., NM_001136.5; NM_001206929.2; NM_001206932.2; NM_001206934.2; NM_001206936.; NM_001206940.2; NM_001206954.2; NM_001206966.2; NM_172197.3), UniProt, OMIM, UCSC Genome Browser, and the Macaca genome project web site.

Further information on AGER can be found, for example, at www.ncbi.nlm.nih.gov/gene/?term=AGER.

The entire contents of each of the foregoing GenBank Accession numbers and the Gene database numbers are incorporated herein by reference as of the date of filing this application.

The term AGER, as used herein, also refers to variations of the AGER gene including variants provided in the SNP database. Numerous sequence variations within the AGER gene have been identified and may be found at, for example, NCBI dbSNP and UniProt (see, e.g., www.ncbi.nlm.nih.gov/snp/?term=AGER, the entire contents of which is incorporated herein by reference as of the date of filing this application.

As used herein, “mucin 5AC, oligomeric mucus/gel-forming,” used interchangeably with the terms “MUC5AC” and “MUC5,” and “TBM,” refers to a member of the extracellular matrix structural constituent involved in phosphatidylinositol-mediated signaling. Located in cytoplasm; extracellular space; and mucus layer. Biomarker of several diseases, including Sjogren's syndrome; biliary tract disease (multiple); cystic fibrosis; eye disease (multiple); and pancreatic cancer (multiple). MUC5AC is known to contribute to severe muco-obstructive lung diseases, worsening chronic obstructive pulmonary disease (COPD) pathogenesis. Genomewide association studies have highlighted the causative role of increased MUC5AC expression in the pathogenesis of moderate and severe asthma. Overexpression of airway mucins, such as MUC5AC, has been described in idiopathic pulmonary fibrosis (IPF) lungs.

An exemplary sequence of a human MUC5AC mRNA transcript can be found at, for example, GenBank Accession No. NM_001304359.2 (SEQ ID NO:437; reverse complement, SEQ ID NO: 438). The sequence of mouse MUC5AC mRNA can be found at, for example, GenBank Accession No. NM_010844.3 (SEQ ID NO:439 reverse complement, SEQ ID NO: 440). The sequence of rat MUC5AC mRNA can be found at, for example, GenBank Accession No. NM_001419868 (SEQ ID NO:441; reverse complement, SEQ ID NO: 442). The sequence of Macaca mulatta MUC5AC mRNA can be found at, for example, GenBank Accession No. XM_028832999.1 (SEQ ID NO: 443; reverse complement, SEQ ID NO: 444).

Additional examples of MUC5AC mRNA sequences are readily available through publicly available databases, e.g., GenBank, UniProt, OMIM, UCSC Genome Browser, and the Macaca genome project web site.

Further information on MUC5AC can be found, for example, at www.ncbi.nlm.nih.gov/gene/?term=MUC5AC.

The entire contents of each of the foregoing GenBank Accession numbers and the Gene database numbers are incorporated herein by reference as of the date of filing this application.

The term MUC5AC, as used herein, also refers to variations of the MUC5AC gene including variants provided in the SNP database. Numerous sequence variations within the MUC5AC gene have been identified and may be found at, for example, NCBI dbSNP and UniProt (see, e.g., www.ncbi.nlm.nih.gov/snp/?term=MUC5AC, the entire contents of which is incorporated herein by reference as of the date of filing this application.

As used herein, “signal transducer and activator of transcription factor 6,” used interchangeably with the term “STAT6,” refers to a member of the STAT family of transcription factors. In response to cytokines and growth factors, STAT family members are phosphorylated by the receptor associated kinases, and then form homo- or heterodimers that translocate to the cell nucleus where they act as transcription activators. STAT6 has been demonstrated to regulate many pathologic features of lung inflammatory responses in animal models including airway eosinophilia, epithelial mucus production, smooth muscle changes, Th2 cell differentiation, and IgE production from B cells (Wurster A L, et al., Oncogene 2000; 19:2577-84). STAT6 is also known as interleukin-4 induced, IL-4-STAT, D12S1644, STAT6B, or STAT6C.

The sequence of a human STAT6 mRNA transcript can be found at, for example, GenBank Accession No. GI: 1519313969 (NM_003153.5; SEQ ID NO:445; reverse complement, SEQ ID NO: 446). The sequence of mouse STAT6 mRNA can be found at, for example, GenBank Accession No. GI: 128485773 (NM_009284.2; SEQ ID NO:447; reverse complement, SEQ ID NO: 448). The sequence of rat STAT6 mRNA can be found at, for example, GenBank Accession No. GI: 113205499 (NM_001044250.1; SEQ ID NO:449; reverse complement, SEQ ID NO: 450). The sequence of Macaca fascicularis STAT6 mRNA can be found at, for example, GenBank Accession No. GI: 982282006 (XM_005571286.2; SEQ ID NO: 451; reverse complement, SEQ ID NO: 452). The sequence of Macaca mulatta STAT6 mRNA can be found at, for example, GenBank Accession No. GI: 1622842915 (XM_015152044.2; SEQ ID NO: 453; reverse complement, SEQ ID NO: 454).

Additional examples of STAT6 mRNA sequences are readily available through publicly available databases, e.g., GenBank, UniProt, OMIM, and the Macaca genome project web site. Further information on STAT6 can be found, for example, at www.ncbi.nlm.nih.gov/gene/?term=STAT6.

The entire contents of each of the foregoing GenBank Accession numbers and the Gene database numbers are incorporated herein by reference as of the date of filing this application.

The term STAT6, as used herein, also refers to variations of the STAT6 gene including variants provided in the SNP database. Numerous sequence variations within the STAT6 gene have been identified and may be found at, for example, NCBI dbSNP and UniProt (see, e.g., www.ncbi.nlm.nih.gov/snp/?term=STAT6, the entire contents of which is incorporated herein by reference as of the date of filing this application.

In some embodiments, the double-stranded region of a double-stranded iRNA agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a double-stranded iRNA agent is equal to or at least 14, 15, 16, 17, 18, 19, 20, 21, 22, 23, 23, 24, 25, 26, 27, 28, 29, or 30 nucleotides in length.

In some embodiments, the sense strand of a double-stranded iRNA agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the double-stranded iRNA agent are each independently 15 to 30 nucleotides in length.

In one embodiment, the sense and antisense strands of the double-stranded iRNA agent are each independently 19 to 25 nucleotides in length.

In one embodiment, the sense and antisense strands of the double-stranded iRNA agent are each independently 21 to 23 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single stranded overhangs at the 3′-end.

In one aspect of the invention, an agent for use in the methods and compositions of the invention is a single-stranded antisense nucleic acid molecule that inhibits a target mRNA via an antisense inhibition mechanism. The single-stranded antisense RNA molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense RNA molecule may be about 15 to about 30 nucleotides in length and have a sequence that is complementary to a target sequence. For example, the single-stranded antisense RNA molecule may comprise a sequence that is at least about 15, 16, 17, 18, 19, 20, or more contiguous nucleotides from any one of the antisense sequences described herein.

In one embodiment, at least partial suppression of the expression of a target gene, is assessed by a reduction of the amount of target mRNA which can be isolated from or detected in a first cell or group of cells in which a target gene is transcribed and which has or have been treated such that the expression of a target gene is inhibited, as compared to a second cell or group of cells substantially identical to the first cell or group of cells but which has or have not been so treated (control cells). The degree of inhibition may be expressed in terms of.

$$\frac{( mRNA in control cells ) - ( mRNA in treated cells )}{( mRNA in control cells )} 100 \%$$

In one embodiment, inhibition of expression is determined by the dual luciferase method wherein the RNAi agent is present at 10 nM.

The phrase “contacting a cell with an RNAi agent,” such as a dsRNA, as used herein, includes contacting a cell by any possible means. Contacting a cell with an RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, or to the bloodstream or the subcutaneous space, such that the agent will subsequently reach the tissue where the cell to be contacted is located. In some embodiments, the RNAi agent may contain or be coupled to a ligand, e.g., one or more alpha-v-beta-6 (αvβ6) integrin targeting ligand.

An “alpha-v-beta-6 (αvβ6) integrin targeting ligand”, as used herein, includes any moiety (e.g., peptides and small molecules) which bind an αvβ6 integrin and are able to mediate delivery of a dsRNA agent to which they are attached to skeletal muscle (e.g., skeletal muscle cell or skeletal muscle tissue) and/or cardiac muscle (e.g., cardiac muscle cell or cardiac muscle tissue). The αvβ6 integrin targeting ligands bind αvβ6 integrin or the αvβ6 integrin receptor on skeletal muscle/cardiac myocytes (cells). Exemplary αvβ6 integrin targeting ligands are described in Section II below.

In one embodiment, contacting a cell with an RNAi agent includes “introducing” or “delivering the RNAi agent into the cell” by facilitating or effecting uptake or absorption into the cell. Absorption or uptake of a RNAi agent can occur through unaided diffusive or active cellular processes, or by auxiliary agents or devices. Introducing a RNAi agent into a cell may be in vitro or in vivo. For example, for in vivo introduction, a RNAi agent can be injected into a tissue site or administered systemically. In vitro introduction into a cell includes methods known in the art such as electroporation and lipofection. Further approaches are described herein below or are known in the art.

As used herein, a “subject” is an animal, such as a mammal, including a primate (such as a human, a non-human primate, e.g., a monkey, and a chimpanzee), or a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In one embodiment, the subject is a human, such as a human being treated or assessed for a disease, disorder, or condition that would benefit from reduction in target gene expression; a human at risk for a disease, disorder, or condition that would benefit from reduction in target gene expression; a human having a disease, disorder, or condition that would benefit from reduction in target gene expression; or human being treated for a disease, disorder, or condition that would benefit from reduction in target gene expression as described herein. In some embodiments, the subject is a female human. In other embodiments, the subject is a male human. In one embodiment, the subject is an adult subject. In another embodiment, the subject is a pediatric subject.

As used herein, the terms “treating” or “treatment” refer to a beneficial or desired result including, but not limited to, alleviation or amelioration of one or more signs or symptoms associated with target gene expression or target gene protein production, e.g., a target gene-associated disease, e.g., a muscle disorder, e.g., a skeletal muscle disorder, and/or a cardiac muscle disorder, or symptoms associated with unwanted target gene expression; diminishing the extent of unwanted target activation or stabilization; amelioration or palliation of unwanted target activation or stabilization. “Treatment” can also mean prolonging survival as compared to expected survival in the absence of treatment.

The term “lower” in the context of the level of a target gene in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of a target gene in a subject is a decrease to a level accepted as within the range of normal for an individual without such disorder. In certain embodiments, the expression of the target is normalized, i.e., decreased towards or to a level accepted as within the range of normal for an individual without such disorder, e.g., blood glucose level, blood uric acid level, blood lipid level, blood oxygen level, white blood cell count, kidney function, spleen function, liver function. For example, chronic hyperuricemia is defined as serum urate levels greater than 6.8 mg/dl (greater than 360 mmol/), the level above which the physiological saturation threshold is exceeded (Mandell, Cleve. Clin. Med. 75:S5-S8, 2008). As used here, “lower” in a subject can refer to lowering of gene expression or protein production in a cell in a subject does not require lowering of expression in all cells or tissues of a subject. For example, as used herein, lowering in a subject can include lowering of gene expression or protein production in a subject.

The term “lower” can also be used in association with normalizing a symptom of a disease or condition, i.e. decreasing the difference between a level in a subject suffering from a target gene-associated disease towards or to a level in a normal subject not suffering from a target gene-associated disease. As used herein, if a disease is associated with an elevated value for a symptom, “normal” is considered to be the upper limit of normal. If a disease is associated with a decreased value for a symptom, “normal” is considered to be the lower limit of normal.

As used herein, “prevention” or “preventing,” when used in reference to a disease, disorder, or condition thereof, that would benefit from a reduction in expression of a target gene or production of a target protein, refers to a reduction in the likelihood that a subject will develop a symptom associated with such a disease, disorder, or condition, e.g., a symptom of a target gene-associated disease. The failure to develop a disease, disorder, or condition, or the reduction in the development of a symptom associated with such a disease, disorder, or condition (e.g., by at least about 10% on a clinically accepted scale for that disease or disorder), or the exhibition of delayed symptoms delayed (e.g., by days, weeks, months or years) is considered effective prevention.

As used herein, the term “target gene-associated disease,” is a disease or disorder that would benefit from reduction in the expression or activity of the target gene. The term “target gene-associated disease,” is a disease or disorder that is caused by, or associated with expression or protein production of the target gene. The term “target gene-associated disease” includes a disease, disorder or condition that would benefit from a decrease in expression or protein activity of the target gene. Additional information regarding specific target genes and disease that would benefit from reduction in expression of the target gene are described below.

In one embodiment, the target gene-associated disease is a muscle disorder.

Exemplary muscle disorders include Myostatin-related muscle hypertrophy, congenital myasthenic syndrome, facioscapulohumeral muscular dystrophy (FSHD), Spinal Muscular Atrophy (SMA), Myotonic Dystrophy Type 1 (DM1), Pompe disease, PLN cardiomyopathy, spasticity, obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); hypertrophic cardiomyopathy (HCM); and PLN cardiomyopathy.

In one embodiment, the target gene-associated disease is a skeletal muscle disease or disorder.

In one embodiment, the target gene-associated disease is a cardiac muscle disease or disorder.

Exemplary cardiac muscle disorders include obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); hypertrophic cardiomyopathy (HCM); and PLN cardiomyopathy.

“Heart failure” (“HF”) or “congestive heart failure” (“CHF”) is a chronic condition in which the heart doesn't pump blood as well as it should. Heart failure occurs when the heart's capacity to pump blood cannot keep up with the body's need. Heart failure can occur if the heart cannot pump (systolic) or fill (diastolic) adequately. As the heart weakens, blood begins to back up and force liquid through the capillary walls. The term “congestive” refers to the resulting buildup of fluid in the ankles and feet, arms, lungs, and/or other organs.

One type of heart failure is “heart failure with preserved left ventricular function” (“HF-pEF”) also known as “heart failure with preserved ejection fraction” (“HF-pEF”) is a condition in which the heart contracts and pumps normally, but the ventricles are thicker and stiffer than normal. Because of this, the ventricles can't relax properly and fill up all the way. Because there's less blood in the ventricles, less blood is pumped out to the rest of the body when the heart contracts.

The most common cause of congestive heart failure is coronary artery disease. Risk factors for coronary artery disease include high levels of cholesterol and/or triglyceride, high blood pressure, poor diet, a sedentary lifestyle, diabetes, smoking, being overweight or obese, and stress. In addition to coronary artery disease, several other conditions can damage the heart muscles, including inherited and genetic factors, some infections and autoimmune diseases and some treatments such as chemotherapy.

Symptoms of CHF include shortness of breath, fatigue, swollen legs, and rapid heartbeat.

Treatments can include eating less salt, limiting fluid intake, and taking prescription medications, e.g., vasodilators, diuretics, aldosterone inhibitors, ACE inhibitors or ARB drugs, digitalis glycosides, anticoagulants or antiplatelets, beta-blockers, and tranquilizers, and surgical procedures, include for example, bypass surgery, heart valve replacement, implantation of a pacemaker, e.g., biventricular pacing therapy or an implantable cardioverter defibrillator, ventricular assist devices (VAD therapy), and heart transplant.

“Hypertrophic cardiomyopathy” (“HCM”) refers to impaired heart function associated with abnormally thick heart muscle in the absence of other heart disease; e.g., valvular heart disease. “Hypertrophic obstructive cardiomyopathy” (“HOCM”) is a subtype of HCM, where the wall (septum) between the two bottom chambers of the heart thickens. The walls of the pumping chamber can also become stiff. The thickened septum may cause a narrowing that can block or reduce the blood flow from the left ventricle to the aorta, which is a condition called “outflow tract obstruction.”

Both HCM and HOCM may be caused by heart muscle gene mutation, which may be inherited. As such, multiple family members may be affected by HCM and HOCM. Phenotypic expression of the gene mutation may be variable.

Both HCM and HOCM may be caused by heart muscle gene mutation, which may be inherited. As such, multiple family members may be affected by HCM and HOCM. Phenotypic expression of the gene mutation may be variable. In other words, even with the same gene mutation, the severity of heart function impairment may vary between affected patients.

Symptoms associated with HCM may vary in severity and character as well, including, fatigue, chest pain, dyspnea, abnormal heart rhythm, heart failure, syncope, and sudden cardiac death.

Treatments include pacemakers, defibrillators, alcohol septal ablation, surgical myectomy, advanced heart failure therapy, beta blockers, calcium channel blockers, and anti-arrhythmics.

“Familial hypertrophic cardiomyopathy” is an autosomal dominant disease characterized mainly by left ventricular hypertrophy. Thickening usually occurs in the interventricular septum. In some, thickening of the interventricular septum impedes the flow of oxygen-rich blood from the heart, which may lead to an abnormal heart sound during a heartbeat (heart murmur) and other signs and symptoms of the condition. Other affected individuals do not have physical obstruction of blood flow, but the pumping of blood is less efficient, which can also lead to symptoms of the condition. Cardiac hypertrophy often begins in adolescence or young adulthood, although it can develop at any time throughout life.

The symptoms of familial hypertrophic cardiomyopathy are variable, even within the same family. Many affected individuals have no symptoms. Other people with familial hypertrophic cardiomyopathy may experience chest pain; shortness of breath, especially with physical exertion; a sensation of fluttering or pounding in the chest (palpitations); lightheadedness; dizziness; and fainting.

While most people with familial hypertrophic cardiomyopathy are symptom-free or have only mild symptoms, this condition can have serious consequences. It can cause abnormal heart rhythms (arrhythmias) that may be life threatening. People with familial hypertrophic cardiomyopathy have an increased risk of sudden death, even if they have no other symptoms of the condition. A small number of affected individuals develop potentially fatal heart failure, which may require heart transplantation.

Mutations in one of several genes can cause familial hypertrophic cardiomyopathy; the most commonly involved genes are MYH7, MYBPC3, TNNT2, and TNNI3. Other genes, including some that have not been identified, may also be involved in this condition.

Treatments include, beta blockers, calcium channel blockers, heart rhythm drugs such as amiodarone (Pacerone) or disopyramide (Norpace), and blood thinners such as warfarin (Coumadin, Jantoven), dabigatran (Pradaxa), rivaroxaban (Xarelto) or apixaban (Eliquis). Surgeries or other procedures include apical myectomy, septal myectomy, septal ablation, and implantable cardioverter-defibrillator (ICD).

“Atrial fibrillation” (“AFIB”) is when the atria beat chaotically and irregularly—out of coordination with the ventricles. The result is a fast and irregular heart rhythm. The heart rate in atrial fibrillation may range from 100 to 175 beats a minute. The normal range for a heart rate is 60 to 100 beats a minute.

Episodes of atrial fibrillation may come and go, or may go away and may require treatment. Although atrial fibrillation itself usually isn't life-threatening, it is a serious medical condition that sometimes requires emergency treatment.

A major concern with atrial fibrillation is the potential to develop blood clots within the atria which may circulate to other organs and lead to blocked blood flow (ischemia).

Causes of AFIB include, abnormalities or damage to the heart's structure, high blood pressure, heart attack, coronary artery disease, abnormal heart valves, congenital heart defects, an overactive thyroid gland or other metabolic imbalance, exposure to stimulants, such as medications, caffeine, tobacco or alcohol, sick sinus syndrome—improper functioning of the heart's natural pacemaker, lung diseases, previous heart surgery, viral infections, stress due to surgery, pneumonia or other illnesses, and sleep apnea.

Symptoms include palpitations, which are sensations of a racing, uncomfortable, irregular heartbeat or a flip-flopping in the chest, weakness, reduced ability to exercise, fatigue, lightheadedness, dizziness, shortness of breath, and chest pain.

Treatments include, electrical cardioversion, anti-arrhythmics, digoxin, beta blockers, calcium channel blockers anticoagulants, catheter ablation, Maze procedure, atrioventricular (AV) node ablation, and left atrial appendage closure.

“Ventricular fibrillation” (“VFIB”) is a type of abnormal heart rhythm (arrhythmia). During ventricular fibrillation, disorganized heart signals cause the ventricles to twitch (quiver) uselessly. As a result, the heart doesn't pump blood to the rest of the body.

Ventricular fibrillation is an emergency that requires immediate medical attention. It's the most frequent cause of sudden cardiac death.

Collapse and loss of consciousness is the most common symptom of ventricular fibrillation. Other symptoms include chest pain, very fast heartbeat (tachycardia), dizziness, nausea, and shortness of breath.

Risk factors include previous episode of ventricular fibrillation, previous heart attack, a congenital heart defect, heart muscle disease (cardiomyopathy), injuries that cause damage to the heart muscle, such as being struck by lightning, drug misuse, especially with cocaine or methamphetamine, and severe imbalance of potassium or magnesium.

Treatments include, cardiopulmonary resuscitation (CPR), defibrillation, anti-arrhythmics, an implantable cardioverter-defibrillator (ICD), cardiac ablation, coronary angioplasty and stent placement, and coronary bypass surgery.

A “myocardial infarction” or “MI” occurs when the flow of blood to the heart is blocked. The blockage is most often a buildup of fat, cholesterol and other substances, which form a plaque in the arteries that feed the heart (coronary arteries).

Symptoms include pressure, tightness, pain, or a squeezing or aching sensation in the chest or arms that may spread to the neck, jaw or back, nausea, indigestion, heartburn or abdominal pain, shortness of breath, cold sweat, fatigue, lightheadedness or sudden dizziness

Heart attack risk factors include age (e.g., men age 45 or older and women age 55 or older are more likely to have a heart attack than are younger men and women, tobacco, high blood pressure. Over time, high blood pressure can damage arteries that lead to your heart. High blood pressure that occurs with other conditions, such as obesity, high cholesterol or diabetes, increases your risk even more, high cholesterol or triglyceride levels, obesity, diabetes, metabolic syndrome, family history of heart attacks, lack of physical activity, stress, illicit drug use, a history of preeclampsia, and an autoimmune condition.

Treatments include, aspirin, thrombolytics, antiplatelet agents, other blood-thinning medications, pain relievers, nitroglycerin, beta blockers, ACE inhibitors, statins, coronary angioplasty and stenting, and coronary artery bypass surgery.

“Supraventricular tachycardia” (“SVT”) is as an abnormally fast or erratic heartbeat that affects the heart's atria. During an episode of SVT, the heart beats about 150 to 220 times per minute, but it can occasionally beat faster or slower.

The main symptom of supraventricular tachycardia (SVT) is a very fast heartbeat (100 beats a minute or more) that may last for a few minutes to a few days. The fast heartbeat may come and go suddenly, with stretches of normal heart rates in between.

Signs and symptoms of supraventricular tachycardia may include very fast (rapid) heartbeat, a fluttering or pounding in the chest (palpitations), a pounding sensation in the neck, weakness or feeling very tired (fatigue), chest pain, shortness of breath, lightheadedness or dizziness, sweating, and fainting (syncope) or near fainting. Some with SVT have no signs or symptoms at all.

For some, a supraventricular tachycardia episode is related to an obvious trigger, such as exercise, stress or lack of sleep. Some people may not have a noticeable trigger. Things that may cause an SVT episode include age, coronary artery disease, previous heart surgery, heart disease, heart failure, other heart problems, such as Wolff-Parkinson-White syndrome, chronic lung disease, consuming too much caffeine, drinking too much alcohol, drug use, particularly stimulants such as cocaine and methamphetamines, pregnancy, smoking, thyroid disease, tobacco, sleep apnea, diabetes, and certain medications, including asthma medications and over-the-counter cold and allergy drugs.

Treatments include, carotid sinus massage, vagal maneuvers, cardioversion, beta blockers, anti-arrhythmics, calcium channel blocker, catheter ablation, and pacemaker.

“Hypertrophic cardiomyopathy” (“HCM”) is a disease in which the heart muscle becomes abnormally thick (hypertrophied). The thickened heart muscle can make it harder for the heart to pump blood.

“Angina” is a type of chest pain caused by reduced blood flow to the heart. Angina is a symptom of coronary artery disease.

Angina, also called angina pectoris, is often described as squeezing, pressure, heaviness, tightness or pain in your chest. Some with angina symptoms say angina feels like a vise squeezing their chest or a heavy weight lying on their chest. There may also be pain in the arms, neck, jaw, shoulder or back. Other symptoms that you may have with angina include dizziness, fatigue, nausea, shortness of breath, and sweating.

Risk factors include tobacco, diabetes, high blood pressure, high cholesterol or triglyceride levels, family history of heart disease, age (e.g., men older than 45 and women older than 55 have a greater risk than do younger adults), lack of exercise, obesity, and stress.

Treatments include, lifestyle changes, nitrates, aspirin, clot-preventing drugs, beta blockers, statins, calcium channel blockers, blood pressure-lowering medications, angiotensin-converting enzyme (ACE) inhibitors or angiotensin II receptor blockers (ARBs), ranolazine (Ranexa), angioplasty and stenting, coronary artery bypass surgery, and external counterpulsation (ECP).

Phospholamban (PLN) cardiomyopathy, as used herein, refers to a specific subtype of hereditary cardiomyopathy caused by PLN p.(Arg14del), a pathogenic variant in the gene encoding PLN, which is a protein with a central role in calcium homeostasis in cardiac tissue. This protein ensures proper contraction and relaxation of the human heart. Carriers of this pathogenic variant have a high risk of developing dilated cardiomyopathy (DCM), arrhythmic cardiomyopathy (ACM), or both. They commonly have a high arrhythmic burden, with premature ventricular contractions (PVCs) and ventricular tachycardia, remarkable low-voltage electrocardiograms (ECGs), left ventricular dysfunction, and a positive family history for sudden cardiac death. PLN p.(Arg14del) cardiomyopathy has been found in several European countries, but also in the United States, Canada, and China. On a global scale it is a rare disease, but it is particularly common in The Netherlands, with the pathogenic variant being present in 12% of all ACM patients and 15% of all DCM patients.

Exemplary skeletal muscle disorders include Myostatin-related muscle hypertrophy, congenital myasthenic syndrome, facioscapulohumeral muscular dystrophy (FSHD), Spinal Muscular Atrophy (SMA), Myotonic Dystrophy Type 1 (DM1), Pompe disease, PLN cardiomyopathy, and spasticity.

Myostatin-related muscle hypertrophy is a rare condition characterized by reduced body fat and increased muscle size. Affected individuals have up to twice the usual amount of muscle mass in their bodies. They also tend to have increased muscle strength. Myostatin-related muscle hypertrophy is caused by mutations in the MSTN gene. It follows an incomplete autosomal dominant pattern of inheritance.

Congenital myasthenic syndromes (CMS) are a heterogeneous group of early-onset genetic neuromuscular transmission disorders due to mutations in proteins involved in the organisation, maintenance, function, or modification of the motor endplate (endplate myopathies), e.g., CHRNA1, CHRNB1, CHRBD, CHRNE, CHRNG, COL13A1, DOX7, LRP4, MUSK, RAPSN, or SCN4A. CMS are clinically characterised by abnormal fatigability, or transient or permanent weakness of extra-ocular, facial, bulbar, truncal, respiratory, or limb muscles. Onset of endplate myopathy is intrauterine, congenital, in infancy, or childhood, and rarely in adolescence. Severity ranges from mild, phasic weakness, to disabling, permanent muscle weakness, respiratory insufficiency, and early death. All subtypes of CMS share the clinical features of fatigability and muscle weakness, but age of onset, presenting symptoms, and response to treatment vary depending on the molecular mechanism that results from the underlying genetic defect. The term CMS is misleading since not all CMS are congenital. See, Finsterer (2019) Orphanet J Rare Dis. 14: 57 for a review.

Facioscapulohumeral muscular dystrophy (FSHD) type 1 is an autosomal dominant condition caused by mutations in DUX4. FSHD typically presents before age 20 years with weakness of the facial muscles and the stabilizers of the scapula or the dorsiflexors of the foot. There is extreme clinical variability. In some cases, Congenital facial weakness may be present. In FSHD, the muscle weakness is slowly progressive and approximately 20% of affected individuals eventually require a wheelchair. Life expectancy is not shortened. The incidence is approximately 4 individuals affected per 100,000 people.

Spinal muscular atrophy, as used herein, refers to a genetic disorder characterized by weakness and wasting (atrophy) in muscles used for movement (skeletal muscles). It is caused by a loss of specialized nerve cells, called motor neurons that control muscle movement. The weakness tends to be more severe in the muscles that are close to the center of the body (proximal) compared to muscles away from the body's center (distal). The muscle weakness usually worsens with age. There are many types of spinal muscular atrophy that are caused by changes in the same genes. The types differ in age of onset and severity of muscle weakness; however, there is overlap between the types. Other forms of spinal muscular atrophy and related motor neuron diseases, such as spinal muscular atrophy with progressive myoclonic epilepsy, spinal muscular atrophy with lower extremity predominance, X-linked infantile spinal muscular atrophy, and spinal muscular atrophy with respiratory distress type 1 are caused by mutations in other genes.

Mutations in the SMN1 gene cause all types of spinal muscular atrophy described above. The number of copies of the SMN2 gene modifies the severity of the condition and helps determine which type develops. The SMN1 and SMN2 genes both provide instructions for making a protein called the survival motor neuron (SMN) protein. Normally, most functional SMN protein is produced from the SMN1 gene, with a small amount produced from the SMN2 gene. Several different versions of the SMN protein are produced from the SMN2 gene, but only one version is functional; the other versions are smaller and quickly broken down. The SMN protein is one of a group of proteins called the SMN complex, which is important for the maintenance of motor neurons. Motor neurons transmit signals from the brain and spinal cord that tell skeletal muscles to tense (contract), which allows the body to move.

Myotonic dystrophy, as used herein, refers to a part of a group of inherited disorders called muscular dystrophies. It is the most common form of muscular dystrophy that begins in adulthood. Myotonic dystrophy is characterized by progressive muscle wasting and weakness. People with this disorder often have prolonged muscle contractions (myotonia) and are not able to relax certain muscles after use. Other signs and symptoms of myotonic dystrophy include clouding of the lens of the eye (cataracts) and abnormalities of the electrical signals that control the heartbeat (cardiac conduction defects). Some affected individuals develop a condition called diabetes mellitus, in which blood sugar levels can become dangerously high. The features of myotonic dystrophy often develop during a person's twenties or thirties, although they can occur at any age. The severity of the condition vanes widely among affected people, even among members of the same family. There are two major types of myotonic dystrophy: type 1 and type 2. Their signs and symptoms overlap, although type 2 tends to be milder than type 1. The muscle weakness associated with type 1 particularly affects muscles farthest from the center of the body (distal muscles), such as those of the lower legs, hands, neck, and face. Muscle weakness in type 2 primarily involves muscles close to the center of the body (proximal muscles), such as the those of the neck, shoulders, elbows, and hips. The two types of myotonic dystrophy are caused by mutations in different genes.

Myotonic dystrophy type 1 is caused by mutations in the DMPK gene, while type 2 results from mutations in the CNBP gene. The protein produced from the DMPK gene likely plays a role in communication within cells. It appears to be important for the correct functioning of cells in the heart, brain, and skeletal muscles (which are used for movement). The protein produced from the CNBP gene is found primarily in the heart and in skeletal muscles, where it helps regulate the function of other genes. Similar changes in the structure of the DMPK and CNBP genes cause myotonic dystrophy type 1 and type 2. In each case, a segment of DNA is abnormally repeated many times, forming an unstable region in the gene. The gene with the abnormal segment produces an unusually long messenger RNA, which is a molecular blueprint of the gene that guides the production of proteins. The unusually long messenger RNA forms clumps inside the cell that interfere with the production of many other proteins. These changes prevent muscle cells and cells in other tissues from functioning normally, which leads to the signs and symptoms of myotonic dystrophy. If these changes affect the DMPK gene, the result is myotonic dystrophy type 1, if the CNBP gene is affected, the result is myotonic dystrophy type 2.

Pompe disease, as used herein, refers to an inherited disorder caused by the buildup of a complex sugar called glycogen in the body's cells. The accumulation of glycogen in certain organs and tissues, especially muscles, impairs their ability to function normally. There are three types of Pompe disease, which differ in severity and the age at which they appear. These types are known as classic infantile-onset, non-classic infantile-onset, and late-onset. The classic form of infantile-onset Pompe disease begins within a few months of birth. Infants with this disorder typically experience muscle weakness (myopathy), poor muscle tone (hypotonia), an enlarged liver (hepatomegaly), and heart defects. Affected infants may also fail to gain weight and grow at the expected rate (failure to thrive) and have breathing problems. If untreated, this form of Pompe disease leads to death from heart failure in the first year of life. The non-classic form of infantile-onset Pompe disease usually appears by age 1. It is characterized by delayed motor skills (such as rolling over and sitting) and progressive muscle weakness. The heart may be abnormally large (cardiomegaly), but affected individuals usually do not experience heart failure. The muscle weakness in this disorder leads to serious breathing problems, and most children with non-classic infantile-onset Pompe disease live only into early childhood. The late-onset type of Pompe disease may not become apparent until later in childhood, adolescence, or adulthood. Late-onset Pompe disease is usually milder than the infantile-onset forms of this disorder and is less likely to involve the heart. Most individuals with late-onset Pompe disease experience progressive muscle weakness, especially in the legs and the trunk, including the muscles that control breathing. As the disorder progresses, breathing problems can lead to respiratory failure.

Mutations in the GAA gene cause Pompe disease. The GAA gene provides instructions for producing an enzyme called acid alpha-glucosidase (also known as acid maltase). This enzyme is active in lysosomes, which are structures that serve as recycling centers within cells. The enzyme normally breaks down glycogen into a simpler sugar glucose, which is the main energy source for most cells. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.

Spasticity, as used herein, refers to a condition in which muscles stiffen or tighten, preventing normal fluid movement. The muscles remain contracted and resist being stretched, thus affecting movement, speech and gait. Spasticity is generally caused by damage or disruption to the area of the brain and spinal cord that are responsible for controlling muscle and stretch reflexes. These disruptions can be due to an imbalance in the inhibitory and excitatory signals sent to the muscles, causing them to lock in place. Spasticity can be harmful to growing children as it can affect muscles and joints. People with brain injury, spinal cord injury, cerebral palsy or multiple sclerosis can have varying degrees of spasticity.

“Therapeutically effective amount,” as used herein, is intended to include the amount of an RNAi agent that, when administered to a subject having a target gene-associated disease, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of a RNAi agent that, when administered to a subject having a target gene-associated disorder, e.g., gout or diabetes, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of a RNAi agent that produces some desired local or systemic effect at a reasonable benefit/risk ratio applicable to any treatment. A RNAi agent employed in the methods of the present disclosure may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations. Pharmaceutically acceptable carriers for pulmonary delivery are known in the art and will vary depending on the desired location for deposition of the agent, e.g., upper or lower respiratory system, and the type of device to be used for delivery, e.g., sprayer, nebulizer, dry powder inhaler.

“Pharmaceutically acceptable salts” of each of RNAi agents herein include, but are not limited to, a sodium salt, a calcium salt, a lithium salt, a potassium salt, an ammonium salt, a magnesium salt, an mixtures thereof. One skilled in the art will appreciate that the RNAi agent, when provided as a polycationic salt having one cation per free acid group of the optionally modified phosphodiester backbone and/or any other acidic modifications (e.g., 5′-terminal phosphonate groups). For example, an oligonucleotide of “n” nucleotides in length contains n−1 optionally modified phosphodiesters, so that an oligonucleotide of 21 nt in length may be provided as a salt having up to 20 cations (e.g., 20 sodium cations). Similarly, an RNAi agents having a sense strand of 21 nt in length and an antisense strand of 23 nt in length may be provided as a salt having up to 42 cations (e.g., 42 sodium cations). In the preceding example, where the RNAi agent also includes a 5′-terminal phosphate or a 5′-terminal vinylphosphonate group, the RNAi agent may be provided as a salt having up to 44 cations (e.g., 44 sodium cations).

The term “sample,” as used herein, includes a collection of similar fluids, cells, or tissues isolated from a subject, as well as fluids, cells, or tissues present within a subject. Examples of biological fluids include blood, serum and serosal fluids, plasma, bronchial fluids, sputum, cerebrospinal fluid, ocular fluids, lymph, urine, saliva, sputum, and the like. Tissue samples may include samples from tissues, organs or localized regions. For example, samples may be derived from particular organs, parts of organs, or fluids or cells within those organs.

### II. Modified Alpha-v-Beta-6 (αvβ6) Integrin Compounds and Ligands

Integrins are cell surface receptors that, upon ligand binding, activate signal transduction pathways including signaling pathways involved in cytoskeleton organization and cell cycle regulation. Integrins are also involved in cell attachment to the extracellular matrix and the integrin ligands comprise common extracellular matrix components, including fibronectin, collagen, laminin, fibrinogen, thrombospondin, and glycoproteins (e.g., tenascin C, osteopontin, and nefronectin). In humans there are at least twenty-four known integrin heterodimers composed of an alpha and a beta subunit, e.g., αvβ6. It is the combination of the alpha and beta subunits which determines the ligand specificity and function of the integrin. Nearly all cells express at least one integrin and the expression and/or activity of integrins can be influenced by other signal inducing molecules, such as cytokines or steroids.

The main function of αvβ6 is the activation of cytokine transforming growth factor-b1 (TGF-β1). Latent-TGF-β1 is bound to the extracellular matrix, covered by its pro-peptide latency associated peptide (LAP). αvβ6 binds LAP, and through cytoskeletal force releases TGF-β1. TGF-β1 regulates multiple processes including cell proliferation, differentiation, angiogenesis, epithelial-mesenchymal-transition (EMT) and immune suppression. These processes combine to heal wounds but when uncontrolled can promote tissue pathologies.

The present invention provides αvβ6 compounds and ligands comprising such αvβ6 compounds that can be conjugated to, e.g., a dsRNA agent, for efficient extrahepatic delivery of the dsRNA agent.

A. Alpha-v-Beta-6 (αvβ6) Integrin Compounds

In one aspect, the present application provides modified integrin-modifying compounds in a form suitable for conjugation to an oligonucleotide, either directly or via a carrier group. In some embodiments, the present disclosure provides a compound of the Formula (IV),

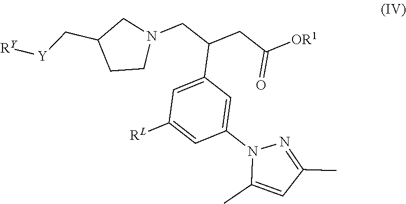

or a salt thereof, wherein:
Y is O, N(H), S, or CH₂; (e.g., O or CH₂)
R¹ is hydrogen or C₁₋₆alkyl (e.g., methyl);
R^{Y} is

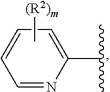

wherein
   m is 0, 1, 2, 3, or 4; and
   each R² is independently R, or two R² groups on adjacent carbon atoms taken together with the atoms to which they are bound form a fused 4-8 membered ring that is optionally substituted by 1, 2, 3 or 4 groups independently selected from group consisting of R and a nitrogen protecting group;
and
R^{L} is —N(R³)(R⁴), —O(R⁵), —S(R⁵), or —R⁵, wherein
   R³ and R⁴ are either
      (i) R³ is hydrogen or C₁₋₆alkyl and R⁴ is R⁵; or
      (ii) R³ and R⁴ taken together with the nitrogen atom to which they are attached form a 4-8 membered monocyclic heterocyclyl group that is substituted with R⁵;
   and
   R⁵ is -L-Z wherein
      L is -L¹-[G-L²]_{q}-G-L³-*, wherein
         * is the bond to Z;
         q is 0 or an integer selected from 1-25; (e.g., 1-20, or 1-15);
         L¹ is a bond or -B-A-;
         each L² is independently -A-B-A-;
         L³ is a bond or -A-B-A-;
         each G is independently -D-E-F-, wherein D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
         each A is independently a bond, —O—, —S—, or —N(R^{N})—.
         each B is independently a bond, CH₂, C(O), C(S), C(NR^{N}), (O), S(O)₂, P(O)(OH), P(S)(OH), or P(S)(SH);
         each R^{N} is independently hydrogen or C₁₋₆alkyl, or two R^{N} within an -A-B-A- group taken together with the atoms to which they are connected from a 4-8 membered heterocyclyl;
         and
      Z is one member of a reactive pair;
and
each R group is independently selected from the group consisting of R′, C₁₋₆alkyl, C₁₋₆ haloalkyl, C₂₋₆alkenyl, C₂₋₆alkynyl, C₃₋₈cycloalkyl, heterocyclyl, aryl, heteroaryl, C₃₋₈cycloalkylC₁₋₆alkyl, heterocyclylC₁₋₆alkyl, aryl C₁₋₆alkyl, heteroarylC₁₋₆alkyl, each of which, other than R′, is optionally substituted with 1, 2, or 3 R′ groups, wherein
   each R′ is independently halogen, cyano, azido, nitro, —N(R^{b})₂, —O(R^{a}), —S(R⁰), —C(O)OR⁰,
   C(O)R⁰, —C(O)N(R⁰)₂, —C(NR⁰)OR, —C(NR⁰)R⁰, —C(NR⁰)N(R⁰)₂, —C(S)OR⁰, —C(S)R⁰, —C(S)N(R⁰)₂, —S(O)₂R⁰, —S(O)₂OR⁰, —S(O)₂N(R⁰)₂, —N(R⁰)C(O)OR⁰, —N(R⁰)C(O)R⁰, —N (R⁰)C(O)N(R⁰)₂, —N(R⁰)S(O)₂R⁰, —N(R⁰)S(O)₂OR⁰, —N(R⁰)S(O)₂N(R⁰)₂, —OC(O)OR⁰, —OC(O)R⁰, —OC(O)N(R⁰)₂, —OS(O)₂R⁰, —OS(O)₂OR⁰, —OS(O)₂N(R⁰)₂, or —SC(O)R⁰, wherein
      each R⁰ is independently hydrogen or C₁₋₆alkyl; each R^{a} is independently hydrogen, C₁₋₆alkyl, or a hydroxyl protecting group; and each R^{b} is independently hydrogen, C₁₋₆alkyl, or a nitrogen protecting group
provided that in each -D-E-F- group, at least one of D, E, and F is not a bond; and R^{L} is not N-morpholinyl.

In some embodiments, in —N(R³)(R⁴), R³ and R⁴ do not form a morpholino ring.

In some embodiments, in each -A-B-A- group, B is only a bond when one of the A groups is not a bond.

In some embodiments, each R group is independently selected from the group consisting of R′, C₁₋₆alkyl, C₁₋₆haloalkyl, C₂₋₆alkenyl, C₂₋₆alkynyl, C₃₋₈cycloalkyl, heterocyclyl, aryl, heteroaryl, C₃₋₈cycloalkylC₁₋₆alkyl, heterocyclylC₁₋₆alkyl, aryl C₁₋₆alkyl and heteroaryl₁₋₆alkyl, each of which, other than R′, is optionally substituted with 1, 2, or 3 R′ groups, wherein each R′ is independently halogen, cyano, azido, nitro, —N(R^{b})₂, —O(R^{a}), —S(R⁰), —C(O)OR⁰, —C(O)R⁰, —C(O)N(R⁰)₂, —N(R⁰)C(O)R⁰, —OC(O)OR⁰, or —OC(O)R⁰, wherein each R⁰ is independently hydrogen or C₁₋₆alkyl; each R^{a} is independently hydrogen, C₁₋₆alkyl, or a hydroxyl protecting group; and each R^{b} is independently hydrogen, C₁₋₆alkyl, or a nitrogen protecting group.

In some embodiments, each R group is independently selected from the group consisting of R′, C₁₋₆alkyl, C₁₋₆haloalkyl, C₂₋₆alkenyl, C₂₋₆alkynyl, C₃₋₆cycloalkyl, 5-6 membered heterocyclyl, phenyl, 5-6 membered heteroaryl, and benzyl, each of which, other than R′, is optionally substituted with 1, 2, or 3 R′ groups, wherein each R′ is independently halogen, cyano, azido, nitro, —N(R^{b})₂, —O(R^{a}), —S(R⁰), —C(O)OR⁰, —C(O)R⁰, —C(O)N(R⁰)₂, —N(R⁰)C(O)R⁰, —OC(O)OR, or —OC(O)R⁰, wherein each R⁰ is independently hydrogen or C₁₋₆alkyl; each R^{a} is independently hydrogen, C₁₋₆alkyl, or a hydroxyl protecting group; and each R^{b} is independently hydrogen, C₁₋₆alkyl, or a nitrogen protecting group.

In some embodiments, each R group is independently selected from the group consisting of R′, C₁₋₆alkyl, C₃₋₆cycloalkyl, 5-6 membered heterocyclyl, phenyl, 5-6 membered heteroaryl, and benzyl, each of which, other than R′, is optionally substituted with 1, 2, or 3 R′ groups, wherein each R′ is independently halogen, cyano, azido, nitro, —N(R^{b})₂, —O(R^{a}), —S(R⁰), —C(O)OR⁰, —C(O)R⁰, —C(O)N(R⁰)₂, —N(R⁰)C(O)R⁰, —OC(O)OR⁰, or —OC(O)R⁰, wherein each R⁰ is independently hydrogen or C₁₋₆alkyl; each R^{a} is independently hydrogen, C₁₋₆alkyl, or a hydroxyl protecting group; and each R^{b} is independently hydrogen, C₁₋₆alkyl, or a nitrogen protecting group.

R^{Y} Embodiments, Formulae (IV) and (IV-a) through (IV-b)

In some embodiments of any one of Formula (IV) and Formulae (IV-a) through (IV-b), in R^{Y}, each R² is independently R as defined in Formula (IV).

In some embodiments, R^{Y} is

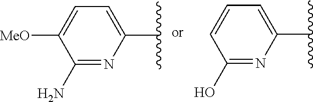 [SMILES: *c1ccc(OC)c(N)n1 ; *c1cccc(O)n1]

(or its tautomer,

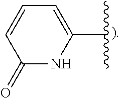 [SMILES: *c1cccc(=O)[nH]1]

In some embodiments, R^{Y} is

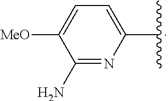 [SMILES: *c1ccc(OC)c(N)n1]

In some embodiments, in R^{Y}, two R² groups on adjacent carbon atoms taken together with the atoms to which they are bound form a fused 4-8 membered ring that is optionally substituted with 1, 2, 3 or 4 groups independently selected from group consisting of R and a nitrogen protecting group, wherein R is as defined in Formula (IV).

In some embodiments, R^{Y} is

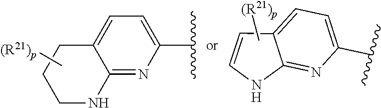

wherein p is 0, 1, 2, 3 or 4; and each R²¹ is independently selected from group consisting of R and a nitrogen protecting group, wherein R is as defined in Formula (IV).

In some embodiments, R^{Y} is

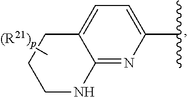

wherein p is 0, 1, 2, 3 or 4; and each R²¹ is independently selected from the group consisting of R and a nitrogen protecting group, wherein R is as defined in Formula (IV).

In some embodiments, R^{Y} is

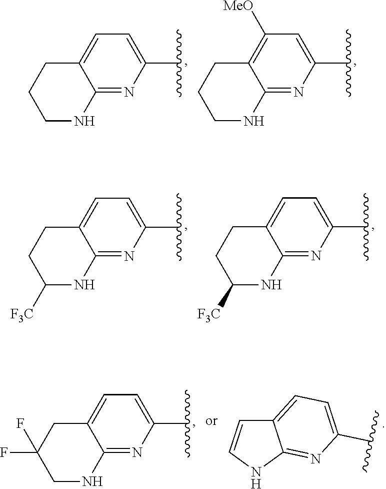

In some embodiments, R^{Y} is

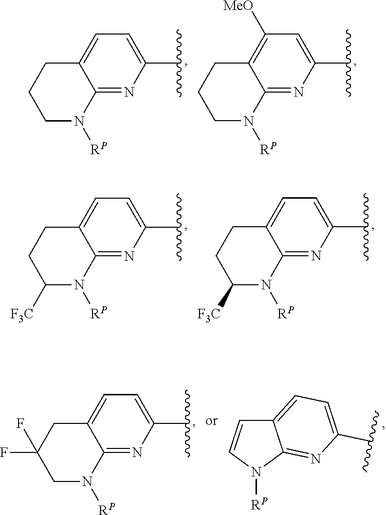

wherein R^{P} is a nitrogen protecting group.

In some embodiments, R^{Y} is

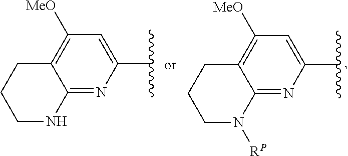

wherein R^{P} is a nitrogen protecting group.

In some embodiments, R^{Y} is

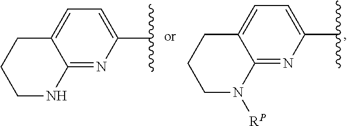

wherein R^{P} is a nitrogen protecting group.

In some embodiments, R^{Y} is

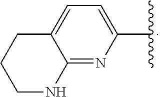 [SMILES: *c1ccc2c(n1)NCCC2]

In some embodiments, R^{Y} is

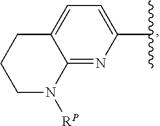

wherein R^{P} is a nitrogen protecting group.

In some embodiments, the compound of Formula (IV) is according to Formula (IV-a) through IV-h):

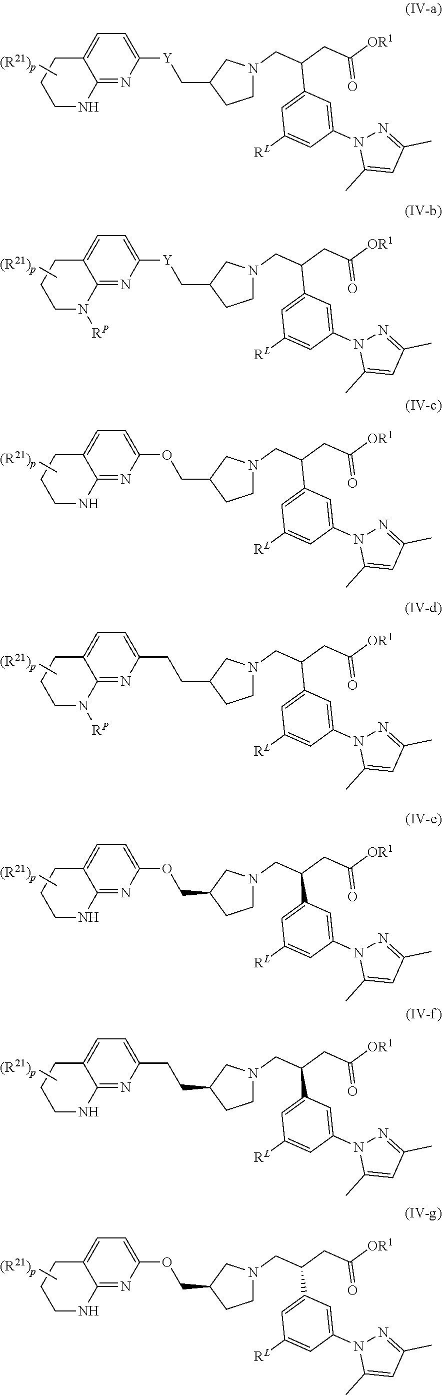

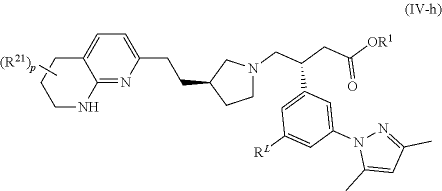

wherein p is 0, 1, 2 or 3;
each R²¹ is independently selected from group consisting of R and a nitrogen protecting group, and R^{P} is a hydrogen or a nitrogen protecting group, and
R is as defined above.
   L Embodiments, Formula (IV) and (IV-a) through (IV-r)

In some embodiments of any one of Formula (IV) and Formulae (IV-a) through (IV-r), L is -L¹-[G-L²]_{q}-G-L³-*, wherein * is the bond to Z.

In another embodiment, wherein L is -L¹-[G-L²]_{q}-G-L³-*, wherein q is 0, 1, 2, 3, 4, or 5.

In another embodiment, L is -L¹-[G-L²]_{q}-G-L³-*, wherein q is 0, 1, 2, 3, or 4

In another embodiment, L is -L¹-[G-L²]_{q}-G-L³-*, wherein q is 0, 1, 2, or 3.

In another embodiment, L is -L¹-[G-L²]_{q}-G-L³-*, wherein q is 0, 1, or 2.

In another embodiment, L is -L¹-[G-L²]_{q}-G-L³-*, wherein q is 1, 2, 3, 4, or 5.

In another embodiment, L is -L¹-[G-L²]_{q}-G-L³-*, wherein q is 1, 2, 3, or 4.

In another embodiment, L is -L¹-[G-L²]_{q}-G-L³-*, wherein q is 1, 2, or 3.

In another embodiment, L is -L¹-[G-L²]_{q}-G-L³-*, wherein q is 1 or 2.

In another embodiment, L is -L¹-[G-L²]_{q}-G-L³-*, wherein q is 4. In another embodiment, L is -L¹-[G-L²]_{q}-G-L³-*, wherein q is 3. In another embodiment, L is -L¹-[G-L²]_{q}-G-L³-*, wherein q is 2.

In another embodiment, L is -L¹-G-L²-G-L³-*. In another embodiment nts, L is -L¹-G-L³-*.

In another embodiment, L is -G-L³-*. In another embodiment, L is -L¹-G-*. In another embodiment, L is -G-*.

In some embodiments, each instance of A-B-A- is independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl.

In some embodiments, each instance of A-B-A- is independently selected from the group consisting of —C(O)O—, —OC(O)—, —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl.

In some embodiments, each instance of A-B-A- is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl.

In some embodiments, each instance of A-B-A- is independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl.

In some embodiments, each instance of A-B-A- is independently selected from the group consisting of —C(O)N(R^{N})—, —N(R^{N})C(O)—, —O—, and —N(R^{N})—, wherein each R^{N} is independently hydrogen or C₁₋₆alkyl.

In some embodiments,
D and F are each independently a bond, C₁₋₁₀alkyl, C₂₋₁₀alkenyl, or C₂₋₁₀alkynyl, each optionally substituted with 1, 2, 3, or 4 R groups; and
E is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments,
D and F are each independently a bond or C₁₋₁₀alkyl optionally substituted with 1, 2, 3, or 4 R groups; and
E is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments, each G is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments, each G is independently C₁₋₁₀alkyl, optionally substituted with 1, 2, or 3 R groups.

In some embodiments, each G is independently C₁₋₁₀alkyl, optionally substituted with 1 or 2 R groups.

In some embodiments, each G is independently C₁₋₁₀alkyl, optionally substituted with one R group

In some embodiments, L is -L1-G-L³-*, wherein * is the bond to Z;
G is -D-E-F-, wherein D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-A-;
L³ is a bond or -A-B-A-;
each A is independently a bond, —O—, —S—, or —N(R^{N})—, wherein each R^{N} is independently hydrogen or C₁₋₆alkyl.
each B is independently a bond, CH₂, C(O), S(O)₂, P(O)(OH), or P(S)(OH); and

In some embodiments, L is -L¹-G-L³-*, wherein * is the bond to Z;
G is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-;
L³ is a bond or -A-B-A-;
each A is independently a bond, —O—, —S—, or —N(R^{N})—, wherein each R^{N} is independently hydrogen or C₁₋₆alkyl; and
each B is independently a bond, CH₂, C(O), S(O)₂, P(O)(OH), or P(S)(OH; and
In some embodiments, L is -L¹-G-*, wherein * is the bond to Z;
G is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-A-, wherein,
   A is a bond, —O—, —S—, or —N(R^{N})—, wherein each R^{N} is independently hydrogen or C₁₋₆ alkyl; and
   B is a bond, C(O), C(S), S(O)₂, P(O)(OH), or P(S)(OH).

In some embodiments, L is -L¹-G-*, wherein * is the bond to Z;
G is C₁₋₁₀alkyl, C₂₋₁₀alkenyl, C₂₋₁₀alkynyl, each of which is optionally substituted with 1 or 2 R groups;
L¹ is -B-A-, wherein
   A is a bond, —O—, —S—, or —N(R^{N})—, each R^{N} is independently hydrogen or C₁₋₆alkyl; and
   B is a bond, C(O), S(O)₂, P(O)(OH), or P(S)(OH).

In some embodiments L is -L¹-G-*, wherein * is the bond to Z;
G is C₁₋₁₀alkyl or C₂₋₁₀alkenyl, each of which is optionally substituted with 1 or 2 R groups;
L¹ is bond, C(O), S(O)₂, P(O)(OH), or P(S)(OH); and
R^{N} is hydrogen or C₁₋₆alkyl.

In some embodiments, L is

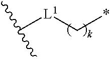

wherein * is the bond to Z; k is an integer from 1 to 10; L¹ is bond, C(O), C(S), C(NR^{N}), S(O)₂, P(O)(OH), or P(S)(OH); and R^{N} is hydrogen or C₁₋₆ alkyl.

In some embodiments, L is

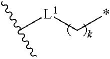

wherein * is the bond to Z; k is an integer from 1 to 10; L¹ is bond, C(O), P(O)(OH), or P(S)(OH).

In some embodiments, L is

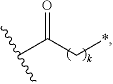

wherein * is the bond to Z; k is an integer from 1 to 10; or an integer from 2 to 10; or an integer from 3 to 10; or an integer from 4 to 10; or an integer from 5 to 10; or an integer from 5 to 9; or an integer from 5 to 8; or an integer from 5 to 7.

In some embodiments, L is

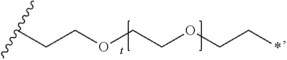

wherein * is the bond to Z; t is an integer from 0 to 10 (e.g., an integer from 1 to 5; or 1; or 2; or 3).

In some embodiments, L is

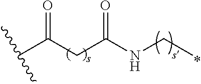

wherein * is the bond to Z, t is an integer from 0 to 10 (e.g., an integer from 1 to 5 or 1; or 2; or 3); a is an integer from 1 to 3; and s and s′ are each independently an integer from 1 to 24 (e.g., an integer from 1 to 16; an integer from 1 to 10; an integer from 3 to 10; an integer from 3 to 7; or an integer from 4 to 6).

In some embodiments, L is

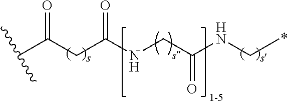

wherein * is the bond to Z; a is 1, 2 or 3; and each s, s′, and s″ independently is an integer from 1 to 24 (e.g., an integer from 1 to 16, an integer from 1 to 10, an integer from 3 to 10, an integer from 3 to 7, or an integer from 4 to 6).

In some embodiments, L is

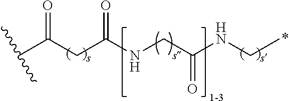

wherein * is the bond to Z; and s, s′, and s″ are independently is an integer from 1 to 24 (e.g., an integer from 1 to 16, an integer from 1 to 10, an integer from 3 to 10, an integer from 3 to 7, or an integer from 4 to 6).

In some embodiments, L is

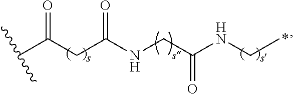

wherein * is the bond to Z and each s, s′, and s″ independently is an integer from 1 to 24 (e.g., an integer from 1 to 16, an integer from 1 to 10, an integer from 3 to 10, an integer from 3 to 7, or an integer from 4 to 6)

In some embodiments, L is

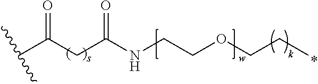

wherein * is the bond to Z; s and k are independently is an integer from 1 to 20 (e.g., an integer from 1 to 16, an integer from 1 to 10, an integer from 3 to 10, an integer from 3 to 7 or an integer from 4 to 6); and w is an integer from 1 to 10 (e.g., an integer from 1 to 16, an integer from 1 to 10, an integer from 3 to 10, an integer from 3 to 7 or an integer from 4 to 6).

In some embodiments, L is

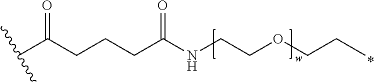

wherein * is the bond to Z and w is an integer from 1 to 20 (e.g., an integer from 1 to 16, an integer from 1 to 10, an integer from 3 to 10, an integer from 3 to 7 or an integer from 4 to 6).
R^{L} Embodiments, Formula (IV) and (IV-a) through (IV-h)

In some embodiments of any one of Formula (IV) and Formulae (IV-a) through (IV-h), R^{L} is —N(R³)(R⁴), wherein R³ is hydrogen or C₁₋₆alkyl and R⁴ is R⁵.

In some embodiments, R^{L} is —N(R³)(R⁴), wherein R³ is hydrogen and R⁴ is R⁵.

In some embodiments, R^{L} is —N(R³)(R⁴), wherein R³ is C₁₋₃alkyl and R⁴ is R⁵.

In some embodiments, R^{L} is —N(R³)(R⁴), wherein R³ is methyl and R⁴ is R⁵.

In some embodiments, R^{L} is —N(R³)(R⁴), wherein R³ and R⁴ taken together with the nitrogen atom to which they are attached form a 4-8 membered monocyclic heterocyclyl group that is substituted with R⁵.

In some embodiments, R^{L} is —N(R³)(R⁴), wherein R³ and R⁴ taken together with the nitrogen atom to which they are attached form a 4-8 membered monocyclic heterocyclyl group that is substituted with R⁵, provided that R³ and R⁴ taken together with the nitrogen atom to which they are attached do not form a morpholino group.

In some embodiments, R^{L} is —N(R³)(R⁴), wherein R³ and R⁴ taken together with the nitrogen atom to which they are attached form a group that is piperidinyl, piperazinyl, pyrrolidinyl, imidazolidinyl, pyrazolidinyl, oxazolidinyl, thiazolidinyl, azetidinyl, pyrrolinyl, imidazolinyl, or pyrazolinyl, each substituted with R⁵.

In some embodiments, R^{L} is

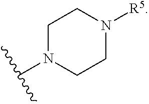

In some embodiments, R^{L} is

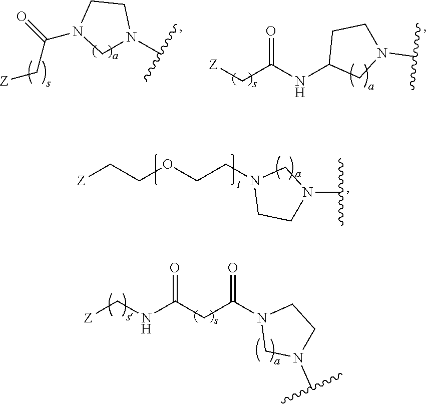

wherein t is an integer from 0 to 10 (e.g., 0, 1, 2, 3, 4, 5, 6, 7, 8, 9 or 10); a is an integer from 1 to 3 and s and s′ are each independently an integer from 1 to 24 (e.g., an integer from 1 to 16, an integer from 1 to 10, an integer from 3 to 10, an integer from 3 to 7 or an integer from 4 to 6).

In some embodiments, R^{L} is

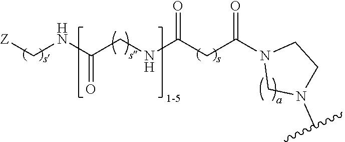

a is an integer from 1 to 3; and s, s′, and s″ are each independently an integer from 1 to 24 (e.g., an integer from 1 to 16, an integer from 1 to 10, an integer from 3 to 10, an integer from 3 to 7 or an integer from 4 to 6).
In some embodiments, R^{L} is

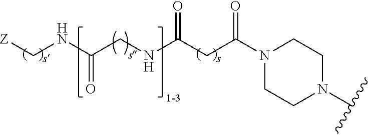

and s, s′, and s″ are independently is an integer from 1 to 24 (e.g., an integer from 1 to 16, an integer from 1 to 10, an integer from 3 to 10, an integer from 3 to 7 or an integer from 4 to 6.

In some embodiments, R^{L} is

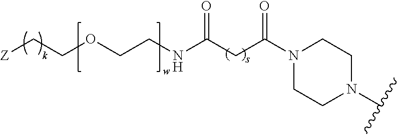

wherein s and k are independently is an integer from 1 to 20 (e.g., an integer from 1 to 16, an integer from 1 to 10, an integer from 3 to 10, an integer from 3 to 7 or an integer from 4 to 6); and w is an integer from 1 to 10 (e.g., an integer from 1 to 16, an integer from 1 to 10, an integer from 3 to 10, an integer from 3 to 7 or an integer from 4 to 6).

In some embodiments, R^{L} is

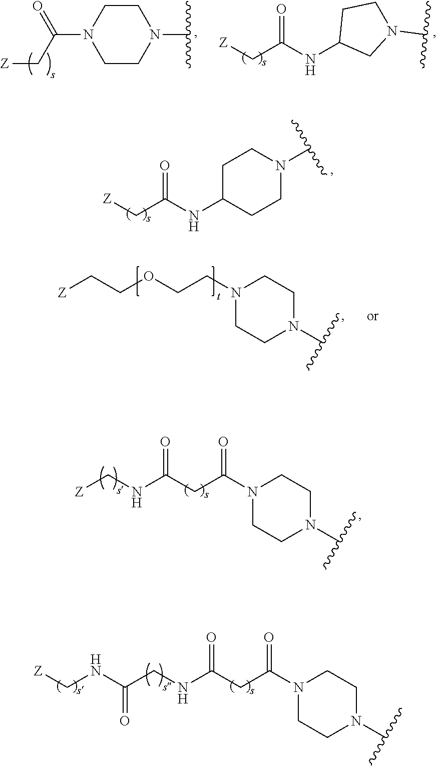

wherein s, s′, and s″ are independently an integer from 1 to 24 (e.g., an integer from 1 to 16, an integer from 1 to 10, an integer from 3 to 10, an integer from 3 to 7 or an integer from 4 to 6); and t is an integer from 1 to 10 (e.g., an integer from 1 to 8, an integer from 1 to 5, an integer from 1 to 3, or 1, or 2, or 3).

In some embodiments, R^{L} is

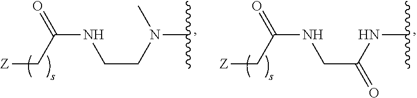

wherein s is an integer from 1 to 24 (e.g., an integer from 1 to 16, an integer from 1 to 10, an integer from 3 to 10, an integer from 3 to 7 or an integer from 4 to 6).

In some embodiments, R^{L} is —O(R⁵).

In some embodiments, R^{L} is

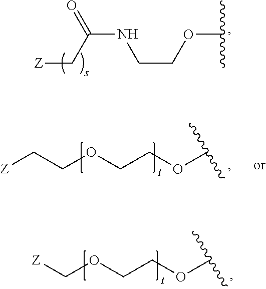

wherein s is an integer from 1 to 24 (e.g., an integer from 1 to 16, an integer from 1 to 10, an integer from 3 to 10, an integer from 3 to 7 or an integer from 4 to 6); and t is an integer from 0 to 10 (e.g., an integer from 1 to 8, an integer from 1 to 5, an integer from 1 to 3, or 1, or 2, or 3).

In some embodiments, R^{L} is —R⁵.

In some embodiments, R^{L} is

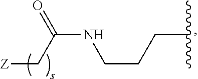

wherein s is 1-24 (e.g., an integer from 1 to 16, an integer from 1 to 10, an integer from 3 to 10, an integer from 3 to 7 or an integer from 4 to 6).

In some embodiments, R^{L} is

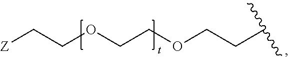

wherein t is 0 to 10 (e.g., 1-5; or 1-3; or 1; or 2; or 3).

In some embodiments, R^{L} is

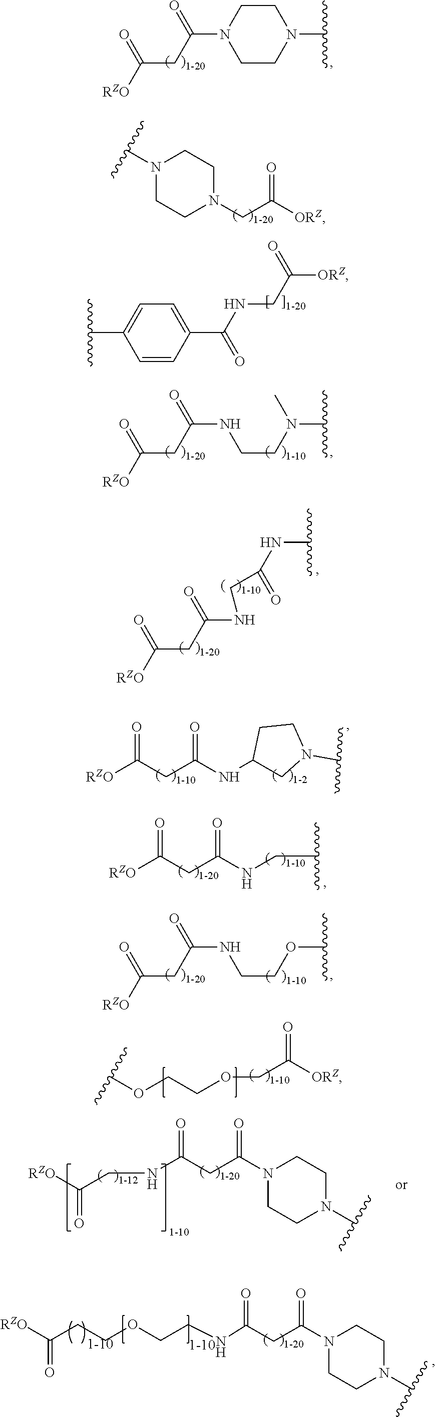

In some embodiments, R^{L} is

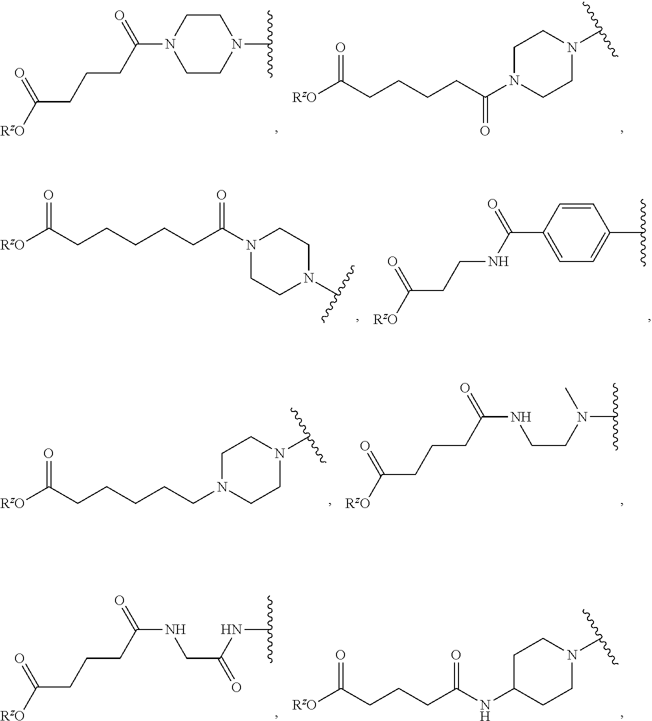

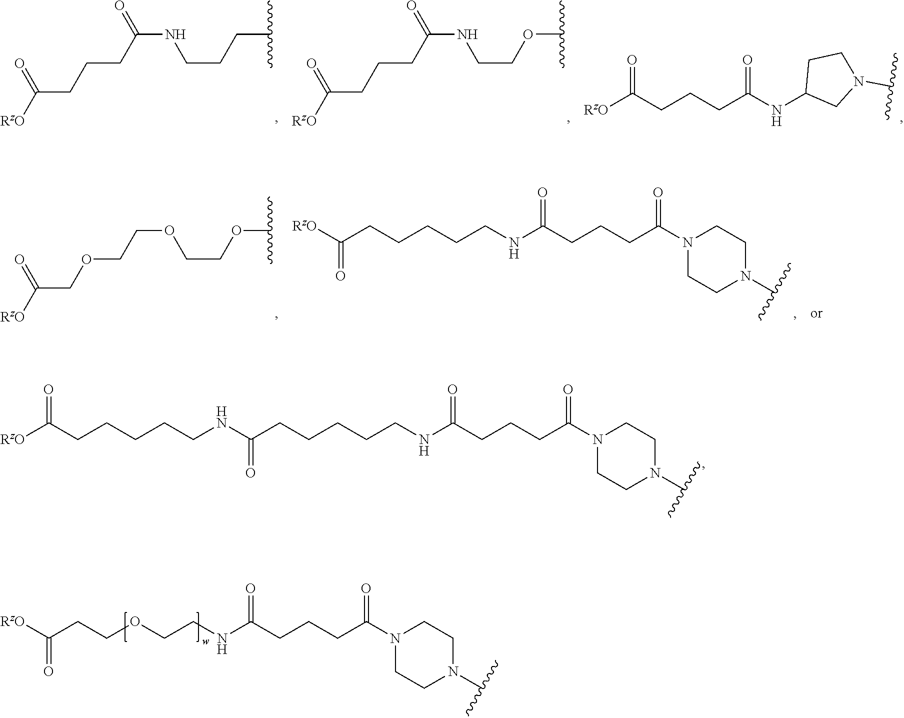

wherein R^{Z} is hydrogen or C₁₋₁₀alkyl, and w is an integer selected from 1-10 (e.g., 2-10, 2-8, 4-8). In some embodiments, R^{Z} is hydrogen or methyl. In some embodiments, R^{Z} is hydrogen. In some embodiments, R^{Z} is methyl.

In some embodiments, R^{L} is

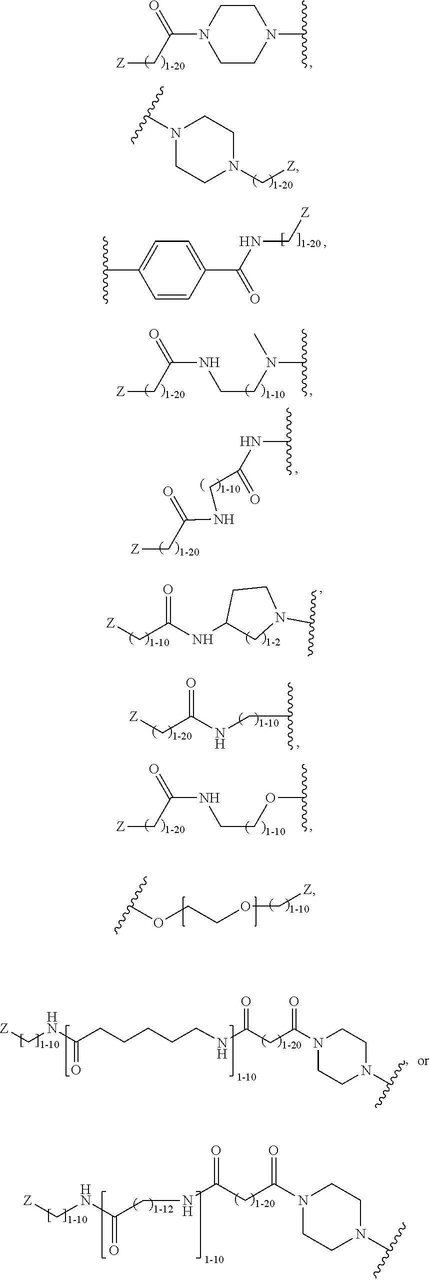

wherein Z is as defined in Formula (IV), and embodiments thereof.

In some embodiments, R^{L} is

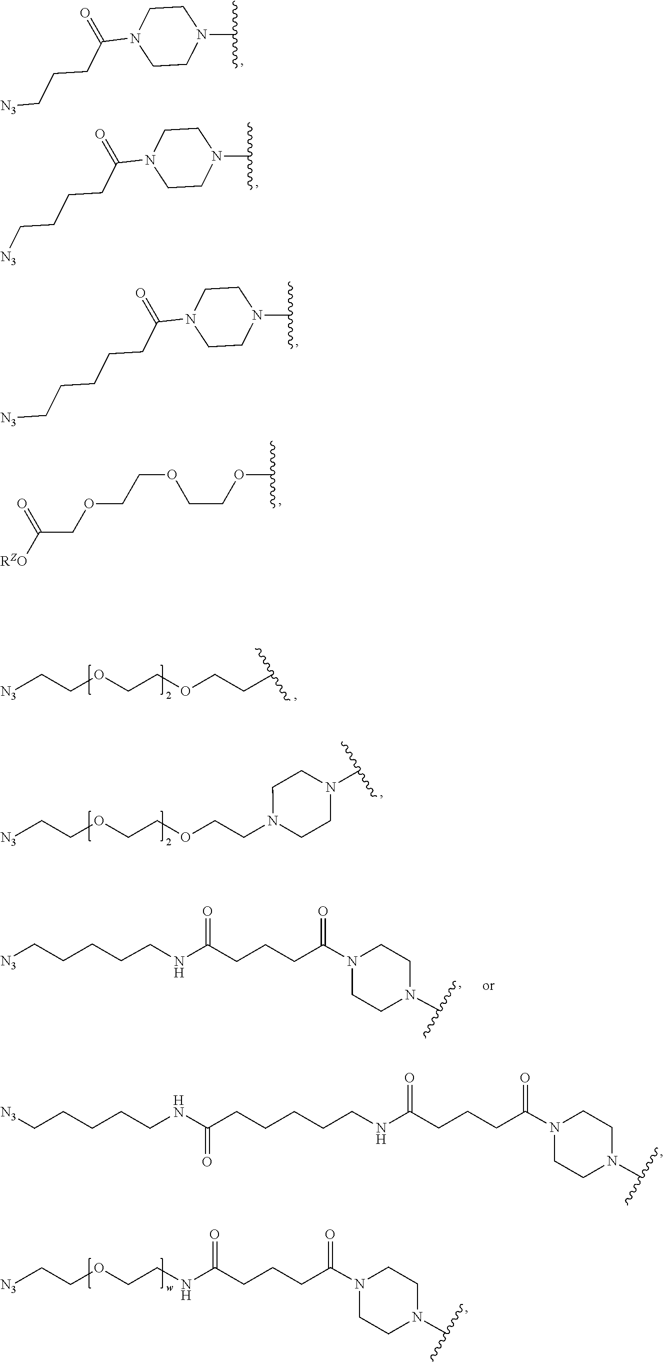

Wherein w is an integer selected from 1-10 (e.g., 2-10, 2-8, 4-8), and R^{Z} is as defined in Formula (IV), and embodiments thereof.

In some embodiments, the compound of Formula (IV) is according to one of Formulae (IV-i) through (IV-1):

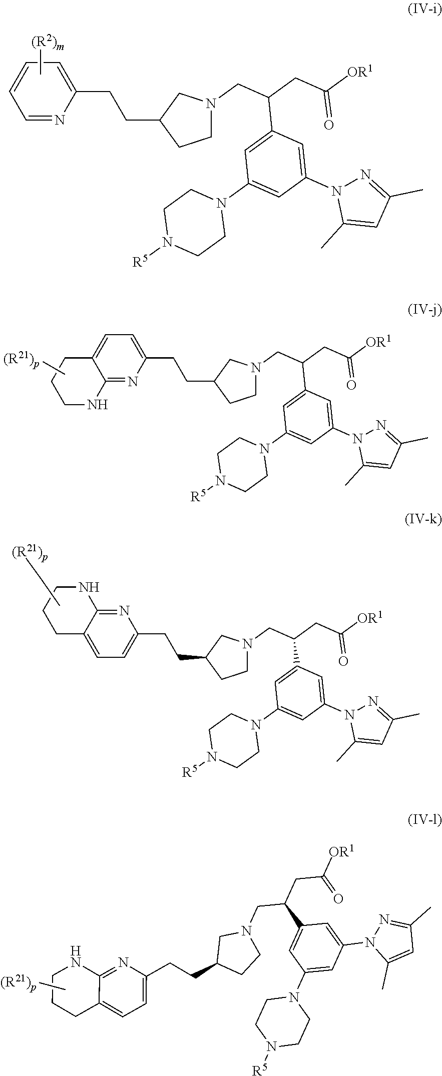

wherein p is 0, 1, 2, or 3 (e.g., 0); each R²¹ is independently selected from group consisting of R and a nitrogen protecting group, wherein R and the remaining variables are as defined in Formula (IV). In one embodiment of Formulae (IV-i) through (IV-1), R¹ is hydrogen. In another embodiment of Formulae (IV-i) through (IV-1), R¹ is C₁₋₆alkyl (e.g., methyl ort-butyl).

In some embodiments, the compound of Formula (IV) is according to one of Formulae (IV-m) through (IV-r):

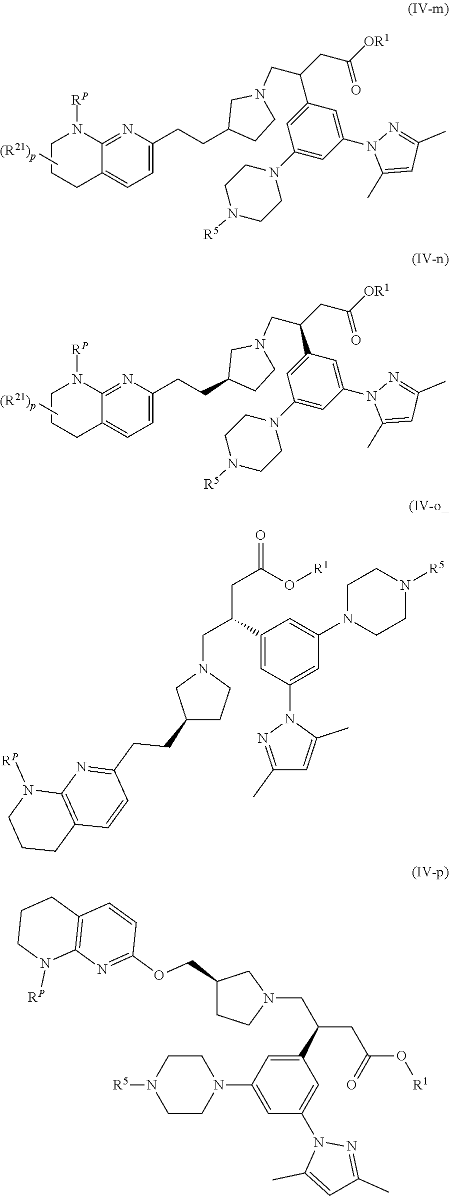

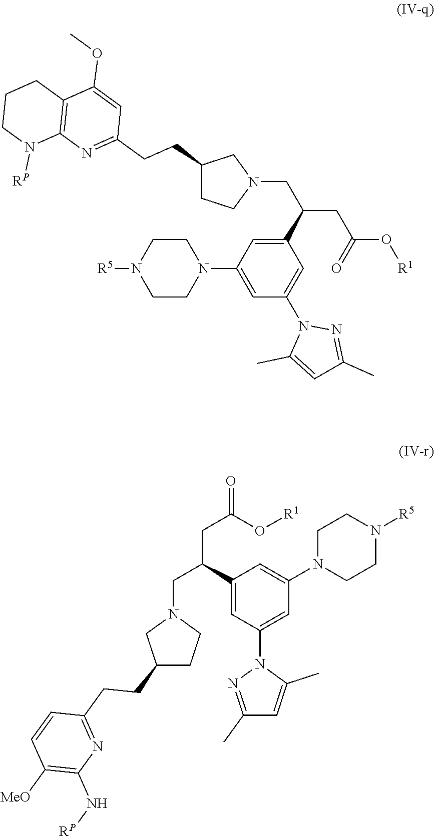

or a salt thereof, wherein p is 0, 1 or 2; each R²¹ is independently selected from group consisting of R; and R^{P} is hydrogen or a nitrogen protecting group (e.g., a nitrogen protecting group), wherein R and the remaining variables are as defined in Formula (IV). In one embodiment of Formulae (IV-m) through (IV-vr), R¹ is hydrogen. In another embodiment of Formulae (IV-m) through (IV-r), R¹ is C₁₋₆alkyl (e.g., methyl or t-butyl).
Z Embodiments, Formulae (IV) and (IV-a) through (IV-r)

In some embodiments of any one of Formula (IV) and Formulae (IV-a) through (IV-r), Z is azido, hydroxy, amino, —N═C═O, —N═C═S, —SR^{Z1}, —C(O)H, —C(O)OR^{Z}, —C(S)OR^{Z}, —CH₂—X, or a Michael acceptor,
wherein R^{Z} is hydrogen or C₁₋₁₀alkyl;
R^{Z1} is hydrogen, pyridyl, or benzotriazolyl;
X is a leaving group.

In some embodiments, Z is COOH.

In some embodiments, Z is NH₂.

In some embodiments, Z is N₃.

In some embodiments, Z is hydroxy.

In some embodiments, Z is —SH.

In some embodiments, Z is

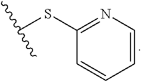 [SMILES: *Sc1ccccn1]

In some embodiments, Z is a Michael acceptor (e.g., N-maleimido).

In some embodiments, Z comprises a terminal alkyne,

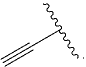 [SMILES: *C#C]

In some embodiments, Z comprises

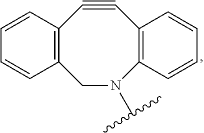 [SMILES: *N1Cc2ccccc2C#Cc2ccccc21]

such as

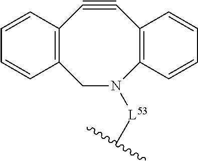

wherein L⁵³ is a bond, —C(O)—, —C(S)—, —S(O)₂—, —C(O)O—, C(O)N(H)—, —S(O)₂N(H)—.

In some embodiments, Z comprises

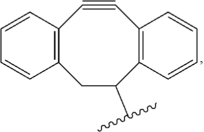 [SMILES: *C1Cc2ccccc2C#Cc2ccccc21]

In some embodiments, Z comprises

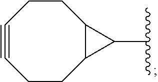 [SMILES: *C1C2CCC#CCCC12]

for example, Z is

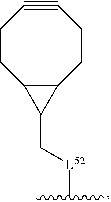

wherein L⁵² is a bond, —O—, —N(H)—, —S—, —C(O)—, —C(S)—, —S(O)₂—, —C(O)O—, —OC(O)—, —C(O)N(H)—, N(H)C(O)—, —OC(O)O—, —OC(O)N(H)—, N(H)C(O)O—, —N(H)C(O)N(H)—, or —S(O)₂N(H)— (e.g.,

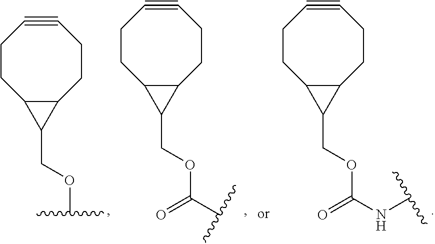

In some embodiments, Z comprises

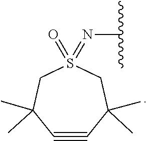

In some embodiments, Z⁰ comprises

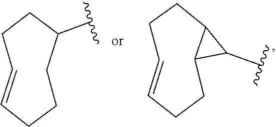 [SMILES: *C1CC/C=C/CCC1 ; *C1C2CC/C=C/CCC12]

such as

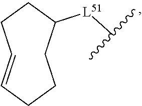

wherein L⁵¹ is a bond, —O—, —N(H)—, —S—, —C(O)—, —C(S)—, —S(O)₂—, —C(O)O—, —OC(O)—, —C(O)N(H)—, N(H)C(O)—, —OC(O)O—, —OC(O)N(H)—, N(H)C(O)O—, —N(H)C(O)N(H)—, —CH₂O—, —CH₂N(H)—, —CH₂S—, —CH₂C(O)O—, —CH₂OC(O)—, —CH₂C(O)N(H)—, —CH₂N(H)C(O)—, —CH₂OC(O)O—, —CH₂OC(O)N(H)—, —CH₂N(H)C(O)O—, —CH₂N(H)C(O)N(H)—, or —S(O)₂N(H)—

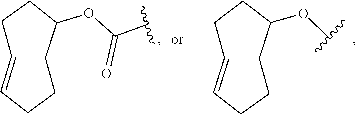 [SMILES: *C(=O)OC1CC/C=C/CCC1 ; *OC1CC/C=C/CCC1]

In some embodiments, Z comprises

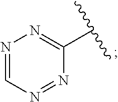 [SMILES: *c1nncnn1]

for example, Z is R^{Z} is

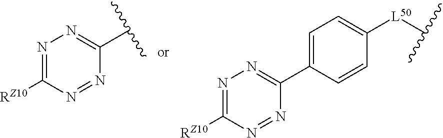

wherein R^{Z10} is hydrogen or C₁₋₁₀alkyl; and L⁵⁰ is a bond, —O—, —N(H)—, —S—, —C(O)—, —C(S)—, —S(O)₂—, —C(O)O—, —OC(O)—, —C(O)N(H)—, N(H)C(O)—, —OC(O)O—, —OC(O)N(H)—, N(H)C(O)O—, —N(H)C(O)N(H)—, —CH₂O—, —CH₂N(H)—, —CH₂S—, —CH₂C(O)O—, —CH₂OC(O)—, —CH₂C(O)N(H)—, —CH₂N(H)C(O)—, —CH₂OC(O)O—, —CH₂OC(O)N(H)—, —CH₂N(H)C(O)O—, —CH₂N(H)C(O)N(H)—, or —S(O)₂N(H)—.

In some embodiments, Z is an activated ester (e.g.,

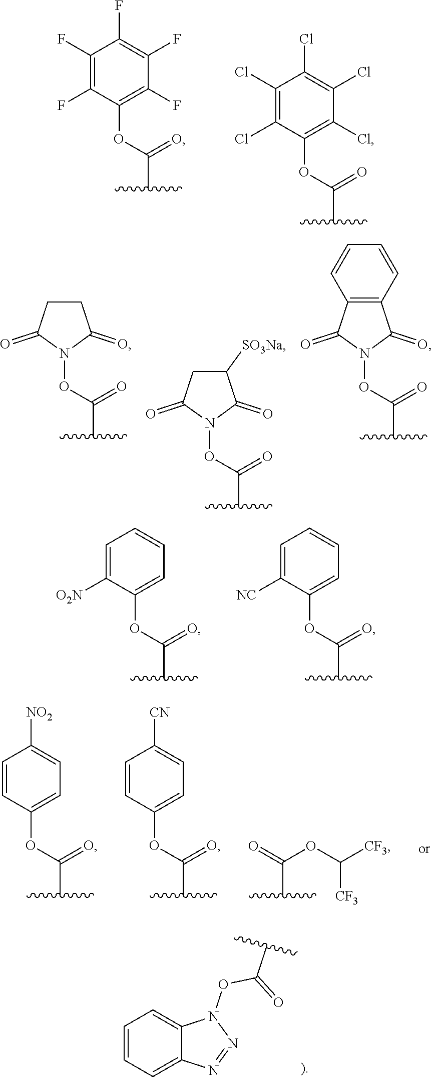

In some embodiments, Z is

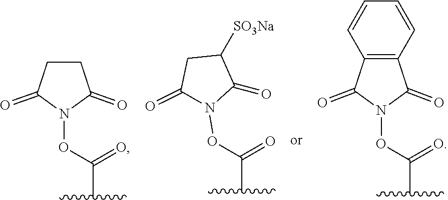

In some embodiments, Z is

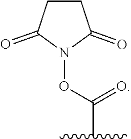 [SMILES: *C(=O)ON1C(=O)CCC1=O]

In some embodiments, Z is

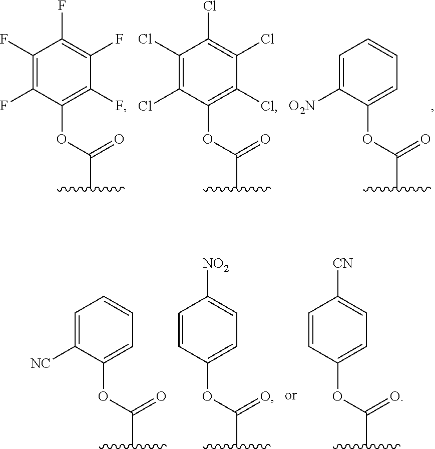

In some embodiments, Z is

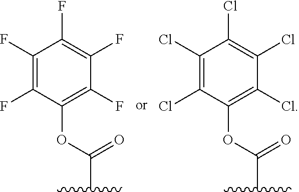 [SMILES: *C(=O)Oc1c(F)c(F)c(F)c(F)c1F ; "or" ; *C(=O)Oc1c(Cl)c(Cl)c(Cl)c(Cl)c1Cl]

In some embodiments, Z is

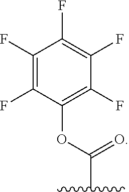 [SMILES: *C(=O)Oc1c(F)c(F)c(F)c(F)c1F]

R^{P} Embodiments, Formula (IV) and (IV-a) through (IV-r)

In some embodiments of any one of Formula (IV) and Formulae (IV-a) through (IV-r), R^{P} is

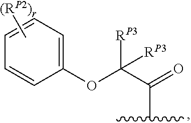

wherein r is 1, 2, or 3; each R² is independently halogen, nitro, cyano, C₁₋₄ alkoxy, C₁₋₄alkyl, C₁₋₄haloalkyl; and each R^{P3} is independently hydrogen, methyl, or ethyl.

In some embodiments, when present, R^{P} is methoxyacetyl (mac), phenoxyacetyl (pac), 2-chlorophenoxyacetyl, 3-chlorophenoxyacetyl, 4-chlorophenoxyacetyl, 2,4-dichlorophenoxyacetyl, 2-methylphenoxyacetyl, 3-methylphenoxyacetyl, 4-methylphenoxyacetyl, 4-chloro-2-methylphenoxyacetyl, 2-nitrophenoxyacetyl, 3-nitrophenoxyacetyl, 4-nitrophenoxyacetyl, 2-isopropylphenoxyacetyl, 3-isopropylphenoxyacetyl, 4-isopropylphenoxyacetyl, 2-(t-butyl)phenoxyacetyl, 3-(t-butyl)phenoxyacetyl, 4-(t-butyl)phenoxyacetyl, 2-fluorophenoxyacetyl, 3-fluorophenoxyacetyl, 4-fluorophenoxyacetyl, 2,4-difluorophenoxyacetyl, 4-(trifluoromethoxy)phenoxyacetyl, 2-phenoxypropanoyl, 2-(4-chloro-2-methylphenoxy)propanoyl, or 2-(4-chlorophenoxy)propanoyl.

In some embodiments, when present, R^{P} is methoxyacetyl (mac), phenoxyacetyl (pac), 2-chlorophenoxyacetyl, 4-chlorophenoxyacetyl, 2-methylphenoxyacetyl, 4-methylphenoxyacetyl, or 4-isopropylphenoxyacetyl.

In some embodiments, when present, R^{P} is methoxyacetyl (mac).

In some embodiments, when present, R^{P} is phenoxyacetyl (pac).

In some embodiments, when present, R¹ is C₁₋₆alkyl and R^{P} is

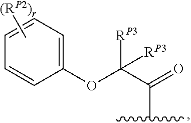

wherein r is 1, 2, or 3; each R^{P2} is independently halogen, nitro, cyano, C₁₋₄alkoxy, C₁₋₄alkyl, C₁₋₄haloalkyl; and each R^{P3} is independently hydrogen, methyl, or ethyl.

In some embodiments, when present, R¹ is C₁₋₆alkyl and R^{P} is methoxyacetyl (mac), phenoxyacetyl (pac), 2-chlorophenoxyacetyl, 3-chlorophenoxyacetyl, 4-chlorophenoxyacetyl, 2,4-dichlorophenoxyacetyl, 2-methylphenoxyacetyl, 3-methylphenoxyacetyl, 4-methylphenoxyacetyl, 4-chloro-2-methylphenoxyacetyl, 2-nitrophenoxyacetyl, 3-nitrophenoxyacetyl, 4-nitrophenoxyacetyl, 2-isopropylphenoxyacetyl, 3-isopropylphenoxyacetyl, 4-isopropylphenoxyacetyl, 2-(t-butyl)phenoxyacetyl, 3-(t-butyl)phenoxyacetyl, 4-(t-butyl)phenoxyacetyl, 2-fluorophenoxyacetyl, 3-fluorophenoxyacetyl, 4-fluorophenoxyacetyl, 2,4-difluorophenoxyacetyl, 4-(trifluoromethoxy)phenoxyacetyl, 2-phenoxypropanoyl, 2-(4-chloro-2-methylphenoxy)propanoyl, or 2-(4-chlorophenoxy)propanoyl.

In some embodiments, when present, R¹ is C₁₋₆alkyl and R^{P} is methoxyacetyl (mac), phenoxyacetyl (pac), 2-chlorophenoxyacetyl, 4-chlorophenoxyacetyl, 2-methylphenoxyacetyl, 4-methylphenoxyacetyl, or 4-isopropylphenoxyacetyl.

In some embodiments, when present, R¹ is C₁₋₆alkyl and R^{P} is methoxyacetyl (mac).

In some embodiments, when present, R¹ is C₁₋₆alkyl and R^{P} is phenoxyacetyl (pac).

In some embodiments, when present, R¹ is methyl and R^{P} is

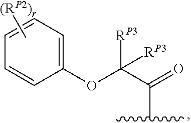

wherein r is 1, 2, or 3; each R^{P2} is independently halogen, nitro, cyano, C₁₋₄alkoxy, C₁₋₄alkyl, C₁₋₄haloalkyl; and each R^{P3} is independently hydrogen, methyl, or ethyl.

In some embodiments, when present, R¹ is methyl and R^{P} is methoxyacetyl (mac), phenoxyacetyl (pac), 2-chlorophenoxyacetyl, 3-chlorophenoxyacetyl, 4-chlorophenoxyacetyl, 2,4-dichlorophenoxyacetyl, 2-methylphenoxyacetyl, 3-methylphenoxyacetyl, 4-methylphenoxyacetyl, 4-chloro-2-methylphenoxyacetyl, 2-nitrophenoxyacetyl, 3-nitrophenoxyacetyl, 4-nitrophenoxyacetyl, 2-isopropylphenoxyacetyl, 3-isopropylphenoxyacetyl, 4-isopropylphenoxyacetyl, 2-(t-butyl)phenoxyacetyl, 3-(t-butyl)phenoxyacetyl, 4-(t-butyl)phenoxyacetyl, 2-fluorophenoxyacetyl, 3-fluorophenoxyacetyl, 4-fluorophenoxyacetyl, 2,4-difluorophenoxyacetyl, 4-(trifluoromethoxy)phenoxyacetyl, 2-phenoxypropanoyl, 2-(4-chloro-2-methylphenoxy)propanoyl, or 2-(4-chlorophenoxy)propanoyl.

In some embodiments, when present, R¹ is methyl and R^{P} is methoxyacetyl (mac), phenoxyacetyl (pac), 2-chlorophenoxyacetyl, 4-chlorophenoxyacetyl, 2-methylphenoxyacetyl, 4-methylphenoxyacetyl, or 4-isopropylphenoxyacetyl.

In some embodiments, when present, R¹ is methyl and R^{P} is methoxyacetyl (mac).

In some embodiments, when present, R¹ is methyl and R^{P} is phenoxyacetyl (pac).

In some embodiments, when present, R¹ is hydrogen and R^{P} is

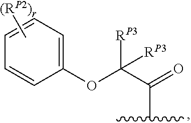

wherein r is 1, 2, or 3; each R^{P2} is independently halogen, nitro, cyano, C₁₋₄alkoxy, C₁₋₄alkyl, C₁₋₄haloalkyl; and each R^{P3} is independently hydrogen, methyl, or ethyl.

In some embodiments, when present, R¹ is hydrogen and R^{P} is methoxyacetyl (mac), phenoxyacetyl (pac), 2-chlorophenoxyacetyl, 3-chlorophenoxyacetyl, 4-chlorophenoxyacetyl, 2,4-dichlorophenoxyacetyl, 2-methylphenoxyacetyl, 3-methylphenoxyacetyl, 4-methylphenoxyacetyl, 4-chloro-2-methylphenoxyacetyl, 2-nitrophenoxyacetyl, 3-nitrophenoxyacetyl, 4-nitrophenoxyacetyl, 2-isopropylphenoxyacetyl, 3-isopropylphenoxyacetyl, 4-isopropylphenoxyacetyl, 2-(t-butyl)phenoxyacetyl, 3-(t-butyl)phenoxyacetyl, 4-(t-butyl)phenoxyacetyl, 2-fluorophenoxyacetyl, 3-fluorophenoxyacetyl, 4-fluorophenoxyacetyl, 2,4-difluorophenoxyacetyl, 4-(trifluoromethoxy)phenoxyacetyl, 2-phenoxypropanoyl, 2-(4-chloro-2-methylphenoxy)propanoyl, or 2-(4-chlorophenoxy)propanoyl.

In some embodiments, when present, R¹ is hydrogen and R^{P} is methoxyacetyl (mac), phenoxyacetyl (pac), 2-chlorophenoxyacetyl, 4-chlorophenoxyacetyl, 2-methylphenoxyacetyl, 4-methylphenoxyacetyl, or 4-isopropylphenoxyacetyl.

In some embodiments, when present, R¹ is hydrogen and R^{P} is methoxyacetyl (mac).

In some embodiments, when present, R¹ is hydrogen and R^{P} is phenoxyacetyl (pac).

### Species Embodiments, Formula (IV)

In some embodiments, the compound of Formula (IV) is selected from the group consisting of,

| | |
|---|---|
| 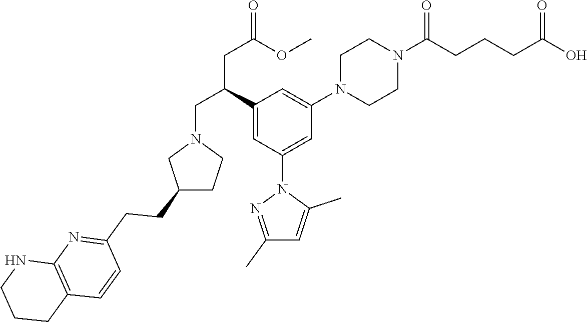 [SMILES: COC(=O)C[C@H](CN1CC[C@@H](CCc2ccc3c(n2)NCCC3)C1)c1cc(N2CCN(C(=O)CCCC(=O)O)CC2)cc(-n2nc(C)cc2C)c1] | 5-(4-(3-(3,5-dimethyl-1H-pyrazol-1-yl)-5-((S)-4-methoxy- 4-oxo-1-((R)-3-(2-(5,6,7,8-tetrahydro-1,8-naphthyridin-2- yl)ethyl)pyrrolidin-1-yl)butan-2-yl)phenyl)piperazin-1- yl)-5-oxopentanoic acid; |
| 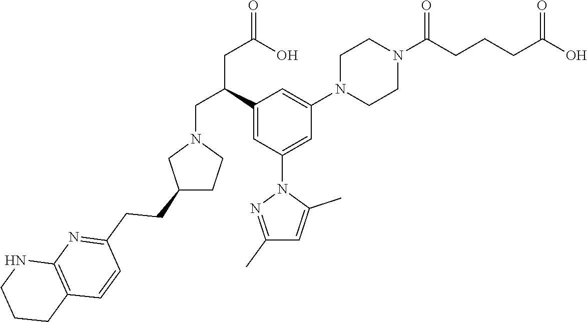 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)O)CC3)c2)n1] | 5-(4-(3-((S)-1-carboxy-3-((R)-3-(2-(5,6,7,8-tetrahydro- 1,8-naphthyridin-2-yl)ethyl)pyrrolidin-1-yl)propan-2-yl)- 5-(3,5-dimethyl-1H-pyrazol-1-yl)phenyl)piperazin-1-yl)- 5-oxopentanoic acid; |
| 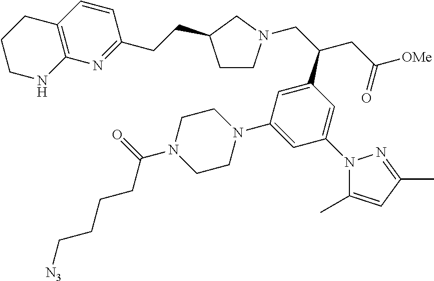 [SMILES: COC(=O)C[C@H](CN1CC[C@@H](CCc2ccc3c(n2)NCCC3)C1)c1cc(N2CCN(C(=O)CCCCN=[N+]=[N-])CC2)cc(-n2nc(C)cc2C)c1] | methyl (S)-3-(3-(4-(5-azidopentanoyl)piperazin-1-yl)-5- (3,5-dimethyl-1H-pyrazol-1-yl)phenyl)-4-((R)-3-(2- (5,6,7,8-tetrahydro-1,8-naphthyridin-2-yl)ethyl)pyrrolidin- 1-yl)butanoate; |
| 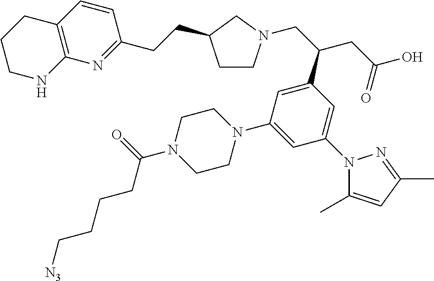 [SMILES: Cc1cc(C)n(-c2cc([C@H](CC(=O)O)CN3CC[C@@H](CCc4ccc5c(n4)NCCC5)C3)cc(N3CCN(C(=O)CCCCN=[N+]=[N-])CC3)c2)n1] | (S)-3-(3-(4-(5-azidopentanoyl)piperazin-1-yl)-5-(3,5- dimethyl-1H-pyrazol-1-yl)phenyl)-4-((R)-3-(2-(5,6,7,8- tetrahydro-1,8-naphthyridin-2-yl)ethyl)pyrrolidin-1- yl)butanoic acid; |
| 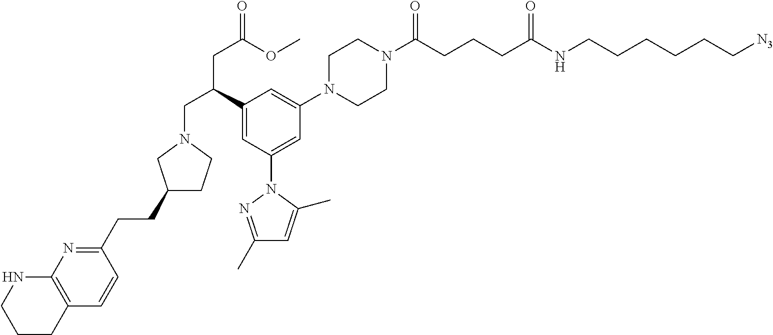 [SMILES: COC(=O)C[C@H](CN1CC[C@@H](CCc2ccc3c(n2)NCCC3)C1)c1cc(N2CCN(C(=O)CCCC(=O)NCCCCCCN=[N+]=[N-])CC2)cc(-n2nc(C)cc2C)c1] | methyl (S)-3-(3-(4-(5-((6-azidohexyl)amino)-5- oxopentanoyl)piperazin-1-yl)-5-(3,5-dimethyl-1H- pyrazol-1-yl)phenyl)-4-((R)-3-(2-(5,6,7,8-tetrahydro-1,8- naphthyridin-2-yl)ethyl)pyrrolidin-1-yl)butanoate; |
| 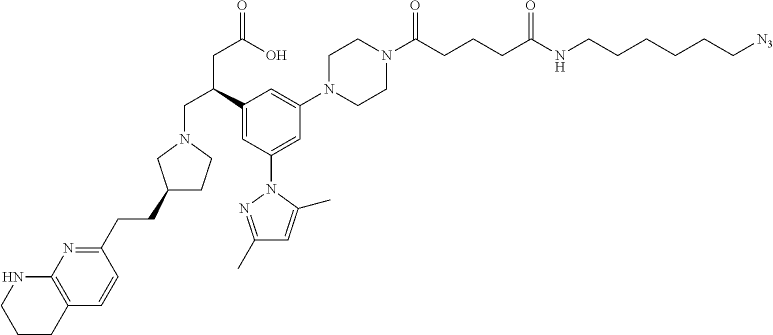 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCCCCN=[N+]=[N-])CC3)c2)n1] | (S)-3-(3-(4-(5-((6-azidohexyl)amino)-5- oxopentanoyl)piperazin-1-yl)-5-(3,5-dimethyl-1H- pyrazol-1-yl)phenyl)-4-((R)-3-(2-(5,6,7,8-tetrahydro-1,8- naphthyridin-2-yl)ethyl)pyrrolidin-1-yl)butanoic acid; |
| 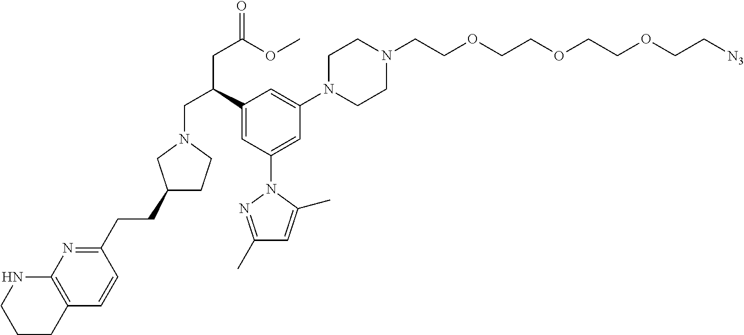 [SMILES: COC(=O)C[C@H](CN1CC[C@@H](CCc2ccc3c(n2)NCCC3)C1)c1cc(N2CCN(CCOCCOCCOCCN=[N+]=[N-])CC2)cc(-n2nc(C)cc2C)c1] | methyl (S)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-((R)-3-(2-(5,6,7,8- tetrahydro-1,8-naphthyridin-2-yl)ethyl)pyrrolidin-1- yl)butanoate |
| 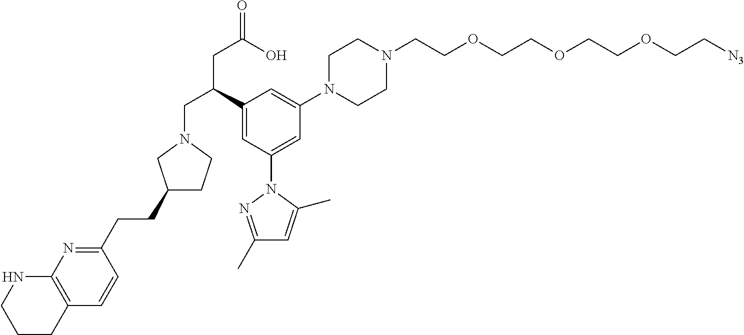 [SMILES: Cc1cc(C)n(-c2cc([C@H](CC(=O)O)CN3CC[C@@H](CCc4ccc5c(n4)NCCC5)C3)cc(N3CCN(CCOCCOCCOCCN=[N+]=[N-])CC3)c2)n1] | (S)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-((R)-3-(2-(5,6,7,8- tetrahydro-1,8-naphthyridin-2-yl)ethyl)pyrrolidin-1- yl)butanoic acid |
| 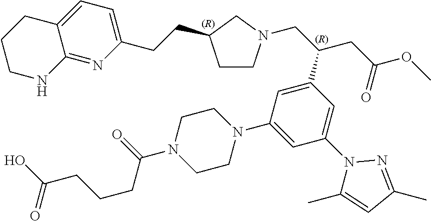 | 5-(4-(3-(3,5-dimethyl-1H-pyrazol-1-yl)-5-((R)-4- methoxy-4-oxo-1-((R)-3-(2-(5,6,7,8-tetrahydro-1,8- naphthyridin-2-yl)ethyl)pyrrolidin-1-yl)butan-2- yl)phenyl)piperazin-1-yl)-5-oxopentanoic acid; |
| 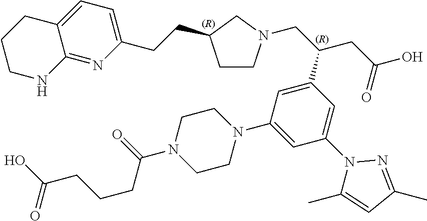 | 5-(4-(3-((R)-1-carboxy-3-((R)-3-(2-(5,6,7,8-tetrahydro- 1,8-naphthyridin-2-yl)ethyl)pyrrolidin-1-yl)propan-2-yl)- 5-(3,5-dimethyl-1H-pyrazol-1-yl)phenyl)piperazin-1-yl)- 5-oxopentanoic acid; |
| 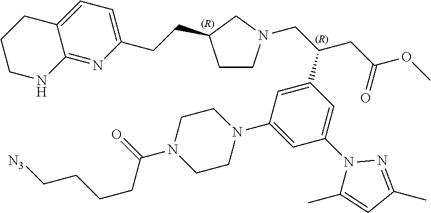 | methyl (R)-3-(3-(4-(5-azidopentanoyl)piperazin-1-yl)-5- (3,5-dimethyl-1H-pyrazol-1-yl)phenyl)-4-((R)-3-(2- (5,6,7,8-tetrahydro-1,8-naphthyridin-2-yl)ethyl)pyrrolidin- 1-yl)butanoate; |
| 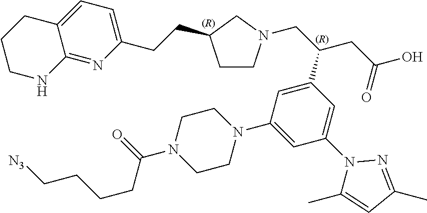 | (R)-3-(3-(4-(5-azidopentanoyl)piperazin-1-yl)-5-(3,5- dimethyl-1H-pyrazol-1-yl)phenyl)-4-((R)-3-(2-(5,6,7,8- tetrahydro-1,8-naphthyridin-2-yl)ethyl)pyrrolidin-1- yl)butanoic acid; |
| 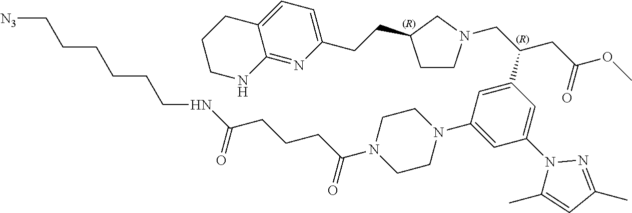 | methyl (R)-3-(3-(4-(5-((6-azidohexyl)amino)-5- oxopentanoyl)piperazin-1-yl)-5-(3,5-dimethyl-1H- pyrazol-1-yl)phenyl)-4-((R)-3-(2-(5,6,7,8-tetrahydro-1,8- naphthyridin-2-yl)ethyl)pyrrolidin-1-yl)butanoate; |
| 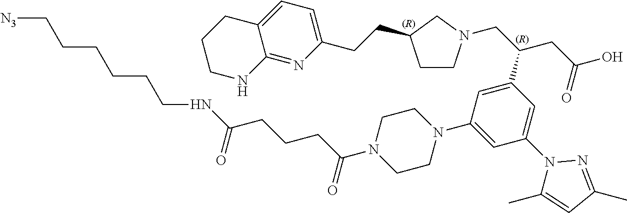 | (R)-3-(3-(4-(5-((6-azidohexyl)amino)-5- oxopentanoyl)piperazin-1-yl)-5-(3,5-dimethyl-1H- pyrazol-1-yl)phenyl)-4-((R)-3-(2-(5,6,7,8-tetrahydro-1,8- naphthyridin-2-yl)ethyl)pyrrolidin-1-yl)butanoic acid; |
| 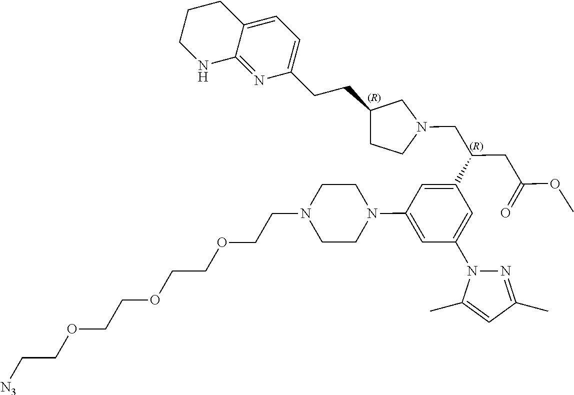 | methyl (R)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-((R)-3-(2-(5,6,7,8- tetrahydro-1,8-naphthyridin-2-yl)ethyl)pyrrolidin-1- yl)butanoate |
| 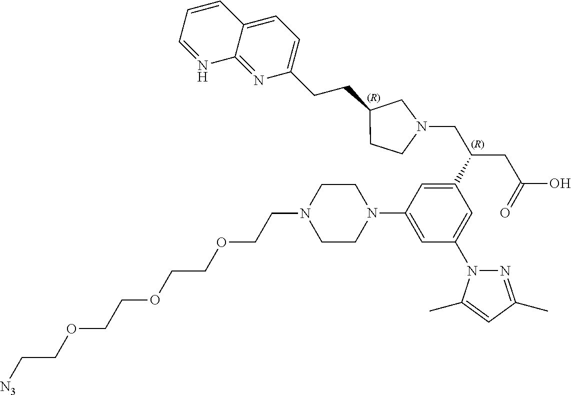 | (R)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-((R)-3-(2-(5,6,7,8- tetrahydro-1,8-naphthyridin-2-yl)ethyl)pyrrolidin-1- yl)butanoic acid |
| 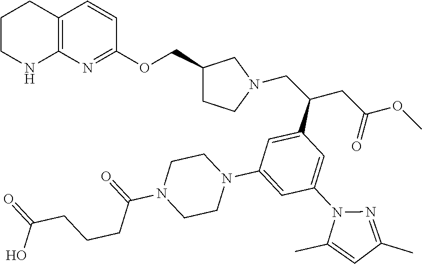 [SMILES: COC(=O)C[C@H](CN1CC[C@@H](COc2ccc3c(n2)NCCC3)C1)c1cc(N2CCN(C(=O)CCCC(=O)O)CC2)cc(-n2nc(C)cc2C)c1] | 5-(4-(3-(3,5-dimethyl-1H-pyrazol-1-yl)-5-((S)-4-methoxy- 4-oxo-1-((R)-3-(((5,6,7,8-tetrahydro-1,8-naphthyridin-2- yl)oxy)methyl)pyrrolidin-1-yl)butan-2- yl)phenyl)piperazin-1-yl)-5-oxopentanoic acid; |
| 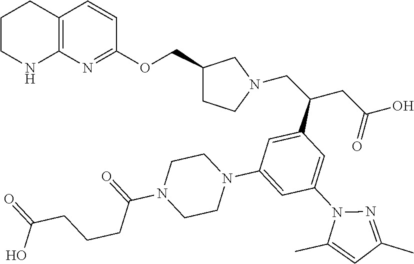 [SMILES: Cc1cc(C)n(-c2cc([C@H](CC(=O)O)CN3CC[C@@H](COc4ccc5c(n4)NCCC5)C3)cc(N3CCN(C(=O)CCCC(=O)O)CC3)c2)n1] | 5-(4-(3-((S)-1-carboxy-3-((R)-3-(((5,6,7,8-tetrahydro-1,8- naphthyridin-2-yl)oxy)methyl)pyrrolidin-1-yl)propan-2- yl)-5-(3,5-dimethyl-1H-pyrazol-1-yl)phenyl)piperazin-1- yl)-5-oxopentanoic acid; |
| 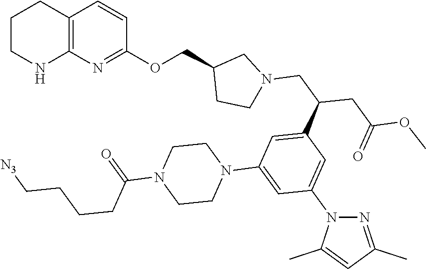 [SMILES: COC(=O)C[C@H](CN1CC[C@@H](COc2ccc3c(n2)NCCC3)C1)c1cc(N2CCN(C(=O)CCCCN=[N+]=[N-])CC2)cc(-n2nc(C)cc2C)c1] | methyl (S)-3-(3-(4-(5-azidopentanoyl)piperazin-1-yl)-5- (3,5-dimethyl-1H-pyrazol-1-yl)phenyl)-4-((R)-3- (((5,6,7,8-tetrahydro-1,8-naphthyridin-2- yl)oxy)methyl)pyrrolidin-1-yl)butanoate; |
| 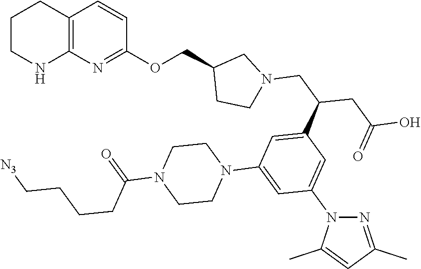 [SMILES: Cc1cc(C)n(-c2cc([C@H](CC(=O)O)CN3CC[C@@H](COc4ccc5c(n4)NCCC5)C3)cc(N3CCN(C(=O)CCCCN=[N+]=[N-])CC3)c2)n1] | (S)-3-(3-(4-(5-azidopentanoyl)piperazin-1-yl)-5-(3,5- dimethyl-1H-pyrazol-1-yl)phenyl)-4-((R)-3-(((5,6,7,8- tetrahydro-1,8-naphthyridin-2-yl)oxy)methyl)pyrrolidin-1- yl)butanoic acid; |
| 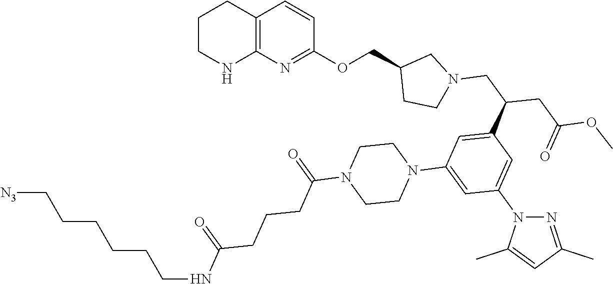 [SMILES: COC(=O)C[C@H](CN1CC[C@@H](COc2ccc3c(n2)NCCC3)C1)c1cc(N2CCN(C(=O)CCCC(=O)NCCCCCCN=[N+]=[N-])CC2)cc(-n2nc(C)cc2C)c1] | methyl (S)-3-(3-(4-(5-((6-azidohexyl)amino)-5- oxopentanoyl)piperazin-1-yl)-5-(3,5-dimethyl-1H- pyrazol-1-yl)phenyl)-4-((R)-3-(((5,6,7,8-tetrahydro-1,8- naphthyridin-2-yl)oxy)methyl)pyrrolidin-1-yl)butanoate; |
| 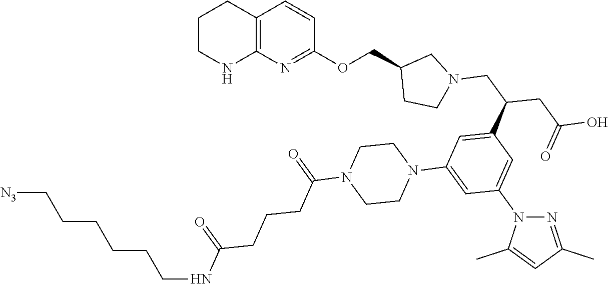 [SMILES: Cc1cc(C)n(-c2cc([C@H](CC(=O)O)CN3CC[C@@H](COc4ccc5c(n4)NCCC5)C3)cc(N3CCN(C(=O)CCCC(=O)NCCCCCCN=[N+]=[N-])CC3)c2)n1] | (S)-3-(3-(4-(5-((6-azidohexyl)amino)-5- oxopentanoyl)piperazin-1-yl)-5-(3,5-dimethyl-1H- pyrazol-1-yl)phenyl)-4-((R)-3-(((5,6,7,8-tetrahydro-1,8- naphthyridin-2-yl)oxy)methyl)pyrrolidin-1-yl)butanoic acid; |
| 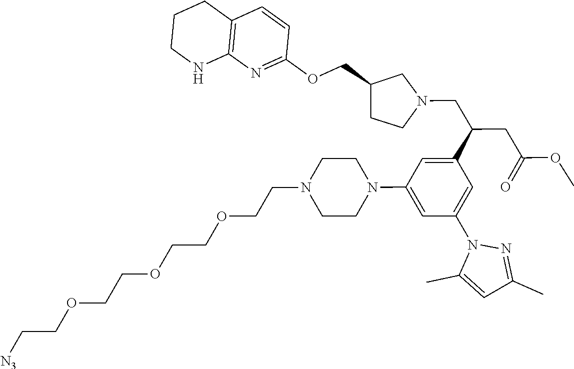 [SMILES: COC(=O)C[C@H](CN1CC[C@@H](COc2ccc3c(n2)NCCC3)C1)c1cc(N2CCN(CCOCCOCCOCCN=[N+]=[N-])CC2)cc(-n2nc(C)cc2C)c1] | methyl (S)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-((R)-3-(((5,6,7,8- tetrahydro-1,8-naphthyridin-2-yl)oxy)methyl)pyrrolidin-1- yl)butanoate |
| 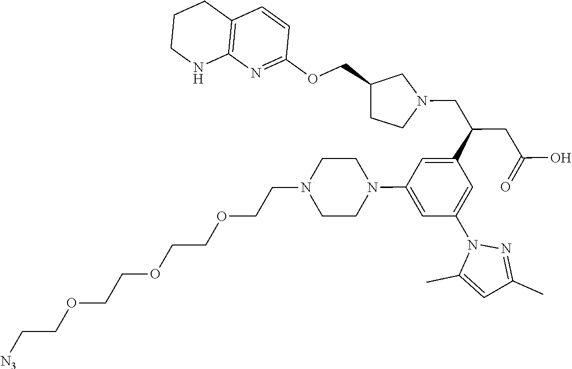 [SMILES: Cc1cc(C)n(-c2cc([C@H](CC(=O)O)CN3CC[C@@H](COc4ccc5c(n4)NCCC5)C3)cc(N3CCN(CCOCCOCCOCCN=[N+]=[N-])CC3)c2)n1] | (S)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-((R)-3-(((5,6,7,8- tetrahydro-1,8-naphthyridin-2-yl)oxy)methyl)pyrrolidin-1- yl)butanoic acid |
| 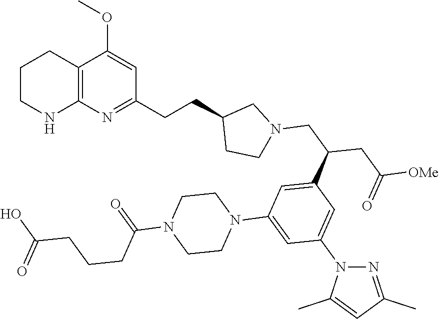 [SMILES: COC(=O)C[C@H](CN1CC[C@@H](CCc2cc(OC)c3c(n2)NCCC3)C1)c1cc(N2CCN(C(=O)CCCC(=O)O)CC2)cc(-n2nc(C)cc2C)c1] | 5-(4-(3-(3,5-dimethyl-1H-pyrazol-1-yl)-5-((S)-4-methoxy- 1-((R)-3-(2-(4-methoxy-5,6,7,8-tetrahydro-1,8- naphthyridin-2-yl)ethyl)pyrrolidin-1-yl)-4-oxobutan-2- yl)phenyl)piperazin-1-yl)-5-oxopentanoic acid; |
| 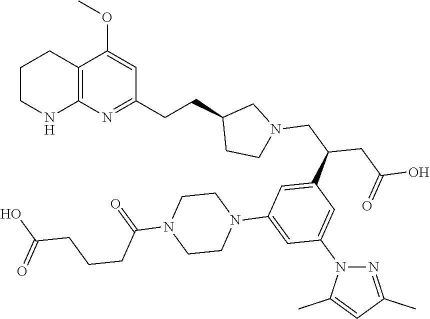 [SMILES: COc1cc(CC[C@@H]2CCN(C[C@@H](CC(=O)O)c3cc(N4CCN(C(=O)CCCC(=O)O)CC4)cc(-n4nc(C)cc4C)c3)C2)nc2c1CCCN2] | 5-(4-(3-((S)-1-carboxy-3-((R)-3-(2-(4-methoxy-5,6,7,8- tetrahydro-1,8-naphthyridin-2-yl)ethyl)pyrrolidin-1- yl)propan-2-yl)-5-(3,5-dimethyl-1H-pyrazol-1- yl)phenyl)piperazin-1-yl)-5-oxopentanoic acid; |
| 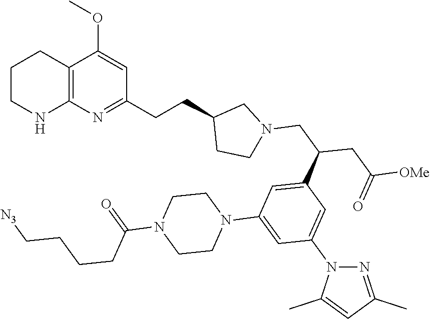 [SMILES: COC(=O)C[C@H](CN1CC[C@@H](CCc2cc(OC)c3c(n2)NCCC3)C1)c1cc(N2CCN(C(=O)CCCCN=[N+]=[N-])CC2)cc(-n2nc(C)cc2C)c1] | methyl (S)-3-(3-(4-(5-azidopentanoyl)piperazin-1-yl)-5- (3,5-dimethyl-1H-pyrazol-1-yl)phenyl)-4-((R)-3-(2-(4- methoxy-5,6,7,8-tetrahydro-1,8-naphthyridin-2- yl)ethyl)pyrrolidin-1-yl)butanoate; |
| 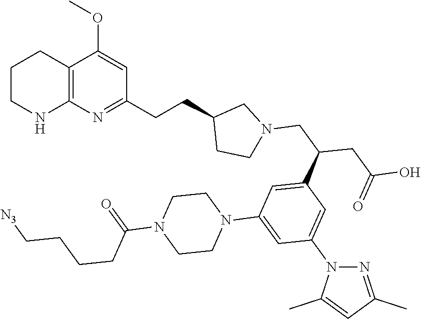 [SMILES: COc1cc(CC[C@@H]2CCN(C[C@@H](CC(=O)O)c3cc(N4CCN(C(=O)CCCCN=[N+]=[N-])CC4)cc(-n4nc(C)cc4C)c3)C2)nc2c1CCCN2] | (S)-3-(3-(4-(5-azidopentanoyl)piperazin-1-yl)-5-(3,5- dimethyl-1H-pyrazol-1-yl)phenyl)-4-((R)-3-(2-(4- methoxy-5,6,7,8-tetrahydro-1,8-naphthyridin-2- yl)ethyl)pyrrolidin-1-yl)butanoic acid; |
| 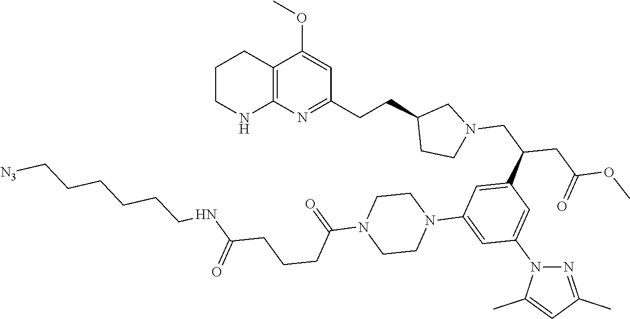 [SMILES: COC(=O)C[C@H](CN1CC[C@@H](CCc2cc(OC)c3c(n2)NCCC3)C1)c1cc(N2CCN(C(=O)CCCC(=O)NCCCCCCN=[N+]=[N-])CC2)cc(-n2nc(C)cc2C)c1] | methyl (S)-3-(3-(4-(5-((6-azidohexyl)amino)-5- oxopentanoyl)piperazin-1-yl)-5-(3,5-dimethyl-1H- pyrazol-1-yl)phenyl)-4-((R)-3-(2-(4-methoxy-5,6,7,8- tetrahydro-1,8-naphthyridin-2-yl)ethyl)pyrrolidin-1- yl)butanoate; |
| 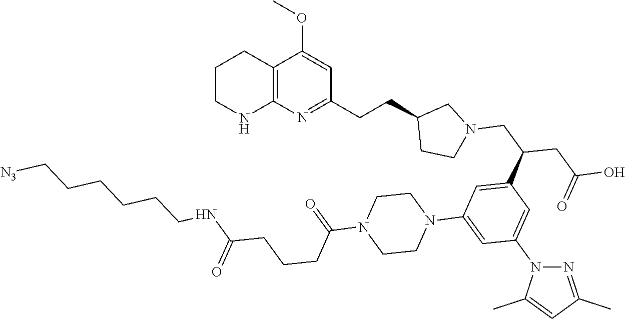 [SMILES: COc1cc(CC[C@@H]2CCN(C[C@@H](CC(=O)O)c3cc(N4CCN(C(=O)CCCC(=O)NCCCCCCN=[N+]=[N-])CC4)cc(-n4nc(C)cc4C)c3)C2)nc2c1CCCN2] | (S)-3-(3-(4-(5-((6-azidohexyl)amino)-5- oxopentanoyl)piperazin-1-yl)-5-(3,5-dimethyl-1H- pyrazol-1-yl)phenyl)-4-((R)-3-(2-(4-methoxy-5,6,7,8- tetrahydro-1,8-naphthyridin-2-yl)ethyl)pyrrolidin-1- yl)butanoic acid; |
| 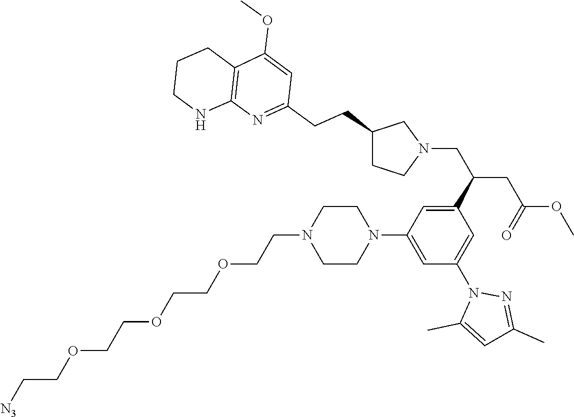 | methyl (S)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-((R)-3-(2-(4- methoxy-5,6,7,8-tetrahydro-1,8-naphthyridin-2- yl)ethyl)pyrrolidin-1-yl)butanoate |
| 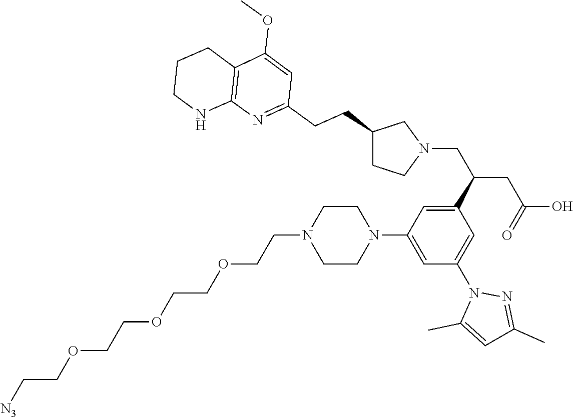 | (S)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-((R)-3-(2-(4- methoxy-5,6,7,8-tetrahydro-1,8-naphthyridin-2- yl)ethyl)pyrrolidin-1-yl)butanoic acid |
| 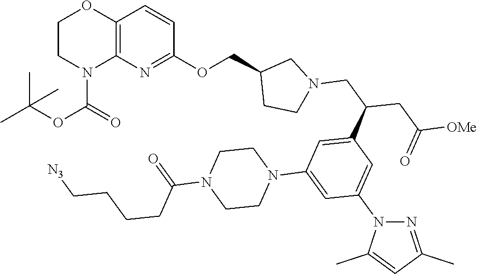 [SMILES: COC(=O)C[C@H](CN1CC[C@@H](COc2ccc3c(n2)N(C(=O)OC(C)(C)C)CCO3)C1)c1cc(N2CCN(C(=O)CCCCN=[N+]=[N-])CC2)cc(-n2nc(C)cc2C)c1] | tert-butyl 7-(((R)-1-((S)-2-(3-(4-(5- azidopentanoyl)piperazin-1-yl)-5-(3,5-dimethyl-1H- pyrazol-1-yl)phenyl)-4-methoxy-4-oxobutyl)pyrrolidin-3- yl)methoxy)-3,4-dihydro-1,8-naphthyridine-1(2H)- carboxylate |
| 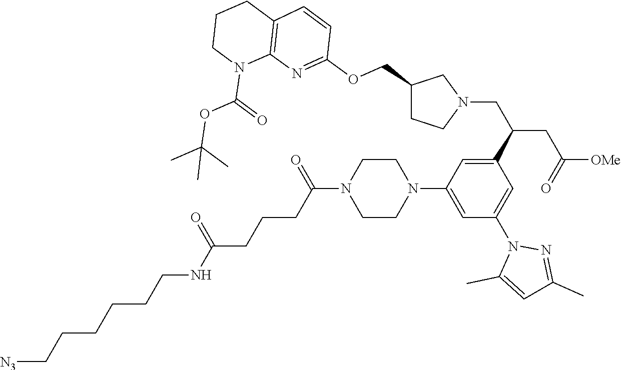 | tert-butyl 7-(((R)-1-((S)-2-(3-(4-(5-((6- azidohexyl)amino)-5-oxopentanoyl)piperazin-1-yl)-5- (3,5-dimethyl-1H-pyrazol-1-yl)phenyl)-4-methoxy-4- oxobutyl)pyrrolidin-3-yl)methoxy)-3,4-dihydro-1,8- naphthyridine-1(2H)-carboxylate |
| 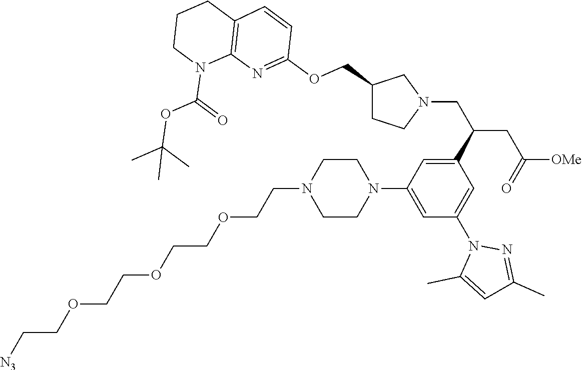 | tert-butyl 7-(((R)-1-((S)-2-(3-(4-(2-(2-(2-(2- azidoethoxy)ethoxy)ethoxy)ethyl)piperazin-1-yl)-5-(3,5- dimethyl-1H-pyrazol-1-yl)phenyl)-4-methoxy-4- oxobutyl)pyrrolidin-3-yl)methoxy)-3,4-dihydro-1,8- naphthyridine-1(2H)-carboxylate |
| 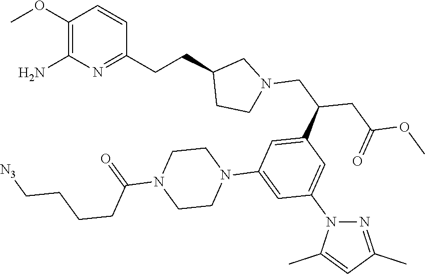 [SMILES: COC(=O)C[C@H](CN1CC[C@@H](CCc2ccc(OC)c(N)n2)C1)c1cc(N2CCN(C(=O)CCCCN=[N+]=[N-])CC2)cc(-n2nc(C)cc2C)c1] | methyl (S)-4-((R)-3-(2-(6-amino-5- - 2-yl)ethyl)pyrrolidin- 1-yl)-3-(3-(4-(5-azidopentanoyl)piperazin-1-yl)-5-(3,5- dimethyl-1H-pyrazol-1-yl)phenyl)butanoate |
| 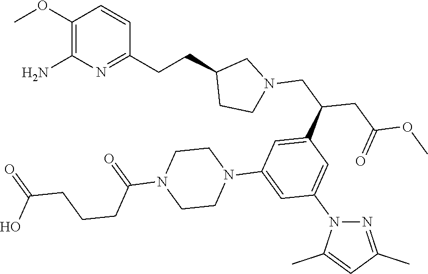 [SMILES: COC(=O)C[C@H](CN1CC[C@@H](CCc2ccc(OC)c(N)n2)C1)c1cc(N2CCN(C(=O)CCCC(=O)O)CC2)cc(-n2nc(C)cc2C)c1] | 5-(4-(3-((S)-1-((R)-3-(2-(6-amino-5-methoxypyridin-2- yl)ethyl)pyrrolidin-1-yl)-4-methoxy-4-oxobutan-2-yl)-5- (3,5-dimethyl-1H-pyrazol-1-yl)phenyl)piperazin-1-yl)-5- oxopentanoic acid |
| 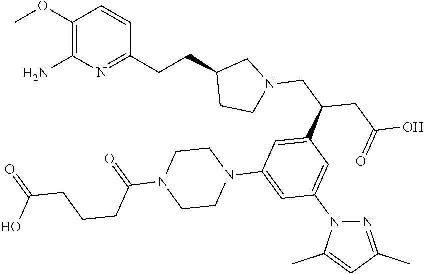 [SMILES: COc1ccc(CC[C@@H]2CCN(C[C@@H](CC(=O)O)c3cc(N4CCN(C(=O)CCCC(=O)O)CC4)cc(-n4nc(C)cc4C)c3)C2)nc1N] | 5-(4-(3-((S)-1-((R)-3-(2-(6-amino-5-methoxypyridin-2- yl)ethyl)pyrrolidin-1-yl)-3-carboxypropan-2-yl)-5-(3,5- dimethyl-1H-pyrazol-1-yl)phenyl)piperazin-1-yl)-5- oxopentanoic acid |
| 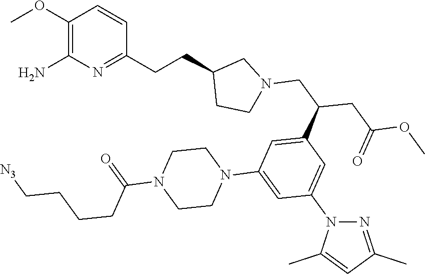 [SMILES: COC(=O)C[C@H](CN1CC[C@@H](CCc2ccc(OC)c(N)n2)C1)c1cc(N2CCN(C(=O)CCCCN=[N+]=[N-])CC2)cc(-n2nc(C)cc2C)c1] | methyl (S)-4-((R)-3-(2-(6-amino-5-methoxypyridin-2- yl)ethyl)pyrrolidin-1-yl)-3-(3-(4-(5- azidopentanoyl)piperazin-1-yl)-5-(3,5-dimethyl-1H- pyrazol-1-yl)phenyl)butanoate |
| 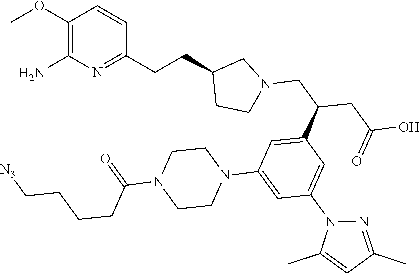 [SMILES: COc1ccc(CC[C@@H]2CCN(C[C@@H](CC(=O)O)c3cc(N4CCN(C(=O)CCCCN=[N+]=[N-])CC4)cc(-n4nc(C)cc4C)c3)C2)nc1N] | (S)-4-((R)-3-(2-(6-amino-5-methoxypyridin-2- yl)ethyl)pyrrolidin-1-yl)-3-(3-(4-(5- azidopentanoyl)piperazin-1-yl)-5-(3,5-dimethyl-1H- pyrazol-1-yl)phenyl)butanoic acid |
| 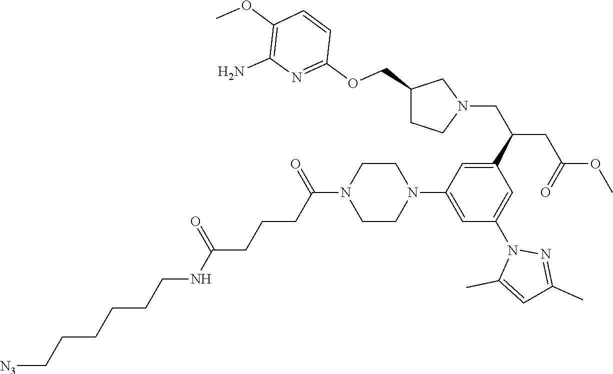 | methyl (S)-4-((R)-3-(2-(6-amino-5-methoxypyridin-2- yl)ethyl)pyrrolidin-1-yl)-3-(3-(4-(5-((6- azidohexyl)amino)-5-oxopentanoyl)piperazin-1-yl)-5- (3,5-dimethyl-1H-pyrazol-1-yl)phenyl)butanoate |
| 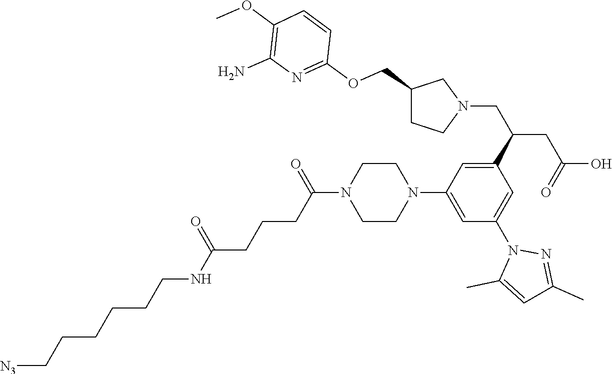 | (S)-4-((R)-3-(2-(6-amino-5-methoxypyridin-2- yl)ethyl)pyrrolidin-1-yl)-3-(3-(4-(5-((6- azidohexyl)amino)-5-oxopentanoyl)piperazin-1-yl)-5- (3,5-dimethyl-1H-pyrazol-1-yl)phenyl)butanoic acid |
| 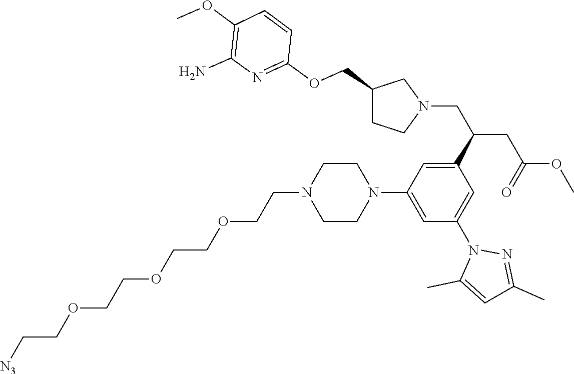 | methyl (S)-4-((R)-3-(2-(6-amino-5-methoxypyridin-2- yl)ethyl)pyrrolidin-1-yl)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)butanoate |
| 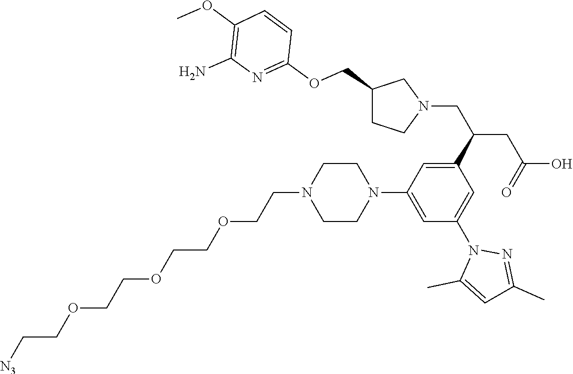 | (S)-4-((R)-3-(2-(6-amino-5-methoxypyridin-2- yl)ethyl)pyrrolidin-1-yl)-3-(3-(4-(2-(2-(2-(2- azidoethoxy)ethoxy)ethoxy)ethyl)piperazin-1-yl)-5-(3,5- dimethyl-1H-pyrazol-1-yl)phenyl)butanoic acid |
| | and salts thereof. |

In another embodiment, the compound of Formula (IV) is selected from the group consisting of,

| | |
|---|---|
| 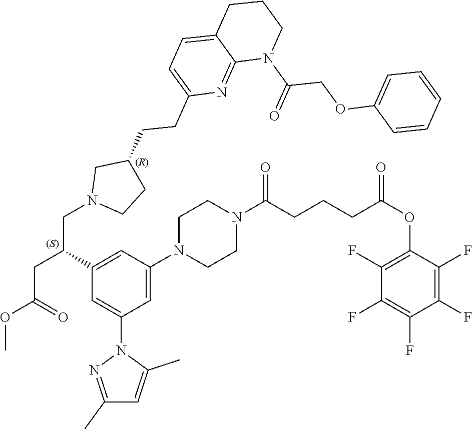 | perfluorophenyl 5-(4-(3-(3,5-dimethyl-1H-pyrazol- 1-yl)-5-((S)-4-methoxy-4-oxo-1-((R)-3-(2-(8-(2- phenoxyacetyl)-5,6,7,8-tetrahydro-1,8- naphthyridin-2-yl)ethyl)pyrrolidin-1-yl)butan-2- yl)phenyl)piperazin-1-yl)-5-oxopentanoate |
| 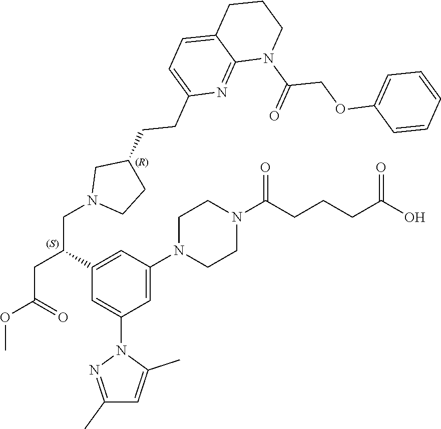 | 5-(4-(3-(3,5-dimethyl-1H-pyrazol-1-yl)-5-((S)-4- methoxy-4-oxo-1-((R)-3-(2-(8-(2-phenoxyacetyl)- 5,6,7,8-tetrahydro-1,8-naphthyridin-2- yl)ethyl)pyrrolidin-1-yl)butan-2- yl)phenyl)piperazin-1-yl)-5-oxopentanoic acid |
| and salts thereof. | |

B. Alpha-v-Beta-6 (αvβ6) Integrin Ligands

In another aspect, the present application provides modified integrin-modifying compounds in a conjugated to a carrier group suitable for conjugation to or incorporation into an oligonucleotide. In some embodiments, the present disclosure provides a compound of the Formula (X),

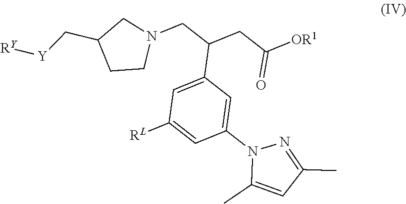

or a salt thereof, wherein:
R¹, R^{Y} and Y are as defined for Formula (IV); and
R^{L} is —N(R³)(R⁴), —O(R⁵), —S(R⁵), or —R⁵, wherein
   R³ and R⁴ are either
      (i) R³ is hydrogen or C₁₋₆alkyl and R⁴ is R⁵; or
      (ii) R³ and R⁴ taken together with the nitrogen atom to which they are attached form a 4-8 membered monocyclic heterocyclyl group that is substituted with R⁵;
   and
   R⁵ is -L-ZZ-L′-R^{T} wherein
      L and L′ are independently -L¹-[G-L²]_{q}-G-L³-*, wherein
         * is the bond to ZZ;
         q is 0 or an integer selected from 1-25; (e.g., 1-20, or 1-15);
         L¹ is a bond or -B-A-;
         each L² is independently -A-B-A-;
         L³ is a bond or -A-B-A-;
         each G is independently -D-E-F-, wherein D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
         each A is independently a bond, —O—, —S—, or —N(R^{N})—.
         each B is independently a bond, CH₂, C(O), C(S), C(NR^{N}), (O), S(O)₂, P(O)(OH), P(S)(OH), or P(S)(SH);
         each R^{N} is independently hydrogen or C₁₋₆alkyl, or two R^{N} within an -A-B-A- group taken together with the atoms to which they are connected from a 4-8 membered heterocyclyl; and
   ZZ is -A′-B′-A′- or a linking group formed by a reactive pair, wherein
      each A′ is independently a bond, —O—, —S—, or —N(R^{N3})—;
      each B′ is independently a bond, CH₂, C(O), C(S), C(NR^{N3}), —C═N—, S(O), S(O)₂, P(O)(OH), P(S)(OH), or P(S)(SH); and
      each R^{N3} is independently hydrogen or C₁₋₆alkyl, or two R^{N3} within the -A′-B′-A′- group taken together with the atoms to which they are connected from a 4-8 membered heterocyclyl;
   R^{T} is R^{T1} or -G⁰-OR^{T1}, wherein
      G⁰ is absent or -D⁰-E⁰-F⁰-, wherein D⁰, E⁰, and F⁰ are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
      and
      R^{T1} is hydrogen, a hydroxyl protecting group, a phosphorous coupling group, or -L^{K}-S^{S}, or -L^{L}-oligonucleotide, wherein
         L^{K} is a support linking group;
         L^{L} is an oligonucleotide linking group; and
         S^{S} is a solid support, —OR^{SS} or —N(R^{SS})₂, or hydrogen, wherein each R^{SS} is independently hydrogen or C₁₋₆alkyl.
and
each R group is independently selected from the group consisting of R′, C₁₋₆alkyl, C₁₋₆ haloalkyl, C₂₋₆alkenyl, C₂₋₆alkynyl, C₃₋₈cycloalkyl, heterocyclyl, aryl, heteroaryl, C₃₋₈cycloalkylC₁₋₆alkyl, heterocyclylC₁₋₆alkyl, aryl C₁₋₆alkyl, heteroarylC₁₋₆alkyl, each of which, other than R′, is optionally substituted with 1, 2, or 3 R′ groups, wherein
   each R′ is independently halogen, cyano, azido, nitro, —N(R^{b})₂, —O(R^{a}), —S(R⁰), —C(O)OR⁰,
   C(O)R⁰, —C(O)N(R⁰)₂, —C(NR⁰)OR, —C(NR⁰)R⁰, —C(NR⁰)N(R⁰)₂, —C(S)OR⁰, —C(S)R⁰, —C(S)N(R⁰)₂, —S(O)₂R⁰, —S(O)₂OR⁰, —S(O)₂N(R⁰)₂, —N(R⁰)C(O)OR⁰, —N(R⁰)C(O)R⁰, —N (R⁰)C(O)N(R⁰)₂, —N(R⁰)S(O)₂R⁰, —N(R⁰)S(O)₂OR⁰, —N(R⁰)S(O)₂N(R⁰)₂, —OC(O)OR⁰, —OC(O)R⁰, —OC(O)N(R⁰)₂, —OS(O)₂R⁰, —OS(O)₂OR⁰, —OS(O)₂N(R⁰)₂, or —SC(O)R⁰, wherein
      each R⁰ is independently hydrogen or C₁₋₆alkyl; each R^{a} is independently hydrogen, C₁₋₆alkyl, or a hydroxyl protecting group; and each R^{b} is independently hydrogen, C₁₋₆alkyl, or a nitrogen protecting group
provided that in each -D-E-F- group, at least one of D, E, and F is not a bond; and R^{L} is not N-morpholinyl.

Embodiment for the variables of Formula (X) that are the same as Formula (IV) are as described above for Formula (IV).

In some embodiments of Formula (X), in —N(R³)(R⁴), R³ and R⁴ do not form a morpholino ring.

In some embodiments of Formula (X), in each -A-B-A- group, B is only a bond when one of the A groups is not a bond.

### R^{T} Embodiments, Formula (X)

In some embodiments of Formula (X), R^{T} is R^{T1} (e.g., -L^{L}-oligonucleotide).

In some embodiments of Formula (X), R^{T} is -G⁰-OR^{T1}, wherein R^{T1} is as defined for Formula (X) and G⁰ is selected from:
(a) G⁰ is absent or -D⁰-E⁰-F⁰-, wherein D⁰, E⁰, and F⁰ are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
(b) G⁰ is -D⁰-E⁰-F⁰-, wherein
   D⁰ and F⁰ are independently a bond or C₁₋₁₀alkyl optionally substituted with 1, 2, 3, or 4 R groups; and
   E⁰ is C₃₋₁₀cycloalkyl, 3-10 membered heterocyclyl, aryl, or heteroaryl, each of which is optionally substituted with 1, 2, 3, or 4 R groups;
(c) G⁰ is -D⁰-E⁰-F⁰-, wherein
   D⁰ and F⁰ are independently a bond or C₁₋₁₀alkyl optionally substituted with 1, 2, 3, or 4 R groups; and
   E⁰ is 3-10 membered heterocyclyl optionally substituted with 1, 2, 3, or 4 R groups; and
(d) G⁰ is 3-10 membered heterocyclyl optionally substituted with 1 or 2 R groups; examples include

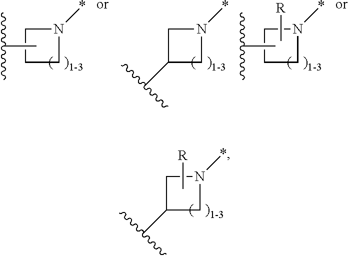

G⁰ is tetrahydrofuranyl, pyrrolidinyl, piperidinyl, piperazinyl, or morpholinyl, each optionally substituted with 1 or 2 R groups; examples include:

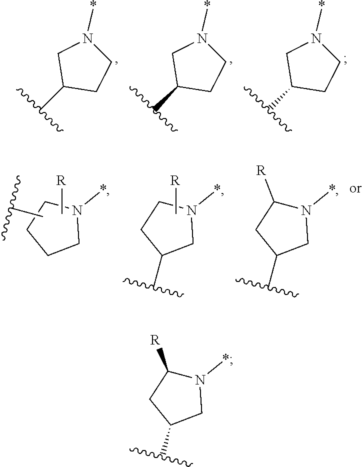

and
(e) G⁰ is 3-10 membered-heterocyclyl-C₁₋₁₀alkyl, optionally substituted with 1, 2, 3, or 4 R groups; examples include,

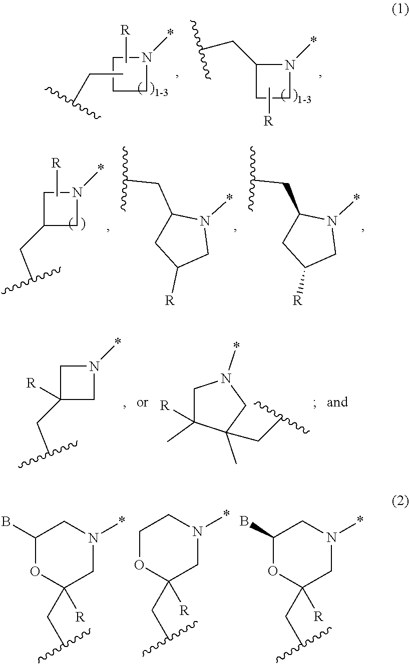

(f) G⁰ is C₁₋₁₀alkyl, C₂₋₁₀alkenyl, C₂₋₁₀alkynyl, each of which is optionally substituted with 1, 2, 3, or 4 R groups (e.g., 1 or 2 R groups);
(g) G⁰ is C₁₋₁₀alkyl or C₂₋₁₀alkenyl, each of which is optionally substituted with 1 or 2 R groups;
(h) G⁰ is C₁₋₁₀alkyl optionally substituted with 1 or 2 R groups;
(i) G⁰ is C₁₋₁₀alkyl, optionally substituted with —O(R^{a}), wherein R^{a} is independently hydrogen, C₁₋₆alkyl, or a hydroxyl protecting group; e.g.,

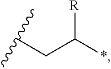

(j) G⁰ is C₁₋₁₀alkyl,
and
(k) G⁰ is absent (a bond);
   wherein * represents the bond to L′, the broken bond represents the bond to OR^{T1}, and R is —C₁₋₆alkyl-OR^{a} or —OR^{a}, wherein R^{a} is independently hydrogen, C₁₋₆alkyl, or a hydroxyl protecting group;

### -L′- Embodiments, Formula (X)

In some embodiments of Formula (X), -L′- is *-G-L′-, wherein * is the bond to ZZ; and
(a) L¹ is a bond or -B-A-, wherein
   A is a bond, —O—, —S—, or —N(R^{N})—;
   B is a bond, CH₂, C(O), C(S), C(NR^{N}), S(O), S(O)₂, P(O)(OH), P(S)(OH), or P(S)(SH);
   each R^{N} is independently hydrogen or C₁₋₆alkyl; and
   G is -D-E-F-, wherein D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
(b) L¹ is a bond or -B-A-, wherein
   A is a bond, —O—, —S—, or —N(R^{N})—;
   B is a bond, CH₂, C(O), C(S), C(NR^{N}), S(O), S(O)₂, P(O)(OH), P(S)(OH), or P(S)(SH);
   each R^{N} is independently hydrogen or C₁₋₆alkyl; and
   G is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
(c) L¹ is a bond, C(O), C(S), S(O)₂, P(O)(OH), or P(S)(OH); and
   G is C₁₋₁₀ alkyl, C₂₋₁₀alkenyl, C₂₋₁₀alkynyl, each of which is optionally substituted with 1, 2, 3, or 4 R groups;
(d) L¹ is a bond, C(O), C(S), S(O)₂, P(O)(OH), or P(S)(OH); and
   G is C₁₋₁₀ alkyl, C₂₋₁₀alkenyl, C₂₋₁₀alkynyl, each of which is optionally substituted with 1, 2, 3, or 4 R groups;
or
(e) L¹ is a bond, C(O), C(S), S(O)₂, P(O)(OH), or P(S)(OH); and G is C₁₋₁₀alkyl.

In some embodiments of Formula (X), -L′- is *-G-[L²-G]_{q}-L¹-, wherein * is the bond to ZZ; q, is 1, 2, 3, 4, 5, 6, 7, 8, 9, or 10 (e.g., an integer from 1 to 8, an integer from 1 to 5, or an integer from 1 to 3); and
(a) L¹ is a bond or -B-A-;
   each L² is independently -A-B-A-;
   each A is independently a bond, —O—, —S—, or —N(R^{N})—;
   each B is independently a bond, CH₂, C(O), C(S), C(NR^{N}), S(O), S(O)₂, P(O)(OH), P(S)(OH), or P(S)(SH);
   each R^{N} is independently hydrogen or C₁₋₆alkyl;
   each G is independently -D-E-F-, wherein D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
(b) L¹ is a bond, CH₂, C(O), C(S), C(NR^{N}), S(O), S(O)₂, P(O)(OH), P(S)(OH), or P(S)(SH);
   each L² is independently -A-B-A-;
      each A is independently a bond, —O—, —S—, or —N(R^{N})—;
      each B is independently a bond, CH₂, C(O), C(S), C(NR^{N}), S(O), S(O)₂, P(O)(OH), P(S)(OH), or P(S)(SH);
      each R^{N} is independently hydrogen or C₁₋₆alkyl, and
      each G is independently a bond, C₁₋₁₀alkyl, optionally substituted with 1, 2, 3, or 4 R groups.
(c) L¹ is a bond, CH₂, C(O), C(S), C(NR^{N}), S(O), S(O)₂, P(O)(OH), P(S)(OH), or P(S)(SH);
   each L² is independently -A-B-A-;
   each A is independently a bond, —O—, —S—, or —N(R^{N})—, wherein R^{N} is hydrogen or C₁₋₆ alkyl
   each B is independently a bond, CH₂, C(O), C(S), S(O)₂, P(O)(OH), or P(S)(OH);
   each G is independently C₁₋₁₀alkyl, optionally substituted with 1, 2, 3, or 4 R groups
(d) L¹ is a bond, CH₂, C(O), C(S), S(O)₂, P(O)(OH), or P(S)(OH)
   each L² is independently -A-B-A-;
   each A is independently a bond, —O—, —S—, or —N(R^{N})—, wherein R^{N} is hydrogen or C₁₋₆ alkyl
   each B is independently a bond, CH₂, C(O), C(S), S(O)₂, P(O)(OH), or P(S)(OH);
   each G is independently C₁₋₁₀alkyl, optionally substituted with 1, 2, 3, or 4 R groups;
or
(e) L¹ is a bond, CH₂, C(O), S(O)₂, P(O)(OH), or P(S)(OH);
   each L² is independently -A-B-A-;
      each A is independently a bond, —O—, —S—, or —N(R^{N})—, wherein R^{N} is hydrogen or C₁₋₆ alkyl
      each B is independently a bond, CH₂, C(O), C(S), S(O)₂, P(O)(OH), or P(S)(OH);
      each G is independently C₁₋₁₀alkyl;

In some embodiments of Formula (X), -L′- is -[G-L²]_{q}-G-L³-*, wherein * is the bond to ZZ; and
(a) q is 0, 1, 2, 3, 4, or 5;
   each L² is independently -A-B-A-;
   L³ is a bond or -A-B-A-;
   each A is independently a bond, —O—, —S—, or —N(R^{N})—;
   each B is independently a bond, CH₂, C(O), C(S), C(NR^{N}), S(O), S(O)₂, P(O)(OH), P(S)(OH), or P(S)(SH);
   each R^{N} is independently hydrogen or C₁₋₆alkyl;
   each G is independently C₁₋₁₀alkyl, C₂₋₁₀alkenyl, C₂₋₁₀alkynyl, each of which is optionally substituted with 1, 2, 3, or 4 R groups;
(b) q is 0, 1, 2, or 3;
   each L² is independently a bond, C(O)O, OC(O), C(O)(NR^{N}), N(R^{N})C(O), SO₂N(R^{N}), N(R^{N})SO₂, OP(O)(OH), OP(S)(OH), P(O)(OH)O, P(S)(OH)O, OP(O)(OH)O, or OP(S)(OH)O, wherein each R^{N} is independently hydrogen or C₁₋₆ alkyl;
   each G is independently C₁₋₁₀alkyl, C₂₋₁₀alkenyl, C₂₋₁₀alkynyl, each of which is optionally substituted with 1 or 2 R groups;
(c) q is 0, 1, 2, or 3;
   each L² is independently a bond, C(O)O, OC(O), C(O)(NR^{N}), N(R^{N})C(O), OP(O)(OH)O, or OP(S)(OH)O, wherein each R^{N} is independently hydrogen or C₁₋₆ alkyl;
   each G is independently C₁₋₁₀alkyl or C₂₋₁₀alkenyl, each of which is optionally substituted with 1 or 2 R groups;
(d) q is 0, 1, 2, or 3 (e.g., q is 0, 1, or 2; or 0 or 1; or 0; or 1; or 2);
   each L² is independently C(O)O or OC(O);
   each G is independently C₁₋₁₀alkyl, each of which is optionally substituted with 1 or 2 R groups;
(e) q is 0, 1, 2, or 3 (e.g., q is 0, 1, or 2; or 0 or 1; or 0; or 1; or 2);
   each L² is independently C(O)(NR^{N}) or N(R^{N})C(O), wherein each R^{N} is independently hydrogen or C₁₋₆alkyl;
   each G is independently C₁₋₁₀alkyl, each of which is optionally substituted with 1 or 2 R groups;
(f) q is 0, 1, 2, or 3 (e.g., q is 0, 1, or 2; or 0 or 1; or 0; or 1; or 2);
   each L² is independently OP(O)(OH)O, or OP(S)(OH)O (e.g., each is OP(O)(OH)O);
   each G is independently C₁₋₁₀alkyl, each of which is optionally substituted with 1 or 2 R groups;
or
(g) q is 0, 1, 2, or 3 (e.g., q is 0, 1, or 2; or 0 or 1; or 0; or 1; or 2);
   each L² is a bond;
      each G is independently C₁₋₁₀alkyl, each of which is optionally substituted with 1 or 2 R groups.

In some embodiments, -L′- is *-L³-G-L¹-, wherein * is the bond to ZZ; and
(a) L¹ and L³ are independently -A-B-A-;
   each G is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, or 3 R groups;
      each A is independently a bond, —O—, —S—, or —N(R^{N})—, wherein R^{N} is hydrogen or C₁₋₆ alkyl; and
      each B is independently a bond, C(O), C(S), S(O)₂, P(O)(OH), or P(S)(OH);
(b) L³ is —C(O)O— or C(O)N(R^{N})—, wherein R^{N} is hydrogen or C₁₋₆alkyl;
   L¹ is —OP(O)(OH)O— or —OP(S)(OH)O—; and
   each G is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, or 3 R groups;
(c) L³ is —C(O)O— or C(O)N(R^{N})—, wherein R^{N} is hydrogen or C₁₋₆alkyl;
   L¹ is —OP(O)(OH)O— or —OP(S)(OH)O—; and
   each G is independently C₃₋₁₀cycloalkyl, 3-10 membered heterocyclyl, aryl, or heteroaryl, each of which is optionally substituted with one R group;
(d) L³ is —C(O)O— or C(O)N(R^{N})—, wherein R^{N} is hydrogen or C₁₋₆alkyl;
   L¹ is —OP(O)(OH)O— or —OP(S)(OH)O—; and
   each G is independently C₃₋₁₀cycloalkyl or 3-10 membered heterocyclyl;
(e) L³ is —C(O)O— or C(O)N(R^{N})—, wherein R^{N} is hydrogen or C₁₋₆alkyl;
   L¹ is —OP(O)(OH)O— or —OP(S)(OH)O—; and
   each G¹ is independently C₃₋₁₀cycloalkyl or 3-10 membered heterocyclyl; and

In some embodiments, -L′- is

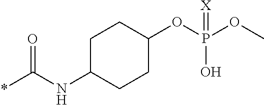

wherein X is O or S (e.g., S).

In some embodiments, -L′- is *-G-, wherein * is the bond to ZZ; and G is C₁₋₁₀alkyl is optionally substituted with 1 or 2 R groups.

In embodiments of Formula (X), including embodiments of Formulae (x-a) through (x-x), -L′- is -L¹-[G-L²]_{q}-G-L³-*, wherein * is the bond to ZZ;

In some embodiments, -L′- is -L′-G-L³-*, wherein * is the bond to ZZ;

In some embodiments, -L′- is -L¹-[G-L²]_{q}-G-L³-*, wherein * is the bond to ZZ;
q is 0, 1, 2, 3, 4, or 5;
L¹ is a bond or -B-A-;
each L² is independently -A-B-A-;
L³ is a bond or -A-B-A-;
each A is independently a bond, —O—, —S—, or —N(R^{N})—;
each B is independently a bond, CH₂, C(O), C(S), C(NR^{N}), S(O), S(O)₂, P(O)(OH), P(S)(OH), or P(S)(SH);
each R^{N} is independently hydrogen or C₁₋₆alkyl; and
each G is independently C₁₋₁₀alkyl, C₂₋₁₀alkenyl, C₂₋₁₀alkynyl, each of which is optionally substituted with 1, 2, 3, or 4 R groups.

In some embodiments, -L′- is -L¹-[G-L²]_{q}-G-*, wherein * is the bond to ZZ;
q is 0, 1, 2, or 3;
L¹ is a bond or -B-A-;
each L² is independently a bond, C(O)O, OC(O), C(O)(NR^{N}), N(R^{N})C(O), SO₂N(R^{N}), N(R^{N})SO₂, OP(O)(OH), OP(S)(OH), P(O)(OH)O, P(S)(OH)O, OP(O)(OH)O, or OP(S)(OH)O, wherein each R^{N} is independently hydrogen or C₁₋₆alkyl; and
each G is independently C₁₋₁₀alkyl, C₂₋₁₀alkenyl, C₂₋₁₀alkynyl, each of which is optionally substituted with 1 or 2 R groups.

In some embodiments, -L′- is -L¹-[G-L²]_{q}-G-*, wherein * is the bond to ZZ;
q is 0, 1, 2, or 3;
L¹ is a bond or -B-A-;
each L² is independently a bond, C(O)O, OC(O), C(O)(NR^{N}), N(R^{N})C(O), OP(O)(OH)O, or OP(S)(OH)O, wherein each R^{N} is independently hydrogen or C₁₋₆ alkyl;
each G is independently C₁₋₁₀alkyl or C₂₋₁₀alkenyl, each of which is optionally substituted with 1 or 2 R groups.

In some embodiments, -L′- is -[G-L²]_{q}-G-*, wherein * is the bond to ZZ; and
(a) q is 0, 1, 2, or 3 (e.g., q is 0, 1, or 2; or 0 or 1; or 0; or 1; or 2);
   each L² is independently C(O)O or OC(O);
   each G is independently C₁₋₁₀alkyl, each of which is optionally substituted with 1 or 2 R groups;
(b) q is 0, 1, 2, or 3 (e.g., q is 0, 1, or 2; or 0 or 1; or 0; or 1; or 2);
   each L² is independently C(O)(NR^{N}) or N(R^{N})C(O), wherein each R^{N} is independently hydrogen or C₁₋₆alkyl; and
   each G is independently C₁₋₁₀alkyl, each of which is optionally substituted with 1 or 2 R groups;
(c) q is 0, 1, 2, or 3 (e.g., q is 0, 1, or 2; or 0 or 1; or 0; or 1; or 2);
   each L² is independently OP(O)(OH)O, or OP(S)(OH)O (e.g., each is OP(O)(OH)O); and each G is independently C₁₋₁₀alkyl, each of which is optionally substituted with 1 or 2 R groups;
or
(d) q is 0, 1, 2, or 3 (e.g., q is 0, 1, or 2; or 0 or 1; or 0; or 1; or 2);
   each L² is a bond; and each G is independently C₁₋₁₀alkyl, each of which is optionally substituted with 1 or 2 R groups.

In some embodiments, -L′- is —C₂₋₃₀alkyl-*, wherein * is the bond to ZZ, such as —C₅₋₂₀alkyl-* or —C₁₀₋₂₀alkyl-*.

In some embodiments, -L′- is —C(O)—C₂₋₃₀alkyl-*, wherein * is the bond to ZZ, such as —C(O)—C₅₋₂₀alkyl-* or —C(O)—C₁₀₋₂₀alkyl-*.

### L′-R^{T} Embodiments

In some embodiments, -L′-R^{T} is

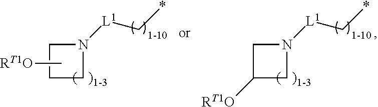

wherein
* is the bond to ZZ;
L¹ is a bond, C(O), C(S), S(O)₂, P(O)(OH), or P(S)(OH); and
   R^{T1} is as defined for Formula (X).

In some embodiments, -L′-R^{T} is

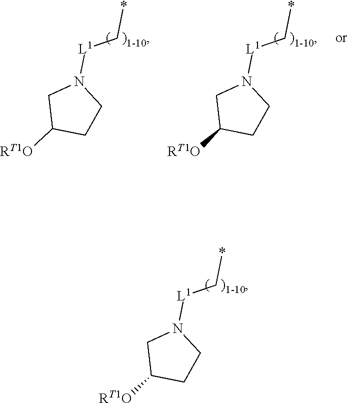

wherein
* is the bond to ZZ;
L¹ is a bond, C(O), C(S), S(O)₂, P(O)(OH), or P(S)(OH); and R^{T1} is as defined for Formula (X).

In some embodiments, -L′-R^{T} is

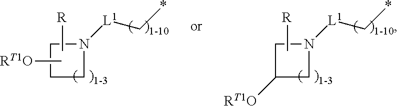

wherein * is the bond to ZZ;
L¹ is a bond, C(O), C(S), S(O)₂, P(O)(OH), or P(S)(OH);
R is —O(R^{a}) or —C₁₋₆alkyl-O(R^{a}), wherein R^{a} is hydrogen or a hydroxyl protecting group; and
R^{T1} is as defined for Formula (X).

In some embodiments, -L′-R^{T} is

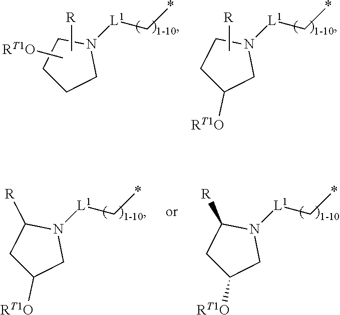

wherein * is the bond to ZZ;
L¹ is a bond, C(O), C(S), S(O)₂, P(O)(OH), or P(S)(OH);
R is —O(R^{a}) or —C₁₋₆alkyl-O(R^{a}), wherein R^{a} is hydrogen or a hydroxyl protecting group; and
R^{T1} is as defined for Formula (X).

In some embodiments, -L′-R^{T} is

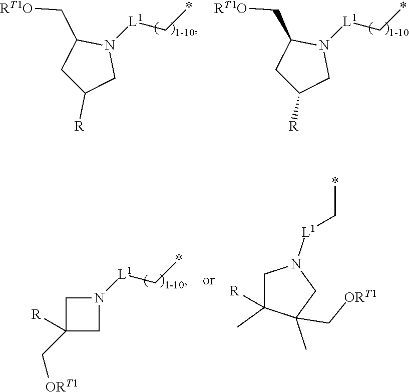

wherein* is the bond to ZZ;
   L¹ is a bond, C(O), C(S), S(O)₂, P(O)(OH), or P(S)(OH);
   R is —O(R^{a}) or —C₁₋₆alkyl-O(R^{a}), wherein R^{a} is hydrogen or a hydroxyl protecting group; and
   R^{T1} is as defined for Formula (X).

In some embodiments, -L′-R^{T} is

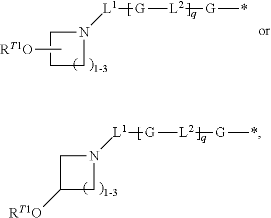

wherein
* is the bond to ZZ;
q is 1, 2, 3, 4, 5, 6, 7, 8, 9, or 10 (e.g., an integer from 1 to 8, an integer from 1 to 5, or an integer from 1 to 3);
each L² is
   (i) independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl; or
   (ii) independently selected from the group consisting of —C(O)O—, —OC(O)—, —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl; or
   (iii) is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl; or
   (iv) independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl; or
   (v) independently selected from the group consisting of —C(O)N(R^{N})—, —N(R^{N})C(O)—, —O—, and —N(R^{N})—, wherein each R^{N} is independently hydrogen or C₁₋₆alkyl;
   (vi) independently selected from the group consisting of —C(O)N(R^{N})— and —N(R^{N})C(O), wherein each R^{N} is independently hydrogen or C₁₋₆alkyl;
L¹ is a bond, C(O), C(S), S(O)₂, P(O)(OH), or P(S)(OH); and R^{T1} is as defined for Formula (X).

In some embodiments, -L′-R^{T} is

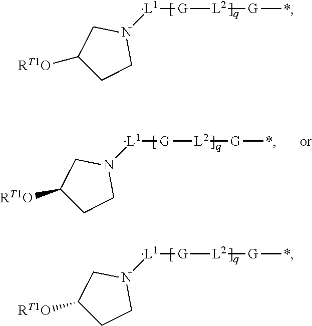

wherein
* is the bond to ZZ;
q is 1, 2, 3, 4, 5, 6, 7, 8, 9, or 10 (e.g., an integer from 1 to 8, an integer from 1 to 5, or an integer from 1 to 3);
each L² is
   (i) independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl; or
   (ii) independently selected from the group consisting of —C(O)O—, —OC(O)—, —C(O)N(R^{N})—, —N(R^{N})C(O)—, —OC(O)N(R^{N})—, —N(R^{N})COO, N(R^{N})C(O)N(R^{N})—, —O—, and —N(R^{N})—, wherein each R^{N} is independently hydrogen or C₁₋₆alkyl; or
   (iii) is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl; or
   (iv) independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl; or
   (v) independently selected from the group consisting of —C(O)N(R^{N})—, —N(R^{N})C(O)—, —O—, and —N(R^{N})—, wherein each R^{N} is independently hydrogen or C₁₋₆alkyl;
   (vi) independently selected from the group consisting of —C(O)N(R^{N})— and —N(R^{N})C(O), wherein each R^{N} is independently hydrogen or C₁₋₆alkyl;
L¹ is a bond, C(O), C(S), S(O)₂, P(O)(OH), or P(S)(OH); and
   R^{T1} is as defined for Formula (X). In some embodiments, -L′-R^{T} is

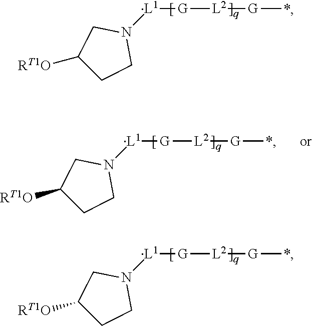

wherein
* is the bond to ZZ;
q is 1, 2, 3, 4, 5, 6, 7, 8, 9, or 10 (e.g., an integer from 1 to 8, an integer from 1 to 5, or an integer from 1 to 3);
each L² is
   (i) independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl; or
   (ii) independently selected from the group consisting of —C(O)O—, —OC(O)—, —C(O)N(R^{N})—, —N(R^{N})C(O)—, —OC(O)N(R^{N})—, —N(R^{N})COO, N(R^{N})C(O)N(R^{N})—, —O—, and —N(R^{N})—, wherein each R^{N} is independently hydrogen or C₁₋₆alkyl; or
   (iii) is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl; or
   (iv) independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl; or
   (v) independently selected from the group consisting of —C(O)N(R^{N})—, —N(R^{N})C(O)—, —O—, and —N(R^{N})—, wherein each R^{N} is independently hydrogen or C₁₋₆alkyl;
   (vi) independently selected from the group consisting of —C(O)N(R^{N})— and —N(R^{N})C(O), wherein each R^{N} is independently hydrogen or C₁₋₆alkyl;
L¹ is a bond, C(O), C(S), S(O)₂, P(O)(OH), or P(S)(OH); and
R^{T1} is hydrogen.

In some embodiments, -L′-R^{T} is

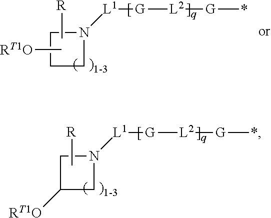

wherein * is the bond to ZZ;
q is 1, 2, 3, 4, 5, 6, 7, 8, 9, or 10 (e.g., 1-8; or 1-5; or 1-3);
each L² is
   (i) independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl; or
   (ii) independently selected from the group consisting of —C(O)O—, —OC(O)—, —C(O)N(R^{N})—, —N(R^{N})C(O)—, —OC(O)N(R^{N})—, —N(R^{N})COO, N(R^{N})C(O)N(R^{N})—, —O—, and —N(R^{N})—, wherein each R^{N} is independently hydrogen or C₁₋₆alkyl; or
   (iii) is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl; or
   (iv) independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl; or
   (v) independently selected from the group consisting of —C(O)N(R^{N})—, —N(R^{N})C(O)—, —O—, and —N(R^{N})—, wherein each R^{N} is independently hydrogen or C₁₋₆alkyl;
   (vi) independently selected from the group consisting of —C(O)N(R^{N})— and —N(R^{N})C(O), wherein each R^{N} is independently hydrogen or C₁₋₆alkyl;
L¹ is a bond, C(O), C(S), S(O)₂, P(O)(OH), or P(S)(OH);
R is —O(R^{a}) or —C₁₋₆alkyl-O(R^{a}), wherein R^{a} is hydrogen or a hydroxyl protecting group; and
   R^{T1} is as defined for Formula (X).

In some embodiments, -L′-R^{T} is

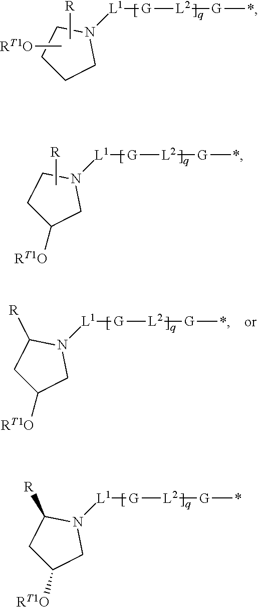

wherein * is the bond to ZZ;
q is 1, 2, 3, 4, 5, 6, 7, 8, 9, or 10 (e.g., an integer from 1 to 8, an integer from 1 to 5, or an integer from 1 to 3);
each L² is
   (i) independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl; or
   (ii) independently selected from the group consisting of —C(O)O—, —OC(O)—, —C(O)N(R^{N})—, —N(R^{N})C(O)—, —OC(O)N(R^{N})—, —N(R^{N})C₀₀, N(R^{N})C(O)N(R^{N})—, —O—, and —N(R^{N})—, wherein each R^{N} is independently hydrogen or C₁₋₆alkyl; or
   (iii) is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl; or
   (iv) independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl; or
   (v) independently selected from the group consisting of —C(O)N(R^{N})—, —N(R^{N})C(O)—, —O—, and —N(R^{N})—, wherein each R^{N} is independently hydrogen or C₁₋₆alkyl;
   (vi) independently selected from the group consisting of —C(O)N(R^{N})— and —N(R^{N})C(O), wherein each R^{N} is independently hydrogen or C₁₋₆alkyl;
L¹ is a bond, C(O), C(S), S(O)₂, P(O)(OH), or P(S)(OH);
R is —O(R^{a}) or —C₁₋₆alkyl-O(R^{a}), wherein R^{a} is hydrogen or a hydroxyl protecting group; and
R^{T1} is as defined for Formula (X).

In some embodiments, -L′-R^{T} is

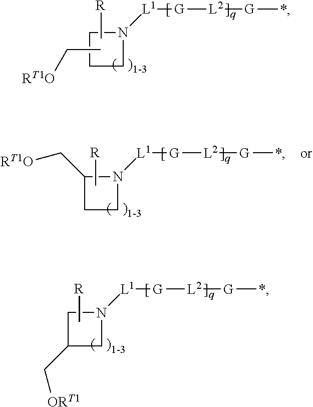

wherein* is the bond to ZZ;
q is 1, 2, 3, 4, 5, 6, 7, 8, 9, or 10 (e.g., an integer from 1 to 8, an integer from 1 to 5, or an integer from 1 to 3);
each L² is
   (i) independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl; or
   (ii) independently selected from the group consisting of —C(O)O—, —OC(O)—, —C(O)N(R^{N})—, —N(R^{N})C(O)—, —OC(O)N(R^{N})—, —N(R^{N})C₀₀, N(R^{N})C(O)N(R^{N})—, —O—, and —N(R^{N})—, wherein each R^{N} is independently hydrogen or C₁₋₆alkyl; or
   (iii) is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl; or
   (iv) independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl; or
   (v) independently selected from the group consisting of —C(O)N(R^{N})—, —N(R^{N})C(O)—, —O—, and —N(R^{N})—, wherein each R^{N} is independently hydrogen or C₁₋₆alkyl;
   (vi) independently selected from the group consisting of —C(O)N(R^{N})— and —N(R^{N})C(O), wherein each R^{N} is independently hydrogen or C₁₋₆alkyl;
L¹ is a bond, C(O), C(S), S(O)₂, P(O)(OH), or P(S)(OH);
R is —O(R^{a}) or —C₁₋₆alkyl-O(R^{a}), wherein R^{a} is hydrogen or a hydroxyl protecting group; and
   R^{T1} is as defined for Formula (X). In some embodiments, -L′-R^{T} is

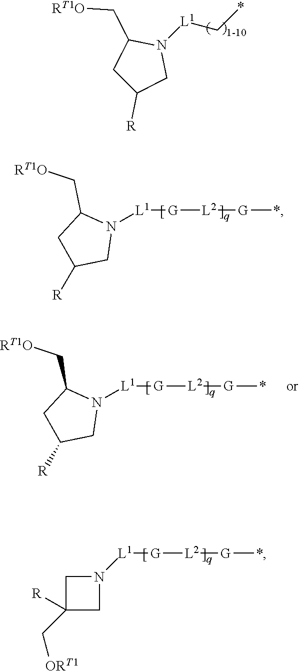

wherein* is the bond to ZZ;
q is 1, 2, 3, 4, 5, 6, 7, 8, 9, or 10 (e.g., an integer from 1 to 8, an integer from 1 to 5, or an integer from 1 to 3);
each L² is
   (i) independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl; or
   (ii) independently selected from the group consisting of —C(O)O—, —OC(O)—, —C(O)N(R^{N})—, —N(R^{N})C(O)—, —OC(O)N(R^{N})—, —N(R^{N})COO, N(R^{N})C(O)N(R^{N})—, —O—, and —N(R^{N})—, wherein each R^{N} is independently hydrogen or C₁₋₆alkyl; or
   (iii) is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl; or
   (iv) independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl; or
   (v) independently selected from the group consisting of —C(O)N(R^{N})—, —N(R^{N})C(O)—, —O—, and —N(R^{N})—, wherein each R^{N} is independently hydrogen or C₁₋₆alkyl;
   (vi) independently selected from the group consisting of —C(O)N(R^{N})— and —N(R^{N})C(O), wherein each R^{N} is independently hydrogen or C₁₋₆alkyl;
L¹ is a bond, C(O), C(S), S(O)₂, P(O)(OH), or P(S)(OH);
R is —O(R^{a}) or —C₁₋₆alkyl-O(R^{a}), wherein R^{a} is hydrogen or a hydroxyl protecting group; and
   R^{T1} is as defined for Formula (X).

In some embodiments, -L′-R^{T} is

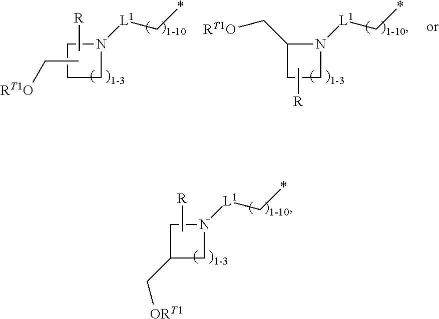

wherein* is the bond to ZZ;
L¹ is a bond, C(O), C(S), S(O)₂, P(O)(OH), or P(S)(OH);
R is —O(R^{a}) or —C₁₋₆alkyl-O(R^{a}), wherein R^{a} is hydrogen or a hydroxyl protecting group; and
R^{T1} is as defined for Formula (X).

### R^{L} Embodiments, Formula (X)

In one embodiment the Formula (X), or a salt thereof, R^{L} is —N(R³)(R⁴), —O(R⁵), —S(R⁵), or —R⁵, wherein R⁵ is according to one Formulae (x-a) through (x-s):

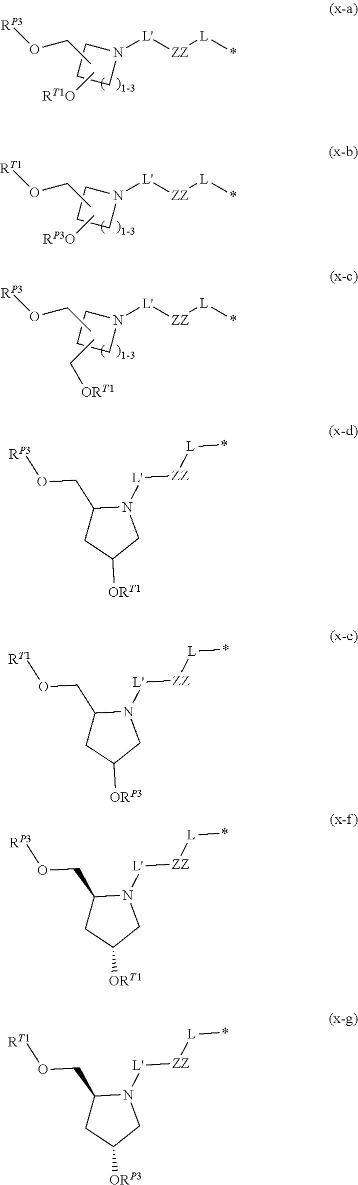

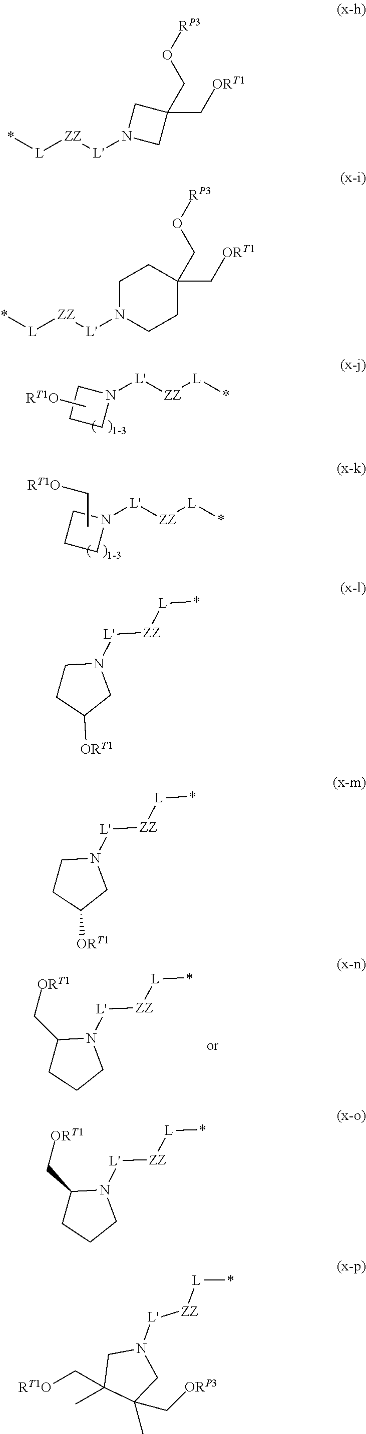

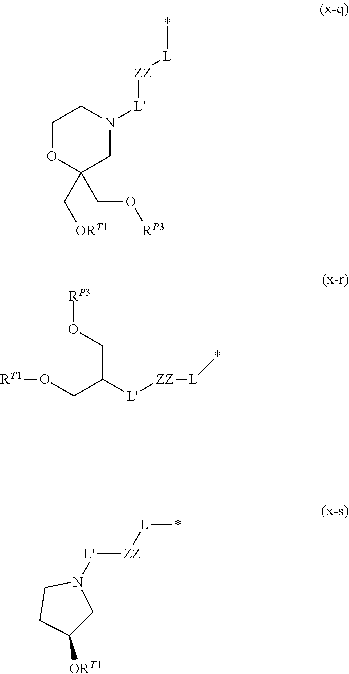

wherein R^{P3} is hydrogen or a hydroxyl protecting group and L, ZZ, L′ and R^{T1} are as defined for Formula (X) or any embodiment herein.

In another embodiment, R^{L} is according to one of Formulae (xi-a) through xi-n):

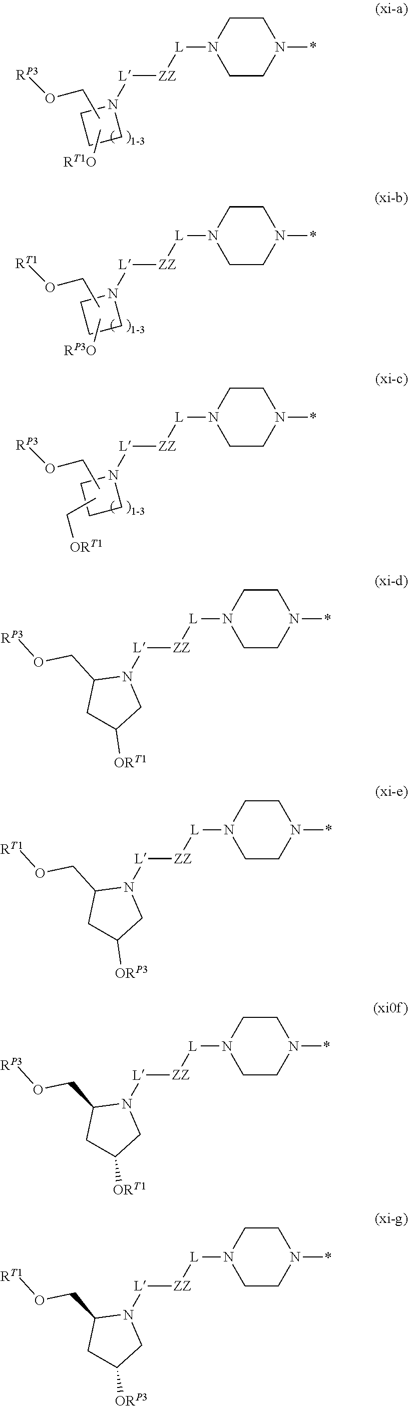

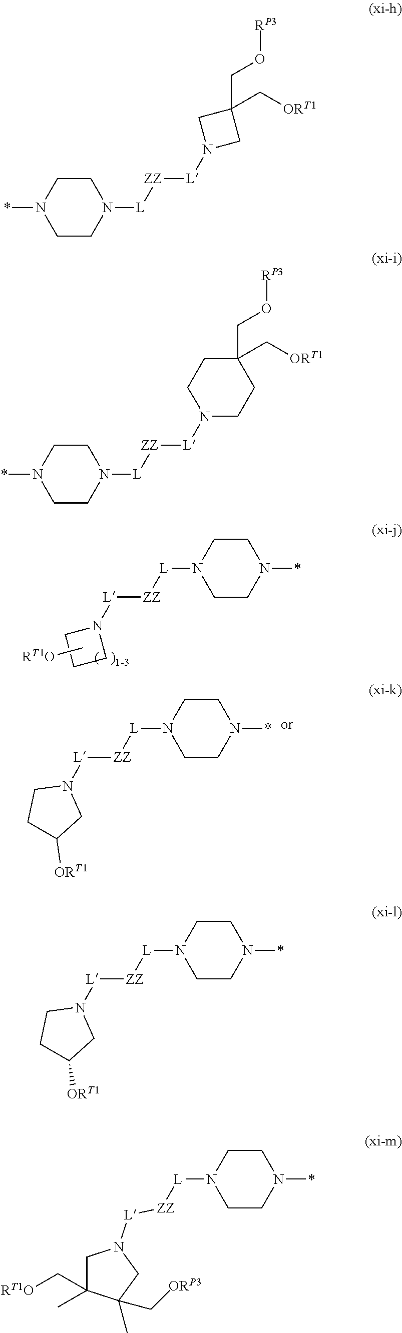

wherein R^{P3} is hydrogen or a hydroxyl protecting group, and L, ZZ, L′ and R^{T1} are as defined for Formula (X) or any embodiment herein.

### L Embodiments, Formula (X)

In some embodiments of any one of Formula (X) and any embodiments thereof, L is -L¹-[G-L²]_{q}-G-L³-*, wherein * is the bond to ZZ.

In another embodiment, wherein L is -L¹-[G-L²]_{q}-G-L³-*, wherein q is 0, 1, 2, 3, 4, or 5.

In another embodiment, L is -L¹-[G-L²]_{q}-G-L³-*, wherein q is 0, 1, 2, 3, or 4

In another embodiment, L is -L¹-[G-L²]_{q}-G-L³-*, wherein q is 0, 1, 2, or 3.

In another embodiment, L is -L¹-[G-L²]_{q}-G-L³-*, wherein q is 0, 1, or 2.

In another embodiment, L is -L¹-[G-L²]_{q}-G-L³-*, wherein q is 1, 2, 3, 4, or 5.

In another embodiment, L is -L¹-[G-L²]_{q}-G-L³-*, wherein q is 1, 2, 3, or 4.

In another embodiment, L is -L¹-[G-L²]_{q}-G-L³-*, wherein q is 1, 2, or 3.

In another embodiment, L is -L¹-[G-L²]_{q}-G-L³-*, wherein q is 1 or 2.

In another embodiment, L is -L¹-[G-L²]_{q}-G-L³-*, wherein q is 4. In another embodiment, L is -L¹-[G-L²]_{q}-G-L³-*, wherein q is 3. In another embodiment, L is -L¹-[G-L²]_{q}-G-L³-*, wherein q is 2.

In another embodiment, L is -L¹-G-L²-G-L³-*. In another embodiment, L is -L¹-G-L³-*. In another embodiment, L is -G-L³-*. In another embodiment, L is -L¹-G-*. In another embodiment, L is -G-*.

In some embodiments, each instance of A-B-A- is independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl.

In some embodiments, each instance of A-B-A- is independently selected from the group consisting of —C(O)O—, —OC(O)—, —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl.

In some embodiments, each instance of A-B-A- is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl.

In some embodiments, each instance of A-B-A- is independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl.

In some embodiments, each instance of A-B-A- is independently selected from the group consisting of —C(O)N(R^{N})—, —N(R^{N})C(O)—, —O—, and —N(R^{N})—, wherein each R^{N} is independently hydrogen or C₁₋₆alkyl.

In some embodiments,
D and F are each independently a bond, C₁₋₁₀alkyl, C₂₋₁₀alkenyl, or C₂₋₁₀alkynyl, each optionally substituted with 1, 2, 3, or 4 R groups; and
E is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments,
D and F are each independently a bond or C₁₋₁₀alkyl optionally substituted with 1, 2, 3, or 4 R groups; and
E is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments, each G is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments, each G is independently C₁₋₁₀alkyl, optionally substituted with 1, 2, or 3 R groups.

In some embodiments, each G is independently C₁₋₁₀alkyl, optionally substituted with 1 or 2 R groups.

In some embodiments, each G is independently C₁₋₁₀alkyl, optionally substituted with one R group

In some embodiments, L is -L1-G-L³-*, wherein * is the bond to ZZ;
G is -D-E-F-, wherein D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-A-;
L³ is a bond or -A-B-A-;
each A is independently a bond, —O—, —S—, or —N(R^{N})—, wherein each R^{N} is independently hydrogen or C₁₋₆alkyl.
each B is independently a bond, CH₂, C(O), S(O)₂, P(O)(OH), or P(S)(OH); and

In some embodiments, L is -L1-G-L³-*, wherein * is the bond to ZZ;
G is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-;
L³ is a bond or -A-B-A-;
each A is independently a bond, —O—, —S—, or —N(R^{N})—, wherein each R^{N} is independently hydrogen or C₁₋₆alkyl; and
each B is independently a bond, CH₂, C(O), S(O)₂, P(O)(OH), or P(S)(OH; and

In some embodiments, L is -L¹-G-*, wherein * is the bond to ZZ;
G is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-A-, wherein,
   A is a bond, —O—, —S—, or —N(R^{N})—, wherein each R^{N} is independently hydrogen or C₁₋₆ alkyl; and
   B is a bond, C(O), C(S), S(O)₂, P(O)(OH), or P(S)(OH).

In some embodiments, L is -L¹-G-*, wherein * is the bond to ZZ;
G is C₁₋₁₀alkyl, C₂₋₁₀alkenyl, C₂₋₁₀alkynyl, each of which is optionally substituted with 1 or 2 R groups;
L¹ is -B-A-, wherein
   A is a bond, —O—, —S—, or —N(R^{N})—, each R^{N} is independently hydrogen or C₁₋₆alkyl; and
   B is a bond, C(O), S(O)₂, P(O)(OH), or P(S)(OH).

In some embodiments L is -L¹-G-*, wherein * is the bond to ZZ;
G is C₁₋₁₀alkyl or C₂₋₁₀alkenyl, each of which is optionally substituted with 1 or 2 R groups;
L¹ is bond, C(O), S(O)₂, P(O)(OH), or P(S)(OH); and
R^{N} is hydrogen or C₁₋₆alkyl.

In some embodiments, L is

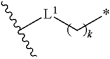

wherein * is the bond to ZZ; k is an integer from 1 to 10; L¹ is bond, C(O), C(S), C(NR^{N}), S(O)₂, P(O)(OH), or P(S)(OH); and R^{N} is hydrogen or C₁₋₆ alkyl.

In some embodiments, L is

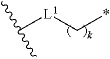

wherein * is the bond to ZZ; k is an integer from 1 to 10; L¹ is bond, C(O), P(O)(OH), or P(S)(OH).

In some embodiments, L is

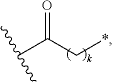

wherein * is the bond to ZZ; k is an integer from 1 to 10; or an integer from 2 to 10; or an integer from 3 to 10; or an integer from 4 to 10; or an integer from 5 to 10; or an integer from 5 to 9; or an integer from 5 to 8; or an integer from 5 to 7.

In some embodiments, L is

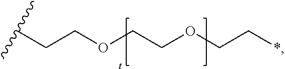

wherein * is the bond to ZZ; t is an integer from 0 to 10 (e.g., an integer from 1 to 5; or 1; or 2; or 3).

In some embodiments, L is

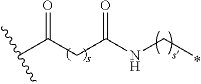

wherein * is the bond to ZZ, t is an integer from 0 to 10 (e.g., an integer from 1 to 5 or 1; or 2; or 3); a is an integer from 1 to 3; and s and s′ are each independently an integer from 1 to 24 (e.g., an integer from 1 to 16; an integer from 1 to 10; an integer from 3 to 10; an integer from 3 to 7; or an integer from 4 to 6).

In some embodiments, L is

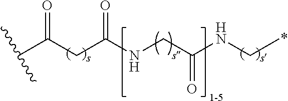

wherein * is the bond to ZZ; a is 1, 2 or 3; and each s, s′, and s″ independently is an integer from 1 to 24 (e.g., an integer from 1 to 16, an integer from 1 to 10, an integer from 3 to 10, an integer from 3 to 7, or an integer from 4 to 6).

In some embodiments, L is

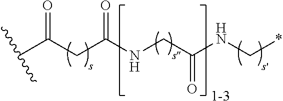

wherein * is the bond to ZZ; and s, s′, and s″ are independently is an integer from 1 to 24 (e.g., an integer from 1 to 16, an integer from 1 to 10, an integer from 3 to 10, an integer from 3 to 7, or an integer from 4 to 6).

In some embodiments, L is

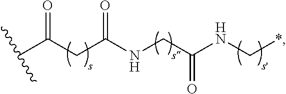

wherein * is the bond to ZZ and each s, s′, and s″ independently is an integer from 1 to 24 (e.g., an integer from 1 to 16, an integer from 1 to 10, an integer from 3 to 10, an integer from 3 to 7, or an integer from 4 to 6)

In some embodiments, L is

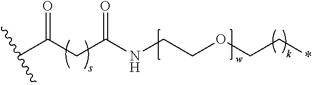

wherein * is the bond to ZZ; s and k are independently is an integer from 1 to 20 (e.g., an integer from 1 to 16, an integer from 1 to 10, an integer from 3 to 10, an integer from 3 to 7 or an integer from 4 to 6); and w is an integer from 1 to 10 (e.g., an integer from 1 to 16, an integer from 1 to 10, an integer from 3 to 10, an integer from 3 to 7 or an integer from 4 to 6).

In some embodiments, L is

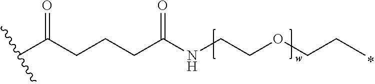

wherein * is the bond to ZZ and w is an integer from 1 to 20 (e.g., an integer from 1 to 16, an integer from 1 to 10, an integer from 3 to 10, an integer from 3 to 7 or an integer from 4 to 6).

### R^{P3} Embodiments, Formula (X)

In embodiments of Formula (X), including embodiments of Formulae (x-a) through (x-s) and (xi-a) through (xi-m), R^{P3}, when present, is hydrogen.

In another embodiment, R^{P3}, when present, is a hydroxyl protecting group.

In another embodiment, R^{P3}, when present, is a hydroxyl protecting group selected from the group consisting of acetyl, trifluoroacetyl, trichloroacetyl, pivaloyl, t-butyl, allyl, optionally substituted benzyl (such as benzyl, 2-nitrobenzyl, 4-nitrobenzyl, 2,6-dichlorobenzyl, 4-chlorobenzyl, 4-fluorobenzyl, 4-bromobenzyl, 4-methoxybenzyl, 3,4-dimethoxybenzyl, 2-cyanobenzyl, 4-cyanobenzyl, 4-phenylbenzyl), 2-picolyl, and 4-picolyl.

In another embodiment, R^{P3}, when present, is a hydroxyl protecting group selected from the group consisting of methoxymethyl (MOM), methylthiomethyl (MTM), ethoxymethyl, 2-methoxyethoxymethyl (MEM), 2,2,2-trichloroethoxymethyl, t-butoxymethyl, benzyloxymethyl (BOM), 4-methoxybenzyloxymethyl (Mbom), (phenyldimethylsilyl)methoxymethyl (SMOM), 2-(Trimethylsilyl)ethoxymethyl (SEM), and t-butylthiomethyl,

In another embodiment, R^{P3}, when present, is a hydroxyl protecting group selected from the group consisting of 2-tetrahydropyranyl (THP), 4-methoxytetrahydropyran-2-yl (MTHP), 4-methoxytetrahydrothiopyran-2-yl, 3-bromotetrahydropyran-2-yl, and 2-tetrahydrothiopyranyl.

In another embodiment, R^{P3}, when present, is a hydroxyl protecting group selected from the group consisting of trimethylsilyl (TMS), triethylsilyl (TES), triisopropylsilyl (TIPS), t-butyldimethylsilyl (TBDMS), t-butyldiphenylsilyl (TBDPS), Isopropyldimethylsilyl (IPDMS), and diethylisopropylsilyl (DEIPS).

In another embodiment, R^{P3}, when present, is a hydroxyl protecting group selected from the group consisting of diphenylmethyl, 9-phenylxanthine-9-yl (Pixyl), 9-(p-methoxyphenyl)xanthine-9-yl (MOX), and optionally substituted trityl (e.g., trityl (Trt), 2-chlorotrityl (Clt), 4′-methoxytrityl (Mmt), 4′-methyltrityl (Mtt), 4,4′-dimethoxytrityl (DMT)), and 4,4′,4″-trimethoxytrityl.

In another embodiment, R^{P3}, when present, is an optionally substituted trityl group (e.g., trityl (Trt), 2-chlorotrityl (Clt), 4-methoxytrityl (Mint), 4-methyltrityl (Mtt), 4,4′-dimethoxytrityl (DMT)), or 4,4′,4″-trimethoxytrityl).

In another embodiment, R^{P3}, when present, is a 4,4′-dimethoxytrityl (DMT) group.

In another embodiment, the compound of Formula (X) is according to one of Formulae (X-b) through (X-e) and (X-x):

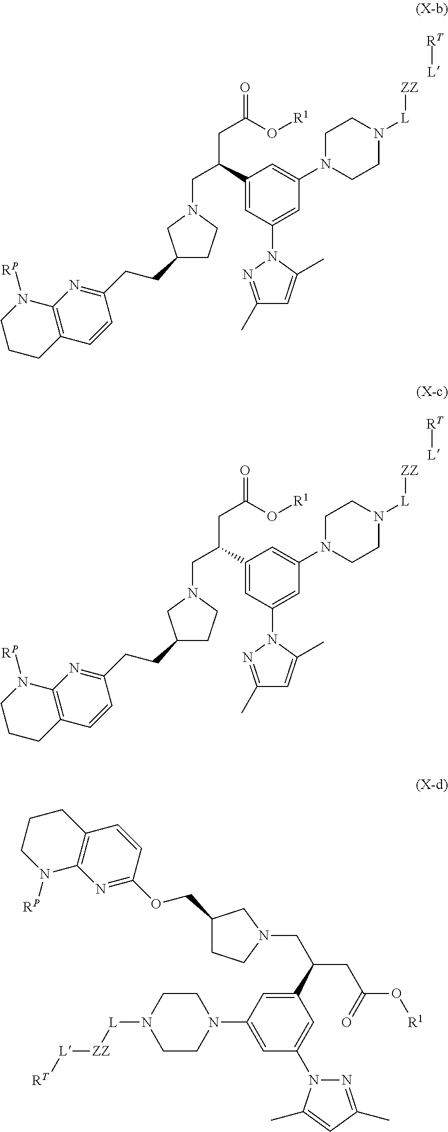

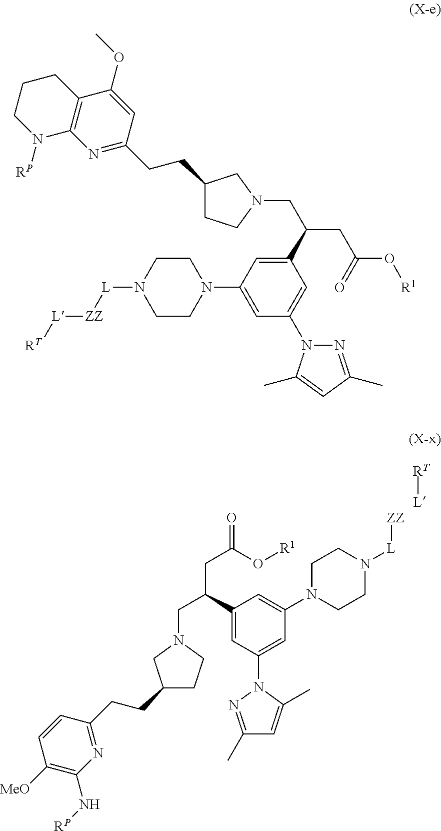

or a salt thereof, wherein R^{P} is hydrogen or a nitrogen protecting group (e.g., a nitrogen protecting group), wherein R and the remaining variables are as defined in Formula (X). In one embodiment of Formulae (X-b) through (X-e) and (X-x), R¹ is hydrogen. In another embodiment of Formulae (X-b) through (X-e) and (X-x), R¹ is C₁₋₆alkyl (e.g., methyl or t-butyl). In another embodiment of Formulae (X-b) through (X-e) and (X-x), R¹ is hydrogen and R^{P} is hydrogen. In another embodiment of Formulae (X-b) through (X-e) and (X-x), R¹ is hydrogen and a nitrogen protecting group. In another embodiment of Formulae (X-b) through (X-e) and (X-x), R¹ is C₁₋₆alkyl (e.g., methyl or t-butyl) and R^{P} is hydrogen. In another embodiment of Formulae (X-b) through (X-e) and (X-x), R¹ is C₁₋₆alkyl (e.g., methyl or t-butyl) and a nitrogen protecting group.

### -L-ZZ-L′-R^{T} Embodiments

In an embodiment, of any one of Formula (X) and Formulae (X-a) through (X-e) and (X-x), -L-ZZ-L′-R^{T} is

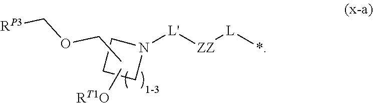

In an embodiment, of any one of Formula (X) and Formulae (X-a) through (X-e) and (X-x), -L-ZZ-L′-R^{T} is

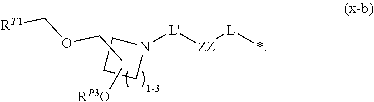

In an embodiment, of any one of Formula (X) and Formulae (X-a) through (X-e) and (X-x), -L-ZZ-L′-R^{T} is

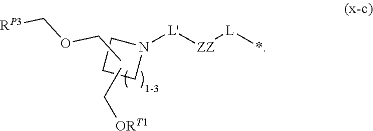

In an embodiment, of any one of Formula (X) and Formulae (X-a) through (X-e) and (X-x), -L-ZZ-L′-R^{T} is

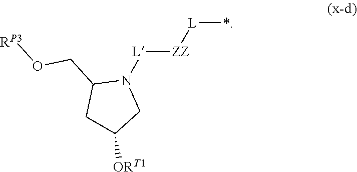

In an embodiment, of any one of Formula (X) and Formulae (X-a) through (X-e) and (X-x), -L-ZZ-L′-R^{T} is

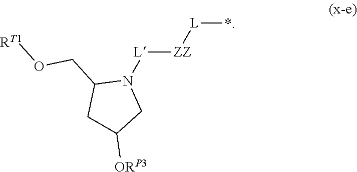

In an embodiment, of any one of Formula (X) and Formulae (X-a) through (X-e) and (X-x), -L-ZZ-L′-R^{T} is

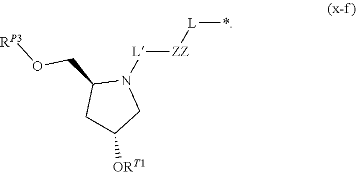

In an embodiment, of any one of Formula (X) and Formulae (X-a) through (X-e) and (X-x), -L-ZZ-L′-R^{T} is

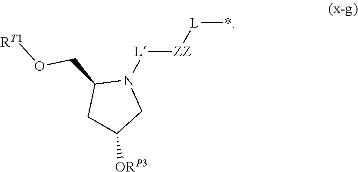

In an embodiment, of any one of Formula (X) and Formulae (X-a) through (X-e) and (X-x), -L-ZZ-L′-R^{T} is

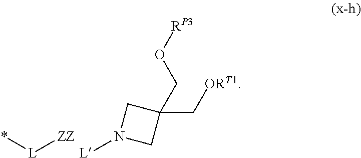

In an embodiment, of any one of Formula (X) and Formulae (X-a) through (X-e) and (X-x), -L-ZZ-L′-R^{T} is

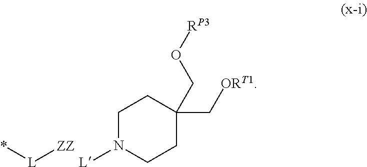

In an embodiment, of any one of Formula (X) and Formulae (X-a) through (X-e) and (X-x), -L-ZZ-L′-R^{T} is

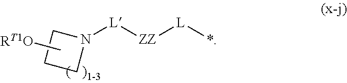

In an embodiment, of any one of Formula (X) and Formulae (X-a) through (X-e) and (X-x), -L-ZZ-L′-R^{T} is

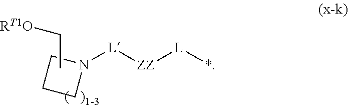

In an embodiment, of any one of Formula (X) and Formulae (X-a) through (X-e) and (X-x), -L-ZZ-L′-R^{T} is

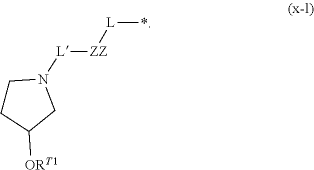

In an embodiment, of any one of Formula (X) and Formulae (X-a) through (X-e) and (X-x), -L-ZZ-L′-R^{T} is

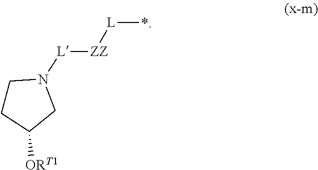

In an embodiment, of any one of Formula (X) and Formulae (X-a) through (X-e) and (X-x), -L-ZZ-L′-R^{T} is

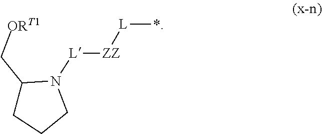

In an embodiment, of any one of Formula (X) and Formulae (X-a) through (X-e) and (X-x), -L-ZZ-L′-R^{T} is

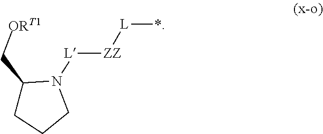

In an embodiment, of any one of Formula (X) and Formulae (X-a) through (X-e) and (X-x), -L-ZZ-L′-R^{T} is

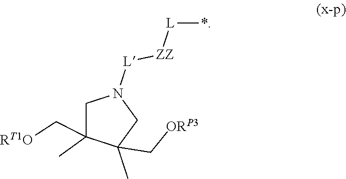

In an embodiment, of any one of Formula (X) and Formulae (X-a) through (X-e) and (X-x), -L-ZZ-L′-R^{T} is

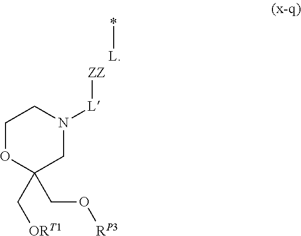

In an embodiment, of any one of Formula (X) and Formulae (X-a) through (X-e) and (X-x), -L-ZZ-L′-R^{T} is

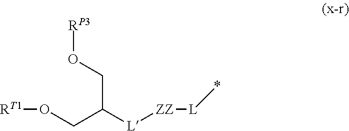

In an embodiment, of any one of Formula (X) and Formulae (X-a) through (X-e) and (X-x), -L-ZZ-L′-R^{T} is

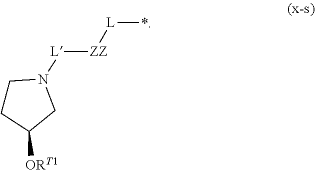

### R^{T1} Embodiments, Formula (X)

### Nucleotide CONJUGATES

In embodiments of any embodiments of R^{L} of Formula (X), Formula (X-a) through (X-e) and (X-x), Formula (x-a) through (x-s), and Formula (xi-a) through (xi-m),
R^{T1} is -L^{L}-oligonucleotide, wherein L^{L} is a divalent linker that connects to the 3′-end of the oligonucleotide, the 5′-end of the oligonucleotide, or an internal 2′- or 3′ position on a internal nucleotide (i.e., a nucleotide that is not the 5′-terminal or 3′-terminal nucleoside).

In some embodiments, R^{T1} is -L^{L}-oligonucleotide, wherein L^{L} is a divalent linker that connects to the 3′-end of the oligonucleotide, such as one of:
directly to the 3′-carbon of the 3′-terminal nucleoside;
directly to the 3′-O of the 3′-terminal nucleoside;
directly to the 4′-carbon of the 3′-terminal nucleoside;
directly to the 2′-carbon of the 3′-terminal nucleoside; or
directly to the 2′-O of the 3′-terminal nucleoside.

In some embodiments, R^{T1} is -L^{L}-oligonucleotide, wherein L^{L} is a divalent linker that connects to the 5′-end of the oligonucleotide, such as one of:
directly to the 5′-carbon of the 5′-terminal nucleoside;
directly to the 5′-O of the 5′-terminal nucleoside;
directly to the 4′-carbon of the 5′-terminal nucleoside;
directly to the 2′-carbon of the 5′-terminal nucleoside; or
directly to the 2′-O of the 5′-terminal nucleoside.

In some embodiments, R^{T1} is -L^{L}-oligonucleotide, wherein L^{L} is a divalent linker that connects to an internal 2′- or 3′ position on an internal nucleotide.

In some embodiments, R^{T1} is -L^{L}-oligonucleotide, wherein L^{L} is a divalent linker that connects to an internal 2′-position on a internal nucleotide.

In some embodiments, R^{T} is R^{T1}, wherein R^{T1} is -L^{L}-oligonucleotide, wherein L^{L} is a divalent linker that connects to an internucleotide linkage (i.e., to an oxygen atom in a phosphodiester linkage to form a phosphotriester; or to a nitrogen when the internucleotide linkage is a phosphoroamidate).

In another embodiment of any of the preceding embodiments of R^{T1}, when L^{L} connects to a carbon atom on a nucleoside, then L^{L} is -B³-A³-, wherein
B³ is —P(O)(OH)—, —P(S)(OH)—, or —P(S)(SH)—; and
A³ is —O—, —S—, or —N(H)—.

In another embodiment, when L^{L} connects to a carbon atom on a nucleoside, then L^{L} is -B³-A³-, wherein
B³ is —P(O)(OH)— or —P(S)(OH)—; and
A³ is —O—.

In another embodiment, when L^{L} connects to a oxygen atom on a nucleoside, then L^{L} is —P(O)(OH)—, —P(S)(OH)—, or —P(S)(SH).

In another embodiment, when L^{L} connects to a oxygen atom on a nucleoside, then L^{L} is —P(O)(OH)— or —P(S)(OH)—.

In another embodiment, when L^{L} connects to a oxygen atom on a nucleoside, then L^{L} is —P(O)(OH)—.

In another embodiment, when L^{L} connects to a oxygen atom on a nucleoside, then L^{L} is —P(S)(OH)—.

In another embodiment, when L^{L} connects to a oxygen atom on a nucleoside, then
L^{L} is -B³-A³-L^{L1}-A³-B³-, wherein
   B³ is a bond, —C(O)—, C(S)—, C(NH), S(O), S(O)₂, —P(O)(OH)—, —P(S)(OH)—, or —P(S)(SH);
   A³ is a bond, —O—, —S—, or —N(H)—; and
   L^{L1} is C₁₋₁₀alkyl, C₂₋₁₀alkenyl, C₂₋₁₀alkynyl, C₃₋₁₀cycloalkyl, 3-10 membered heterocyclyl, aryl, or heteroaryl.

In another embodiment, when L^{L} connects to a oxygen atom on a nucleoside, then L^{L} is -B³-L^{L1}-B³—, wherein B³ is —C(O)—; and L^{L1} is C₁₋₁₀alkyl.

In another embodiment, when L^{L} connects to a oxygen atom on a nucleoside, then L^{L} is a bond.

In certain embodiments, R^{T} is R^{T1} wherein R^{T1} is -L^{L}-oligonucleotide, when L^{L} connects to a oxygen atom on a nucleoside, and L L⁵ is a bond, the nucleoside is of Formula (X-f),

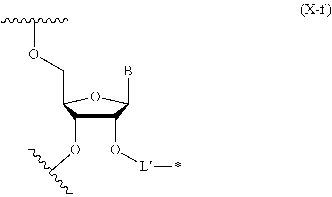

wherein B is an optionally modified nucleobase (e.g., adenine, cytosine, uracil, guanine, 5-methylcytosine, or -5-methyluracil); L¹ is according any of the preceding embodiments; and * represent the bond to ZZ.

In some embodiments of Formula (X-f), -L′- is -L′-G-L³-*, wherein * is the bond to ZZ.

In some embodiments, -L′- is —C₂₋₃₀alkyl-*, wherein the alkyl is optionally substituted with one or two R groups, and * is the bond to ZZ. In some embodiments, -L′- is —C₂₋₃₀alkyl-*, wherein the alkyl is optionally substituted with one or two groups selected from the group consisting of halogen, hydroxy, C₁₋₆alkoxy, amino, C₁₋₆alkylamino, di(C₁₋₆alkylamino), cyano, carboxy, and * is the bond to ZZ. In some embodiments, -L′- is —C₂₋₃₀alkyl-*, wherein the alkyl is optionally substituted with one group selected from the group consisting of halogen, hydroxy, C₁₋₆alkoxy, amino, C₁₋₆alkylamino, di(C₁₋₆ alkylamino), cyano, carboxy, and * is the bond to ZZ. In some embodiments, -L′- is —C₂₋₃₀alkyl-*, wherein the alkyl is optionally substituted with one group selected from the group consisting of hydroxy, amino, and carboxy, and * is the bond to ZZ. In some embodiments, -L′- is —C₂₋₃₀alkyl-*, wherein the alkyl is optionally substituted with hydroxy, and * is the bond to ZZ.

In some embodiments, -L′- is —C₂₋₃₀alkyl-*, wherein * is the bond to ZZ. In some embodiments, -L′- is —C₂₋₁₆alkyl-*, wherein * is the bond to ZZ. In some embodiments, -L′- is —C₄₋₁₂alkyl-*, wherein
* is the bond to ZZ. In some embodiments, -L′- is —C₄₋₁₀alkyl-*, wherein * is the bond to ZZ. In some embodiments, -L′- is —C₅₋₁₀alkyl-*, wherein * is the bond to ZZ. In some embodiments, -L′- is —C₆alkyl-*, wherein * is the bond to ZZ. In some embodiments, -L′- is —C₈alkyl-*, wherein * is the bond to ZZ. In some embodiments, -L′- is —C₁₀alkyl-*, wherein * is the bond to ZZ.

In some embodiments of Formula (X-f), -L′- is -L¹-[G-L²]_{q}-G-L³-*, wherein
* is the bond to ZZ;
q is 0, 1, 2, 3, 4, or 5;
L¹ is a bond or -B-A-;
each L² is independently -A-B-A-;
L³ is a bond or -A-B-A-;
each A is independently a bond, —O—, —S—, or —N(R^{N})—;
each B is independently a bond, CH₂, C(O), C(S), C(NR^{N}), S(O)₂, P(O)(OH), or P(S)(OH);
each R^{N} is independently hydrogen or C₁₋₆alkyl; and
each G is independently C₁₋₁₀alkyl, C₂₋₁₀alkenyl, C₂₋₁₀alkynyl, each of which is optionally substituted with 1, 2, 3, or 4 R groups.

In some embodiments, -L′- is -L¹-[G-L²]_{q}-G-*, wherein * is the bond to ZZ, q is 0, 1, 2, or 3; L¹ is a bond or -B-A-; and
(a) each L² is independently a bond, C(O)O, OC(O), C(O)(NR^{N}), N(R^{N})C(O), SO₂N(R^{N}), N(R^{N})SO₂, OP(O)(OH), OP(S)(OH), P(O)(OH)O, P(S)(OH)O, OP(O)(OH)O, or OP(S)(OH)O, wherein each R^{N} is independently hydrogen or C₁₋₆alkyl; each G is independently C₁₋₁₀alkyl, C₂₋₁₀alkenyl, C₂₋₁₀alkynyl, each of which is optionally substituted with 1 or 2 R groups;
or
(b) each L² is independently a bond, C(O)O, OC(O), C(O)(NR^{N}), N(R^{N})C(O), OP(O)(OH)O, or OP(S)(OH)O, wherein each R^{N} is independently hydrogen or C₁₋₆alkyl; and each G is independently C₁₋₁₀alkyl or C₂₋₁₀alkenyl, each of which is optionally substituted with 1 or 2 R groups.

In some embodiments, -L′- is -[G-L²]_{q}-G-*, wherein * is the bond to ZZ, q is 0, 1, 2, or 3 (e.g., q is 0, 1, or 2; or 0 or 1; or 0; or 1; or 2); each G is independently C₁₋₁₀alkyl, each of which is optionally substituted with 1 or 2 R groups. and
(a) each L² is independently C(O)O or OC(O);
(b) each L² is independently C(O)(NR^{N}) or N(R^{N})C(O), wherein each R^{N} is independently hydrogen or C₁₋₆alkyl
(c) each L² is independently OP(O)(OH)O, or OP(S)(OH)O (e.g., each is OP(O)(OH)O);
or
(d) each L² is a bond.

In some embodiments, the compound of Formula (X) is according to one of Formulae (X-g) through (X-q):

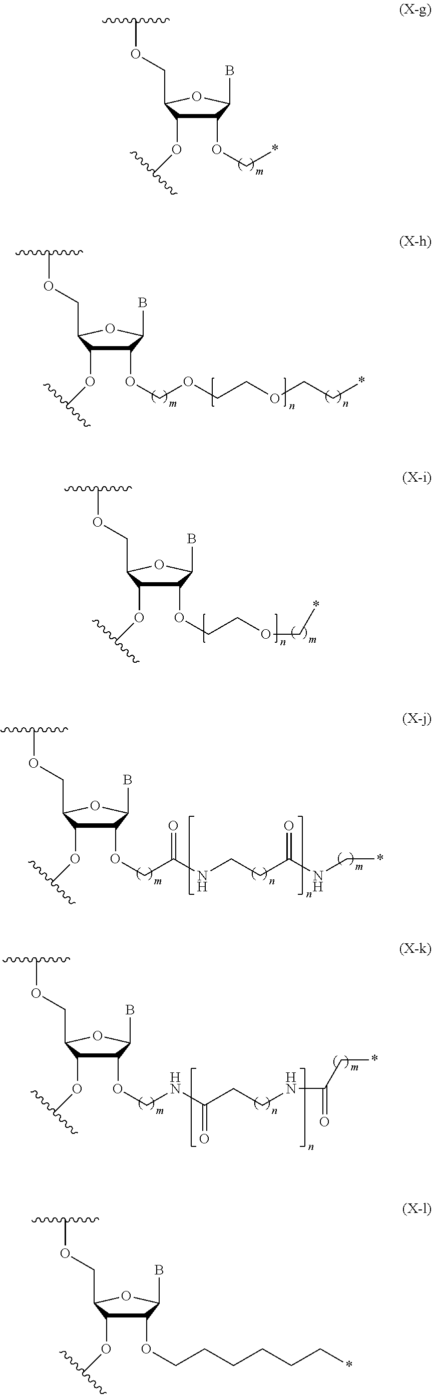

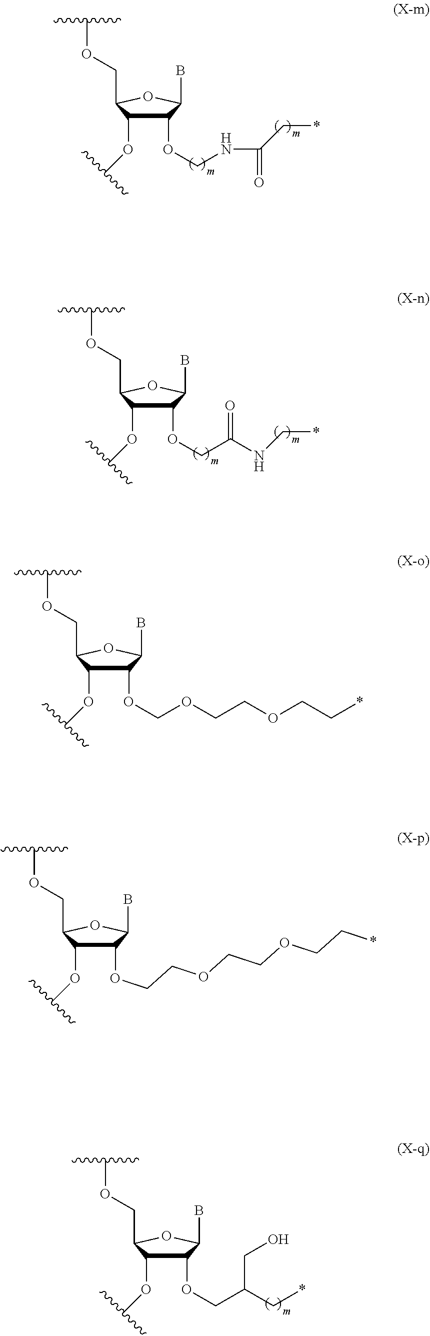

wherein
B is an optionally modified nucleobase (e.g., adenine, cytosine, uracil, guanine, 5-methylcytosine, or -5-methyluracil);
each n is independently 0 or an integer selected from 1-10; (e.g., 1-5, or 1-3, or 3, or 2, or 1);
each m is independently integer selected from 1-20 (e.g., 2-12, or 2-10; or 2-6; or 2; or 3; or 4; or 5; or 6).

In some embodiments, the compound of Formula (X) is according to one of Formulae (X-r) through (X-w):

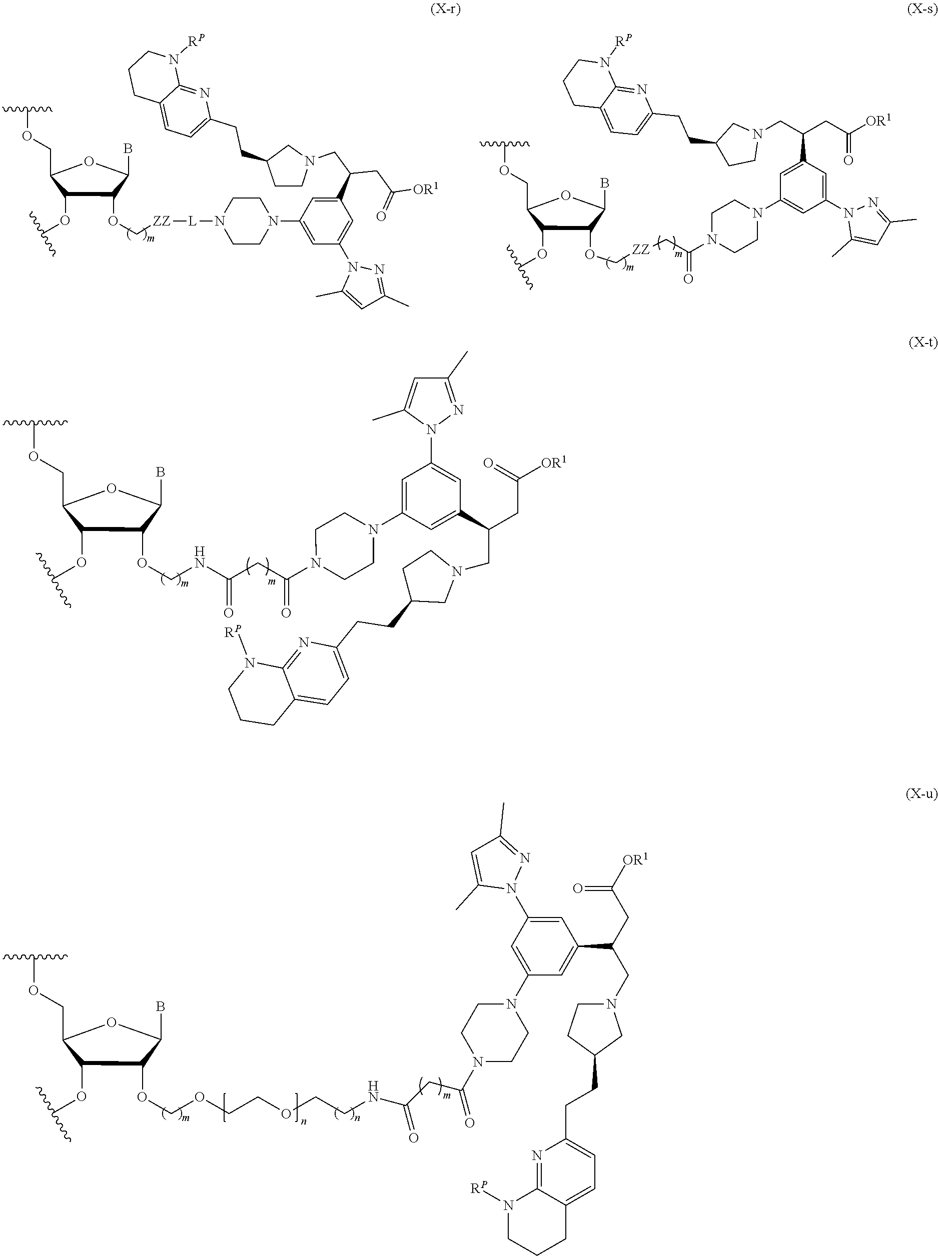

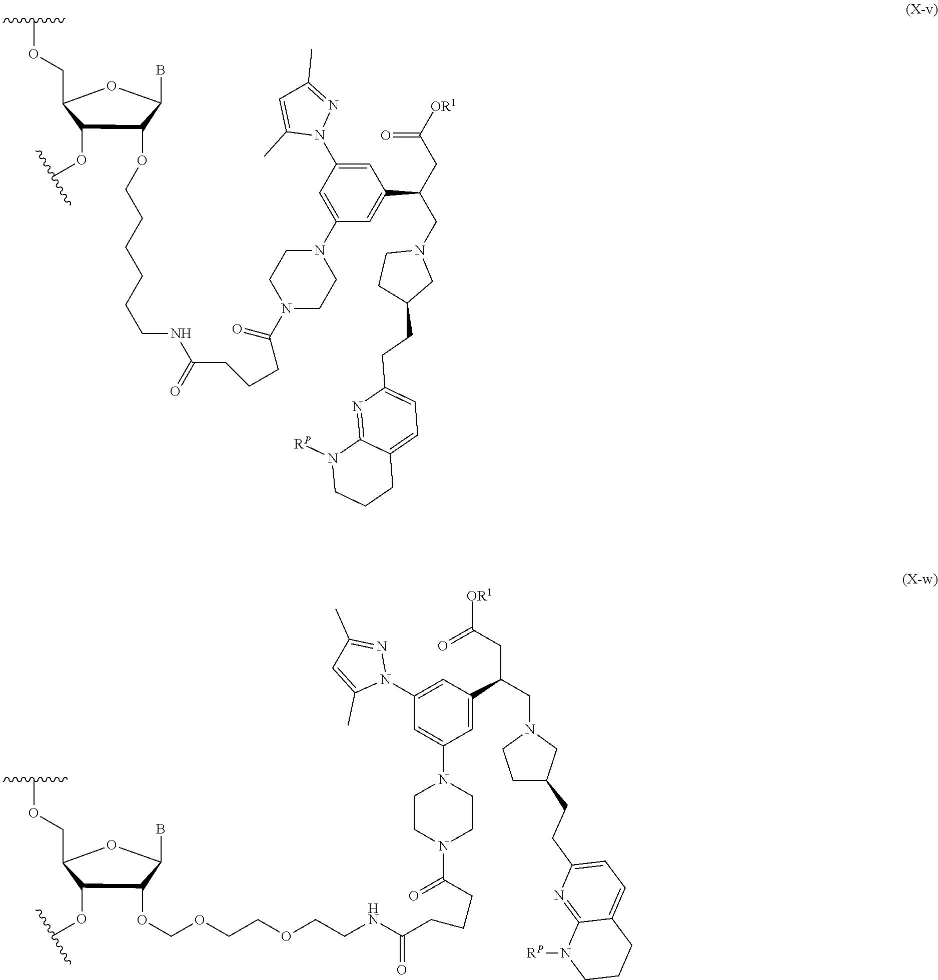

or a salt thereof, wherein
L and ZZ are as defined in Formula (X) or in any embodiment preceding or below;
B is an optionally modified nucleobase (e.g., adenine, cytosine, uracil, guanine, 5-methylcytosine, or -5-methyluracil);
each m is independently integer selected from 1-20 (e.g., 2-12, or 2-10; or 2-6; or 2; or 3; or 4; or 5; or 6);
R^{P} is hydrogen or a nitrogen protecting group (e.g., a nitrogen protecting group); and
R¹ is hydrogen or C₁₋₆alkyl (e.g., methyl ort-butyl).

In one embodiment of Formulae (X-r) through (X-w), R¹ is hydrogen. In another embodiment of Formulae (X-r) through (X-w), R¹ is C₁₋₆alkyl (e.g., methyl ort-butyl). In another embodiment of Formulae (X-r) through (X-w), R¹ is hydrogen and R^{P} is hydrogen. In another embodiment of Formulae (X-r) through (X-w), R¹ is hydrogen and a nitrogen protecting group. In another embodiment of Formulae (X-r) through (X-w), R¹ is C₁₋₆alkyl (e.g., methyl or t-butyl) and R^{P} is hydrogen. In another embodiment of Formulae (X-r) through (X-w), R¹ is C₁₋₆alkyl (e.g., methyl or t-butyl) and a nitrogen protecting group.

In some embodiments, two adjacent nucleosides in the oligonucleotide have one of the formula

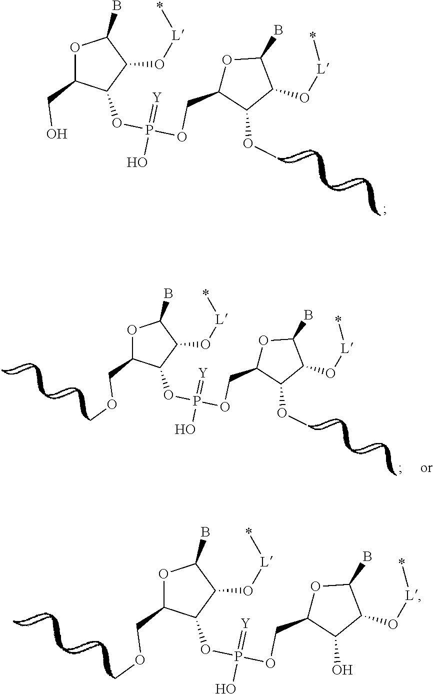

wherein Y is O or S (or O; or S); [symbol] represents the remainder for the oligonucleotide, and B is an optionally modified nucleobase. Therein, each nucleoside of the two adjacent nucleosides is independently according to any one of Formula (X-f) through (X-q). In certain embodiments, each nucleoside is according to the same Formula.

In some embodiments, three adjacent nucleosides in the oligonucleotide have the formula

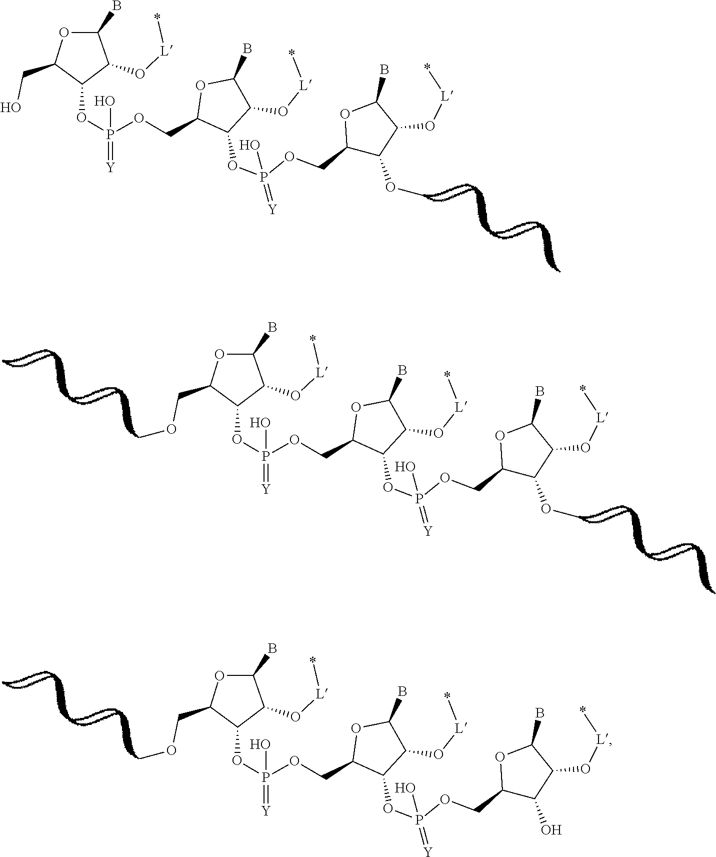

wherein each Y is independently O or S (or O; or S; or O then S or S then 0, 5′ followed by 3′); [symbol] represents the remainder for the oligonucleotide, and B is an optionally modified nucleobase; e.g., each Y is O. Therein, each nucleoside of the three adjacent nucleosides is independently according to any one of Formula (X-f) through (X-q). In certain embodiments, each nucleoside is according to the same Formula.

In some embodiments, four adjacent nucleosides in the oligonucleotide have the formula,

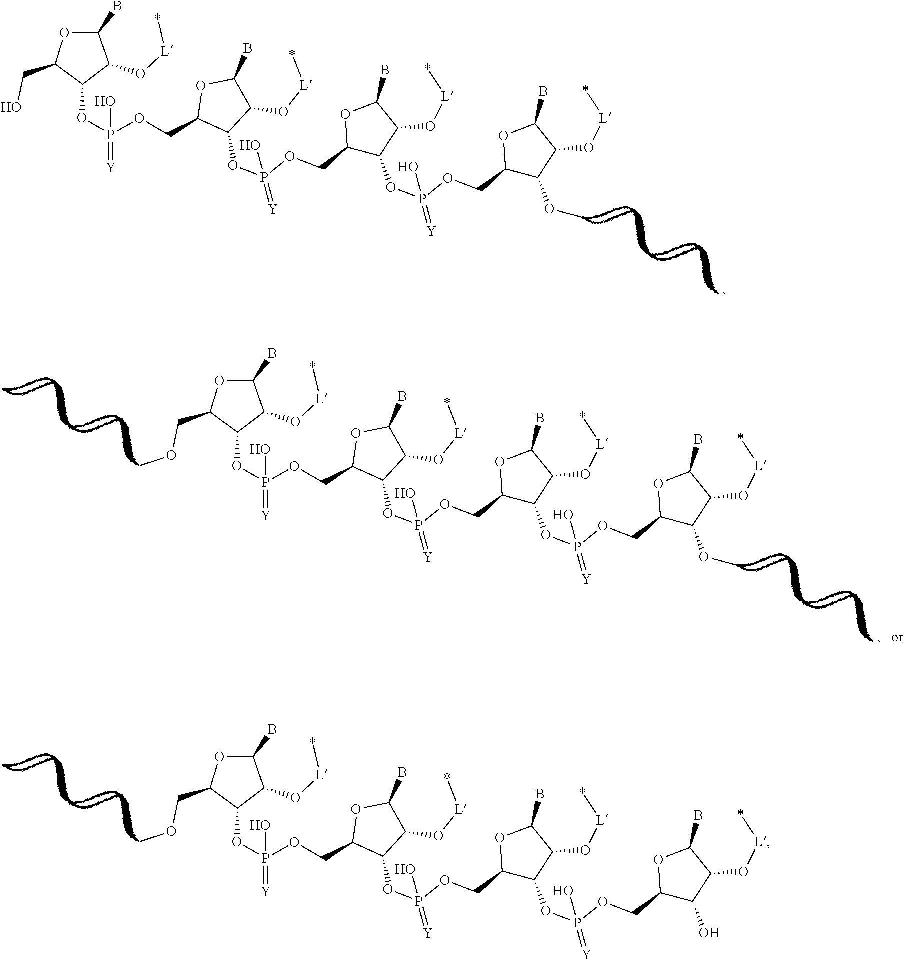

wherein each Y is independently O or S; [symbol] represents the remainder for the oligonucleotide, and B is an optionally modified nucleobase; e.g., each Y is O. Therein, each nucleoside of the four adjacent nucleosides is independently according to any one of Formula (X-f) through (X-q). In certain embodiments, each nucleoside is according to the same Formula. In certain embodiments, each nucleoside is according to the same Formula (X-u) or (X-w).

In certain embodiments, where R^{T} is R^{T1}, wherein R^{T1} is -L^{L}-oligonucleotide, and when L^{L} connects to an oxygen atom or nitrogen atom in an internucleotide linkage, the internucleotide linkage can be of the formula, including the 3′ and 5′ oxygen atoms of the preceding and following nucleosides, respectively,

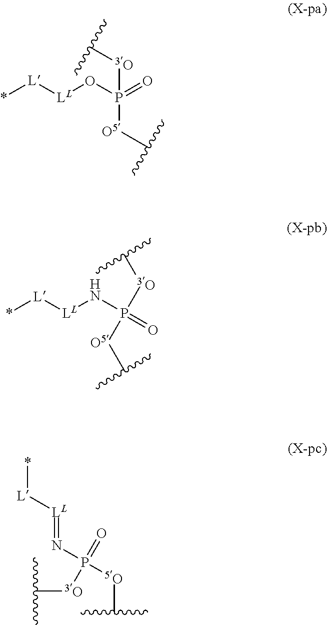

wherein L′ can be, for example a bond, —S(O)₂— or, in for Formula (X-pc), a 5-8 membered heterocyclyl ring optionally substituted with 1 or 2 R groups, as defined herein; and * represent the bond to ZZ.

For example, the preceding includes,

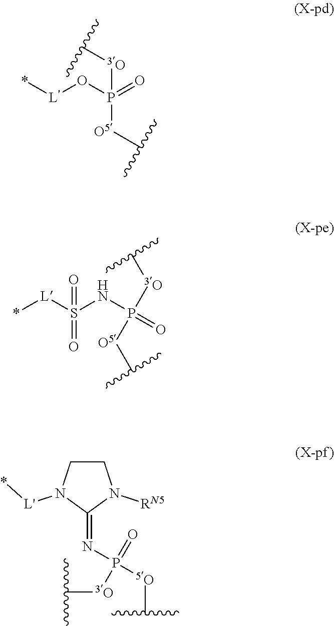

wherein * represent the bond to ZZ; and R^{N5} is hydrogen or C₁₋₁₀ alkyl.

For example, the preceding includes,

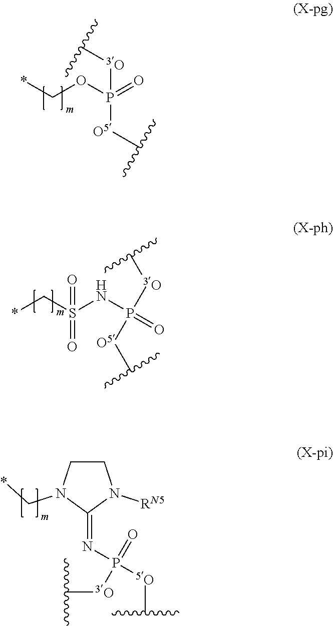

wherein * represent the bond to ZZ; m is an integer selected from 1-20 (e.g., 1-10, or 2-20, or 2-10, or 4-10, or 4-8; or 6-12; or 5; or 6; or 7; or 8; or 9; or 10), and R^{N5} is hydrogen or C₁₋₁₀ alkyl.

In another embodiment, the compound of Formula (X-pd) is

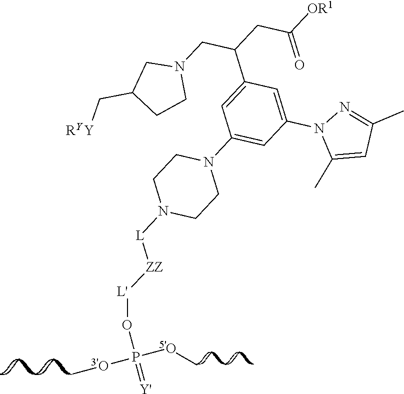

wherein Y′ is O or S, and R^{Y}, Y, R¹, L, L′, and ZZ are as defined for Formula (X). In one embodiment, R¹ is hydrogen. In another embodiment, R¹ is C₁₋₆alkyl (e.g., methyl or t-butyl).

In another embodiment, the compound is

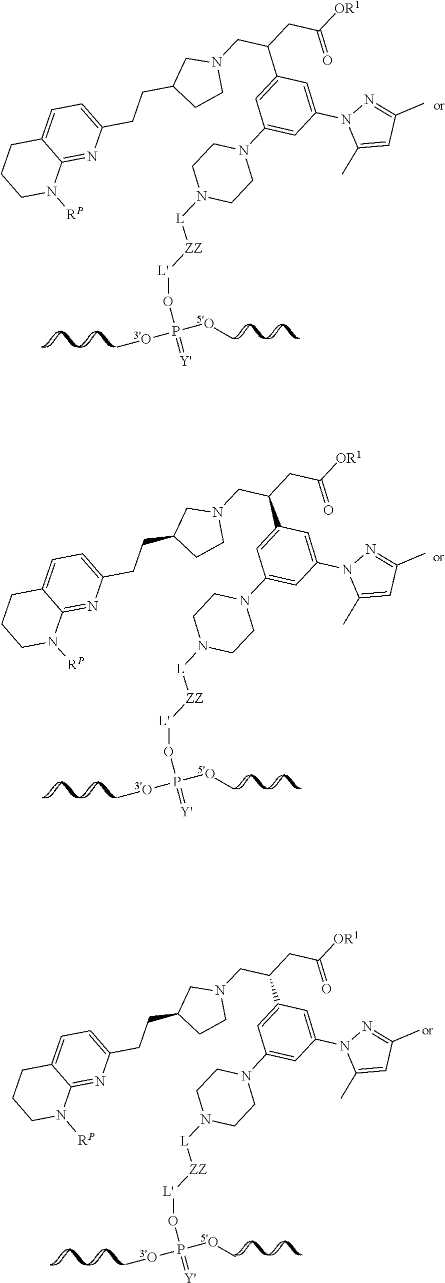

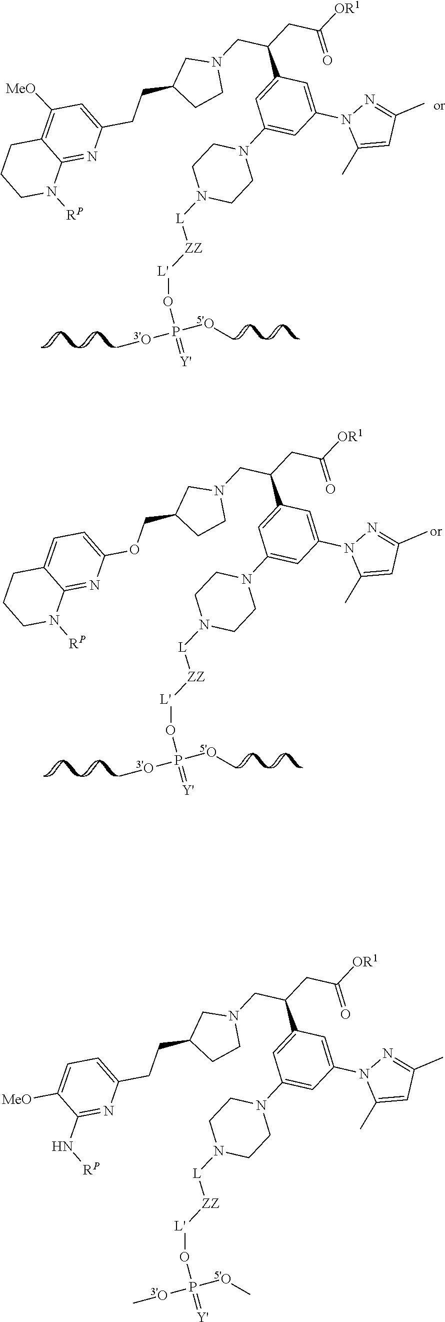

wherein Y′ is O or S, and R¹, L, L′, and ZZ are as defined for Formula (X).

In another embodiment, the compound is

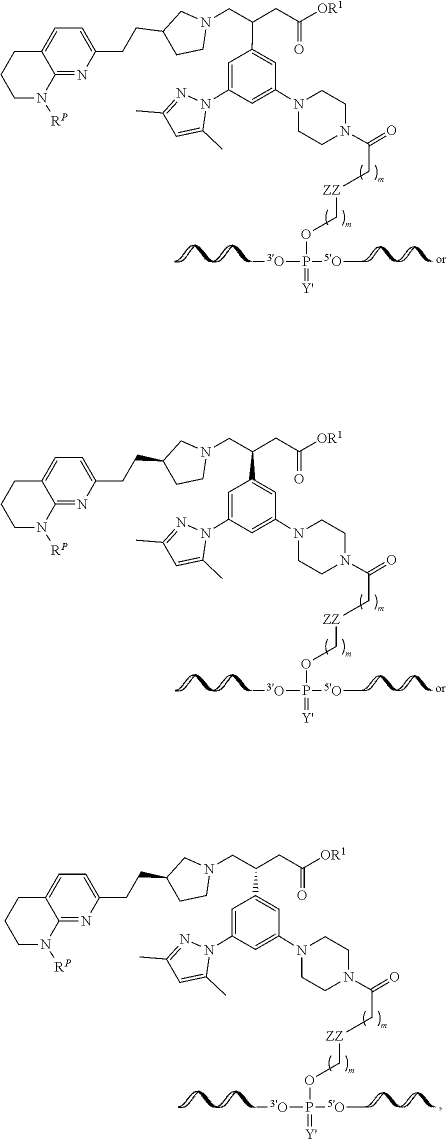

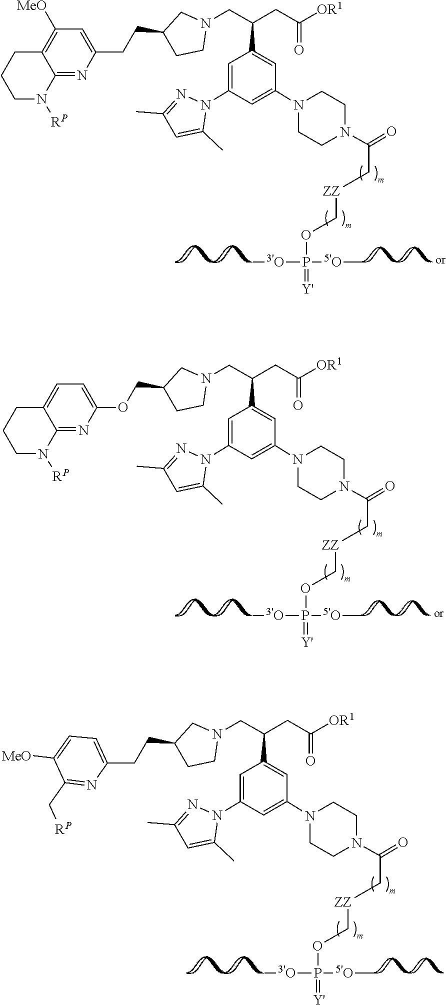

wherein each m is an integer selected from 1-20 (e.g., 2-20, 2-10, 1-10, 2-16, 4-16, 4-8, or 6-12), Y′ is O or S, and R¹, L, L′, and ZZ are as defined for Formula (X).

In another embodiment, the compound of Formula (X-pe) is

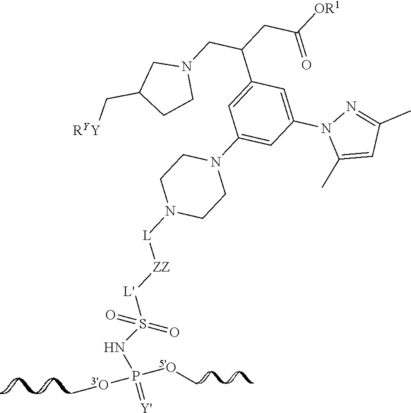

wherein Y′ is O or S, and R^{Y}, Y, R¹, L, L′, and ZZ are as defined for Formula (X). In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound is

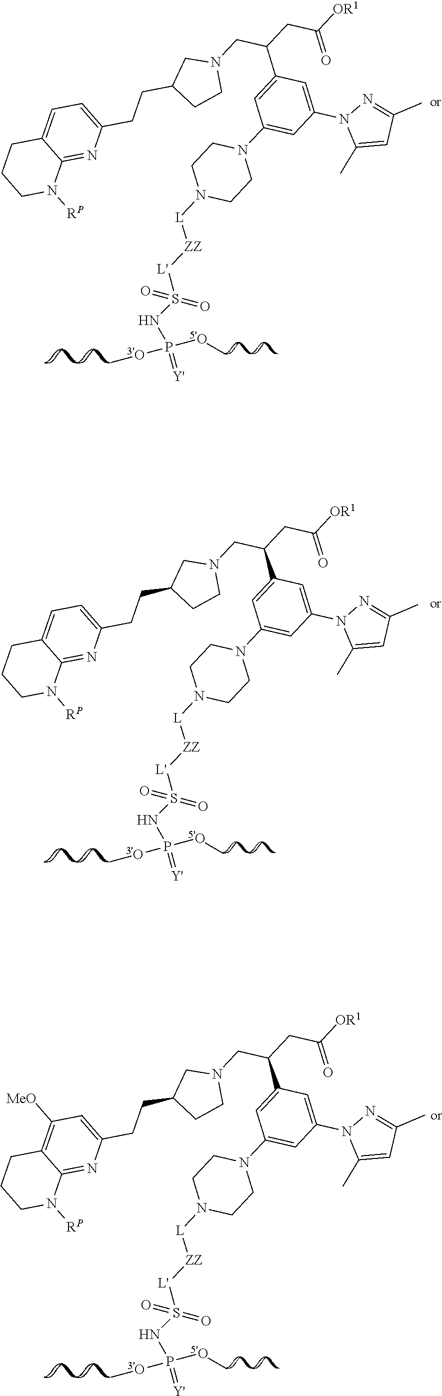

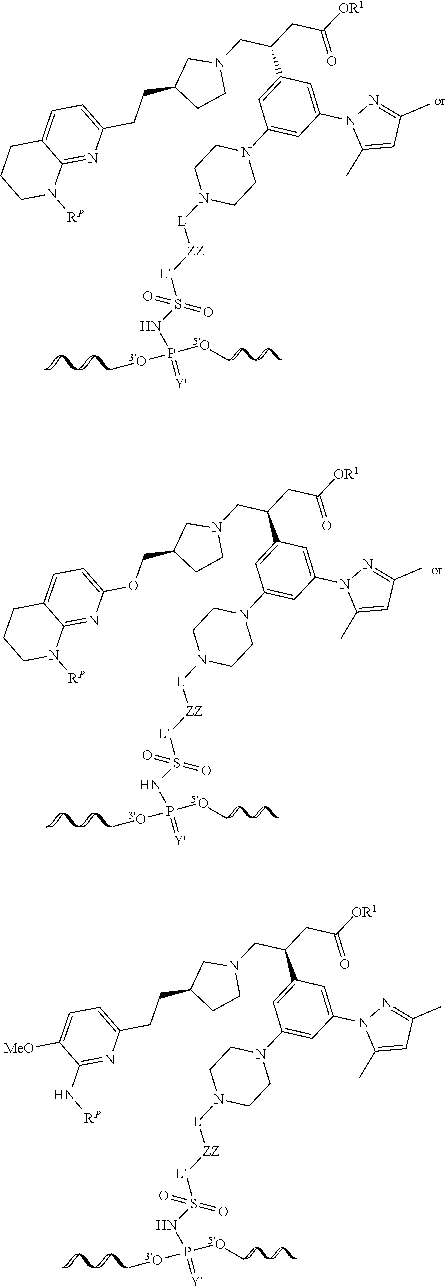

wherein Y′ is O or S, and R¹, L, L′, and ZZ are as defined for Formula (X). In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound is

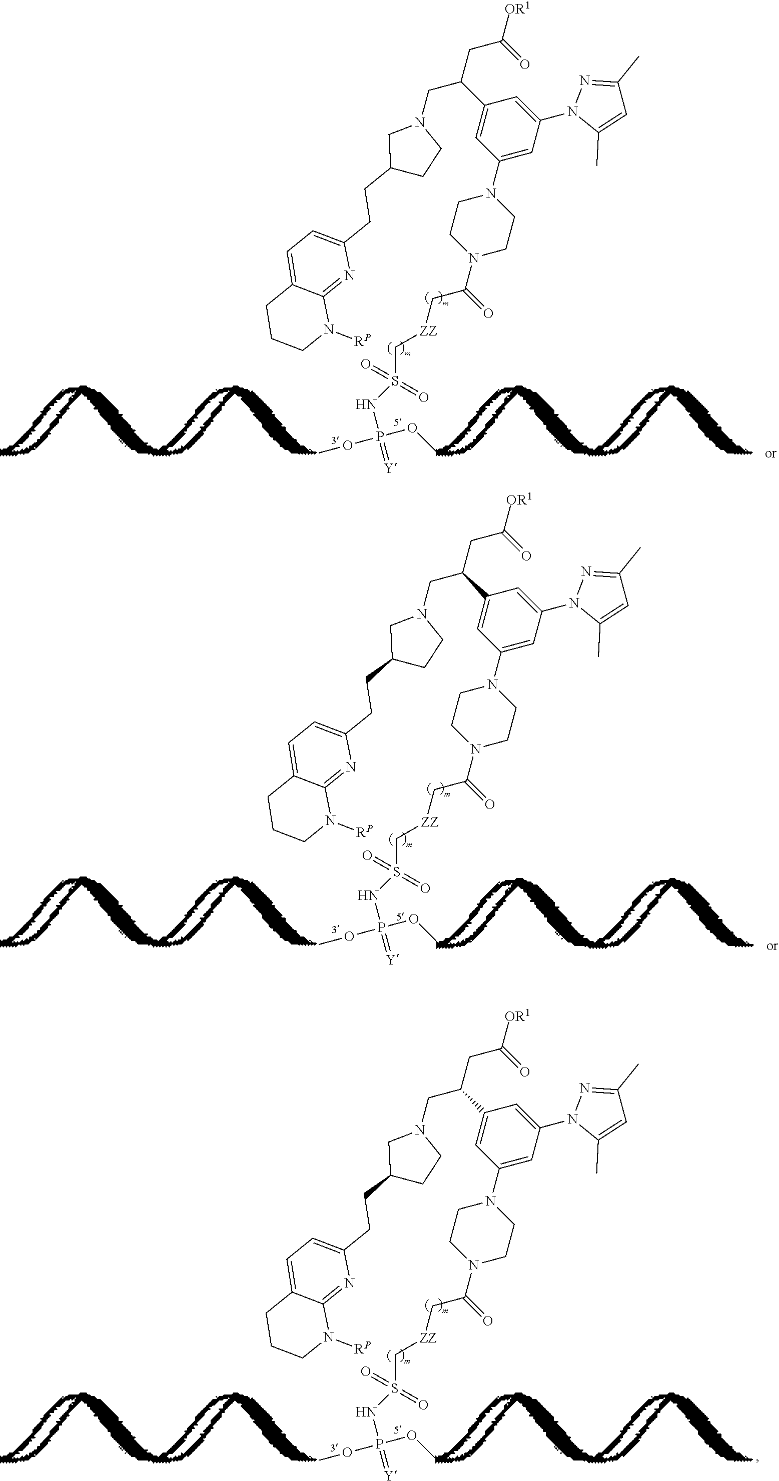

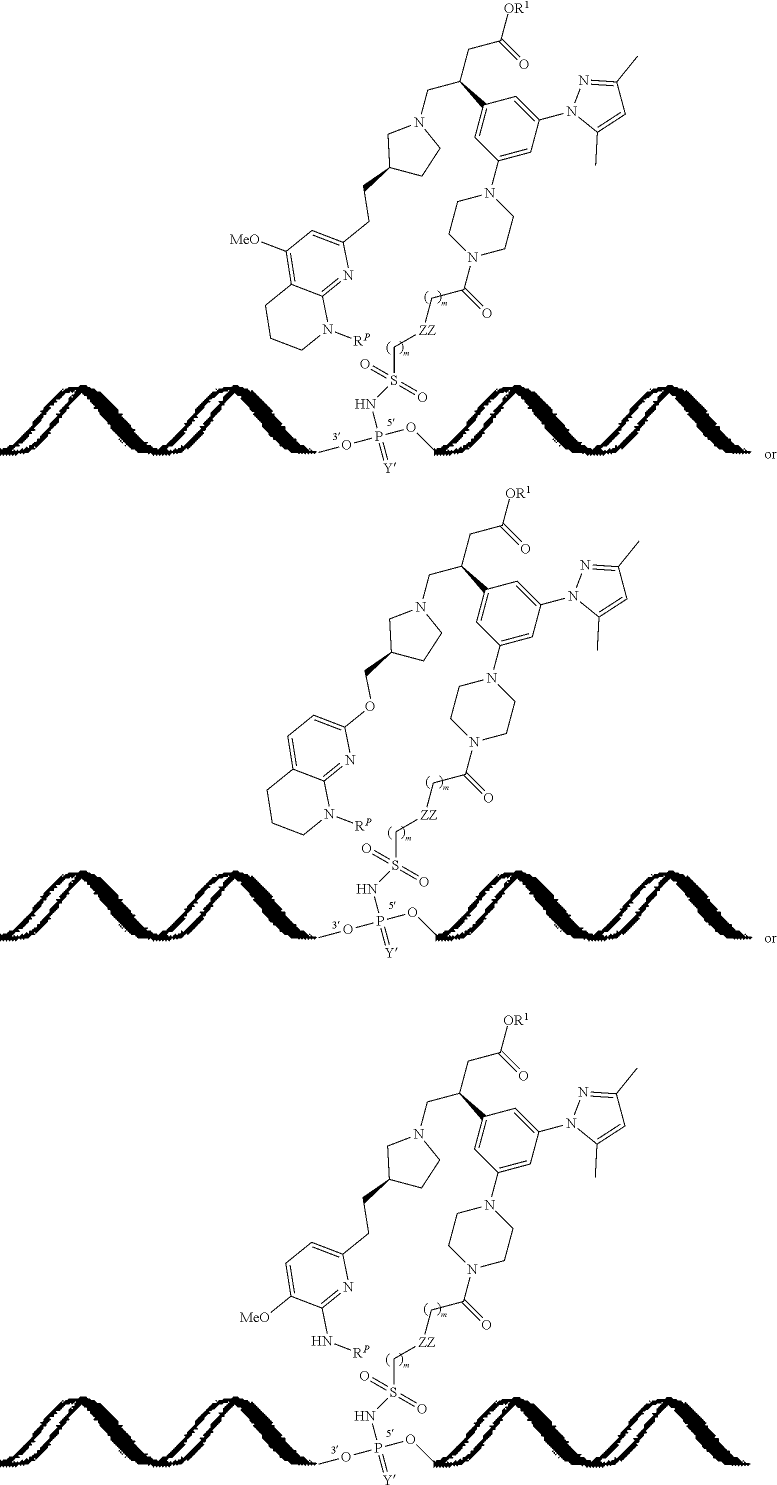

wherein each m is an integer selected from 1-20 (e.g., 2-20, 2-10, 1-10, 2-16, 4-16, 4-8, or 6-12), Y′ is O or S, and R¹, L, L′, and ZZ are as defined for Formula (X).

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

For Formula (X), when R^{T1} is -L^{L}-oligonucleotide and is conjugated at the 5′-end of the oligonucleotide, in one embodiment, R^{T1} can be represented as Formula (X-5′)

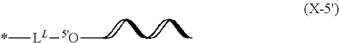

wherein L^{L} is —P(Y)(OH)—, wherein Y is O or S (e.g., S); and * represents the bond to remainder of the compound of Formula (X).

In another embodiment of Formula (X), when R^{T1} is -L^{L}-oligonucleotide and is conjugated at the 5′-end of the oligonucleotide, in one embodiment, R^{T1} can be represented as Formula (X-5′o) or Formula (X-5's),

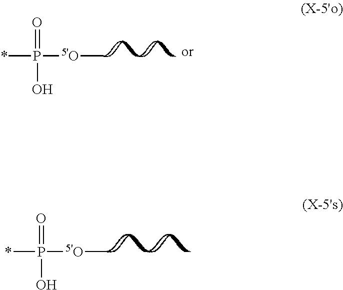

wherein * represents the bond to remainder of the compound of Formula (X).

In an embodiment of Formula (X-5′), (X-5′o), and Formula (X-5's), -L-ZZ-L′-R^{T} represents any one of Formulae (x-a) through (x-s). In representative embodiments, -L-ZZ-L′-R^{T} represents Formula (x-1),

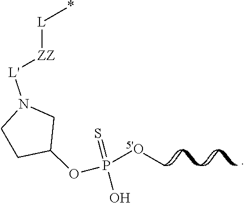

In other embodiments-L-ZZ-L′-R^{T} represents one of the following formulae:

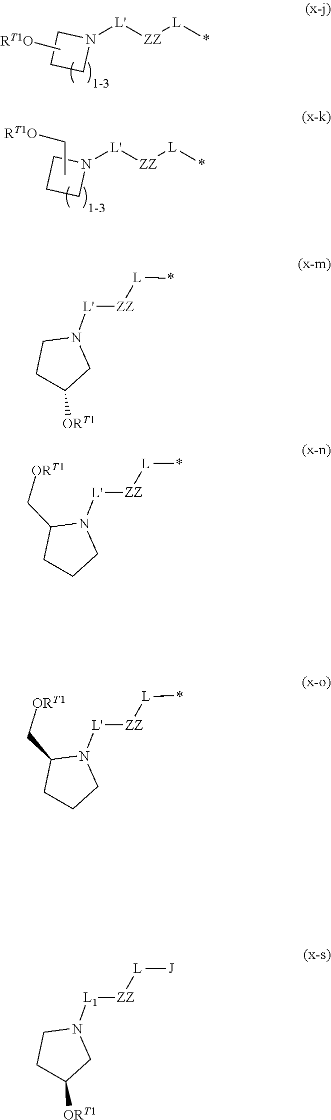

In other embodiments of Formula (X-5′), (X-5′o), and Formula (X-5's), L-ZZ-L′-R^{T} represents,

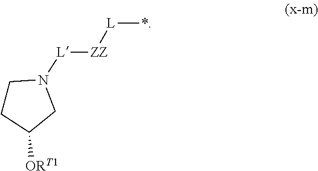

In other embodiments of Formula (X-5′), (X-5′o), and Formula (X-5's), L-ZZ-L′-R^{T} represents,

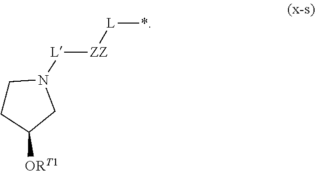

In another embodiment, the compound of Formula (X) is

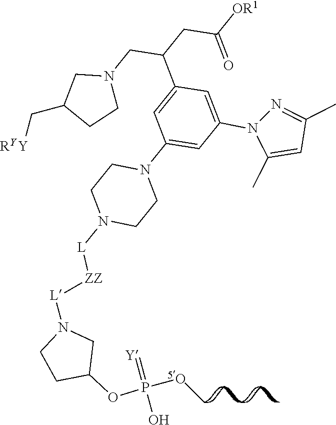

wherein Y′ is O or S, and R^{Y}, Y, R¹, L, L′, and ZZ are as defined for formula (X) or any embodiment thereof.

For Formula (X), when R^{T1} is -L^{L}-oligonucleotide and is conjugated at the 3′-end of the oligonucleotide, in one embodiment, R^{T1} can be represented as Formula (X-3′)

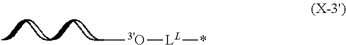

wherein L^{L} is —P(Y)(OH)—, wherein Y is O or S (e.g., S); and * represents the bond to remainder of the compound of Formula (X).

In another embodiment of Formula (X), when R^{T1} is -L^{L}-oligonucleotide and is conjugated at the 3′-end of the oligonucleotide, in one embodiment, R^{T1} can be represented as Formula (X-3′o) or Formula (X-3's),

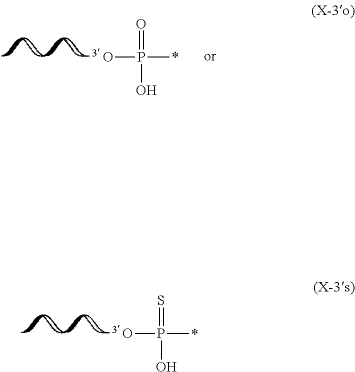 [SMILES: *P(=O)(O)O ; *P(O)(O)=S]

wherein * represents the bond to remainder of the compound of Formula (X).

In an embodiment of Formula (X-3′), (X-3′o), and Formula (X-3's), -L-ZZ-L′-R^{T} represents any one of Formulae (x-a) through (x-ab).

In representative embodiments, -L-ZZ-L′-R^{T} represents Formula (x-e),

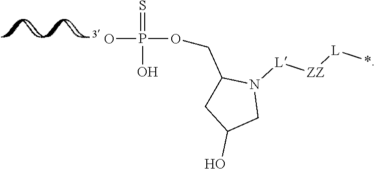

In other embodiments-L-ZZ-L′-R^{T} represents one of the following formulae:

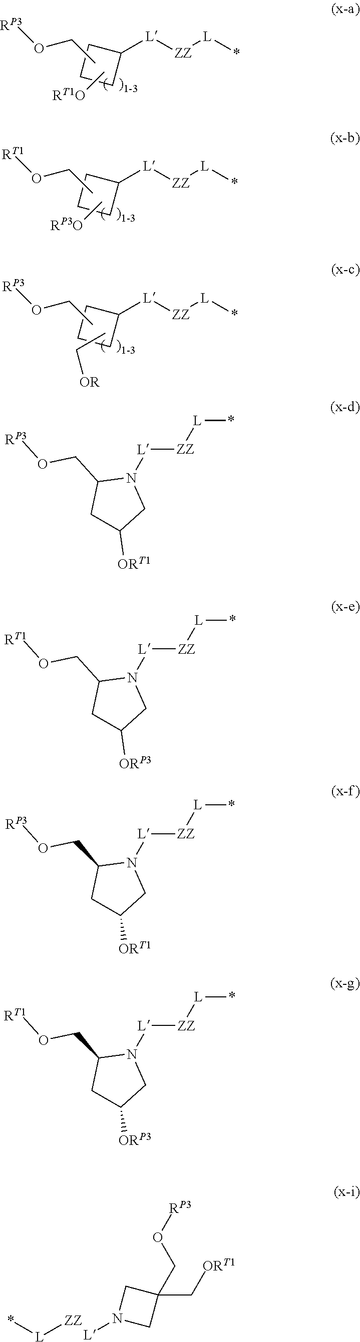

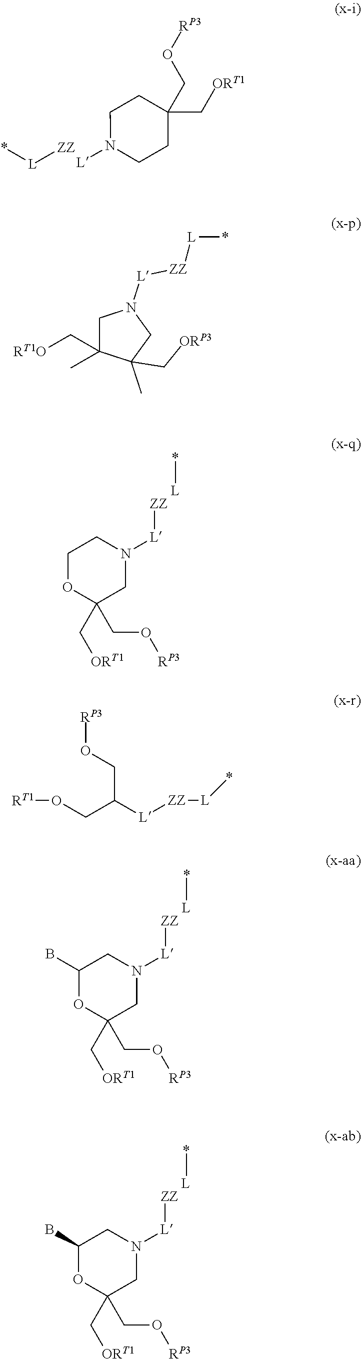

In other embodiments of Formula (X-3′), (X-3′o), and Formula (X-3's), L-ZZ-L′-R^{T} represents,

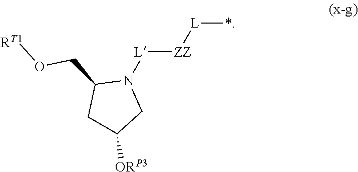

In other embodiments of Formula (X-3′), (X-3′o), and Formula (X-3's), L-ZZ-L′-R^{T} represents,

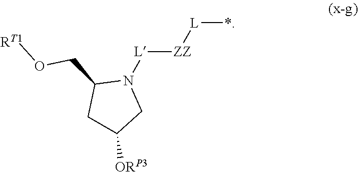

In other embodiments of Formula (X-3′), (X-3′o), and Formula (X-3's), L-ZZ-L′-R^{T} represents,

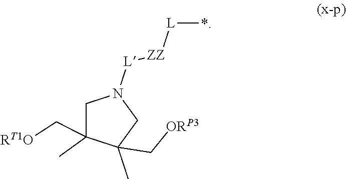

In other embodiments of Formula (X-3′), (X-3′o), and Formula (X-3's), L-ZZ-L′-R^{T} represents,

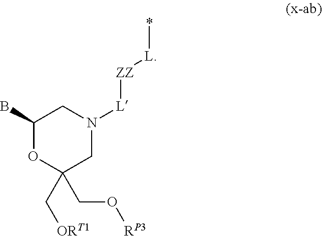

In other embodiments of Formula (X-3′), (X-3′o), and Formula (X-3's), L-ZZ-L′-R^{T} represents,

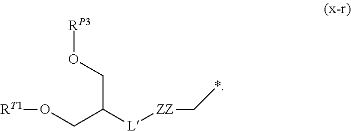

In another embodiment of Formula (X), R^{L} is

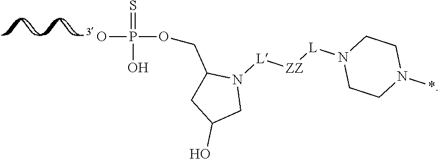

In another embodiment, the compound of Formula (X) is

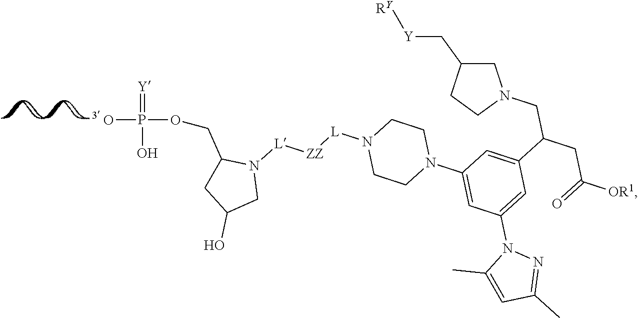

wherein Y′ is O or S, and R^{Y}, R¹, Y, L′, ZZ, and L are as defined for Formula (X) or any embodiment thereof. In one embodiment, R¹ is hydrogen. In another embodiment, R¹ is C₁₋₆alkyl (e.g., methyl or t-butyl).

In another embodiment, the compound of Formula (X) is

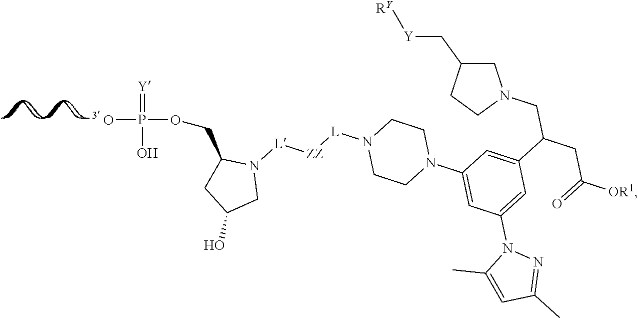

wherein Y′ is O or S, and R^{Y}, Y, R¹, L′, ZZ, and L are as defined for Formula (X) or any embodiment thereof

In another embodiment, the compound of Formula (X) is

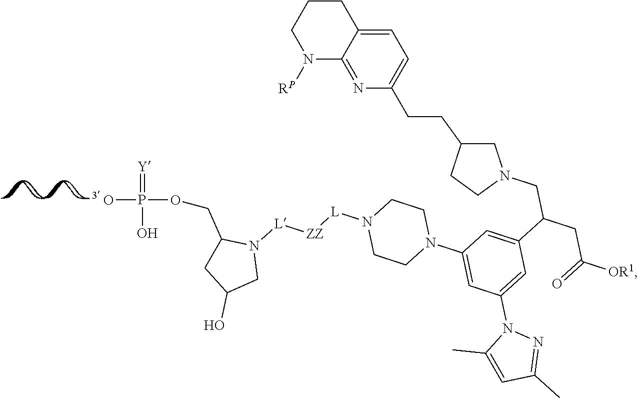

wherein Y′ is O or S, and R^{Y}, R¹, Y, L′, ZZ, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

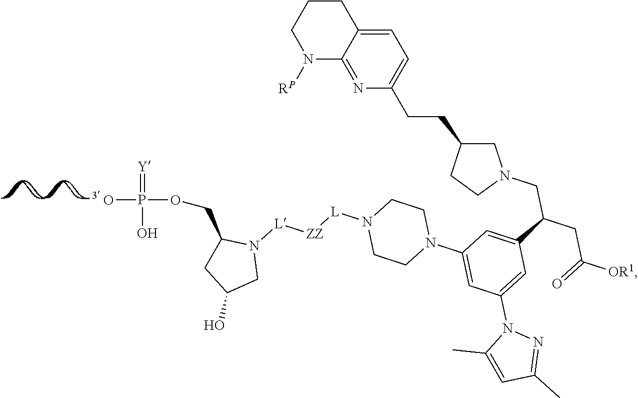

wherein Y′ is O or S, and R^{P}, R¹, L′, ZZ, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

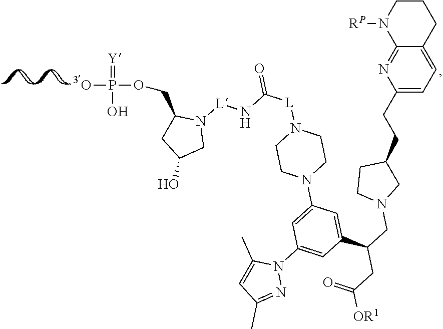

wherein Y′ is O or S, and R^{P}, R¹, L′, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

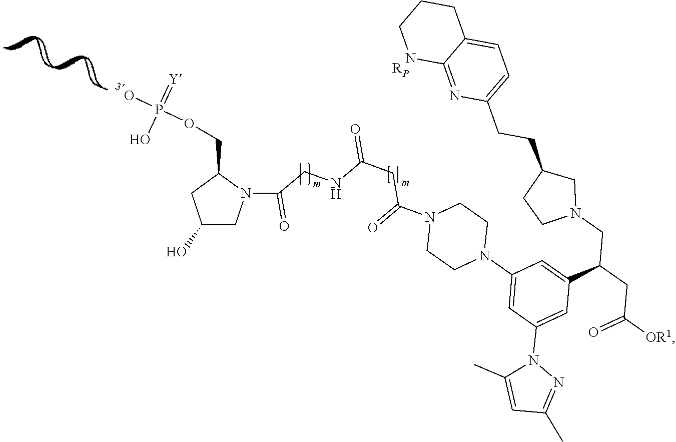

wherein each m is independently an integer selected from 1-10; Y′ is O or S, and R^{P} and R¹ are as defined for Formula (X) or any embodiment thereof. In one embodiment, each m is independently an integer selected from 2-10; or 2-8, or 2-6.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

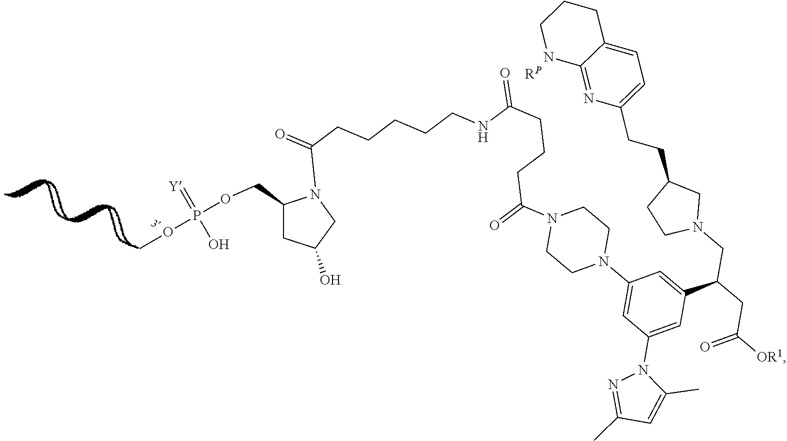

wherein Y′ is O or S, and R^{P} and R¹ are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

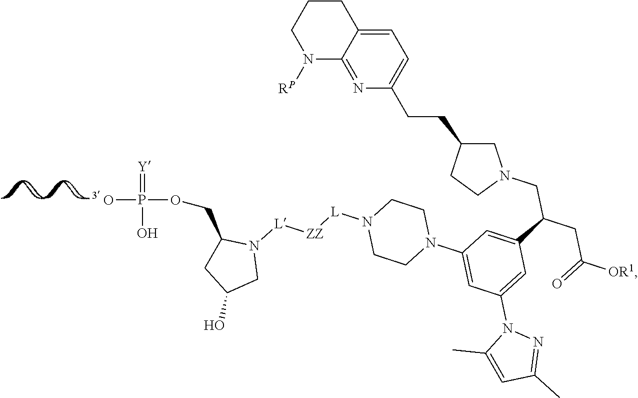

wherein Y′ is O or S, and R^{P}, R¹, L′, ZZ, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

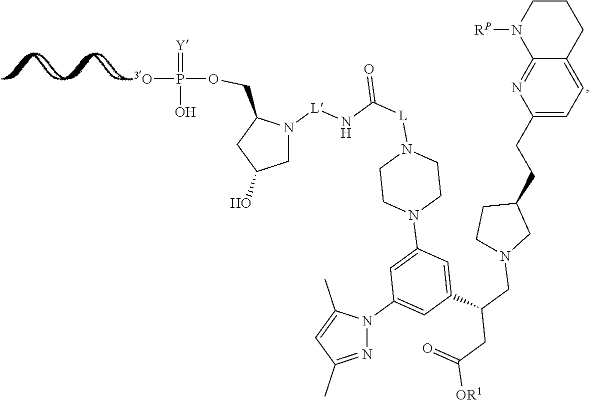

wherein Y′ is O or S, and R^{P}, R¹, L′, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

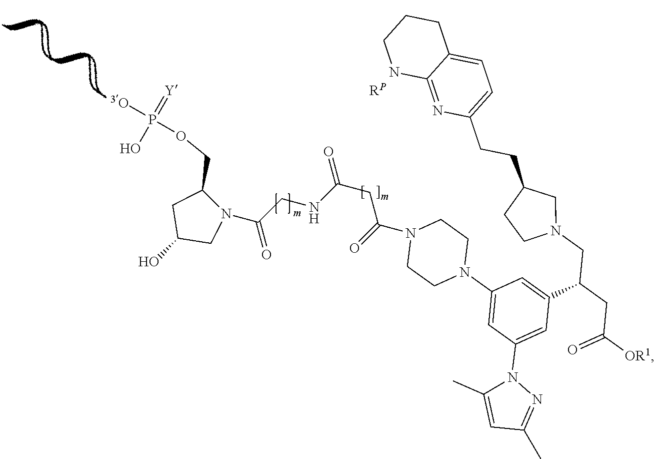

wherein each m is independently an integer selected from 1-10; Y′ is O or S, and R^{P} and R¹ are as defined for Formula (X) or any embodiment thereof. In one embodiment, each m is independently an integer selected from 2-10; or 2-8, or 2-6.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

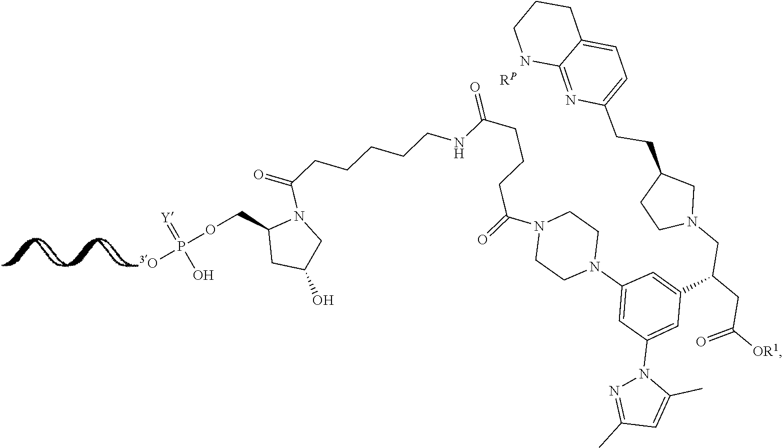

wherein Y′ is O or S, and R^{P} and R¹ are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

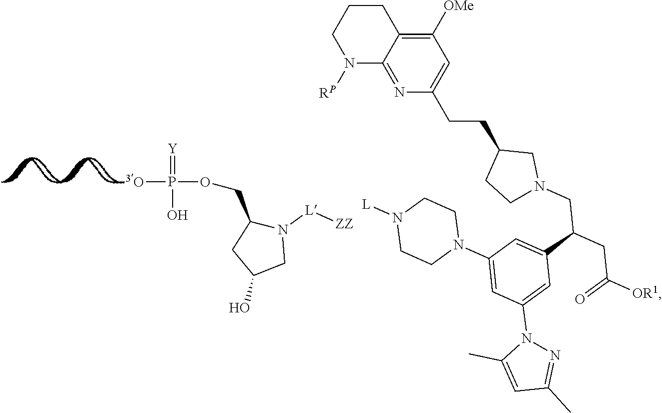

wherein Y′ is O or S, and R^{P}, R¹, L′, ZZ, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

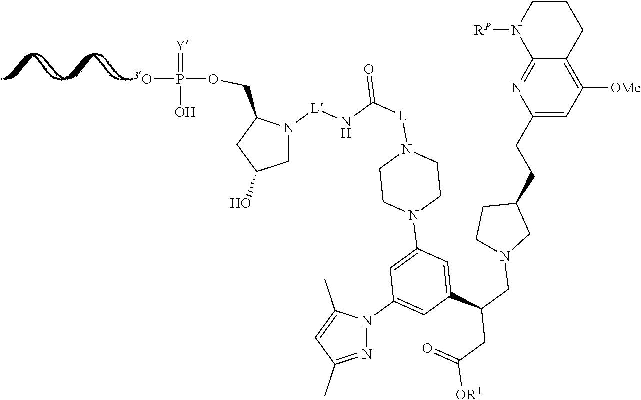

wherein Y′ is O or S, and R^{P}, R¹, L′, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

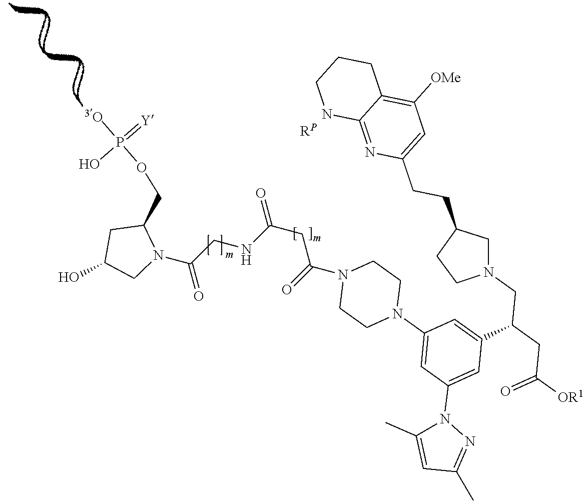

wherein each m is independently an integer selected from 1-10; Y′ is O or S, and R^{P} and R¹, are as defined for Formula (X) or any embodiment thereof. In one embodiment, each m is independently an integer selected from 2-10; or 2-8, or 2-6.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

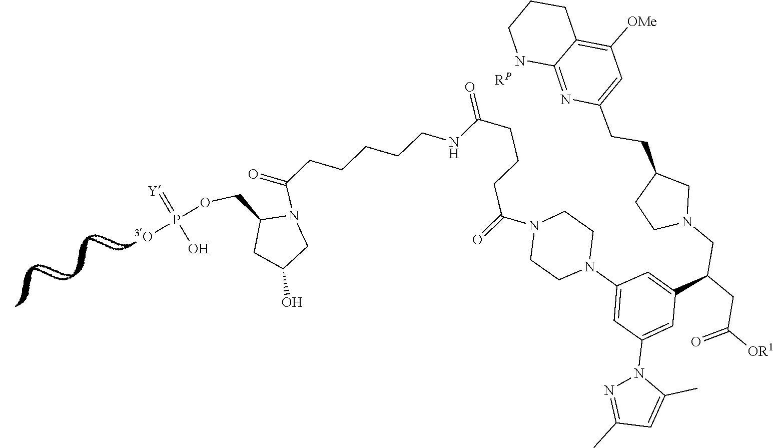

wherein Y′ is O or S, and R^{P} and R¹ are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

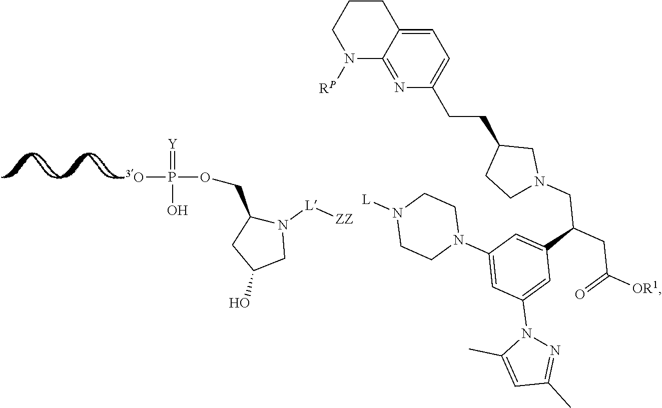

wherein Y′ is O or S, and R^{P}, R¹, L′, ZZ, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

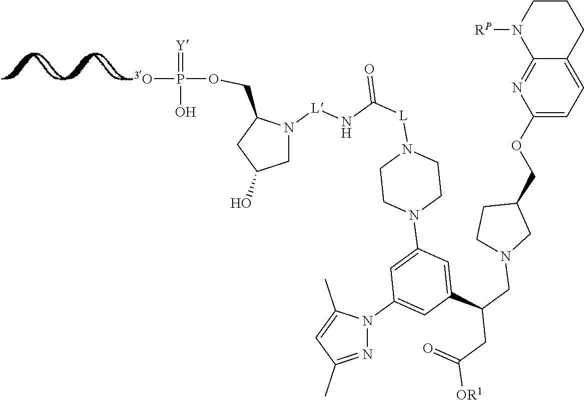

wherein Y′ is O or S, and R^{P}, R¹, L′, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

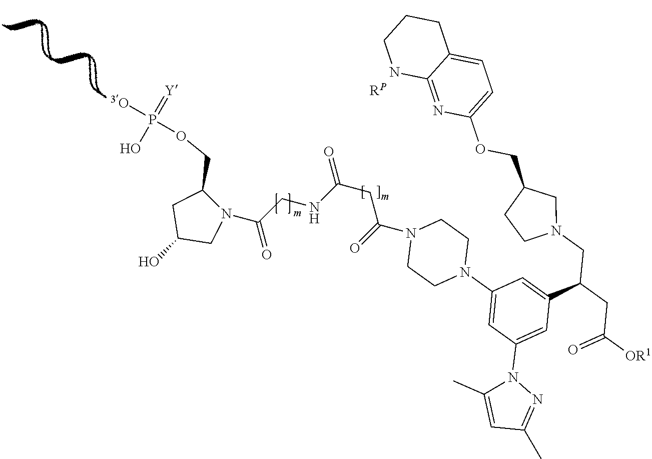

wherein each m is independently an integer selected from 1-10; Y′ is O or S, and R^{P} and R¹, are as defined for Formula (X) or any embodiment thereof. In one embodiment, each m is independently an integer selected from 2-10; or 2-8, or 2-6.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

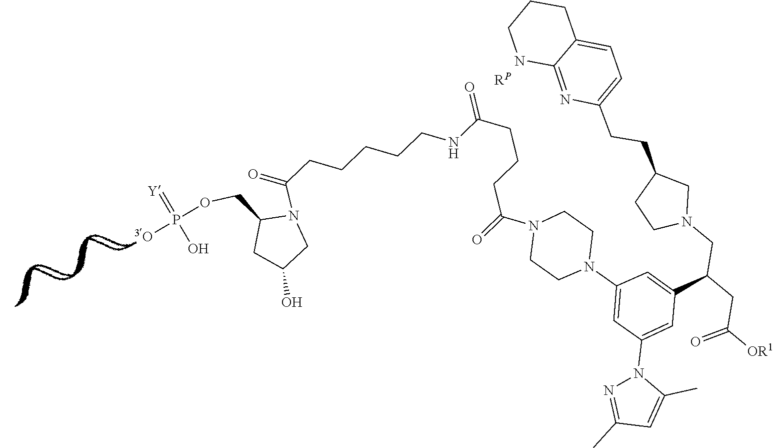

wherein Y′ is O or S, and R^{P} and R¹ are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

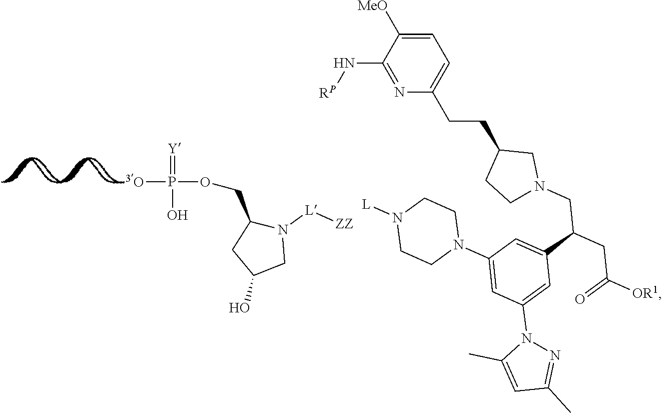

wherein Y′ is O or S, and R^{P}, R¹, L′, ZZ, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

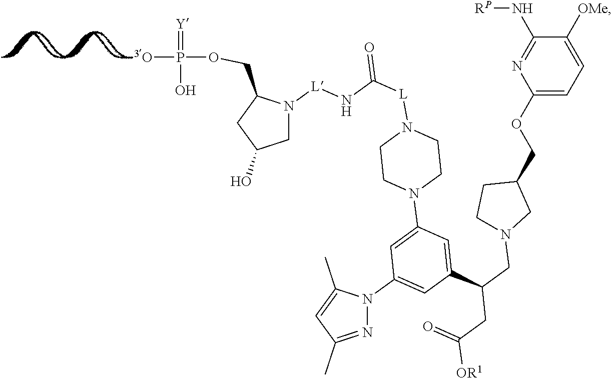

wherein Y′ is O or S, and R^{P}, R¹, L′, and L are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

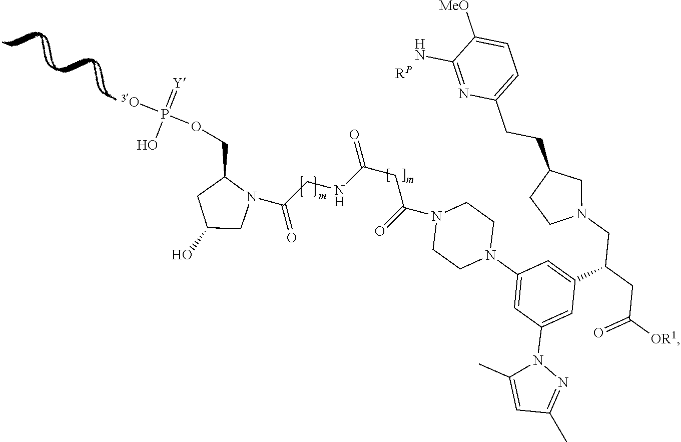

wherein each m is independently an integer selected from 1-10; Y′ is O or S, and R^{P} and R¹ are as defined for Formula (X) or any embodiment thereof. In one embodiment, each m is independently an integer selected from 2-10; or 2-8, or 2-6.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

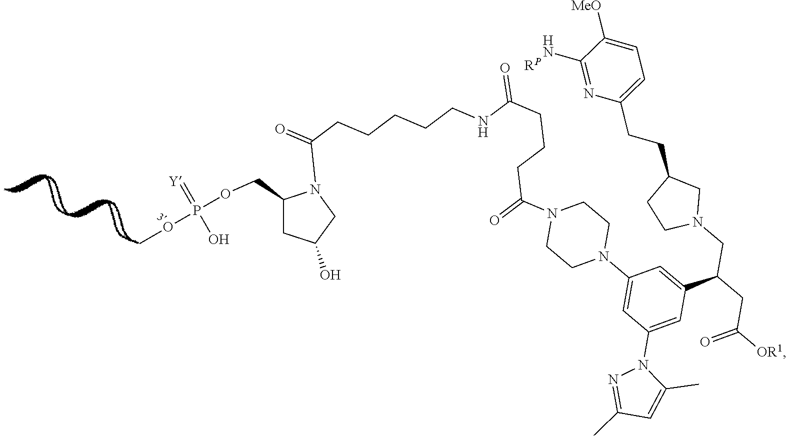

wherein Y′ is O or S, and R^{P} and R¹ are as defined for Formula (X) or any embodiment thereof.

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

### R^{T1}, Phosphorous Coupling Groups

In another embodiment, R^{P3}, when present, is a hydroxyl protecting group (e.g., according to any one of the preceding embodiments of R^{P3}) and R^{T1} is phosphorous coupling group

In another embodiment, R^{P3}, when present, is a hydroxyl protecting group (e.g., according to any one of the preceding embodiments of R^{P3}) and R^{T1} is phosphorous coupling group of the formula —P(Z)(X), wherein:
X is selected from the group consisting of
C₁₋₆alkyl (e.g., methyl),
C₁₋₆alkoxyC₁₋₆alkyl (e.g., 3-methoxypropyl),
C₁₋₆alkoxy (e.g., —OCH₃, —OCH₂CH₃, —OCH₂CH₂CH₃, —OCH₂CH(CH₃)₂), C₁₋₆alkoxy substituted with R (e.g., —OCH₂CH₂CN, —OCH₂CH₂Si(CH₃)₃, —OCH₂CH₂Si(CH₂CH₃)₃,

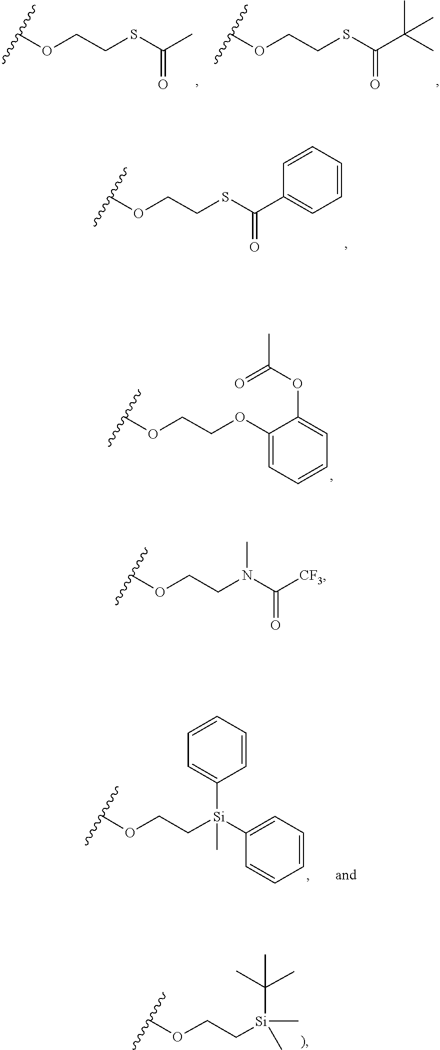

C₂₋₆alkenyloxy (e.g., —OC(H)═CH₂, —OCH₂C(H)═CH₂), phenoxy optionally substituted with 1, 2, 3, or 4 R groups.

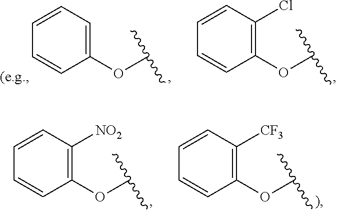 [SMILES: *Oc1ccccc1 ; *Oc1ccccc1Cl ; *Oc1ccccc1[N+](=O)[O-] ; *Oc1ccccc1C(F)(F)F]

benzyloxy optionally substituted with 1, 2, 3, or 4 R groups.

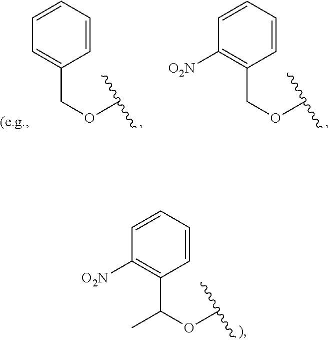 [SMILES: *OCc1ccccc1 ; *OCc1ccccc1[N+](=O)[O-] ; *OC(C)c1ccccc1[N+](=O)[O-]]

and
Z is selected from the group consisting of di(C₁₋₆alkyl)amino

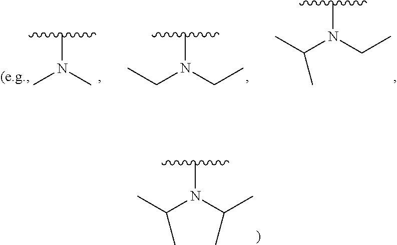

and heterocyclyl optionally substituted with 1, 2, 3, or 4 R groups.

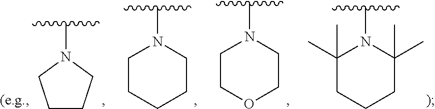

or
X and Z taken together with the phosphorus atom to which they are attached form a cyclic monocyclic or bicyclic heterocyclyl group that is optionally substituted with 1, 2, 3, or 4 R groups.

In another embodiment, R^{P3}, when present, is a hydroxyl protecting group (e.g., according to any one of the preceding embodiments of R^{P3}) and R^{T1} is phosphorous coupling group of the formula,

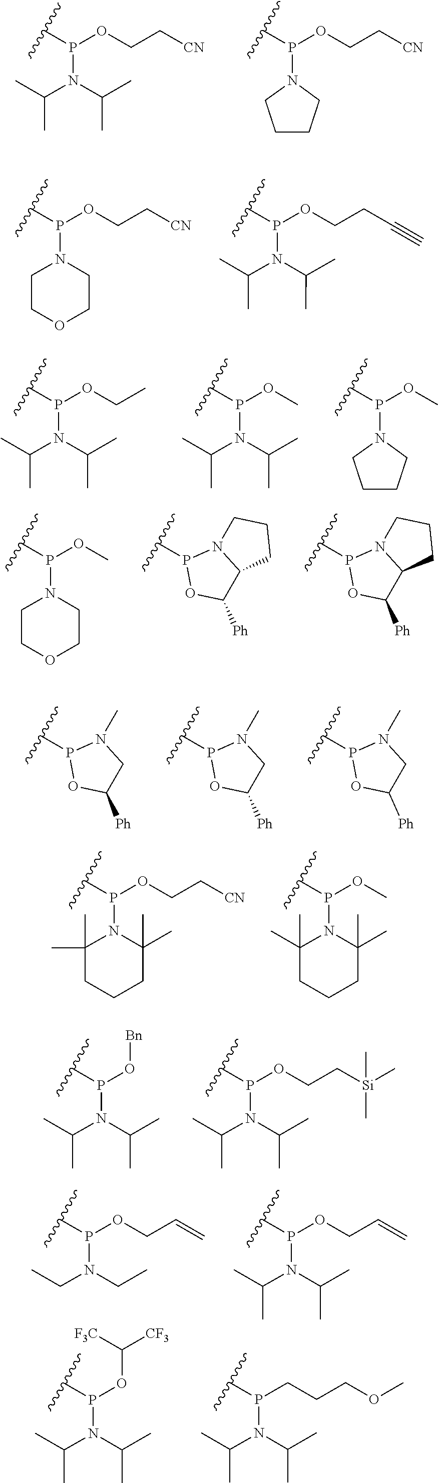

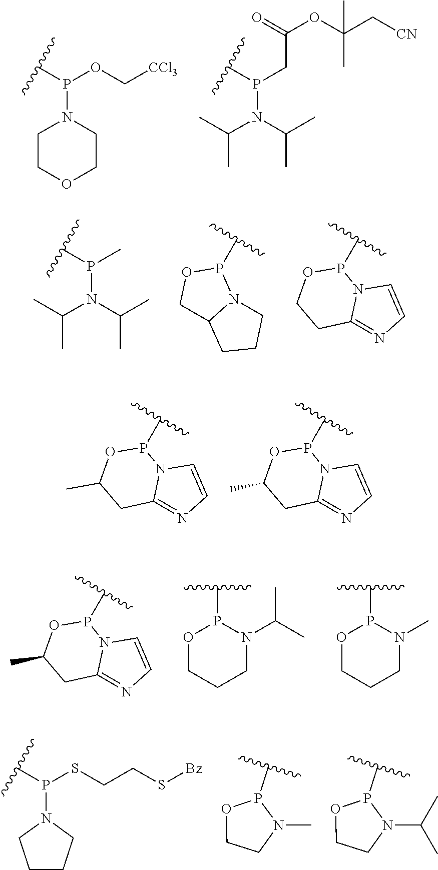

In another embodiment, R^{P3}, when present, is a hydroxyl protecting group (e.g., according to any one of the preceding embodiments of R^{P3}) and R^{T1} is

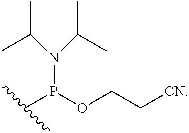 [SMILES: *P(OCCC#N)N(C(C)C)C(C)C]

In another embodiment, R^{P3}, when present, is a hydroxyl protecting group (e.g., according to any one of the preceding embodiments of R^{P3}) and R^{T1} is phosphorous coupling group of the formula

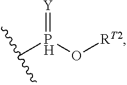

or a salt thereof, wherein Y is O or S; and R^{T2} is hydrogen or —C(O)C₁₋₆alkyl.

In another embodiment, R^{P3}, when present, is a hydroxyl protecting group (e.g., according to any one of the preceding embodiments of R^{P3}) and R^{T1} is phosphorous coupling group of the formula

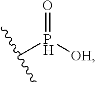

or a salt thereof.

### Solid Supports

In another embodiment, R^{P3}, when present, is a hydroxyl protecting group (e.g., according to any one of the preceding embodiments of R^{P3}) and R^{T1} is -L^{K}-S^{S}, wherein L^{K} is a support linking group and S^{S} is a solid support, —OR^{SS} or —N(R^{SS})₂, or hydrogen, wherein each R^{SS} is independently hydrogen or C₁₋₆alkyl.

In another embodiment, R^{P3}, when present, is a hydroxyl protecting group (e.g., according to any one of the preceding embodiments of R^{P3}) and R^{T1} is -L^{K}-S^{S}, wherein L^{K} is a support linking group and SS is —OR^{SS} or —N(R^{SS})₂.

In another embodiment, R^{P3}, when present, is a hydroxyl protecting group (e.g., according to any one of the preceding embodiments of R^{P3}) and R^{T1} is -L^{K}-S^{S}, wherein L^{K} is a support linking group of the formula: —C(O)(CH₂)ₙC(O)—, wherein n is 1-20; and S^{S} is —OR^{SS} (e.g., —OH).

In another embodiment, R^{P3}, when present, is a hydroxyl protecting group (e.g., according to any one of the preceding embodiments of R^{P3}) and R^{T1} is -L^{K}-S^{S}, wherein L^{K} is a support linking group of the formula: —C(O)CH₂CH₂C(O)—, wherein n is 1-20; and S^{S} is —OR^{SS} (e.g., —OH).

In another embodiment, R^{P3}, when present, is a hydroxyl protecting group (e.g., according to any one of the preceding embodiments of R^{P3}) and R^{T1} is -L^{K}-S^{S}, wherein S^{S} is a solid support.

In another embodiment, R^{P3}, when present, is a hydroxyl protecting group (e.g., according to any one of the preceding embodiments of R^{P3}) and R^{T1} is -L^{K}-S^{S}, wherein S^{S} is a controlled pore glass (CPG),

In another embodiment, R^{P3}, when present, is a hydroxyl protecting group (e.g., according to any one of the preceding embodiments of R^{P3}) and R^{T1} is -L^{K}-S^{S}, wherein S^{S} is a polystyrene (e.g., cross-linked polystyrene).

In an embodiment of each of the preceding, L^{K} is

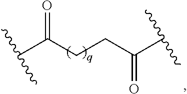

wherein q is 0 or an integer selected from 1-20. In an embodiment of each of the preceding, L^{K} is

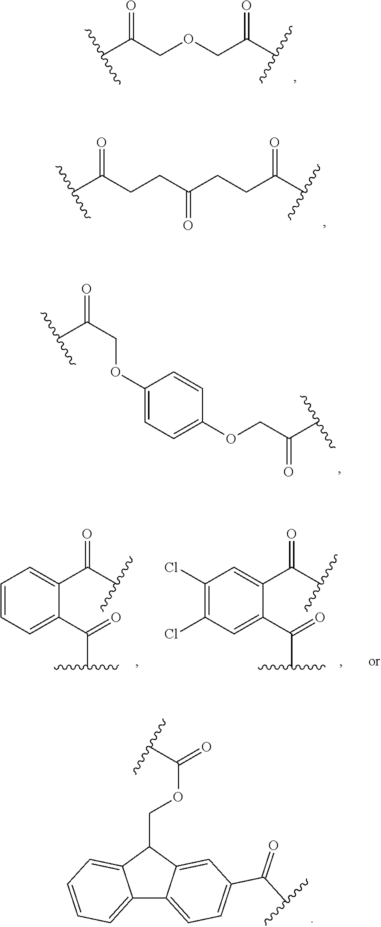 [SMILES: *C(=O)COCC(*)=O ; *C(=O)CCC(=O)CCC(*)=O ; *C(=O)COc1ccc(OCC(*)=O)cc1 ; *C(=O)c1ccccc1C(*)=O ; *C(=O)c1cc(Cl)c(Cl)cc1C(*)=O ; *C(=O)OCC1c2ccccc2-c2ccc(C(*)=O)cc21]

In an embodiment of each of the preceding, L^{K} is

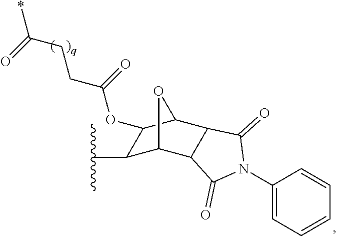

wherein q is 0 or an integer selected from 1-20, and * represents the bond to SS (i.e. to a functional group on the surface of S^{S}).

In an embodiment of each of the preceding, R^{P3}, when present, is a hydroxyl protecting group (e.g., according to any one of the preceding embodiments of R^{P3}) and R^{T} is

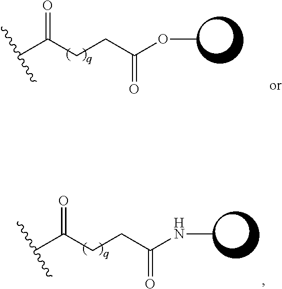

wherein [symbol] is the solid support.

In an embodiment of each of the preceding, R^{P3}, when present, is a hydroxyl protecting group (e.g., according to any one of the preceding embodiments of R^{P3}) and R^{T} is

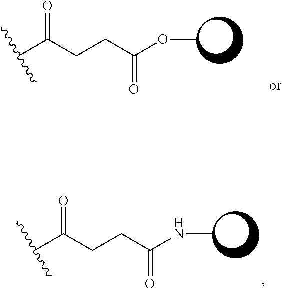

wherein [symbol] is the solid support.

In an embodiment of each of the preceding, R^{P3}, when present, is a hydroxyl protecting group (e.g., according to any one of the preceding embodiments of R^{P3}) and R^{T} is

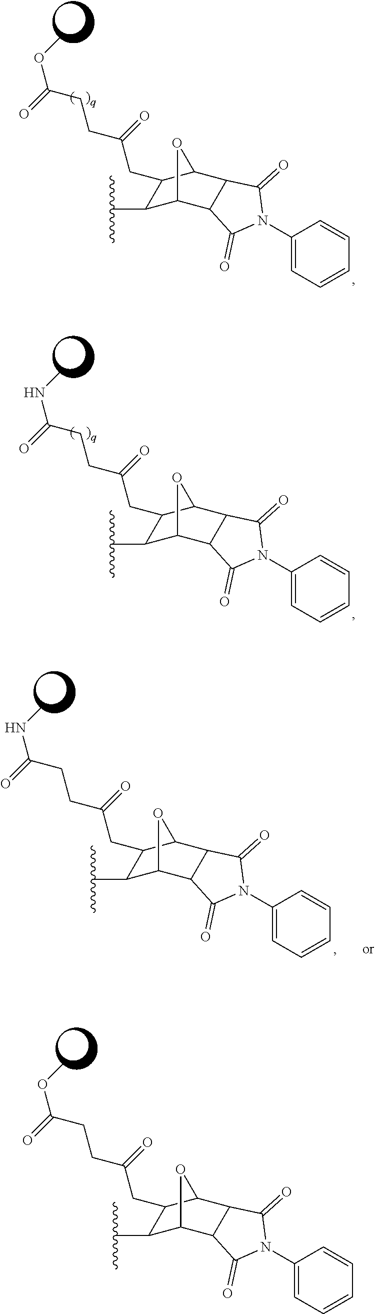

In another embodiment, the compound of Formula (X) is

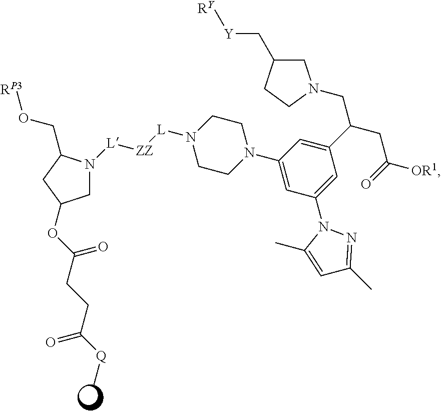

wherein [symbol] represents a solid support; Q is O or NH, and R¹, R^{Y}, Y, R^{P3}. L′, ZZ, and L are as defined for Formula (X) or any embodiment thereof. In one embodiment, R¹ is hydrogen. In another embodiment, R¹ is C₁₋₆alkyl (e.g., methyl or t-butyl). In one embodiment, R^{P3} is hydrogen. In another embodiment, R^{P3} is hydroxyl protecting group (e.g., 4,4′-dimethyoxytrityl (DMTr)).

In another embodiment, the compound of Formula (X) is

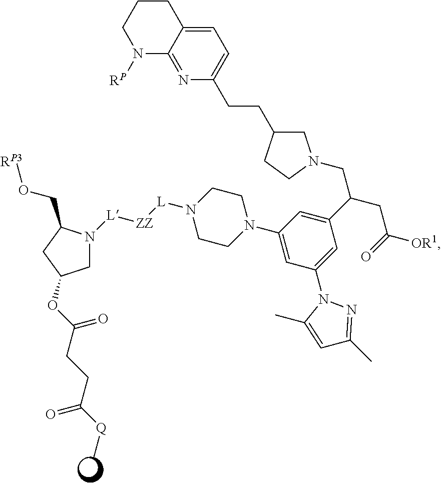

wherein [symbol] represents a solid support; Q is O or NH, and R¹, R^{P}, R^{P3}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R³ is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R³ is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

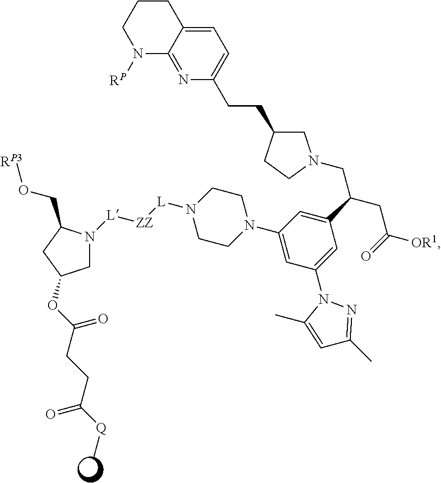

wherein [symbol] represents a solid support; Q is O or NH, and R¹, R^{P}, R^{P3}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

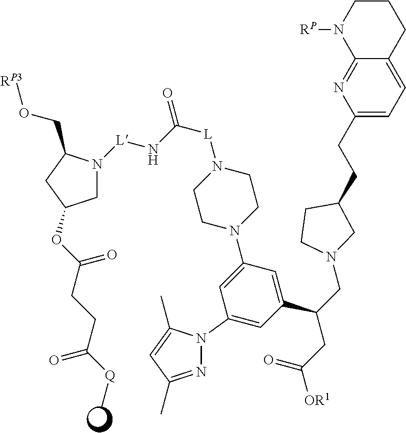

wherein [symbol] represents a solid support; Q is O or NH, and R^{P}, R^{P3}, R¹, L′, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen.

In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

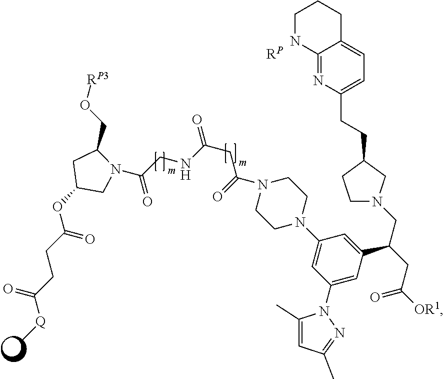

wherein each m is independently an integer selected from 1-10; [symbol] represents a solid support; Q is O or NH, and R^{P}, R^{P3}, and R¹ are as defined for Formula (X) or any embodiment thereof. In one embodiment, each m is independently an integer selected from 2-10; or 2-8, or 2-6.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen.

In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

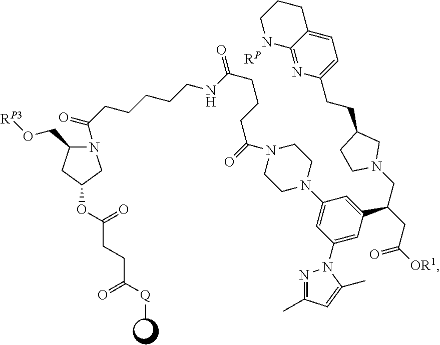

wherein [symbol] represents a solid support; Q is O or NH, and R^{P}, R^{P3} and R¹, are as defined for Formula (X) or any embodiment thereof and R^{P}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

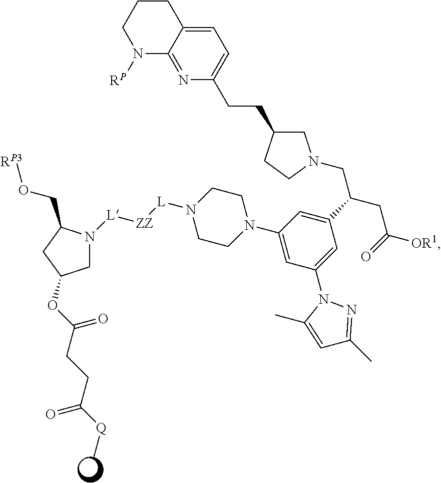

wherein [symbol] represents a solid support; Q is O or NH, and R^{P}, R^{P3}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁._alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁._alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

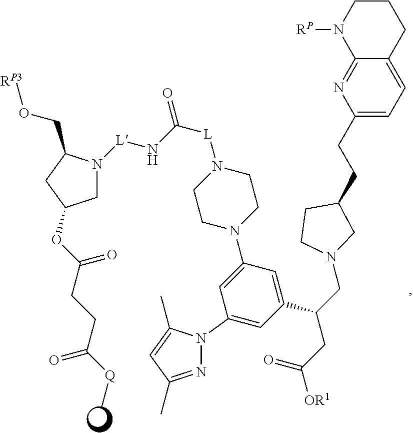

wherein [symbol] represents a solid support; Q is O or NH, and R^{P}, R^{P3}, R¹, L′ and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

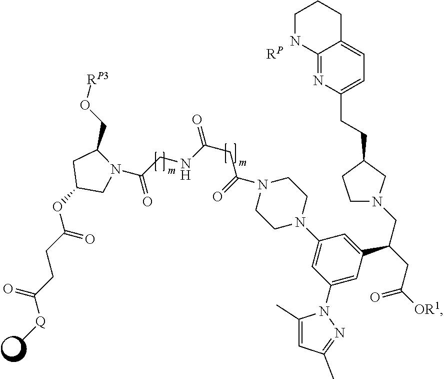

wherein each m is independently an integer selected from 1-10; [symbol] represents a solid support; Q is O or NH, and R^{P}, R^{P3}, and R¹ are as defined for Formula (X) or any embodiment thereof. In one embodiment, each m is independently an integer selected from 2-10; or 2-8, or 2-6.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

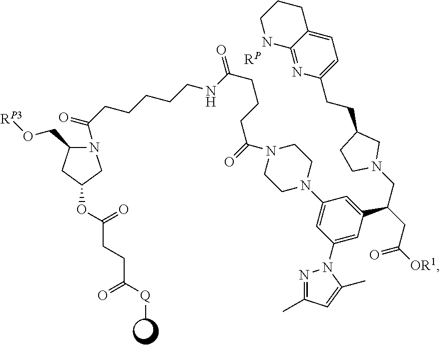

wherein [symbol] represents a solid support; Q is O or NH, and R^{P}, R^{P3}, and R¹ are as defined for Formula (X) or any embodiment thereof and R^{P}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

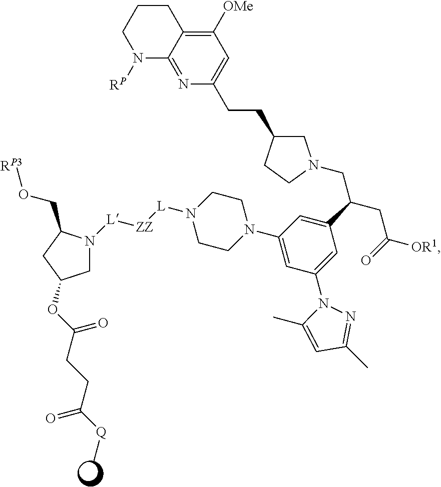

wherein [symbol] represents a solid support; Q is O or NH, and R^{P}, R^{P3}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

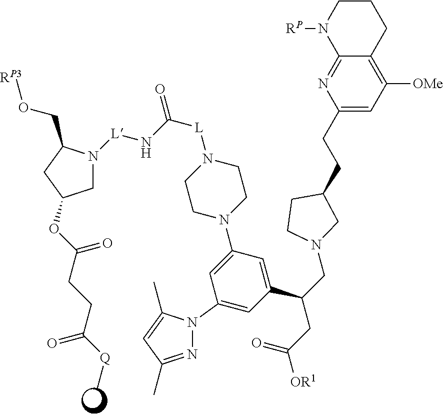

wherein [symbol] represents a solid support; Q is O or NH, and R^{P}, R^{P3}, R¹, L′, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

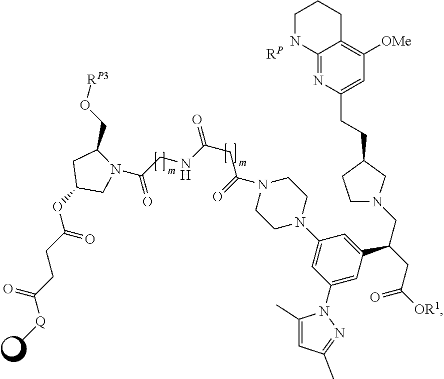

wherein each m is independently an integer selected from 1-10; [symbol] represents a solid support; Q is O or NH, and R^{P}, R^{P3}, and R¹ are as defined for Formula (X) or any embodiment thereof. In one embodiment, each m is independently an integer selected from 2-10; or 2-8, or 2-6.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

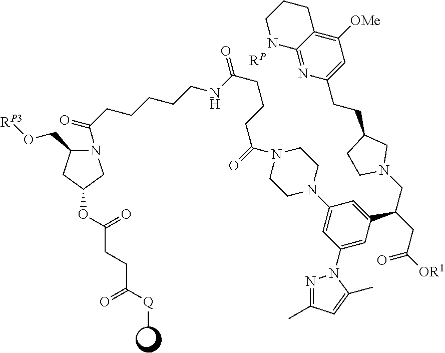

wherein [symbol] represents a solid support; Q is O or NH, and R^{P}, R^{P3}, and R¹ are as defined for Formula (X) or any embodiment thereof and R^{P}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen.

In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

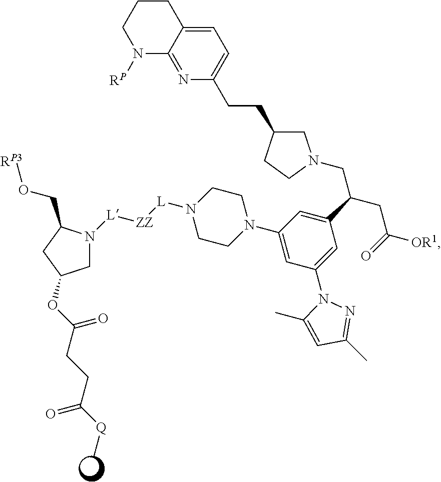

wherein [symbol] represents a solid support; Q is O or NH, and R^{P}, R^{P3}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen.

In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

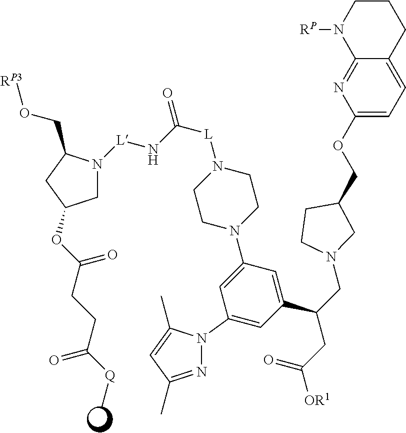

wherein [symbol] represents a solid support; Q is O or NH, and R^{P}, R^{P3}, R¹, L′, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

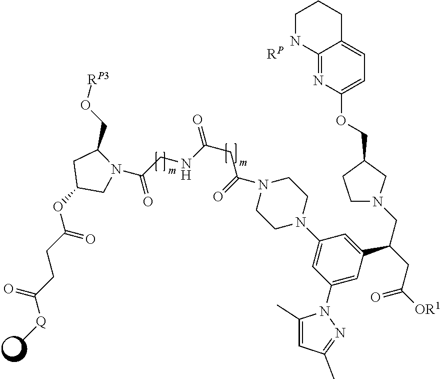

wherein each m is independently an integer selected from 1-10; [symbol] represents a solid support; Q is O or NH, and R^{P}, R^{P3}, and R¹ are as defined for Formula (X) or any embodiment thereof. In one embodiment, each m is independently an integer selected from 2-10; or 2-8, or 2-6.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

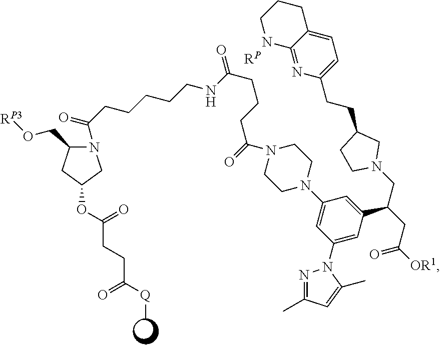

wherein [symbol] represents a solid support; Q is O or NH, and R^{P}, R^{P3}, and Rare as defined for Formula (X) or any embodiment thereof and R^{P}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen.

In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

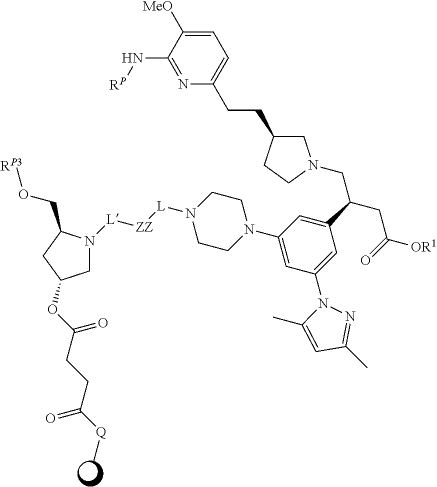

wherein [symbol] represents a solid support; Q is O or NH, and R¹, R^{P}, R^{P3}, R¹, L′, ZZ, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

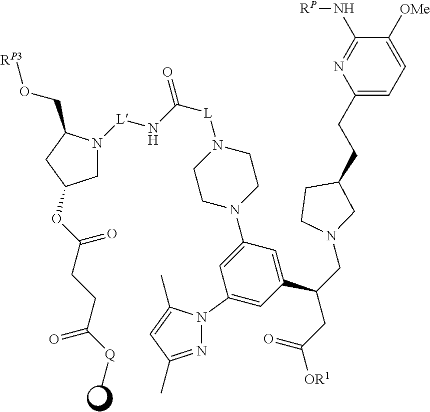

wherein [symbol] represents a solid support; Q is O or NH, and R^{P}, R^{P3}, R¹, L′, and L are as defined for Formula (X) or any embodiment thereof.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen. In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

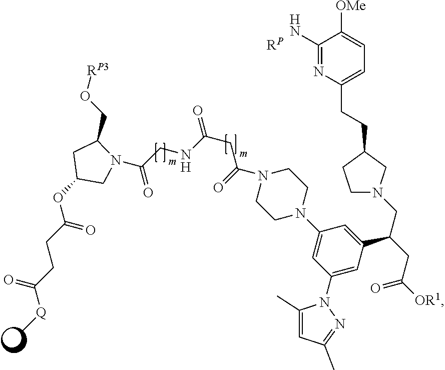

wherein each m is independently an integer selected from 1-10; [symbol] represents a solid support; Q is O or NH, and R^{P}, R^{P3}, and R¹ are as defined for Formula (X) or any embodiment thereof. In one embodiment, each m is independently an integer selected from 2-10; or 2-8, or 2-6.

In one embodiment, R^{P3} is a hydrogen. In another embodiment, R^{P3} is a hydroxyl protecting group (e.g., DMTr).

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, R^{P3} is hydrogen and R¹ is hydrogen. In one embodiment, R^{P3} is hydrogen and R¹ is C₁₋₆alkyl (e.g., methyl or t-butyl). In one embodiment, R^{P3} is a hydroxyl protecting group (e.g., DMTr) and R¹ is hydrogen. In one embodiment, R^{P3} is a hydroxyl protecting group (e.g., DMTr) and R¹ is C₁₋₆alkyl (e.g., methyl ort-butyl).

In one embodiment, R^{P3} is hydrogen and R^{P} is hydrogen. In one embodiment, R^{P3} is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr) and R^{P} is hydrogen. In one embodiment, R^{P3} is a hydroxyl protecting group (e.g., DMTr) and R^{P} is a nitrogen protecting group (e.g., t-butoxycarbonyl (Boc), benzyloxycarbonyl (Cbz) or phenoxyacetyl (pac)).

In one embodiment, R^{P3} is hydrogen, R¹ is hydrogen and R^{P} is hydrogen. In one embodiment, R^{P3} is a hydroxyl protecting group (e.g., DMTr), R¹ is hydrogen and R^{P} is hydrogen. In one embodiment, R^{P3} is hydrogen, R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is hydrogen and R^{P} is a nitrogen protecting group (e.g., t-butoxycarbonyl (Boc), benzyloxycarbonyl (Cbz) or phenoxyacetyl (pac)). In one embodiment, R^{P3} is hydrogen, R¹ is C₁₋₆alkyl (e.g., methyl or t-butyl) and R^{P} is hydrogen. In one embodiment, R^{P3} is a hydroxyl protecting group (e.g., DMTr), R¹ is C₁₋₆alkyl (e.g., methyl or t-butyl) and R^{P} is hydrogen.

In one embodiment, R^{P3} is hydrogen, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, R^{P3} is a hydroxyl protecting group (e.g., DMTr), R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (X) is

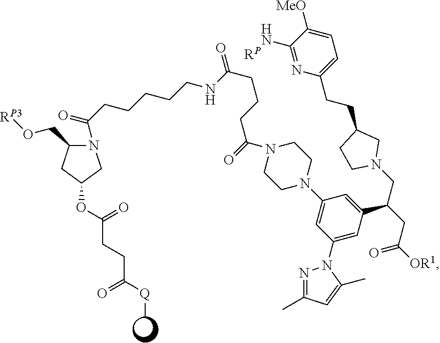

### ZZ Embodiments, Formula (X)

In embodiments of Formula (X), Formula (X-a) through (X-w), including embodiments of Formulae (x-a) through (x-s) and (xi-a) through (xi-m) ZZ is a linking group formed by a reactive pair. In some embodiments, ZZ comprises a group selected from the group consisting of

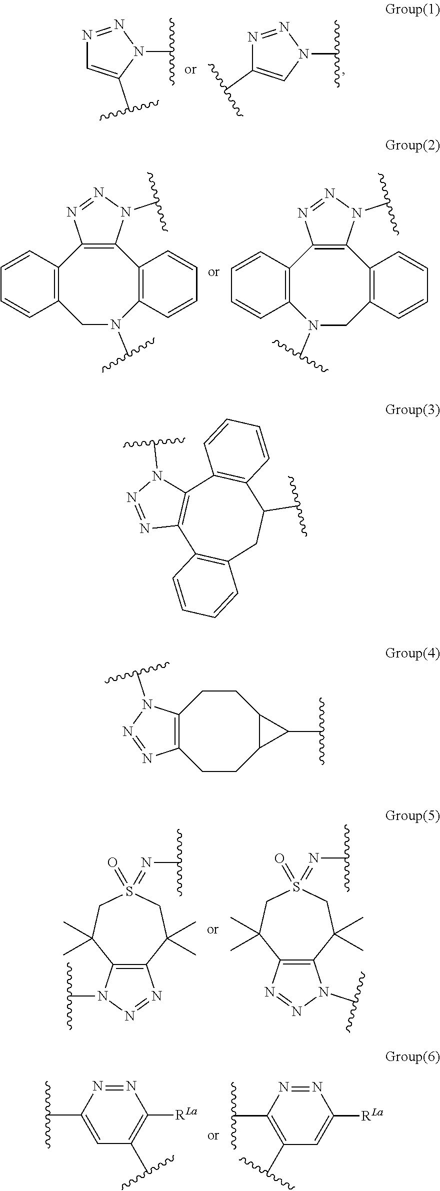

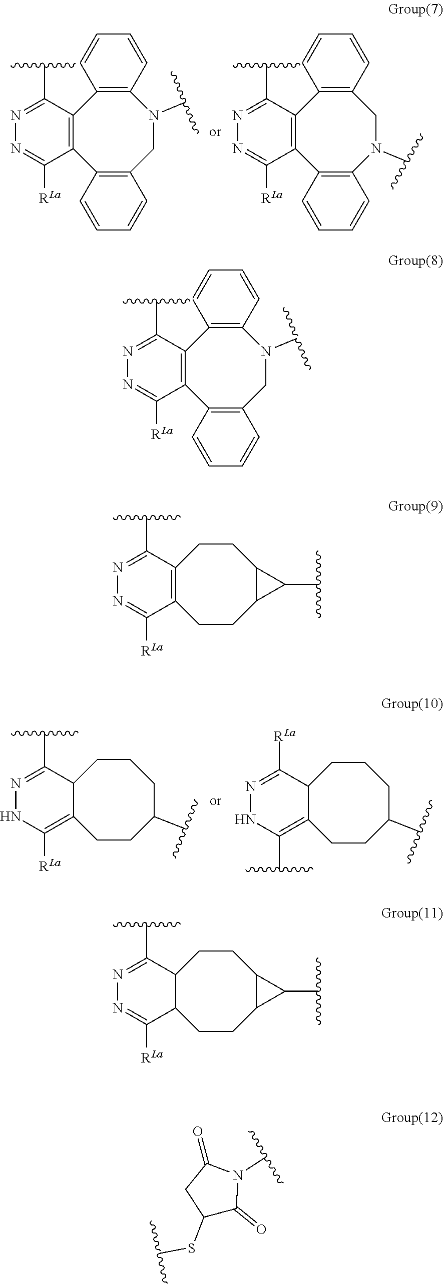

In some embodiments, ZZ comprises Group(1). In some embodiments, ZZ comprises Group(2). In some embodiments, ZZ comprises Group(3). In some embodiments, ZZ comprises Group(4). In some embodiments, ZZ comprises Group(5). In some embodiments, ZZ comprises Group(6). In some embodiments, ZZ comprises Group(7). In some embodiments, ZZ comprises Group(8). In some embodiments, ZZ comprises Group(9). In some embodiments, ZZ comprises Group(10). In some embodiments, ZZ comprises Group(11). In some embodiments, ZZ comprises Group(12).

In some embodiments, wherein ZZ is -A′-B′-A′-.

In some embodiments, ZZ is -A′-B′-A′-, wherein
each A′ is independently a bond, —O—, —S—, or —N(R^{N3})—, wherein R^{N3} is independently hydrogen or C₁₋₆alkyl and
each B′ is independently CH₂, C(O), C(S), S(O)₂, P(O)(OH), or P(S)(OH).

In some embodiments, ZZ is CH₂, C(O), C(S), S(O)₂, P(O)(OH), or P(S)(OH).

In some embodiments, ZZ is C(O), C(S), or S(O)₂.

In some embodiments, ZZ is P(O)(OH), or P(S)(OH).

In some embodiments, ZZ is —C(O)—.

In some embodiments, ZZ is -A′-B′- or -B′-A′- wherein
each A′ is independently —O—, —S—, or —N(R^{N3})—;
each B′ is independently CH₂, C(O), C(S), S(O)₂, P(O)(OH), or P(S)(OH); and
each R^{N3} is independently hydrogen or C₁₋₆alkyl.

In some embodiments, ZZ is -A′-B′- or -B′-A′- wherein
each A′ is independently —O— or —N(R^{N3})—, wherein R^{N3} is independently hydrogen or C₁₋₆ alkyl.
each B′ is independently CH₂, C(O), S(O)₂, P(O)(OH), or P(S)(OH); and
each R^{N3} is independently hydrogen or C₁₋₆alkyl.

In some embodiments, ZZ is —CH₂O—, —OCH₂—, —S—S—, —C═N—, —C═N—O—, —C═N—N(R^{N3}), —N═C—, —O—N═C—, —N(R^{N3})—N═C—, —C(O)N(R^{N3})—, —N(R^{N3})C(O)—, —C(O)O—, —OC(O)—, —OC(O)N(R^{N3}), —N(R^{N3})C(O)O—, —N(R^{N3})C(O)N(R^{N3})—, —S(O)₂N(R^{N3})—, —N(R^{N3})S(O)₂—, —OP(O)(OH)O—, —OP(S)(OH)O—, —OP(O)(OH)—, —OP(S)(OH)—, —P(O)(OH)O—, or —P(S)(OH)O—. wherein R^{N3} is independently hydrogen or C₁₋₆alkyl.

In some embodiments, ZZ is —CH₂O— or —OCH₂—.

In some embodiments, ZZ is —S—S—.

In some embodiments, ZZ is —C═N—, —C═N—O—, —C═N—N(R^{N3})—, —N═C—, —O—N═C—, or —N(R^{N3})—N═C—, wherein R^{N3} is independently hydrogen or C₁₋₆alkyl.

In some embodiments, ZZ is —C(O)N(R^{N3})—, —N(R^{N3})C(O)—, —C(O)O—, —OC(O)—, —OC(O)N(R^{N3})—, —N(R^{N3})C(O)O—, —N(R^{N3})C(O)N(R^{N3})—, —S(O)₂N(R^{N3})—, or —N(R^{N3})S(O)₂—, wherein R^{N3} is independently hydrogen or C₁₋₆alkyl.

In some embodiments, ZZ is —C(O)N(R^{N3})— or —N(R^{N3})C(O)—, wherein R^{N3} is independently hydrogen or C₁₋₆alkyl.

In some embodiments, ZZ is —C(O)O— or —OC(O)—.

In some embodiments, ZZ is —OC(O)N(R^{N3})—, —N(R^{N3})C(O)O—, or —N(R^{N3})C(O)N(R^{N3})—, wherein R^{N3} is independently hydrogen or C₁₋₆alkyl.

In some embodiments, ZZ is —N(R^{N3})C(O)N(R^{N3})—, wherein R^{N3} is independently hydrogen or C₁₋₆alkyl.

In some embodiments, ZZ is —OC(O)N(R^{N3})— or —N(R^{N3})C(O)O—, wherein R^{N3} is independently hydrogen or C₁₋₆alkyl.

In some embodiments, ZZ is —OP(O)(OH)O—, —OP(S)(OH)O—, —OP(O)(OH)—, —OP(S)(OH)—, —P(O)(OH)O—, or —P(S)(OH)O—.

In some embodiments, ZZ is —OP(O)(OH)O— or —OP(S)(OH)O—.

In some embodiments, ZZ is —OP(S)(OH)O—.

In some embodiments, ZZ is —OP(O)(OH)O—.

In some embodiments, ZZ is —OP(O)(OH)—, —OP(S)(OH)—, —P(O)(OH)O—, or —P(S)(OH)O—.

In some embodiments, ZZ is —OP(S)(OH)— or —P(S)(OH)O—.

In some embodiments, ZZ is —OP(O)(OH)— or —P(O)(OH)O—.

### Species of Formula (X)

In another embodiment, the compound of Formula (X) is selected from the group consisting of,

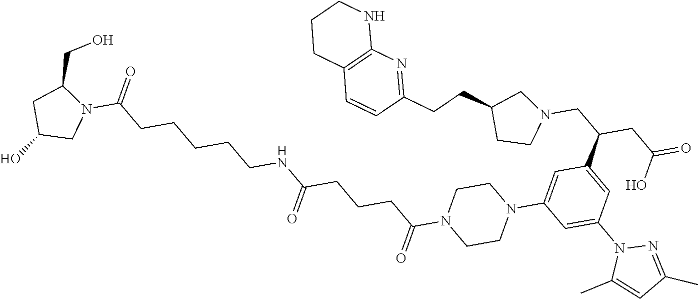 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCCCC(=O)N4C[C@H](O)C[C@H]4CO)CC3)c2)n1]

(S)-3-(3-(3,5-dimethyl-1H-pyrazol-1-yl)-5-(4-(5-((6-((2S,4R)-4-hydroxy-2-(hydroxymethyl)pyrrolidin-
1-yl)-6-oxohexyl)amino)-5-oxopentanoyl)piperazin-1-yl)phenyl)-4-((R)-3-(2-(5,6,7,8-tetrahydro-1,8-
naphthyridin-2-yl)ethyl)pyrrolidin-1-yl)butanoic acid;

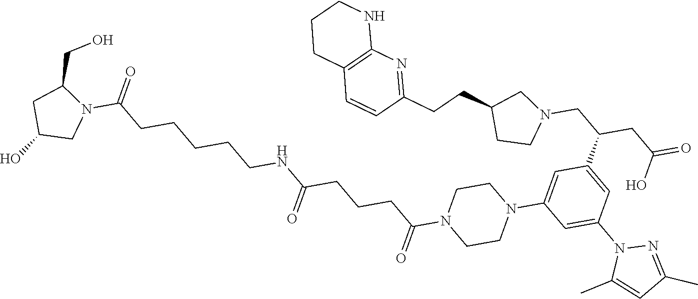 [SMILES: Cc1cc(C)n(-c2cc([C@@H](CC(=O)O)CN3CC[C@@H](CCc4ccc5c(n4)NCCC5)C3)cc(N3CCN(C(=O)CCCC(=O)NCCCCCC(=O)N4C[C@H](O)C[C@H]4CO)CC3)c2)n1]

(R)-3-(3-(3,5-dimethyl-1H-pyrazol-1-yl)-5-(4-(5-((6-((2S,4R)-4-hydroxy-2-(hydroxymethyl)pyrrolidin-
1-yl)-6-oxohexyl)amino)-5-oxopentanoyl)piperazin-1-yl)phenyl)-4-((R)-3-(2-(5,6,7,8-tetrahydro-1,8-
naphthyridin-2-yl)ethyl)pyrrolidin-1-yl)butanoic acid;

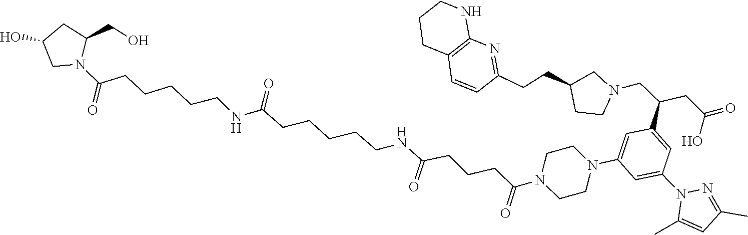 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCCCC(=O)NCCCCCC(=O)N4C[C@H](O)C[C@H]4CO)CC3)c2)n1]

(S)-3-(3-(3,5-dimethyl-1H-pyrazol-1-yl)-5-(4-(5-((6-((6-((2S,4R)-4-hydroxy-2-
(hydroxymethyl)pyrrolidin-1-yl)-6-oxohexyl)amino)-6-oxohexyl)amino)-5-oxopentanoyl)piperazin-1-
yl)phenyl)-4-((R)-3-(2-(5,6,7,8-tetrahydro-1,8-naphthyridin-2-yl)ethyl)pyrrolidin-1-yl)butanoic acid;

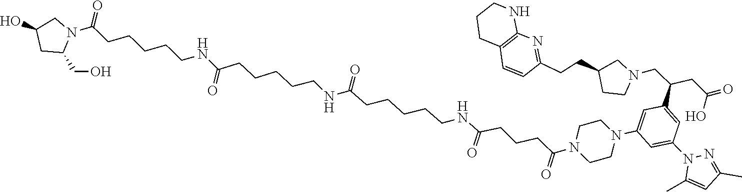 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCCCC(=O)NCCCCCC(=O)NCCCCCC(=O)N4C[C@H](O)C[C@H]4CO)CC3)c2)n1]

(S)-3-(3-(3,5-dimethyl-1H-pyrazol-1-yl)-5-(4-(5-((6-((6-((6-((2S,4R)-4-hydroxy-2-
(hydroxymethyl)pyrrolidin-1-yl)-6-oxohexyl)amino)-6-oxohexyl)amino)-6-oxohexyl)amino)-5-
oxopentanoyl)piperazin-1-yl)phenyl)-4-((R)-3-(2-(5,6,7,8-tetrahydro-1,8-naphthyridin-2-
yl)ethyl)pyrrolidin-1-yl)butanoic acid;

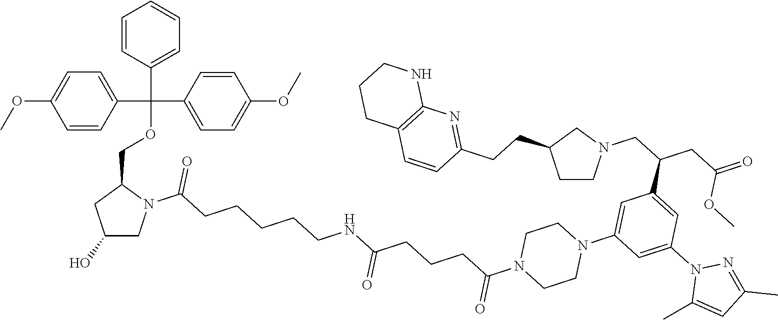

methyl (S)-3-(3-(4-(5-((6-((2S,4R)-2-((bis(4-methoxyphenyl)(phenyl)methoxy)methyl)-4-
hydroxypyrrolidin-1-yl)-6-oxohexyl)amino)-5-oxopentanoyl)piperazin-1-yl)-5-(3,5-dimethyl-1H-
pyrazol-1-yl)phenyl)-4-((R)-3-(2-(5,6,7,8-tetrahydro-1,8-naphthyridin-2-yl)ethyl)pyrrolidin-1-
yl)butanoate;

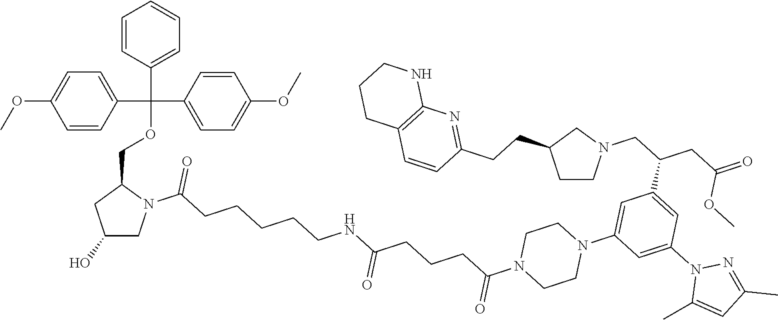

methyl (R)-3-(3-(4-(5-((6-((2S,4R)-2-((bis(4-methoxyphenyl)(phenyl)methoxy)methyl)-4-
hydroxypyrrolidin-1-yl)-6-oxohexyl)amino)-5-oxopentanoyl)piperazin-1-yl)-5-(3,5-dimethyl-1H-
pyrazol-1-yl)phenyl)-4-((R)-3-(2-(5,6,7,8-tetrahydro-1,8-naphthyridin-2-yl)ethyl)pyrrolidin-1-
yl)butanoate

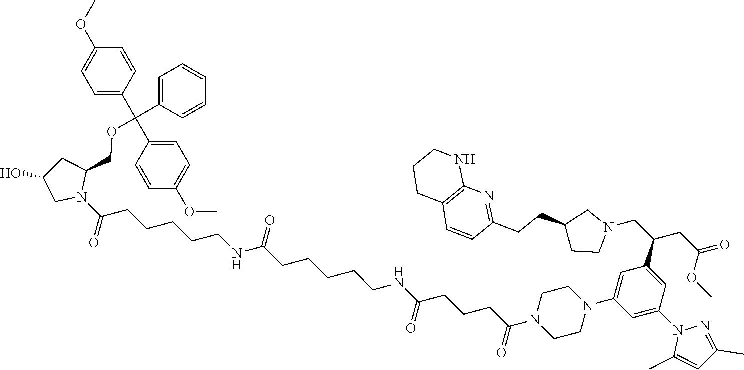 [SMILES: COC(=O)C[C@H](CN1CC[C@@H](CCc2ccc3c(n2)NCCC3)C1)c1cc(N2CCN(C(=O)CCCC(=O)NCCCCCC(=O)NCCCCCC(=O)N3C[C@H](O)C[C@H]3COC(c3ccccc3)(c3ccc(OC)cc3)c3ccc(OC)cc3)CC2)cc(-n2nc(C)cc2C)c1]

methyl (S)-3-(3-(4-(5-((6-((6-((2S,4R)-2-((bis(4-methoxyphenyl)(phenyl)methoxy)methyl)-4-
hydroxypyrrolidin-1-yl)-6-oxohexyl)amino)-6-oxohexyl)amino)-5-oxopentanoyl)piperazin-1-yl)-5-(3,5-
dimethyl-1H-pyrazol-1-yl)phenyl)-4-((R)-3-(2-(5,6,7,8-tetrahydro-1,8-naphthyridin-2-
yl)ethyl)pyrrolidin-1-yl)butanoate;

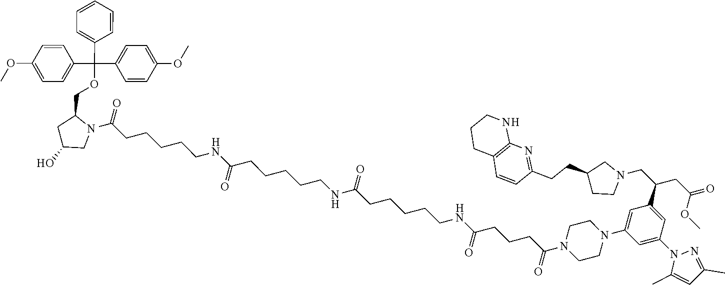

methyl (S)-3-(3-(4-(5-((6-((6-((6-((2S,4R)-2-((bis(4-methoxyphenyl)(phenyl)methoxy)methyl)-4-
hydroxypyrrolidin-1-yl)-6-oxohexyl)amino)-6-oxohexyl)amino)-6-oxohexyl)amino)-5-
oxopentanoyl)piperazin-1-yl)-5-(3,5-dimethyl-1H-pyrazol-1-yl)phenyl)-4-((R)-3-(2-(5,6,7,8-tetrahydro-
1,8-naphthyridin-2-yl)ethyl)pyrrolidin-1-yl)butanoate;

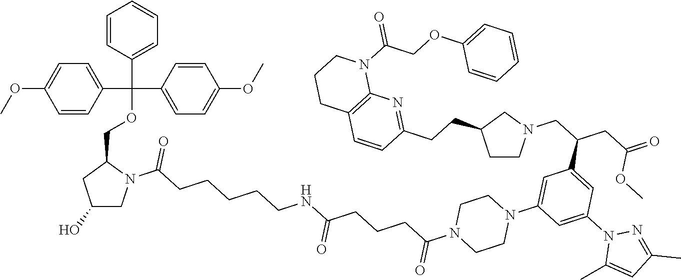

methyl (S)-3-(3-(4-(5-((6-((2S,4R)-2-((bis(4-methoxyphenyl)(phenyl)methoxy)methyl)-4-
hydroxypyrrolidin-1-yl)-6-oxohexyl)amino)-5-oxopentanoyl)piperazin-1-yl)-5-(3,5-dimethyl-1H-
pyrazol-1-yl)phenyl)-4-((R)-3-(2-(8-(2-phenoxyacetyl)-5,6,7,8-tetrahydro-1,8-naphthyridin-2-
yl)ethyl)pyrrolidin-1-yl)butanoate;

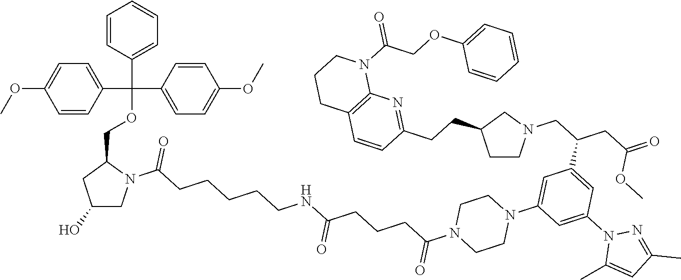

methyl (R)-3-(3-(4-(5-((6-((2S,4R)-2-((bis(4-methoxyphenyl)(phenyl)methoxy)methyl)-4-
hydroxypyrrolidin-1-yl)-6-oxohexyl)amino)-5-oxopentanoyl)piperazin-1-yl)-5-(3,5-dimethyl-1H-
pyrazol-1-yl)phenyl)-4-((R)-3-(2-(8-(2-phenoxyacetyl)-5,6,7,8-tetrahydro-1,8-naphthyridin-2-
yl)ethyl)pyrrolidin-1-yl)butanoate;

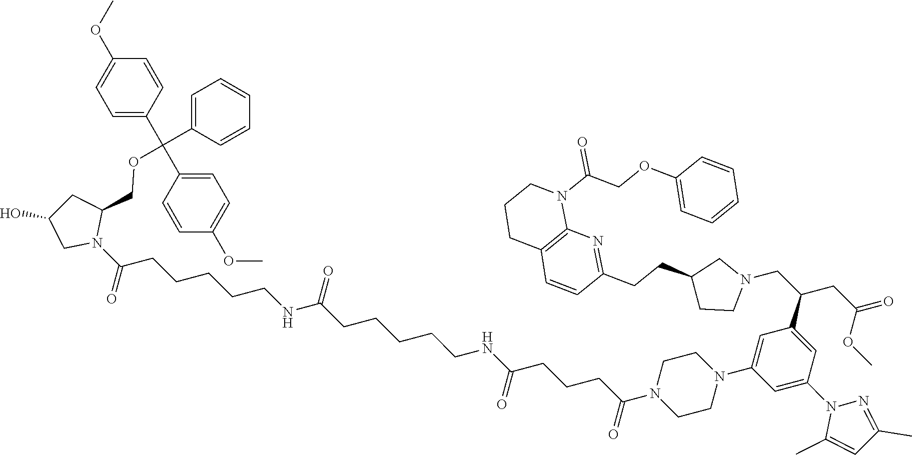 [SMILES: COC(=O)C[C@H](CN1CC[C@@H](CCc2ccc3c(n2)N(C(=O)COc2ccccc2)CCC3)C1)c1cc(N2CCN(C(=O)CCCC(=O)NCCCCCC(=O)NCCCCCC(=O)N3C[C@H](O)C[C@H]3COC(c3ccccc3)(c3ccc(OC)cc3)c3ccc(OC)cc3)CC2)cc(-n2nc(C)cc2C)c1]

methyl (S)-3-(3-(4-(5-((6-((6-((2S,4R)-2-((bis(4-methoxyphenyl)(phenyl)methoxy)methyl)-4-
hydroxypyrrolidin-1-yl)-6-oxohexyl)amino)-6-oxohexyl)amino)-5-oxopentanoyl)piperazin-1-yl)-5-(3,5-
dimethyl-1H-pyrazol-1-yl)phenyl)-4-((R)-3-(2-(8-(2-phenoxyacetyl)-5,6,7,8-tetrahydro-1,8-
naphthyridin-2-yl)ethyl)pyrrolidin-1-yl)butanoate;

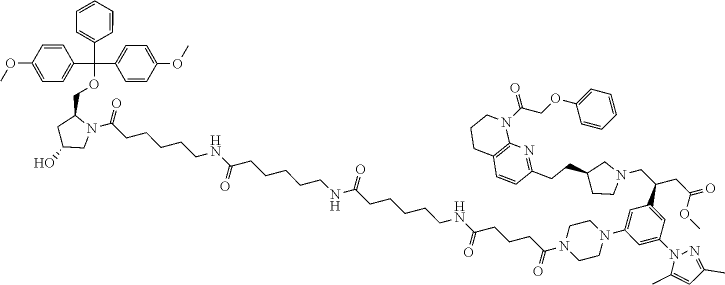

methyl (S)-3-(3-(4-(5-((6-((6-((6-((2S,4R)-2-((bis(4-methoxyphenyl)(phenyl)methoxy)methyl)-
4-hydroxypyrrolidin-1-yl)-6-oxohexyl)amino)-6-oxohexyl)amino)-6-oxohexyl)amino)-5-
oxopentanoyl)piperazin-1-yl)-5-(3,5-dimethyl-1H-pyrazol-1-yl)phenyl)-4-((R)-3-(2-(8-(2-
phenoxyacetyl)-5,6,7,8-tetrahydro-1,8-naphthyridin-2-yl)ethyl)pyrrolidin-1-yl)butanoate;

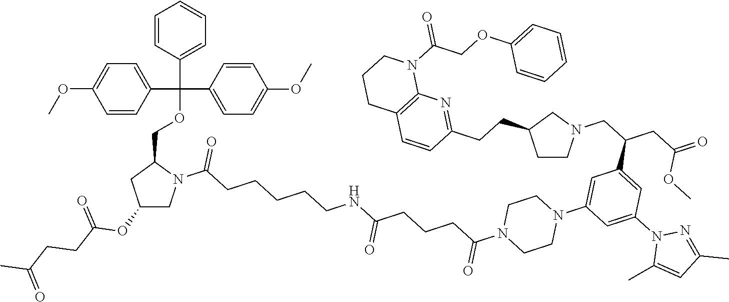 [SMILES: COC(=O)C[C@H](CN1CC[C@@H](CCc2ccc3c(n2)N(C(=O)COc2ccccc2)CCC3)C1)c1cc(N2CCN(C(=O)CCCC(=O)NCCCCCC(=O)N3C[C@H](OC(=O)CCC(C)=O)C[C@H]3COC(c3ccccc3)(c3ccc(OC)cc3)c3ccc(OC)cc3)CC2)cc(-n2nc(C)cc2C)c1]

4-(((3R,5S)-5-((bis(4-methoxyphenyl)(phenyl)methoxy)methyl)-1-(6-(5-(4-(3-(3,5-dimethyl-
1H-pyrazol-1-yl)-5-((S)-4-methoxy-4-oxo-1-((R)-3-(2-(8-(2-phenoxyacetyl)-5,6,7,8-tetrahydro-1,8-
naphthyridin-2-yl)ethyl)pyrrolidin-1-yl)butan-2-yl)phenyl)piperazin-1-yl)-5-
oxopentanamido)hexanoyl)pyrrolidin-3-yl)oxy)-4-oxobutanoic acid;

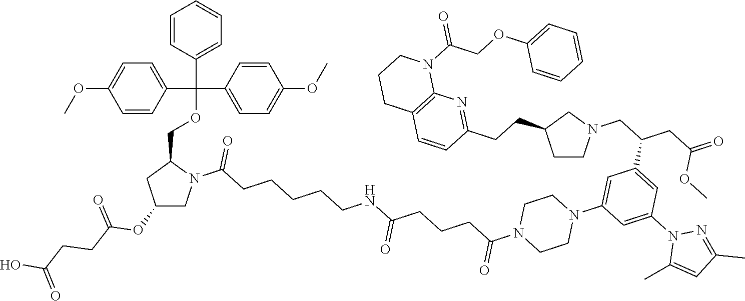 [SMILES: COC(=O)C[C@@H](CN1CC[C@@H](CCc2ccc3c(n2)N(C(=O)COc2ccccc2)CCC3)C1)c1cc(N2CCN(C(=O)CCCC(=O)NCCCCCC(=O)N3C[C@H](OC(=O)CCC(=O)O)C[C@H]3COC(c3ccccc3)(c3ccc(OC)cc3)c3ccc(OC)cc3)CC2)cc(-n2nc(C)cc2C)c1]

4-(((3R,5S)-5-((bis(4-methoxyphenyl)(phenyl)methoxy)methyl)-1-(6-(5-(4-(3-(3,5-dimethyl-
1H-pyrazol-1-yl)-5-((R)-4-methoxy-4-oxo-1-((R)-3-(2-(8-(2-phenoxyacetyl)-5,6,7,8-tetrahydro-1,8-
naphthyridin-2-yl)ethyl)pyrrolidin-1-yl)butan-2-yl)phenyl)piperazin-1-yl)-5-
oxopentanamido)hexanoyl)pyrrolidin-3-yl)oxy)-4-oxobutanoic acid;

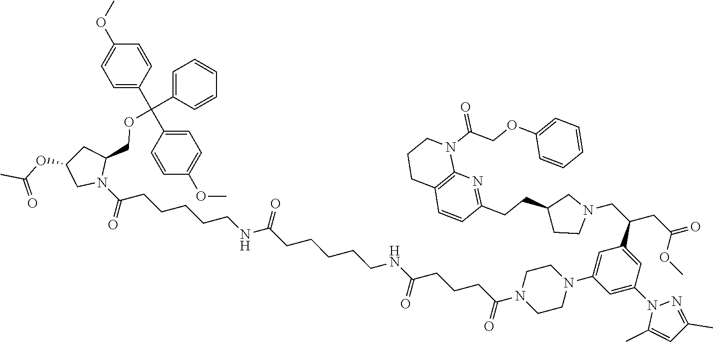 [SMILES: COC(=O)C[C@H](CN1CC[C@@H](CCc2ccc3c(n2)N(C(=O)COc2ccccc2)CCC3)C1)c1cc(N2CCN(C(=O)CCCC(=O)NCCCCCC(=O)NCCCCCC(=O)N3C[C@H](OC(C)=O)C[C@H]3COC(c3ccccc3)(c3ccc(OC)cc3)c3ccc(OC)cc3)CC2)cc(-n2nc(C)cc2C)c1]

4-(((3R,5S)-5-((bis(4-methoxyphenyl)(phenyl)methoxy)methyl)-1-(6-(6-(5-(4-(3-(3,5-dimethyl-1H-
pyrazol-1-yl)-5-((S)-4-methoxy-4-oxo-1-((R)-3-(2-(8-(2-phenoxyacetyl)-5,6,7,8-tetrahydro-1,8-
naphthyridin-2-yl)ethyl)pyrrolidin-1-yl)butan-2-yl)phenyl)piperazin-1-yl)-5-
oxopentanamido)hexanamido)hexanoyl)pyrrolidin-3-yl)oxy)-4-oxobutanoic acid;

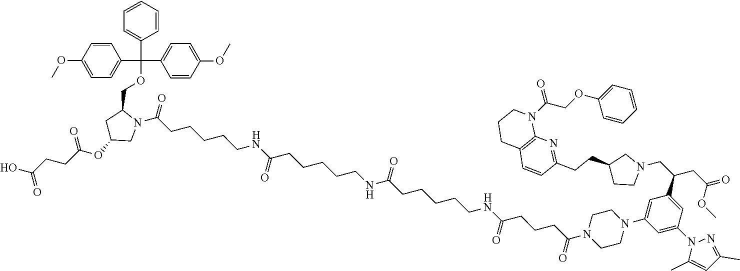 [SMILES: COC(=O)C[C@H](CN1CC[C@@H](CCc2ccc3c(n2)N(C(=O)COc2ccccc2)CCC3)C1)c1cc(N2CCN(C(=O)CCCC(=O)NCCCCCC(=O)NCCCCCC(=O)NCCCCCC(=O)N3C[C@H](OC(=O)CCC(=O)O)C[C@H]3COC(c3ccccc3)(c3ccc(OC)cc3)c3ccc(OC)cc3)CC2)cc(-n2nc(C)cc2C)c1]

4-(((3R,5S)-5-((bis(4-methoxyphenyl)(phenyl)methoxy)methyl)-1-(6-(6-(6-(5-(4-(3-(3,5-dimethyl-1H-
pyrazol-1-yl)-5-((S)-4-methoxy-4-oxo-1-((R)-3-(2-(8-(2-phenoxyacetyl)-5,6,7,8-tetrahydro-1,8-
naphthyridin-2-yl)ethyl)pyrrolidin-1-yl)butan-2-yl)phenyl)piperazin-1-yl)-5-
oxopentanamido)hexanamido)hexanamido)hexanoyl)pyrrolidin-3-yl)oxy)-4-oxobutanoic acid;
and salts thereof.

In another embodiment, the compound of Formula (X) is selected from the group consisting of,

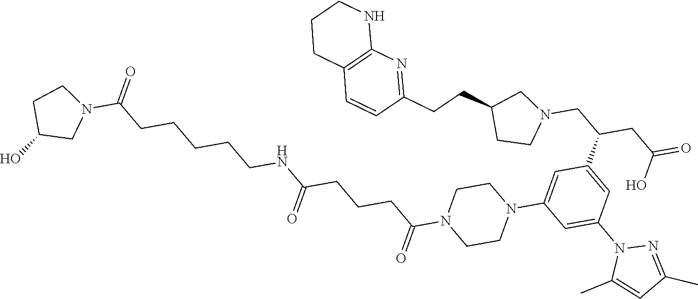 [SMILES: Cc1cc(C)n(-c2cc([C@@H](CC(=O)O)CN3CC[C@@H](CCc4ccc5c(n4)NCCC5)C3)cc(N3CCN(C(=O)CCCC(=O)NCCCCCC(=O)N4CC[C@@H](O)C4)CC3)c2)n1]

(R)-3-(3-(3,5-dimethyl-1H-pyrazol-1-yl)-5-(4-(5-((6-((R)-3-hydroxypyrrolidin-1-yl)-6-oxohexyl)amino)-
5-oxopentanoyl)piperazin-1-yl)phenyl)-4-((R)-3-(2-(5,6,7,8-tetrahydro-1,8-naphthyridin-2-
yl)ethyl)pyrrolidin-1-yl)butanoic acid;

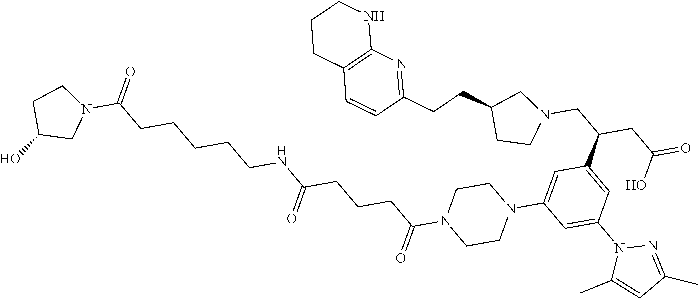 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCCCC(=O)N4CC[C@@H](O)C4)CC3)c2)n1]

(S)-3-(3-(3,5-dimethyl-1H-pyrazol-1-yl)-5-(4-(5-((6-((R)-3-hydroxypyrrolidin-1-yl)-6-oxohexyl)amino)-
5-oxopentanoyl)piperazin-1-yl)phenyl)-4-((R)-3-(2-(5,6,7,8-tetrahydro-1,8-naphthyridin-2-
yl)ethyl)pyrrolidin-1-yl)butanoic acid;

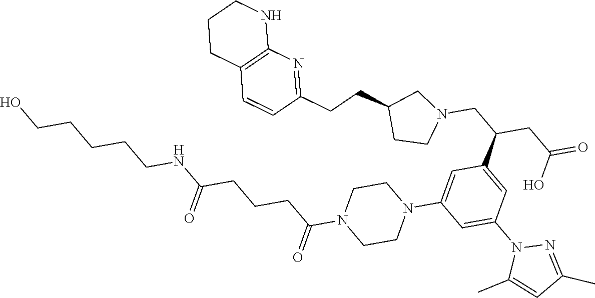 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCCCO)CC3)c2)n1]

(S)-3-(3-(3,5-dimethyl-1H-pyrazol-1-yl)-5-(4-(5-((5-hydroxypentyl)amino)-5-oxopentanoyl)piperazin-1-
yl)phenyl)-4-((R)-3-(2-(5,6,7,8-tetrahydro-1,8-naphthyridin-2-yl)ethyl)pyrrolidin-1-yl)butanoic acid;

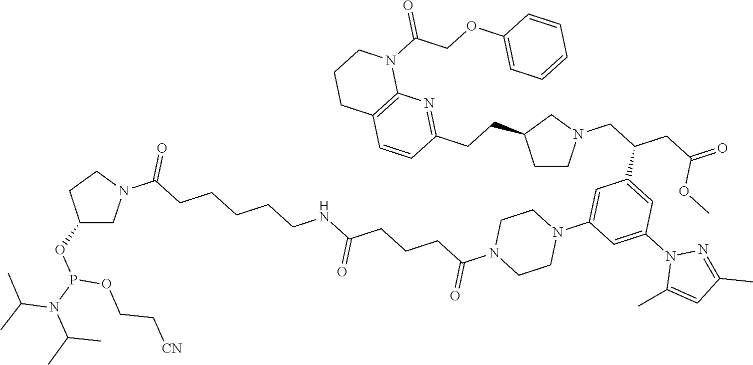 [SMILES: COC(=O)C[C@@H](CN1CC[C@@H](CCc2ccc3c(n2)N(C(=O)COc2ccccc2)CCC3)C1)c1cc(N2CCN(C(=O)CCCC(=O)NCCCCCC(=O)N3CC[C@@H](OP(OCCC#N)N(C(C)C)C(C)C)C3)CC2)cc(-n2nc(C)cc2C)c1]

methyl (3R)-3-(3-(4-(5-((6-((3R)-3-(((2-cyanoethoxy)(diisopropylamino)phosphaneyl)oxy)pyrrolidin-1-
yl)-6-oxohexyl)amino)-5-oxopentanoyl)piperazin-1-yl)-5-(3,5-dimethyl-1H-pyrazol-1-yl)phenyl)-4-
((R)-3-(2-(8-(2-phenoxyacetyl)-5,6,7,8-tetrahydro-1,8-naphthyridin-2-yl)ethyl)pyrrolidin-1-
yl)butanoate;

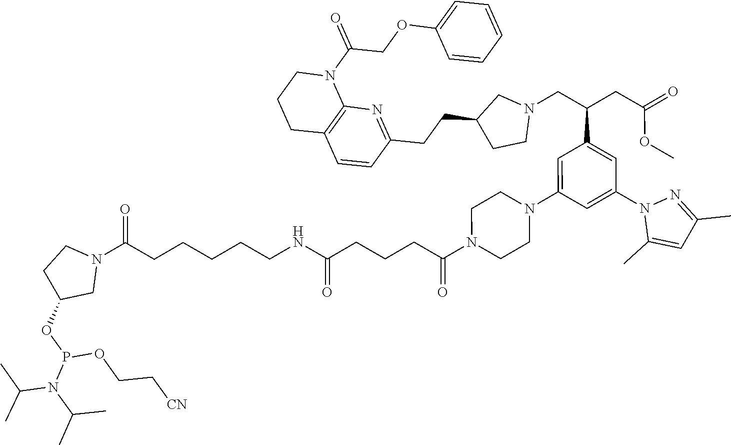 [SMILES: COC(=O)C[C@H](CN1CC[C@@H](CCc2ccc3c(n2)N(C(=O)COc2ccccc2)CCC3)C1)c1cc(N2CCN(C(=O)CCCC(=O)NCCCCCC(=O)N3CC[C@@H](OP(OCCC#N)N(C(C)C)C(C)C)C3)CC2)cc(-n2nc(C)cc2C)c1]

methyl (3S)-3-(3-(4-(5-((6-((3R)-3-(((2-cyanoethoxy)(diisopropylamino)phosphaneyl)oxy)pyrrolidin-1-
yl)-6-oxohexyl)amino)-5-oxopentanoyl)piperazin-1-yl)-5-(3,5-dimethyl-1H-pyrazol-1-yl)phenyl)-4-
((R)-3-(2-(8-(2-phenoxyacetyl)-5,6,7,8-tetrahydro-1,8-naphthyridin-2-yl)ethyl)pyrrolidin-1-
yl)butanoate;

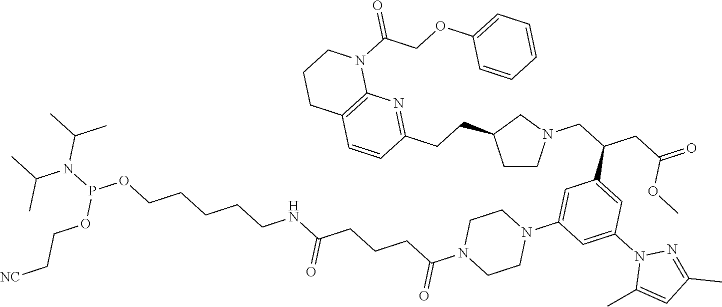 [SMILES: COC(=O)C[C@H](CN1CC[C@@H](CCc2ccc3c(n2)N(C(=O)COc2ccccc2)CCC3)C1)c1cc(N2CCN(C(=O)CCCC(=O)NCCCCCOP(OCCC#N)N(C(C)C)C(C)C)CC2)cc(-n2nc(C)cc2C)c1]

methyl (3S)-3-(3-(4-(5-((5-(((2-cyanoethoxy)(diisopropylamino)phosphaneyl)oxy)pentyl)amino)-5-
oxopentanoyl)piperazin-1-yl)-5-(3,5-dimethyl-1H-pyrazol-1-yl)phenyl)-4-((R)-3-(2-(8-(2-
phenoxyacetyl)-5,6,7,8-tetrahydro-1,8-naphthyridin-2-yl)ethyl)pyrrolidin-1-yl)butanoate;
and salts thereof.

C. Alpha-v-Beta-6 (αvβ6) Integrin Branched Ligands

In another aspect, the present disclosure provides a compound of Formula (V) or (XV):

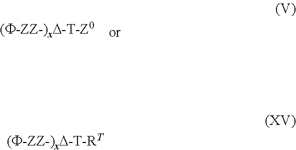

or a salt thereof, wherein
x is 2, 3, 4, 5, 6, 7, or 8;
T is a divalent linking group;
Δ is a branching group;
each ZZ is independently -A′-B′-A′- or a linking group formed by a first reactive pair, wherein
   each A′ is independently a bond, —O—, —S—, or —N(R^{N3})—;
   each B′ is independently a bond, CH₂, C(O), C(S), C(NR^{N3}), —C═N—, S(O), S(O)₂, P(O)(OH), P(S)(OH), or P(S)(SH); and
   each R^{N3} is independently hydrogen or C₁₋₆alkyl, or two R^{N3} within the -A′-B′-A′- group taken together with the atoms to which they are connected from a 4-8 membered heterocyclyl;
Z⁰ is a member of a second reactive pair; and
R^{T} is —R^{T1} or -G⁰-OR^{T1}, wherein
   G⁰ is -D⁰-E⁰-F⁰-, wherein
      D⁰, E⁰, and F⁰ are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups; and
      R^{T1} is hydrogen, a hydroxyl protecting group, a phosphorous coupling group, or -L^{K}-S^{S}, or -L^{L}-oligonucleotide, wherein
         L^{K} is a support linking group;
         S^{S} is a solid support, —OR^{SS} or —N(R^{SS})₂, or hydrogen, wherein each
         R^{SS} is independently hydrogen or C₁₋₆alkyl; and
         L^{L} is an oligonucleotide linking group; and
            each Φ is a compound of the Formula (XII),

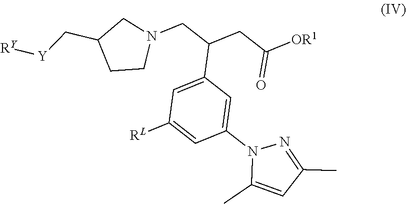

wherein:
Y is O, N(H), S, or CH₂;
R¹ is hydrogen or C₁₋₆alkyl (e.g., methyl);
R^{Y} is

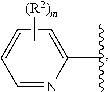

wherein
   m is 0, 1, 2, 3, or 4; and
   each R² is independently R, or two R² groups on adjacent carbon atoms taken together with the atoms to which they are bound form a fused 4-8 membered ring that is optionally substituted by 1, 2, 3 or 4 groups independently selected from group consisting of R and a nitrogen protecting group;
and
R^{L} is —N(R³)(R⁴), —O(R⁵), —S(R⁵), or —R⁵, wherein
   R³ and R⁴ are either
      (i) R³ is hydrogen or C₁₋₆alkyl and R⁴ is R⁵; or
      (ii) R³ and R⁴ taken together with the nitrogen atom to which they are attached form a 4-8 membered monocyclic heterocyclyl group that is substituted with R⁵;
   and
   R⁵ is -L-* wherein L is -L¹-[G-L²]_{q}-G-L³*,
      * is the bond to a ZZ; and
      q is 0 or an integer selected from 1-25; (e.g., 1-20, or 1-15);
      L¹ is a bond or -B-A-;
      each L² is independently -A-B-A-;
      L³ is a bond or -A-B-A-;
      each G is independently -D-E-F-, wherein
         D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
      each A is independently a bond, —O—, —S—, or —N(R^{N})—;
      each B is independently a bond, CH₂, C(O), C(S), C(NR^{N}), S(O), S(O)₂, P(O)(OH), P(S)(OH), or P(S)(SH);
      each R^{N} is independently hydrogen or C₁₋₆alkyl, or two R^{N} within an -A-B-A- group taken together with the atoms to which they are connected from a 4-8 membered heterocyclyl.

Embodiment for the variables of Formula (V), (XII), and (XV) that are the same as Formula (IV) are as described above for Formula (IV).

Embodiment for the variables of Formula (V), (XII), and (XV) that are the same as Formula (X) are as described above for Formula (X). For example, embodiments for the variable ZZ are each as described above for Formula (X); embodiments for the variable R^{T} and L are each as described above for Formula (X).

### T Embodiments, Formula (V) and Formula (XV)

In some embodiments, T is a bond or **-L⁶-G-[L⁵-G¹]_{q1}-L⁴-, wherein ** is the bond to Z⁰ or R^{T};
q1 is 0, 1, 2, 3, 4, 5, 6, 7, 8, 9, or 10;
each L⁴, L⁵, and L⁶ are independently a bond or -A¹-B¹-A¹-;
each G¹ is independently -D¹-E¹-F¹-, wherein D¹, E¹, and F¹ are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, or 3 R groups;
each A¹ is independently a bond, —O—, —S—, or —N(R^{N1})—;
each B¹ is independently a bond, C(O), C(S), C(NR^{N1}), S(O), S(O)₂, P(O)(OH), P(S)(OH), or P(S)(SH);
each R^{N1} is independently hydrogen or C₁₋₆alkyl, or two R^{N1} within an -A¹-B¹-A¹- group taken together with the atoms to which they are connected from a 4-8 membered heterocyclyl.

In some embodiments, T is **-L⁶-G¹-L⁵-G¹-L⁴-, wherein ** is the bond to Z⁰ or R^{T};
L⁴ and L⁶ are independently -A¹-B¹-A¹-;
each L⁵ is a bond or -A¹-B¹-A¹- (e.g., a bond, -B¹-A¹- or -A¹-B¹-; or a bond);
each G¹ is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl (e.g., C₁₋₁₀alkyl or C₂₋₁₀alkenyl);
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In some embodiments, T is **-L⁶-G¹-L⁵-G¹-L⁴-, wherein ** is the bond to Z⁰ or R^{T};
L⁴ and L⁶ are independently -A¹-B¹- or -B¹-A¹-;
each L⁵ is a bond or -A¹-B¹-A¹- (e.g., a bond, -B¹-A¹- or -A¹-B¹-; or a bond);
each G¹ is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl (e.g., C₁₋₁₀alkyl or C₂₋₁₀alkenyl);
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In some embodiments, T is **-B′-G¹-L⁵-G¹-B′-, wherein ** is the bond to Z⁰ or R^{T};
each L⁵ is a bond or -A¹-B¹-A¹- (e.g., a bond, -B¹-A¹- or -A¹-B¹-; or a bond);
each G¹ is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl (e.g., C₁₋₁₀alkyl or C₂₋₁₀alkenyl);
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In some embodiments, T is **-L⁶-G¹-L⁵-G¹-L⁴-, wherein ** is the bond to Z⁰ or R^{T};
L⁴ and L⁶ are independently -A¹-B¹-A¹-;
each L⁵ is a bond or -A¹-B¹-A¹- (e.g., a bond, -B¹-A¹- or -A¹-B¹-; or a bond);
each G¹ is independently C₁₋₁₀alkyl or C₂₋₁₀alkenyl;
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In some embodiments, T is **-L⁶-G¹-L⁵-G¹-L⁴-, wherein ** is the bond to Z⁰ or R^{T};
L⁴ and L⁶ are independently -B¹-A¹- or -A¹-B¹-;
each L⁵ is a bond or -A¹-B¹-A¹- (e.g., a bond, -B¹-A¹- or -A¹-B¹-; or a bond);
each G¹ is independently C₁₋₁₀alkyl or C₂₋₁₀alkenyl;
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In some embodiments, T is **-B′-G¹-L⁵-G¹-B¹-, wherein ** is the bond to Z⁰ or R^{T};
each L⁵ is a bond or -A¹-B¹-A¹- (e.g., a bond, -B¹-A¹- or -A¹-B¹-; or a bond);
each G¹ is independently C₁₋₁₀alkyl or C₂₋₁₀alkenyl;
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In some embodiments, T is **-B¹-C₁₋₁₀alkyl-L⁵-C₁₋₁₀alkyl-B¹-, wherein
** is the bond to Z⁰ or R^{T};
each L⁵ is a bond or -A¹-B¹-A¹- (e.g., a bond, -B¹-A¹- or -A¹-B¹-; or a bond);
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In some embodiments, T is -L⁴-G¹-L⁶-**, wherein ** is the bond to Z⁰ or R^{T};
L⁴ and L⁶ are independently -A¹-B¹-A¹-;
each G¹ is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, or 3 R groups;
each A¹ is independently a bond, —O—, —S—, or —N(R^{N1})—, wherein R^{N1} is hydrogen or C₁₋₆alkyl; and
each B¹ is independently a bond, C(O), C(S), S(O)₂, P(O)(OH), or P(S)(OH).

In some embodiments, T is -L⁴-G¹-L⁶-**, wherein ** is the bond to Z⁰ or R^{T};
L⁴ is —C(O)O— or C(O)N(R^{N1})—, wherein R^{N1} is hydrogen or C₁₋₆alkyl;
L⁶ is —OP(O)(OH)O— or —OP(S)(OH)O—; and
each G¹ is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, or 3 R groups.
   In some embodiments, T is selected from the following, wherein ** is the bond to Z⁰ or R^{T}:
(a) **—C(O)—C₁₋₁₀alkyl-L⁵-C₁₋₁₀alkylC(O)—,
(b) **—C(O)—C₂₋₁₀alkyl-L⁵-C₂₋₁₀alkyl-C(O)—,
(c) **—C(O)—C₄₋₁₀alkyl-L⁵-C₄₋₁₀alkyl-C(O)—,
(d) **—C(O)—C₆₋₁₀alkyl-L⁵-C₆₋₁₀alkyl-C(O)—,
(e) **—C(O)—C₂₋₈alkyl-L⁵-C₂₋₈alkyl-C(O)—,
(f) **—C(O)—C₂₋₆alkyl-L⁵-C₂₋₆alkyl-C(O)—,
(g) **—C(O)—C₂₋₄alkyl-L⁵-C₂₋₄alkyl-C(O)—
(h) **—C(O)—C₂₋₂₀alkyl-C(O)—,
(i) **—C(O)—C₂₋₁₂alkyl-C(O)—,
(j) **—C(O)—C₆₋₂₀alkyl-C(O)—,
(k) **—C(O)—C₆₋₁₂alkyl-C(O)—,
(l) **—C(O)—C₁₀alkyl-C(O)— and
(m) **—C(O)—CH₂CH₂—C(O)—,
   wherein each L⁵ is a bond or -A¹-B¹-A¹- (e.g., a bond, -B¹-A¹- or -A¹-B¹-; or a bond), wherein each A¹ is independently a bond, —O—, —S—, or —N(R^{N1})—, wherein R^{N1} is independently hydrogen or C₁₋₆alkyl; and each B¹ is independently a bond, C(O), C(S), S(O)₂, P(O)(OH), or P(S)(OH).

In some embodiments, T is selected from the following, wherein ** is the bond to Z⁰ or R^{T}:
(n) **—N(H)C(O)—C₂₋₂₀alkyl-C(O)—,
(o) **—N(H)C(O)—C₆₋₂₀alkyl-C(O)—,
(p) **—N(H)C(O)—C₆₋₁₂alkyl-C(O)—,
(q) **—N(H)C(O)—C₁₀alkyl-C(O)—,
(r) **—C(O)—C₂₋₂₀alkyl-C(O)N(H)—,
(s) **—C(O)—C₆₋₂₀alkyl-C(O)N(H)—,
(t) **—C(O)—C₆₋₁₂alkyl-C(O)N(H)—,
(u) **—C(O)—C₁₀alkyl-C(O)N(H)—,
(v) **—N(H)C(O)—C₂₋₂₀alkyl-C(O)N(H)—,
(w) **—N(H)C(O)—C₆₋₂₀alkyl-C(O)N(H)—,
(x) **—N(H)C(O)—C₆₋₁₂alkyl-C(O)N(H)—,
and
(y) **—N(H)C(O)—C₁₀alkyl-C(O)N(H).

In some embodiments, T is -L⁴-G¹-L⁶-**, wherein ** is the bond to Z⁰ or R^{T};
L⁴ is —C(O)O— or C(O)N(R^{N1})—, wherein R^{N1} is hydrogen or C₁₋₆alkyl;
L⁶ is —OP(O)(OH)O— or —OP(S)(OH)O—; and
each G¹ is independently C₃₋₁₀cycloalkyl, 3-10 membered heterocyclyl, aryl, or heteroaryl, each of which is optionally substituted with one R group.

In some embodiments, T is -L⁴-G¹-L⁶-**, wherein ** is the bond to Z⁰ or R^{T};
L⁴ is —C(O)O— or C(O)N(R^{N1})—, wherein R^{N1} is hydrogen or C₁₋₆alkyl;
L⁶ is —OP(O)(OH)O— or —OP(S)(OH)O—; and
each G¹ is independently C₃₋₁₀cycloalkyl or 3-10 membered heterocyclyl.

In some embodiments, T is -L⁴-G¹-L⁶-**, wherein ** is the bond to Z⁰ or R^{T};
L⁴ is —C(O)O— or C(O)N(R^{N1})—, wherein R^{N1} is hydrogen or C₁₋₆alkyl;
L⁶ is —OP(O)(OH)O— or —OP(S)(OH)O—; and
each G¹ is independently C₃₋₁₀cycloalkyl or 3-10 membered heterocyclyl.

In some embodiments, T is

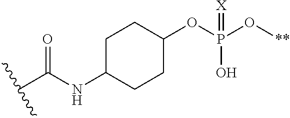

wherein ** is the bond to Z⁰ or R^{T}; and X is O or S (e.g., S).

In some embodiments, T is **-L⁶-[G⁵-O]_{q5}-G⁵-L⁴-, wherein ** is the bond to Z⁰ or R^{T};
q5 is an integer selected from 1 to 20;
L⁴ and L⁶ are independently -A¹-B¹-A¹-, wherein
   each A¹ is independently a bond, —O—, —S—, or —N(R^{N1})—, wherein R^{N1} is hydrogen or C₁₋₆alkyl;
   each B¹ is independently a bond, C(O), C(S), S(O)₂, P(O)(OH), or P(S)(OH);
each G⁵ is independently C₁₋₁₀alkyl; and
each L¹⁰ is —O—.

In some embodiments, T is **—C(O)—[CH₂CH₂—O]_{q5}-G⁵-L⁴-, wherein ** is the bond to Z⁰ or R^{T};
q5 is an integer selected from 1 to 20;
L⁴ is -A¹-B¹-A¹-, wherein
   each A¹ is independently a bond, —O—, —S—, or —N(R^{N1})—, wherein R^{N1} is hydrogen or C₁₋₆alkyl;
   each B¹ is independently a bond, C(O), C(S), S(O)₂, P(O)(OH), or P(S)(OH);
G⁵ is C₁₋₁₀alkyl.

In some embodiments, T is **—C(O)—[CH₂CH₂—O]_{q5}-G⁵-L⁴-, wherein ** is the bond to Z⁰ or R^{T};
q5 is an integer selected from 1 to 20;
L⁴ is -A¹-B¹ or -B¹-A¹-, wherein
   each A¹ is independently —O— or —N(H)—,
   each B¹ is independently C(O),
G⁵ is C₁₋₁₀alkyl (e.g., C₂₋₁₀alkyl or C₂₋₆alkyl).

In some embodiments, T is **—C(O)—[CH₂CH₂—O]_{q5}—C₂₋₁₀alkyl-C(O)N(H)—, wherein ** is the bond to Z⁰ or R^{T}, wherein q5 is an integer selected from 1 to 20 (e.g., 1 to 10, or 2 to 10; or 2-8; or 1; or 2; or 3; or 4.) In some embodiments, Formulae (V) and (XV) are according to one of Formulae (Va) through (Vc) and (XVa) through (XVc), respectively:

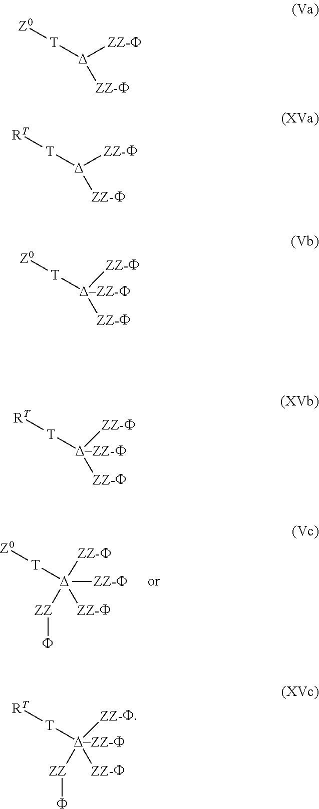

### Δ Embodiments, Formula (V) and Formula (XV)

In some embodiments, Δ is

#-[G²-L⁷]_{q2}-* or #-G³-([L⁷-G⁴]_{q3}-*)_{y},
wherein
# is the bond to T;
y is 1, 2, 3, 4, or 5;
q2 is 1, 2, 3, 4, 5, 6, 7, or 8;
q3 is 0, 1, 2, 3, 4, 5, 6, 7, or 8;
each G², G³, and G⁴ is independently -D²-E²-F²-, wherein
   D², E², and F² are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, 4, or 5 R groups, and
   wherein each G² and G⁴ optionally contains at least one bond to a ZZ (e.g., one bond to a ZZ);
or G³ is N and y is 2;
each L⁷ is independently -A²-B²-A²-; wherein
   each A² is independently a bond, —O—, —S—, or —N(R^{N2})—;
   each B² is independently a bond, C(O), C(S), C(NR^{N2}), S(O), S(O)₂, P(O)(OH), P(S)(OH), or P(S)(SH); and
   each R^{N2} is independently hydrogen, C₁₋₆alkyl, a bond to a ZZ, or two R^{N2} within an -A²-B²-A²- group taken together with the atoms to which they are connected from a 4-8 membered heterocyclyl;
and
each R^{B} is independently halogen, cyano, azido, nitro, —N(R¹⁰)₂, —O(R¹⁰), —S(R¹⁰), —C(O)OR¹⁰, —C(O)R¹⁰, —C(O)N(R¹⁰)₂, —C(NR¹⁰)OR¹⁰, —C(NR¹⁰)R¹⁰, —C(NR¹⁰)N(R¹⁰)₂, —C(S)OR¹⁰, —C(S)R¹⁰, —C(S)N(R¹⁰)₂, —S(O)₂R¹⁰, —S(O)₂OR¹⁰, —S(O)₂N(R¹⁰)₂, —N(R¹⁰)C(O)OR¹⁰, —N(R¹⁰)C(O) R¹⁰, —N(R¹⁰)C(O)N(R¹⁰)₂, —N(R¹⁰)S(O)₂R¹⁰, —N(R¹⁰)S(O)₂OR¹⁰, —N(R¹⁰)S(O)₂N(R¹⁰)₂, —OC(O)OR¹⁰, —OC(O)R¹⁰, —OC(O)N(R¹⁰)₂, —OS(O)₂R¹⁰, —OS(O)₂OR¹⁰, —OS(O)₂N(R¹⁰)₂, or —SC(O)R¹⁰, wherein each R¹⁰ is independently hydrogen, C₁₋₁₀alkyl, C₂₋₁₀alkenyl, C₂₋₁₀alkynyl, C₃₋₁₀cycloalkyl, 3-10 membered heterocyclyl, aryl, or heteroaryl. provided that Δ contains x bonds to ZZ.

In some embodiments, Δ is #4-[G²-L⁷]_{q2}-* wherein
# is the bond to T;
q2 is 1, 2, 3, 4, 5, 6, 7, or 8;
q3 is 0, 1, 2, 3, 4, 5, 6, 7, or 8;
each G², G³, and G⁴ is independently -D²-E²-F²-, wherein
   D², E², and F² are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, 4, or 5 R groups, and
   wherein each G² optionally contains at least one bond to ZZ;
each L⁷ is independently -A²-B²-A²-;
each A² is independently a bond, —O—, —S—, or —N(R^{N2})—;
each B² is independently a bond, C(O), C(S), C(NR^{N2}), S(O), S(O)₂, P(O)(OH), P(S)(OH), or P(S)(SH);
each R^{N2} is independently hydrogen, C₁₋₆alkyl, a bond to a ZZ, or two R^{N2} within an -A²-B²-A²- group taken together with the atoms to which they are connected from a 4-8 membered heterocyclyl;
   each R^{B} is independently halogen, cyano, azido, nitro, —N(R¹⁰)₂, —O(R¹⁰), —S(R¹⁰), —C(O)OR¹⁰, —C(O)R¹⁰, —C(O)N(R¹⁰)₂, —C(NR¹⁰)OR¹⁰, —C(NR¹⁰)R¹⁰, —C(NR¹⁰)N(R¹⁰)₂, —C(S)OR¹⁰, —C(S)R¹⁰, —C(S)N(R¹⁰)₂, —S(O)₂R¹⁰, —S(O)₂OR¹⁰, —S(O)₂N(R¹⁰)₂, —N(R¹⁰)C(O)OR¹⁰, —N(R¹⁰)C(O)R¹⁰, —N(R¹⁰)C(O)N(R¹⁰)₂, —N(R¹⁰)S(O)₂R¹⁰, —N(R¹⁰)S(O)₂OR¹⁰, —N(R¹⁰)S(O)₂ N(R¹⁰)₂, —OC(O)OR¹⁰, —OC(O)R¹⁰, —OC(O)N(R¹⁰)₂, —OS(O)₂R¹⁰, —OS(O)₂OR¹⁰, —OS(O)₂N(R¹⁰)₂, or —SC(O)R¹⁰, wherein
      each R¹⁰ is independently hydrogen, C₁₋₁₀alkyl, C₂₋₁₀alkenyl, C₂₋₁₀alkynyl, C₃₋₁₀cycloalkyl, 3-10 membered heterocyclyl, aryl, or heteroaryl.

In one embodiment, -Δ- is #-[G²-L⁷]_{q2}-*, where # is the bond to T and * is a bond to a ZZ group. For example, such embodiments include the following,

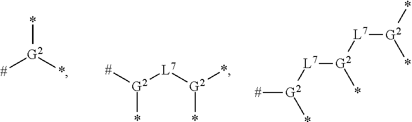

wherein each * is a bond to a ZZ; # is the bond to T,
each G² is independently C₁₋₁₀alkyl, C₂₋₁₀alkenyl, C₃₋₁₀cycloalkyl, 3-10 membered heterocyclyl, aryl, or heteroaryl, each of which is optionally substituted with 1, 2, 3, 4, or 5 R^{B} groups; and each L⁷ is independently -A²-B²-A²-, wherein each A² is independently a bond, —O—, —S—, or —N(R^{N2})—; each B² is independently a bond, C(O), S(O)₂, P(O)(OH), or P(S)(OH).

In one embodiment, each G² is independently C₁₋₁₀alkyl, each optionally substituted with 1 or 2 R^{B} groups. In one embodiment, each G² is independently C₁₋₁₀alkyl.

In one embodiment, each L⁷ is independently -A²-B²-A²-, wherein each A² is independently a bond, —O—, —S—, or —N(R^{N2})—; and each B² is independently a bond, C(O) or S(O)₂, provided that at least one A² is not a bond.

In one embodiment, each L⁷ is independently -A²-B²— or -B²-A²-, wherein each A² is independently, —O—, —S—, or —N(R^{N2})—; and each B² is independently a bond, C(O) or S(O)₂.

For example, such embodiments include each of the following,

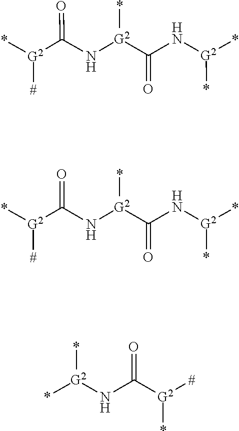

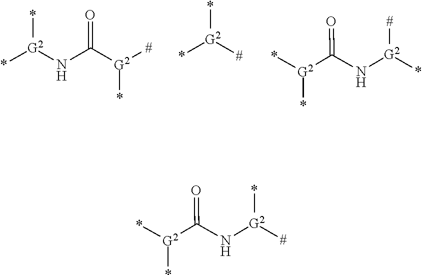

wherein # is the bond to T and each * is a bond to a ZZ group. ; and each G² is independently C₁₋₁₀alkyl.

For example, such embodiments include each of the following, wherein # is the bond to T and each * is a bond to a ZZ;

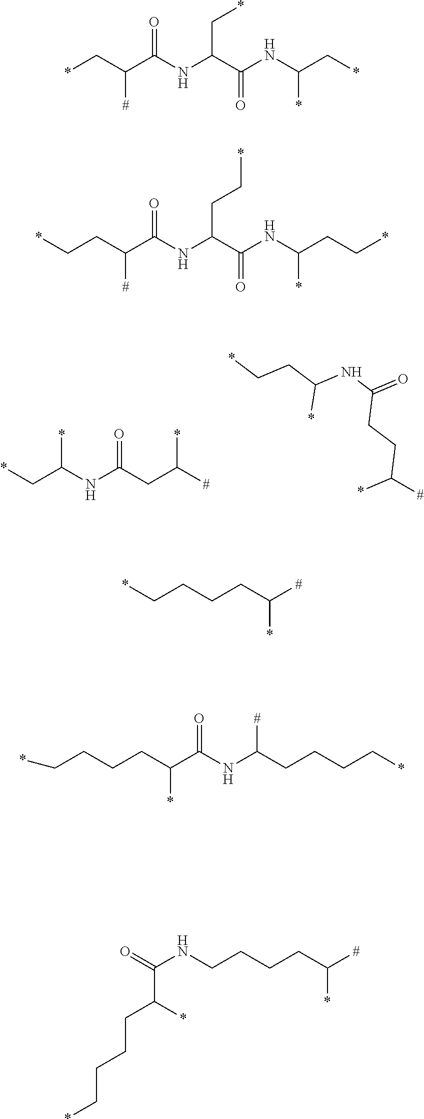

wherein # is the bond to T and each * is a bond to a ZZ group.

In one embodiment, -#-G³-([L⁷-G⁴]_{q3}-*)_{y}, where # is the bond to T and * is a bond to a ZZ group.

For example, such embodiments include each of the following,

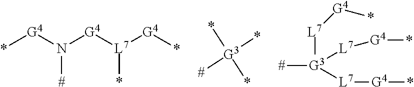

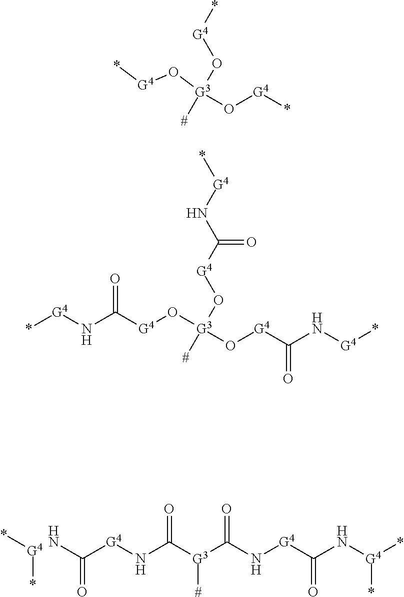

wherein # is the bond to T, each * is a bond to a ZZ group, and
each L⁷ is selected from the group consisting of —O—, —S—, —N(H)—, —C(O)O—, —OC(O)—, —C(O)N(H)—, —OC(O)O—, —N(H)C(O)O—, —OC(O)N(H)—, —OP(O)(OH)O—, or —OP(S)(OH)O—; and each G⁴ is independently -D²-E²-F²-, wherein
   each D² and F² are independently a bond or C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, 4, or 5 R^{B} groups, and
   each E² is independently bond, C₁₋₁₀alkyl, C₂₋₁₀alkenyl, C₃₋₁₀cycloalkyl, 3-10 membered heterocyclyl, aryl, or heteroaryl, each of which is optionally substituted with 1, 2, 3, 4, or 5 R^{B} groups, provided that E² is not a bond with D² and F² are each bonds.

In one embodiment of -#-G³-([L⁷-G⁴]_{q3}-*)_{y},
each L⁷ is selected from the group consisting of —O—, —S—, —N(H)—, —N(H)C(O)—, —C(O)N(H)—, —OP(O)(OH)O—, and —OP(S)(OH)O—;
and each G⁴ is independently -D²-E²-F²-, wherein
   each D² and F² are independently a bond or C₁₋₁₀alkyl;
   each E² is independently C₁₋₁₀alkyl, C₂₋₁₀alkenyl, C₃₋₁₀cycloalkyl, 3-10 membered heterocyclyl, aryl, or heteroaryl, each of which is optionally substituted with 1, 2, 3, 4, or 5 R groups.

In one embodiment of -#-G³-([L⁷-G⁴]_{q3}-*)_{y},
each L⁷ is selected from the group consisting of —O—, —S—, —N(H)—, —N(H)C(O)—, —C(O)N(H)—, —OP(O)(OH)O—, and —OP(S)(OH)O—;
and each G⁴ is independently C₁₋₁₀alkyl, C₂₋₁₀alkenyl, C₃₋₁₀cycloalkyl, 3-10 membered heterocyclyl, aryl, or heteroaryl, each of which is optionally substituted with 1, 2, 3, 4, or 5 R^{B}.
   In one embodiment of -#-G³-([L⁷-G⁴]_{q3}-*)_{y},
each L⁷ is selected from the group consisting of —O—, —S—, —N(H)—, —N(H)C(O)—, —C(O)N(H)—, —OP(O)(OH)O—, and —OP(S)(OH)O—;
and each G⁴ is independently C₁₋₁₀alkyl which is optionally substituted with 1 or, 2 5 R^{B} groups.
   In one embodiment of -#-G³-([L⁷-G⁴]_{q3}-*)_{y}
each L⁷ is selected from the group consisting of —O—, —S—, —N(H)—, —N(H)C(O)—, C(O)N(H)—, —OP(O)(OH)O—, and —OP(S)(OH)O—;
and each G⁴ is independently C₁₋₁₀alkyl.
   In one embodiment of -#-G³-([L⁷-G⁴]_{q3}-*)_{y},
each L⁷ is selected from the group consisting of —N(H)C(O)— and C(O)N(H)—;
and each G⁴ is independently C₁₋₁₀alkyl.

For example, such embodiments include each of the following, wherein each * is a bond to a ZZ;

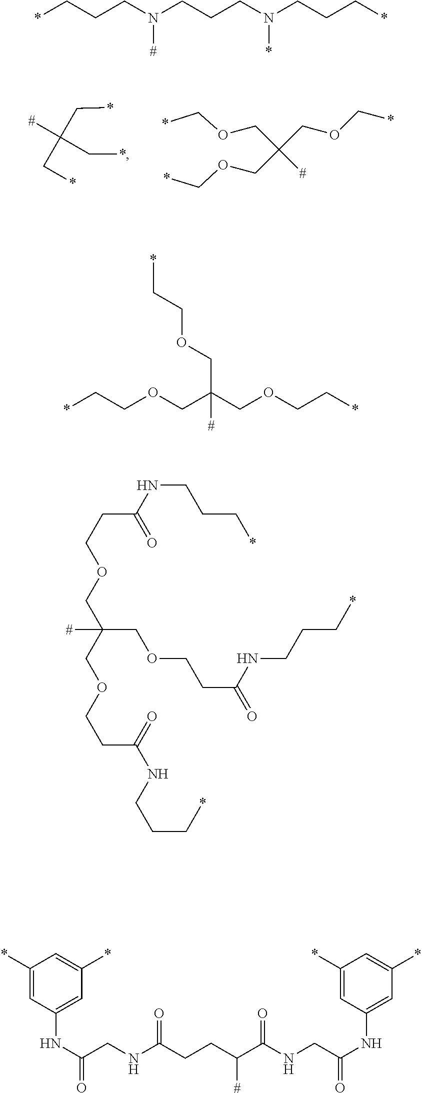

wherein # is the bond to T and each * is a bond to a ZZ group.

### ZZ Embodiments, Formulae (V) and (Va) Through (Vc) and Formulae (XV) and (XVa) Through (XVc)

In embodiments of Formulae (V) and (Va) through (Vc) and Formulae (XV) and (XVa) through (XVc), ZZ is a linking group formed by a reactive pair.

In some embodiments, ZZ comprises a group selected from the group consisting of

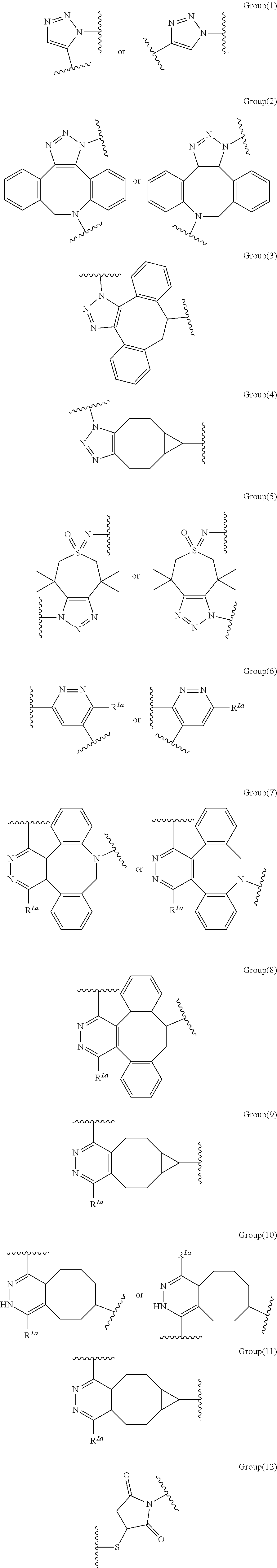

In some embodiments, ZZ comprises Group(1). In some embodiments, ZZ comprises Group(2). In some embodiments, ZZ comprises Group(3). In some embodiments, ZZ comprises Group(4). In some embodiments, ZZ comprises Group(5). In some embodiments, ZZ comprises Group(6). In some embodiments, ZZ comprises Group(7). In some embodiments, ZZ comprises Group(8). In some embodiments, ZZ comprises Group(9). In some embodiments, ZZ comprises Group(10). In some embodiments, ZZ comprises Group(11). In some embodiments, ZZ comprises Group(12).

In some embodiments, wherein ZZ is -A′-B′-A′-.

In some embodiments, ZZ is -A′-B′-A′-, wherein
each A′ is independently a bond, —O—, —S—, or —N(R^{N3})—, wherein R^{N3} is independently hydrogen or C₁₋₆alkyl and
each B′ is independently CH₂, C(O), C(S), S(O)₂, P(O)(OH), or P(S)(OH).

In some embodiments, ZZ is CH₂, C(O), C(S), S(O)₂, P(O)(OH), or P(S)(OH).

In some embodiments, ZZ is C(O), C(S), or S(O)₂.

In some embodiments, ZZ is P(O)(OH), or P(S)(OH).

In some embodiments, ZZ is —C(O)—.

In some embodiments, ZZ is -A′-B′- or -B′-A′- wherein
each A′ is independently —O—, —S—, or —N(R^{N3}
each B′ is independently CH₂, C(O), C(S), S(O)₂, P(O)(OH), or P(S)(OH); and
each R^{N3} is independently hydrogen or C₁₋₆alkyl.

In some embodiments, ZZ is -A′-B′- or -B′-A′- wherein
each A′ is independently —O— or —N(R^{N3})—, wherein R^{N3} is independently hydrogen or C₁₋₆ alkyl.
each B′ is independently CH₂, C(O), S(O)₂, P(O)(OH), or P(S)(OH); and
each R^{N3} is independently hydrogen or C₁₋₆alkyl.

In some embodiments, ZZ is —CH₂O—, —OCH₂—, —S—S—, —C═N—, —C═N—O—, —C═N—N(R^{N3})—, —N═C—, —O—N═C—, —N(R^{N3})—N═C—, —C(O)N(R^{N3})—, —N(R^{N3})C(O)—, —C(O)O—, —OC(O)—, —OC(O)N(R^{N3})—, —N(R^{N3})C(O)O—, —N(R^{N3})C(O)N(R^{N3})—, —S(O)₂N(R^{N3})—, —N(R^{N3})S(O)₂—, —OP(O)(OH)O—, —OP(S)(OH)O—, —OP(O)(OH)—, —OP(S)(OH)—, —P(O)(OH)O—, or —P(S)(OH)O—. wherein R^{N3} is independently hydrogen or C₁₋₆alkyl.

In some embodiments, ZZ is —CH₂O— or —OCH₂—.

In some embodiments, ZZ is —S—S—.

In some embodiments, ZZ is —C═N—, —C═N—O—, —C═N—N(R^{N3})—, —N═C—, —O—N═C—, or —N(R^{N3})—N═C—, wherein R^{N3} is independently hydrogen or C₁₋₆alkyl.

In some embodiments, ZZ is —C(O)N(R^{N3})—, —N(R^{N3})C(O)—, —C(O)O—, —OC(O)—, —OC(O)N(R^{N3})—, —N(R^{N3})C(O)O—, —N(R^{N3})C(O)N(R^{N3})—, —S(O)₂N(R^{N3})—, or —N(R^{N3})S(O)₂—, wherein R^{N3} is independently hydrogen or C₁₋₆alkyl.

In some embodiments, ZZ is —C(O)N(R^{N3})— or —N(R^{N3})C(O)—, wherein R^{N3} is independently hydrogen or C₁₋₆alkyl.

In some embodiments, ZZ is —C(O)O— or —OC(O)—.

In some embodiments, ZZ is —OC(O)N(R^{N3})—, —N(R^{N3})C(O)O—, or —N(R^{N3})C(O)N(R^{N3}), wherein R^{N3} is independently hydrogen or C₁₋₆alkyl.

In some embodiments, ZZ is —N(R^{N3})C(O)N(R^{N3})—, wherein R^{N3} is independently hydrogen or C₁₋₆alkyl.

In some embodiments, ZZ is —OC(O)N(R^{N3})— or —N(R^{N3})C(O)O—, wherein R^{N3} is independently hydrogen or C₁₋₆alkyl.

In some embodiments, ZZ is —OP(O)(OH)O—, —OP(S)(OH)O—, —OP(O)(OH)—, —OP(S)(OH)—, —P(O)(OH)O—, or —P(S)(OH)O—.

In some embodiments, ZZ is —OP(O)(OH)O— or —OP(S)(OH)O—.

In some embodiments, ZZ is —OP(S)(OH)O—.

In some embodiments, ZZ is —OP(O)(OH)O—.

In some embodiments, ZZ is —OP(O)(OH)—, —OP(S)(OH)—, —P(O)(OH)O—, or —P(S)(OH)O—.

In some embodiments, ZZ is —OP(S)(OH)— or —P(S)(OH)O—.

In some embodiments, ZZ is —OP(O)(OH)— or —P(O)(OH)O—.

### Z Embodiments, Formulae (V) and (Va) Through (Vc)

In some embodiments of any one of Formulae (V) and (Va) through (Vc), Z⁰ is azido, amino, hydroxy, —N═C═O, —N═C═S, —SR^{Z1}, —C(O)H, —C(O)OR^{Z}, —C(S)OR^{Z}, —CH₂—X, or a Michael acceptor,
wherein R^{Z} is hydrogen or C₁₋₁₀alkyl;
R^{Z} is hydrogen, pyridyl, or benzotriazolyl;
X is a leaving group.

In some embodiments, Z⁰ is COOH.

In some embodiments, Z⁰ is NH₂.

In some embodiments, Z⁰ is N₃.

In some embodiments, Z⁰ is hydroxy.

In some embodiments, Z⁰ is —SH.

In some embodiments, Z⁰ is

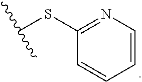 [SMILES: *Sc1ccccn1]

In some embodiments, Z⁰ is a Michael acceptor (e.g., N-maleimido).

In some embodiments, Z⁰ comprises a terminal alkyne,

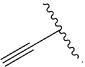 [SMILES: *C#C]

In some embodiments, Z⁰ comprises

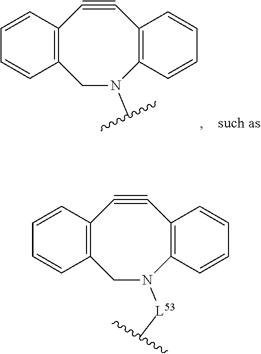

wherein L⁵³ is a bond, —C(O)—, —C(S)—, —S(O)₂—, —C(O)O—, C(O)N(H)—, —S(O)₂N(H)—.

In some embodiments, Z⁰ comprises

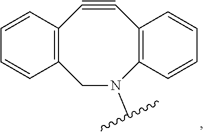 [SMILES: *N1Cc2ccccc2C#Cc2ccccc21]

In some embodiments, Z⁰ comprises

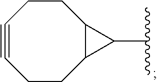 [SMILES: *C1C2CCC#CCCC12]

for example, Z is

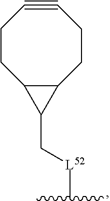

wherein L⁵² is a bond, —O—, —N(H)—, —S—, —C(O)—, —C(S)—, —S(O)₂—, —C(O)O—, —OC(O)—, —C(O)N(H)—, N(H)C(O)—, —OC(O)O—, —OC(O)N(H)—, N(H)C(O)O—, —N(H)C(O)N(H)—, or —S(O)₂N(H)—

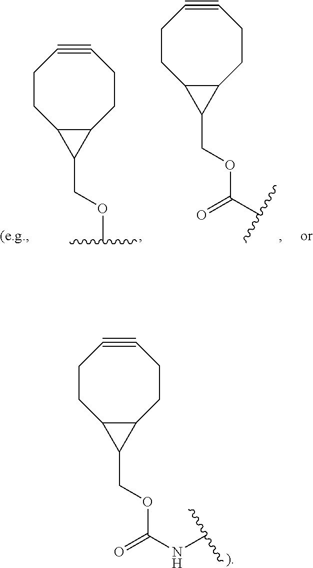 [SMILES: *OCC1C2CCC#CCCC21 ; *C(=O)OCC1C2CCC#CCCC21 ; *NC(=O)OCC1C2CCC#CCCC21]

In some embodiments, Z⁰ comprises

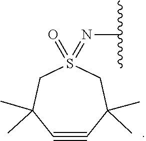

In some embodiments, Z⁰ comprises

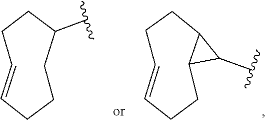 [SMILES: *C1CC/C=C/CCC1 ; *C1C2CC/C=C/CCC12]

such as

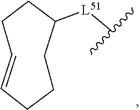

wherein L⁵¹ is a bond, —O—, —N(H)—, —S—, —C(O)—, —C(S)—, —S(O)₂—, —C(O)O—, —OC(O)—, —C(O)N(H)—, N(H)C(O)—, —OC(O)O—, —OC(O)N(H)—, N(H)C(O)O—, —N(H)C(O)N(H)—, —CH₂O—, —CH₂N(H)—, —CH₂S—, —CH₂C(O)O—, —CH₂OC(O)—, —CH₂C(O)N(H)—, —CH₂N(H)C(O)—, —CH₂OC(O)O—, —CH₂OC(O)N(H)—, —CH₂N(H)C(O)O—, —CH₂N(H)C(O)N(H)—, or —S(O)₂N(H)—

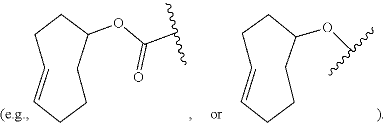 [SMILES: *C(=O)OC1CC/C=C/CCC1 ; *OC1CC/C=C/CCC1]

In some embodiments, Z⁰ comprises

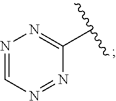 [SMILES: *c1nncnn1]

for example, Z is

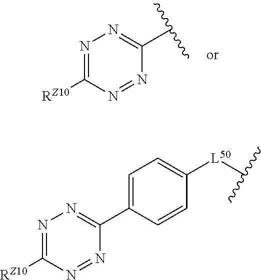

wherein R^{Z10} is hydrogen or C₁₋₁₀alkyl; and L⁵⁰ is a bond, —O—, —N(H)—, —S—, —C(O)—, —C(S)—, —S(O)₂—, —C(O)O—, —OC(O)—, —C(O)N(H)—, N(H)C(O)—, —OC(O)O—, —OC(O)N(H)—, N(H)C(O)O—, —N(H)C(O)N(H)—, —CH₂O—, —CH₂N(H)—, —CH₂S—, —CH₂C(O)O—, —CH₂OC(O)—, —CH₂C(O)N(H)—, —CH₂N(H)C(O)—, —CH₂OC(O)O—, —CH₂OC(O)N(H)—, —CH₂N(H)C(O)O—, —CH₂N(H)C(O)N(H)—, or —S(O)₂N(H)—.

In some embodiments, Z⁰ is an activated ester

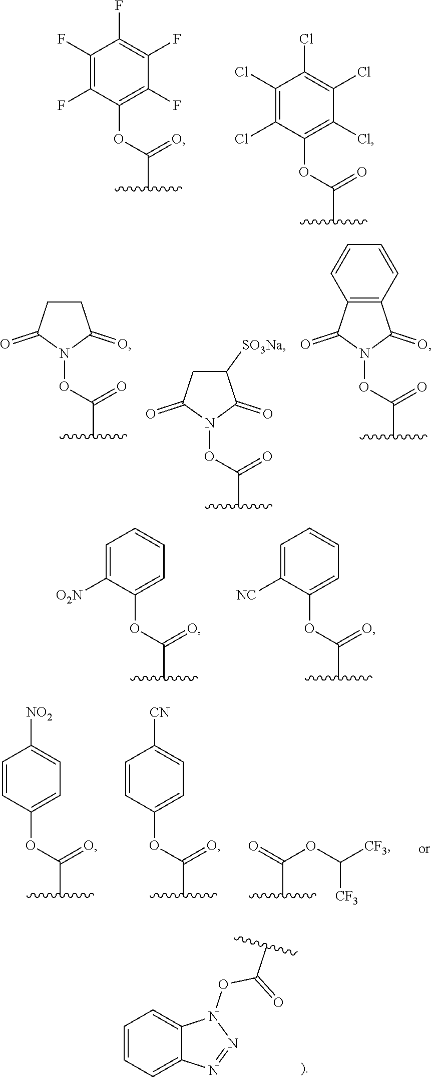

In some embodiments, Z⁰ is

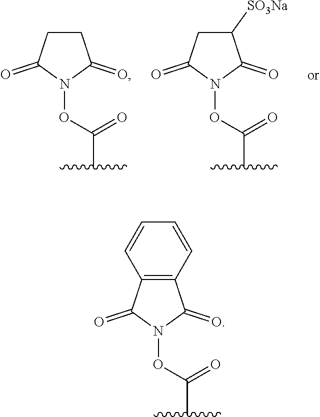

In some embodiments, Z⁰ is

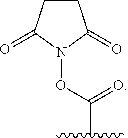 [SMILES: *C(=O)ON1C(=O)CCC1=O]

In some embodiments, Z⁰ is

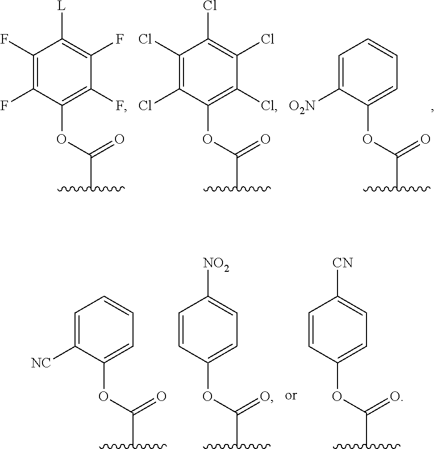

In some embodiments, Z⁰ is

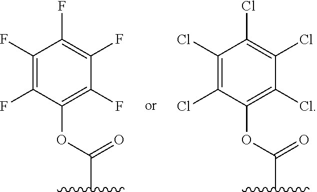 [SMILES: *C(=O)Oc1c(F)c(F)c(F)c(F)c1F ; *C(=O)Oc1c(Cl)c(Cl)c(Cl)c(Cl)c1Cl]

In some embodiments, Z⁰ is

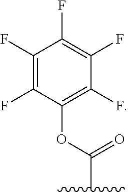 [SMILES: *C(=O)Oc1c(F)c(F)c(F)c(F)c1F]

### R^{T} Embodiments Formula (XV) and (XVa) Through (XVc)

In some embodiments of Formula (XV), R^{T} is R^{T1} is as defined for Formula (X), for example -L^{L}-oligonucleotide.

In some embodiments of Formula (XV), R^{T} is -G⁰-OR^{T1}, wherein R^{T1} is as defined for Formula (X) and G⁰ is selected from:
(l) G⁰ is -D⁰-E⁰-F⁰-, wherein
   D⁰ and F⁰ are independently a bond or C₁₋₁₀alkyl optionally substituted with 1, 2, 3, or 4 R groups; and
   E⁰ is C₃₋₁₀cycloalkyl, 3-10 membered heterocyclyl, aryl, or heteroaryl, each of which is optionally substituted with 1, 2, 3, or 4 R groups;
(m) G⁰ is -D⁰-E⁰-F⁰-, wherein
   D⁰ and F⁰ are independently a bond or C₁₋₁₀alkyl optionally substituted with 1, 2, 3, or 4 R groups; and
   E⁰ is 3-10 membered heterocyclyl optionally substituted with 1, 2, 3, or 4 R groups; and
(n) G⁰ is 3-10 membered heterocyclyl optionally substituted with 1 or 2 R groups; examples include

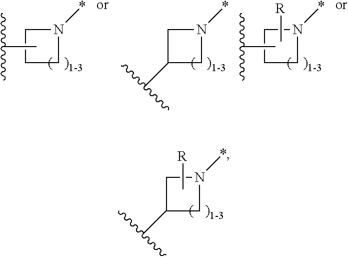

(o) G⁰ is pyrrolidinyl, piperidinyl, piperazinyl, or morpholinyl, each optionally substituted with 1 or 2 R groups; examples include:

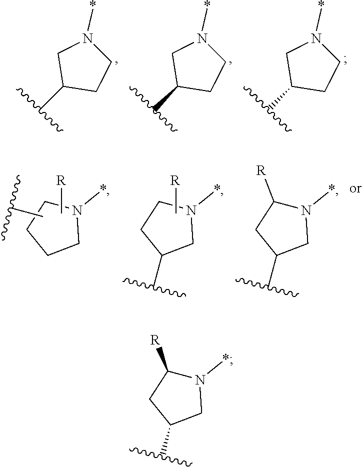

and
(p) G⁰ is 3-10 membered-heterocyclyl-C₁₋₁₀alkyl, optionally substituted with 1, 2, 3, or 4 R groups; examples include,

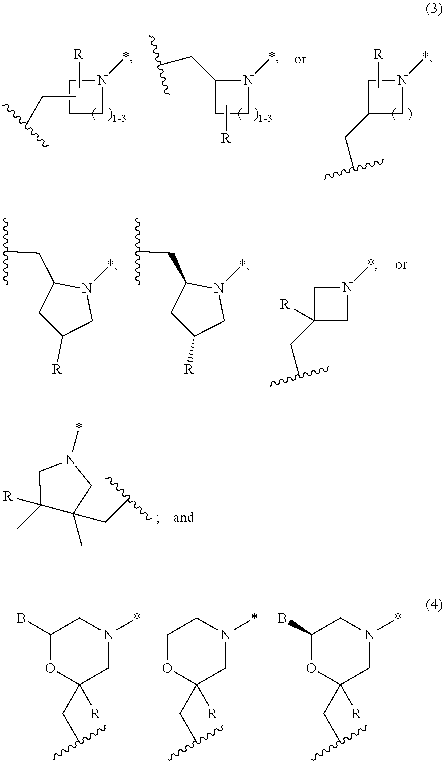

(q) G⁰ is C₁₋₁₀alkyl, C₂₋₁₀alkenyl, C₂₋₁₀alkynyl, each of which is optionally substituted with 1, 2, 3, or 4 R groups (e.g., 1 or 2 R groups);
(r) G⁰ is C₁₋₁₀alkyl or C₂₋₁₀alkenyl, each of which is optionally substituted with 1 or 2 R groups;
(s) G⁰ is C₁₋₁₀alkyl optionally substituted with 1 or 2 R groups;
(t) G⁰ is C₁₋₁₀alkyl, optionally substituted with —O(R^{a}), wherein R^{a} is independently hydrogen, C₁₋₆alkyl, or a hydroxyl protecting group; e.g.,

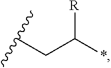

(u) G⁰ is C₁₋₁₀alkyl,
and
(v) G⁰ is absent (a bond);
   wherein * represents the bond to T, the broken bond represents the bond to OR^{T1}, and R is —C₁₋₆alkyl-OR^{a} or —OR^{a}, wherein R^{a} is independently hydrogen, C₁₋₆alkyl, or a hydroxyl protecting group;

### Φ Embodiments

In one embodiment, R^{Y} is defined in the structure of Formula (XII) according to any one of the R^{Y} Embodiments of Formula (IV).

In another embodiment, Formula (XII) has the structure described above for any embodiment of Formula (IV), where the variable Z is replaced by a bond to a ZZ group.

In another embodiment, Formula (XII) has the structure of any one of Formula (XII-a) through (XII-h) and (XII-r):

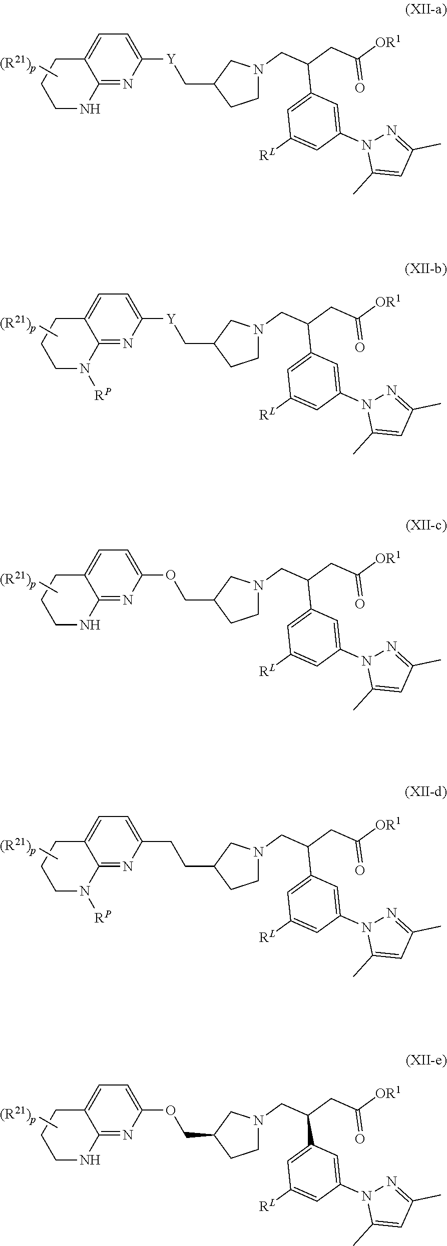

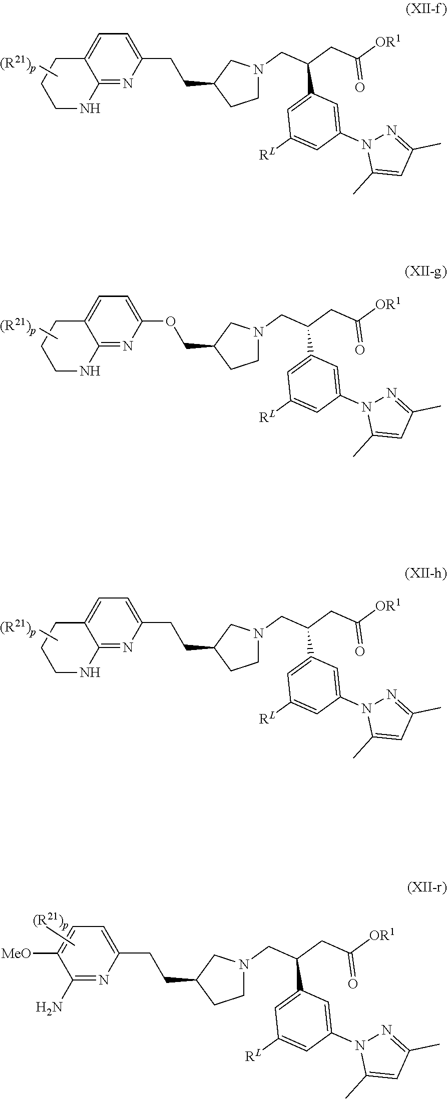

wherein p is 0, 1, 2 or 3;
each R²¹ is independently selected from group consisting of R and a nitrogen protecting group, and R^{P} is a nitrogen protecting group, and
R, R¹, and R^{L} are as defined above for Formula (XII).

### L Embodiments Formula (XII) and (XII-a) Through (XII-h) and (XII-r)

In some embodiments of any one of Formula (XII) and Formulae (XII-a) through (XII-h) and (XII-r), L is -L¹-[G-L²]-G-L³-*, wherein * is the bond to a ZZ group.

In another embodiment, wherein L is -L¹-[G-L²]-G-L³-*, wherein q is 0, 1, 2, 3, 4, or 5.

In another embodiment, L is -L¹-[G-L²]-G-L³-*, wherein q is 0, 1, 2, 3, or 4

In another embodiment, L is -L¹-[G-L²]-G-L³-*, wherein q is 0, 1, 2, or 3.

In another embodiment, L is -L¹-[G-L²]-G-L³-*, wherein q is 0, 1, or 2.

In another embodiment, L is -L¹-[G-L²]-G-L³-*, wherein q is 1, 2, 3, 4, or 5.

In another embodiment, L is -L¹-[G-L²]-G-L³-*, wherein q is 1, 2, 3, or 4.

In another embodiment, L is -L¹-[G-L²]-G-L³-*, wherein q is 1, 2, or 3.

In another embodiment, L is -L¹-[G-L²]-G-L³-*, wherein q is 1 or 2.

In another embodiment, L is -L¹-[G-L²]-G-L³-*, wherein q is 4. In another embodiment, L is -L¹-[G-L²]_{q}-G-L³-*, wherein q is 3. In another embodiment, L is -L¹-[G-L²]-G-L³-*, wherein q is 2.

In another embodiment, L is -L¹-G-L²-G-L³-*. In another embodiment nts, L is -L1-G-L³-*.

In another embodiment, L is -G-L³-*. In another embodiment, L is -L¹-G-*. In another embodiment, L is -G-*.

In some embodiments, each instance of A-B-A- is independently selected from the group consisting of —C(O)O—, —OC(O)—, —C(O)N(R^{N})—, —N(R^{N})C(O)—, —OC(O)O—, —OC(O)N(R^{N})—, —N(R^{N})C(O)O—, —N(R^{N})C(O)N(R^{N})—, —OP(O)(OH)O—, —OP(S)(OH)O—, —O—, and —N(R^{N})—, wherein each R^{N} is independently hydrogen or C₁₋₆alkyl.

In some embodiments, each instance of A-B-A- is independently selected from the group consisting of —C(O)O—, —OC(O)—, —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl.

In some embodiments, each instance of A-B-A- is independently selected from the group consisting of —C(O)N(R^{N})—, —N(R^{N})C(O)—, —OC(O)N(R^{N})—, —N(R^{N})C(O)O—, —N(R^{N})C(O)N(R^{N})—, —O—, and —N(R^{N})—, wherein each R^{N} is independently hydrogen or C₁₋₆alkyl.

In some embodiments, each instance of A-B-A- is independently selected from the group consisting of —C(O)O—, —OC(O)—, —C(O)N(R^{N})—, —N(R^{N})C(O)—, —O—, and —N(R^{N})—, wherein each R^{N} is independently hydrogen or C₁₋₆alkyl.

In some embodiments, each instance of A-B-A- is independently selected from the group consisting of —C(O)N(R^{N})—, —N(R^{N})C(O)—, —O—, and —N(R^{N})—, wherein each R^{N} is independently hydrogen or C₁₋₆alkyl.

In some embodiments,
D and F are each independently a bond, C₁₋₁₀alkyl, C₂₋₁₀alkenyl, or C₂₋₁₀alkynyl, each optionally substituted with 1, 2, 3, or 4 R groups; and
E is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments,
D and F are each independently a bond or C₁₋₁₀alkyl optionally substituted with 1, 2, 3, or 4 R groups; and
E is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments, each G is independently C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups.

In some embodiments, each G is independently C₁₋₁₀alkyl, optionally substituted with 1, 2, or 3 R groups.

In some embodiments, each G is independently C₁₋₁₀alkyl, optionally substituted with 1 or 2 R groups.

In some embodiments, each G is independently C₁₋₁₀alkyl, optionally substituted with one R group

In some embodiments, L is -L¹-G-L³-*, wherein * is the bond to a ZZ group;
G is -D-E-F-, wherein D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-A-;
L³ is a bond or -A-B-A-;
each A is independently a bond, —O—, —S—, or —N(R^{N})—, wherein each R^{N} is independently hydrogen or C₁₋₆alkyl.
each B is independently a bond, CH₂, C(O), S(O)₂, P(O)(OH), or P(S)(OH); and

In some embodiments, L is -L¹-G-L³-*, wherein * is the bond to a ZZ group;
G is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-;
L³ is a bond or -A-B-A-;
each A is independently a bond, —O—, —S—, or —N(R^{N})—, wherein each R^{N} is independently hydrogen or C₁₋₆alkyl; and
each B is independently a bond, CH₂, C(O), S(O)₂, P(O)(OH), or P(S)(OH; and

In some embodiments, L is -L¹-G-*, wherein * is the bond to a ZZ group;
G is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-A-, wherein,
   A is a bond, —O—, —S—, or —N(R^{N})—, wherein each R^{N} is independently hydrogen or C₁₋₆ alkyl; and
   B is a bond, C(O), C(S), S(O)₂, P(O)(OH), or P(S)(OH).

In some embodiments, L is -L¹-G-*, wherein * is the bond to a ZZ group;
G is C₁₋₁₀ alkyl, C₂₋₁₀alkenyl, C₂₋₁₀alkynyl, each of which is optionally substituted with 1 or 2 R groups;
L¹ is -B-A-, wherein
   A is a bond, —O—, —S—, or —N(R^{N})—, each R^{N} is independently hydrogen or C₁₋₆alkyl; and
   B is a bond, C(O), S(O)₂, P(O)(OH), or P(S)(OH).

In some embodiments L is -L¹-G-*, wherein * is the bond to a ZZ group;
G is C₁₋₁₀alkyl or C₂₋₁₀alkenyl, each of which is optionally substituted with 1 or 2 R groups;
L¹ is bond, C(O), S(O)₂, P(O)(OH), or P(S)(OH); and
R^{N} is hydrogen or C₁₋₆alkyl.

In some embodiments, L is

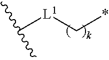

wherein * is the bond to a ZZ group; k is an integer from 1 to 10; L¹ is bond, C(O), C(S), C(NR^{N}), S(O)₂, P(O)(OH), or P(S)(OH); and R^{N} is hydrogen or C₁₋₆alkyl.

In some embodiments, L is

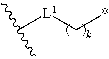

wherein * is the bond to a ZZ group; k is an integer from 1 to 10; L¹ is bond, C(O), P(O)(OH), or P(S)(OH).

In some embodiments, L is

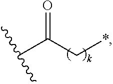

wherein * is the bond to a ZZ group; k is an integer from 1 to 10; or an integer from 2 to 10; or an integer from 3 to 10; or an integer from 4 to 10; or an integer from 5 to 10; or an integer from 5 to 9; or an integer from 5 to 8; or an integer from 5 to 7.

In some embodiments, L is

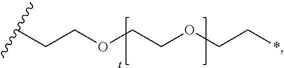

wherein * is the bond to a ZZ group; t is an integer from 0 to 10 (e.g., an integer from 1 to 5; or 1; or 2; or 3).

In some embodiments, L is

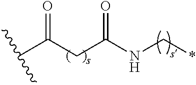

wherein * is the bond to a ZZ group; t is an integer from 0 to 10 (e.g., an integer from 1 to 5 or 1; or 2; or 3); a is an integer from 1 to 3; and s and s′ are each independently an integer from 1 to 24 (e.g., an integer from 1 to 16; an integer from 1 to 10; an integer from 3 to 10; an integer from 3 to 7; or an integer from 4 to 6).

In some embodiments, L is

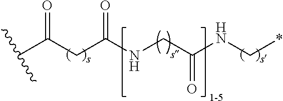

wherein * is the bond to a ZZ group; a is 1, 2 or 3; and each s, s′, and s″ independently is an integer from 1 to 24 (e.g., an integer from 1 to 16, an integer from 1 to 10, an integer from 3 to 10, an integer from 3 to 7, or an integer from 4 to 6).

In some embodiments, L is

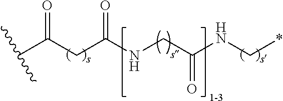

wherein * is the bond to a ZZ group; and s, s′, and s″ are independently is an integer from 1 to 24 (e.g., an integer from 1 to 16, an integer from 1 to 10, an integer from 3 to 10, an integer from 3 to 7, or an integer from 4 to 6).

In some embodiments, L is

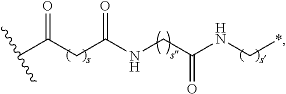

wherein * is the bond to a ZZ group; and each s, s′, and s″ independently is an integer from 1 to 24 (e.g., an integer from 1 to 16, an integer from 1 to 10, an integer from 3 to 10, an integer from 3 to 7, or an integer from 4 to 6).

In some embodiments, L is

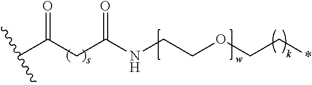

wherein * is the bond to ZZ; s and k are independently is an integer from 1 to 20 (e.g., an integer from 1 to 16, an integer from 1 to 10, an integer from 3 to 10, an integer from 3 to 7 or an integer from 4 to 6); and w is an integer from 1 to 10 (e.g., an integer from 1 to 16, an integer from 1 to 10, an integer from 3 to 10, an integer from 3 to 7 or an integer from 4 to 6).

In some embodiments, L is

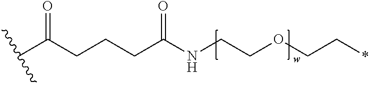

wherein * is the bond to ZZ and w is an integer from 1 to 20 (e.g., an integer from 1 to 16, an integer from 1 to 10, an integer from 3 to 10, an integer from 3 to 7 or an integer from 4 to 6).

### R^{L} Embodiments, Formula (XII) and (XII-a) Through (XII-h) and (XII-r)

In some embodiments of any one of Formula (XII) and Formulae (XII-a) through (XII-h) and (XII-r), R^{L} is —N(R³)(R⁴), wherein R³ is hydrogen or C₁₋₆alkyl and R⁴ is R⁵.

In some embodiments, R^{L} is —N(R³)(R⁴), wherein R^{P3} is hydrogen and R⁴ is R⁵.

In some embodiments, R^{L} is —N(R³)(R⁴), wherein R^{P3} is C₁₋₃alkyl and R⁴ is R⁵.

In some embodiments, R^{L} is —N(R³)(R⁴), wherein R^{P3} is methyl and R⁴ is R⁵.

In some embodiments, R^{L} is —N(R³)(R⁴), wherein R³ and R⁴ taken together with the nitrogen atom to which they are attached form a 4-8 membered monocyclic heterocyclyl group that is substituted with R⁵.

In some embodiments, R^{L} is —N(R³)(R⁴), wherein R³ and R⁴ taken together with the nitrogen atom to which they are attached form a 4-8 membered monocyclic heterocyclyl group that is substituted with R⁵, provided that R³ and R⁴ taken together with the nitrogen atom to which they are attached do not form a morpholino group.

In some embodiments, R^{L} is —N(R³)(R⁴), wherein R³ and R⁴ taken together with the nitrogen atom to which they are attached form a group that is piperidinyl, piperazinyl, pyrrolidinyl, imidazolidinyl, pyrazolidinyl, oxazolidinyl, thiazolidinyl, azetidinyl, pyrrolinyl, imidazolinyl, or pyrazolinyl, each substituted with R⁵.

In some embodiments, R^{L} is

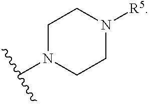

In some embodiments, R^{L} is

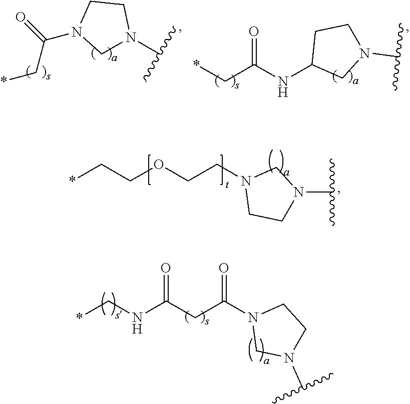

wherein * is a bond to a ZZ group; t is an integer from 0 to 10 (e.g., 0, 1, 2, 3, 4, 5, 6, 7, 8, 9 or 10); a is an integer from 1 to 3 and s and s′ are each independently an integer from 1 to 24 (e.g., an integer from 1 to 16, an integer from 1 to 10, an integer from 3 to 10, an integer from 3 to 7 or an integer from 4 to 6).

In some embodiments, R^{L} is

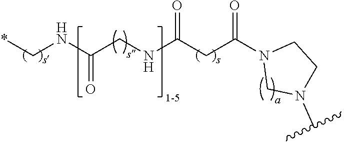

wherein * is a bond to a ZZ group; a is an integer from 1 to 3; and s, s′, and s″ are each independently an integer from 1 to 24 (e.g., an integer from 1 to 16, an integer from 1 to 10, an integer from 3 to 10, an integer from 3 to 7 or an integer from 4 to 6).

In some embodiments, R^{L} is

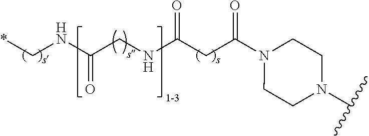

wherein * is a bond to a ZZ group; and s, s′, and s″ are independently is an integer from 1 to 24 (e.g., an integer from 1 to 16, an integer from 1 to 10, an integer from 3 to 10, an integer from 3 to 7 or an integer from 4 to 6.

In some embodiments, R^{L} is

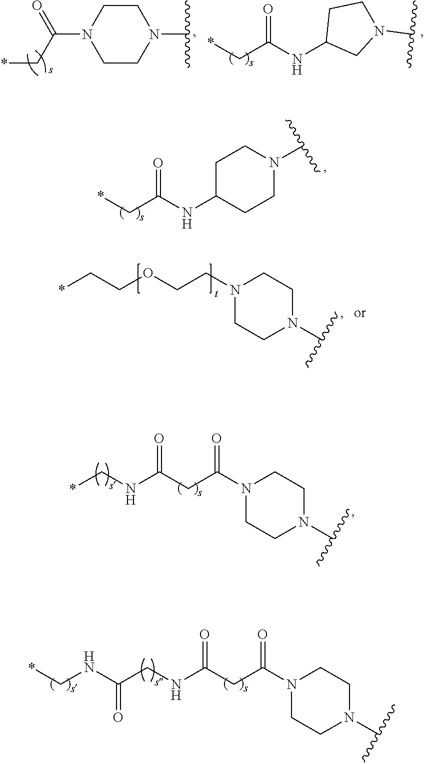

wherein * is a bond to a ZZ group; s, s′, and s″ are independently an integer from 1 to 24 (e.g., an integer from 1 to 16, an integer from 1 to 10, an integer from 3 to 10, an integer from 3 to 7 or an integer from 4 to 6); and t is an integer from 1 to 10 (e.g., an integer from 1 to 8, an integer from 1 to 5, an integer from 1 to 3, or 1, or 2, or 3).

In some embodiments, R^{L} is

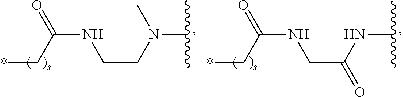

wherein * is a bond to a ZZ group; s is an integer from 1 to 24 (e.g., an integer from 1 to 16, an integer from 1 to 10, an integer from 3 to 10, an integer from 3 to 7 or an integer from 4 to 6).

In some embodiments, R^{L} is —O(R⁵).

In some embodiments, R^{L} is

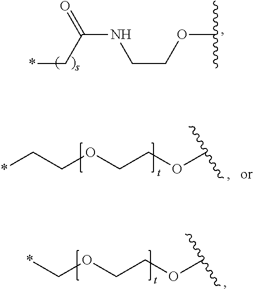

wherein * is a bond to a ZZ group; s is an integer from 1 to 24 (e.g., an integer from 1 to 16, an integer from 1 to 10, an integer from 3 to 10, an integer from 3 to 7 or an integer from 4 to 6); and t is an integer from 0 to 10 (e.g., an integer from 1 to 8, an integer from 1 to 5, an integer from 1 to 3, or 1, or 2, or 3).

In some embodiments, R^{L} is —R⁵.

In some embodiments, R^{L} is

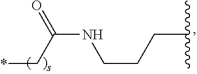

wherein * is a bond to a ZZ group; s is 1-24 (e.g., an integer from 1 to 16, an integer from 1 to 10, an integer from 3 to 10, an integer from 3 to 7 or an integer from 4 to 6).

In some embodiments, R^{L} is

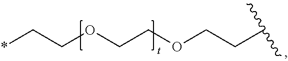

wherein * is a bond to a ZZ group; t is 0 to 10 (e.g., 1-5; or 1-3; or 1; or 2; or 3).

In some embodiments, R^{L} is

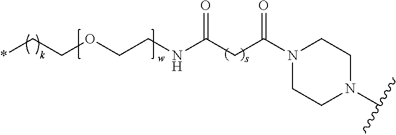

wherein * is a bond to a ZZ group; s and k are independently is an integer from 1 to 20 (e.g., an integer from 1 to 16, an integer from 1 to 10, an integer from 3 to 10, an integer from 3 to 7 or an integer from 4 to 6); and w is an integer from 1 to 10 (e.g., an integer from 1 to 16, an integer from 1 to 10, an integer from 3 to 10, an integer from 3 to 7 or an integer from 4 to 6).

In some embodiments, R^{L} is

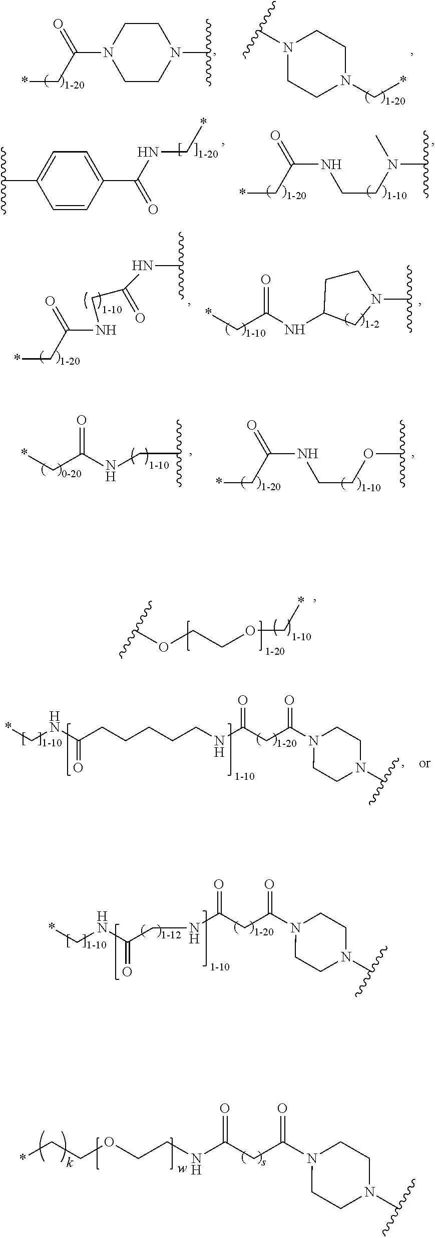

wherein * is a bond to a ZZ group.

In some embodiments, R^{L} is

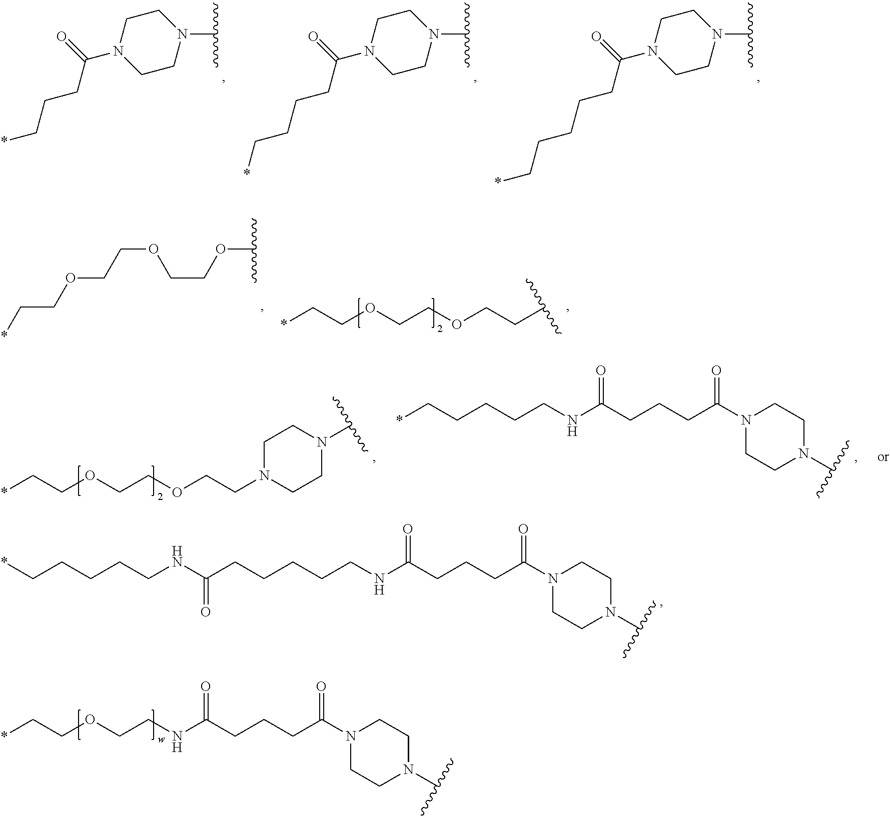

wherein * is a bond to a ZZ group and w is an integer selected from 1-10 (e.g., 2-10, 2-8, 4-8).

In some embodiments, the compound of Formula (XII) is according to one of Formulae (XII-i) through (XII-l) and (XII-s).

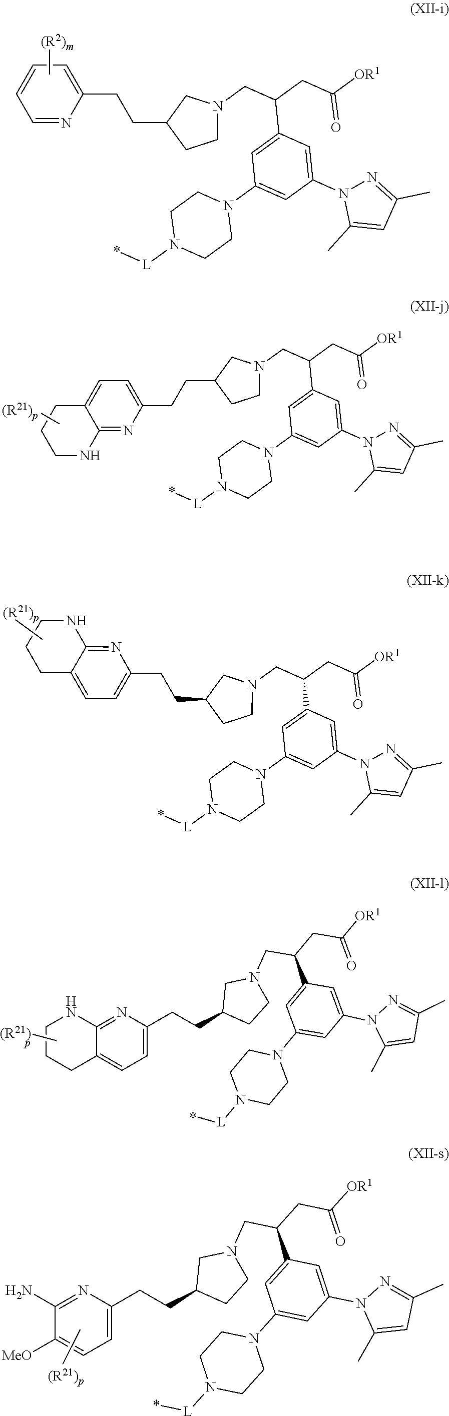

wherein * is a bond to a ZZ group,
p is 0, 1, 2, or 3 (e.g., 0);
each R²¹ is independently selected from group consisting of R and a nitrogen protecting group,
and L is defined according to Formula (XII), or according to any of the preceding embodiments of L.

In one embodiment of Formulae (XII-i) through (XII-1) and (XII-s), R¹ is hydrogen. In another embodiment of Formulae (XII-i) through (XII-1) and (XII-s), R¹ is C₁₋₆alkyl (e.g., methyl or t-butyl).

In some embodiments, the compound of Formula (XII) is according to one of Formulae (XII-m) through (XII-q) and (XII-t):

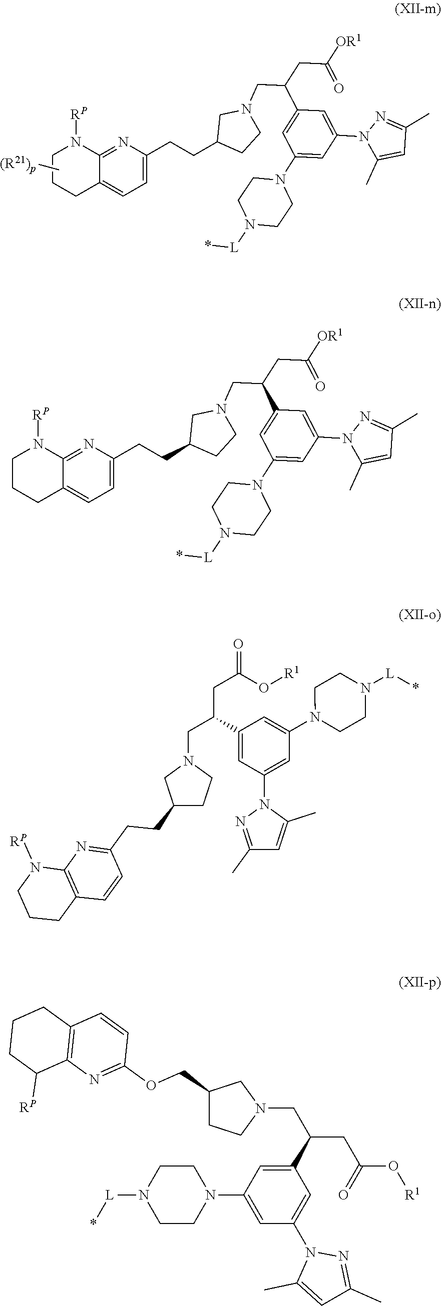

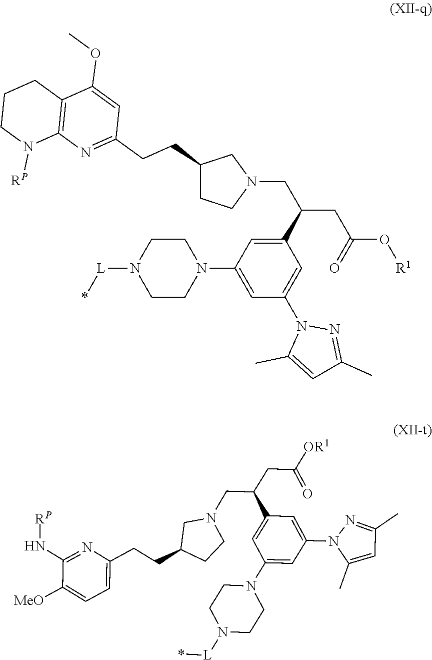

or a salt thereof, wherein * is a bond to a ZZ group,
p is 0, 1 or 2;
each R²¹ is independently selected from group consisting of R;
R^{P} is hydrogen or a nitrogen protecting group (e.g., a nitrogen protecting group),
and L is defined according to Formula (XII), or according to any of the preceding embodiments of L, wherein R and the remaining variables are as defined in Formula (XII).

In one embodiment of Formulae (XII-m) through (XII-q) and (XII-t), R¹ is hydrogen. In another embodiment of Formulae (XII-m) through (XII-q) and (XII-t), R¹ is C₁₋₆alkyl (e.g., methyl or t-butyl).

In one embodiment of the compound of Formula (V) or (XV),
Φ is selected from Formula (XII) and any one of Formula (XII-a) through (XII-q) and (XII-t); ZZ is —CH₂O—, —OCH₂—, —S—S—, —C═N—, —C═N—O—, —C═N—N(R^{N3})—, —N═C—, —O—N═C—, —N(R^{N3})—N═C—, —C(O)N(R^{N3})—, —N(R^{N3})C(O)—, —C(O)O—, —OC(O)—, —OC(O)N(R^{N3})—, —N(R^{N3})C(O)O—, —N(R^{N3})C(O)N(R^{N3})—, —S(O)₂N(R^{N3})—, —N(R^{N3})S(O)₂—, —OP(O)(OH)O—, —OP(S)(OH)O—, —OP(O)(OH)—, —OP(S)(OH)—, —P(O)(OH)O—, or —P(S)(OH)O—. wherein R^{N3} is independently hydrogen or C₁₋₆alkyl;
Δ is selected from,

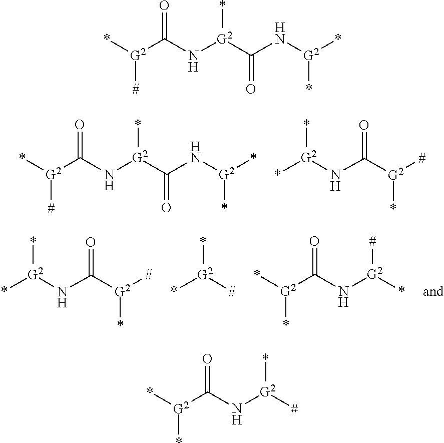

wherein # is the bond to T and each * is a bond to a ZZ group.
   each G² is independently C₁₋₁₀alkyl;
Z⁰ is COOH, NH₂, N₃, hydroxy, —SH, or an activated ester or Z⁰ comprise

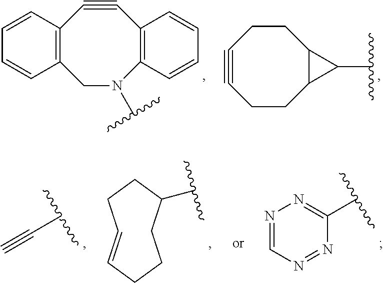

and
R^{T} is

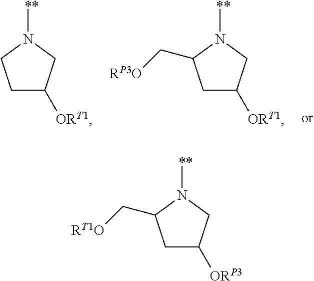

wherein ** represents the bond to T,
   and the remaining variables are as defined in Formula (XV), and any embodiment thereof.

In one embodiment of the compound of Formula (V) or (XV),
Φ is selected from Formula (XII) and any one of Formula (XII-a) through (XII-q) and (XII-t);
ZZ is —C(O)N(R^{N3})—, —N(R^{N3})C(O)—, —C(O)O—, —OC(O)—, wherein R^{N3} is independently hydrogen or C₁₋₆alkyl;
Δ is selected from,

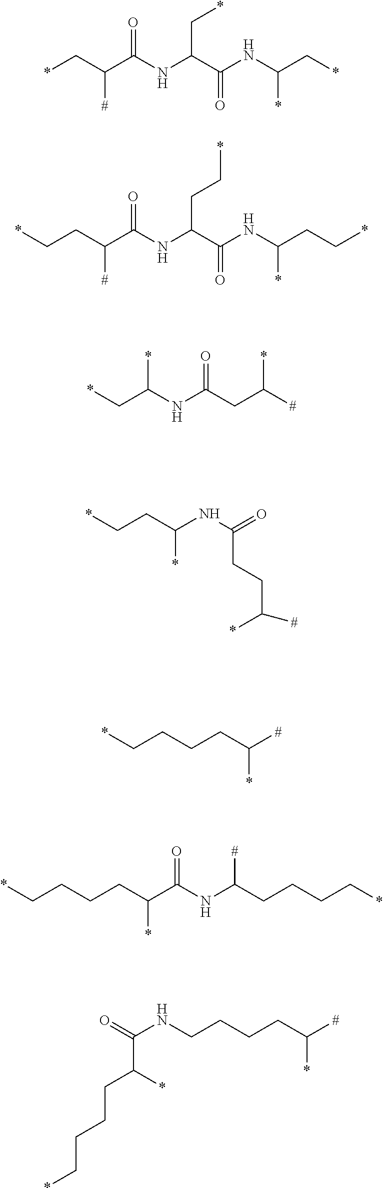

wherein # is the bond to T and each * is a bond to a ZZ group.
   each G² is independently C₁₋₁₀alkyl;
Z⁰ is COOH, NH₂, N₃, hydroxy, —SH, or an activated ester or Z⁰ comprises

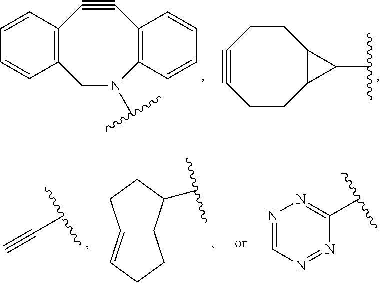

and
R^{T} is

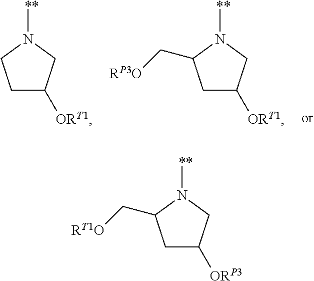

wherein ** represents the bond to T,
   and the remaining variables are as defined in Formula (XV), and any embodiment thereof.

In one embodiment of the compound of Formula (V) or (XV),
Φ is selected from Formula (XII) and any one of Formula (XII-a) through (XII-q) and (XII-t);
ZZ is —CH₂O—, —OCH₂—, —S—S—, —C═N—, —C═N—O—, —C═N—N(R^{N3})—, —N═C—, —O—N═C—, —N(R^{N3})—N═C—, —C(O)N(R^{N3})—, —N(R^{N3})C(O)—, —C(O)O—, —OC(O)—, —OC(O)N(R^{N3})—, —N(R^{N3})C(O)O—, —N(R^{N3})C(O)N(R^{N3})—, —S(O)₂N(R^{N3})—, —N(R^{N3})S(O)₂—, —OP(O)(OH)O—, —OP(S)(OH)O—, —OP(O)(OH)—, —OP(S)(OH)—, —P(O)(OH)O—, or —P(S)(OH)O—. wherein R^{N3} is independently hydrogen or C₁₋₆alkyl; Δ is selected from,

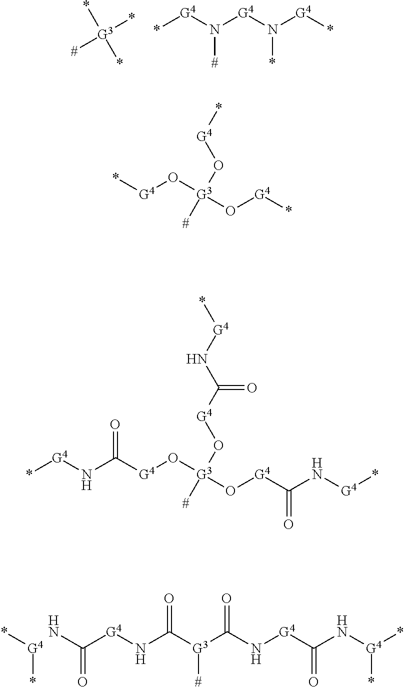

wherein # is the bond to T and each * is a bond to a ZZ group,
G³ is C₁₋₁₀alkyl;
each G⁴ is independently C₁₋₁₀alkyl;
Z⁰ is COOH, NH₂, N₃, hydroxy, —SH, or an activated ester or Z⁰ comprises

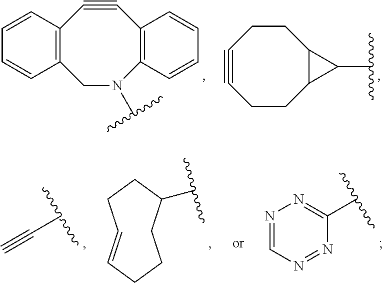

and
R^{T} is

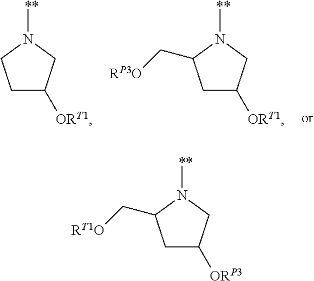

wherein ** represents the bond to T,
   and the remaining variables are as defined in Formula (XV), and any embodiment thereof.

In one embodiment of the compound of Formula (V) or (XV),
Φ is selected from Formula (XII) and any one of Formula (XII-a) through (XII-q) and (XII-t);
ZZ is —C(O)—, —C(O)N(R^{N3})—, —N(R^{N3})C(O)—, —C(O)O—, —OC(O)—, wherein R^{N3} is independently hydrogen or C₁₋₆alkyl;
Δ is selected from,

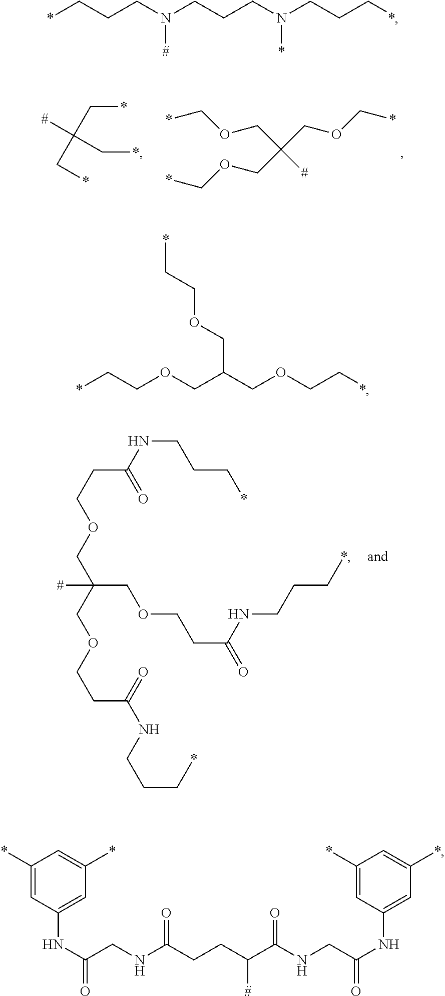

wherein # is the bond to T and each * is a bond to a ZZ group;
Z⁰ is COOH, NH₂, N₃, hydroxy, —SH, or an activated ester or Z⁰ comprises

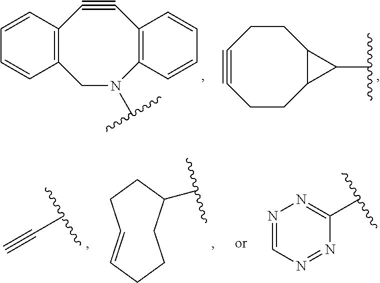

and
R^{T} is

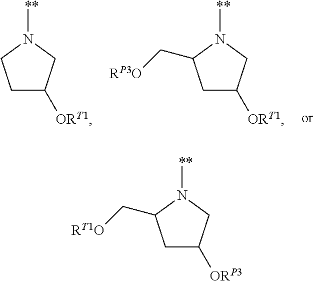

wherein ** represents the bond to T,
   and the remaining variables are as defined in Formula (XV), and any embodiment thereof.

In one embodiment of the compound of Formula (V) or (XV),
Φ is selected from Formula (XII) and any one of Formula (XII-i) through (XII-q) and (XII-t);
ZZ is —OP(O)(OH)O—, —OP(S)(OH)O—, —C(O)N(R^{N3})—, —N(R^{N3})C(O)—, —C(O)O—, —OC(O)—, wherein R^{N3} is independently hydrogen or C₁₋₆alkyl;
Δ is

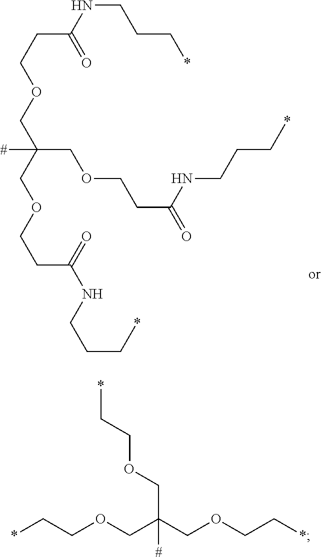

T is **-L⁶-G¹-L⁵-G¹-L⁴-, wherein ** is the bond to Z⁰ or R^{T};
   L⁴ and L⁶ are independently -B¹-A¹- or -A¹-B¹-;
   each L⁵ is a bond or -A¹-B¹-A¹- (e.g., a bond, -B¹-A¹- or -A¹-B¹-; or a bond);
   each G¹ is independently C₁₋₁₀alkyl or C₂₋₁₀alkenyl;
   each A¹ is independently a bond, —O—, —S—, or —N(R^{N1})—, wherein R^{N1} is independently hydrogen or C₁₋₆alkyl; and
   each B¹ is independently a bond, C(O), C(S), S(O)₂, P(O)(OH), or P(S)(OH).
Z⁰ is COOH, NH₂, N₃, hydroxy, —SH, or an activated ester or Z⁰ comprises

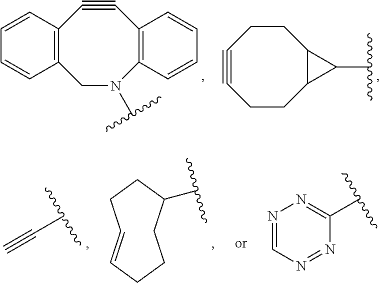

and
R^{T} is

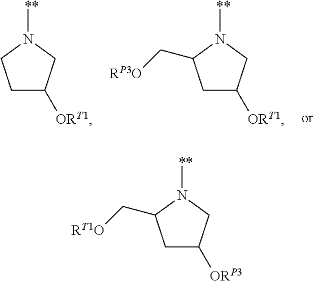

wherein ** represents the bond to T,
   and the remaining variables are as defined in Formula (XV), and any embodiment thereof.

In one embodiment of the compound of Formula (V) or (XV),
Φ is selected from Formula (XII) and any one of Formula (XII-m) through (XII-q) and (XII-t);
ZZ is —OP(O)(OH)O—, —OP(S)(OH)O—, —C(O)N(R^{N3})—, —N(R^{N3})C(O)—, —C(O)O—, —OC(O)—, wherein R^{N3} is independently hydrogen or C₁₋₆alkyl;
Δ is

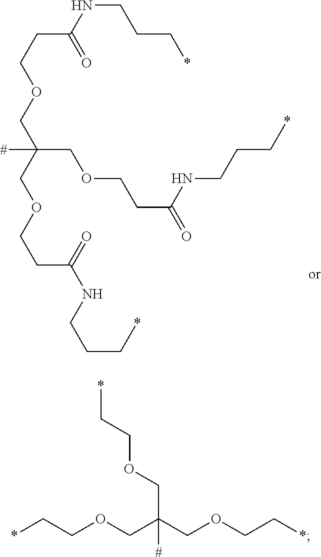

T is **-L⁶-G¹-L⁵-G¹-L⁴-, wherein ** is the bond to Z⁰ or R^{T};
   L⁴ and L⁶ are independently -B¹-A¹- or -A¹-B¹-;
   each L⁵ is a bond or -A¹-B¹-A¹- (e.g., a bond, -B¹-A¹- or -A¹-B¹-; or a bond);
   each G¹ is independently C₁₋₁₀alkyl or C₂₋₁₀alkenyl;
   each A¹ is independently a bond, —O—, —S—, or —N(R^{N1})—, wherein R^{N1} is independently hydrogen or C₁₋₆alkyl; and
   each B¹ is independently a bond, C(O), C(S), S(O)₂, P(O)(OH), or P(S)(OH).
Z⁰ is COOH, NH₂, N₃, hydroxy, —SH, or an activated ester or Z⁰ comprises

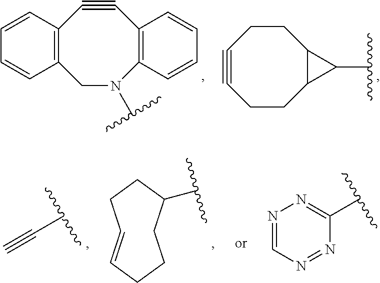

and
R^{T} is

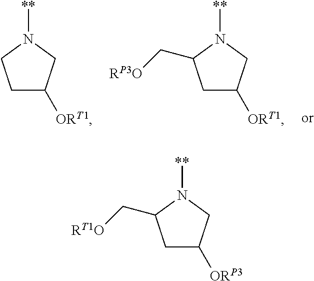

wherein ** represents the bond to T,
   and the remaining variables are as defined in Formula (XV), and any embodiment thereof.

In one embodiment of the compound of Formula (V) or (XV),
Φ is selected from Formula (XII) and any one of Formula (XII-m) through (XII-q) and (XII-t);
ZZ is —C(O)N(H)— or —N(H)C(O)—;
   Δ is
   T is **-L⁶-G¹-L⁵-G¹-L⁴-, wherein ** is the bond to Z⁰ or R^{T};
L⁴ is -A¹-B¹-;
L⁶ is -B¹-;
L⁵ is a bond, -B¹-A¹-, or -A¹-B¹-;
each G¹ is independently C₁₋₁₀alkyl;
each A¹ is independently a bond, —O— or —N(H);
each B¹ is C(O);
   Z⁰ is COOH, NH₂, N₃, hydroxy, —SH, or an activated ester or Z⁰ comprises

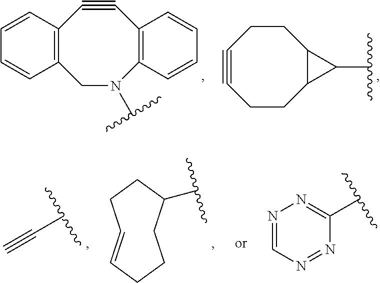

and

### R^{T} is

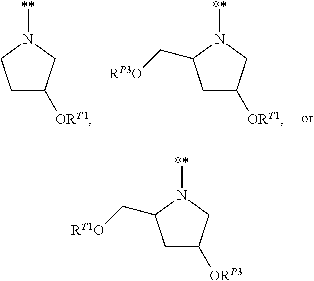

wherein * * represents the bond to T, and the remaining variables are as defined in Formula (XV), and any embodiment thereof.

In one embodiment of the compound of Formula (XV) is according to the structure,

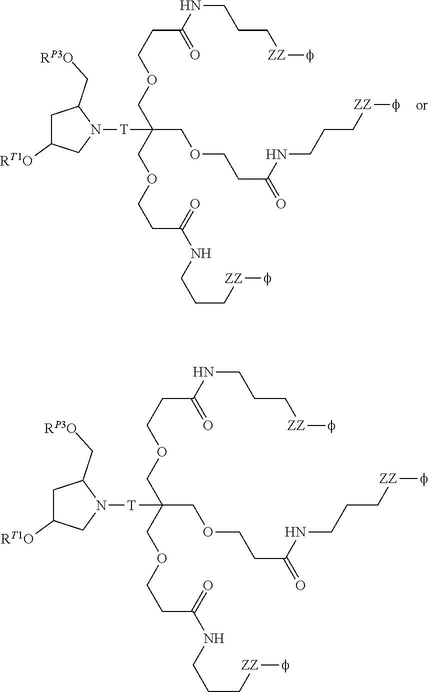

wherein R^{P3} is hydrogen or a hydroxyl protecting group (e.g., 4,4′-dimethoxytrityl (DMTr))
Φ is selected from Formula (XII) and any one of Formula (XII-m) through (XII-q) and (XII-t); ZZ is —C(O)N(H)— or —N(H)C(O)—; and
T is **-L⁶-G¹-L⁵-G¹-L⁴-, wherein ** is the bond to Z⁰ or R^{T};
   L⁴ is -A¹-B¹-;
   L⁶ is -B¹-;
   L⁵ is a bond, -B¹-A¹-, or -A¹-B¹-;
   each G¹ is independently C₁₋₁₀alkyl;
each A¹ is independently a bond, —O— or —N(H);
each B¹ is C(O);
   and the remaining variables are as defined in Formula (XV), and any embodiment thereof.

In one embodiment of the compound of Formula (V) is according to the structure,

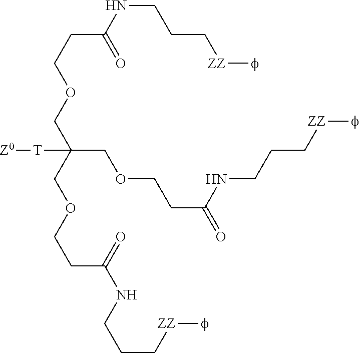

Φ is selected from Formula (XII) and any one of Formula (XII-m) through (XII-q) and (XII-t); ZZ is —C(O)N(H)— or —N(H)C(O)—; and
T is **-L⁶-G¹-L⁵-G¹-L⁴-, wherein ** is the bond to Z⁰ or R^{T};
   L⁴ is -A¹-B¹-;
   L⁶ is -B¹-;
   L⁵ is a bond, -B¹-A¹-, or -A¹-B¹-;
   each G¹ is independently C₁₋₁₀alkyl;
   each A¹ is independently a bond, —O— or —N(H);
   each B¹ is C(O); and
Z is COOH, NH₂, N₃, hydroxy, —SH, or an activated ester or Z⁰ comprises

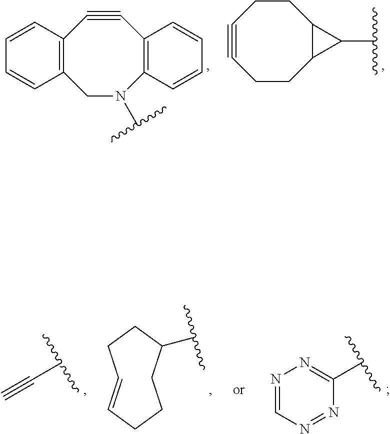

and the remaining variables are as defined in Formula (XV), and any embodiment thereof.

In one embodiment of the compound of Formula (V) or (XV),
Φ is selected from Formula (XII) and any one of Formula (XII-m) through (XII-q) and (XII-t);
ZZ is —OP(O)(OH)O—, —OP(S)(OH)O—, —C(O)N(R^{N3})—, —N(R^{N3})C(O)—, —C(O)O—, —OC(O)—, wherein R^{N3} is independently hydrogen or C₁₋₆alkyl;
Δ is r

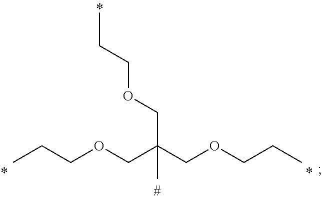 [SMILES: CCCOCC(C)(COCCC)COCCC]

T is ***-L⁶-[G⁵-O]_{q5}-G⁵-L⁴-, wherein
   q5 is an integer selected from 1 to 20;
   L⁴ and L⁶ are independently -A¹-B¹-A¹-, wherein
      each A¹ is independently a bond, —O—, —S—, or —N(R^{N1})—, wherein R^{N1} is hydrogen or C₁₋₆alkyl; and
      each B¹ is independently a bond, C(O), C(S), S(O)₂, P(O)(OH), or P(S)(OH);
      each G⁵ is independently C₁₋₁₀alkyl;
Z is COOH, NH₂, N₃, hydroxy, —SH, or an activated ester or Z⁰ comprises

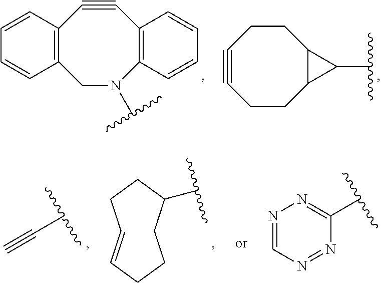

and
R^{T} is

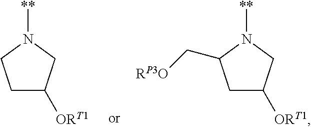

wherein ** represents the bond to T,
   and the remaining variables are as defined in Formula (XV), and any embodiment thereof.

In one embodiment of the compound of formula (XV), is of the structure,

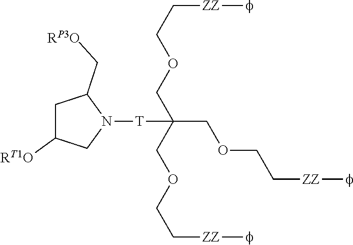

wherein
Φ is selected from Formula (XII) and any one of Formula (XII-m) through (XII-q) and (XII-t), wherein L is

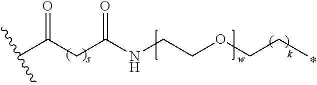

wherein * is the bond to ZZ; s and k are independently is an integer from 1 to 20 (e.g., an integer from 1 to 16, an integer from 1 to 10, an integer from 3 to 10, an integer from 3 to 7 or an integer from 4 to 6); and w is an integer from 1 to 10 (e.g., an integer from 1 to 16, an integer from 1 to 10, an integer from 3 to 10, an integer from 3 to 7 or an integer from 4 to 6); ;
ZZ is —C(O)N(H)—, —N(H)C(O)—;
T is ***-L⁶-[G⁵-O]_{q5}-G⁵-L⁴-, wherein
   q5 is an integer selected from 1 to 20;
   L⁴ and L⁶ are independently -A¹-B¹-A¹-, wherein
      each A¹ is independently a bond, —O—, —S—, or —N(R^{N1})—, wherein R^{N1} is hydrogen or C₁₋₆alkyl; and each B¹ is independently a bond, C(O), C(S), S(O)₂, P(O)(OH), or P(S)(OH);
      each G⁵ is independently C₁₋₁₀alkyl;
         and the remaining variables are as defined in Formula (XV), and any embodiment thereof.

In one embodiment of the compound of formula (XV), is of the structure,

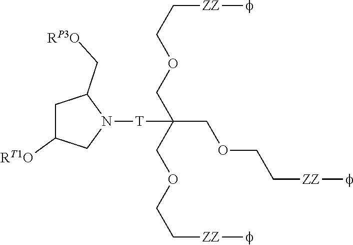

wherein
Φ is selected from Formula (XII) and any one of Formula (XII-m) through (XII-q) and (XII-t), wherein L is

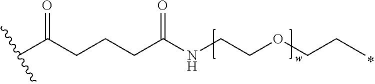

wherein * is the bond to ZZ and w is an integer from 1 to 20 (e.g., an integer from 1 to 16, an integer from 1 to 10, an integer from 3 to 10, an integer from 3 to 7 or an integer from 4 to 6;
ZZ is —C(O)N(H)—, —N(H)C(O)—;
T is **—C(O)—[CH₂CH₂—O]_{q5}—C₂₋₁₀alkyl-C(O)N(H)—, wherein ** is the bond to the pyrrolidine ring and q5 is an integer selected from 1 to 20 (e.g., 1 to 10, or 2 to 10; or 2-8; or 1; or 2; or 3; or 4); and the remaining variables are as defined in Formula (XV), and any embodiment thereof.

### R^{T1} Embodiments, Formula (XV)

In embodiments of any embodiments of R^{L} of Formula (XV), Formula (XV-a) through (XV-k),
R^{T1} is -L^{L}-oligonucleotide, wherein L^{L} is a divalent linker that connects to the 3′-end of the oligonucleotide, the 5′-end of the oligonucleotide, or an internal 2′- or 3′ position on a internal nucleotide (i.e., a nucleotide that is not the 5′-terminal or 3′-terminal nucleoside).

In some embodiments, R^{T1} is -L^{L}-oligonucleotide, wherein L^{L} is a divalent linker that connects to the 3′-end of the oligonucleotide, such as one of:
directly to the 3′-carbon of the 3′-terminal nucleoside;
directly to the 3′-O of the 3′-terminal nucleoside;
directly to the 4′-carbon of the 3′-terminal nucleoside;
directly to the 2′-carbon of the 3′-terminal nucleoside; or
directly to the 2′-O of the 3′-terminal nucleoside.

In some embodiments, R^{T1} is -L^{L}-oligonucleotide, wherein L^{L} is a divalent linker that connects to the 5′-end of the oligonucleotide, such as one of:
directly to the 5′-carbon of the 5′-terminal nucleoside;
directly to the 5′-O of the 5′-terminal nucleoside;
directly to the 4′-carbon of the 5′-terminal nucleoside;
directly to the 2′-carbon of the 5′-terminal nucleoside; or
directly to the 2′-O of the 5′-terminal nucleoside.

In some embodiments, R^{T1} is -L^{L}-oligonucleotide, wherein L^{L} is a divalent linker that connects to an internal 2′- or 3′ position on an internal nucleotide.

In some embodiments, R^{T1} is -L^{L}-oligonucleotide, wherein L^{L} is a divalent linker that connects to an internal 2′-position on a internal nucleotide.

In another embodiment of any of the preceding embodiments of R^{T1}, when L^{L} connects to a carbon atom on a nucleoside, then L^{L} is -B³-A³-, wherein
B³ is —P(O)(OH)—, —P(S)(OH)—, or —P(S)(SH)—; and
A³ is —O—, —S—, or —N(H)—.

In another embodiment, when L^{L} connects to a carbon atom on a nucleoside, then L^{L} is -B³-A³-, wherein
B³ is —P(O)(OH)— or —P(S)(OH)—; and
A³ is —O—.

In another embodiment, when L^{L} connects to a oxygen atom on a nucleoside, then L^{L} is —P(O)(OH)—, —P(S)(OH)—, or —P(S)(SH).

In another embodiment, when L^{L} connects to a oxygen atom on a nucleoside, then L^{L} is —P(O)(OH)— or —P(S)(OH)—.

In another embodiment, when L^{L} connects to a oxygen atom on a nucleoside, then L^{L} is —P(O)(OH)—.

In another embodiment, when L^{L} connects to a oxygen atom on a nucleoside, then L^{L} is —P(S)(OH)—.

In another embodiment, when L^{L} connects to a oxygen atom on a nucleoside, then
L^{L} is -B³-A³-L^{L1}-A³-B³-, wherein
   B³ is a bond, —C(O)—, C(S)—, C(NH), S(O), S(O)₂, —P(O)(OH)—, —P(S)(OH)—, or —P(S)(SH);
   A³ is a bond, —O—, —S—, or —N(H)—; and
   L^{L1} is C₁₋₁₀alkyl, C₂₋₁₀alkenyl, C₂₋₁₀alkynyl, C₃₋₁₀cycloalkyl, 3-10 membered heterocyclyl, aryl, or heteroaryl.

In another embodiment, when L^{L} connects to a oxygen atom on a nucleoside, then L^{L} is -B³-L^{L1}-B³—, wherein B³ is —C(O)—; and L^{L1} is C₁₋₁₀alkyl.

In certain embodiments, where R^{T} is R^{T1}, wherein R^{T1} is -L^{L}-oligonucleotide, when L^{L} connects to a oxygen atom on a nucleoside, and L L⁵ is a bond, the nucleoside is of Formula (XV-f),

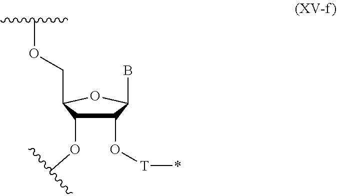

wherein B is an optionally modified nucleobase (e.g., adenine, cytosine, uracil, guanine, 5-methylcytosine, or -5-methyluracil); L¹ is according any of the preceding embodiments; and * represent the bond to ZZ.

In some embodiments of Formula (XV-f), -T- is -L¹-G-L³-*, wherein * is the bond to ZZ.

In some embodiments, -T- is —C₂₋₃₀alkyl-*, wherein the alkyl is optionally substituted with one or two R groups, and * is the bond to ZZ. In some embodiments, -T- is —C₂₋₃₀alkyl-*, wherein the alkyl is optionally substituted with one or two groups selected from the group consisting of halogen, hydroxy, C₁₋₆alkoxy, amino, C₁₋₆alkylamino, di(C₁₋₆alkylamino), cyano, carboxy, and * is the bond to ZZ. In some embodiments, -T- is —C₂₋₃₀alkyl-*, wherein the alkyl is optionally substituted with one group selected from the group consisting of halogen, hydroxy, C₁₋₆alkoxy, amino, C₁₋₆alkylamino, di(C₁₋₆ alkylamino), cyano, carboxy, and * is the bond to ZZ. In some embodiments, -T- is —C₂₋₃₀alkyl-*, wherein the alkyl is optionally substituted with one group selected from the group consisting of hydroxy, amino, and carboxy, and * is the bond to ZZ. In some embodiments, -T- is —C₂₋₃₀alkyl-*, wherein the alkyl is optionally substituted with hydroxy, and * is the bond to ZZ.

In some embodiments, -T- is —C₂₋₃₀alkyl-*, wherein * is the bond to ZZ. In some embodiments, -T- is —C₂₋₁₀alkyl-*, wherein * is the bond to ZZ. In some embodiments, -T- is —C₄₋₁₂alkyl-*, wherein * is the bond to ZZ. In some embodiments, -T- is —C₄₋₁₀alkyl-*, wherein * is the bond to ZZ. In some embodiments, -T- is —C₅₋₁₀alkyl-*, wherein * is the bond to ZZ. In some embodiments, -T- is —C₆alkyl-*, wherein * is the bond to ZZ. In some embodiments, -T- is —C₈alkyl-*, wherein * is the bond to ZZ. In some embodiments, -T- is —C₁₀alkyl-*, wherein * is the bond to ZZ.

In some embodiments of Formula (XV-f), -T- is -L¹-[G-L²]_{q}-G-L³-*, wherein
* is the bond to ZZ;
q is 0, 1, 2, 3, 4, or 5;
L¹ is a bond or -B-A-;
each L² is independently -A-B-A-;
L³ is a bond or -A-B-A-;
each A is independently a bond, —O—, —S—, or —N(R^{N})—;
each B is independently a bond, CH₂, C(O), C(S), C(NR^{N}), S(O)₂, P(O)(OH), or P(S)(OH);
each R^{N} is independently hydrogen or C₁₋₆alkyl; and
each G is independently C₁₋₁₀alkyl, C₂₋₁₀alkenyl, C₂₋₁₀alkynyl, each of which is optionally substituted with 1, 2, 3, or 4 R groups.

In some embodiments, -T- is -L¹-[G-L²]_{q}-G-*, wherein * is the bond to ZZ, q is 0, 1, 2, or 3; L¹ is a bond or -B-A-; and
(c) each L² is independently a bond, C(O)O, OC(O), C(O)(NR^{N}), N(R^{N})C(O), SO₂N(R^{N}), N(R^{N})SO₂, OP(O)(OH), OP(S)(OH), P(O)(OH)O, P(S)(OH)O, OP(O)(OH)O, or OP(S)(OH)O, wherein each R^{N} is independently hydrogen or C₁₋₆alkyl; each G is independently C₁₋₁₀alkyl, C₂₋₁₀alkenyl, C₂₋₁₀alkynyl, each of which is optionally substituted with 1 or 2 R groups;
or
(d) each L² is independently a bond, C(O)O, OC(O), C(O)(NR^{N}), N(R^{N})C(O), OP(O)(OH)O, or OP(S)(OH)O, wherein each R^{N} is independently hydrogen or C₁₋₆alkyl; and each G is independently C₁₋₁₀alkyl or C₂₋₁₀alkenyl, each of which is optionally substituted with 1 or 2 R groups.

In some embodiments, -T- is -[G-L²]_{q}-G-*, wherein * is the bond to ZZ, q is 0, 1, 2, or 3 (e.g., q is 0, 1, or 2; or 0 or 1; or 0; or 1; or 2); each G is independently C₁₋₁₀alkyl, each of which is optionally substituted with 1 or 2 R groups. and
(e) each L² is independently C(O)O or OC(O);
(f) each L² is independently C(O)(NR^{N}) or N(R^{N})C(O), wherein each R^{N} is independently hydrogen or C₁₋₆alkyl
(g) each L² is independently OP(O)(OH)O, or OP(S)(OH)O (e.g., each is OP(O)(OH)O);
or
(h) each L² is a bond.

In some embodiments, the compound of Formula (XV) is according to one of Formulae (XV-g) through (XV-q):

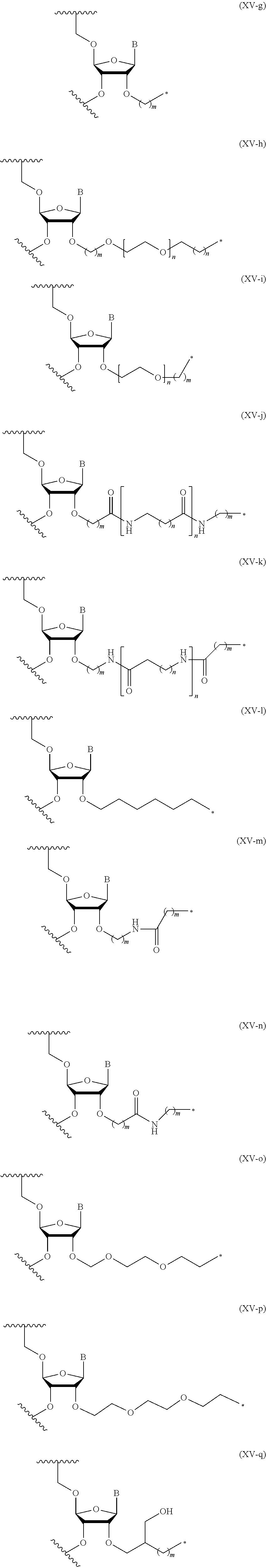

wherein
B is an optionally modified nucleobase (e.g., adenine, cytosine, uracil, guanine, 5-methylcytosine, or -5-methyluracil);
each n is independently 0 or an integer selected from 1-10; (e.g., 1-5, or 1-3, or 3, or 2, or 1);
each m is independently integer selected from 1-20 (e.g., 2-12, or 2-10; or 2-6; or 2; or 3; or 4; or 5; or 6).

In some embodiments, the compound of Formula (XV) is according to one of Formulae (XV-r) through (XV-w):

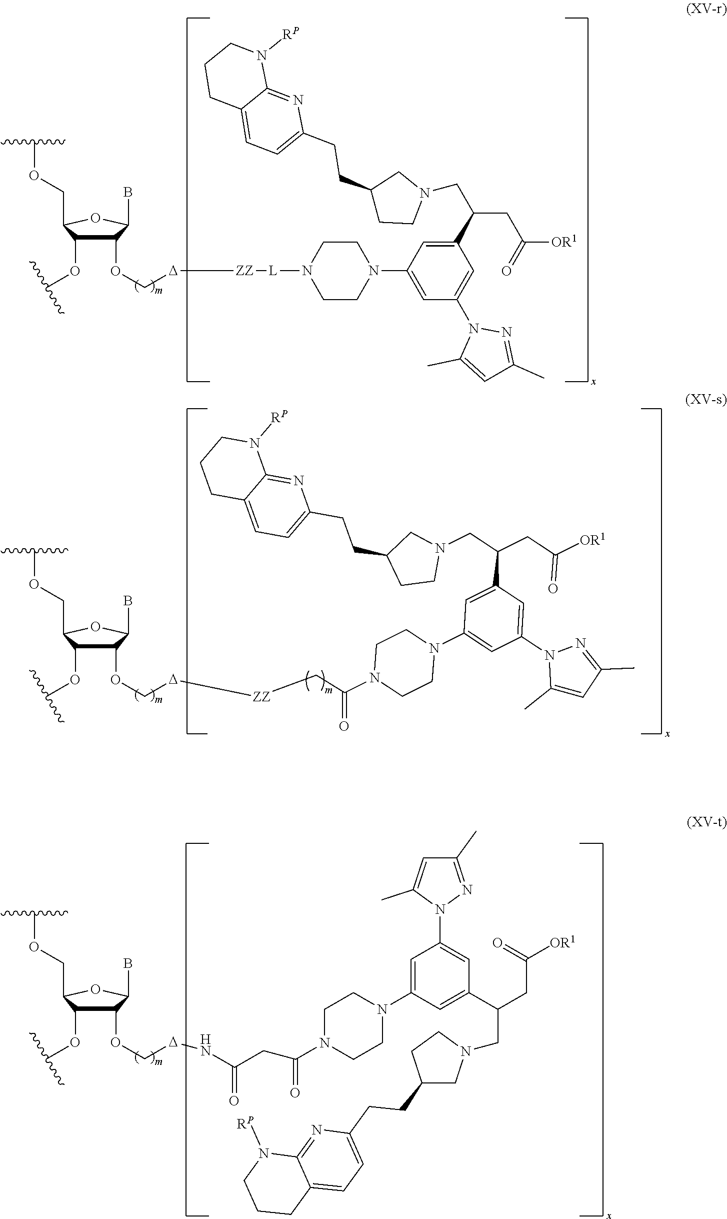

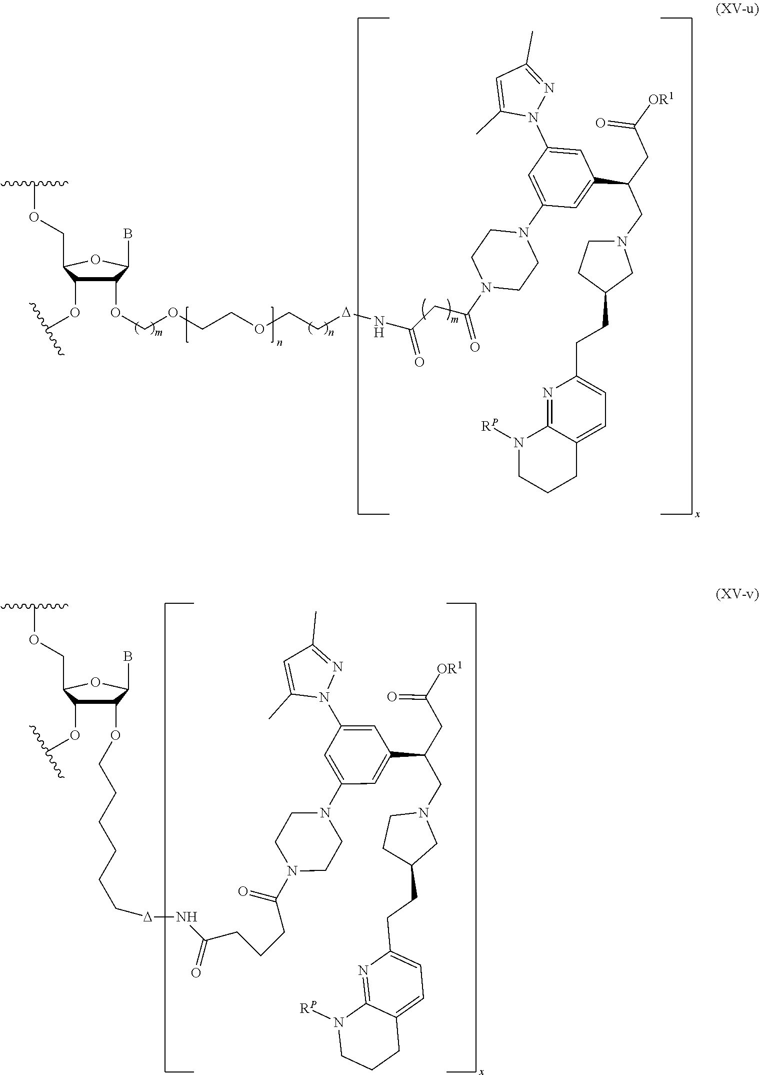

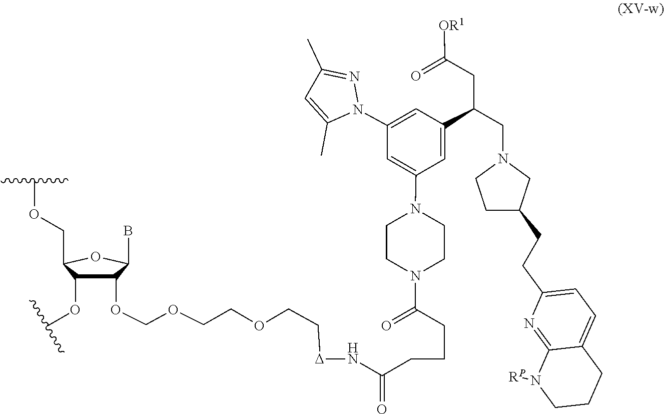

or a salt thereof, wherein
L and ZZ are as defined in Formula (XV) or in any embodiment preceding or below;
B is an optionally modified nucleobase (e.g., adenine, cytosine, uracil, guanine, 5-methylcytosine, or -5-methyluracil);
each m is independently integer selected from 1-20 (e.g., 2-12, or 2-10; or 2-6; or 2; or 3; or 4; or 5; or 6);
R^{P} is hydrogen or a nitrogen protecting group (e.g., a nitrogen protecting group); and
R¹ is hydrogen or C₁₋₆alkyl (e.g., methyl ort-butyl).

In one embodiment of Formulae (XV-r) through (XV-w), R¹ is hydrogen. In another embodiment of Formulae (XV-r) through (XV-w), R¹ is C₁₋₆alkyl (e.g., methyl or t-butyl). In another embodiment of Formulae (XV-r) through (XV-w), R¹ is hydrogen and R^{P} is hydrogen. In another embodiment of Formulae (XV-r) through (XV-w), R¹ is hydrogen and a nitrogen protecting group. In another embodiment of Formulae (XV-r) through (XV-w), R¹ is C₁₋₆alkyl (e.g., methyl or t-butyl) and R^{P} is hydrogen. In another embodiment of Formulae (XV-r) through (XV-w), R¹ is C₁₋₆alkyl (e.g., methyl or t-butyl) and a nitrogen protecting group.

In certain embodiments, where R^{T} is R^{T1}, wherein R^{T1} is -L^{L}-oligonucleotide, when L^{L} connects to a oxygen atom or nitrogen atom in an internucleotide linkage, the internucleotide linkage can be of the formula, including the 3′ and 5′ oxygen atoms of the preceding and following nucleosides, respectively,

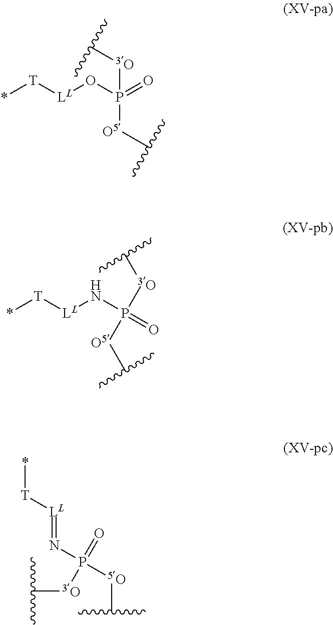

wherein L′ can be, for example a bond, —S(O)₂— or, in for Formula (XV-i), a 5-8 membered heterocyclyl ring optionally substituted with 1 or 2 R groups, as defined herein; and * represent the bond to Δ.

For example, the preceding includes,

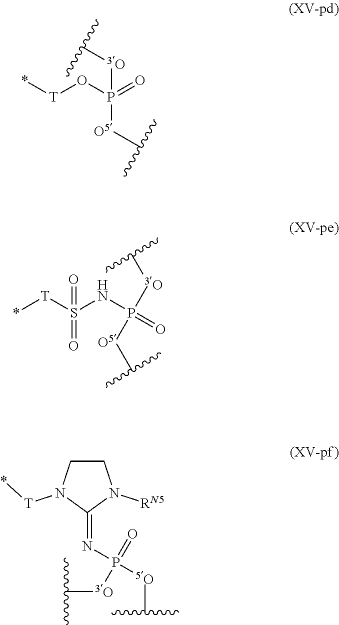

wherein * represent the bond to Δ; and R^{N5} is hydrogen or C₁₋₁₀ alkyl.

For example, the preceding includes,

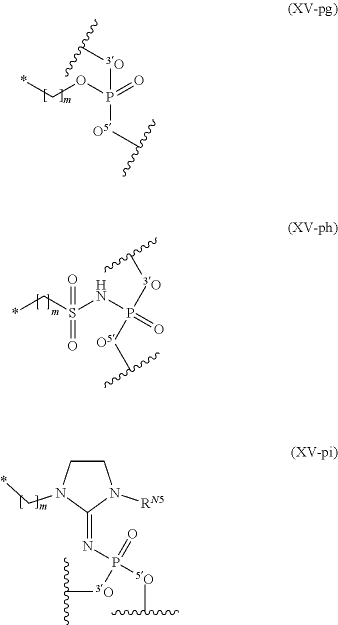

wherein * represent the bond to Δ; m is an integer selected from 1-20 (e.g., 1-10, or 2-20, or 2-10, or 4-10, or 4-8; or 6-12; or 5; or 6; or 7; or 8; or 9; or 10), and R^{N5} is hydrogen or C₁₋₁₀ alkyl.

In another embodiment, the compound of Formula (XV-pd) is

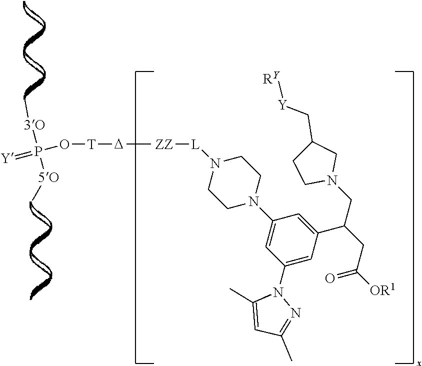

wherein Y′ is O or S, and R^{Y}, Y, R¹, L, T, Δ, and ZZ are as defined for Formula (XV). In one embodiment, R¹ is hydrogen. In another embodiment, R¹ is C₁₋₆alkyl (e.g., methyl or t-butyl).

In another embodiment, the compound is

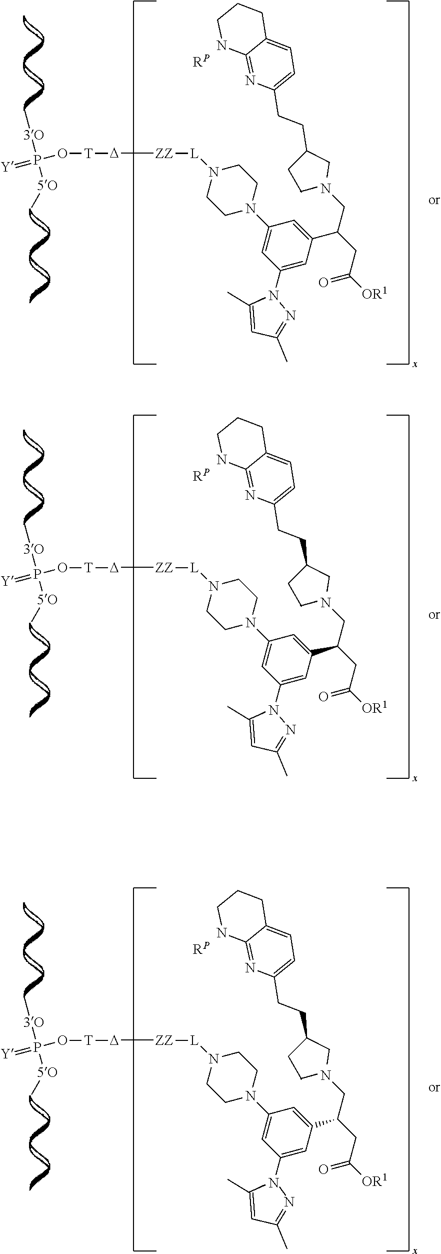

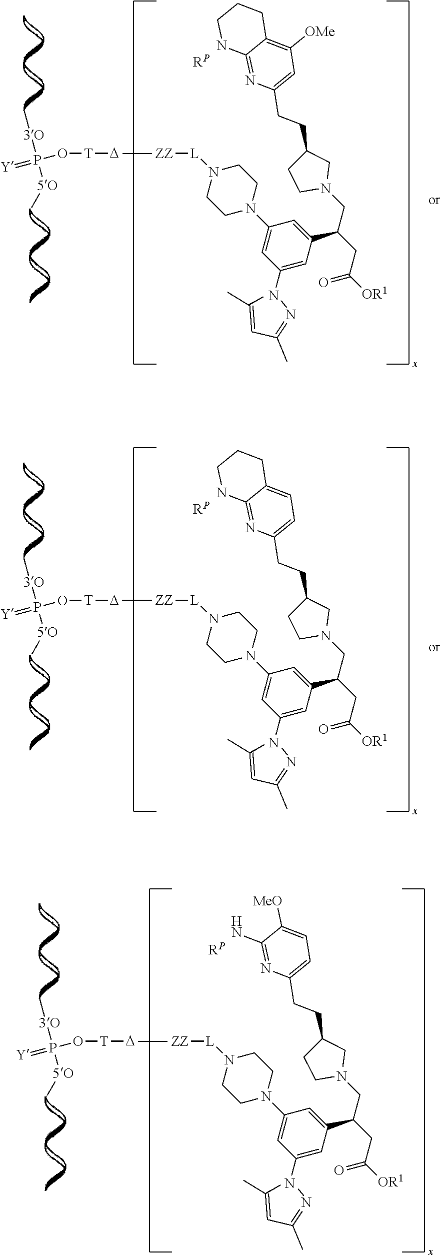

wherein Y′ is O or S, and R¹, T, Δ, R^{P}, L, and ZZ are as defined for Formula (XV).

In another embodiment, the compound is

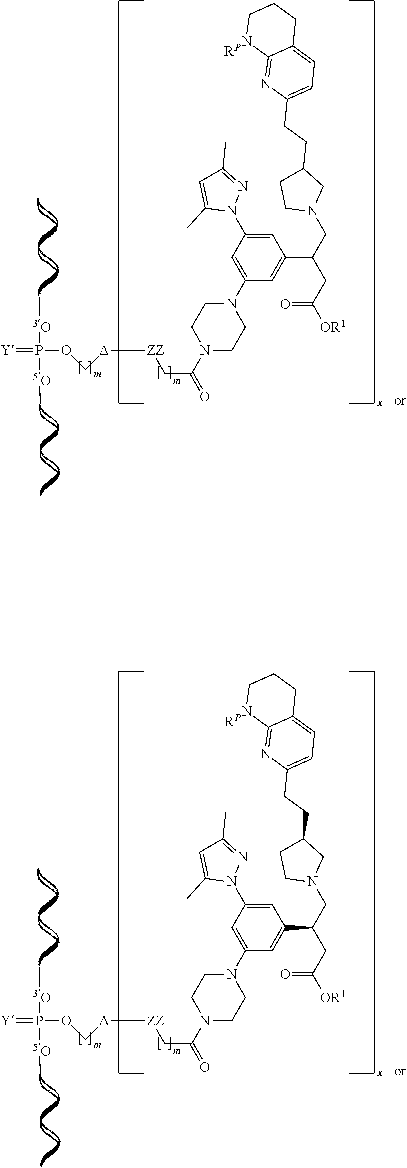

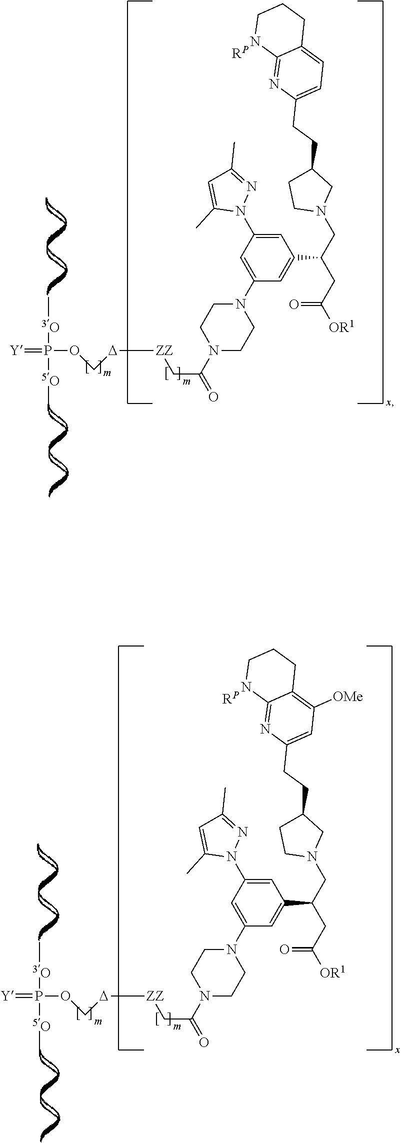

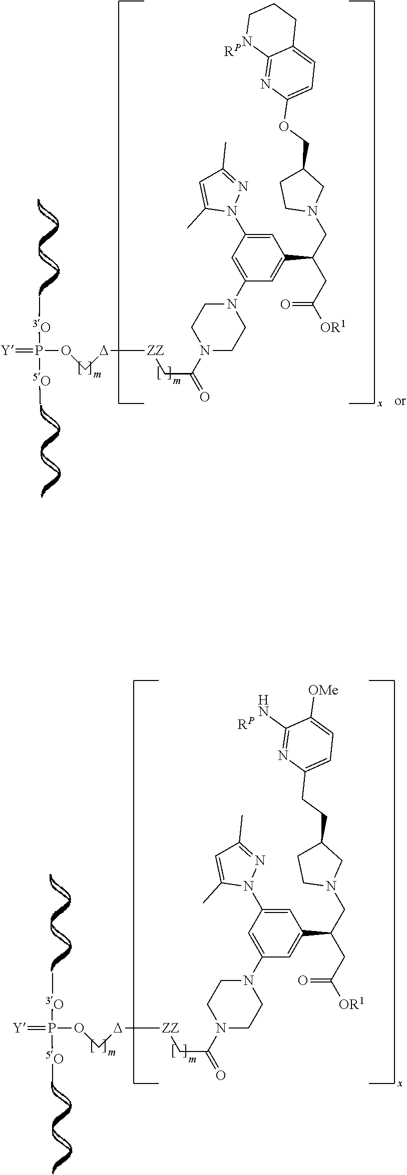

wherein each m is an integer selected from 1-20 (e.g., 2-20, 2-10, 1-10, 2-16, 4-16, 4-8, or 6-12), Y′ is O or S, and R¹, Δ, R^{P} and ZZ are as defined for Formula (XV).

In another embodiment, the compound of Formula (XV-pe) is

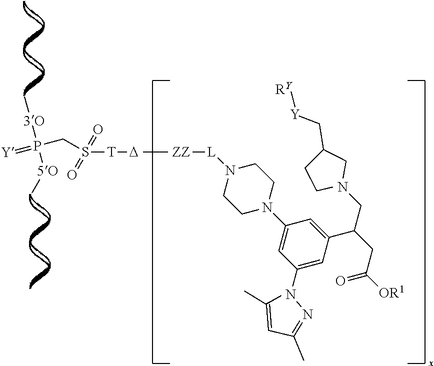

wherein Y′ is O or S, and R^{Y}, Y, R¹, L, T, Δ, and ZZ are as defined for Formula (XV). In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound is

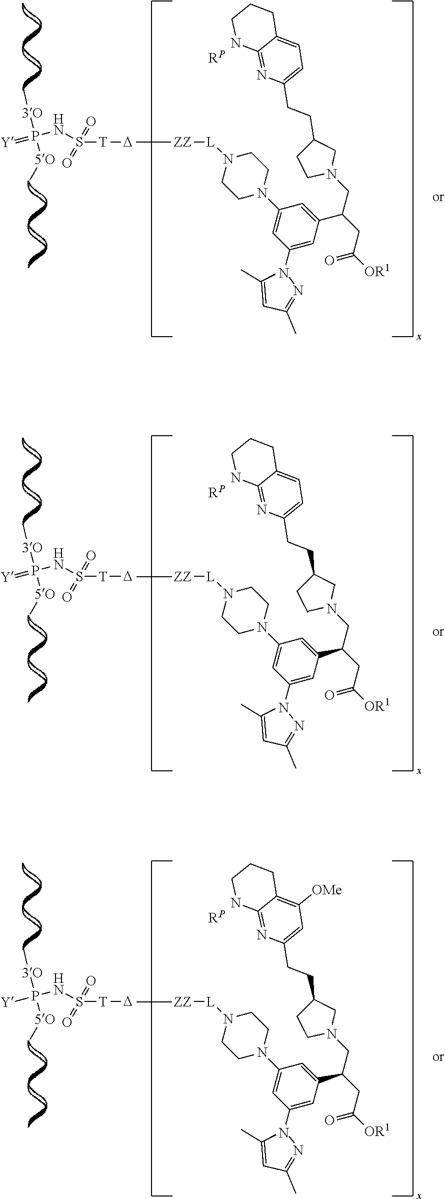

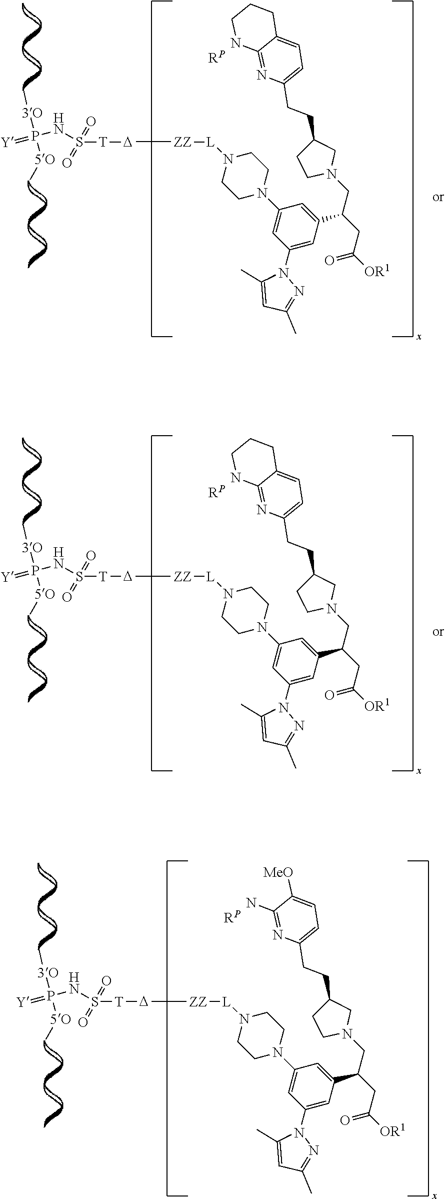

wherein Y′ is O or S, and R¹, L, T, Δ, R^{P}, and ZZ are as defined for Formula (XV). In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound is

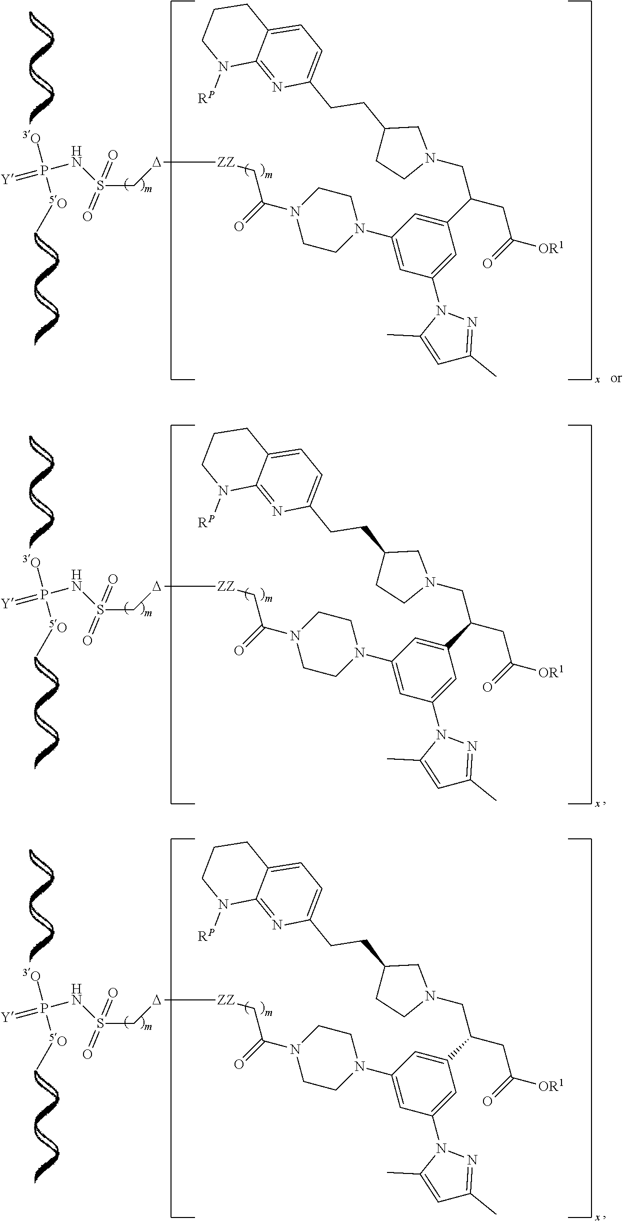

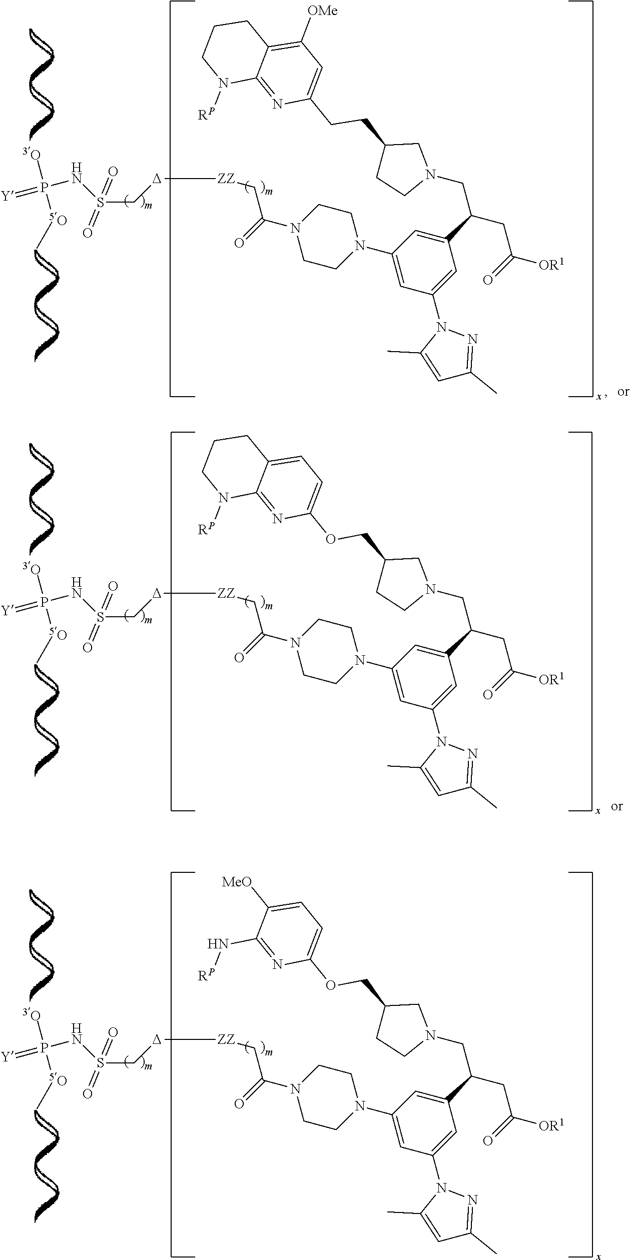

wherein each m is an integer selected from 1-20 (e.g., 2-20, 2-10, 1-10, 2-16, 4-16, 4-8, or 6-12), Y′ is O or S, and R¹, Δ, R^{P}, and ZZ are as defined for Formula (XV).

In one embodiment, Y′ is O. In another embodiment, Y′ is S.

In one embodiment, R¹ is hydrogen. In another embodiment, R¹ is C₁₋₆alkyl (e.g., methyl or t-butyl).

In one embodiment, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment, Y′ is O and R¹ is hydrogen. In one embodiment, Y′ is O and R¹ is C₁₋₆ alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, when L^{L} connects to a oxygen atom on a nucleoside, then L^{L} is—a bond.

For Formula (XV), when R^{T1} is -L^{L}-oligonucleotide and is conjugated at the 5′-end of the oligonucleotide, in one embodiment, R^{T1} can be represented as Formula (XV-5′)

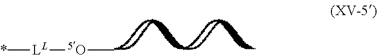

wherein L^{L} is —P(Y)(OH)—, wherein Y is O or S (e.g., S); and * represents the bond to remainder of the compound of Formula (XV).

In another embodiment of Formula (XV), when R^{T1} is -L^{L}-oligonucleotide and is conjugated at the 5′-end of the oligonucleotide, in one embodiment, R^{T1} can be represented as Formula (XV-5′o) or Formula (XV-5's),

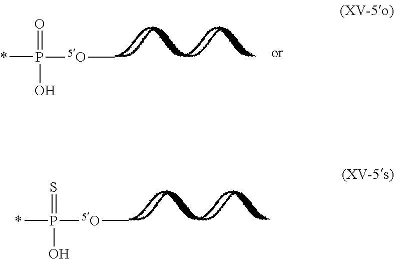

wherein * represents the bond to remainder of the compound of Formula (XV).

In another embodiment, a compound of Formula (XV) can be represented by,

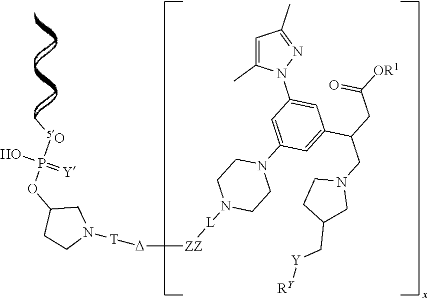

wherein Y′ is O or S, x is an integer selected from 2 to 8, and Δ, R^{Y}, Y, R¹, L, T, and ZZ are as defined for Formula (XV) or any embodiment thereof.

For Formula (XV), when R^{T1} is -L^{L}-oligonucleotide and is conjugated at the 3′-end of the oligonucleotide, in one embodiment, R^{T1} can be represented as Formula (XV-3′)

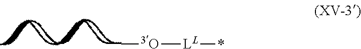

wherein L^{L} is —P(Y)(OH)—, wherein Y is O or S (e.g., S); and * represents the bond to remainder of the compound of Formula (XV).

In another embodiment of Formula (XV), when R^{T1} is -L^{L}-oligonucleotide and is conjugated at the 3′-end of the oligonucleotide, in one embodiment, R^{T1} can be represented as Formula (XV-3′o) or Formula (XV-3's),

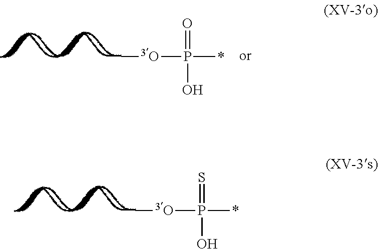

wherein * represents the bond to remainder of the compound of Formula (XV).

In another embodiment, a compound of Formula (XV) can be represented by,

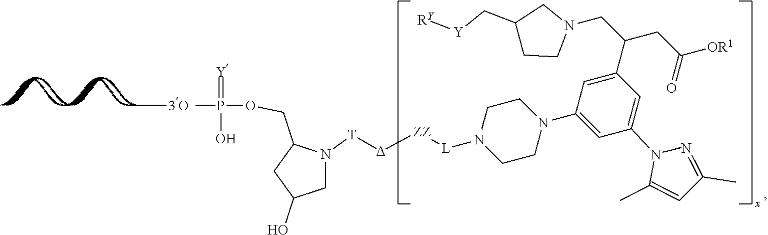

wherein Y′ is O or S, x is an integer selected from 2 to 8, and Δ, R^{Y}, Y, R¹, L, T, and ZZ are as defined for Formula (XV) or any embodiment thereof. In one embodiment, R¹ is hydrogen. In another embodiment, R¹ is C₁₋₆alkyl (e.g., methyl or t-butyl).

In another embodiment, the compound of Formula (XV) is

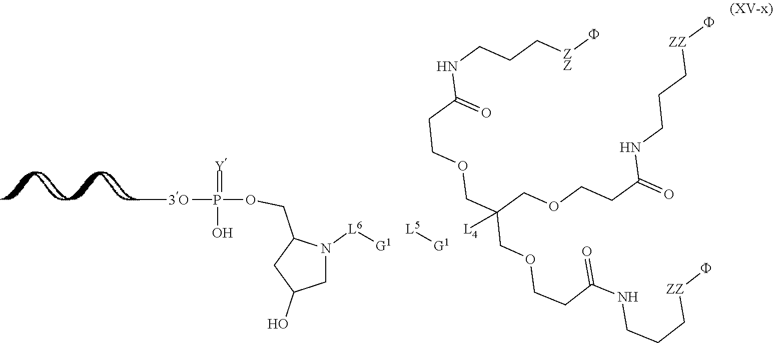

wherein Y′ is O or S;
each ZZ is N(H)C(O) or C(O)N(H);
each Φ is

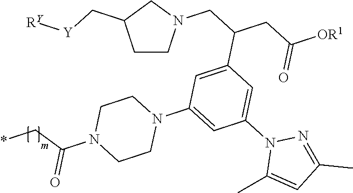

wherein m is an integer selected from 1-10;
   L⁴ and L⁶ are independently -B¹-A¹- or -A¹-B¹-;
   each L⁵ is a bond or -A¹-B¹-A¹- (e.g., a bond, -B¹-A¹- or -A¹-B¹-; or a bond);
   each G¹ is independently C₁₋₁₀alkyl or C₂₋₁₀alkenyl;
   each A¹ is independently a bond, —O—, —S—, or —N(R^{N1})—, wherein R^{N1} is independently hydrogen or C₁₋₆alkyl; and
   each B¹ is independently a bond, C(O), C(S), S(O)₂, P(O)(OH), or P(S)(OH).

In another embodiment of Formula (XV-x), each Φ is

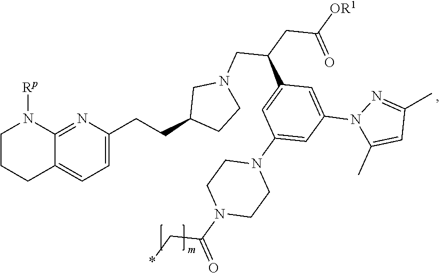

wherein m is an integer selected from 1-10.

In another embodiment of Formula (XV-x), each Φ is

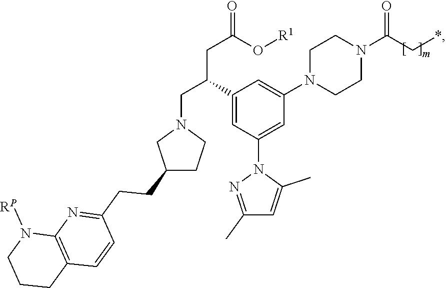

wherein m is an integer selected from 1-10;

In another embodiment of Formula (XV-x), each Φ is

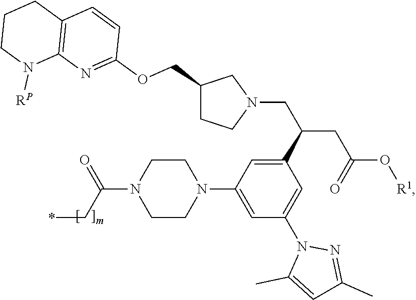

wherein m is an integer selected from 1-10;

In another embodiment of Formula (XV-x), each Φ is

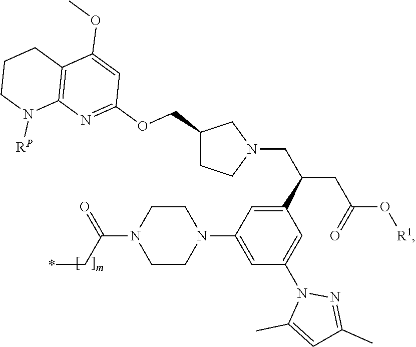

wherein m is an integer selected from 1-10.

In another embodiment of Formula (XV-x), each Φ is

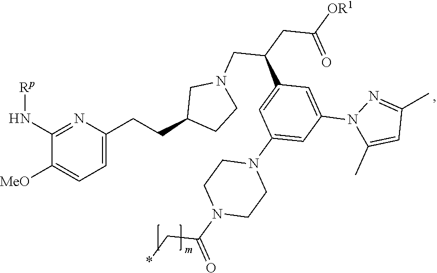

wherein m is an integer selected from 1-10;

In another embodiment of Formula (XV-x) and each of the preceding embodiments thereof,
L⁴ and L⁶ are independently -B¹-A¹- or -A¹-B¹-;
L⁵ is a bond, -B¹-A¹- or -A¹-B¹-;
each G¹ is independently C₁₋₁₀alkyl;
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In another embodiment of Formula (XV-x) and each of the preceding embodiments thereof,
L⁴ is -B¹-A¹- or -A¹-B¹-;
L⁶ is B¹;
L⁵ is a bond, -B¹-A¹- or -A¹-B¹-;
each G¹ is independently C₁₋₁₀alkyl;
each A¹ is independently —O— or —N(H)—, and
each B¹ is independently C(O), S(O)₂, P(O)(OH), or P(S)(OH).

In another embodiment of Formula (XV-x) and each of the preceding embodiments thereof,
-L⁴-G¹-L⁵-G¹-L⁴- is -B¹-C₁₋₁₀alkyl-L⁵-C₁₋₁₀alkyl-B¹-A¹-
L⁵ is a bond, -B¹-A¹- or -A¹-B¹-;
each A¹ is independently —O— or —N(H)—, and
each B¹ is independently C(O), S(O)₂, P(O)(OH), or P(S)(OH).

In another embodiment of Formula (XV-x) and each of the preceding embodiments thereof,
-L⁴-G¹-L⁵-G¹-L⁴- is —C(O)—C₁₋₁₀alkyl-L⁵-C₁₋₁₀alkyl-C(O)N(H)—, wherein L⁵ is a bond, -B¹-A¹- or -A¹-B¹-, wherein A¹ is —O— or —N(H)—, and B¹ is C(O), S(O)₂, P(O)(OH), or P(S)(OH).

In another embodiment of Formula (XV-x) and each of the preceding embodiments thereof,
-L⁴-G¹-L⁵-G¹-L⁴- is —C(O)—C₂₋₂₀alkyl-C(O)N(H)— (e.g., —C(O)—C₆₋₁₂alkyl-C(O)N(H)—, or —C(O)—C₈₋₁₂alkyl-C(O)N(H)—, or —C(O)—C₁₀alkyl-C(O)N(H)—).

In another embodiment, the compound of Formula (XV) is

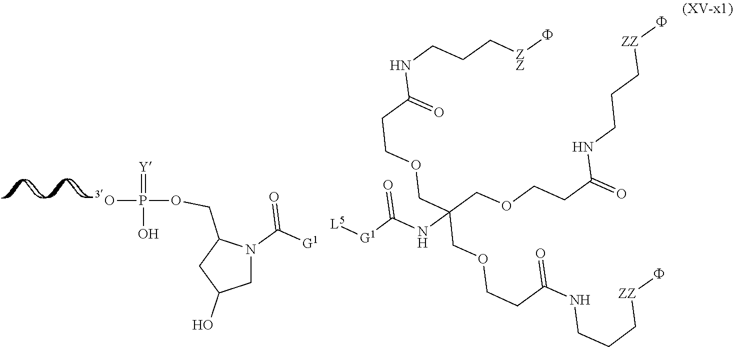

wherein Y′ is O or S;
each ZZ is N(H)C(O) or C(O)N(H);
   each Φ is

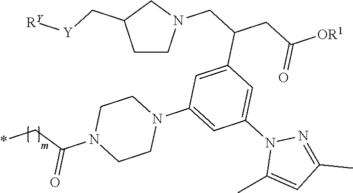

wherein m is an integer selected from 1-10;
L⁵ is a bond or -A¹-B¹-A¹- (e.g., a bond, -B¹-A¹- or -A¹-B¹-; or a bond);
each G¹ is independently C₁₋₁₀alkyl;
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).
   In another embodiment of Formula (XV-x1), each Φ is

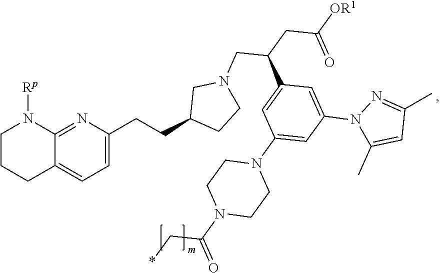

wherein m is an integer selected from 1-10.
In another embodiment of Formula (XV-x1), each Φ is

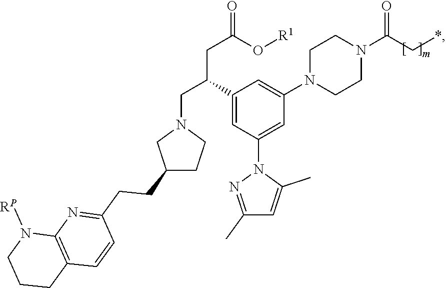

wherein m is an integer selected from 1-10;
In another embodiment of Formula (XV-x1), each Φ is

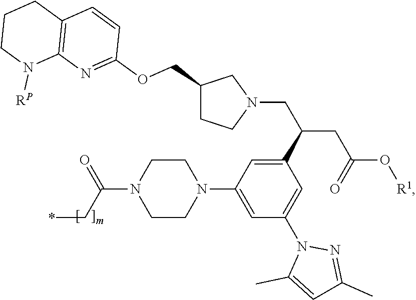

wherein m is an integer selected from 1-10;
In another embodiment of Formula (XV-x1), each Φ is

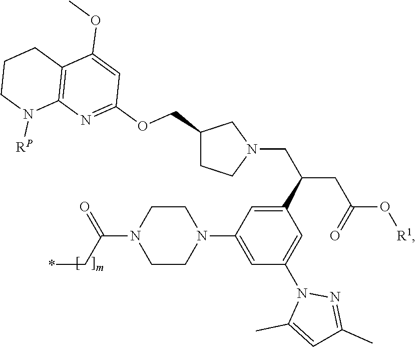

wherein m is an integer selected from 1-10.

In another embodiment of Formula (XV-x1), each Φ is

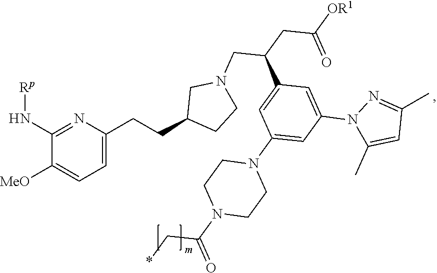

wherein m is an integer selected from 1-10;

In another embodiment, the compound of Formula (XV) is

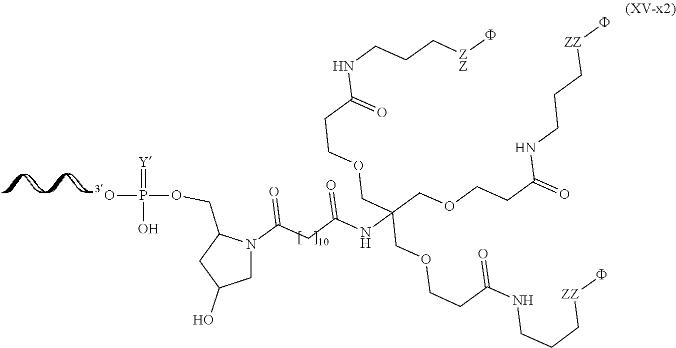

wherein Y′ is O or S;
each ZZ is N(H)C(O) or C(O)N(H);
   each Φ is

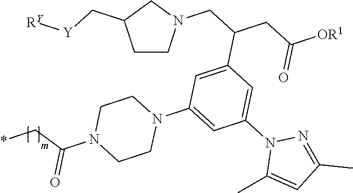

wherein m is an integer selected from 1-10;
L⁵ is a bond or -A¹-B¹-A¹- (e.g., a bond, -B¹-A¹- or -A¹-B¹-; or a bond);
each G¹ is independently C₁₋₁₀alkyl;
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).
   In another embodiment of Formula (XV-x2), each Φ is

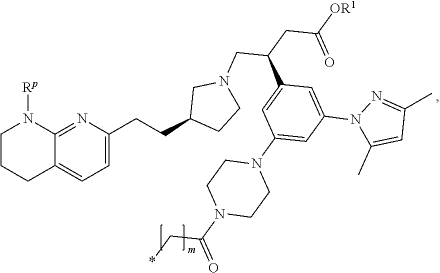

wherein m is an integer selected from 1-10.
In another embodiment of Formula (XV-x2), each Φ is

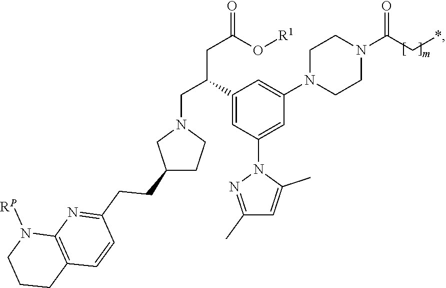

wherein m is an integer selected from 1-10;
In another embodiment of Formula (XV-x2), each Φ is

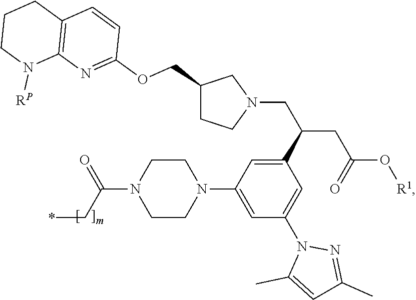

wherein m is an integer selected from 1-10;
In another embodiment of Formula (XV-x2), each Φ is

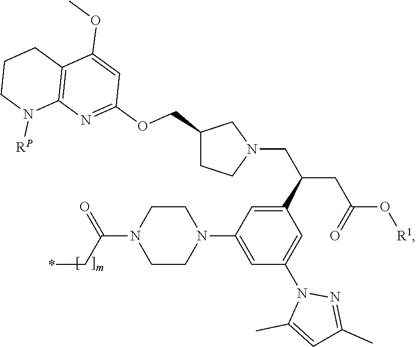

wherein m is an integer selected from 1-10.
In another embodiment of Formula (XV-x2), each Φ is

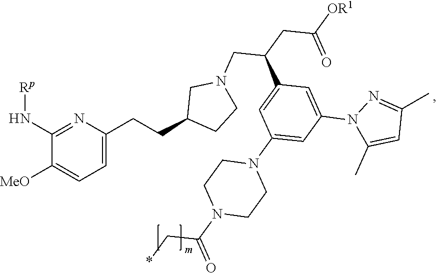

wherein m is an integer selected from 1-10;

In one embodiment of Formula (XV-x), (XV-x1), and (XV-x2), and each of the preceding embodiments thereof, Y′ is O. In another embodiment, Y′ is S.

In one embodiment of Formula (XV-x), (XV-x1), and (XV-x2), and each of the preceding embodiments thereof, R¹ is hydrogen. In another embodiment, R¹ is C₁₋₆alkyl (e.g., methyl or t-butyl).

In one embodiment of Formula (XV-x), (XV-x1), and (XV-x2), and each of the preceding embodiments thereof, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment of Formula (XV-x), (XV-x1), and (XV-x2), and each of the preceding embodiments thereof, Y′ is O and R¹ is hydrogen. In one embodiment, Y′ is O and R¹ is C₁₋₆alkyl (e.g., methyl or t-butyl). In one embodiment, Y′ is S and R¹ is hydrogen. In one embodiment, Y′ is S and R¹ is C₁₋₆alkyl (e.g., methyl ort-butyl).

In one embodiment of Formula (XV-x), (XV-x1), and (XV-x2), and each of the preceding embodiments thereof, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment of Formula (XV-x), (XV-x1), and (XV-x2), and each of the preceding embodiments thereof, Y′ is O, R¹ is hydrogen and R^{P} is hydrogen. In one embodiment, Y′ is S, R¹ is hydrogen and R^{P} is hydrogen. In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In other embodiments of Formula (XV), the compound is of the structure:

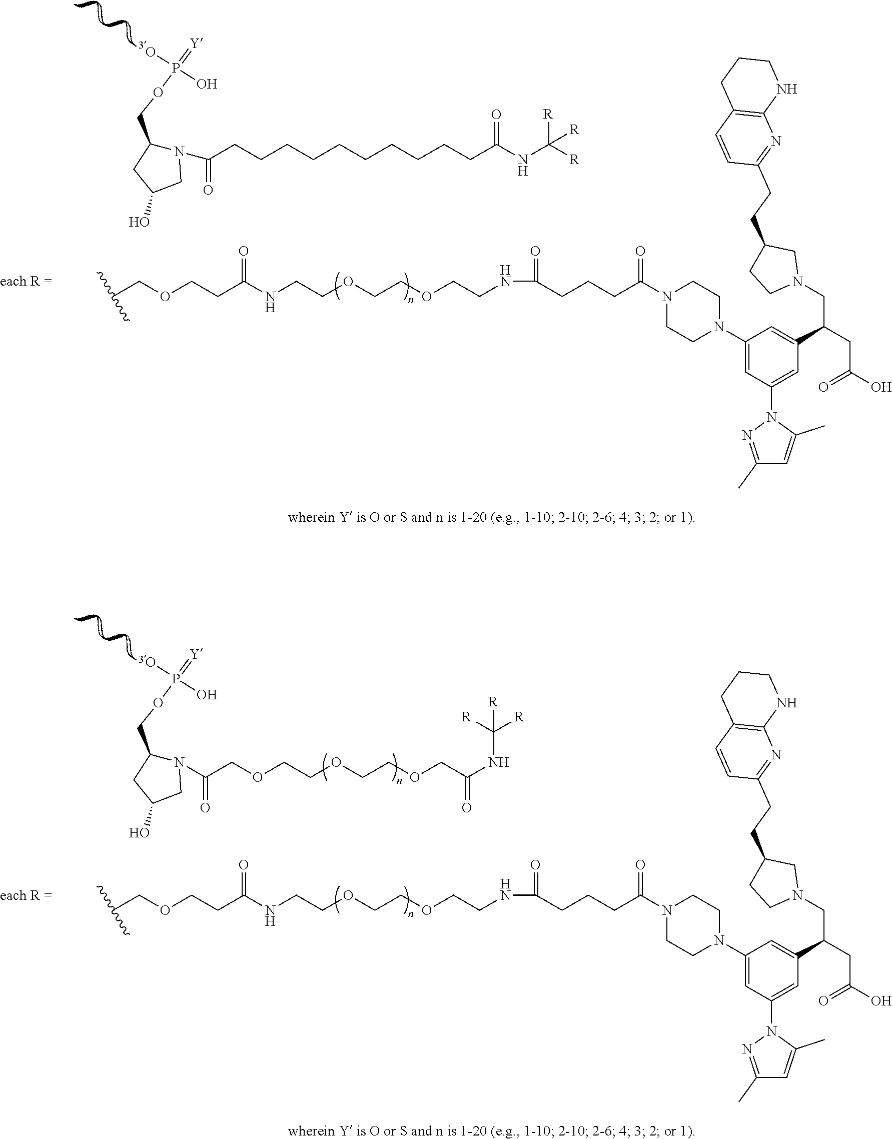

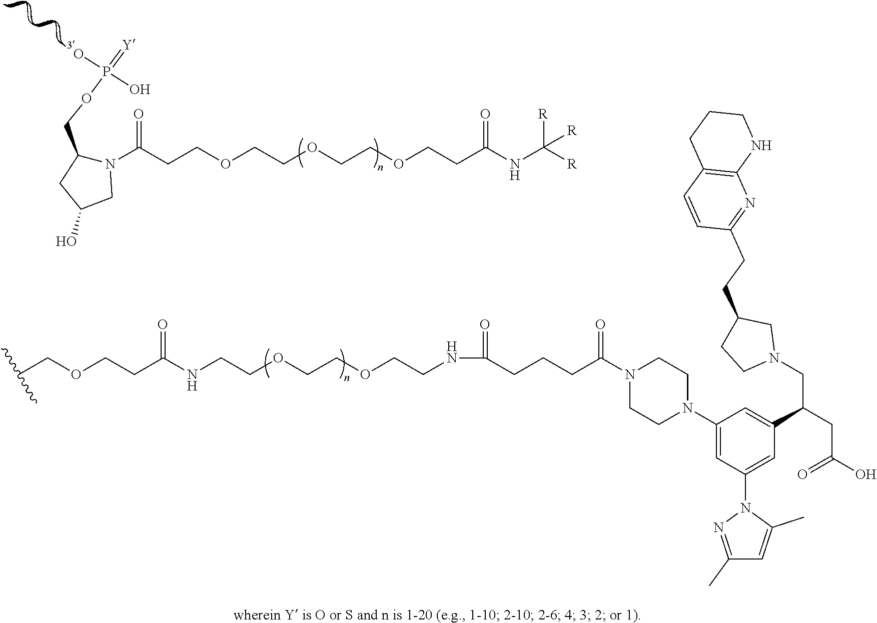

### R^{T1}, Phosphorous Coupling Groups

In another embodiment, R^{P3}, when present, is a hydroxyl protecting group (e.g., according to any one of the preceding embodiments of R^{P3}) and R^{T1} is phosphorous coupling group

In another embodiment, R^{P3}, when present, is a hydroxyl protecting group (e.g., according to any one of the preceding embodiments of R^{P3}) and R^{T1} is phosphorous coupling group of the formula —P(Z)(XV), wherein:
X is selected from the group consisting of
C₁₋₆alkyl (e.g., methyl),
C₁₋₆alkoxyC₁₋₆alkyl (e.g., 3-methoxypropyl),
C₁₋₆alkoxy (e.g., —OCH₃, —OCH₂CH₃, —OCH₂CH₂CH₃, —OCH₂CH(CH₃)₂),
C₁₋₆alkoxy substituted with R (e.g., —OCH₂CH₂CN, —OCH₂CH₂Si(CH₃)₃, —OCH₂CH₂Si(CH₂CH₃)₃

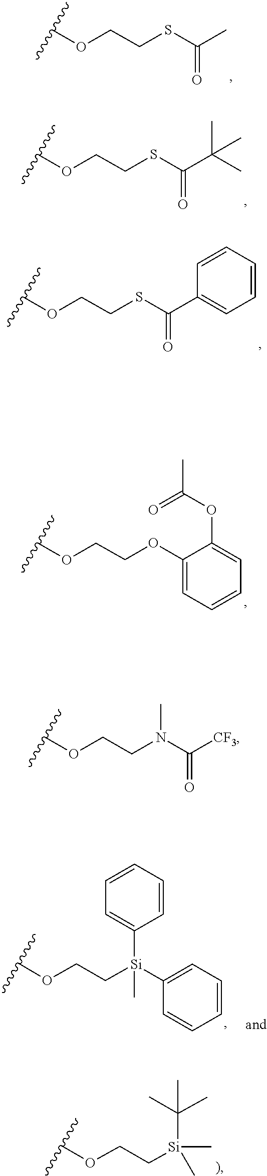

C₂₋₆alkenyloxy (e.g., —OC(H)═CH₂, —OCH₂C(H)═CH₂),
phenoxy optionally substituted with 1, 2, 3, or 4 R groups.

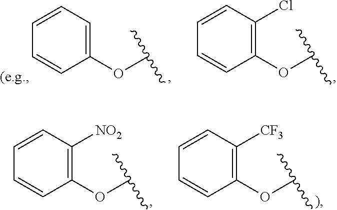 [SMILES: *Oc1ccccc1 ; *Oc1ccccc1Cl ; *Oc1ccccc1[N+](=O)[O-] ; *Oc1ccccc1C(F)(F)F]

benzyloxy optionally substituted with 1, 2, 3, or 4 R groups. (e.g.,

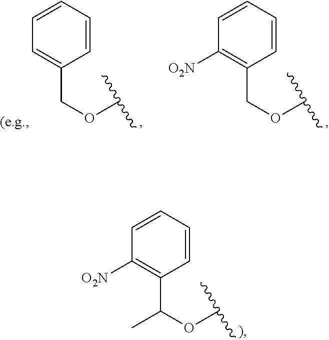 [SMILES: *OCc1ccccc1 ; *OCc1ccccc1[N+](=O)[O-] ; *OC(C)c1ccccc1[N+](=O)[O-]]

and
Z is selected from the group consisting of
   di(C₁₋₆alkyl)amino

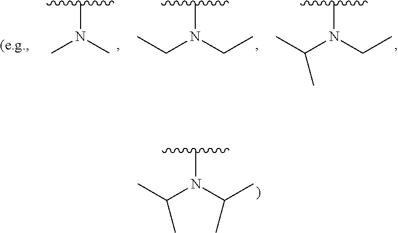

and
heterocyclyl optionally substituted with 1, 2, 3, or 4 R groups.

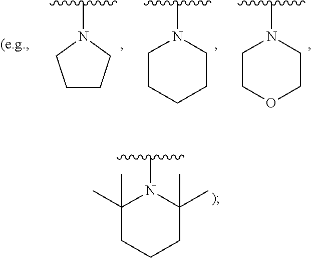

or
X and Z taken together with the phosphorus atom to which they are attached form a cyclic monocyclic or bicyclic heterocyclyl group that is optionally substituted with 1, 2, 3, or 4 R groups.

In another embodiment, R^{P3}, when present, is a hydroxyl protecting group (e.g., according to any one of the preceding embodiments of R^{P3}) and R^{T1} is phosphorous coupling group of the formula,

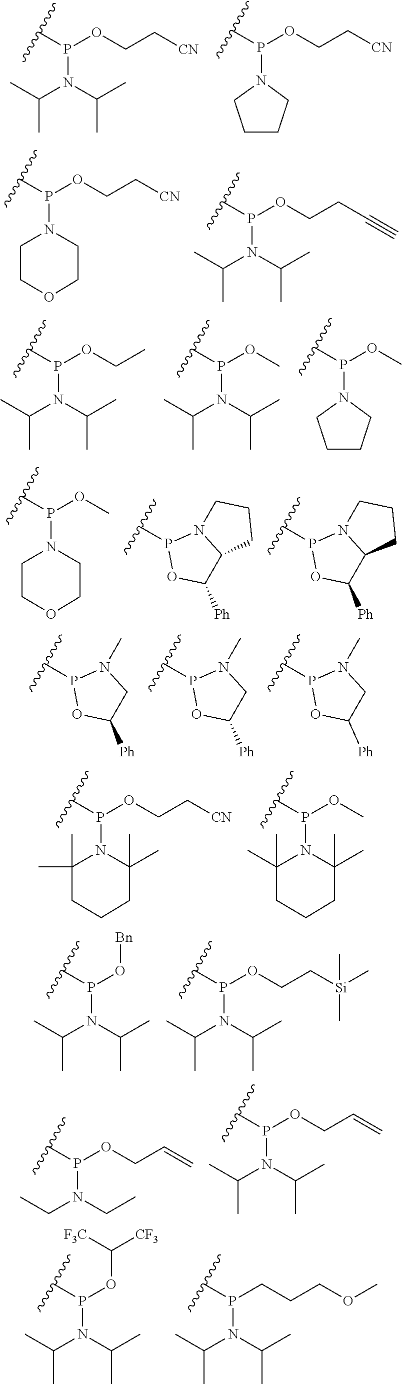

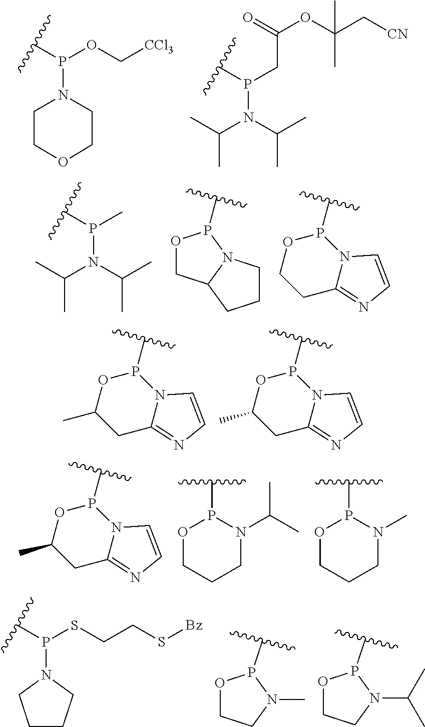

In another embodiment, R^{P3}, when present, is a hydroxyl protecting group (e.g., according to any one of the preceeding embodiments of R^{P3}) and R^{T1} is

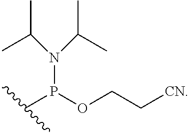 [SMILES: *P(OCCC#N)N(C(C)C)C(C)C]

In another embodiment, R^{P3}, when present, is a hydroxyl protecting group (e.g., according to any one of the preceding embodiments of R^{P3}) and R^{T1} is phosphorous coupling group of the formula

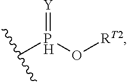

or a salt thereof, wherein Y is O or S; and R^{T2} is hydrogen or —C(O)C₁₋₆alkyl.

In another embodiment, R^{P3}, when present, is a hydroxyl protecting group (e.g., according to any one of the preceding embodiments of R^{P3}) and R^{T1} is phosphorous coupling group of the formula

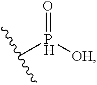

or a salt thereof.

### Solid Supports

In another embodiment, R^{P3}, when present, is a hydroxyl protecting group (e.g., according to any one of the preceding embodiments of R^{P3}) and R^{T1} is -L^{K}-S^{S}, wherein L^{K} is a support linking group and SS is a solid support, —OR^{SS} or —N(R^{SS})₂, or hydrogen, wherein each R^{SS} is independently hydrogen or C₁₋₆alkyl.

In another embodiment, R^{P3}, when present, is a hydroxyl protecting group (e.g., according to any one of the preceding embodiments of R^{P3}) and R^{T1} is -L^{K}-S^{S}, wherein L^{K} is a support linking group and S^{S} is —OR^{SS} or —N(R^{SS})₂.

In another embodiment, R^{P3}, when present, is a hydroxyl protecting group (e.g., according to any one of the preceding embodiments of R^{P3}) and R^{T1} is -L^{K}-S^{S}, wherein L^{K} is a support linking group of the formula: —C(O)(CH₂)ₙC(O)—, wherein n is 1-20; and S^{S} is —OR^{SS} (e.g., —OH).

In another embodiment, R^{P3}, when present, is a hydroxyl protecting group (e.g., according to any one of the preceding embodiments of R^{P3}) and R^{T1} is -L^{K}-S^{S}, wherein L^{K} is a support linking group of the formula: —C(O)CH₂CH₂C(O)—, wherein n is 1-20; and S^{S} is —OR^{SS} (e.g., —OH).

In another embodiment, R^{P3}, when present, is a hydroxyl protecting group (e.g., according to any one of the preceding embodiments of R^{P3}) and R^{T1} is -L^{K}-S^{S}, wherein S^{S} is a solid support.

In another embodiment, R^{P3}, when present, is a hydroxyl protecting group (e.g., according to any one of the preceding embodiments of R^{P3}) and R^{T1} is -L^{K}-S^{S}, wherein S^{S} is a controlled pore glass (CPG),

In another embodiment, R^{P3}, when present, is a hydroxyl protecting group (e.g., according to any one of the preceding embodiments of R^{P3}) and R^{T1} is -L^{K}-S^{S}, wherein S^{S} is a polystyrene (e.g., cross-linked polystyrene).

In an embodiment of each of the preceding, L^{K} is

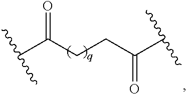

wherein q is 0 or an integer selected from 1-20. In an embodiment of each of the preceding, L^{K} is

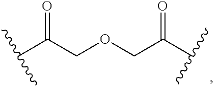 [SMILES: *C(=O)COCC(*)=O]

In an embodiment of each of the preceding, L^{K} is

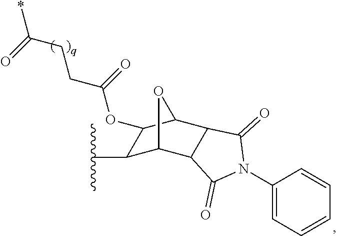

wherein q is 0 or an integer selected from 1-20, and * represents the bond to SS (i.e. to a functional group on the surface of S^{S}).

In an embodiment of each of the preceding, R^{P3}, when present, is a hydroxyl protecting group (e.g., according to any one of the preceding embodiments of R^{P3}) and R^{T1} is

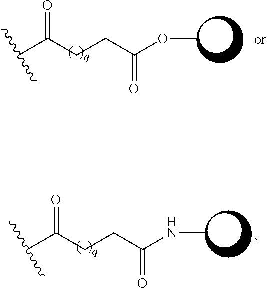

where [symbol] is the solid support.

In an embodiment of each of the preceding, R^{P3}, when present, is a hydroxyl protecting group (e.g. according to any one of the preceding embodiments of R^{P3}) and R^{T1} is

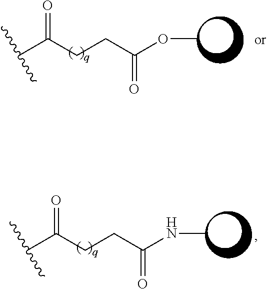

wherein [symbol] is the solid support.

In an embodiment of each of the preceding, R^{P3}, when present, is a hydroxyl protecting group (e.g., according to any one of the preceding embodiments of R^{P3}) and R^{T1} is

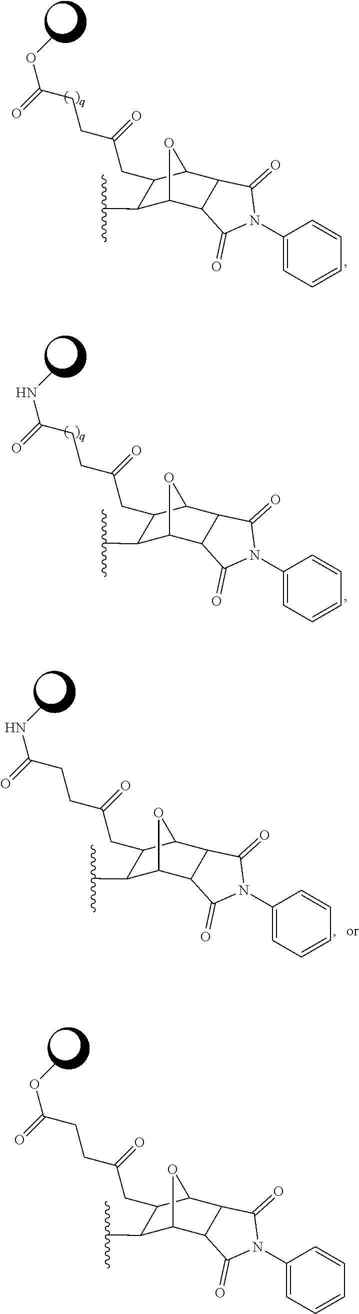 [SMILES: *C1C(CC(=O)CCC(=O)OC)C2OC1C1C(=O)N(c3ccccc3)C(=O)C21 ; *C1C(CC(=O)CCC(=O)NC)C2OC1C1C(=O)N(c3ccccc3)C(=O)C21 ; *C1C(CC(=O)CCC(=O)NC)C2OC1C1C(=O)N(c3ccccc3)C(=O)C21 ; *C1C(CC(=O)CCC(=O)OC)C2OC1C1C(=O)N(c3ccccc3)C(=O)C21]

In another embodiment, the compound of Formula (XV) is

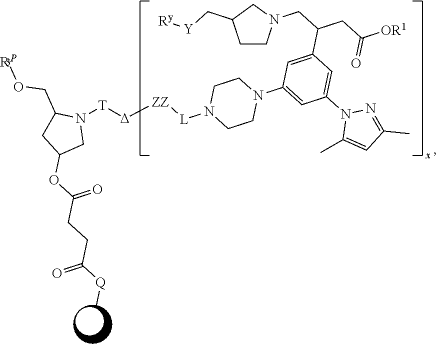

wherein [symbol] represents a solid support; Q is O or NH, and Δ, R^{Y}, Y, R¹, L, T, and ZZ are as defined for Formula (XV) or any embodiment thereof. In one embodiment, R¹ is hydrogen. In another embodiment, R¹ is C₁₋₆alkyl (e.g., methyl or t-butyl). In one embodiment, R^{P3} is hydrogen. In another embodiment, R^{P3} is hydroxyl protecting group (e.g., 4,4′-dimethyoxytrityl (DMTr)).

In another embodiment, the compound of Formula (XV) is

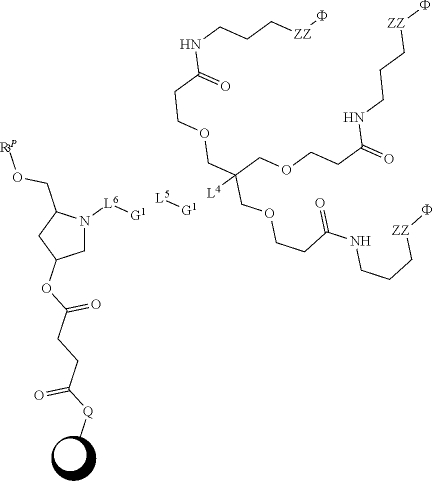

wherein [symbol] represents a solid support; Q is O or NH,
each ZZ is N(H)C(O) or C(O)N(H);
   each Φ is

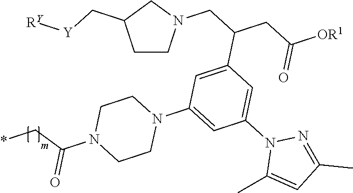

wherein m is an integer selected from 1-10;
L⁴ and L⁶ are independently -B¹-A¹- or -A¹-B¹-;
each L⁵ is a bond or -A¹-B¹-A¹- (e.g., a bond, -B¹-A¹- or -A¹-B¹-; or a bond);
each G¹ is independently C₁₋₁₀alkyl or C₂₋₁₀alkenyl;
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).
   In another embodiment of Formula (XV-z), each Φ is

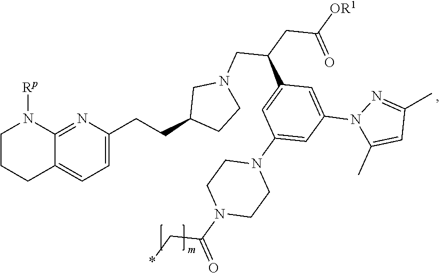

wherein m is an integer selected from 1-10.
In another embodiment of Formula (XV-z), each Φ is

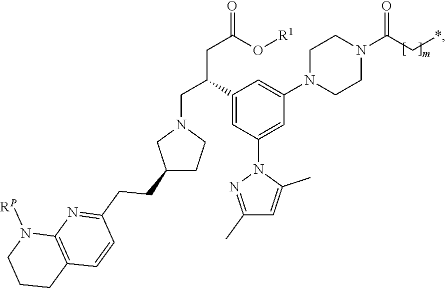

wherein m is an integer selected from 1-10;
In another embodiment of Formula (XV-z), each Φ is

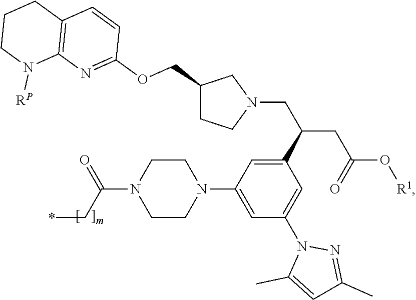

wherein m is an integer selected from 1-10;
In another embodiment of Formula (XV-z), each Φ is

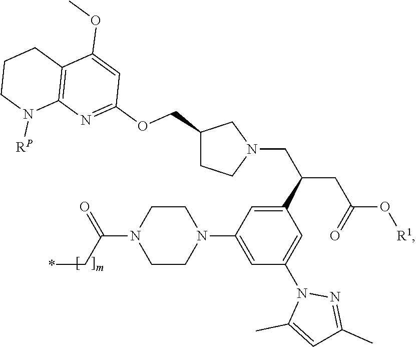

wherein m is an integer selected from 1-10;
In another embodiment of Formula (XV-z), each Φ is

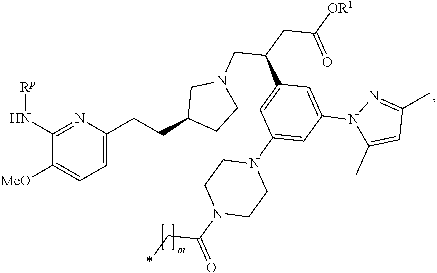

wherein m is an integer selected from 1-10;

In another embodiment of Formula (XV-z) and each of the preceding embodiments thereof,
L⁴ and L⁶ are independently -B¹-A¹- or -A¹-B¹-;
L⁵ is a bond, -B¹-A¹- or -A¹-B¹-;
each G¹ is independently C₁₋₁₀alkyl;
each A¹ is independently a bond, —O—, —S—, or —N(R^{N1})—, wherein R^{N1} is independently hydrogen or C₁₋₆alkyl; and
each B¹ is independently a bond, C(O), C(S), S(O)₂, P(O)(OH), or P(S)(OH).

In another embodiment of Formula (XV-z) and each of the preceding embodiments thereof,
L⁴ is -B¹-A¹- or -A¹-B¹-;
L⁶ is B¹;
L⁵ is a bond, -B¹-A¹- or -A¹-B¹-;
each G¹ is independently C₁₋₁₀alkyl;
each A¹ is independently —O— or —N(H)—, and
each B¹ is independently C(O), S(O)₂, P(O)(OH), or P(S)(OH).

In another embodiment of Formula (XV-z) and each of the preceding embodiments thereof,
-L⁴-G¹-L⁵-G¹-L⁴- is -B¹-C₁₋₁₀alkyl-L⁵-C₁₋₁₀alkyl-B¹-A¹-
L⁵ is a bond, -B¹-A¹- or -A¹-B¹-;
each A¹ is independently —O— or —N(H)—, and
each B¹ is independently C(O), S(O)₂, P(O)(OH), or P(S)(OH).

In another embodiment of Formula (XV-z) and each of the preceding embodiments thereof,
-L⁴-G¹-L⁵-G¹-L⁴- is —C(O)—C₁₋₁₀alkyl-L⁵-C₁₋₁₀alkyl-C(O)N(H)—, wherein L⁵ is a bond, -B¹-A¹- or -A¹-B¹-, wherein A¹ is —O— or —N(H)—, and B¹ is C(O), S(O)₂, P(O)(OH), or P(S)(OH).

In another embodiment of Formula (XV-z) and each of the preceding embodiments thereof,
-L⁴-G¹-L⁵-G¹-L⁴- is —C(O)—C₂₋₂₀alkyl-C(O)N(H)— (e.g., —C(O)—C₆₋₁₂alkyl-C(O)N(H)—, or —C(O)—C₈₋₁₂alkyl-C(O)N(H)—, or —C(O)—C₁₀alkyl-C(O)N(H)—).

In one embodiment of Formula (XV-z) and each of the preceding embodiments thereof, Y′ is O. In another embodiment, Y′ is S.

In one embodiment of Formula (XV-z) and each of the preceding embodiments thereof, R¹ is hydrogen. In another embodiment, R¹ is C₁₋₆alkyl (e.g., methyl or t-butyl).

In one embodiment of Formula (XV-z) and each of the preceding embodiments thereof, R^{P} is hydrogen. In another embodiment, R^{P} is a nitrogen protecting group (e.g., t-butoxycarbonyl (Boc), benzyloxycarbonyl (Cbz) or phenoxyacetyl (pac)).

In one embodiment of Formula (XV-z) and each of the preceding embodiments thereof, Y′ is O and R¹ is hydrogen. In one embodiment, Y′ is O and R¹ is C₁₋₆alkyl (e.g., methyl or t-butyl). In one embodiment, Y′ is S and R¹ is hydrogen. In one embodiment, Y′ is S and R¹ is C₁₋₆alkyl (e.g., methyl or t-butyl).

In one embodiment of Formula (XV-z) and each of the preceding embodiments thereof, Y′ is O and R^{P} is hydrogen. In one embodiment, Y′ is O and R^{P} is a nitrogen protecting group (e.g., t-butoxycarbonyl (Boc), benzyloxycarbonyl (Cbz) or phenoxyacetyl (pac)). In one embodiment, Y′ is S and R^{P} is hydrogen. In one embodiment, Y′ is S and R^{P} is a nitrogen protecting group (e.g., t-butoxycarbonyl (Boc), benzyloxycarbonyl (Cbz) or phenoxyacetyl (pac)).

In one embodiment of Formula (XV-z) and each of the preceding embodiments thereof, Y′ is O, R¹ is hydrogen and R^{P} is hydrogen. In one embodiment, Y′ is S, R¹ is hydrogen and R^{P} is hydrogen.

In one embodiment, Y′ is O, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is S, R¹ is hydrogen and R^{P} is a nitrogen protecting group (e.g., t-butoxycarbonyl (Boc), benzyloxycarbonyl (Cbz) or phenoxyacetyl (pac)). In one embodiment, Y′ is O, R¹ is C₁₋₆alkyl (e.g., methyl or t-butyl) and R^{P} is hydrogen. In one embodiment, Y′ is S, R¹ is C₁₋₆alkyl (e.g., methyl or t-butyl) and R^{P} is hydrogen. In one embodiment, Y′ is O, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)). In one embodiment, Y′ is S, R¹ is C₁₋₆alkyl (e.g., methyl or t-butyl) and R^{P} is a nitrogen protecting group (e.g., t-butoxycarbonyl (Boc), benzyloxycarbonyl (Cbz) or phenoxyacetyl (pac)).

In another embodiment, the compound of Formula (XV) is selected from the group consisting of:

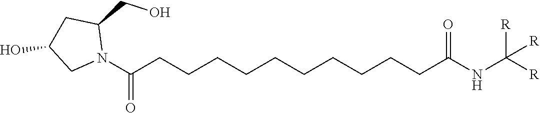

each R =

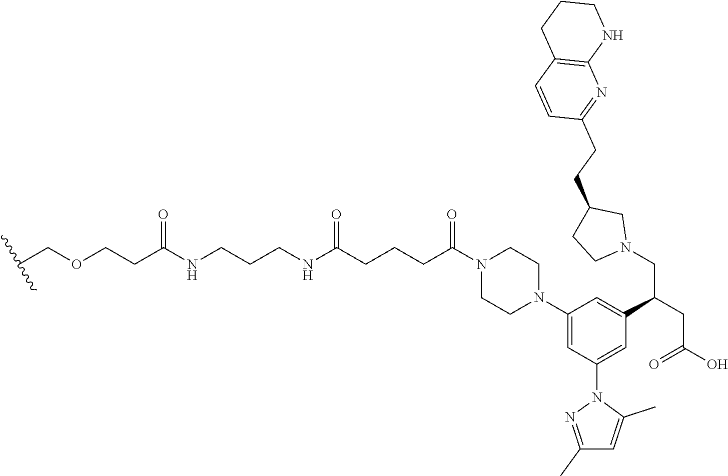 [SMILES: *COCCC(=O)NCCCNC(=O)CCCC(=O)N1CCN(c2cc([C@H](CC(=O)O)CN3CC[C@@H](CCc4ccc5c(n4)NCCC5)C3)cc(-n3nc(C)cc3C)c2)CC1]

(2S,4R)-1-[23-((5-(4-(3-((S)-1-carboxy-3-((R)-3-(2-(5,6,7,8-tetrahydro-1,8-naphthyridin-2-
yl)ethyl)pyrrolidine-1-yl)propan-2-yl)-5-(3,5-dimethyl-1H-pyrazol-1-yl)phenyl)piperazin-1-
yl)-5-oxopentan-1-oyl)amino)-14,14-bis[3-(3-((5-(4-(3-((S)-1-carboxy-3-((R)-3-(2-(5,6,7,8-
tetrahydro-1,8-naphthyridin-2-yl)ethyl)pyrrolidine-1-yl)propan-2-yl)-5-(3,5-dimethyl-1H-
pyrazol-1-yl)phenyl)piperazin-1-yl)-5-oxopentan-1-oyl)amino)propylamino)-3-oxo-
propoxymethyl]-1,12,19-trioxo-16-oxa-13,20-diazatricos-1-yl]-4-hydroxy-2-
hydroxymethylpyrrolidine

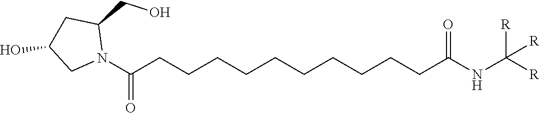

each R =

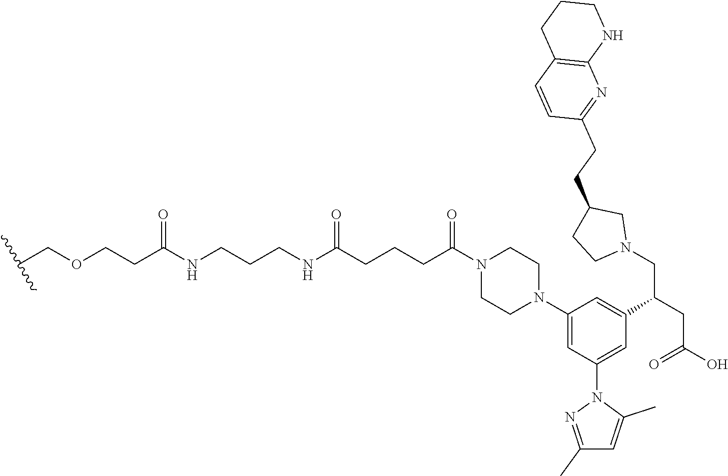 [SMILES: *COCCC(=O)NCCCNC(=O)CCCC(=O)N1CCN(c2cc([C@@H](CC(=O)O)CN3CC[C@@H](CCc4ccc5c(n4)NCCC5)C3)cc(-n3nc(C)cc3C)c2)CC1]

(2S,4R)-1-[23-((5-(4-(3-((R)-1-carboxy-3-((R)-3-(2-(5,6,7,8-tetrahydro-1,8-naphthyridin-2-
yl)ethyl)pyrrolidin-1-yl)propan-2-yl)-5-(3,5-dimethyl-1H-pyrazol-1-yl)phenyl)piperazin-1-yl)-
5-oxopentan-1-oyl)amino)-14,14-bis[3-(3-((5-(4-(3-((R)-1-carboxy-3-((R)-3-(2-(5,6,7,8-
tetrahydro-1,8-naphthyridin-2-yl)ethyl)pyrrolidin-1-yl)propan-2-yl)-5-(3,5-dimethyl-1H-
pyrazol-1-yl)phenyl)piperazin-1-yl)-5-oxopentan-1-oyl)amino)propylamino)-3-oxo-
propoxymethyl]-1,12,19-trioxo-16-oxa-13,20-diazatricos-1-yl]-4-hydroxy-2-
hydroxymethylpyrrolidine

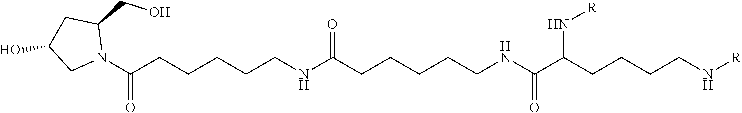

each R =

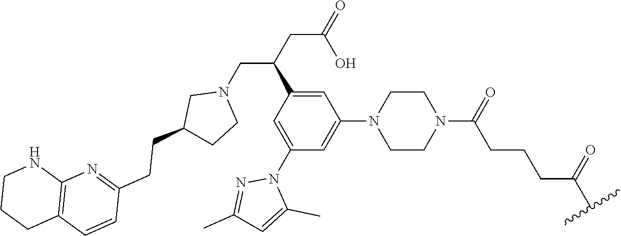 [SMILES: *C(=O)CCCC(=O)N1CCN(c2cc([C@H](CC(=O)O)CN3CC[C@@H](CCc4ccc5c(n4)NCCC5)C3)cc(-n3nc(C)cc3C)c2)CC1]

(2S,4R)-1-(6-(6-(2,6-di((5-(4-(3-((S)-1-carboxy-3-((R)-3-(2-(5,6,7,8-tetrahydro-1,8-
naphthyridin-2-yl)ethyl)pyrrolidin-1-yl)propan-2-yl)-5-(3,5-dimethyl-1H-pyrazol-1-
yl)phenyl)piperazin-1-yl)-5-oxopentan-1-oyl)amino)-(2S)-hexan-1-oylamino)hexan-1-
oylamino)hexan-1-oyl)-2-hydroxymethyl-4-hydroxypyrrolidine

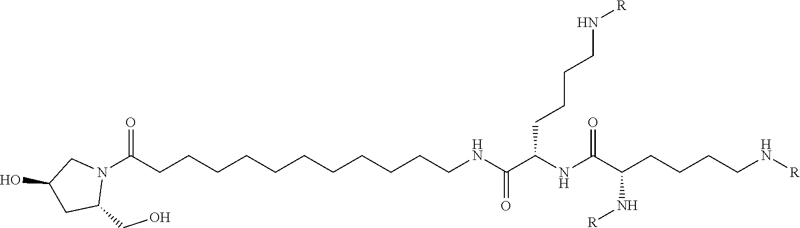

each R =

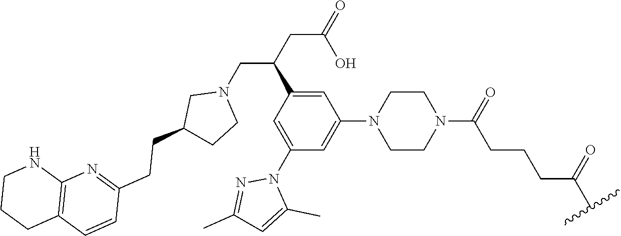 [SMILES: *C(=O)CCCC(=O)N1CCN(c2cc([C@H](CC(=O)O)CN3CC[C@@H](CCc4ccc5c(n4)NCCC5)C3)cc(-n3nc(C)cc3C)c2)CC1]

(2S,4R)-1-(12-(((N-(5-(4-(3-((S)-1-carboxy-3-((R)-3-(2-(5,6,7,8-tetrahydro-1,8-naphthyridin-
2-yl)ethyl)pyrrolidin-1-yl)propan-2-yl)-5-(3,5-dimethyl-1H-pyrazol-1-yl)phenyl)piperazin-1-
yl)-5-oxopentan-1-oyl)-N^{ε}-(5-(4-(3-((S)-1-carboxy-3-((R)-3-(2-(5,6,7,8-tetrahydro-1,8-
naphthyridin-2-yl)ethyl)pyrrolidin-1-yl)propan-2-yl)-5-(3,5-dimethyl-1H-pyrazol-1-
yl)phenyl)piperazin-1-yl)-5-oxopentan-1-oyl)lysyl)-N^{ε}-(5-(4-(3-((S)-1-carboxy-3-((R)-3-(2-
(5,6,7,8-tetrahydro-1,8-naphthyridin-2-yl)ethyl)pyrrolidin-1-yl)propan-2-yl)-5-(3,5-dimethyl-
1H-pyrazol-1-yl)phenyl)piperazin-1-yl)-5-oxopentan-1-oyl)lysyl)amino)dodecan-1-oyl)-2-
hydroxymethyl-4-hydroxypyrrolidine

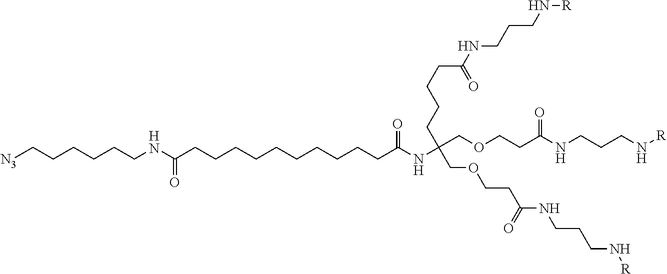

each R =

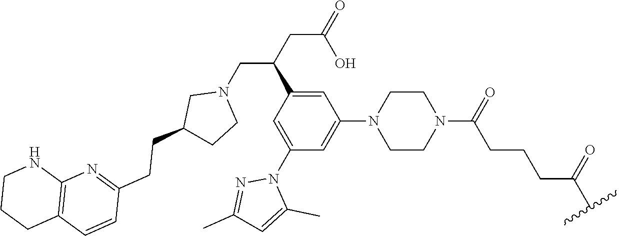 [SMILES: *C(=O)CCCC(=O)N1CCN(c2cc([C@H](CC(=O)O)CN3CC[C@@H](CCc4ccc5c(n4)NCCC5)C3)cc(-n3nc(C)cc3C)c2)CC1]

6-((23-((5-(4-(3-((S)-1-carboxy-3-((R)-3-(2-(5,6,7,8-tetrahydro-1,8-naphthyridin-2-
yl)ethyl)pyrrolidin-1-yl)propan-2-yl)-5-(3,5-dimethyl-1H-pyrazol-1-yl)phenyl)piperazin-1-yl)-
5-oxopentan-1-oyl)amino)-14,14-bis[3-(3-((5-(4-(3-((S)-1-carboxy-3-((R)-3-(2-(5,6,7,8-
tetrahydro-1,8-naphthyridin-2-yl)ethyl)pyrrolidin-1-yl)propan-2-yl)-5-(3,5-dimethyl-1H-
pyrazol-1-yl)phenyl)piperazin-1-yl)-5-oxopentan-1-oyl)amino)propylamino)-3-oxo-
propoxymethyl]-1,12,19-trioxo-16-oxa-13,20-diazatricos-1-yl)amino)-1-azidohexane

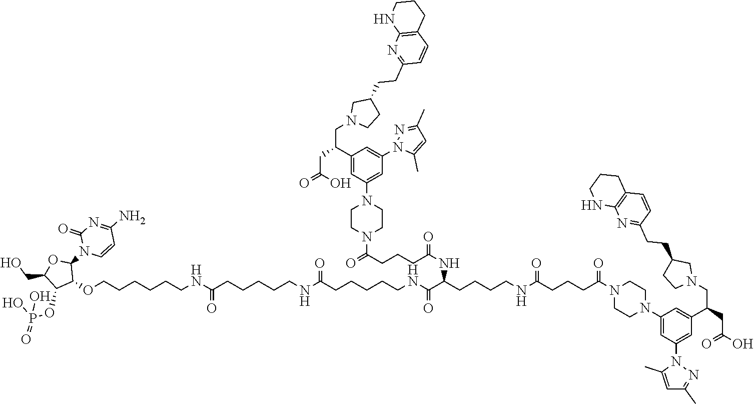 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCC[C@H](NC(=O)CCCC(=O)N4CCN(c5cc([C@H](CC(=O)O)CN6CC[C@@H](CCc7ccc8c(n7)NCCC8)C6)cc(-n6nc(C)cc6C)c5)CC4)C(=O)NCCCCCC(=O)NCCCCCC(=O)NCCCCCCO[C@@H]4[C@H](OP(=O)(O)O)[C@@H](CO)O[C@H]4n4ccc(N)nc4=O)CC3)c2)n1]

2′-O-(6-(6-(6-(N,N^{ε}-bis(5-(4-(3-((S)-1-carboxy-3-((R)-3-(2-(5,6,7,8-tetrahydro-1,8-
naphthyridin-2-yl)ethyl)pyrrolidin-1-yl)propan-2-yl)-5-(3,5-dimethyl-1H-pyrazol-1-
yl)phenyl)piperazin-1-yl)-5-oxopentan-1-oyl)-L-lysyl)aminohexan-1-oylamino)hexan-1-
oylamino)hex-1-yl)-cytidine 3′-phosphate;
and salts thereof.

### Synthetic Intermediates

In another aspect, compounds are provided that are suitable for preparing the various ligands described herein. In one embodiment, the compound is of the Formula (VI), (VI-a) through (VI-e), and (VII),

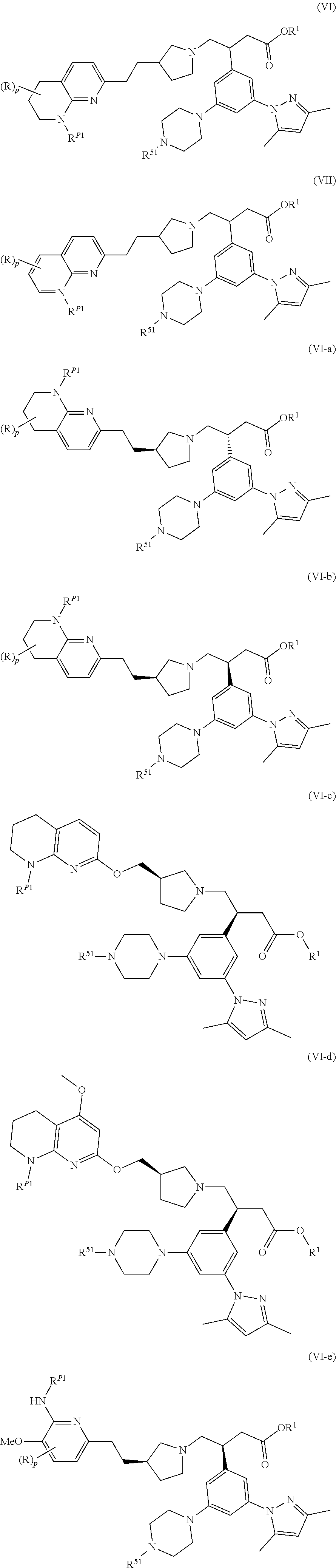

or a salt thereof, wherein
R¹ is hydrogen or C₁₋₆alkyl (e.g., methyl or tert-butyl);
R¹ and R⁵¹ are independently hydrogen or a nitrogen-protecting group;
and
each R group is independently selected from the group consisting of R′, C₁₋₆alkyl, C₁₋₆haloalkyl, C₂₋₆alkenyl, C₂₋₆alkynyl, C₃₋₈cycloalkyl, heterocyclyl, aryl, heteroaryl, C₃₋₈cycloalkylC₁₋₆alkyl, heterocyclylC₁₋₆alkyl, aryl C₁₋₆alkyl, heteroarylC₁₋₆alkyl, each of which, other than R′, is optionally substituted with 1, 2, or 3 R′ groups, wherein
   each R′ is independently halogen, cyano, azido, nitro, —N(R^{b})₂, —O(R^{a}), —S(R⁰), —C(O)OR⁰, —C(O)R⁰, —C(O)N(R⁰)₂, —C(NR⁰)OR⁰, —C(NR⁰)R⁰, —C(NR⁰)N(R⁰)₂, —C(S)OR⁰, —C(S)R⁰, —C(S)N(R⁰)₂, —S(O)₂R⁰, —S(O)₂OR⁰, —S(O)₂N(R⁰)₂, —N(R⁰)C(O)OR, —N(R⁰)C(O)R⁰, —N (R⁰)C(O)N(R⁰)₂, —N(R⁰)S(O)₂R⁰, —N(R⁰)S(O)₂OR⁰, —N(R⁰)S(O)₂N(R⁰)₂, —OC(O)OR, —OC(O)R⁰, —OC(O)N(R⁰)₂, —OS(O)₂R⁰, —OS(O)₂OR⁰, —OS(O)₂N(R⁰)₂, or —SC(O)R⁰, wherein
      each R⁰ is independently hydrogen or C₁₋₆alkyl; each R^{a} is independently hydrogen, C₁₋₆alkyl, or a hydroxyl protecting group; and each R^{b} is independently hydrogen, C₁₋₆alkyl, or a nitrogen protecting group.

In one embodiment of Formulae (VI), (VI-a) through (VI-e), and (VII),
(a) R¹ is hydrogen;
(b) R¹ is C₁₋₆alkyl (e.g., methyl or t-butyl);
(c) R¹ is hydrogen;
(d) R¹ is a nitrogen protecting group;
(e) R⁵¹ is hydrogen; or
(f) R⁵¹ is a nitrogen protecting group.

In one embodiment of Formulae (VI), (VI-a) through (VI-d), and (VII),
(a) R¹ is hydrogen and R¹ is a nitrogen protecting group;
(b) R¹ is hydrogen and R¹ is hydrogen;
(c) R¹ is C₁₋₆alkyl (e.g., methyl or t-butyl), and R¹ is a nitrogen protecting group;
(d) R¹ is C₁₋₆alkyl (e.g., methyl or t-butyl), and R¹ is hydrogen;
(e) R¹ is hydrogen and R⁵¹ is hydrogen;
(f) R¹ is hydrogen and R⁵¹ is a nitrogen protecting group;
(g) R¹ is C₁₋₆alkyl (e.g., methyl or t-butyl), and R¹ is hydrogen;
(h) R¹ is C₁₋₆alkyl (e.g., methyl or t-butyl), and R¹ is a nitrogen protecting group;
(i) R¹ is a nitrogen protecting group. and R⁵¹ is hydrogen;
(j) R¹ is a nitrogen protecting group. and R⁵¹ is a nitrogen protecting group;
(k) R¹ is hydrogen, and R⁵¹ is hydrogen; or
(l) R¹ is hydrogen, and R⁵¹ is a nitrogen protecting group.

In another embodiment of Formulae (VI), (VI-a) through (VI-d), and (VII),
(a) R¹ is hydrogen, R⁵¹ is hydrogen, and R¹ is hydrogen;
(b) R¹ is hydrogen, R⁵¹ is hydrogen, and R¹ is a nitrogen protecting group;
(c) R¹ is hydrogen, R⁵¹ is a nitrogen protecting group, and R¹ is hydrogen;
(d) R¹ is hydrogen, R⁵¹ is a nitrogen protecting group, and R¹ is a nitrogen protecting group;
(e) R¹ is C₁₋₆alkyl (e.g., methyl ort-butyl), R¹ is hydrogen, and R¹ is hydrogen;
(f) R¹ is C₁₋₆alkyl (e.g., methyl ort-butyl), R¹ is hydrogen, and R¹ is a nitrogen protecting group;
(g) R¹ is C₁₋₆alkyl (e.g., methyl or t-butyl), R⁵¹ is a nitrogen protecting group, and R^{P1} is hydrogen; or
(h) R¹ is C₁₋₆alkyl (e.g., methyl ort- butyl), R⁵¹ is a nitrogen protecting group, and R^{P1} is a nitrogen protecting group.

In one embodiment, the compound of Formula (VI) is selected from the group consisting of:

| | |
|---|---|
| 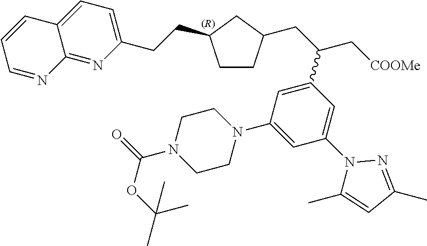 | tert-butyl (R)-4-(3-(1-(3-(2-(1,8-naphthyridin-2- yl)ethyl)pyrrolidin-1-yl)-4-methoxy-4-oxobutan-2-yl)-5-(3,5- dimethyl-1H-pyrazol-1-yl)phenyl)piperazine-1-carboxylate |
| 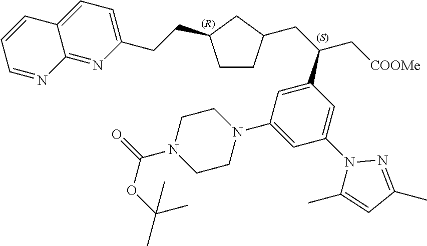 | Tert-butyl 4-(3-((S)-1-((R)-3-(2-(1,8-naphthyridin-2- yl)ethyl)pyrrolidin-1-yl)-4-methoxy-4-oxobutan-2-yl)-5-(3,5- dimethyl-1H-pyrazol-1-yl)phenyl)piperazine-1-carboxylate |
| 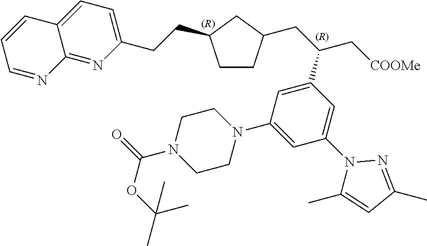 | tert-butyl 4-(3-((R)-1-((R)-3-(2-(1,8-naphthyridin-2- yl)ethyl)pyrrolidin-1-yl)-4-methoxy-4-oxobutan-2-yl)-5-(3,5- dimethyl-1H-pyrazol-1-yl)phenyl)piperazine-1-carboxylate |
| 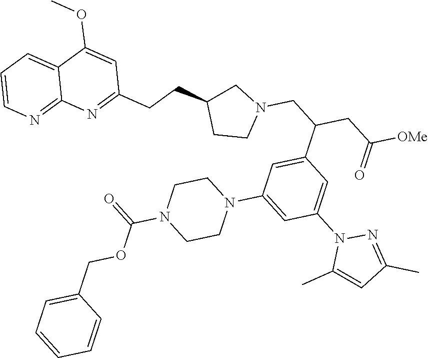 [SMILES: COC(=O)CC(CN1CC[C@@H](CCc2cc(OC)c3cccnc3n2)C1)c1cc(N2CCN(C(=O)OCc3ccccc3)CC2)cc(-n2nc(C)cc2C)c1] | benzyl 4-(3-(3,5-dimethyl-1H-pyrazol-1-yl)-5-(4-methoxy-1- ((R)-3-(2-(4-methoxy-1,8-naphthyridin-2-yl)ethyl)pyrrolidin-1- yl)-4-oxobutan-2-yl)phenyl)piperazine-1-carboxylate |
| 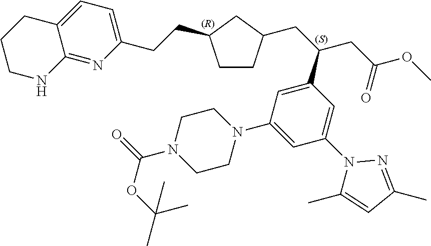 | tert-butyl 4-(3-(3,5-dimethyl-1H-pyrazol-1-yl)-5-((S)-4- methoxy-4-oxo-1-((R)-3-(2-(5,6,7,8-tetrahydro-1,8- naphthyridin-2-yl)ethyl)pyrrolidin-1-yl)butan-2- yl)phenyl)piperazine-1-carboxylate |
| 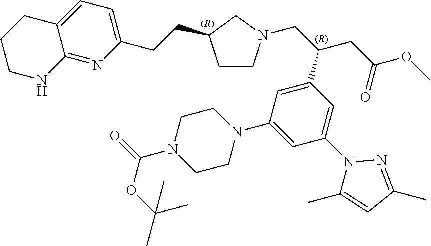 | tert-butyl 4-(3-(3,5-dimethyl-1H-pyrazol-1-yl)-5-((R)-4- methoxy-4-oxo-1-((R)-3-(2-(5,6,7,8-tetrahydro-1,8- naphthyridin-2-yl)ethyl)pyrrolidin-1-yl)butan-2- yl)phenyl)piperazine-1-carboxylate |
| 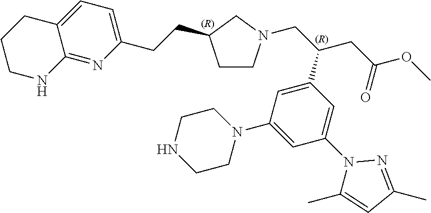 | methyl (R)-3-(3-(3,5-dimethyl-1H-pyrazol-1-yl)-5-(piperazin- 1-yl)phenyl)-4-((R)-3-(2-(5,6,7,8-tetrahydro-1,8-naphthyridin- 2-yl)ethyl)pyrrolidin-1-yl)butanoate |
| 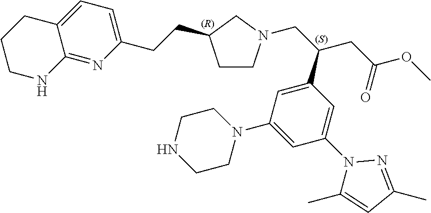 | methyl (S)-3-(3-(3,5-dimethyl-1H-pyrazol-1-yl)-5-(piperazin- 1-yl)phenyl)-4-((R)-3-(2-(5,6,7,8-tetrahydro-1,8-naphthyridin- 2-yl)ethyl)pyrrolidin-1-yl)butanoate |
| 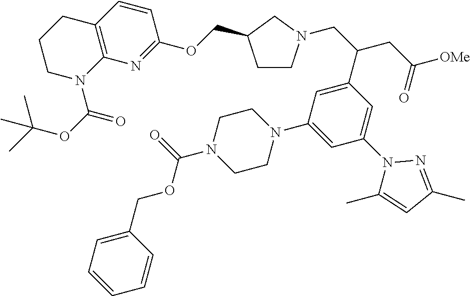 [SMILES: COC(=O)CC(CN1CC[C@@H](COc2ccc3c(n2)N(C(=O)OC(C)(C)C)CCC3)C1)c1cc(N2CCN(C(=O)OCc3ccccc3)CC2)cc(-n2nc(C)cc2C)c1] | tert-butyl 7-(((3R)-1-(2-(3-(4-((benzyloxy)carbonyl)piperazin- 1-yl)-5-(3,5-dimethyl-1H-pyrazol-1-yl)phenyl)-4-methoxy-4- oxobutyl)pyrrolidin-3-yl)methoxy)-3,4-dihydro-1,8- naphthyridine-1(2H)-carboxylate |
| 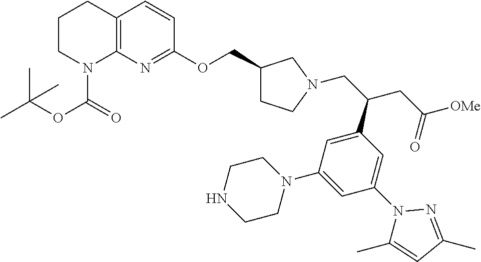 [SMILES: COC(=O)C[C@H](CN1CC[C@@H](COc2ccc3c(n2)N(C(=O)OC(C)(C)C)CCC3)C1)c1cc(N2CCNCC2)cc(-n2nc(C)cc2C)c1] | tert-butyl 7-(((R)-1-((S)-2-(3-(3,5-dimethyl-1H-pyrazol-1-yl)- 5-(piperazin-1-yl)phenyl)-4-methoxy-4-oxobutyl)pyrrolidin-3- yl)methoxy)-3,4-dihydro-1,8-naphthyridine-1(2H)-carboxylate |
| 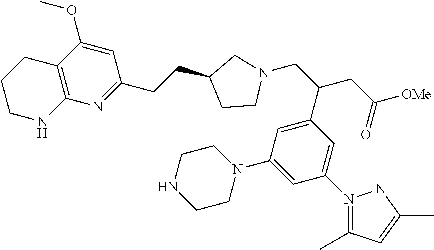 | methyl (S)-3-(3-(3,5-dimethyl-1H-pyrazol-1-yl)-5-(piperazin- 1-yl)phenyl)-4-((R)-3-(2-(4-methoxy-5,6,7,8-tetrahydro-1,8- naphthyridin-2-yl)ethyl)pyrrolidin-1-yl)butanoate |
| 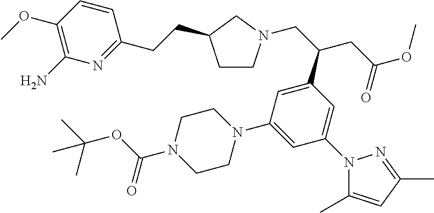 | tert-butyl 4-(3-((S)-1-((R)-3-(2-(6-amino-5-methoxypyridin-2- yl)ethyl)pyrrolidin-1-yl)-4-methoxy-4-oxobutan-2-yl)-5-(3,5- dimethyl-1H-pyrazol-1-yl)phenyl)piperazine-1-carboxylate |
| 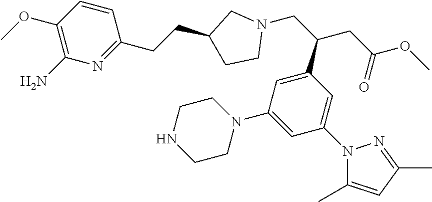 | methyl (S)-4-((R)-3-(2-(6-amino-5-methoxypyridin-2- yl)ethyl)pyrrolidin-1-yl)-3-(3-(3,5-dimethyl-1H-pyrazol-1-yl)- 5-(piperazin-1-yl)phenyl)butanoate |

### Process for Preparing an Oligonucleotide Conjugate

In some embodiments, the present disclosure also provides a process for preparing an oligonucleotide conjugate, comprising contacting an oligonucleotide comprising at least one functional group that is a first member of a reactive pair with a compound of the Formula (IV) or Formula (V) as defined above, wherein the compound comprises a Z or Z⁰ group, according to any of the preceding embodiments, where the Z or Z⁰ group is the second member of the reactive pair, under conditions suitable for forming a covalent linkage between the first member and the second member of the reactive pair.

In some embodiments, the first member of a reactive pair is an azide or

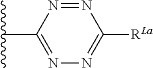

and Z comprises a terminal alkyne

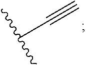 [SMILES: *C#C]

In some embodiments, the first member of a reactive pair is an azide or

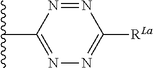

and Z comprises a cyclooctyne, dibenzocyclooctyne, dibenzoazacyclooctyne or trans-cyclooctene, such as,

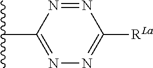

In some embodiments, Z is an azide or

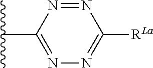

and the first member of a reactive pair comprises:

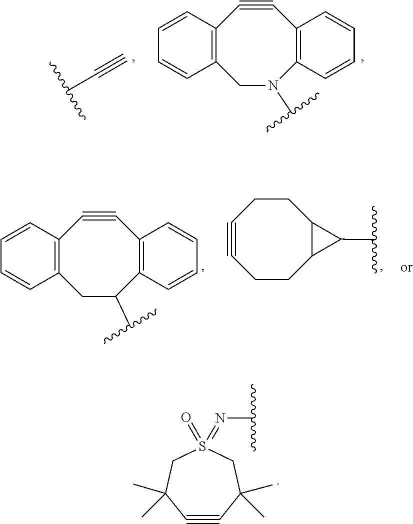

In some embodiments, Z is

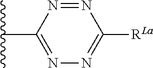

and the first member of a reactive pair comprises

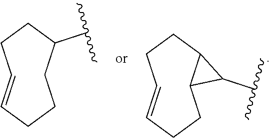 [SMILES: *C1CC/C=C/CCC1 ; *C1C2CC/C=C/CCC12]

In some embodiments, Z is —SH and first member of a reactive pair is

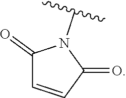 [SMILES: *N1C(=O)C=CC1=O]

In some embodiments, Z is

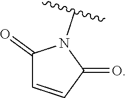 [SMILES: *N1C(=O)C=CC1=O]

and first member of a reactive pair is —SH.

In some embodiments, Z is

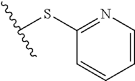 [SMILES: *Sc1ccccn1]

and first member of a reactive pair is —SH.

In some embodiments, Z is —SH and first member of a reactive pair is

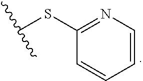 [SMILES: *Sc1ccccn1]

In some embodiments, Z comprises —CH═CH₂ and first member of a reactive pair comprises —CH═CH₂, and the contacting is in the presence of an olefin metathesis catalyst (e.g., Grubbs' catalyst, Benzylidene-bis(tricyclohexylphosphino)-dichlororuthenium).

In some embodiments, Z is —N═C═O or —N═C═S and first member of a reactive pair is H₂N—HN(R)—, HO—, HS—.

In some embodiments, wherein R¹ is C₁₋₆alkyl, the process further comprises contacting the oligonucleotide conjugate with a reagent capable of converting the —COOR¹ group to a —COOH group, or a salt thereof. In some embodiments, the reagent comprises a base; e.g., the base is a secondary amine, such as piperidine.

In some embodiments, wherein R¹ is C₁₋₆alkyl, the first member of a reactive pair is an azide, a terminal alkyne, a cycloalkyne, a trans-cycloalkene, or a tetrazine group.

In some embodiments, wherein R¹ is C₁₋₆alkyl, the first member of a reactive pair is an azide, and the second member of the reactive pair is a terminal alkyne, cycloalkyne, trans-cycloalkene, or tetrazine group.

In some embodiments, wherein R¹ is C₁₋₆alkyl, the first member of a reactive pair is a terminal alkyne, cycloalkyne, trans-cycloalkene, or tetrazine group, and the second member of the reactive pair is an azide.

In some embodiments, the oligonucleotide is of the Formula (XX):

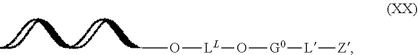

wherein
Z′ is a member of a reactive pair;
G⁰, L′, and L^{L} are each as defined for Formula (X); wherein L^{L} connects to a a terminal or internal position on the oligonucleotide.

In some embodiments, the oligonucleotide is of the Formula (XX-3′), (XX-5′), or (XX-2′):

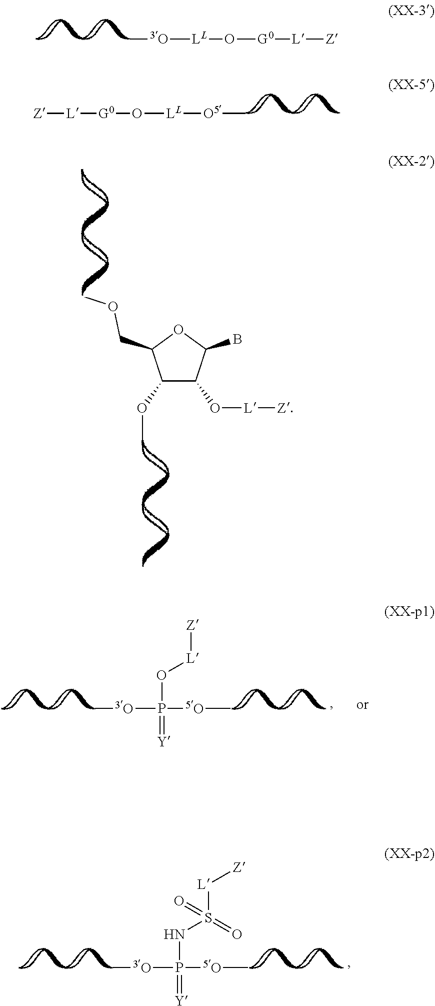

wherein Y is O or S (e.g., O); Z′ is a member of a reactive pair; L^{L} is —P(O)(OH)— or —P(S)(OH)—; G⁰ and L′ are as defined in any Formula or embodiment above, or a salt thereof.

In Formula (XX-2′), in one embodiment, the internal nucleoside of Formula (XX-2′) can be represented by one of,

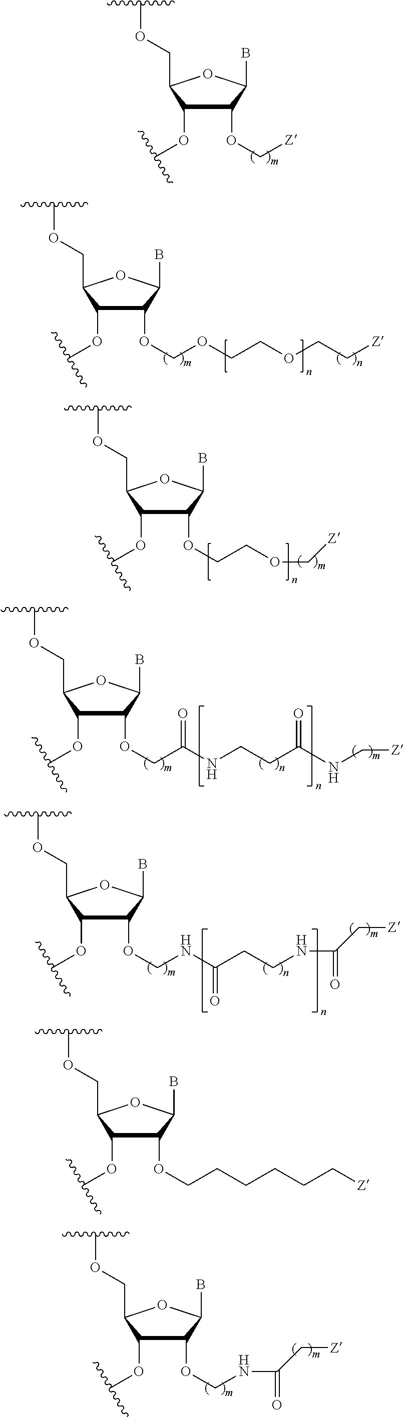

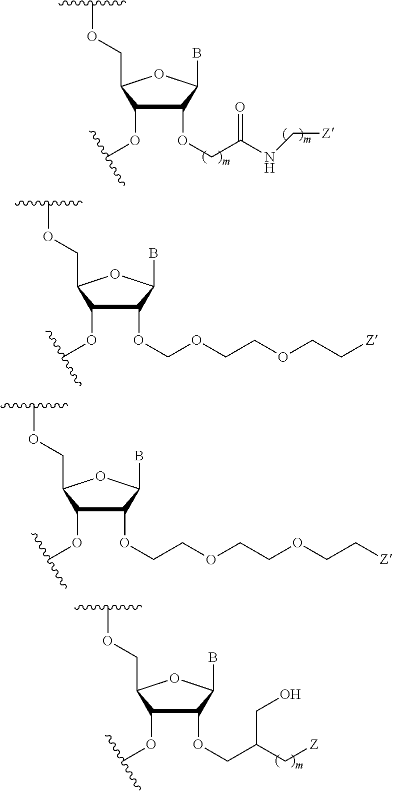

wherein
B is an optionally modified nucleobase (e.g., adenine, cytosine, uracil, guanine, 5-methylcytosine, or -5-methyluracil);
each n is independently 0 or an integer selected from 1-10; (e.g., 1-5, or 1-3, or 3, or 2, or 1);
each m n is independently integer selected from 1-20 (e.g., 2-12, or 2-10; or 2-6; or 2; or 3; or 4; or 5; or 6).

In Formula (XX-2′), in one embodiment, the internal nucleoside of Formula (XX-2′) can be represented by one of,

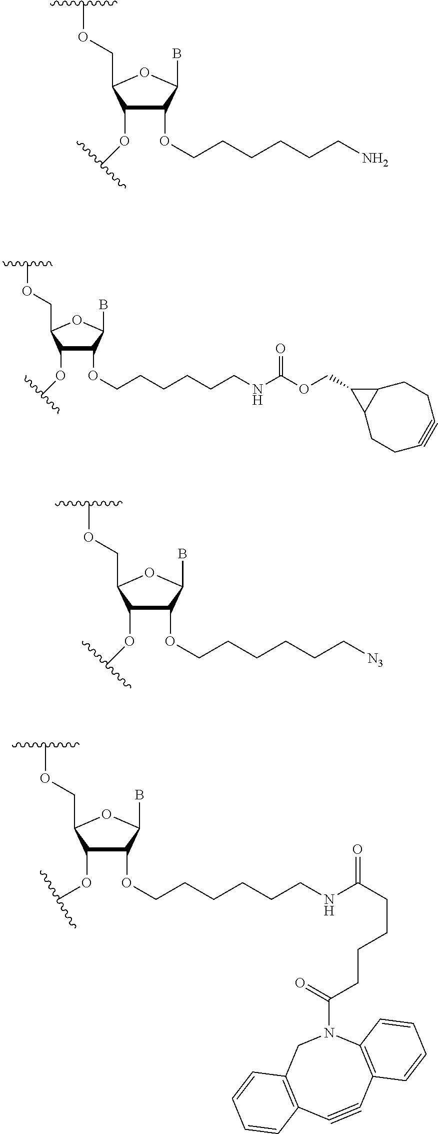

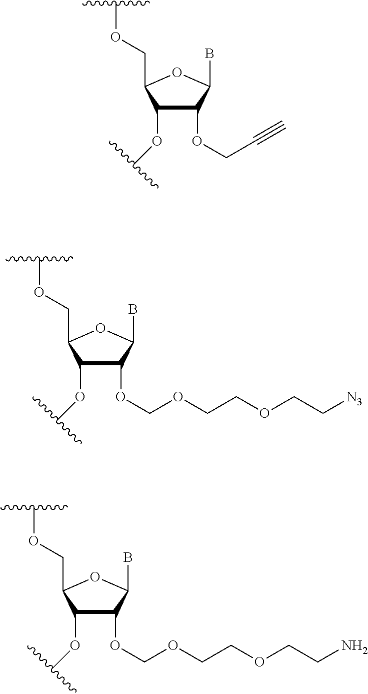

In some embodiments of Formula (XX-2′), two adjacent nucleosides in the oligonucleotide have one of the formula

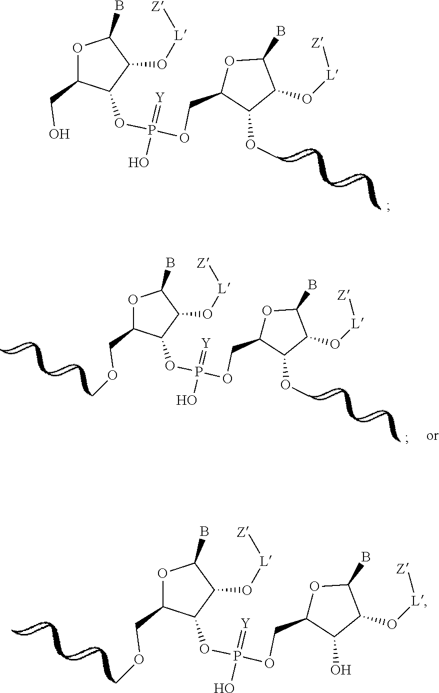

wherein Y is O or S (or O; or S); [symbol] represents the remainder for the oligonucleotide, and B is an optionally modified nucleobase.

In some embodiments of Formula (XX-2′), three adjacent nucleosides in the oligonucleotide have the formula

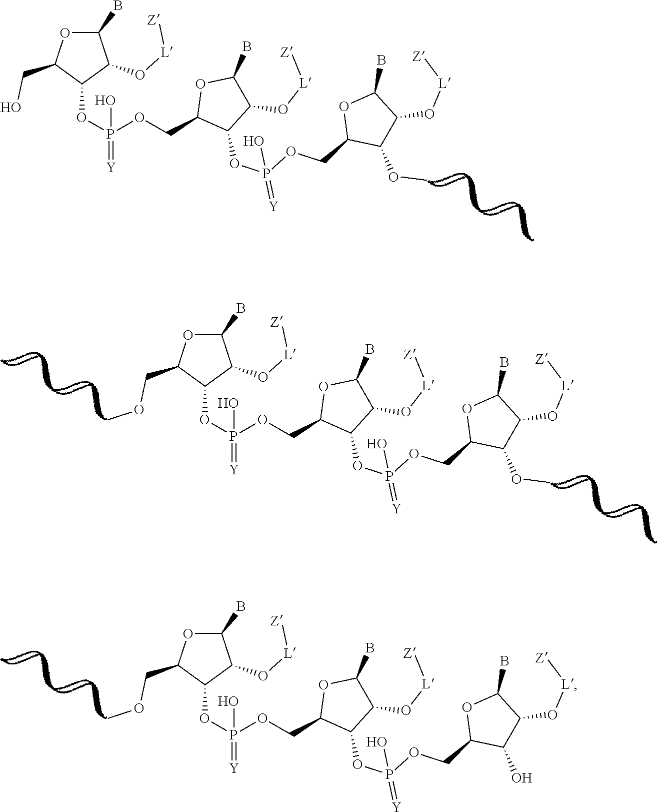

wherein each Y is independently O or S (or O; or S; or O then S or S then O, 5′ followed by 3′); [symbol] represents the remainder for the oligonucleotide, and B is an optionally modified nucleobase; e.g., each Y is O.
In some embodiments of Formula (XX-2′), four adjacent nucleosides in the oligonucleotide have the formula,

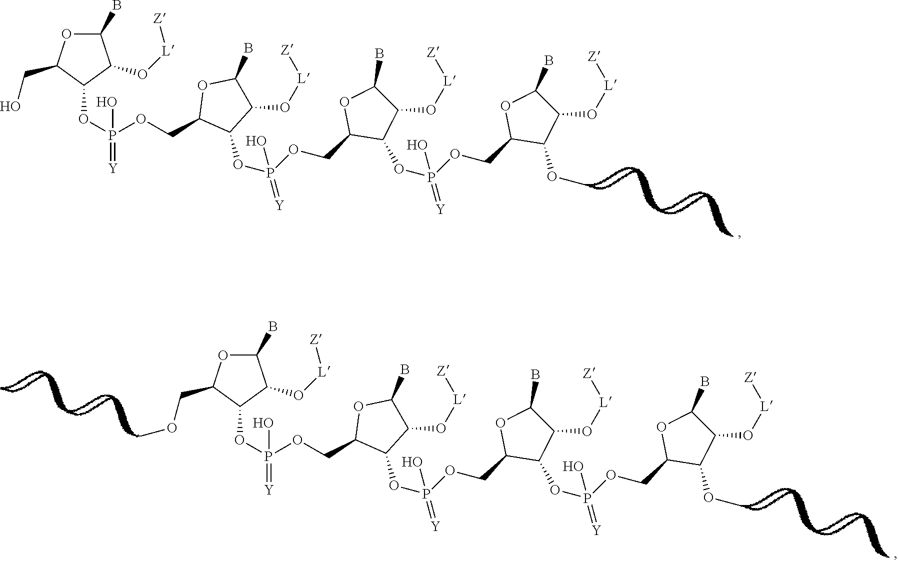

wherein each Y is independently O or S; [symbol] represents the remainder for the oligonucleotide, and B is an optionally modified nucleobase; e.g., each Y is O.

Examples of (XX-p1) and (XX-p2) include,

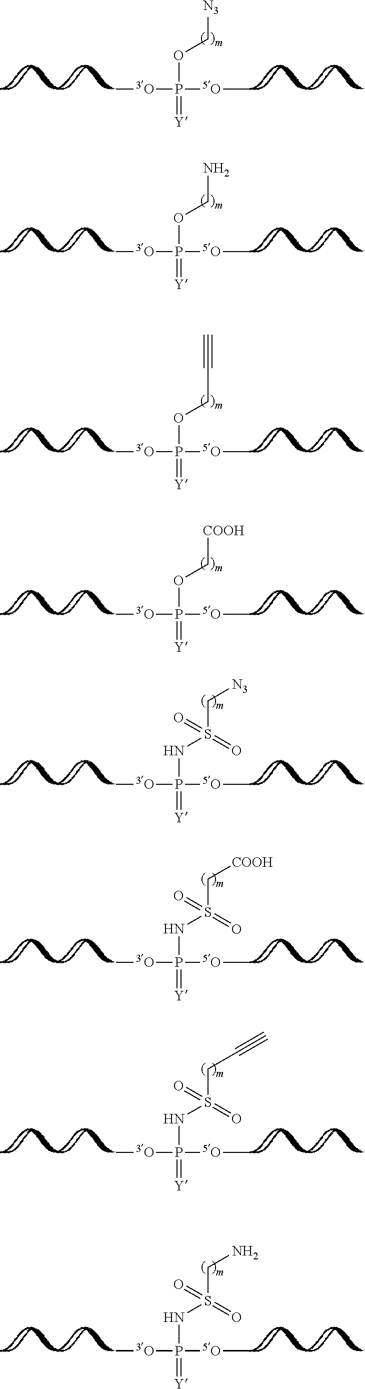

In Formula (XX-5′), in one embodiment, the terminal nucleoside can be represented by,

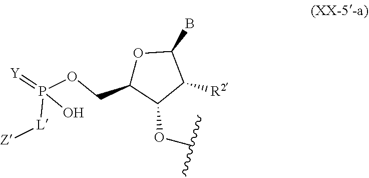

wherein B is an optionally modified nucleobase; wherein Y is O or S (e.g., S); and R² is hydrogen, halogen (e.g., fluoro), hydroxy, C₁₋₆alkoxy, C₁₋₆alkoxyC₁₋₆alkoxy (e.g., 2-methoxyethoxy), 2-(N-methylamino)-2-oxoethoxy.

In one embodiment of Formula (XX-5′-a), R^{2′} is methoxy. In another embodiment, B is uracil or 5-methyluracil. In another embodiment, B is uracil or 5-methyluracil and R^{2′} is methoxy. In another embodiment, B is adenine. In another embodiment, B is adenine and R^{2′} is methoxy. In another embodiment, B is cytosine or 5-methylcytosine. In another embodiment, B is cytosine or 5-methylcytosine and R^{2′} is methoxy. In another embodiment, B is guanine. In another embodiment, B is guanine and R^{2′} is methoxy.

In another embodiment of Formula (XX-5′-a), R^{2′} is fluoro. In another embodiment, B is uracil or 5-methyluracil. In another embodiment, B is uracil or 5-methyluracil and R^{2′} is fluoro. In another embodiment, B is adenine. In another embodiment, B is adenine and R^{2′} is fluoro. In another embodiment, B is cytosine or 5-methylcytosine. In another embodiment, B is cytosine or 5-methylcytosine and R^{2′} is fluoro. In another embodiment, B is guanine. In another embodiment, B is guanine and R^{2′} is fluoro.

In Formula (XX-3′), in one embodiment, the terminal nucleoside can be represented by,

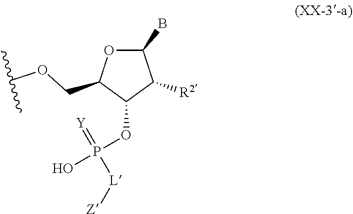

wherein B is an optionally modified nucleobase; wherein Y is O or S (e.g., S); and R^{2′} is hydrogen, halogen (e.g., fluoro), hydroxy, C₁₋₆alkoxy, C₁._alkoxyC₁₋₆alkoxy (e.g., 2-methoxyethoxy), 2-(N-methylamino)-2-oxoethoxy.

In one embodiment of Formula (XX-3′-a), R^{2′} is methoxy. In another embodiment, B is uracil or 5-methyluracil. In another embodiment, B is uracil or 5-methyluracil and R^{2′} is methoxy. In another embodiment, B is adenine. In another embodiment, B is adenine and R^{2′} is methoxy. In another embodiment, B is cytosine or 5-methylcytosine. In another embodiment, B is cytosine or 5-methylcytosine and R^{2′} is methoxy. In another embodiment, B is guanine. In another embodiment, B is guanine and R^{2′} is methoxy.

In another embodiment of Formula (XX-3′-a), R^{2′} is fluoro. In another embodiment, B is uracil or 5-methyluracil. In another embodiment, B is uracil or 5-methyluracil and R^{2′} is fluoro. In another embodiment, B is adenine. In another embodiment, B is adenine and R^{2′} is fluoro. In another embodiment, B is cytosine or 5-methylcytosine. In another embodiment, B is cytosine or 5-methylcytosine and R^{2′} is fluoro. In another embodiment, B is guanine. In another embodiment, B is guanine and R^{2′} is fluoro.

In some embodiments of Formula (XX-3′), (XX-5′), and (XX-2′) and any preceding embodiment thereof, -L′- is -L′-[G-L²]-G-L³-*, wherein * is the bond to Z′.

In some embodiments, -L′- is -L′-G-L³-*, wherein * is the bond to Z′.

In some embodiments, -L′- is —C₂₋₃₀alkyl-*, wherein * is the bond to Z′.

In some embodiments, -L′- is -L¹-[G-L²]_{q}-G-L³-*, wherein * is the bond to Z′;
q is 0, 1, 2, 3, 4, or 5;
L¹ is a bond or -B-A-;
each L² is independently -A-B-A-;
L³ is a bond or -A-B-A-;
each A is independently a bond, —O—, —S—, or —N(R^{N})—;
each B is independently a bond, CH₂, C(O), C(S), C(NR^{N}), S(O), S(O)₂, P(O)(OH), P(S)(OH), or P(S)(SH);
each R^{N} is independently hydrogen or C₁₋₆alkyl; and
each G is independently C₁₋₁₀alkyl, C₂₋₁₀alkenyl, C₂₋₁₀alkynyl, each of which is optionally substituted with 1, 2, 3, or 4 R groups.

In some embodiments, -L′- is -L¹-[G-L²]_{q}-G-*, wherein * is the bond to Z′
q is 0, 1, 2, or 3;
L¹ is a bond or -B-A-;
L¹ is a bond or -B-A-;
each L² is independently a bond, C(O)O, OC(O), C(O)(NR^{N}), N(R^{N})C(O), SO₂N(R^{N}), N(R^{N})SO₂, OP(O)(OH), OP(S)(OH), P(O)(OH)O, P(S)(OH)O, OP(O)(OH)O, or OP(S)(OH)O, wherein each R^{N} is independently hydrogen or C₁₋₆alkyl; and
each G is independently C₁₋₁₀alkyl, C₂₋₁₀alkenyl, C₂₋₁₀alkynyl, each of which is optionally substituted with 1 or 2 R groups.

In some embodiments, -L′- is -L¹-[G-L²]_{q}-G-*, wherein * is the bond to Z′;
q is 0, 1, 2, or 3;
L¹ is a bond or -B-A-;
each L² is independently a bond, C(O)O, OC(O), C(O)(NR^{N}), N(R^{N})C(O), OP(O)(OH)O, or OP(S)(OH)O, wherein each R^{N} is independently hydrogen or C₁₋₆ alkyl; and
each G is independently C₁₋₁₀alkyl or C₂₋₁₀alkenyl, each of which is optionally substituted with 1 or 2 R groups.

In some embodiments, -L′- is -[G-L²]_{q}-G-*, wherein * is the bond to Z′;
q is 0, 1, 2, or 3 (e.g., q is 0, 1, or 2; or 0 or 1; or 0; or 1; or 2);
each L² is independently C(O)O or OC(O); and
each G is independently C₁₋₁₀alkyl, each of which is optionally substituted with 1 or 2 R groups.

In some embodiments, -L′- is -[G-L²]_{q}-G-*, wherein * is the bond to Z′;
q is 0, 1, 2, or 3 (e.g., q is 0, 1, or 2; or 0 or 1; or 0; or 1; or 2);
each L² is independently C(O)(NR^{N}) or N(R^{N})C(O), wherein each R^{N} is independently hydrogen or C₁₋₆alkyl; and
each G is independently C₁₋₁₀alkyl, each of which is optionally substituted with 1 or 2 R groups.

In some embodiments, -L′- is -[G-L²]_{q}-G-*, wherein * is the bond to Z′;
q is 0, 1, 2, or 3 (e.g., q is 0, 1, or 2; or 0 or 1; or 0; or 1; or 2);
each L² is independently OP(O)(OH)O, or OP(S)(OH)O (e.g., each is OP(O)(OH)O); and
each G is independently C₁₋₁₀alkyl, each of which is optionally substituted with 1 or 2 R groups.

In some embodiments, -L′- is -[G-L²]_{q}-G-*, wherein * is the bond to Z′;
q is 0, 1, 2, or 3 (e.g., q is 0, 1, or 2; or 0 or 1; or 0; or 1; or 2);
each L² is a bond; and
each G is independently C₁₋₁₀alkyl, each of which is optionally substituted with 1 or 2 R groups.

In some embodiments, -L′-Z′ is selected from the group consisting of:

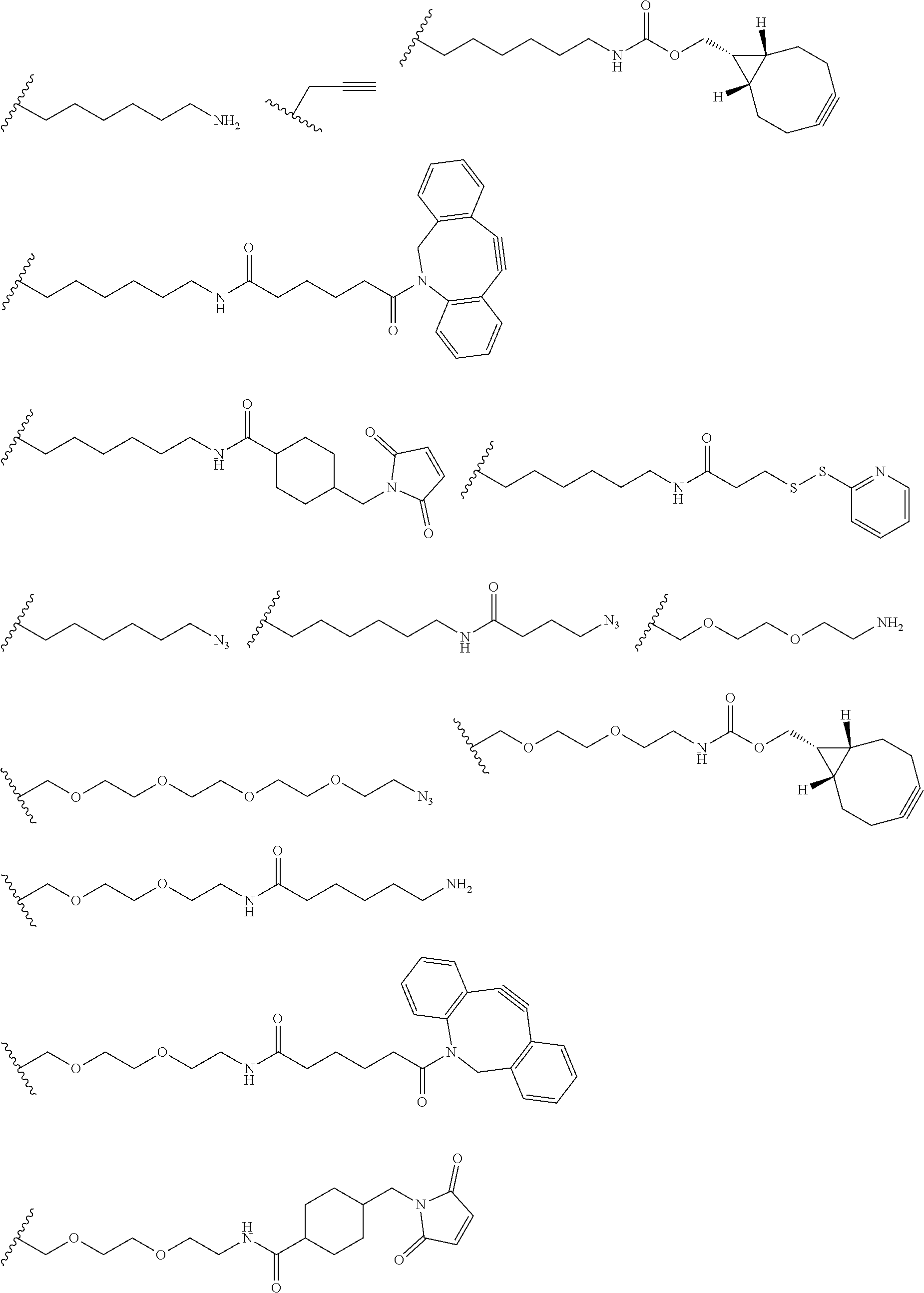

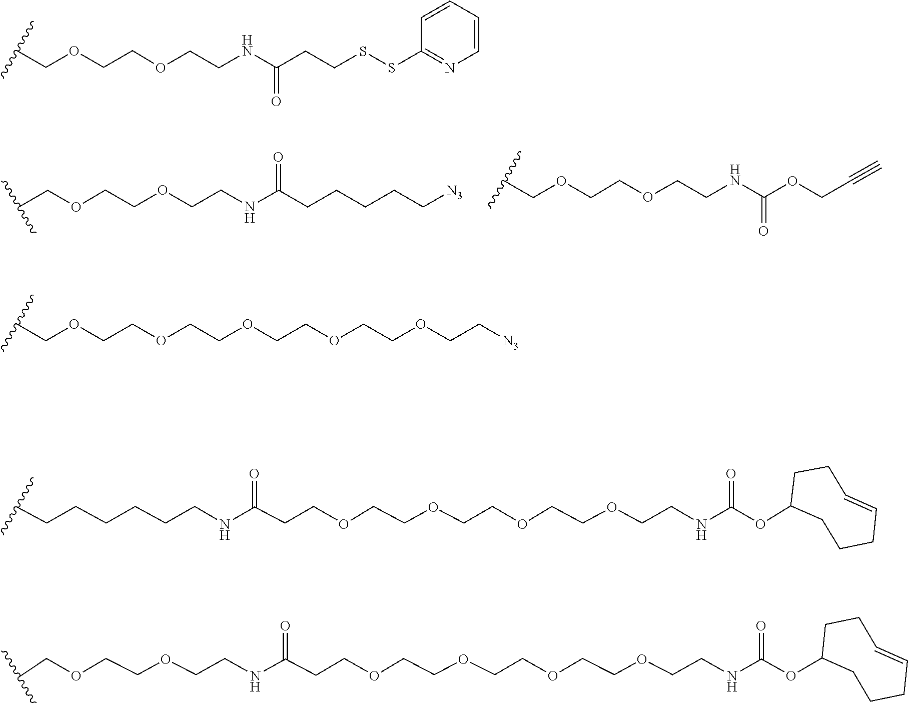 [SMILES: *COCCOCCNC(=O)CCSSc1ccccn1 ; *COCCOCCNC(=O)CCCCCN=[N+]=[N-] ; *COCCOCCNC(=O)OCC#C ; *COCCOCCOCCOCCOCCN=[N+]=[N-] ; *CCCCCCNC(=O)CCOCCOCCOCCOCCNC(=O)OC1CC/C=C/CCC1 ; *COCCOCCNC(=O)CCOCCOCCOCCOCCNC(=O)OC1CC/C=C/CCC1]

In some embodiments of Formula (XX-5′),

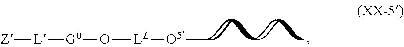

wherein O-G⁰-L′-Z′ is

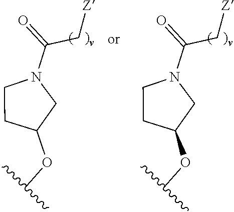

wherein v is an integer between 1 and 20 (e.g., an integer between 1 and 16, an integer between 1 and 12, an integer between 1 and 10, and integer between 3 and 9, 3, 4, 5, 6, 7, 8 or 9).

In other embodiments of Formula (XX-5′), —O-G⁰-L′-Z′ is selected from the group consisting of:

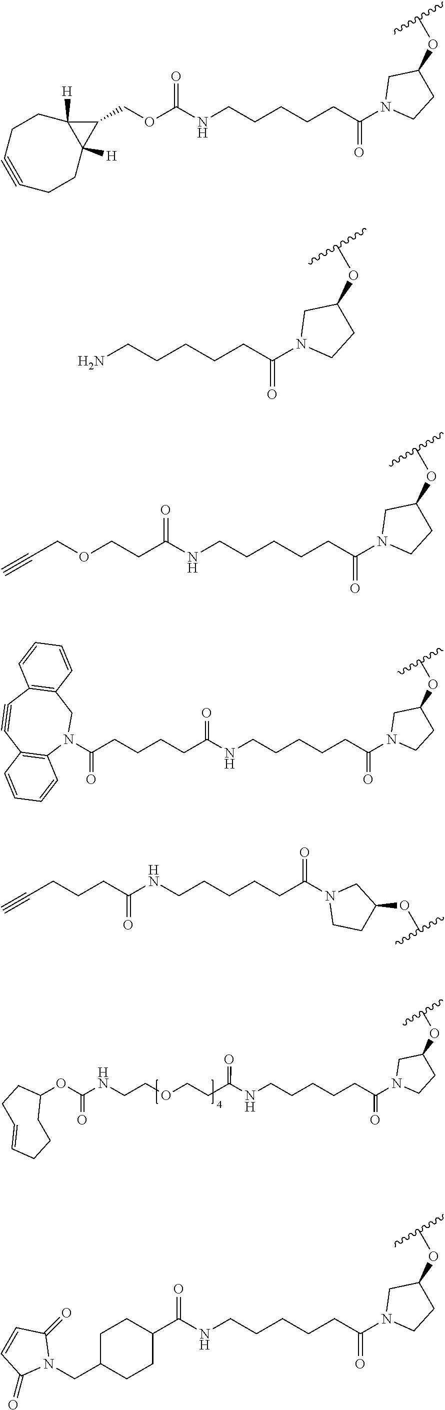

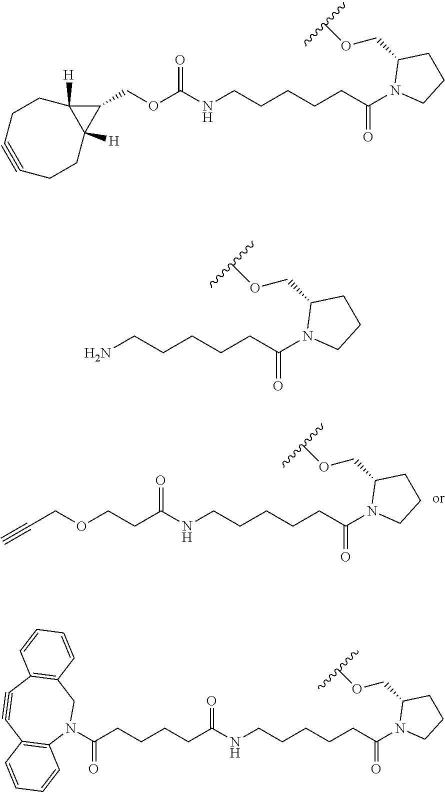 [SMILES: *OC[C@@H]1CCCN1C(=O)CCCCCNC(=O)OC[C@@H]1[C@@H]2CCC#CCC[C@@H]21 ; *OC[C@@H]1CCCN1C(=O)CCCCCN ; *OC[C@@H]1CCCN1C(=O)CCCCCNC(=O)CCOCC#C ; *OC[C@@H]1CCCN1C(=O)CCCCCNC(=O)CCCCC(=O)N1Cc2ccccc2C#Cc2ccccc21]

In some embodiments of Formula (XX-3′),

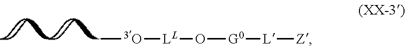

### O-G⁰ L′-Z′ is

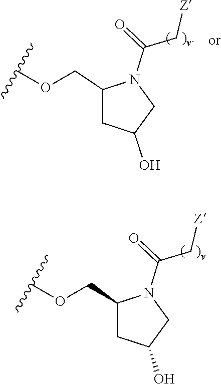

or OH wherein v is an integer between 1 and 20, (e.g., an integer between 1 and 16, an integer between 1 and 12, an integer between 1 and 10, and integer between 3 and 9, 3, 4, 5, 6, 7, 8 or 9).

In other embodiments of Formula (XX-3′), —O-G⁰-L′-Z′ is selected from the group consisting of:

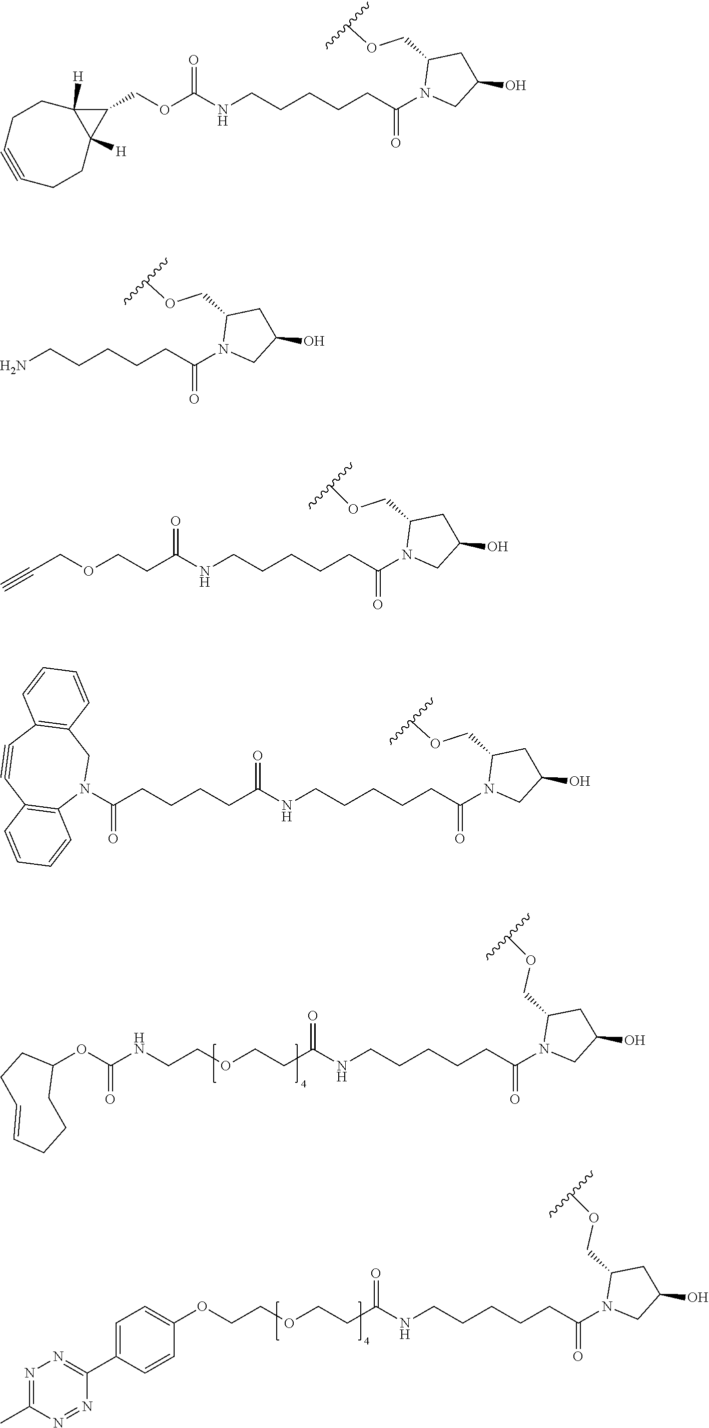

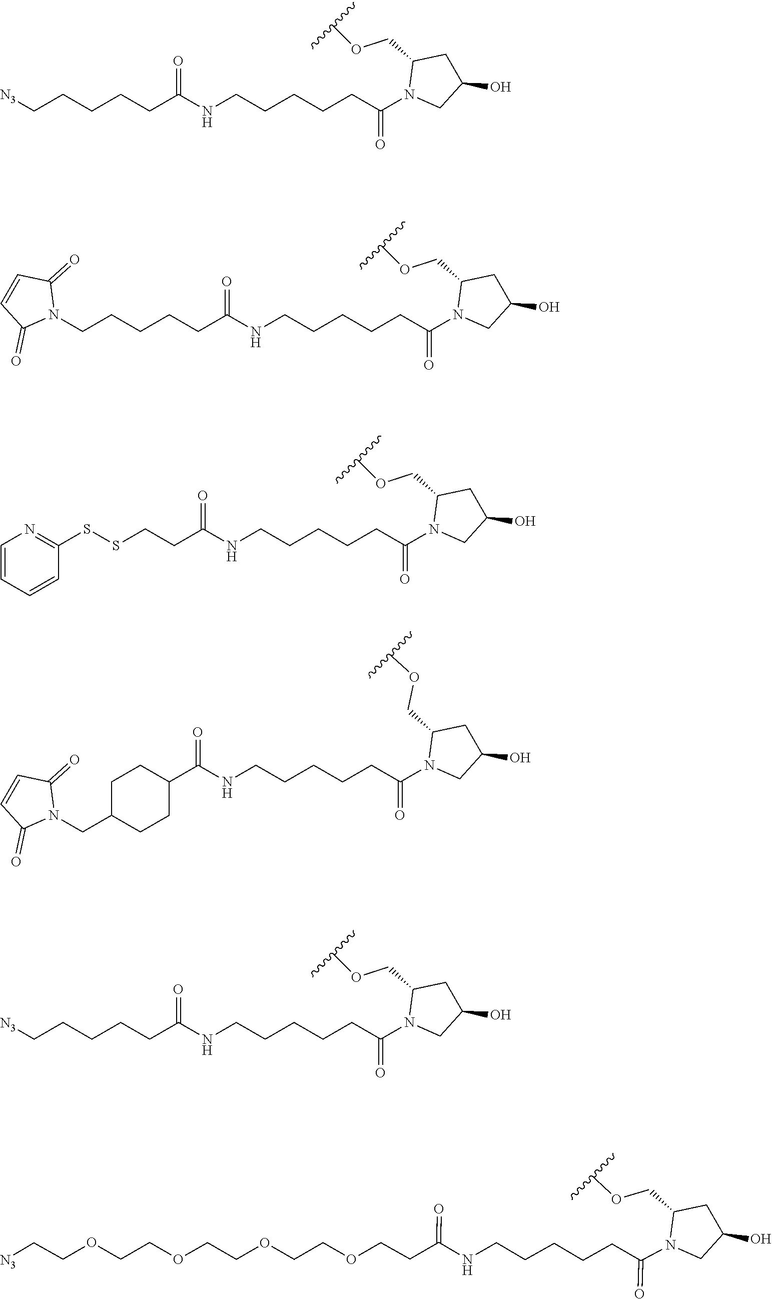 [SMILES: *OC[C@@H]1C[C@@H](O)CN1C(=O)CCCCCNC(=O)CCCCCN=[N+]=[N-] ; *OC[C@@H]1C[C@@H](O)CN1C(=O)CCCCCNC(=O)CCCCCN1C(=O)C=CC1=O ; *OC[C@@H]1C[C@@H](O)CN1C(=O)CCCCCNC(=O)CCSSc1ccccn1 ; *OC[C@@H]1C[C@@H](O)CN1C(=O)CCCCCNC(=O)C1CCC(CN2C(=O)C=CC2=O)CC1 ; *OC[C@@H]1C[C@@H](O)CN1C(=O)CCCCCNC(=O)CCCCCN=[N+]=[N-] ; *OC[C@@H]1C[C@@H](O)CN1C(=O)CCCCCNC(=O)CCOCCOCCOCCOCCN=[N+]=[N-]]

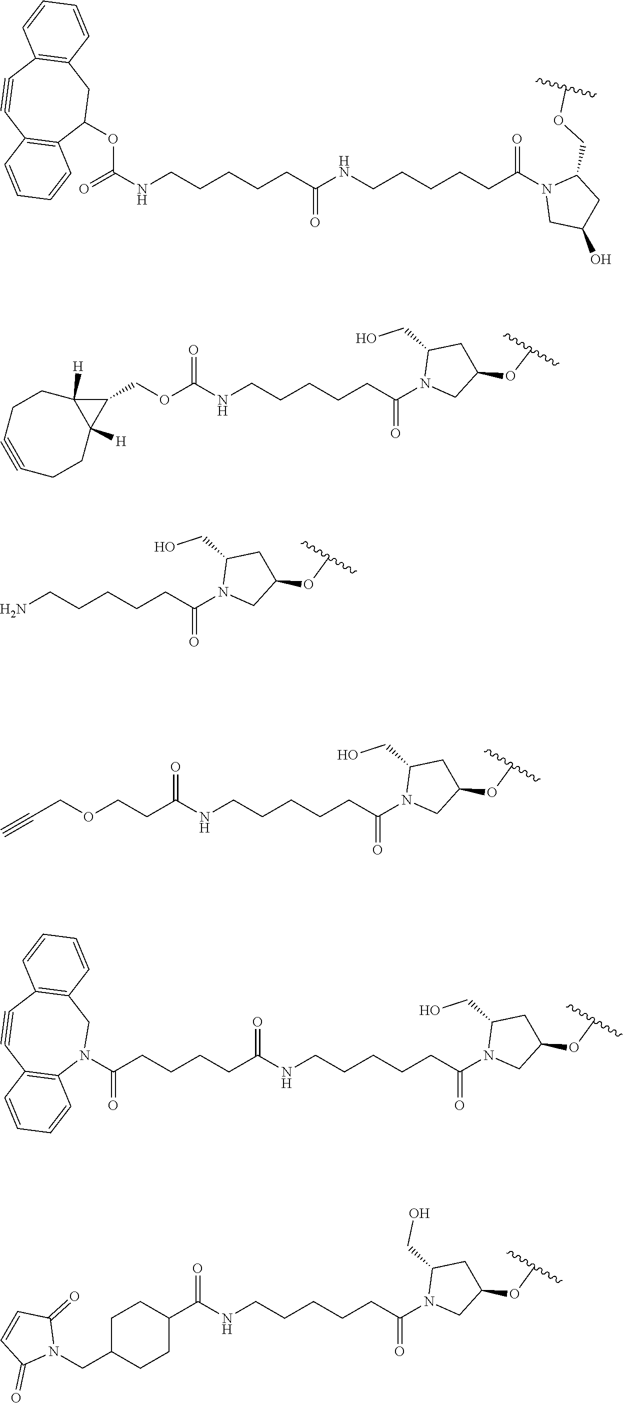 [SMILES: *OC[C@@H]1C[C@@H](O)CN1C(=O)CCCCCNC(=O)CCCCCNC(=O)OC1Cc2ccccc2C#Cc2ccccc21 ; *O[C@@H]1C[C@@H](CO)N(C(=O)CCCCCNC(=O)OC[C@@H]2[C@@H]3CCC#CCC[C@@H]32)C1 ; *O[C@@H]1C[C@@H](CO)N(C(=O)CCCCCN)C1 ; *O[C@@H]1C[C@@H](CO)N(C(=O)CCCCCNC(=O)CCOCC#C)C1 ; *O[C@@H]1C[C@@H](CO)N(C(=O)CCCCCNC(=O)CCCCC(=O)N2Cc3ccccc3C#Cc3ccccc32)C1 ; *O[C@@H]1C[C@@H](CO)N(C(=O)CCCCCNC(=O)C2CCC(CN3C(=O)C=CC3=O)CC2)C1]

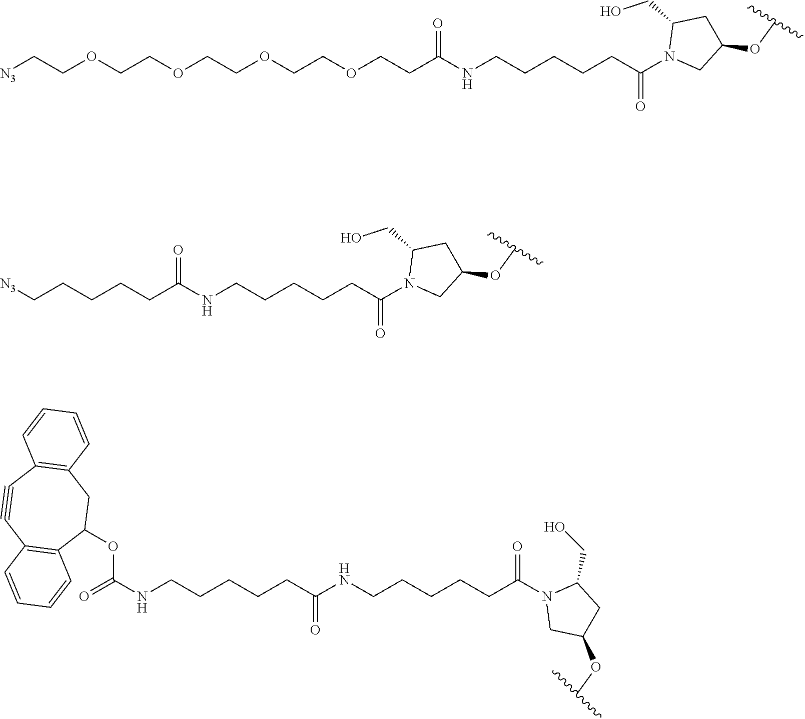 [SMILES: *O[C@@H]1C[C@@H](CO)N(C(=O)CCCCCNC(=O)CCOCCOCCOCCOCCN=[N+]=[N-])C1 ; *O[C@@H]1C[C@@H](CO)N(C(=O)CCCCCNC(=O)CCCCCN=[N+]=[N-])C1 ; *O[C@@H]1C[C@@H](CO)N(C(=O)CCCCCNC(=O)CCCCCNC(=O)OC2Cc3ccccc3C#Cc3ccccc32)C1]

The process described above, using a compound of Formula (IV), where Z is a member of a reactive pair, with an oligonucleotide of Formula (XX), (XX-2′), (XX-3′) or (XX-5′) results in generating an oligonucleotide of the formula,

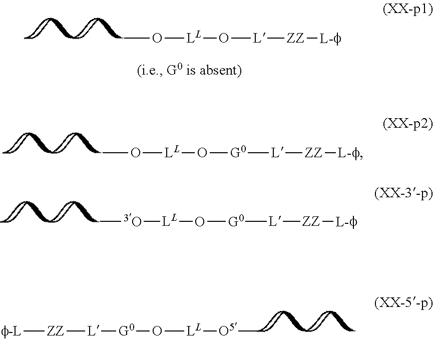

wherein ZZ is formed by reaction of the reactive pair represented by Z and Z′, respectively.
-L-Φ is according Formula (XII) and any embodiment thereof;
ZZ is a covalent construct formed by the reactive pair (i.e., Z and Z′);
L′, G⁰, and L^{L} are as defined in Formula (X), including any embodiment thereof. In certain embodiments, LL is —P(O)(OH)—. In other embodiments, L^{L} is —P(S)(OH)—.

The process described above, using a compound of Formula (V), where Z⁰ is a member of a reactive pair, with an oligonucleotide of Formula (XX), (XX-2′), (XX-3′) or (XX-5′) results in generating an oligonucleotide of the formula,

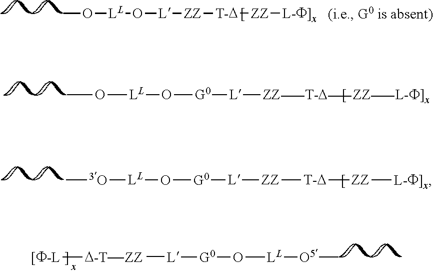

wherein ZZ is formed by reaction of the reactive pair represented by Z⁰ and Z′, respectively.
-L-Φ is according Formula (XII) and any embodiment thereof;
ZZ is a covalent construct formed by the reactive pair (i.e., Z⁰ and Z′);
L′, G⁰, and L^{L} are as defined in Formula (XV), including any embodiment thereof. In certain embodiments, LL is —P(O)(OH)—. In other embodiments, L^{L} is —P(S)(OH)—.

In certain embodiments, the process described can utilize an oligonucleotide of the formula,

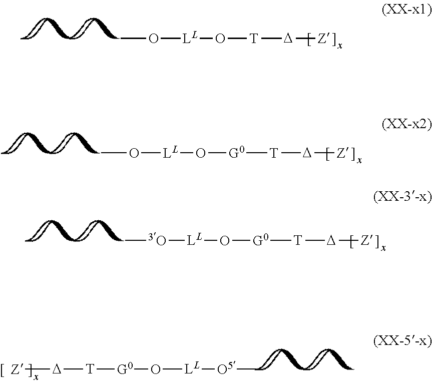

with a compound of Formula (IV), where Z is a member of a reactive pair, results, respectively, in generating an oligonucleotide of the formula,

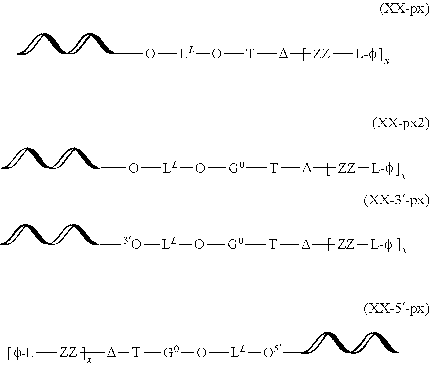

wherein
-L-Φ is according Formula (XII) and any embodiment thereof;
ZZ is a covalent construct formed by the reactive pair (i.e., Z and Z′); and
T, G⁰, and L^{L} are as defined in Formula (X), including any embodiment thereof. In certain embodiments, LL is —P(O)(OH)—. In other embodiments, L^{L} is —P(S)(OH)—.

In certain embodiments, the process described can utilize an oligonucleotide of the Formula (XX-x1), (XX-x2), (XX-3′-x), or (XX-5′-x), with a compound of Formula (V), where Z⁰ is a member of a reactive pair, results, respectively, in generating an oligonucleotide of the formula,

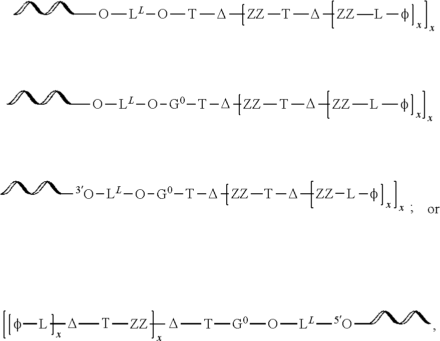

respectively, wherein
-L-Φ is according Formula (XII) and any embodiment thereof;
ZZ is a covalent construct formed by the reactive pair (i.e., Z and Z′ or Z⁰ and Z′); and
T, G⁰, and LL are as defined in Formula (XV), including any embodiment thereof. In certain embodiments, LL is —P(O)(OH)—. In other embodiments, L^{L} is —P(S)(OH)—.

For example, in oligonucleotides of any of the preceding formulae containing Δ, can be provided where Δ is of the formula,

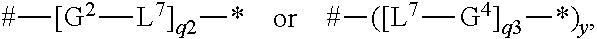

wherein
# is the bond to T;
y is 1, 2, 3, 4, or 5;
q2 is 1, 2, 3, 4, 5, 6, 7, or 8;
q3 is 0, 1, 2, 3, 4, 5, 6, 7, or 8;
each G², G³, and G⁴ is independently -D²-E²-F²-, wherein
   D², E², and F² are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, 4, or 5 R groups, and
   wherein each G² and G⁴ optionally contains at least one bond to a Z′ (e.g., one bond to a Z′);
or G³ is N and y is 2;
each L⁷ is independently -A²-B²-A²-; wherein
   each A² is independently a bond, —O—, —S—, or —N(R^{N2})—;
   each B² is independently a bond, C(O), C(S), C(NR^{N2}), S(O), S(O)₂, P(O)(OH), P(S)(OH), or P(S)(SH); and
   each R^{N2} is independently hydrogen, C₁₋₆alkyl, a bond to a Z′, or two R^{N2} within an -A²-B²-A²- group taken together with the atoms to which they are connected from a 4-8 membered heterocyclyl;
and
each R^{B} is independently halogen, cyano, azido, nitro, —N(R¹⁰)₂, —O(R¹⁰), —S(R¹⁰), —C(O)OR¹⁰, —C(O)R¹⁰, —C(O)N(R¹⁰)₂, —C(NR¹⁰)OR¹⁰, —C(NR¹⁰)R¹⁰, —C(NR¹⁰)N(R¹⁰)₂, —C(S)OR¹⁰, —C(S)R¹⁰, —C(S)N(R¹⁰)₂, —S(O)₂R¹⁰, —S(O)₂OR¹⁰, —S(O)₂N(R¹⁰)₂, —N(R¹⁰)C(O)OR¹⁰, —N(R¹⁰)C(O) R¹⁰, —N(R¹⁰)C(O)N(R¹⁰)₂, —N(R¹⁰)S(O)₂R¹⁰, —N(R¹⁰)S(O)₂OR¹⁰, —N(R¹⁰)S(O)₂N(R¹⁰)₂, —OC(O)OR¹⁰, —OC(O)R¹⁰, —OC(O)N(R¹⁰)₂, —OS(O)₂R¹⁰, —OS(O)₂OR¹⁰, —OS(O)₂N(R¹⁰)₂, or —SC(O)R¹⁰, wherein each R¹⁰ is independently hydrogen, C₁₋₁₀alkyl, C₂₋₁₀alkenyl, C₂₋₁₀alkynyl, C₃₋₁₀cycloalkyl, 3-10 membered heterocyclyl, aryl, or heteroaryl. provided that Δ contains x bonds to Z′.

In one embodiment, -Δ- is #-[G²-L⁷]_{q2}-*, where # is the bond to T and * is a bond to a Z′ group. For example, such embodiments include the following,

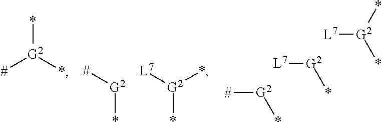

wherein each * is a bond to a Z′; # is the bond to T,
each G² is independently C₁₋₁₀alkyl, C₂₋₁₀alkenyl, C₃₋₁₀cycloalkyl, 3-10 membered heterocyclyl, aryl, or heteroaryl, each of which is optionally substituted with 1, 2, 3, 4, or 5 R^{B} groups; and each L⁷ is independently -A²-B²-A²-, wherein each A² is independently a bond, —O—, —S—, or —N(R^{N2})—; each B² is independently a bond, C(O), S(O)₂, P(O)(OH), or P(S)(OH).

In one embodiment, each G² is independently C₁₋₁₀alkyl, each optionally substituted with 1 or 2 R^{B} groups. In one embodiment, each G² is independently C₁₋₁₀alkyl.

In one embodiment, each L⁷ is independently -A²-B²-A²-, wherein each A² is independently a bond, —O—, —S—, or —N(R^{N2})—; and each B² is independently a bond, C(O) or S(O)₂, provided that at least one A² is not a bond.

In one embodiment, each L⁷ is independently -A²-B²— or -B²-A²-, wherein each A² is independently, —O—, —S—, or —N(R^{N2})—; and each B² is independently a bond, C(O) or S(O)₂.

For example, such embodiments include each of the following,

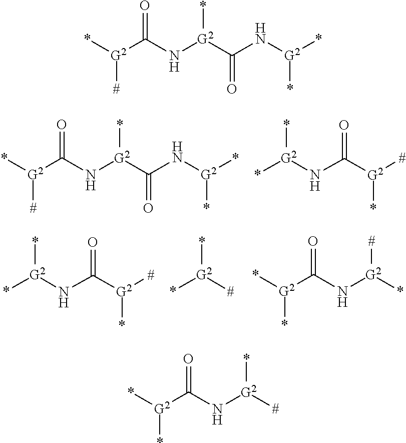

wherein # is the bond to T and each * is a bond to a Z′ group; and each G² is independently C₁₋₁₀alkyl.

For example, such embodiments include each of the following, wherein # is the bond to T and each * is a bond to a Z′;

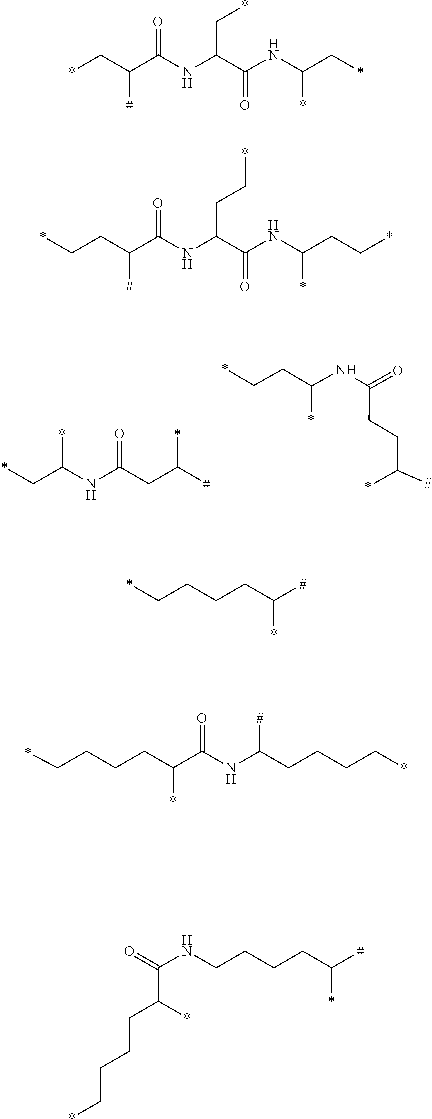

wherein # is the bond to T and each * is a bond to a Z′ group.

For example, such embodiments include each of the following,

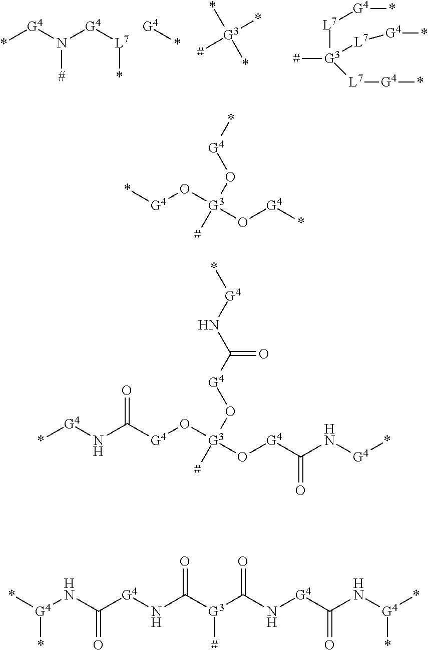

wherein # is the bond to T, each * is a bond to a Z′ group, and
each L⁷ is selected from the group consisting of —O—, —S—, —N(H)—, —C(O)O—, —OC(O)—, —C(O)N(H)—, —OC(O)O—, —N(H)C(O)O—, —OC(O)N(H)—, —OP(O)(OH)O—, or —OP(S)(OH)O—; and each G⁴ is independently -D²-E²-F²-, wherein
   each D² and F² are independently a bond or C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, 4, or 5 R groups, and
   each E² is independently bond, C₁₋₁₀alkyl, C₂₋₁₀alkenyl, C₃₋₁₀cycloalkyl, 3-10 membered heterocyclyl, aryl, or heteroaryl, each of which is optionally substituted with 1, 2, 3, 4, or 5 R^{B} groups, provided that E² is not a bond with D² and F² are each bonds.

For example, such embodiments include each of the following, wherein each * is a bond to a Z′;

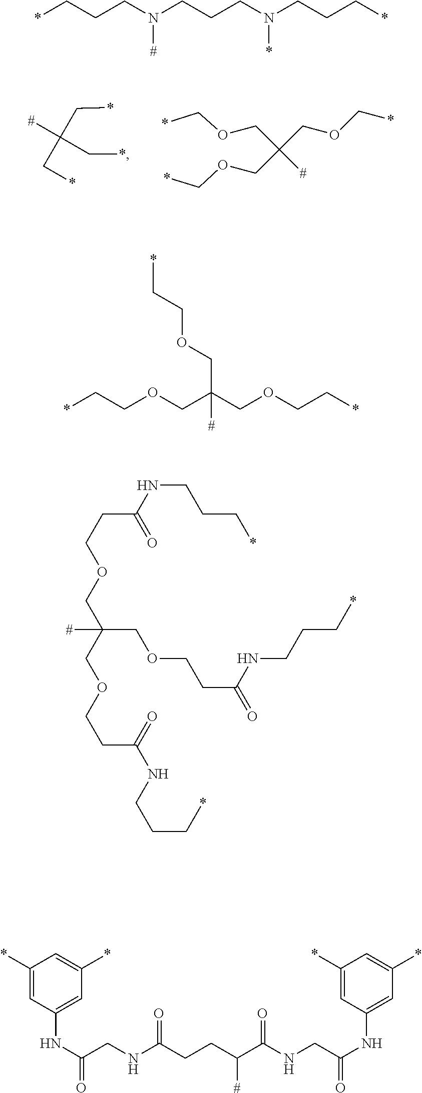

wherein # is the bond to T and each * is a bond to a Z′ group.

Exemplary reactive pairs that may be used in the context of the present disclosure are shown below, wherein, in each instance:
each R is independently C₁₋₁₀alkyl (e.g., methyl, ethyl, propyl, isopropyl, t-butyl, isobutyl, butyl, or hexyl);
R^{E} is forms an activated ester, as defined by any embodiment herein, or is R;
X is a leaving group (e.g., bromo, iodo, tosylate, mesylate, or triflate);
R^{La} is hydrogen, C₁₋₁₀alkyl (e.g., methyl, ethyl, propyl, isopropyl, t-butyl, isobutyl, butyl, or hexyl), C₃₋₈cycloalkyl, 3-8 membered heterocyclyl, aryl (e.g., phenyl), or heteroaryl (e.g., 2-pyridyl).

| Reactive Pairs (Z) and (Z′) throughout | | Covalent linkage formed by the reactive pair | |
|---|---|---|---|
| First member | Second member | (ZZ) | |
| —N₃ | 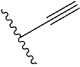 [SMILES: *C#C] | 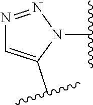 [SMILES: *c1cnnn1*] | |
| | | or | |
| | | 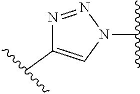 [SMILES: *c1cn(*)nn1] | |
| —N₃ | 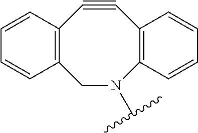 [SMILES: *N1Cc2ccccc2C#Cc2ccccc21] | 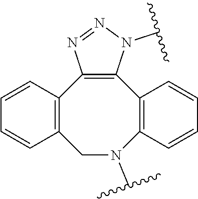 [SMILES: *N1Cc2ccccc2-c2nnn(*)c2-c2ccccc21] | |
| | | or | |
| | | 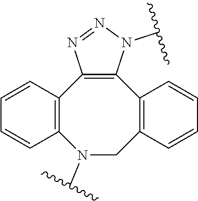 [SMILES: *N1Cc2ccccc2-c2c(nnn2*)-c2ccccc21] | |
| —N₃ | 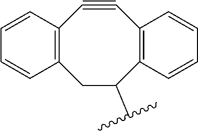 [SMILES: *C1Cc2ccccc2C#Cc2ccccc21] | 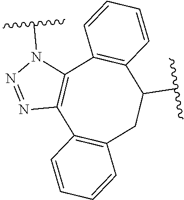 [SMILES: *C1Cc2ccccc2-c2nnn(*)c2-c2ccccc21] | |
| —N₃ | 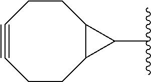 [SMILES: *C1C2CCC#CCCC12] | 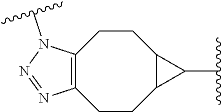 [SMILES: *C1C2CCc3nnn(*)c3CCC12] | |
| —N₃ | 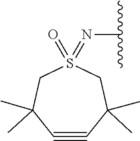 | 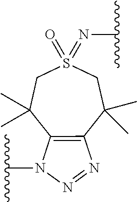 | |
| | | or | |
| | | 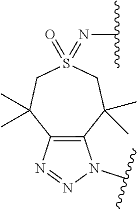 | |
| —N₃ | 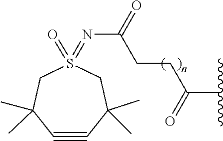 | 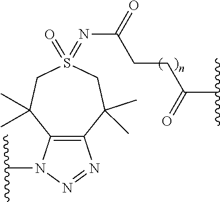 | |
| | | or | |
| | | 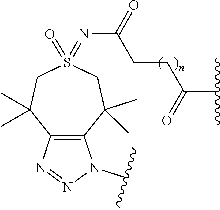 | n |

| | | |
|---|---|---|
| | | is 0-20, e.g., n is 1-11 |
| 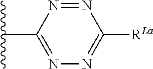 | 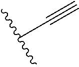 [SMILES: *C#C] | 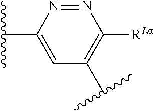 |
| | | or |
| | | 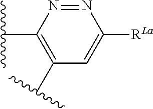 |
| 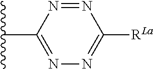 | 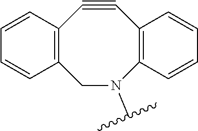 [SMILES: *N1Cc2ccccc2C#Cc2ccccc21] | 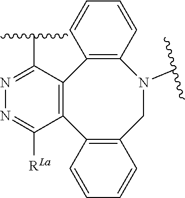 |
| | | or |
| | | 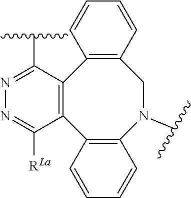 |
| 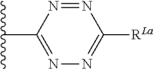 | 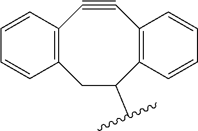 [SMILES: *C1Cc2ccccc2C#Cc2ccccc21] | 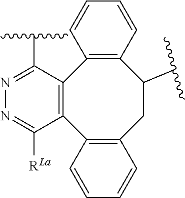 |
| 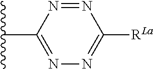 | 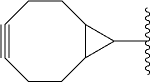 [SMILES: *C1C2CCC#CCCC12] | 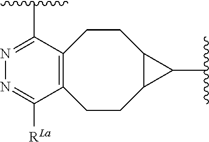 |
| 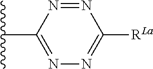 | 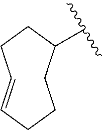 [SMILES: *C1CC/C=C/CCC1] | 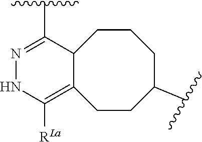 |
| | | or |
| | | 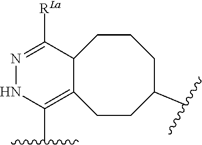 |
| 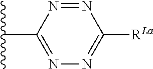 | 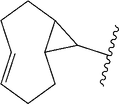 [SMILES: *C1C2CC/C=C/CCC12] | 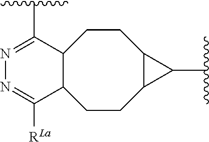 |
| —SH | 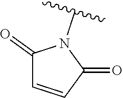 [SMILES: *N1C(=O)C=CC1=O] | 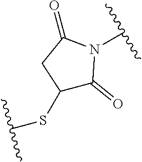 [SMILES: *SC1CC(=O)N(*)C1=O] |
| —C(H)═O | H₂N— | —C(H)═N— |
| —C(H)═O | H₂N—N(H)— | —C(H)═N—N(H)— |
| —C(H)═O | H₂N—N(H)C(O)— | —C(H)═N—N(H)C(O)— |
| —C(H)═O | H₂N—N(R)— | —C(H)═N—N(R)— |
| —C(H)═O | H₂N—N(R)C(O)— | —C(H)═N—N(R)C(O)— |
| —C(H)═O | H₂NO— | —C(H)═N—O— |
| —C(O)OH | H₂N— | —C(O)N(H)— |
| —C(O)OH | H₂N—N(H)— | —C(O)N(H)—N(H)— |
| —C(O)OH | H₂N—N(R)— | —C(O)N(H)—N(R)— |
| —C(O)OH | HN(R)— | —C(O)N(R)— |
| —C(O)OH | HO— | —C(O)O— |
| —C(O)OH | HS— | —C(O)S— |
| —C(O)OR^{E} | H₂N— | —C(O)N(H)— |
| —C(O)OR^{E} | H₂N—N(H)— | —C(O)N(H)—N(H)— |
| —C(O)OR^{E} | H₂N—N(R)— | —C(O)N(H)—N(R)— |
| —C(O)OR^{E} | HN(R)— | —C(O)N(R)— |
| —C(S)OH | H₂N— | —C(S)N(H)— |
| —C(S)OH | HN(R)— | —C(S)N(R)— |
| —C(S)OH | HO— | —C(S)O— |
| —C(S)OH | HS— | —C(S)S— |
| —C(S)OR^{E} | H₂N— | —C(S)N(H)— |
| —C(S)OR^{E} | HN(R)— | —C(S)N(R)— |
| —N═C═O | H₂N— | —N(H)C(O)N(H)— |
| —N═C—O | HN(R)— | —N(H)C(O)N(R)— |
| —N═C═O | HO— | —N(H)C(O)O— |
| —N═C═O | HS— | —N(H)C(O)S— |
| —N═C═S | H₂N— | —N(H)C(S)N(H)— |
| —N═C═S | HN(R)— | —N(H)C(S)N(R)— |
| —N═C═S | HO— | —N(H)C(S)O— |
| —N═C═S | HS— | —N(H)C(S)S— |
| —SH | HS— | —S—S— |
| 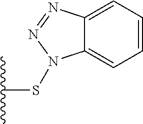 [SMILES: *Sn1nnc2ccccc21] | HS— | —S—S— |
| 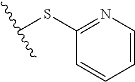 [SMILES: *Sc1ccccn1] | HS— | —S—S— |
| —CH₂—X | HS— | —CH₂—S— |
| —CH₂—X | HO— | —CH₂—O— |
| —CH₂—X | H₂N— | —CH₂—N(H)— |
| —CH═CH₂ | H₂C═CH— | —CH═CH— |

It is understood that the entries in the first and second member columns may be switched for one another so long as each member is in the same row.

### III. iRNA Agents of the Disclosure

Described herein are iRNA agents that inhibit the expression of a target gene in extrahepatic tissue, e.g., muscle tissue, e.g., skeletal muscle tissue and/or cardiac muscle tissue, or lung tissue. In one embodiment, the iRNA agent includes double stranded ribonucleic acid (dsRNA) molecules for inhibiting the expression of a target gene in a skeletal and/or cardiac muscle cell or tissue, such as a cell or tissue within a subject, e.g., a mammal, such as a human having a muscle disorder or disease, e.g., a skeletal and/or cardiac disorder or disease. Any target gene can be inhibited by the iRNA agents provided herein. In one embodiment, the target gene is any gene involved in a muscle disorder or disease, e.g., a skeletal and/or cardiac muscle disorder or disease. In one embodiment, the iRNA agent includes double stranded ribonucleic acid (dsRNA) molecules for inhibiting the expression of a target gene in a lung tissue, such as a cell or tissue within a subject, e.g., a mammal, such as a human having a muscle disorder or disease, e.g., a lung disorder or disease. Any target gene can be inhibited by the iRNA agents provided herein. In one embodiment, the target gene is any gene involved in a muscle disorder or disease, e.g., a lung disorder or disease.

Non-limiting examples of skeletal and/or cardiac muscle target genes include any genes involved in a skeletal muscle and/or a cardiac muscle disorder or disease, such as, for example, adrenoceptor beta 1 (ADRB1); calcium voltage-gated channel subunit alpha1 C (CACNA1C); calcium voltage-gated channel subunit alpha1 G (CACNA1G) (T type calcium cchannel); angiotensin II receptor type 1 (AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); Phosphodiesterase 1 (PDE1), myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); Double Homeobox 4 (DUX4), dystrophy myotonic protein kinase (DMPK), glycogen synthase 1 (GYS1), survival of motor neuron 1 (SMN1), and alpha-glucosidase (GAA).

Non-limiting examples of lung target genes include any genes involved in a lung disorder or disease, such as, for example, MUC5B, TSLP, IL33, ALOX15, AGER(RAGE), MUC5AC, and STAT6.

The iRNA agents provided herein comprise a sense strand and an antisense strand and at least one of the strands is modified for targeting delivery to extrahepatic tissue, e.g., muscle tissue, e.g., skeletal muscle tissue and/or cardiac muscle tissue, or lung tissue. In one embodiment, the iRNA agents are modified by conjugation to an alpha-v-beta-6 (αvβ6) integrin ligand.

By “integrin ligand” is meant any ligand that binds to an integrin or an integrin receptor.

Integrin ligands comprise binding sequences that are recognized and bound by integrins or integrin receptors. Various ligands, including peptides and small molecules, which bind αvβ6 integrins or αvβ6 integrin receptors (αvβ6 integrin ligands) have been designed and synthesized herein and in, e.g., U.S. Pat. No. 6,410,526, the entire contents of which are incorporated herein by reference.

An exemplary linker to conjugate a αvβ6 integrin ligand to the dsRNA agent is:

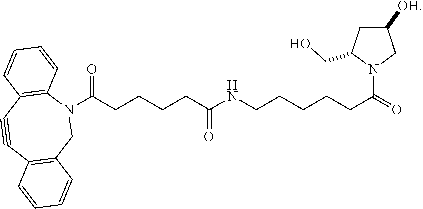 [SMILES: O=C(CCCCC(=O)N1Cc2ccccc2C#Cc2ccccc21)NCCCCCC(=O)N1C[C@H](O)C[C@H]1CO]

Exemplary αvβ6 integrin ligands include:

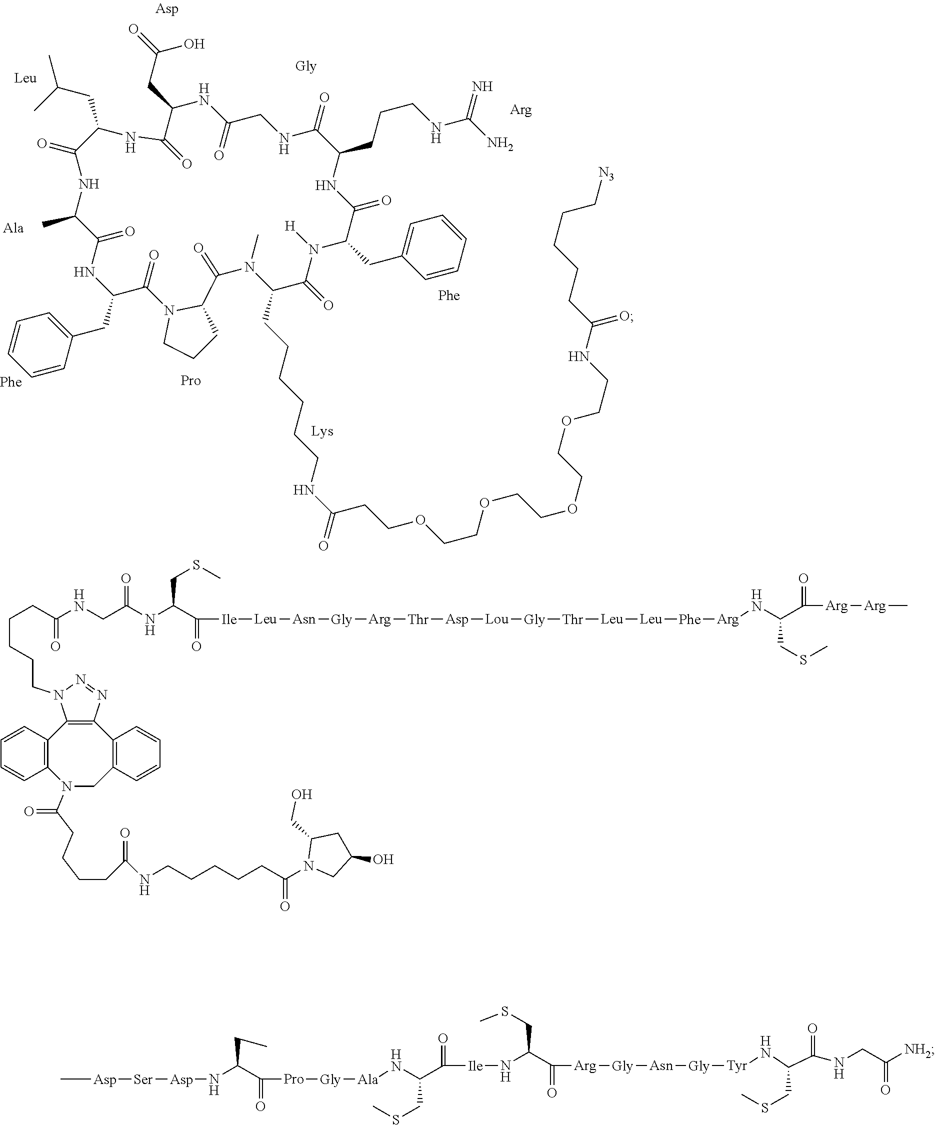

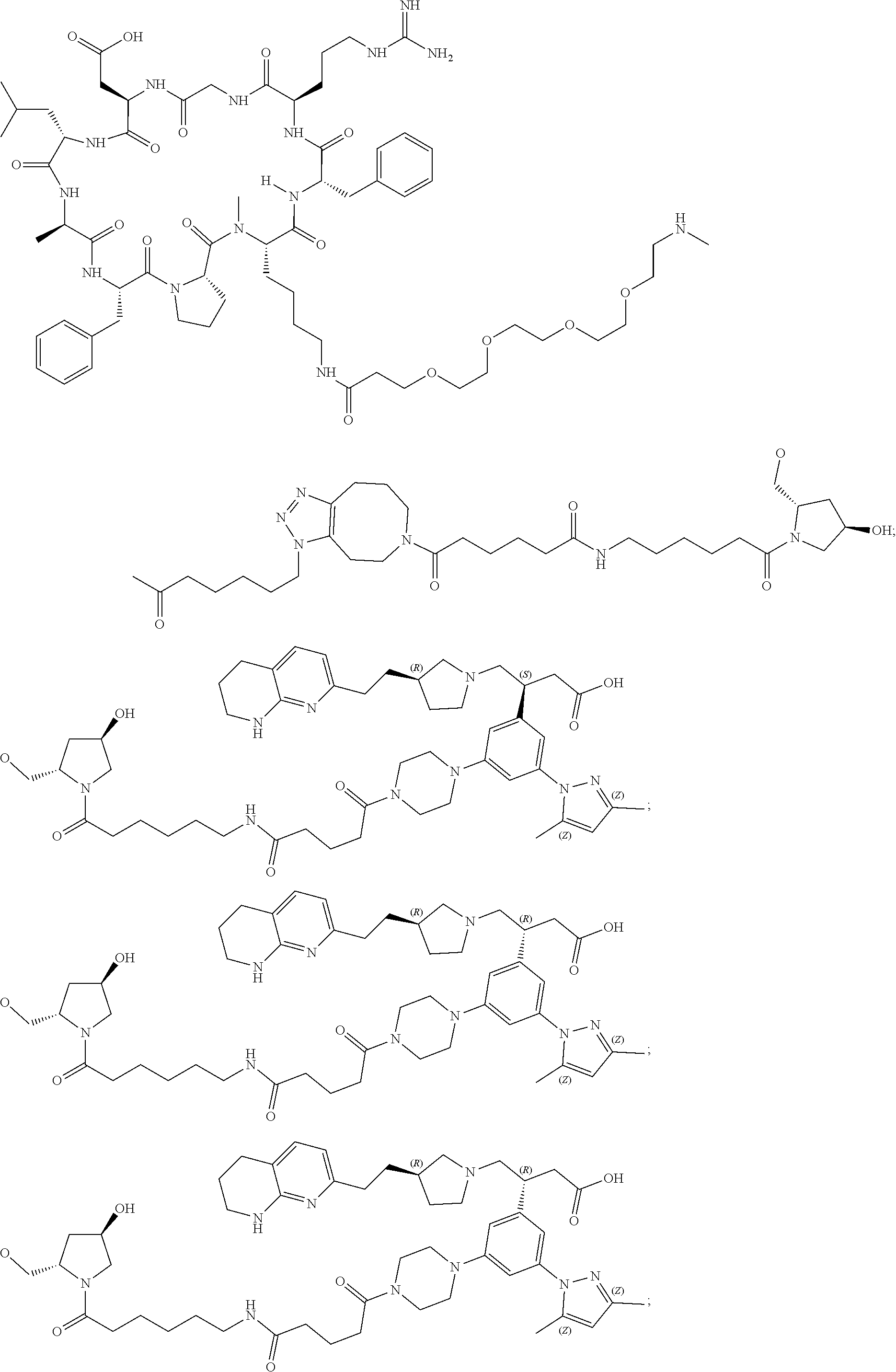

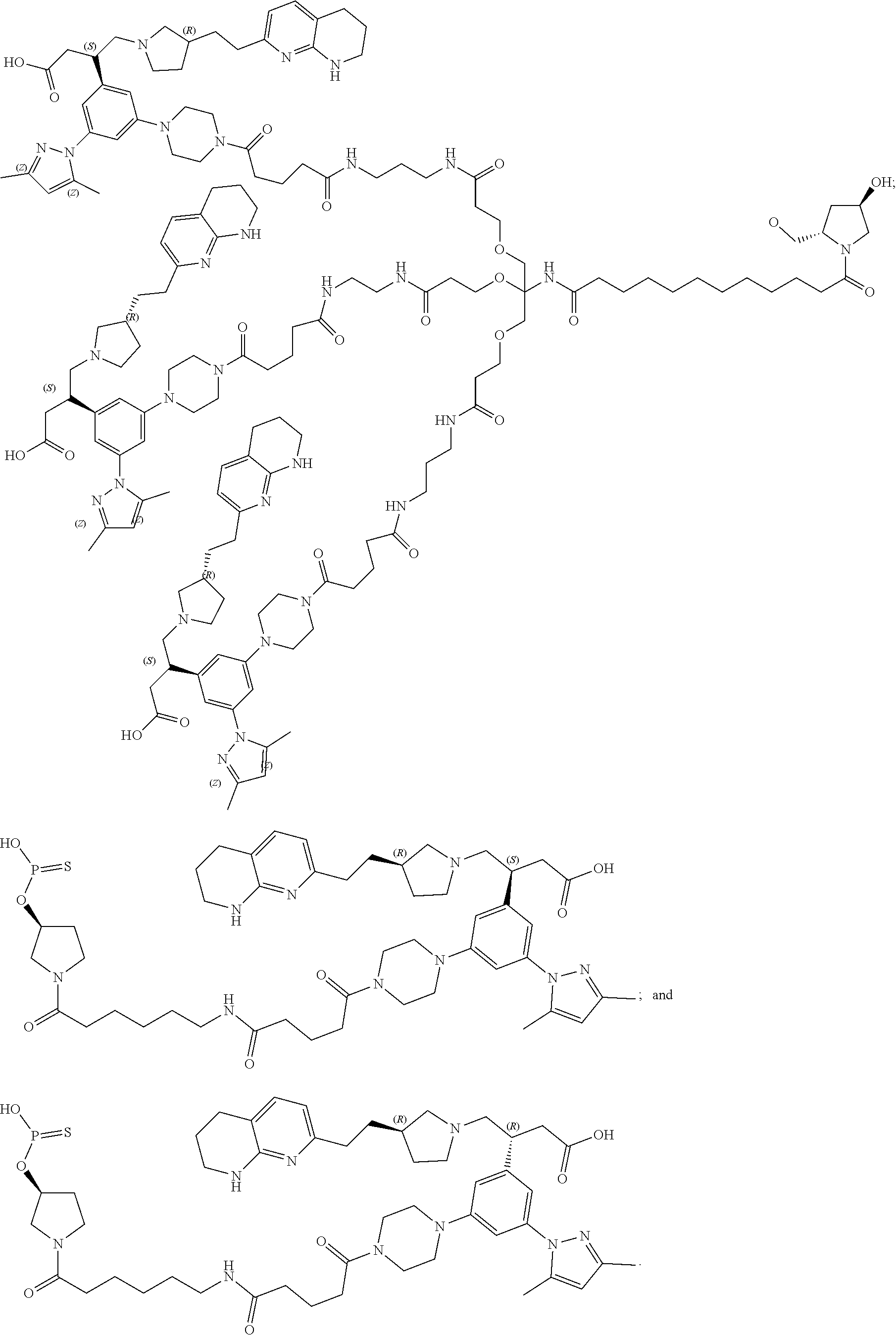

In one embodiment, at least one of the strands of the iRNA agent is conjugated to at least one αvβ6 integrin ligand. The iRNA agent may be conjugated to one, two, three, four, or more e tr integrin ligands.

In some embodiments, the αvβ6 integrin ligand is conjugated to the sense strand. The αvβ6 integrin ligand may be conjugated to the 3′-end of the sense strand, to the 5′-end of the sense strand, or to both the 5-end and the 3′-end of the sense strand.

In other embodiments, the αvβ6 integrin ligand is conjugated to the antisense strand. The αvβ6 integrin ligand may be conjugated to the 3′-end of the antisense strand, to the 5′-end of the antisense strand, or to both the 5-end and the 3′-end of the antisense strand.

The dsRNA includes an antisense strand having a region of complementarity which is complementary to at least a part of an mRNA formed in the expression of a target gene. The region of complementarity is about 15-30 nucleotides or less in length. Upon contact with a cell expressing the target gene, the RNAi agent inhibits the expression of the target gene (e.g., a human gene, a primate gene, a non-primate gene) by at least 30% as compared to a similar cell not contacted with the RNAi agent or an RNAi agent not complementary to the target gene. Expression of the gene may be assayed by, for example, a PCR or branched DNA (bDNA)-based method, or by a protein-based method, such as by immunofluorescence analysis, using, for example, western blotting or flowcytometric techniques.

A dsRNA includes two RNA strands that are complementary and hybridize to form a duplex structure under conditions in which the dsRNA will be used. One strand of a dsRNA (the antisense strand) includes a region of complementarity that is substantially complementary, or fully complementary, to a target sequence. The target sequence can be derived from the sequence of an mRNA formed during the expression of a target gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length, for example, 19-21 base pairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is 15 to 23 nucleotides in length, 19 to 23 nucleotides in length, or 25 to 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 15 to 36 base pairs, e.g., 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs, for example, 19-21 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an RNAi agent useful to target expression or a target gene is not generated in the target cell by cleavage of a larger dsRNA.

A dsRNA as described herein can further include one or more single-stranded nucleotide overhangs e.g., 1, 2, 3, or 4 nucleotides. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5-end, 3′-end or both ends of either an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the dsRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

In one aspect, a dsRNA of the disclosure includes at least two nucleotide sequences, a sense sequence and an antisense sequence. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand (passenger strand), and the second oligonucleotide is described as the corresponding antisense strand (guide strand).

In one embodiment, the substantially complementary sequences of the dsRNA are contained on separate oligonucleotides. In another embodiment, the substantially complementary sequences of the dsRNA are contained on a single oligonucleotide.

It will be understood that, although the sequences provided herein may be described as modified or conjugated sequences, the RNA of the RNAi agent of the disclosure e.g., a dsRNA of the disclosure, may comprise any one of the sequences set forth herein that is un-modified, un-conjugated, or modified or conjugated differently than described therein.

The skilled person is well aware that dsRNAs having a duplex structure of about 20 to 23 base pairs, e.g., 21, base pairs have been hailed as particularly effective in inducing RNA interference (Elbashir et al., (2001) EMBO J., 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of a target gene by not more than 10, 15, 20, 25, 30, 35, 40, 45 or 50% inhibition from a dsRNA comprising the full sequence using the in vitro assay with, e.g., skeletal and/or cardiac muscle cells or lung cells and a 10 nM concentration of the RNA agent and the PCR assay as provided in the examples herein, are contemplated to be within the scope of the present disclosure.

In addition, the RNA agents described herein identify a site(s) in a target gene mRNA transcript that is susceptible to RISC-mediated cleavage. As such, the present disclosure further features RNAi agents that target within this site(s). As used herein, an RNAi agent is said to “target within” a particular site of an mRNA transcript if the RNAi agent promotes cleavage of the mRNA transcript anywhere within that particular site. Such an RNAi agent will generally include at least about 15 contiguous nucleotides, preferably at least 19 nucleotides, from one of the sequences provided herein coupled to additional nucleotide sequences taken from the region contiguous to the selected sequence in a target gene.

### III. Modifications of the RNAi Agents of the Invention

In one embodiment, the RNA of an RNAi agent of the disclosure, e.g., a dsRNA, is chemically modified to enhance stability or other beneficial characteristics. In certain embodiments of the disclosure, substantially all of the nucleotides of an RNAi agent of the disclosure are modified. In other embodiments of the disclosure, all of the nucleotides of an RNAi agent of the disclosure are modified. RNAi agents of the disclosure in which “substantially all of the nucleotides are modified” are largely but not wholly modified and can include not more than 5, 4, 3, 2, or unmodified nucleotides. In still other embodiments of the disclosure, RNAi agents of the disclosure can include not more than 5, 4, 3, 2 or 1 modified nucleotides.

The nucleic acids featured in the disclosure can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of RNAi agents useful in the embodiments described herein include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified RNAi agent will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments of the invention, the dsRNA agents of the invention are in a free acid form. In other embodiments of the invention, the dsRNA agents of the invention are in a salt form. In one embodiment, the dsRNA agents of the invention are in a sodium salt form. In certain embodiments, when the dsRNA agents of the invention are in the sodium salt form, sodium ions are present in the agent as counterions for substantially all of the phosphodiester and/or phosphorothioate groups present in the agent. Agents in which substantially all of the phosphodiester and/or phosphorothioate linkages have a sodium counterion include not more than 5, 4, 3, 2, or 1 phosphodiester and/or phosphorothioate linkages without a sodium counterion. In some embodiments, when the dsRNA agents of the invention are in the sodium salt form, sodium ions are present in the agent as counterions for all of the phosphodiester and/or phosphorothioate groups present in the agent.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in RNAi agents, in which both the sugar and the internucleoside linkage, i.e., the backbone, of the nucleotide units are replaced with alternate groups. The nucleobase units are maintained for hybridization with an appropriate nucleic acid target compound. One such oligomeric compound, a RNA mimetic that has been shown to have excellent hybridization properties, is referred to as a peptide nucleic acid (PNA).

In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the RNAi agents of the disclosure are described in, for example, in Nielsen et al., Science, 1991, 254, 1497-1500.

Some embodiments featured in the disclosure include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506. The native phosphodiester backbone can be represented as —O—P(O)(OH)—OCH₂—.

Modified RNAs can also contain one or more substituted sugar moieties. The RNAi agents, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ alkyl, substituted alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNAi agent, or a group for improving the pharmacodynamic properties of an RNAi agent, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂₀N(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₃)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂), 2′-O-hexadecyl, and 2′-fluoro (2′-F). Similar modifications can also be made at other positions on the RNA of an RNAi agent, particularly the 3′ position of the sugar on the 3′ terminal nucleotide or in 2′-5′ linked dsRNAs and the 5′ position of 5′ terminal nucleotide. RNAi agents can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An RNAi agent of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further modified nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these modified nucleobases are particularly useful for increasing the binding affinity of the oligomeric compounds featured in the disclosure. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

An RNAi agent of the disclosure can also be modified to include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the disclosure may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides of the disclosure include without limitation nucleosides comprising a bridge between the 4′ and the 2′ ribosyl ring atoms. In certain embodiments, the antisense polynucleotide agents of the disclosure include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative US patents and US patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An RNAi agent of the disclosure can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge. In one embodiment, a constrained ethyl nucleotide is in the S conformation referred to herein as “S-cEt.”

An RNAi agent of the disclosure may also include one or more “conformationally restricted nucleotides” (“CRN”). CRN are nucleotide analogs with a linker connecting the C2′ and C4′ carbons of ribose or the C3 and -C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US 2013/0190383; and WO 2013/036868, the entire contents of each of which are hereby incorporated herein by reference.

In some embodiments, an RNAi agent of the disclosure comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and
2011/0313020, the entire contents of each of which are hereby incorporated herein by reference.

An RNAi agent of the disclosure may also include one or more “cyclohexene nucleic acids” or (“CeNA”). CeNA are nucleotide analogs with a replacement of the furanose moiety of DNA by a cyclohexene ring. Incorporation of cylcohexenyl nucleosides in a DNA chain increases the stability of a DNA/RNA hybrid. CeNA is stable against degradation in serum and a CeNA/RNA hybrid is able to activate E. Coli RNase H, resulting in cleavage of the RNA strand. (see Wang et al., Am. Chem. Soc. 2000, 122, 36, 8595-8602, hereby incorporated by reference).

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in WO 2011/005861.

Other potentially stabilizing modifications to the ends of RNA molecules include inverted nucleotides, including inverted abasic nucleotides. In one embodiment, each end of a nucleotide comprises an inverted abasic nucleotide, e.g., a 3′->3′ linked nucleotide at the 3′-end of the oligonucleotide and a 5′->5′ linked nucleotide at the 5′-end of the oligonucleotide. In each instance, independently, the inverted abasic nucleotide can be linked via a phosphodiester or phosphothioate internucleotide linkage. Other modifications of an RNAi agent of the disclosure include a 5′ phosphate or 5′ phosphate mimic, e.g., a 5′-terminal phosphate or phosphate mimic on the antisense strand of an RNAi agent. Suitable phosphate mimics are disclosed in, for example US 2012/0157511, the entire contents of which are incorporated herein by reference.

### A. Modified RNAi Agents Comprising Motifs of the Invention

In certain aspects of the disclosure, the double-stranded RNAi agents of the disclosure include agents with chemical modifications as disclosed, for example, in WO 2013/075035, the entire contents of which are incorporated herein by reference. As shown herein and in WO 2013/075035, a superior result may be obtained by introducing one or more motifs of three identical modifications on three consecutive nucleotides into a sense strand or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense or antisense strand. The RNAi agent may be optionally modified with a (S)-glycol nucleic acid (GNA) modification, for instance on one or more residues of the antisense strand. The resulting RNAi agents present superior gene silencing activity.

Accordingly, the disclosure provides double stranded RNAi agents capable of inhibiting the expression of a target gene in vivo. The RNAi agent comprises a sense strand and an antisense strand. Each strand of the RNAi agent may be 15-30 nucleotides in length. For example, each strand may be 16-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. In certain embodiments, each strand is 19-23 nucleotides in length.

The sense strand and antisense strand typically form a duplex double stranded RNA (“dsRNA”), also referred to herein as an “RNAi agent.” The duplex region of an RNAi agent may be 15-30 nucleotide pairs in length. For example, the duplex region can be 16-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length. In another embodiment, the duplex region is 19-21 nucleotide pairs in length.

In one embodiment, the RNAi agent may contain one or more overhang regions or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In another embodiment, the nucleotide overhang region is 2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-O-methyl, thymidine (T), and any combinations thereof.

For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The dsRNAi agent may contain only a single overhang, which can strengthen the interference activity of the RNAi, without affecting its overall stability. For example, the single-stranded overhang may be located at the 3-terminal end of the sense strand or, alternatively, at the 3-terminal end of the antisense strand. The RNAi may also have a blunt end, located at the 5′-end of the antisense strand (i.e., the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double blunt-ended of 19 nucleotides in length, wherein the sense strand contains at least one motif of three 2′-F modifications on three consecutive nucleotides at positions 7, 8, and 9 from the 5′ end. The antisense strand contains at least one motif of three 2′-O-methyl modifications on three consecutive nucleotides at positions 11, 12, and 13 from the 5′ end.

In another embodiment, the RNAi agent is a double blunt-ended of 20 nucleotides in length, wherein the sense strand contains at least one motif of three 2′-F modifications on three consecutive nucleotides at positions 8, 9, and 10 from the 5′ end. The antisense strand contains at least one motif of three 2′-O-methyl modifications on three consecutive nucleotides at positions 11, 12, and 13 from the 5′ end.

In yet another embodiment, the RNAi agent is a double blunt-ended of 21 nucleotides in length, wherein the sense strand contains at least one motif of three 2′-F modifications on three consecutive nucleotides at positions 9, 10, and 11 from the 5′ end. The antisense strand contains at least one motif of three 2′-O-methyl modifications on three consecutive nucleotides at positions 11, 12, and 13 from the 5′ end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, and 11 from the 5′ end; the antisense strand contains at least one motif of three 2′-O-methyl modifications on three consecutive nucleotides at positions 11, 12, and 13 from the 5′ end, wherein one end of the RNAi agent is blunt, while the other end comprises a 2 nucleotide overhang. Preferably, the 2 nucleotide overhang is at the 3′-end of the antisense strand.

When the 2 nucleotide overhang is at the 3′-end of the antisense strand, there may be two phosphorothioate internucleotide linkages between the terminal three 3′-nucleotides of the antisense strand, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 2′-fluoro, e.g., in an alternating motif.

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the dsRNAi agent comprises sense and antisense strands, wherein the dsRNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, and 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, and 11 positions; 10, 11, and 12 positions; 11, 12, and 13 positions; 12, 13, and 14 positions; or 13, 14, and 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other, then the chemistry of the motifs are distinct from each other and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two, or three nucleotides in the duplex region.

In one embodiment, the RNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxythimidine (dT). In another embodiment, the nucleotide at the 3′-end of the antisense strand is deoxythimidine (dT). In one embodiment, there is a short sequence of deoxythimidine nucleotides, for example, two dT nucleotides on the 3′-end of the sense or antisense strand.

In one embodiment, the sense strand sequence may be represented by Formula (I):

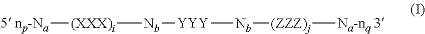

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein Nb and Y do not have the same modification; and
XXX, YYY and ZZZ each independently represent one motif of three identical modifications on three consecutive nucleotides. Preferably YYY is all 2′-F modified nucleotides.

In one embodiment, the Nₐ or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11, 12 or 11, 12, 13) of—the sense strand, the count starting from the 1^{st} nucleotide, from the 5′-end; or optionally, the count starting at the 1^{st} paired nucleotide within the duplex region, from the 5′-end.

In one embodiment, i is 1 and j is 0, or i is 0 and j is 1, or both i and j are 1. The sense strand can therefore be represented by the following formulas:

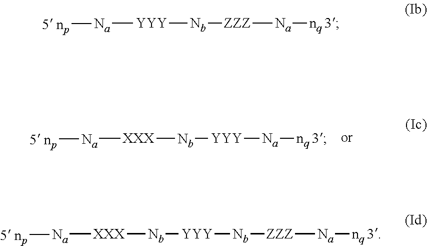

When the sense strand is represented by Formula (Ib), N_{b} represents an oligonucleotide sequence comprising 0-10, 0-7, 0-5, 0-4, 0-2 or 0 modified nucleotides.

Each Nₐ independently can represent an oligonucleotide sequence comprising 2-20, 2-15, or 2-10 modified nucleotides.

When the sense strand is represented as Formula (Ic), N_{b} represents an oligonucleotide sequence comprising 0-10, 0-7, 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as Formula (Id), each N_{b} independently represents an oligonucleotide sequence comprising 0-10, 0-7, 0-5, 0-4, 0-2 or 0 modified nucleotides. Preferably, N_{b} is 0, 1, 2, 3, 4, 5 or 6. Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

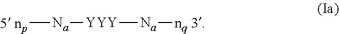

When the sense strand is represented by Formula (Ia), each Nₐ independently can represent an oligonucleotide sequence comprising 2-20, 2-15, or 2-10 modified nucleotides.

In one embodiment, the antisense strand sequence of the RNAi may be represented by Formula (II):

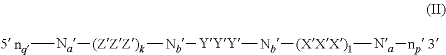

wherein:
k and l are each independently 0 or 1;
p′ and q′ are each independently 0-6;
   each Nₐ′ independently represents an oligonucleotide sequence comprising 0-25 modified nucleotides,
   each sequence comprising at least two differently modified nucleotides;
   each N_{b}′ independently represents an oligonucleotide sequence comprising 0-10 modified nucleotides;
   each nₚ′ and n_{q}′ independently represent an overhang nucleotide;
   wherein N_{b}′ and Y′ do not have the same modification;
   and X′X′X′, Y′Y′Y′ and Z′Z′Z′ each independently represent one motif of three identical modifications on three consecutive nucleotides.
   In one embodiment, the Nₐ′ or N_{b}′ comprise modifications of alternating pattern.

The Y′Y′Y′ motif occurs at or near the cleavage site of the antisense strand. For example, when the RNAi agent has a duplex region of 17-23 nucleotide in length, the Y′Y′Y′ motif can occur at positions 9, 10, 11; 10, 11, 12; 11, 12, 13; 12, 13, 14; or 13, 14, 15 of the antisense strand, with the count starting from the 1^{st} nucleotide, from the 5′-end; or optionally, the count starting at the 1^{st} paired nucleotide within the duplex region, from the 5′-end. Preferably, the Y′Y′Y′ motif occurs at positions 11, 12, 13.

In one embodiment, Y′Y′Y′ motif is all 2′-OMe modified nucleotides.

In one embodiment, k is 1 and 1 is 0, or k is 0 and 1 is 1, or both k and l are 1.

The antisense strand can therefore be represented by the following formulas:

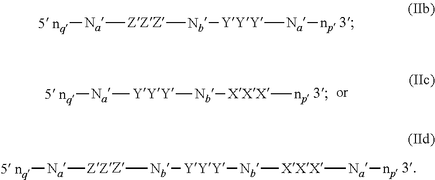

When the antisense strand is represented by Formula (IIb), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as Formula (IIc), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as Formula (IId), each N_{b}′ independently represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5 or 6.

In other embodiments, k is 0 and 1 is 0 and the antisense strand may be represented by the formula:

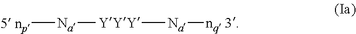

When the antisense strand is represented as Formula (IIa), each Nₐ′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, HNA, CeNA, 2′-methoxyethyl, 2′-O-methyl, 2′-O-allyl, 2′-C-allyl, 2′-hydroxyl, or 2′-fluoro.

For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1^{st} nucleotide from the 5′-end, or optionally, the count starting at the 1^{st} paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the 1^{st} nucleotide from the 5′-end, or optionally, the count starting at the 1^{st} paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with an antisense strand being represented by any one of formulas (IIa), (IIb), (IIc), and (IId), respectively.

Accordingly, the RNAi agents for use in the methods of the disclosure may comprise a sense strand and an antisense strand, each strand having 14 to 30 nucleotides, the RNAi duplex represented by Formula (III):

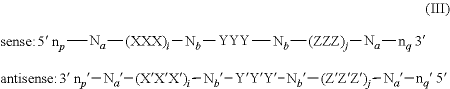

wherein:
i, j, k, and l are each independently 0 or 1;
p, p′, q, and q′ are each independently 0-6;
each Nₐ and Nₐ′ independently represents an oligonucleotide sequence comprising 0-25 modified nucleotides, each sequence comprising at least two differently modified nucleotides;
each N_{b} and N_{b}′ independently represents an oligonucleotide sequence comprising 0-10 modified nucleotides;
wherein
each nₚ′, nₚ, n_{q}′, and n_{q}, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and 1 is 0; or k is 1 and 1 is 0; k is 0 and 1 is 1; or both k and l are 0; or both k and l are 1.

Exemplary combinations of the sense strand and antisense strand forming an RNAi duplex include the formulas below:

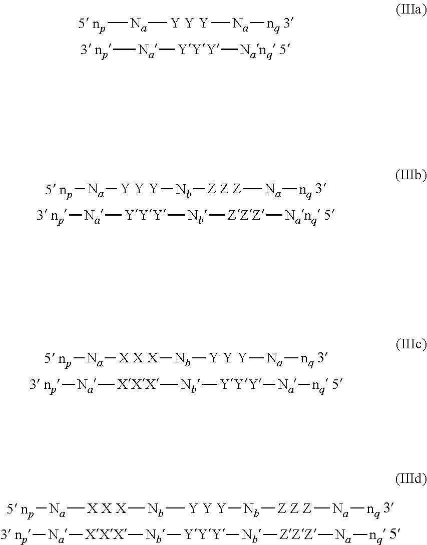

When the RNAi agent is represented by Formula (IIIa), each Nₐ independently represents an oligonucleotide sequence comprising 2-20, 2-15, or 2-10 modified nucleotides.

When the RNAi agent is represented by Formula (IIIb), each N_{b} independently represents an oligonucleotide sequence comprising 1-10, 1-7, 1-5 or 1-4 modified nucleotides. Each Nₐ independently represents an oligonucleotide sequence comprising 2-20, 2-15, or 2-10 modified nucleotides.

When the RNAi agent is represented as Formula (IIIc), each N_{b}, N_{b}′ independently represents an oligonucleotide sequence comprising 0-10, 0-7, 0-10, 0-7, 0-5, 0-4, 0-2 or 0 modified nucleotides. Each Nₐ independently represents an oligonucleotide sequence comprising 2-20, 2-15, or 2-10 modified nucleotides.

When the RNAi agent is represented as Formula (IIId), each N_{b}, N_{b}′ independently represents an oligonucleotide sequence comprising 0-10, 0-7, 0-10, 0-7, 0-5, 0-4, 0-2 or 0 modified nucleotides. Each Nₐ, Nₐ′ independently represents an oligonucleotide sequence comprising 2-20, 2-15, or 2-10 modified nucleotides. Each of Nₐ, Nₐ′, N_{b} and N_{b}′ independently comprises modifications of alternating pattern.

In one embodiment, when the RNAi agent is represented by Formula (IIId), the Nₐ modifications are 2′-O-methyl or 2′-fluoro modifications. In another embodiment, when the RNAi agent is represented by Formula (IIId), the Nₐ modifications are 2′-O-methyl or 2′-fluoro modifications and nₚ′>0 and at least one nₚ′ is linked to a neighboring nucleotide a via phosphorothioate linkage. In yet another embodiment, when the RNAi agent is represented by Formula (IIId), the Nₐ modifications are 2′-O-methyl or 2′-fluoro modifications, nₚ′>0 and at least one nₚ′ is linked to a neighboring nucleotide via phosphorothioate linkage, and the sense strand is conjugated to one or more ligands attached through a bivalent or trivalent branched linker (described below). In another embodiment, when the RNAi agent is represented by Formula (IIId), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more ligands optionally attached through a bivalent or trivalent branched linker.

In one embodiment, when the RNAi agent is represented by Formula (IIIa), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more ligands attached through a bivalent or trivalent branched linker.

In one embodiment, the RNAi agent is a multimer containing at least two duplexes represented by Formula (III), (IIIa), (IIIb), (IIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by Formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by Formula (III), (IIIa), (IIIb), (IIIc), and (IIId) are linked to each other at the 5′ end, and one or both of the 3′ ends and are optionally conjugated to a ligand. Each of the agents can target the same gene or two different genes; or each of the agents can target same gene at two different target sites.

Various publications describe multimeric RNAi agents that can be used in the methods of the disclosure. Such publications include WO2007/091269, WO2010/141511, WO2007/117686, WO2009/014887, and WO2011/031520; and U.S. Pat. No. 7,858,769, the entire contents of each of which are hereby incorporated herein by reference.

In certain embodiments, the compositions and methods of the disclosure include a vinyl phosphonate (VP) modification of an RNAi agent as described herein. I

In exemplary embodiments, a 5′-vinyl phosphonate modified nucleotide of the disclosure has the structure:

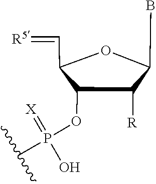

wherein X is O or S (e.g. S);
R^{P} is hydrogen, hydroxy, fluoro, methoxy, or 2-methoxyethoxy (MOE);
R^{5′} is ═C(H)—P(O)(OH)₂ and the double bond between the C5′ carbon and R5′ is in the E or Z orientation (e.g., E orientation); and
B is a nucleobase or a modified nucleobase, optionally where B is adenine, guanine, cytosine, thymine, or uracil (e.g., uracil).

In one embodiment, R^{5′} is ═C(H)—P(O)(OH)₂ and the double bond between the C5′ carbon and R5′ is in the E orientation.

In another embodiment, R is methoxy and R^{5′} is ═C(H)—P(O)(OH)₂ and the double bond between the C5′ carbon and R^{5′} is in the E orientation.

In another embodiment, X is S, R is methoxy, and R^{5′} is ═C(H)—P(O)(OH)₂ and the double bond between the C5′ carbon and R^{5′} is in the E orientation.

In another embodiment, X is S, R is methoxy, and R^{5′} is ═C(H)—P(O)(OH)₂, the double bond between the C5′ carbon and R^{5′} is in the E orientation, and B is uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, at the 5′ end of the antisense strand of the dsRNA.

Vinyl phosphate modifications are also contemplated for the compositions and methods of the instant disclosure. An exemplary vinyl phosphate structure is:

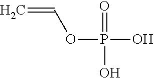

For example, when the phosphate mimic is a 5′-vinyl phosphate, the 5′-terminal nucleotide can have the immediately preceding structure, where the phosphonate group is replaced by a phosphate.
i. Thermally Destabilizing Modifications

In certain embodiments, a dsRNA molecule can be optimized for RNA interference by incorporating thermally destabilizing modifications in the seed region of the antisense strand. As used herein “seed region” means at positions 2-9 of the 5′-end of the referenced strand. For example, thermally destabilizing modifications can be incorporated in the seed region of the antisense strand to reduce or inhibit off-target gene silencing.

The term “thermally destabilizing modification(s)” includes modification(s) that would result with a dsRNA with a lower overall melting temperature (Tₘ) than the Tₘ of the dsRNA without having such modification(s). For example, the thermally destabilizing modification(s) can decrease the Tₘ of the dsRNA by 1-4° C., such as one, two, three or four degrees Celsius. And, the term “thermally destabilizing nucleotide” refers to a nucleotide containing one or more thermally destabilizing modifications.

It has been discovered that dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, one or more thermally destabilizing modification(s) of the duplex is/are located in positions 2-9, or preferably positions 4-8, from the 5′-end of the antisense strand. In some further embodiments, the thermally destabilizing modification(s) of the duplex is/are located at position 6, 7 or 8 from the 5′-end of the antisense strand. In still some further embodiments, the thermally destabilizing modification of the duplex is located at position 7 from the 5′-end of the antisense strand. In some embodiments, the thermally destabilizing modification of the duplex is located at position 2, 3, 4, 5 or 9 from the 5′-end of the antisense strand.

The thermally destabilizing modifications can include, but are not limited to, abasic modification; mismatch with the opposing nucleotide in the opposing strand; and sugar modification such as 2′-deoxy modification, a 2′-5′ linked nucleotide (“3′-RNA’), or acyclic nucleotide, e.g., unlocked nucleic acids (UNA) or glycol nucleic acid (GNA).

Exemplified abasic modifications include, but are not limited to the following:

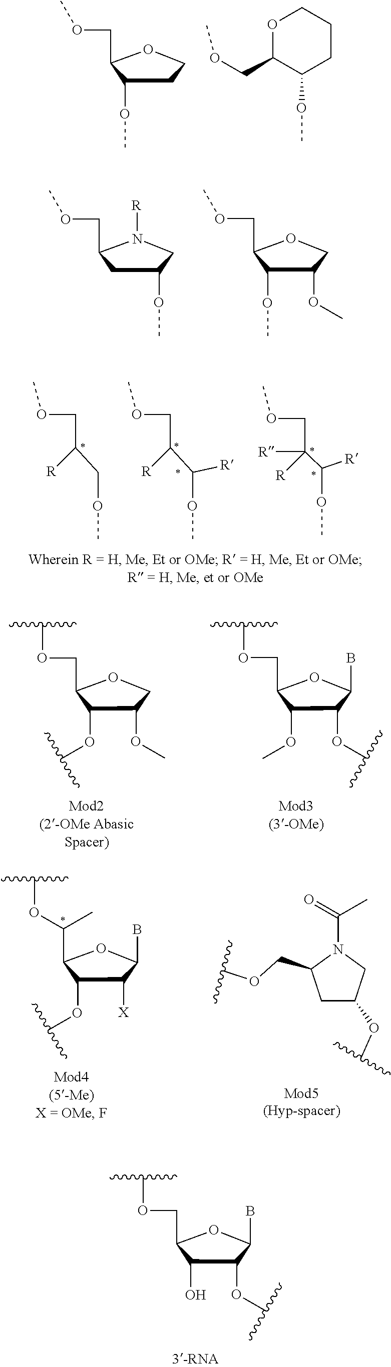

wherein B is a modified or unmodified nucleobase.

Exemplified sugar modifications include, but are not limited to the following:

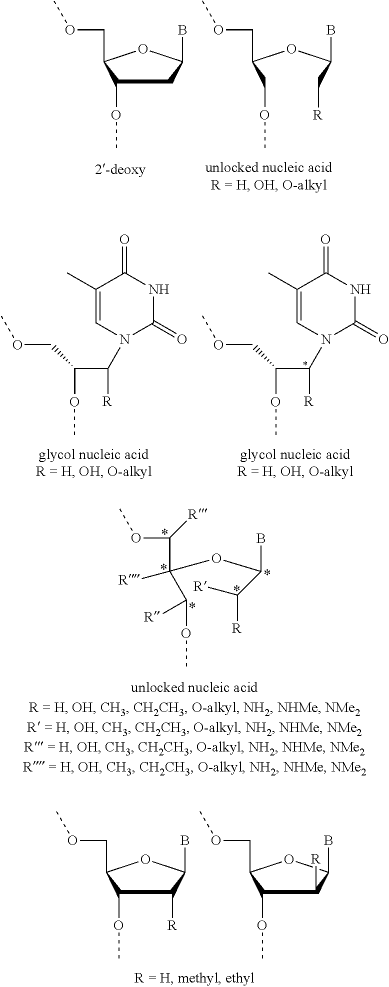

wherein B is a modified or unmodified nucleobase.

In some embodiments the thermally destabilizing modification of the duplex is selected from the group consisting of:

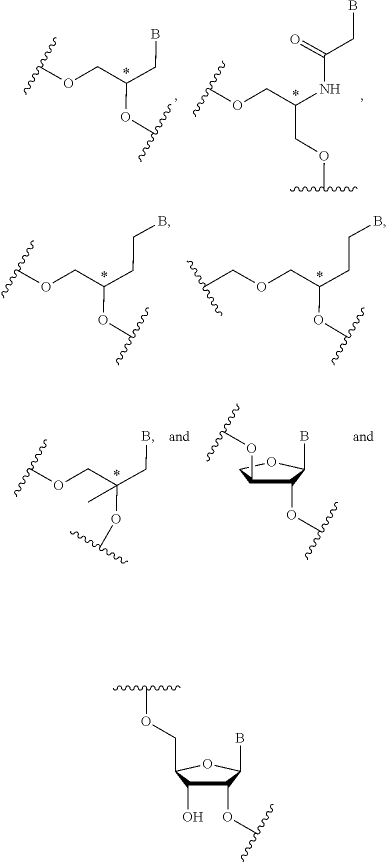

wherein B is a modified or unmodified nucleobase and the asterisk on each structure represents either R, S or racemic.

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent or at least one of ribose carbons or oxygen (e.g., C1′, C2′, C3′, C4′ or O4′) are independently or in combination absent from the nucleotide. In some embodiments, acyclic nucleotide

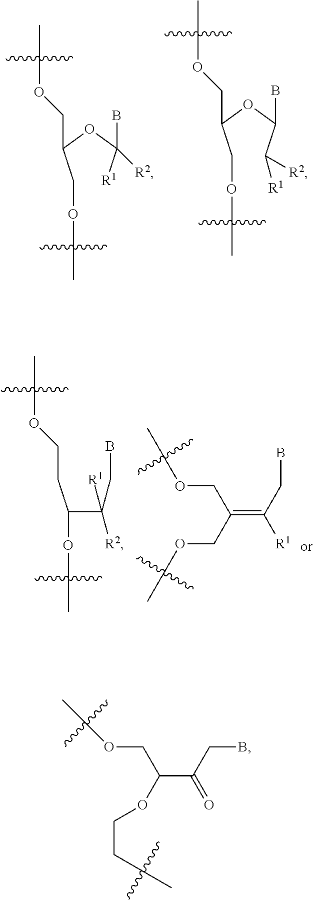

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

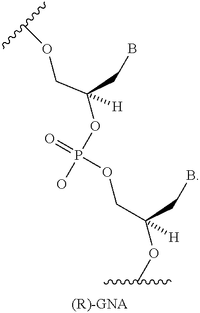

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W—C H-bonding to complementary base on the target mRNA such as:

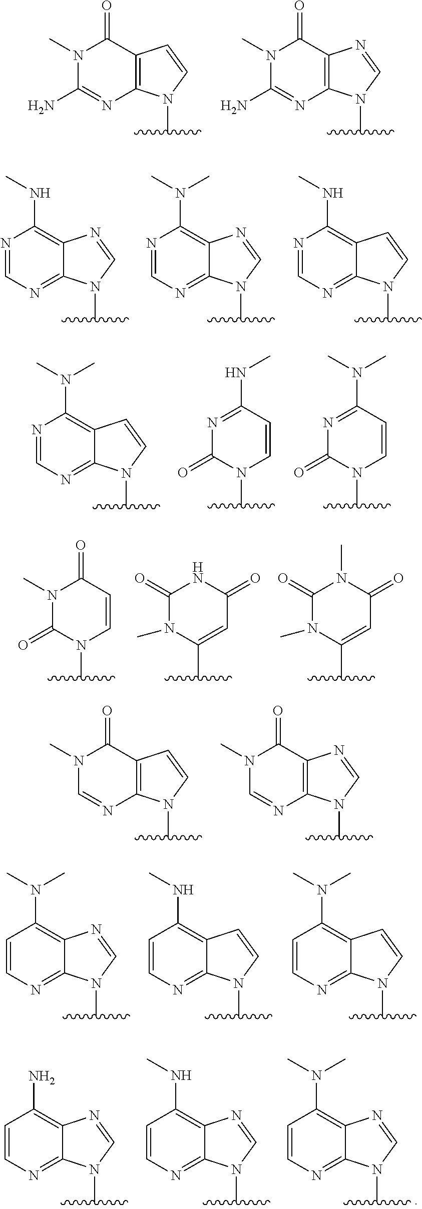

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

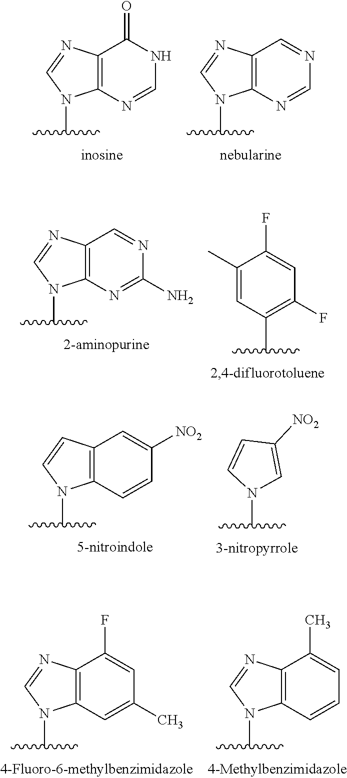

In some embodiments, the thermally destabilizing modification of the duplex in the seed region of the antisense strand includes one or more α-nucleotide complementary to the base on the target mRNA, such as:

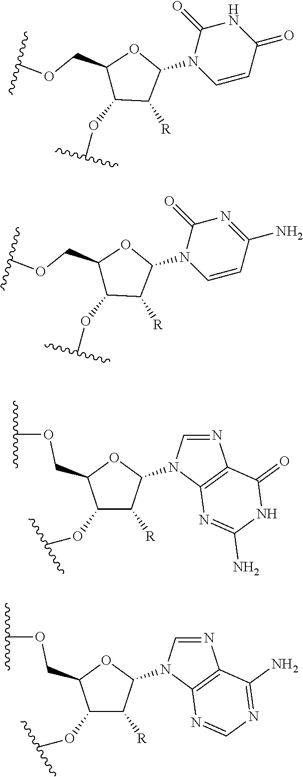

wherein R is H, OH, OCH₃, F, NH₂, NHMe, NMe₂ or O-alkyl.

Exemplary phosphate modifications known to decrease the thermal stability of dsRNA duplexes compared to natural phosphodiester linkages are:

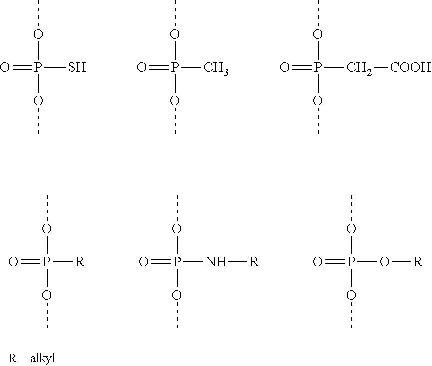

The alkyl for the R group can be a C₁-C₆alkyl. Specific alkyls for the R group include, but are not limited to methyl, ethyl, propyl, isopropyl, butyl, pentyl and hexyl.

As the skilled artisan will recognize, in view of the functional role of nucleobases is defining specificity of an RNAi agent of the disclosure, while nucleobase modifications can be performed in the various manners as described herein, e.g., to introduce destabilizing modifications into an RNAi agent of the disclosure, e.g., for purpose of enhancing on-target effect relative to off-target effect, the range of modifications available and, in general, present upon RNAi agents of the disclosure tends to be much greater for non-nucleobase modifications, e.g., modifications to sugar groups or phosphate backbones of polyribonucleotides. Such modifications are described in greater detail in other sections of the instant disclosure and are expressly contemplated for RNAi agents of the disclosure, either possessing native nucleobases or modified nucleobases as described above or elsewhere herein.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14, and 16 from the 5′-end. In some other embodiments, the antisense comprises stabilizing modifications at positions 2, 6, 14, and 16 from the 5′-end. In still some other embodiments, the antisense comprises stabilizing modifications at positions 2, 14, and 16 from the 5′-end. In each of the preceding, the stabilizing modification can be a 2′-fluoro modification.

In some other embodiments, the antisense comprises 2′-H modifications (DNA) at positions 2, 5, 7, 12, 14, and 16 from the 5′-end of the antisense strand. In still some other embodiments, the antisense comprises 2′-H modifications (DNA) at positions 2, 5, 7, and 12 from the 5′-end, and a 2′-fluoro modification at position 14 from the 5′-end of the antisense strand. In other embodiments, the antisense strand is paired to a sense strand having 2′-fluoro modifications at positions 9, 10, and 11 from the 5′-end of the sense strand (or opposite to positions 11, 12, and 13 of the antisense strand). In other embodiments, the antisense strand is paired to a sense strand having 2′-fluoro modifications at positions 7, 9, 10, and 11 from the 5′-end of the sense strand (or opposite to positions 11, 12, 13, and 15 of the antisense strand).

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position -1 or +1 from the position of the destabilizing modification. In some embodiments, the antisense strand comprises a stabilizing modification at each of the 5′-end and the 3′-end of the destabilizing modification, i.e., positions -1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) stabilizing modifications (e.g., 2′-fluoro modifications). Without limitations, a stabilizing modification in the sense strand can be present at any positions. In some embodiments, the sense strand comprises stabilizing modifications at positions 7, 10, and 11 from the 5′-end. In some other embodiments, the sense strand comprises stabilizing modifications at positions 7, 9, 10, and 11 from the 5′-end. In some embodiments, the sense strand comprises stabilizing modifications at positions opposite or complementary to positions 11, 12, and 15 of the antisense strand, counting from the 5′-end of the antisense strand. In some other embodiments, the sense strand comprises stabilizing modifications (e.g., 2′-fluoro modifications) at positions opposite or complementary to positions 11, 12, 13, and 15 of the antisense strand, counting from the 5′-end of the antisense strand. In some embodiments, the sense strand comprises a block of two, three, or four stabilizing modifications.

In some other embodiments, the sense strand comprises stabilizing modifications (e.g., 2′-fluoro modifications) at positions opposite or complementary to positions 11, 12, and 13 of the antisense strand, counting from the 5′-end of the antisense strand.

In some embodiments, the sense strand does not comprise a stabilizing modification in position opposite or complementary to the thermally destabilizing modification of the duplex in the antisense strand.

Exemplary thermally stabilizing modifications include, but are not limited to, 2′-fluoro modifications. Other thermally stabilizing modifications include, but are not limited to, LNA.

In some embodiments, the dsRNA of the disclosure comprises at least four (e.g., four, five, six, seven, eight, nine, ten, or more) 2′-fluoro nucleotides. Without limitations, the 2′-fluoro nucleotides all can be present in one strand. In some embodiments, both the sense and the antisense strands comprise at least two 2′-fluoro nucleotides. The 2′-fluoro modification can occur on any nucleotide of the sense strand or antisense strand. For instance, the 2′-fluoro modification can occur on every nucleotide on the sense strand or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

In some embodiments, the antisense strand comprises at least two (e.g., two, three, four, five, six, seven, eight, nine, ten, or more) 2′-fluoro nucleotides. Without limitations, a 2′-fluoro modification in the antisense strand can be present at any positions. In some embodiments, the antisense comprises 2′-fluoro nucleotides at positions 2, 6, 8, 9, 14, and 16 from the 5′-end. In some other embodiments, the antisense comprises 2′-fluoro nucleotides at positions 2, 6, 14, and 16 from the 5′-end. In still some other embodiments, the antisense comprises 2′-fluoro nucleotides at positions 2, 14, and 16 from the 5′-end.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i.e., at position -1 or +1 from the position of the destabilizing modification. In some embodiments, the antisense strand comprises a 2′-fluoro nucleotide at each of the 5′-end and the 3′-end of the destabilizing modification, i.e., positions -1 and +1 from the position of the destabilizing modification.

In some embodiments, the antisense strand comprises at least two 2′-fluoro nucleotides at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) 2′-fluoro nucleotides. Without limitations, a 2′-fluoro modification in the sense strand can be present at any positions. In some embodiments, the antisense comprises 2′-fluoro nucleotides at positions 7, 10, and 11 from the 5′-end. In some other embodiments, the sense strand comprises 2′-fluoro nucleotides at positions 7, 9, 10, and 11 from the 5′-end. In some embodiments, the sense strand comprises 2′-fluoro nucleotides at positions opposite or complementary to positions 11, 12, and 15 of the antisense strand, counting from the 5′-end of the antisense strand. In some other embodiments, the sense strand comprises 2′-fluoro nucleotides at positions opposite or complementary to positions 11, 12, 13, and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complementary to the thermally destabilizing modification of the duplex in the antisense strand.

In some embodiments, the dsRNA molecule of the disclosure comprises a 21 nucleotides (nt) sense strand and a 23 nucleotides (nt) antisense, wherein the antisense strand contains at least one thermally destabilizing nucleotide, where the at least one (e.g., only one) thermally destabilizing nucleotide occurs in the seed region of the antisense strand (i.e., at position 2-9 of the 5′-end of the antisense strand), wherein one end of the dsRNA is blunt, while the other end is comprises a 2 nt overhang, and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; and (vii) the dsRNA comprises a blunt end at 5′-end of the antisense strand. Preferably, the 2 nt overhang is at the 3′-end of the antisense.

In some embodiments, the dsRNA molecule of the disclosure comprising a sense and antisense strands, wherein: the sense strand is 25-30 nucleotide residues in length, wherein starting from the 5′ terminal nucleotide (position 1), positions 1 to 23 of said sense strand comprise at least 8 ribonucleotides; antisense strand is 36-66 nucleotide residues in length and, starting from the 3′ terminal nucleotide, at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the antisense strand contains at least one thermally destabilizing nucleotide, where at least one thermally destabilizing nucleotide is in the seed region of the antisense strand (i.e. at position 2-9 of the 5′-end of the antisense strand). For example, the thermally destabilizing nucleotide occurs between positions opposite or complementary to positions 14-17 of the 5′-end of the sense strand, and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2, 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA comprises a duplex region of 12-30 nucleotide pairs in length.

In some embodiments, the dsRNA molecule of the disclosure comprises a sense and antisense strands, wherein said dsRNA molecule comprises a sense strand having a length which is at least 25 and at most 29 nucleotides and an antisense strand having a length which is at most 30 nucleotides with the sense strand comprises a modified nucleotide that is susceptible to enzymatic degradation at position 11 from the 5′ end, wherein the 3′ end of said sense strand and the 5′ end of said antisense strand form a blunt end and said antisense strand is 1-4 nucleotides longer at its 3′ end than the sense strand, wherein the duplex region which is at least 25 nucleotides in length, and said antisense strand is sufficiently complementary to a target mRNA along at least 19 nt of said antisense strand length to reduce target gene expression when said dsRNA molecule is introduced into a mammalian cell, and wherein dicer cleavage of said dsRNA preferentially results in an siRNA comprising said 3′ end of said antisense strand, thereby reducing expression of the target gene in the mammal, wherein the antisense strand contains at least one thermally destabilizing nucleotide, where the at least one thermally destabilizing nucleotide is in the seed region of the antisense strand (i.e. at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2, 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA has a duplex region of 12-29 nucleotide pairs in length.

In some embodiments, every nucleotide in the sense strand and antisense strand of the dsRNA molecule may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, F-HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy, 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with 2′-O-methyl nucleotide, 2′-deoxy nucleotide, 2′-deoxy-2′-fluoro nucleotide, 2′-O—N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand.

In some embodiments, the dsRNA molecule of the disclosure comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA molecule of the disclosure comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

The dsRNA molecule of the disclosure may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA molecule comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of three phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of four phosphorothioate or methylphosphonate internucleotide linkages separated by 1, 2, 3, 4, 5, 6, 7, 8, 9, 10, 11, 12, 13, or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of five phosphorothioate or methylphosphonate internucleotide linkages separated by 1, 2, 3, 4, 5, 6, 7, 8, 9, 10, 11, or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of six phosphorothioate or methylphosphonate internucleotide linkages separated by 1, 2, 3, 4, 5, 6, 7, 8, 9, or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleotide linkages separated by 1, 2, 3, 4, 5, 6, 7, or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleotide linkages separated by 1, 2, 3, 4, 5, or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleotide linkages separated by 1, 2, 3, or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA molecule of the disclosure further comprises one or more phosphorothioate or methylphosphonate internucleotide linkage modification within 1-10 of the termini position(s) of the sense or antisense strand. For example, at least 2, 3, 4, 5, 6, 7, 8, 9, or 10 nucleotides may be linked through phosphorothioate or methylphosphonate internucleotide linkage at one end or both ends of the sense or antisense strand.

In some embodiments, the dsRNA molecule of the disclosure further comprises one or more phosphorothioate or methylphosphonate internucleotide linkage modification within 1-10 of the internal region of the duplex of each of the sense or antisense strand. For example, at least 2, 3, 4, 5, 6, 7, 8, 9, or 10 nucleotides may be linked through phosphorothioate methylphosphonate internucleotide linkage at position 8-16 of the duplex region counting from the 5′-end of the sense strand; the dsRNA molecule can optionally further comprise one or more phosphorothioate or methylphosphonate internucleotide linkage modification within 1-10 of the termini position(s).

In some embodiments, the dsRNA molecule of the disclosure further comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA molecule of the disclosure further comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 (counting from the 5′-end) of the sense strand, and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 (counting from the 5′-end) of the sense strand, and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some embodiments, compound of the disclosure comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, compound of the disclosure comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, compound of the disclosure comprises a 5′-block is an Sp block wherein each sugar moiety comprises a 2′-F modification. In some embodiments, a 5′-block is an Sp block wherein each of internucleotidic linkage is a modified internucleotidic linkage and each sugar moiety comprises a 2′-F modification. In some embodiments, a 5′-block is an Sp block wherein each of internucleotidic linkage is a phosphorothioate linkage and each sugar moiety comprises a 2′-F modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-F modification. In some embodiments, a 3′-block is an Sp block wherein each of internucleotidic linkage is a modified internucleotidic linkage and each sugar moiety comprises a 2′-F modification. In some embodiments, a 3′-block is an Sp block wherein each of internucleotidic linkage is a phosphorothioate linkage and each sugar moiety comprises a 2′-F modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, compound of the disclosure comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

In some embodiments, the antisense strand comprises phosphorothioate internucleotide linkages between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 1, 2, 3, 4 or 5 phosphorothioate internucleotide linkages; (v) the dsRNA comprises at least four 2′-fluoro modifications; (vi) the dsRNA comprises a duplex region of 12-40 nucleotide pairs in length; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 3, 4 or 5 phosphorothioate internucleotide linkages; (v) the dsRNA comprises at least four 2′-fluoro modifications; (vi) the dsRNA comprises a duplex region of 12-40 nucleotide pairs in length; and (vii) the dsRNA has a blunt end at 5′-end of the antisense strand.

In some embodiments, the dsRNA molecule of the disclosure comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA molecule of the disclosure comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT.

Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

It was found that introducing 4′-modified or 5′-modified nucleotide to the 3′-end of a phosphodiester (PO), phosphorothioate (PS), or phosphorodithioate (PS2) linkage of a dinucleotide at any position of single stranded or double stranded oligonucleotide can exert steric effect to the internucleotide linkage and, hence, protecting or stabilizing it against nucleases.

In some embodiments, 5′-modified nucleoside is introduced at the 3′-end of a dinucleotide at any position of single stranded or double stranded siRNA. For instance, a 5′-alkylated nucleoside may be introduced at the 3′-end of a dinucleotide at any position of single stranded or double stranded siRNA. The alkyl group at the 5′ position of the ribose sugar can be racemic or chirally pure R or S isomer. An exemplary 5′-alkylated nucleoside is 5′-methyl nucleoside. The 5′-methyl can be either racemic or chirally pure R or S isomer.

In some embodiments, 4′-modified nucleoside is introduced at the 3′-end of a dinucleotide at any position of single stranded or double stranded siRNA. For instance, a 4′-alkylated nucleoside may be introduced at the 3′-end of a dinucleotide at any position of single stranded or double stranded siRNA. The alkyl group at the 4′ position of the ribose sugar can be racemic or chirally pure R or S isomer. An exemplary 4′-alkylated nucleoside is 4′-methyl nucleoside. The 4′-methyl can be either racemic or chirally pure R or S isomer. Alternatively, a 4′-O-alkylated nucleoside may be introduced at the 3′-end of a dinucleotide at any position of single stranded or double stranded siRNA. The 4′-O-alkyl of the ribose sugar can be racemic or chirally pure R or S isomer. An exemplary 4′-O-alkylated nucleoside is 4′-O-methyl nucleoside. The 4′-O-methyl can be either racemic or chirally pure R or S isomer.

In some embodiments, 5′-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 5′-alkylated nucleoside is 5′-methyl nucleoside. The 5′-methyl can be either racemic or chirally pure R or S isomer.

In some embodiments, 4′-alkylated nucleoside is introduced at any position on the sense strand or antisense strand of a dsRNA, and such modification maintains or improves potency of the dsRNA. The 4′-alkyl can be either racemic or chirally pure R or S isomer. An exemplary 4′-alkylated nucleoside is 4′-methyl nucleoside. The 4′-methyl can be either racemic or chirally pure R or S isomer.

In some embodiments, 4′-O-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 4′-O-alkylated nucleoside is 4′-O-methyl nucleoside. The 4′-O-methyl can be either racemic or chirally pure R or S isomer.

In some embodiments, the dsRNA molecule of the disclosure can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the dsRNA molecule of the disclosure can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC.

Various publications describe multimeric siRNA which can all be used with the dsRNA of the disclosure. Such publications include WO2007/091269, U.S. Pat. No. 7,858,769, WO2010/141511, WO2007/117686, WO2009/014887, and WO2011/031520 which are hereby incorporated by their entirely.

As described in more detail below, the RNAi agent that contains conjugations of one or more carbohydrate moieties to an RNAi agent may improve one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be cyclic group or acyclic group. The cyclic group can be selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalinyl. The acyclic group can be a serinol backbone or diethanolamine backbone.

### IV. Delivery of an RNAi Agent of the Disclosure

The delivery of a RNAi agent of the disclosure to a cell e.g., a skeletal muscle cell, e.g., a skeletal myocyte and/or a cardiomyocyte, e.g., a cell within a subject, such as a human subject (e.g., a subject in need thereof, such as a subject having a target gene-associated disorder, e.g., skeletal muscle and/or cardiac muscle disease or disorder or lung disease or disorder, can be achieved in a number of different ways. For example, delivery may be performed by contacting a cell with an RNAi agent of the disclosure either in vitro or in vivo. In vivo delivery may also be performed directly by administering a composition comprising an RNAi agent, e.g., a dsRNA, to a subject. Alternatively, in vivo delivery may be performed indirectly by administering one or more vectors that encode and direct the expression of the RNAi agent. These alternatives are discussed further below.

In general, any method of delivering a nucleic acid molecule (in vitro or in vivo) can be adapted for use with a RNAi agent of the disclosure (see e.g., Akhtar S. and Julian R L., (1992) Trends Cell. Biol. 2(5):139-144 and WO94/02595, which are incorporated herein by reference in their entireties). For in vivo delivery, factors to consider in order to deliver an RNAi agent include, for example, biological stability of the delivered agent, prevention of non-specific effects, and accumulation of the delivered agent in the target tissue. The non-specific effects of an RNAi agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. For administering a RNAi agent systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the RNAi agent to the target tissue and avoid undesirable off-target effects (e.g., without wishing to be bound by theory, use of GNAs as described herein has been identified to destabilize the seed region of a dsRNA, resulting in enhanced preference of such dsRNAs for on-target effectiveness, relative to off-target effects, as such off-target effects are significantly weakened by such seed region destabilization). Conjugation of an RNAi agent to an aptamer has been shown to inhibit tumor growth and mediate tumor regression in a mouse model of prostate cancer (McNamara, J O. et al., (2006) Nat. Biotechnol. 24:1005-1015). In an alternative embodiment, the RNAi agent can be delivered using drug delivery systems such as a nanoparticle, a dendrimer, a polymer, liposomes, or a cationic delivery system. Positively charged cationic delivery systems facilitate binding of molecule RNAi agent (negatively charged) and also enhance interactions at the negatively charged cell membrane to permit efficient uptake of an RNAi agent by the cell. Cationic lipids, dendrimers, or polymers can either be bound to an RNAi agent, or induced to form a vesicle or micelle (see e.g., Kim S H. et al., (2008) Journal of Controlled Release 129(2):107-116) that encases an RNAi agent. The formation of vesicles or micelles further prevents degradation of the RNAi agent when administered systemically. Methods for making and administering cationic- RNAi agent complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al. (2007) J Hypertens. 25:197-205, which are incorporated herein by reference in their entirety). Some non-limiting examples of drug delivery systems useful for systemic delivery of RNAi agents include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a RNAi agent forms a complex with cyclodextrin for systemic administration. Methods for administration and pharmaceutical compositions of RNAi agents and cyclodextrins can be found in U.S. Pat. No. 7,427,605, which is herein incorporated by reference in its entirety.

Certain aspects of the instant disclosure relate to a method of reducing the expression of a target gene in a skeletal muscle cell and/or a cardiac muscle cell, comprising contacting said cell with the double-stranded RNAi agent of the disclosure.

In certain embodiments, the RNAi agent is taken up on one or more tissue or cell types present in organs, e.g., skeletal muscle tissue and/or cardiac muscle tissue.

Certain aspects of the instant disclosure relate to a method of reducing the expression of a target gene in a lung cell, comprising contacting said cell with the double-stranded RNAi agent of the disclosure.

In certain embodiments, the RNAi agent is taken up on one or more tissue or cell types present in organs, e.g., lung tissue.

Another aspect of the disclosure relates to a method of reducing the expression and/or activity of a target gene in a subject, comprising administering to the subject the double-stranded RNAi agent of the disclosure.

Another aspect of the disclosure relates to a method of treating a subject having a target gene-associated disorder or at risk of having or at risk of developing a target gene-associated disorder, comprising administering to the subject a therapeutically effective amount of the double-stranded RNAi agent of the disclosure, thereby treating the subject.

In one embodiment, the double-stranded RNAi agent is administered subcutaneously.

In one embodiment, the double-stranded RNAi agent is administered intramuscularly.

In one embodiment, the double-stranded RNAi agent is administered by intravenously.

In one embodiment, the double-stranded RNAi agent is administered by pulmonary system administration, e.g., intranasal administration, or oral inhalative administration.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the disclosure. A composition that includes a RNAi agent can be delivered to a subject by a variety of routes. Exemplary routes include pulmonary system, intravenous, subcutaneous, and nasal.

The RNAi agents of the disclosure can be incorporated into pharmaceutical compositions suitable for administration. Such compositions typically include one or more species of RNAi agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present disclosure may be administered in a number of ways depending upon whether local or systemic treatment is desired and upon the area to be treated. Administration may be intratracheal, intranasal, oral, parenteral, or pulmonary, e.g., by inhalation or insufflation of powders or aerosols, including by nebulizer. Parenteral administration includes intravenous drip, subcutaneous, intraperitoneal, or intramuscular injection.

The route and site of administration may be chosen to enhance targeting. For example, to target muscle cells, intramuscular injection into the muscles of interest would be a logical choice. Lung cells might be targeted by administering the RNAi agent in powder or aerosol form. The vascular endothelial cells could be targeted by coating a balloon catheter with the RNAi agent and mechanically introducing the RNA.

Compositions for pulmonary system delivery may include aqueous solutions, e.g., for intranasal or oral inhalative administration, suitable carriers composed of, e.g., lipids (liposomes, niosomes, microemulsions, lipidic micelles, solid lipid nanoparticles) or polymers (polymer micelles, dendrimers, polymeric nanoparticles, nanogels, nanocapsules), adjuvant, e.g., for oral inhalative administration. Aqueous compositions may be sterile and may optionally contain buffers, diluents, absorption enhancers and other suitable additives. Such administration permits both systemic and local delivery of the double stranded RNAi agents of the invention.

Intranasal administration may include instilling or insufflating a double stranded RNAi agent into the nasal cavity with syringes or droppers by applying a few drops at a time or via atomization. Suitable dosage forms for intranasal administration include drops, powders, nebulized mists, and sprays. Nasal delivery devices include, but not limited to, vapor inhaler, nasal dropper, spray bottle, metered dose spray pump, gas driven spray atomizer, nebulizer, mechanical powder sprayer, breath actuated inhaler, and insufflator. Devices for delivery deeper into the respiratory system, e.g., into the lung, include nebulizer, pressured metered-dose inhaler, dry powder inhaler, and thermal vaporization aerosol device. Devices for delivery by inhalation are available from commercial suppliers. Devices can be fixed or variable dose, single or multidose, disposable or reusable depending on, for example, the disease or disorder to be prevented or treated, the volume of the agent to be delivered, the frequency of delivery of the agent, and other considerations in the art.

Oral inhalative administration may include use of device, e.g., a passive breath driven or active power driven single/-multiple dose dry powder inhaler (DPI), to deliver a double stranded RNAi agent to the pulmonary system. Suitable dosage forms for oral inhalative administration include powders and solutions. Suitable devices for oral inhalative administration include nebulizers, metered-dose inhalers, and dry powder inhalers. Dry powder inhalers are of the most popular devices used to deliver drugs, especially proteins to the lungs. Exemplary commercially available dry powder inhalers include Spinhaler (Fisons Pharmaceuticals, Rochester, NY) and Rotahaler (GSK, RTP, NC). Several types of nebulizers are available, namely jet nebulizers, ultrasonic nebulizers, vibrating mesh nebulizers. Jet nebulizers are driven by compressed air. Ultrasonic nebulizers use a piezoelectric transducer in order to create droplets from an open liquid reservoir. Vibrating mesh nebulizers use perforated membranes actuated by an annular piezoelement to vibrate in resonant bending mode. The holes in the membrane have a large cross-section size on the liquid supply side and a narrow cross-section size on the side from where the droplets emerge. Depending on the therapeutic application, the hole sizes and number of holes can be adjusted. Selection of a suitable device depends on parameters, such as nature of the drug and its formulation, the site of action, and pathophysiology of the lung. Aqueous suspensions and solutions are nebulized effectively. Aerosols based on mechanically generated vibration mesh technologies also have been used successfully to deliver proteins to lungs.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves, and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added. Compositions suitable for oral administration of the agents of the invention are further described in PCT Application No. PCT/US20/33156, the entire contents of which are incorporated herein by reference.

Formulations for parenteral administration may include sterile aqueous solutions which may also contain buffers, diluents, and other suitable additives. Intraventricular injection may be facilitated by an intraventricular catheter, for example, attached to a reservoir. For intravenous use, the total concentration of solutes may be controlled to render the preparation isotonic.

In one embodiment, the administration of the siRNA compound, e.g., a double-stranded siRNA compound, is parenteral, e.g., intravenous (e.g., as a bolus or as a diffusible infusion), intradermal, intraperitoneal, intramuscular, subcutaneous, pulmonary system, or intranasal.

Administration can be provided by the subject or by another person, e.g., a health care provider. The medication can be provided in measured doses or in a dispenser which delivers a metered dose. Selected modes of delivery are discussed in more detail below.

### A. Vector Encoded RNAi Agents of the Disclosure

RNAi agents targeting the target gene can be expressed from transcription units inserted into DNA or RNA vectors (see, e.g., Couture, A, et al., TIG. (1996), 12:5-10; WO 00/22113, WO 00/22114, and U.S. Pat. No. 6,054,299). Expression can be sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of a RNAi agent can be transcribed from a promoter on an expression vector. Where two separate strands are to be expressed to generate, for example, a dsRNA, two separate expression vectors can be co-introduced (e.g., by transfection or infection) into a target cell. Alternatively, each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

RNAi agent expression vectors are generally DNA plasmids or viral vectors. Expression vectors compatible with eukaryotic cells, such as those compatible with vertebrate cells, can be used to produce recombinant constructs for the expression of a RNAi agent as described herein. Delivery of RNAi agent expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno- associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of a RNAi agent will generally require regulatory elements, e.g., promoters, enhancers, etc., to ensure the expression of the RNAi agent in target cells. Other aspects to consider for vectors and constructs are known in the art.

### V. Pharmaceutical Compositions of the Invention

The present disclosure also includes pharmaceutical compositions and formulations which include the RNAi agents of the disclosure. In one embodiment, provided herein are pharmaceutical compositions containing an RNAi agent, as described herein, and a pharmaceutically acceptable carrier. The pharmaceutical compositions containing the RNAi agent are useful for treating a skeletal muscle and/or a cardiac muscle disorder, disease or lung disorder or disease, or condition treatable by reduction or inhibition of the expression or activity of a target gene, e.g., a skeletal muscle and/or cardiac muscle disorder or disease or lung disease or disorder.

In some embodiments, the pharmaceutical compositions of the invention are sterile. In another embodiment, the pharmaceutical compositions of the invention are pyrogen free.

Such pharmaceutical compositions are formulated based on the mode of delivery. One example is compositions that are formulated for systemic administration via parenteral delivery, e.g., by intravenous (IV), intramuscular (IM), or for subcutaneous (subQ) delivery.

The pharmaceutical compositions of the disclosure may be administered in dosages sufficient to inhibit expression of a target gene. In general, a suitable dose of an RNAi agent of the disclosure will be in the range of about 0.001 to about 200.0 milligrams per kilogram body weight of the recipient per day, generally in the range of about 1 to 50 mg per kilogram body weight per day.

A repeat-dose regimen may include administration of a therapeutic amount of an RNAi agent on a regular basis, such as monthly to once every six months. In certain embodiments, the RNAi agent is administered about once per quarter (i.e., about once every three months) to about twice per year.

After an initial treatment regimen (e.g., loading dose), the treatments can be administered on a less frequent basis.

In other embodiments, a single dose of the pharmaceutical compositions can be long lasting, such that subsequent doses are administered at not more than 1, 2, 3, or 4 or more month intervals. In some embodiments of the disclosure, a single dose of the pharmaceutical compositions of the disclosure is administered once per month. In other embodiments of the disclosure, a single dose of the pharmaceutical compositions of the disclosure is administered once per quarter to twice per year.

The skilled artisan will appreciate that certain factors can influence the dosage and timing required to effectively treat a subject, including but not limited to the severity of the disease or disorder, previous treatments, the general health or age of the subject, and other diseases present. Moreover, treatment of a subject with a therapeutically effective amount of a composition can include a single treatment or a series of treatments.

Estimates of effective dosages and in vivo half-lives for the iRNAs encompassed by the disclosure can be made using conventional methodologies or on the basis of in vivo testing using a suitable animal model. A suitable animal model, e.g., a mouse or a rat, e.g., an animal containing a transgene expressing a skeletal muscle target gene and/or a cardiac muscle target gene or lung tissue target gene, can be used to determine the therapeutically effective dose and/or an effective dosage regimen for administration of an iRNA agent of the invention.

The pharmaceutical compositions of the present disclosure may be administered in a number of ways depending upon whether local or systemic treatment is desired and upon the area to be treated. Administration may be local (e.g., by intramuscular injection) or parenteral. Parenteral administration includes intravenous, intraarterial, subcutaneous, intraperitoneal or intramuscular injection or infusion; subdermal, e.g., via an implanted device.

Pharmaceutically acceptable organic or inorganic excipients suitable for non-parenteral administration which do not deleteriously react with nucleic acids can also be used to formulate the compositions of the present disclosure. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present disclosure can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings or aromatic substances and the like which do not deleteriously interact with the nucleic acid(s) of the formulation.

Aqueous suspensions can contain substances which increase the viscosity of the suspension including, for example, sodium carboxymethylcellulose, sorbitol or dextran. The suspension can also contain stabilizers.

In some embodiments, pharmaceutical compositions featured in the disclosure include (a) one or more RNAi agents and (b) one or more agents which function by a non-RNAi mechanism and which are useful in treating an muscular or pulmonary disorder or disease, e.g., DMD.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulating a range of dosage for use in humans. The dosage of compositions featured herein in the disclosure lies generally within a range of circulating concentrations that include the ED₅₀ with little or no toxicity. The dosage can vary within this range depending upon the dosage form employed and the route of administration utilized. For any compound used in the methods featured in the disclosure, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the RNAi agents featured in the disclosure can be administered in combination with other known agents effective in treatment of pathological processes mediated by nucleotide repeat expression. In any event, the administering physician can adjust the amount and timing of RNAi agent administration on the basis of results observed using standard measures of efficacy known in the art or described herein.

### VI. Kits

In certain aspects, the instant disclosure provides kits that include a suitable container containing a pharmaceutical formulation of a siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof).

Such kits include one or more dsRNA agent(s) and instructions for use, e.g., instructions for administering a prophylactically or therapeutically effective amount of a dsRNA agent(s) provided herein. The dsRNA agent may be in a vial or a pre-filled syringe. The kits may optionally further comprise means for administering the dsRNA agent (e.g., an injection device, such as a pre-filled syringe ), or means for measuring the inhibition of the target gene (e.g., means for measuring the inhibition of the target gene mRNA, target gene protein, and/or target gene activity). Such means for measuring the inhibition of the target gene may comprise a means for obtaining a sample from a subject, such as, e.g., a plasma sample. The kits of the invention may optionally further comprise means for determining the therapeutically effective or prophylactically effective amount.

In certain embodiments the individual components of the pharmaceutical formulation may be provided in one container. Alternatively, it may be desirable to provide the components of the pharmaceutical formulation separately in two or more containers, e.g., one container for a siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

### X. Methods of the Invention

Another aspect of the invention relates to a method of reducing the expression of a target gene in a cell, comprising contacting the cell with a dsRNA agent of the invention. The methods include contacting the cell with a dsRNA of the disclosure and maintaining the cell for a time sufficient to obtain degradation of the mRNA transcripts of a target gene, thereby inhibiting expression of the target gene in the cell.

Reduction in gene expression can be assessed by any methods known in the art. For example, a reduction in the expression of a target may be determined by determining the mRNA expression level of the target gene using methods routine to one of ordinary skill in the art, e.g., northern blotting, qRT-PCR; by determining the protein level of a target protein using methods routine to one of ordinary skill in the art, such as western blotting, immunological techniques.

In the methods of the disclosure the extrahepatic cell, e.g., muscle cell, e.g., the skeletal muscle cell and/or cardiac muscle cell, or lung cell, may be contacted in vitro or in vivo, i.e., the cell may be within a subject. Contacting a cell in vivo with the RNAi agent includes contacting a cell or group of cells within a subject, e.g., a human subject, with the RNAi agent. Combinations of in vitro and in vivo methods of contacting a cell are also possible.

A cell suitable for treatment using the methods of the disclosure may be any cell that expresses a target gene. A cell suitable for use in the methods of the disclosure may be a mammalian cell, e.g., a primate cell (such as a human cell or a non-human primate cell, e.g., a monkey cell or a chimpanzee cell), a non-primate cell (such as a rat cell, or a mouse cell. In one embodiment, the cell is a human cell, e.g., a human muscle cell or a human lung cell.

Contacting a cell may be direct or indirect, as discussed above. Furthermore, contacting a cell may be accomplished via a targeting ligand, including any ligand described herein or known in the art.

The term “inhibiting,” as used herein, is used interchangeably with “reducing,” “silencing,” “downregulating,” “suppressing” and other similar terms, and includes any level of inhibition. In certain embodiments, a level of inhibition, e.g., for an RNAi agent of the instant disclosure, can be assessed in cell culture conditions, e.g., wherein cells in cell culture are transfected via Lipofectamine™-mediated transfection at a concentration in the vicinity of a cell of 10 nM or less, 1 nM or less, etc. Knockdown of a given RNAi agent can be determined via comparison of pre-treated levels in cell culture versus post-treated levels in cell culture, optionally also comparing against cells treated in parallel with a scrambled or other form of control RNAi agent. Knockdown in cell culture of, e.g., 50% or more, can thereby be identified as indicative of “inhibiting” or “reducing”, “downregulating” or “suppressing”, etc. having occurred. It is expressly contemplated that assessment of targeted mRNA or encoded protein levels (and therefore an extent of “inhibiting”, etc. caused by a RNAi agent of the disclosure) can also be assessed in in vivo systems for the RNAi agents of the instant disclosure, under properly controlled conditions as described in the art.

The phrase “inhibiting expression of a target gene” or “inhibiting expression of a target,” as used herein, includes inhibition of expression of any target gene (such as, e.g., a mouse target gene, a rat target gene, a monkey target gene, or a human target gene) as well as variants or mutants of a target gene that encode a target protein. Thus, the target gene may be a wild-type target gene, a mutant target gene, or a transgenic target gene in the context of a genetically manipulated cell, group of cells, or organism.

“Inhibiting expression of a target gene” includes any level of inhibition of a target gene, e.g., at least partial suppression of the expression of a target gene, such as an inhibition by at least 20%. In certain embodiments, inhibition is by at least 30%, at least 40%, at least 50%, at least about 60%, at least 70%, at least about 80%, at least 85%, at least 90%, at least 95%, at least 96%, at least 97%, at least 98%, or at least 99%; or to below the level of detection of the assay method. In certain method, inhibition is measured at a 10 nM concentration of the siRNA using a luciferase assay.

The expression of a target gene may be assessed based on the level of any variable associated with target gene expression, e.g., target mRNA level or target protein level.

Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the disclosure, expression of a target gene is inhibited by at least 20%, 30%, 40%, 50%, 60%, 70%, 80%, 85%, 90%, or 95%, or to below the level of detection of the assay. In certain embodiments, the methods include a clinically relevant inhibition of expression of a target gene, e.g. as demonstrated by a clinically relevant outcome after treatment of a subject with an agent to reduce the expression of a target gene.

Inhibition of the expression of a target gene may be manifested by a reduction of the amount of mRNA expressed by a first cell or group of cells (such cells may be present, for example, in a sample derived from a subject) in which a target gene is transcribed and which has or have been treated (e.g., by contacting the cell or cells with a RNAi agent of the disclosure, or by administering a RNAi agent of the disclosure to a subject in which the cells are or were present) such that the expression of a target gene is inhibited, as compared to a second cell or group of cells substantially identical to the first cell or group of cells but which has not or have not been so treated (control cell(s) not treated with a RNAi agent or not treated with a RNAi agent targeted to the genome of interest). The degree of inhibition may be expressed in terms of

$$\frac{( mRNA in control cells ) - ( mRNA in treated cells )}{( mRNA in control cells )} \cdot 100 \%$$

In other embodiments, inhibition of the expression of a target gene may be assessed in terms of a reduction of a parameter that is functionally linked to a target gene expression, e.g., target protein expression. Target gene silencing may be determined in any cell expressing a target gene, either endogenous or heterologous from an expression construct, and by any assay known in the art.

Inhibition of the expression of a target protein may be manifested by a reduction in the level of the target protein that is expressed by a cell or group of cells (e.g., the level of protein expressed in a sample derived from a subject). As explained above, for the assessment of genome suppression, the inhibition of protein expression levels in a treated cell or group of cells may similarly be expressed as a percentage of the level of protein in a control cell or group of cells.

A control cell or group of cells that may be used to assess the inhibition of the expression of a target gene includes a cell or group of cells that has not yet been contacted with an RNAi agent of the disclosure. For example, the control cell or group of cells may be derived from an individual subject (e.g., a human or animal subject) prior to treatment of the subject with an RNAi agent.

The level of target gene mRNA that is expressed by a cell or group of cells may be determined using any method known in the art for assessing RNA expression. In one embodiment, the level of expression of target gene in a sample is determined by detecting a transcribed polynucleotide, or portion thereof, e.g., mRNA of the target gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating target mRNA may be detected using methods the described in WO2012/177906, the entire contents of which are hereby incorporated herein by reference.

In some embodiments, the level of expression of target gene is determined using a nucleic acid probe. The term “probe”, as used herein, refers to any molecule that is capable of selectively binding to a specific target nucleic acid or protein, or fragment thereof. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of RNA levels involves contacting the isolated RNA with a nucleic acid molecule (probe) that can hybridize to target RNA. In one embodiment, the RNA is immobilized on a solid surface and contacted with a probe, for example by running the isolated RNA on an agarose gel and transferring the RNA from the gel to a membrane, such as nitrocellulose. In an alternative embodiment, the probe(s) are immobilized on a solid surface and the RNA is contacted with the probe(s), for example, in an Affymetrix® gene chip array. A skilled artisan can readily adapt known RNA detection methods for use in determining the level of target mRNA.

An alternative method for determining the level of expression of target in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of target is determined by quantitative fluorogenic RT-PCR (i.e., the TaqMan™ System), by a Dual-Glo® Luciferase assay, or by other art-recognized method for measurement of target expression or mRNA level.

The expression level of target mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of target expression level may also comprise using nucleic acid probes in solution.

In some embodiments, the level of RNA expression is assessed using branched DNA (bDNA) assays or real time PCR (qPCR). The use of this PCR method is described and exemplified in the Examples presented herein. Such methods can also be used for the detection of target nucleic acids.

The level of target protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of target proteins.

In some embodiments, the efficacy of the methods of the disclosure in the treatment of a target gene-related disease is assessed by a decrease in target mRNA level (e.g, by assessment of a blood target gene level, or otherwise).

In some embodiments, the efficacy of the methods of the disclosure in the treatment of a target gene-related disease is assessed by a decrease in target mRNA level (e.g, by assessment of a muscle or lung sample for target level, by biopsy, or otherwise). In some embodiments of the methods of the disclosure, the RNAi agent is administered to a subject such that the RNAi agent is delivered to a specific site within the subject. The inhibition of expression of target may be assessed using measurements of the level or change in the level of target mRNA or target protein in a sample derived from a specific site within the subject, e.g., muscle or lung cells. In certain embodiments, the methods include a clinically relevant inhibition of expression of target, e.g. as demonstrated by a clinically relevant outcome after treatment of a subject with an agent to reduce the expression of target gene.

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

The in vivo methods of the disclosure may include administering to a subject a composition containing a RNAi agent, where the RNAi agent includes a nucleotide sequence that is complementary to at least a part of an RNA transcript of the target gene of the subject to be treated. When the organism to be treated is a mammal such as a human, the composition can be administered by means including, but not limited to intraperitoneal or parenteral routes, including intravenous, intramuscular, subcutaneous, transdermal, airway (aerosol), nasal, administration. In certain embodiments, the compositions are administered by intravenous infusion or injection. In certain embodiments, the compositions are administered by subcutaneous injection. In certain embodiments, the compositions are administered by pulmonary delivery, e.g., oral inhalation or intranasal delivery.

In some embodiments, the administration is via a depot injection. A depot injection may release the RNAi agent in a consistent way over a prolonged time period. Thus, a depot injection may reduce the frequency of dosing needed to obtain a desired effect, e.g., a desired inhibition of the target gene, or a therapeutic or prophylactic effect. A depot injection may also provide more consistent serum concentrations. Depot injections may include subcutaneous injections or intramuscular injections. In certain embodiments, the depot injection is a subcutaneous injection.

In one embodiment, the double-stranded RNAi agent is administered by pulmonary system administration, e.g., intranasal administration or oral inhalative administration. Pulmonary system administration may be via a syringe, a dropper, atomization, or use of device, e.g., a passive breath driven or active power driven single/-multiple dose dry powder inhaler (DPI) device.

The mode of administration may be chosen based upon whether local or systemic treatment is desired and based upon the area to be treated. The route and site of administration may be chosen to enhance targeting.

In one aspect, the present disclosure also provides methods for inhibiting the expression of a target gene in a mammal. The methods include administering to the mammal a composition comprising a dsRNA that targets a target gene in a cell of the mammal and maintaining the mammal for a time sufficient to obtain degradation of the RNA transcript of the target gene, thereby inhibiting expression of the target gene in the cell. Reduction in genome expression can be assessed by any methods known it the art and by methods, e.g. qRT-PCR, described herein. Reduction in protein production can be assessed by any methods known it the art and by methods, e.g. ELISA, described herein.

The present disclosure further provides methods of treatment of a subject in need thereof. The treatment methods of the disclosure include administering an RNAi agent of the disclosure to a subject, e.g., a subject that would benefit from inhibition of target gene expression, in a therapeutically effective amount of a RNAi agent targeting a target gene or a pharmaceutical composition comprising a RNAi agent targeting a target gene.

Target genes (described above), target gene-associated disorders, and subjects that would benefit from a reduction or inhibition of target gene expression, e.g., those having a target gene-associated disease, subjects at risk of developing a target gene-associate disease, are described below.

An RNAi agent of the disclosure may be administered as a “free RNAi agent.” A free RNAi agent is administered in the absence of a pharmaceutical composition. The naked RNAi agent may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the RNAi agent can be adjusted such that it is suitable for administering to a subject. In certain embodiments, the free RNAi agent may be formulated in water or normal saline.

Alternatively, an RNAi agent of the disclosure may be administered as a pharmaceutical composition, such as a dsRNA liposomal formulation.

In one aspect, the present invention provides a method of treating a subject having a muscle disorder, e.g., a skeletal muscle disorder and/or a cardiac muscle disorder, comprising administering to the subject a therapeutically effective amount of a dsRNA agent of the invention, thereby treating the subject.

Exemplary muscle disorders include Myostatin-related muscle hypertrophy, congenital myasthenic syndrome, facioscapulohumeral muscular dystrophy (FSHD), Spinal Muscular Atrophy (SMA), Myotonic Dystrophy Type 1 (DM1), Pompe disease, PLN cardiomyopathy, spasticity, obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); hypertrophic cardiomyopathy (HCM); and PLN cardiomyopathy.

Exemplary cardiac muscle disorders include obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); hypertrophic cardiomyopathy (HCM); and PLN cardiomyopathy.

Exemplary skeletal muscle disorders include Myostatin-related muscle hypertrophy, congenital myasthenic syndrome, facioscapulohumeral muscular dystrophy (FSHD), Spinal Muscular Atrophy (SMA), Myotonic Dystrophy Type 1 (DM1), Pompe disease, PLN cardiomyopathy, and spasticity.

In one aspect, the present invention provides a method of treating a subject having a lung disorder, comprising administering to the subject a therapeutically effective amount of a dsRNA agent of the invention, thereby treating the subject.

Exemplary lung disorders include pulmonary fibrosis e.g. idiopathic pulmonary fibrosis, non-specific interstitial pneumonia (NSIP), usual interstitial pneumonia (UIP), Hermansky-Pudlak syndrome, progressive massive fibrosis (a complication of coal workers' pneumoconiosis), connective tissue disease-related pulmonary fibrosis, airway fibrosis in asthma and COPD, acute respiratory distress syndrome (ARDS) associated fibrosis, acute lung injury; radiation-induced fibrosis; familial pulmonary fibrosis; pulmonary hypertension, asthma, asthma and chronic rhinosinusitis, and nasal polyps and chronic rhinosinusitis.

The dsRNA agent of the invention can be delivered to a subject by a variety of routes, depending on the type of genes targeted and the type of disorders to be treated. In some embodiments, the dsRNA agent is administered extra-hepatically, such as intravenous, intramuscular, or subcutaneous administration.

The disclosure further provides methods for the use of a RNAi agent or a pharmaceutical composition thereof, e.g., for treating a subject that would benefit from reduction or inhibition of target gene expression, e.g., a subject having a target-gene-associated disorder, in combination with other pharmaceuticals or other therapeutic methods, e.g., with known pharmaceuticals or known therapeutic methods, such as, for example, those which are currently employed for treating these disorders.

Examples of the additional therapeutic agents which can be used with an RNAi agent of the invention include, but are not limited to, cardiovascular diseases-treating agents, anti-hyperlipemic agents, hypotensive or antihypertensive agents, chemotherapeutic agents, immunotherapeutic agents, immunosuppressive agents, nonsteroidal anti-inflammatory drugs (NSAIDs), colchicine, corticosteroids, and the like. Such combination therapies may advantageously utilize lower dosages of the administered therapeutic agents, thus avoiding possible toxicities or complications associated with the various monotherapies.

Anti-hyperlipemic agents include, for example, statin-based compounds which are cholesterol synthesis inhibitors (e.g., pravastatin, simvastatin, lovastatin, atorvastatin, fluvastatin, rosuvastatin and the like), squalene synthetase inhibitors or fibrate compounds having a triglyceride-lowering effect (e.g., fenofibrate, gemfibrozil, bezafibrate, clofibrate, sinfibrate, clinofibrate and the like), niacin, PCSK9 inhibitors, triglyceride lowing agents or cholesterol sequesting agents.

Hypotensive agents include, for example, angiotensin converting enzyme inhibitors (e.g., captopril, enalapril, delapril, benazepril, cilazapril, enalapril, enalaprilat, fosinopril, lisinopril, moexipril, perindopril, quinapril, ramipril, trandolapril and the like) or angiotensin II antagonists (e.g., losartan, candesartan cilexetil, olmesartan medoxomil, eprosartan, valsartan, telmisartan, irbesartan, tasosartan, pomisartan, ripisartan forasartan, and the like) or calcium channel blockers (e.g., amlodipine) or aspirin.

In addition, agents whose cachexia improving effect has been established in an animal model or at a clinical stage, such as cyclooxygenase inhibitors (e.g., indomethacin and the like), progesterone derivatives (e.g., megestrol acetate), glucosteroid (e.g., dexamethasone and the like), metoclopramide-based agents, tetrahydrocannabinol-based agents, lipid metabolism improving agents (e.g., eicosapentanoic acid and the like), growth hormones, IGF-1, antibodies against TNF-α, LIF, IL-6 and oncostatin M may also be employed concomitantly with an RNAi agent according to the present invention. Additional therapeutic agents for use in the treatment of diseases or conditions related to metabolic disorders and/or impaired neurological signaling would be apparent to the skilled artisan and are within the scope of this disclosure.

The RNAi agent and additional therapeutic agents may be administered at the same time or in the same combination, or the additional therapeutic agent can be administered as part of a separate composition or at separate times or by another method known in the art or described herein.

In one embodiment, the method includes administering a composition featured herein such that expression of the target gene is decreased, for at least one month. In some embodiments, expression is decreased for at least 2 months, 3 months, or 6 months.

In certain embodiments, administration includes a loading dose administered at a higher frequency, e.g., once per day, twice per week, once per week, for an initial dosing period, e.g., 2-4 doses.

In some embodiments, the RNAi agents useful for the methods and compositions featured herein specifically target RNAs (primary or processed) of the target gene. Compositions and methods for inhibiting the expression of these genes using RNAi agents can be prepared and performed as described herein.

Administration of the dsRNA according to the methods of the disclosure may result in a reduction of the severity, signs, symptoms, or markers of such diseases or disorders in a patient with a target gene-associated disorder. By “reduction” in this context is meant a statistically significant or clinically significant decrease in such level. The reduction can be, for example, at least 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. It is well within the ability of one skilled in the art to monitor efficacy of treatment or prevention by measuring any one of such parameters, or any combination of parameters. In connection with the administration of a RNAi agent targeting a target gene of interest or pharmaceutical composition thereof, “effective against” a target gene-associated disorder indicates that administration in a clinically appropriate manner results in a beneficial effect for at least a statistically significant fraction of patients, such as an improvement of symptoms, a cure, a reduction in disease, extension of life, improvement in quality of life, or other effect generally recognized as positive by medical doctors familiar with treating target gene-associated disorders and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and at least 20%, 30%, 40%, 50%, or more can be indicative of effective treatment. Efficacy for a given RNAi agent drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using a RNAi agent or RNAi agent formulation as described herein.

Subjects can be administered a therapeutic amount of dsRNA, such as about 0.01 mg/kg to about 200 mg/kg.

The RNAi agent can be administered over a period of time, on a regular basis. In certain embodiments, after an initial treatment regimen, the treatments can be administered on a less frequent basis. Administration of the RNAi agent can reduce target gene levels, e.g., in a cell, tissue, blood sample or other compartment of the patient by at least 20%, 30%, 40%, 50%, 55%, 60%, 65%, 70,% 75%, 80%, 85%, 90%, 95%, 96%, 97%, 98%, or at least about 99% or more. In one embodiment, administration of the RNAi agent can reduce target gene levels, e.g., in a cell, tissue, blood sample, or other compartment of the patient by at least 50%.

Before administration of a full dose of the RNAi agent, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Alternatively, the RNAi agent can be administered by oral administration, pulmonary administration, intravenously, i.e., by intravenous injection, or subcutaneously, i.e., by subcutaneous injection. One or more injections may be used to deliver the desired, e.g., monthly dose of RNAi agent to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of RNAi agent on a regular basis, such as monthly or extending to once a quarter, twice per year, once per year. In certain embodiments, the RNAi agent is administered about once per month to about once per quarter (i.e., about once every three months).

### XI. Target Genes and Target-Gene-Associated Diseases

Specific exemplary target genes that mediate a muscle disorder, e.g., a skeletal muscle disorder and/or cardiac muscle disorder, include, but are not limited to, adrenoceptor beta 1 (ADRB1); calcium voltage-gated channel subunit alpha1 C (CACNA1C); calcium voltage-gated channel subunit alpha1 G (CACNA1G) (T type calcium cchannel); angiotensin II receptor type 1 (AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); or Phosphodiesterase 1 (PDE1), myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); Double Homeobox 4 (DUX4), dystrophy myotonic protein kinase (DMPK), glycogen synthase 1 (GYS1), survival of motor neuron 1 (SMN1), and alpha-glucosidase (GAA).

Exemplary target genes that mediate a skeletal muscle disorder include, but are not limited to, myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); and Double Homeobox 4 (DUX4), dystrophy myotonic protein kinase (DMPK), glycogen synthase 1 (GYS1), survival of motor neuron 1 (SMN1), and alpha-glucosidase (GAA).

Exemplary target genes that mediate a cardiac muscle disorder include, but are not limited to, adrenoceptor beta 1 (ADRB1); calcium voltage-gated channel subunit alpha1 C (CACNA1C); calcium voltage-gated channel subunit alpha1 G (CACNA1G) (T type calcium cchannel); angiotensin II receptor type 1 (AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); or Phosphodiesterase 1 (PDE1).

Exemplary target genes that mediate a lung disorder include, but are not limited to, MUC5B, TSLP, IL33, ALOX15, AGER(RAGE), MUC5AC, and STAT6.

In some embodiments, the present invention provides a double-stranded iRNA agent that targets ADRB1 for the treatment of obstructive hypertrophic cardiomyopathy (HOCM); familial hypertrophic cardiomyopathy (FHC); heart failure with preserved ejection fraction (HF-pEF); atrial fibrillation (AFIB); ventricular fibrillation (VFIB); angina; myocardial infarction (MI); and/or heart failure or heart failure with reduced ejection fraction (HFREF).

In some embodiments, the present invention provides a double-stranded iRNA agent that targets CACNA1C for the treatment of supraventricular tachycardia (SVT); AFIB; Angina; and/or HOCM.

In some embodiments, the present invention provides a double-stranded iRNA agent that targets CACNA1G for the treatment of supraventricular tachycardia (SVT); and/or angina.

In some embodiments, the present invention provides a double-stranded iRNA agent that targets AGTR1 for the treatment of HOCM; hypertrophic cardiomyopathy (HCM); and/or HF-pEF. In some embodiments, the present invention provides a double-stranded iRNA agent that targets SCN2A for the prevention and/or treatment of AFIB.

In some embodiments, the present invention provides a double-stranded iRNA agent that targets HCN1 for the prevention and/or treatment of AFIB; treatment, e.g., rate control, in HOCM.

In some embodiments, the present invention provides a double-stranded iRNA agent that targets HCN4 for the prevention and/or treatment of AFIB; treatment, e.g., rate control, in HOCM.

In some embodiments, the present invention provides a double-stranded iRNA agent that targets HCN3 for the prevention and/or treatment of AFIB; treatment, e.g., rate control, in HOCM.

In some embodiments, the present invention provides a double-stranded iRNA agent that targets KCNA5 for the prevention and/or treatment of AFIB, e.g., AFIB in congestive heart failure (CHF).

In some embodiments, the present invention provides a double-stranded iRNA agent that targets KCNJ3 for the prevention and/or treatment of AFIB.

In some embodiments, the present invention provides a double-stranded iRNA agent that targets KCNJ4 for the prevention and/or treatment of AFIB.

In some embodiments, the present invention provides a double-stranded iRNA agent that targets CAMK2D for the prevention and/or treatment of heart failure and/or AFIB.

In some embodiments, the present invention provides a double-stranded iRNA agent that targets PLN for the prevention and/or treatment of HF-rEF, arrhythmia, and/or cardiomyopathy. In some embodiments, the present invention provides a double-stranded iRNA agent that targets PDE1 for the prevention and/or treatment of CHF and/or HF-pEF.

In some embodiment, the present invention provides a double-stranded iRNA agent that targets myostatin for the treatment of Myostatin-related muscle dystrophy.

In some embodiments, the present invention provides a double-stranded iRNA agent that targets CHRNA1 for the treatment of congenital myasthenic syndrome (CMS).

In some embodiments, the present invention provides a double-stranded iRNA agent that targets CHRNB1 for the treatment of congenital myasthenic syndrome (CMS).

In some embodiments, the present invention provides a double-stranded iRNA agent that targets CHRBD for the treatment of congenital myasthenic syndrome (CMS).

In some embodiments, the present invention provides a double-stranded iRNA agent that targets CHRNE for the treatment of congenital myasthenic syndrome (CMS).

In some embodiments, the present invention provides a double-stranded iRNA agent that targets CHRNG for the treatment of congenital myasthenic syndrome (CMS).

In some embodiments, the present invention provides a double-stranded iRNA agent that targets COL 13A1 for the treatment of congenital myasthenic syndrome (CMS).

In some embodiments, the present invention provides a double-stranded iRNA agent that targets LRP4 for the treatment of congenital myasthenic syndrome (CMS).

In some embodiments, the present invention provides a double-stranded iRNA agent that targets MUSK for the treatment of congenital myasthenic syndrome (CMS).

In some embodiments, the present invention provides a double-stranded iRNA agent that targets RAPSN for the treatment of congenital myasthenic syndrome.

In some embodiments, the present invention provides a double-stranded iRNA agent that targets SCN4A for the treatment of congenital myasthenic syndrome (CMS).

In some embodiments, the present invention provides a double-stranded iRNA agent that targets DOK7 for the treatment of congenital myasthenic syndrome (CMS).

In some embodiments, the present invention provides a double-stranded iRNA agent that targets DUX4 for the treatment of Facioscapulohumeral muscular dystrophy (FSHD).

In some embodiments, the present invention provides a double-stranded iRNA agent that targets DMPK for the treatment of myotonic dystrophy.

In some embodiments, the present invention provides a double-stranded iRNA agent that targets GYS1 for the treatment of glycogen storage disease.

In some embodiments, the present invention provides a double-stranded iRNA agent that targets SMN1 for the treatment of spinal muscular atrophy.

In some embodiments, the present invention provides a double-stranded iRNA agent that targets GAA for the treatment of Pompe disease.

In some embodiments, the present invention provides a double-stranded iRNA agent that targets ADRB1 for the treatment of obstructive hypertrophic cardiomyopathy (HOCM); familial hypertrophic cardiomyopathy (FHC); heart failure with preserved ejection fraction (HF-pEF); atrial fibrillation (AFIB); ventricular fibrillation (VFIB); angina; myocardial infarction (MI); and/or heart failure or heart failure with reduced ejection fraction (HFREF).

In some embodiments, the present invention provides a double-stranded iRNA agent that targets MUC5B for the treatment of idiopathic pulmonary fibrosis.

In some embodiments, the present invention provides a double-stranded iRNA agent that targets TSLP for the treatment of asthma.

In some embodiments, the present invention provides a double-stranded iRNA agent that targets IL33 for the treatment of asthma and chronic rhinosinusitis.

In some embodiments, the present invention provides a double-stranded iRNA agent that targets ALOX15 for the treatment of nasal polyps and chronic rhinosinusitis.

In some embodiments, the present invention provides a double-stranded iRNA agent that targets STAT6 for the treatment of pulmonary fibrosis or asthma.

Targeting ADRB1 for the Prevention and or Treatment of HOCM, FHC, HFPEF, AFIB, VFIB, Angina, MI, and/or HFREF

The beta-adrenergic receptors (ADRBs) are part of a family of membrane proteins known as G-protein coupled receptors where, upon binding of a catecholamine to the receptor, stimulates a conformational change in the ADRB that causes coupling with G-proteins. G-proteins consist of a, p, and y subunits and ADRB coupling leads to the dissociation of the G-protein into active Ga and GD subunits to mediate downstream signaling.

Beta-adrenergic receptors (ADRBs) play an important role in the extrinsic control of cardiac contractility and function, and are important drug targets for cardiovascular conditions such as hypertension and congestive heart failure. Inhaled beta-receptor (e.g. “beta-blockers”) remain among the mostly commonly prescribed medications in adults to treat cardiovascular disease.

There are three subtypes of ADRBs (ADRB1, ADRB2 and ADRB3). ADRB1s are the predominant subtype expressed in the heart. Multiple ADRB1 have been described and found to be associated with various cardiovascular phenotypes, such as hypertension, heart failure, higher heart rates, or response to beta-blocker therapy. Genetic variants of the ADRB1 have also been shown to modulate the cardiac responses to catecholamine binding. In addition, ADRB1 signaling has also been shown to play an important role in heart failure (HF), where beta-blocking medications are widely used therapeutic agents. Deleterious effects of ADRB1 signaling include apoptosis, myocyte growth, fibroblast hyperplasia, myopathy, fetal gene induction and proarrhythmia (Mann D L, et al. Circulation. 1992; 85(2):790-804). As an adaptive mechanism in HF, cardiac ADRB1s become less responsive, either downregulating or uncoupling from the G protein pathway (Bristow M R, et al. N Engl J Med. 1982; 307(4):205-211). With respect to atrial fibrillation, ADRB1 variant carriers have been reported to have an increased risk of atrial fibrillation, and higher heart rates during atrial fibrillation. ADRB1 polymorphisms are also associated with ventricular fibrillation (VF) in the context of myocardial infarction (MI).

### Targeting CACNA1C for the Prevention and or Treatment of SVT, AFIB, Angina, and or HOCM

Supraventricular tachycardia (SVT) is a heterogeneous category of cardiac arrhythmias characterized by a fast or tachycardiac rhythm that originates above the atrioventricular (AV) node. The prevalence of SVT is 2.25/1000 persons with a female predominance of 2:1 across all age groups (Lee K W, et al., Curr Probl Cardiol 2008; 33:467-546). The most common SVTs include atrioventricular nodal re-entrant tachycardia, atrioventricular re-entrant tachycardia and atrial tachycardia. SVT increases patient morbidity, particularly when symptoms are frequent or incessant, and in a small cohort of patients with atrial fibrillation (AF) and ventricular pre-excitation, it can be life-threatening.

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78.). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8.). This can cause detrimental symptoms, impair functional status and reduce the quality of life.

Angina represents the most common symptom of ischaemic heart disease, which is a major cause of death and disability worldwide. Nearly 10 million US adults experience stable angina, which occurs when myocardial oxygen supply does not meet demand, resulting in myocardial ischemia. Stable angina is associated with an average annual risk of 3% to 4% for myocardial infarction or death.

Hypertrophic cardiomyopathy is another common heart disorder, usually genetic in origin, that may affect up to 600,000 people in the United States. The disorder, which is characterized by left ventricular hypertrophy, is usually not progressive, but a small subset of patients develop serious complications, such as progressive heart failure, atrial fibrillation, and sudden cardiac death.

Anti-arrhythmic drug therapies, such as calcium channel blockers, are commonly used to treat heart disorders, such as SVT, AFIB, angina and HOCM, by regulating heart function through shaping the action potential and maintaining the rhythm of cardiac contraction.

In particular, calcium channels, such as CACNA1C, play an important role in regulating heart function. CACNA1C encodes for the α-subunit of the CaV1.2 L-type calcium channel (LTCC), which is critical for the plateau phase of the cardiac action potential, cellular excitability, excitation-contraction coupling, and regulation of gene expression. The currently available calcium channel blockers (e.g., dihydropyridines, phenylalkylamines, and benzothiazepines) all act by binding to different sites on CACNA1C and blocking the calcium current.

The CACNA1C calcium channels open and close at specific times to control the flow of calcium ions into cardiomyocytes at each heartbeat. How long the channels are open and closed is regulated to maintain normal heart function. Perturbations of CACNA1C change the structure of calcium channels throughout the body and have been associated with several different cardiac arrhythmia disorders. The altered channels stay open much longer than usual, which allows calcium ions to continue flowing into cells abnormally. The resulting overload of calcium ions within cardiac muscle cells changes the way the heart beats and can cause abnormal heart muscle contraction and arrhythmia.

Increased susceptibility for arrhythmia was observed in patients with gain of function CACNA1C variants under certain conditions (PLoS One. 2014; 9(9): e106982; Splawski I, et al. Cell. 2004 Oct. 1; 119(1):19-31). Specifically, gain of function mutations of CACNA1C revealed a marked reduction in voltage-dependent inactivation. The consequent increase in calcium influx prolongs the cardiac action potential, and thus the QT interval, and can generate early afterdepolarizations capable of triggering cardiac arrhythmias, such as supraventricular tachycardia and atrial fibrillation. Other CACNA1C variants were also shown to be associated with hypertrophic cardiomyopathy, congenital heart defects, and sudden cardiac death.

### Targeting CACNA1G for the Prevention and or Treatment of SVT and or Angina

Supraventricular tachycardia (SVT) is a heterogeneous category of cardiac arrhythmias characterized by a fast or tachycardiac rhythm that originates above the atrioventricular (AV) node. The prevalence of SVT is 2.25/1000 persons with a female predominance of 2:1 across all age groups (Lee K W, et al., Curr Probl Cardiol 2008; 33:467-546). The most common SVTs include atrioventricular nodal re-entrant tachycardia, atrioventricular re-entrant tachycardia and atrial tachycardia. SVT increases patient morbidity, particularly when symptoms are frequent or incessant, and in a small cohort of patients with atrial fibrillation (AF) and ventricular pre-excitation, it can be life-threatening.

Angina represents the most common symptom of ischaemic heart disease, which is a major cause of death and disability worldwide. Nearly 10 million US adults experience stable angina, which occurs when myocardial oxygen supply does not meet demand, resulting in myocardial ischemia. Stable angina is associated with an average annual risk of 3% to 4% for myocardial infarction or death.

Anti-arrhythmic drug therapies, such as calcium channel blockers, are commonly used to treat heart diseases such as SVT and angina by regulating adult heart function through shaping the action potential and maintaining the rhythm of cardiac contraction.

CACNA1G encodes for the subunit of the CaV3,1 T-type calcium channel, which plays a role in the human sinoatrial node and the conduction system. These channels contribute to the heartbeat by influencing pacemaking and the atrioventricular node. Inactivation of CACNA1G significantly slowed the intrinsic in vivo heart rate, prolonged the sinoatrial node recovery time, and slowed pacemaker activity of individual sinoatrial node cells through a reduction of the slope of the diastolic depolarization (Mangoni, et al., Circulation Research 2006, 1422-1430). Thus, selective blockers of CaV3.1 channels hold promise for the therapeutic management of the cardiac diseases that require moderate heart rate reduction, such as SVT. The T-type calcium channels also constitute a promising pharmacological target for the treatment of human diseases, such as epilepsy and chronic pain (Birch P J, et al. Drug Discov Today. 2004; 9:410-418).

### Targeting AGTR1 for the Prevention and or Treatment of HOCM, HCM, and or HFpEF

Hypertrophic cardiomyopathy (HCM) is the most common inheritable cardiac disorder with a phenotypic prevalence of 1:500. It is defined by the presence of left ventricular hypertrophy (LVH) in the absence of loading conditions (hypertension, valve disease) sufficient to cause the observed abnormality.

The obstructive HCM (hypertrophic obstructive cardiomyopathy or HOCM) is subtype of HCM. In HOCM, the wall (septum) between the bottom chambers of the heart thickens. The walls of the pumping chamber can also become stiff. The thickened septum may cause a narrowing that can block or reduce the blood flow from the left ventricle to the aorta, which is a condition called “outflow tract obstruction.”

Increased blood pressure causes a concentric pattern of LVH, which may progress to ventricular dilation and heart failure with preserved ejection fraction (HFpEF). Heart failure with preserved ejection fraction (HFpEF) is a clinical syndrome in which patients have symptoms and signs of heart failure as the result of high ventricular filling pressure despite normal or near normal left ventricular ejection fraction (LVEF≥50 percent). At a cellular level, cardiac myocytes in patients with HFpEF are thicker and shorter than normal myocytes, and collagen content is increased. At the organ level, affected individuals may have concentric remodeling with or without hypertrophy. Increases in myocyte stiffness are mediated in part by relative hypophosphorylation of the sarcomeric molecule titin, due to cyclic guanosine monophosphate (cGMP) deficiency thought to arise primarily as a consequence of increased nitroso-oxidative stress induced by comorbid conditions such as obesity, metabolic syndrome and aging. Cellular and tissue characteristics may become more pronounced as the disease progresses.

Genetic variants in the renin-angiotensin-aldosterone system (RAAS) are considered candidates for these modifying effects. The RAAS system contributes to LVH through effects mediated by circulating angiotensin as well as local activation of RAAS in the myocardium.

Angiotensin (Ang) I, produced from angiotensinogen (AGT), is converted to Ang II predominantly by angiotensin-converting enzyme (ACE) and possibly by chymase 1 (CMA1). Ang II binds primarily to the Ang II type 1 receptor (AGTR1) to promote cell growth and hypertrophy. It also stimulates aldosterone by aldosterone synthase (CYP11B2) synthesis, thereby increasing the release of aldosterone, which promotes fluid retention and cardiac fibrosis.

Previous studies suggested a role for specific genetic variants in genes encoding components of the RAAS pathway in modulation of the severity of LVH in patients with HCM (Orenes-Pifiero E, et al. J Renin Angiotensin Aldosterone Syst 2011; 12: 521-530; Ortlepp J R, et al., Heart 2002; 87: 270-275). In particular, a specific A>C polymorphism of the AGTR1 gene was considered as the pro-LVH allele. Carriers that harbor the pro-LVH allele had greater left ventricular muscle mass and interventricular septum thickness compared to those without the pro-LVH allele (Kolder et al. Eur J Hum Genet. 2012 October; 20(10): 1071-1077). Additional studies further demonstrated that left ventricular mass is associated with the AGTR1 polymorphism, and cardiac hypertrophy was improved by down-regulating AGTR1 (Y. Yang, et al. Exp. Ther. Med., 12 (3) (2016), pp. 1556-1562).

### Targeting SCN2A for the Prevention and or Treatment of AFIB.

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78.). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8.). This can cause detrimental symptoms, impair functional status and reduce the quality of life.

Atrial fibrillation can cause syncope or a temporary loss of consciousness caused by a fall in blood pressure. There is also a possibility of atrial fibrillation developing secondary to an epileptic seizure in cases of atrial fibrillation and transient loss of consciousness. Epileptic seizures are often associated with changes in cardiac autonomic function.

Sodium voltage-gated channel alpha subunit 2 (SCN2A) is one of the genes most commonly associated with early-onset epilepsy, and has recently been linked to autism spectrum disorder and developmental delay. SCN2A encodes the Nav1.2 subunit of voltage-gated sodium channel in neurons, which is important for action potential initiation and conduction. SCN2A gain-of-function mutations have been identified, and the phenotypes range from benign neonatal or infantile seizures to severe epileptic encephalopathy. SCN2A gene deletion acts as protective genetic modifier of sudden unexpected death in epilepsy (SUDEP) and suggest measures of brain-heart association as potential indices of SUDEP susceptibility (V Mishra et al., Hum Mol Genet. 2017 Jun. 1; 26(11):2091-2103). In addition to epilepsy and developmental delays, other manifestations of SCN2A deletion can include movement disorders such as dystonia, abnormal gait, ADHD, autism, dysautonomia (i.e. problems with heart rate, blood pressure, and temperature regulation), and GI problems such as feeding difficulties or reflux.

### Targeting HCN1 for the Prevention and or Treatment of AFIB; Treatment, e.g., Rate Control, in HOCM

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78.). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8.). This can cause detrimental symptoms, impair functional status and reduce the quality of life.

Hypertrophic cardiomyopathy is another common heart disorder, usually genetic in origin, that may affect up to 600,000 people in the United States. The disorder, which is characterized by left ventricular hypertrophy, is usually not progressive, but a small subset of patients develop serious complications, such as progressive heart failure, atrial fibrillation, and sudden cardiac death.

Hyperpolarization-activated cyclic nucleotide-gated (HCN) channels are voltage-gated channels encoded by the HCN1-4 gene family. These channels are primarily expressed in the heart and in the central and peripheral nervous systems. HCN channels conduct K+ and Na+ ions at a ratio of 3:1 to 5:1. They are activated by hyperpolarization of membrane voltage to −50 mV or below, and conduct the hyperpolarization-activated current, termed I_{f} in heart. In the sino-atrial node (SAN), I_{f} plays an essential role in setting the heart rate and mediating its autonomic control.

HCN1 is highly expressed in the SAN and, in addition, non-pacemaking atrial and ventricular cardiomyocytes also express HCN channels, with an increase in I_{f} activity in ventricular myocytes reported in hypertrophy, ischemic cardiomyopathy and heart failure due to re-expression of HCN genes. Studies have shown that I_{f} current density and occurrence is significantly greater in hypertrophic cardiomyocytes and end-stage failing hearts and this is directly related to the arrhythmias

Genetic variants in HCN channels are linked to sinus node dysfunction, atrial fibrillation, ventricular tachycardia, atrio-ventricular block, Brugada syndrome, sudden infant death syndrome, and sudden unexpected death in epilepsy. HCN1 deficient mice display congenital sinus node dysfunction with severely reduced cardiac output.

Several HCN channel blockers including ZD7288, zatebradine, cilobradine and ivabradine are available. The first clinically approved substance from this new class of drugs is ivabradine.

### Targeting HCN4 for the Prevention and or Treatment of AFIB; Treatment, e.g., Rate Control, in HOCM.

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78.). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8.). This can cause detrimental symptoms, impair functional status and reduce the quality of life.

Hypertrophic cardiomyopathy is another common heart disorder, usually genetic in origin, that may affect up to 600,000 people in the United States. The disorder, which is characterized by left ventricular hypertrophy, is usually not progressive, but a small subset of patients develop serious complications, such as progressive heart failure, atrial fibrillation, and sudden cardiac death.

Hyperpolarization-activated cyclic nucleotide-gated (HCN) channels are voltage-gated channels encoded by the HCN1-4 gene family. These channels are primarily expressed in the heart and in the central and peripheral nervous systems. HCN channels conduct K+ and Na+ ions at a ratio of 3:1 to 5:1. They are activated by hyperpolarization of membrane voltage to −50 mV or below, and conduct the hyperpolarization-activated current, termed I_{f} in heart. In the sino-atrial node (SAN), I_{f} plays an essential role in setting the heart rate and mediating its autonomic control.

HCN4 constitutes the predominant isoform in the sino-atrial node (SAN) at both transcript and protein level.

Gain of function variants in HCN4 have been shown to cause rhythm abnormalities, including symptomatic or asymptomatic bradycardia ventricular premature beats, tachycardia-bradycardia syndrome and atrial fibrillation (AF), complete atrioventricular (AV) block, long QT syndrome (LQTS) and torsades de pointes.

Drugs that specifically block HCN channels, e.g., ivabradine, slow the diastolic depolarisation of pacemaker cells, hence cardiac rate, with limited adverse cardiovascular side effects. Selective and quantitatively controlled slowing of heart rate provides an important therapeutic advantage in a variety of cardiac conditions.

### Targeting HCN3 for the Prevention and or Treatment of AFIB; Treatment, e.g., Rate Control, in HOCM.

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78.). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8.). This can cause detrimental symptoms, impair functional status and reduce the quality of life.

Hypertrophic cardiomyopathy is another common heart disorder, usually genetic in origin, that may affect up to 600,000 people in the United States. The disorder, which is characterized by left ventricular hypertrophy, is usually not progressive, but a small subset of patients develop serious complications, such as progressive heart failure, atrial fibrillation, and sudden cardiac death.

Hyperpolarization-activated cyclic nucleotide-gated (HCN) channels are voltage-gated channels encoded by the HCN1-4 gene family. These channels are primarily expressed in the heart and in the central and peripheral nervous systems. HCN channels conduct K+ and Na+ ions at a ratio of 3:1 to 5:1. They are activated by hyperpolarization of membrane voltage to −50 mV or below, and conduct the hyperpolarization-activated current, termed I_{f} in heart. In the sino-atrial node (SAN), I_{f} plays an essential role in setting the heart rate and mediating its autonomic control.

Although the cardiac expression of HCN3 channels is low, a ventricular phenotype caused by global deletion of HCN3 has been described. Epicardial myocytes of HCN3 knockouts displayed a reduction of I_{f} density by about 30% and a shortening of action potential duration caused by changes during the late repolarization phase. ECG recordings displayed a slight prolongation of the QT interval combined with increased T-wave amplitudes. These alterations were present only at low heart rates.

Thus, HCN3 contributes to the resting membrane potential and acts as a functional antagonist of hyperpolarizing K currents in late repolarization. Lack of this activity leads to a shortening of action potential duration.

### Targeting KCNA5 for the Prevention and or Treatment of AFIB, e.g., AFIB in Congestive Heart Failure (CHF).

Atrial fibrillation (AF) is the most common cardiac rhythm disorder in clinical practice. During the lifetime of men and women aged ≥40 years, there is about 25% risk for the development of AF. This arrhythmia may result in irregular ventricular response, tachycardia-mediated cardiomyopathy, heart failure and thromboembolism. AF accounts for nearly one-third of strokes in individuals above 65 years of age, and is also an independent predictor of mortality. AF is often associated with structural heart diseases or systemic disorders, such as hypertension, coronary artery disease, heart failure, rheumatic heart disease, hyperthyroidism and cardiomyopathies. However, in nearly 10-20% of cases, the underlying etiology for AF cannot be identified by routine examination, and such AF is termed ‘idiopathic’.

Heart failure (HF), including CHF and HF-pEF, affects an estimated 30-50 million patients worldwide. Despite recent therapeutic advances, its prevalence is increasing, partly due to a fall in mortality, but also from higher rates of major co-morbidities such as obesity, diabetes, and age. A major factor underlying cardiac dysfunction in HF resides in second messenger signaling defects coupled to 3′,5′-cyclic adenosine and guanosine monophosphate (cAMP, cGMP). Cyclic AMP stimulates protein kinase A (PKA) and exchange protein activated by cAMP (EPAC), acutely enhancing excitation-contraction coupling and sarcomere function. Cyclic GMP acts as a brake on this signaling by activating protein kinase G (PKG). Both cyclic-nucleotides have relevant vascular and fibroblast activity, reducing vessel tone, altering permeability and proliferation, and suppressing fibrosis. They are synthesized by adenylyl or guanylyl cyclases and degraded (hydrolyzed) by phosphodiesterases (PDEs), to provide tissue and cell specific intracellular nano-regulation.

K+ channels are members of a large family of transmembrane proteins that allow K+ to cross biological membranes selectively. Like Ca2+ and Na+ channels, voltage-gated K+ channels undergo conformational changes to open and close a gate in response to membrane depolarization. The K+ channel family is formed by a complex and diverse group of proteins that is known to exist in all three domains of organisms, eubacteria, archaebacteria, and eukaryotes. K+ channels have a wide range of functions that includes setting the resting membrane potential, modulating electrical excitability, and regulating cell volume.

Cardiac potassium channels maintain the rhythmicity of the heartbeat by repolarizing cardiomyocytes such that the electrical and contractile machineries stay in sync. They alternate between opened and closed conformations in response to the voltage difference across the membrane and form functional homotetrameric channels and heterotetrameric channels that contain variable proportions of KCNA1, KCNA2, KCNA4, KCNA5, and possibly other family members as well.

Dominant-negative mutations in KCNA5 have been demonstrated to fail to generate the ultrarapid delayed rectifier current vital for atrial repolarization and exerted an effect on wild-type current.

### KCNJ3 for the Prevention and or Treatment of AFIB.

As the population ages globally, atrial fibrillation (AF or AFIB) is predicted to affect 6-12 million people in the USA by 2050 and 17.9 million in Europe by 2060. Subjects with atrial fibrillation are 5 to 7 times more likely to have a stroke than the general population. Clots can also travel to other parts of the body (kidneys, heart, intestines), and cause other damage. Atrial fibrillation can also decrease the heart's pumping ability. The irregularity can make the heart work less efficiently. In addition, atrial fibrillation that occurs over a long period of time can significantly weaken the heart and lead to heart failure.

Sinus node cells, located in the right atrium, spontaneously produce an electric impulse (i.e., action potential) that propagates along the cardiac conduction system and causes contraction of the heart muscle. Thus, heart rate is precisely regulated within the proper range by both intrinsic and extrinsic mechanisms. The acetylcholine-activated potassium channel (I_{KACh} channel) expressed in the sinus node, atrium, and atrioventricular node contributes to heart rate slowing triggered by the parasympathetic nervous system. The IKACh channel is a heterotetramer of 2 inwardly rectifying potassium channel proteins, Kir3.1 and Kir3.4, encoded by the genes KCNJ3 and KCNJ5, respectively. As indicated above, KCNJ5 mutation has been associated with atrial fibrillation (AF). However, the molecular basis of IKACh channel pathology remains poorly understood and, to date, rare mutations showing a large effect have not been reported for cardiac diseases.

Autosomal dominant mutations in KCNJ3 have been associated with symptomatic sinus bradycardia, and chronic AF with slow ventricular response. Because I_{KACh} channels are expressed more abundantly in the atrium than in the ventricle, the blockage of IKACh is a target for atrial-selective AF therapy with a lower risk of ventricular arrhythmia.

### Targeting KCNJ4 for the Prevention and or Treatment of AFIB

As the population ages globally, atrial fibrillation (AF or AFIB) is predicted to affect 6-12 million people in the USA by 2050 and 17.9 million in Europe by 2060. Subjects with atrial fibrillation are 5 to 7 times more likely to have a stroke than the general population. Clots can also travel to other parts of the body (kidneys, heart, intestines), and cause other damage. Atrial fibrillation can also decrease the heart's pumping ability. The irregularity can make the heart work less efficiently. In addition, atrial fibrillation that occurs over a long period of time can significantly weaken the heart and lead to heart failure.

Potassium channels, such as KCNJ4, play an important role in regulating adult heart function through shaping the action potential and maintaining the rhythm of cardiac contraction.

KCNJ4 has been identified as a target for antiarrhythmic drugs, as it is expressed at ˜10-fold greater levels in the atria relative to the ventricles of animal model and expression of KCNJ4 Has been shown to be upregulated in left atrial appendage tissues from subjects having AFIB. In addition, gain-of-function mutations of KCNJ4 have been associated with familial forms of AF, and KCNJ4 upregulation contributes to the stabilization/perpetuation of AFIB.

### Targeting PLN for the Prevention and or Treatment of HF-rEF, Arrhythmia, and or Cardiomyopathy

Heart failure, such as heart failure with reduced ejection fraction (HF-rEF), is a major cause of death and disability. The hallmarks of heart failure are impaired cardiac contraction and relaxation accompanied by abnormalities in calcium handling and 0-adrenergic signaling (Lou, Q, et al. Adv Exp Med Biol. 2012; 740:1145-1174). In cardiomyocytes, cytosolic calcium regulates cardiac contraction and relaxation by an excitation-contraction coupling mechanism. The Ca2+ influx via L-type calcium channels elicits Ca2+-induced Ca2+ release from sarco(endo)plasmic reticulum (SR) through ryanodine receptors, and increased cytosolic Ca2+ leads to cardiac contraction. The sequestration of Ca2+ from the cytosol into the SR, which determines active relaxation, is caused by a calcium pump on the SR called SR Ca2+ ATPase (SERCA2a), the activity of which is regulated by a small phosphoprotein, phospholamban (PLN) (Kranias, E G, et al., Circ Res. 2012; 110(12):1646-1660).

In physiological conditions, β-adrenergic receptor (PAR) stimulation enhances myocyte contraction by activating cyclic adenosine monophosphate-dependent kinase (protein kinase A [PKA]), which phosphorylates multiple Ca2+ cycling proteins, including PLN. Phospholamban inhibits SERCA2a activity through protein-protein interaction. Phosphorylation of PLN by PKA alters its interaction with SERCA2a to activate Ca2+ reuptake to the SR, resulting in enhanced SR Ca2+ loading and Ca2+ cycling. In the failing myocyte, dysfunctional PAR signaling leads to less PKA activation and activation of alternate pathways, such as calcium/calmodulin-dependent kinase II signaling to cause pathological hypertrophy. Consequently, the usefulness of positive inotropic agents in HF is strongly limited, and direct activation of Ca2+ cycling, which can circumvent dysfunctional PAR activity, is required. Thus, inhibition of PLN is one of the most promising strategies in this context. Several reports have demonstrated that PLN inhibition alleviates cardiac failure in various animal models of cardiac pathologies, including myocardial infarction in rats, genetic cardiomyopathy in hamsters, and dilated cardiomyopathy in mice (Iwanaga, Y, et al., J Clin Invest. 2004; 113(5):727-736; Hoshijima, M, et al., Nat Med. 2002; 8(8):864-871; Minamisawa, S. et al., Cell. 1999; 99(3):313-322). In addition, modulation of PLN improves contractility in human cardiomyocytes from patients with advanced HF (del Monte, F, et al. Circulation. 2002; 105(8):904-907), suggesting that targeting PLN is a bona fide therapy for failing hearts.

In particular, the ablation of PLN in mice prevents SERCA2a inhibition and enhances cardiac contractility by increasing the SR Ca2+ store. The ablation of PLN also reverses heart failure in some cardiomyopathic animal models, indicating the possibility of therapeutic approaches. The overexpression of PLN in mouse heart depresses cardiac function and proves that only ˜40% of SERCA pumps are normally regulated by PLN in mouse heart. The superinhibition of SERCA by specific PLN mutants impairs cardiac function and leads to cardiac remodelling and early death if the effects of the mutation cannot be reversed by β-agonists. In human and animal models of heart failure, the PLN-SERCA inhibited complex increases. Interventions that diminish the PLN-SERCA complex have been beneficial in some mouse models of heart failure (MacLennan and Kranias. 2003. Nat Rev Mol Cell Biol 4:566-77).

Dilated cardiomyopathy (DCM) is the second most common cause of heart failure with reduced ejection fraction (HFrEF) after coronary artery disease. It has been estimated that up to 40% of DCM cases have a genetic cause. The p.(Arg14del) pathogenic variant of the PLN gene (PLN-R¹⁴del) is a Dutch founder mutation with a high prevalence in DCM and arrhythmogenic cardiomyopathy (ACM) patients. Cardiomyopathy caused by the p.(Arg14del) pathogenic variant of the PLN gene is characterized by intracardiomyocyte PLN aggregation and can lead to severe DCM. Depletion of PLN attenuated heart failure in several cardiomyopathy models. Specifically, PLN knockdown was shown to reduce protein aggregation, normalize autophagy markers, improve cardiomyopathy and survival (Eijgenraam et al. 2022. Int J Mol Sci 23:2427. 4). PLN knockdown also reversed the heart failure phenotype in a genetic dilated cardiomyopathy mouse model, and prevented progression of left ventricular dilatation and improved left ventricular contractility in rats with myocardial infarction (Grote Beverborg et al. 2021. Nat Comm 12:5180). PLN ablation was also shown to reduce susceptibility to ventricular arrhythmias in mouse model of catecholaminergic polymorphic ventricular tachycardia (Mazzocchi et al. 2016. J Physiol 594: 3005-3030).

Thus, inhibition of PLN is an effective strategy in treating and/or preventing genetic cardiomyopathy, arrhythmia, as well as heart failure, in particular HF with reduced ejection fraction (HF-rEF).

### Targeting CAMK2D for the Prevention and or Treatment of Heart Failure and or AFIB

CAMK2D has been shown to associate with the development of cardiac disease, such as heart failure, and arrhythmias (Maier and Bers, 2002, J. Mol. Cell. Cardiol. 34, 919-939; Swaminathan et al., 2012, Circ. Res. 110, 1661-1677). Animal models have shown proof-of-concept studies that transgenic overexpression of CAMK2D is sufficient to induce structural and electrical remodeling in the heart, leading to compromised contractility and increased risk for sudden cardiac death (Zhang et al., 2002, J. Biol. Chem. 277, 1261-1267; Wagner et al., 2011, Circ. Res. 108, 555-565). Likewise, genetic and chemical inhibition of CAMK2D has been shown to confer protection from the development of dilated cardiomyopathy and sustained contractile performance, following both pressure overload and ischemic stress (Backs et al., 2009, J Clin. Invest. 116, 1853-1864.; Ling et al., 2009, J. Clin. Invest. 119, 1230-1240.). Human heart failure has also been associated with an increased expression/activity of CAMK2D (Hoch et al., 1999, Circ. Res. 84, 713-721). The central role for CAMK2D in development of disease stems from its regulation of proteins involved in critical cell functions such Ca2+ cycling. CAMK2D has been implicated in pathologic phosphorylation of a number of Ca2+ handling proteins including phospholamban, leading to activation of the sarcoplasmic reticulum (SR) ATP-driven Ca2+ pump SERCA2a (Mattiazzi and Kranias, 2014, Front. Pharmacol. 5, 5.); the ryanodine receptor SR Ca2+ release channel (RyR2) (Witcher et al., 1991, J. Biol. Chem. 266, 11144-11152), promoting increased channel open probability and SR Ca2+ leak; and the L-type Ca2+ channel Cav1.2 and associated β-subunits, potentiating current amplitude and slowing inactivation (Hudmon et al., 2005, J. Cell Biol. 171, 537-547). Collectively, these events not only promote activation of hypertrophic remodeling cascades but also heighten the risk for inappropriate membrane potential depolarizations (afterdepolarizations) that serve as arrhythmia triggers (Wu et al., 2002, Circulation 106, 1288-1293).

In addition to association with heart failure, CAMK2D has also been found to be a GWAS locus for atrial fibrillation (Roselli C et al. 2018. Nat Genet; 50:1225-1233; Ramirez J et al. 2020. Am J Hum Genet 106:764-78). Pathological activation of CAMK2D promotes arrhythmia and heart failure (Veitch C R et al. 2021 Front Pharmacol 12: 695401; Nassal D et al. 2020. Front Pharmacol 11:35). In humans, CAMK2D levels and activity are increased in atrial fibrillation and heart failure. In animal models, sustained CAMK2D activation induces adverse structural and electrical remodeling of the heart via phosphorylation of target proteins. Pharmacological and genetic inhibition were shown to prevent these changes. Transgenic expression in the atria of a CAMK2D inhibitory peptide was shown to prevent adverse atrial structure and electrical remodeling (Liu Z et al. 2019. Heart Rhythm 16:1080-1088). In addition, CAMK2D knockout protects against pathological cardiac hypertrophy in a mouse model of heart failure (Backs J et al. PNAS 2009; 106:7:2342-2347).

Thus, suppressing CAMK2D expression is an effective strategy in treating and/or preventing heart failure and/or atrial fibrillation.

### Targeting PDE1 for the Prevention and or Treatment of CHF and or HF-pEF

Heart failure (HF), including CHF and HF-pEF, affects an estimated 30-50 million patients worldwide. Despite recent therapeutic advances, its prevalence is increasing, partly due to a fall in mortality, but also from higher rates of major co-morbidities such as obesity, diabetes, and age. A major factor underlying cardiac dysfunction in HF resides in second messenger signaling defects coupled to 3′,5′-cyclic adenosine and guanosine monophosphate (cAMP, cGMP). Cyclic AMP stimulates protein kinase A (PKA) and exchange protein activated by cAMP (EPAC), acutely enhancing excitation-contraction coupling and sarcomere function. Cyclic GMP acts as a brake on this signaling by activating protein kinase G (PKG). Both cyclic-nucleotides have relevant vascular and fibroblast activity, reducing vessel tone, altering permeability and proliferation, and suppressing fibrosis. They are synthesized by adenylyl or guanylyl cyclases and degraded (hydrolyzed) by phosphodiesterases (PDEs), to provide tissue and cell specific intracellular nano-regulation.

PDE1 is constitutively and robustly expressed in the heart. It is activated by a Ca2+/calmodulin-binding domain and provides a substantial percent of in vitro cAMP and cGMP hydrolytic activity in mammals, including humans.

It has been shown that inhibition of PDE1 prevents phenylephrine-induced myocyte hypertrophy in neonatal and adult rat ventricular myocytes reduces angiotensin II or TGF-induced activation of rat cardiac fibroblasts, and attenuates isoproterenol-induced interstitial fibrosis in mice. Cellular senescence in vascular smooth muscle myocytes leads to elevated PDE1 expression, and PDE1 inhibition restores vasodilatory responses to sodium nitroprusside in aging mice. PDE1 expression in vascular smooth muscle cells in vitro increases with the transition from the contractile to the synthetic phenotype, and PDE1 inhibition attenuates proliferation and migration of vascular smooth muscle cells in culture. PDE1 expression is increased in mouse vascular injury models in vivo and in neointimal smooth muscle cells of human coronary arteries, and injury-induced neointimal formation is reduced by PDE1 inhibition in coronary arteries of mice. Knockout of the PDE1C gene has antihypertrophic, antifibrotic, and antiapoptotic actions in mouse hearts. These observations suggest that PDE1 is a therapeutic target for cardiovascular disease. Indeed, recently a selective small molecule PDE1 inhibitor, ITI-214, was demonstrated to improve cardiac output by increasing heart contractility and decreasing vascular resistance in a Phase I/II study of heart failure patients.

### Targeting Myostatin for the Prevention and or Treatment of Myostatin-Related Muscle Dystrophy

Myostatin, also known as growth differentiation factor 8 (GDF8), is a negative regulator of muscle mass and a member of the TGF-β superfamily of proteins. Myostatin is initially synthesized by myocytes as a pre-promyostatin molecule composed of an N-terminal signal sequence (for secretion), an N-prodomain region (essential for proper folding of myostatin and subsequently proteolytically processed), and the biologically active C-terminal domain. The precursor pre-promyostatin must undergo proteolytic cleavage to form the biologically active myostatin molecule, which exists as a disulfide-linked dimer of two C-terminal domains. The cleaved propeptide domain also plays a regulatory role through non-covalent binding to the active myostatin C-terminal domain to form an inactive latent myostatin complex. Myostatin is also capable of effecting a non-canonical signaling cascade involving the cellular energy-sensing enzyme AMP-activated kinase (AMPK) and a regulatory protein kinase transforming growth factor-β-activated kinase 1.

Genetic deletion of myostatin has been associated with increasing muscle mass in mice, cattle, dogs, horses, and other species, indicating its evolutionary conservation (McPherron A C, et al., Nature 1997; 387:8390). Discovery of a hypermuscular child who was homozygous for an splice site mutation, which resulted in a premature stop codon, suggested that inhibition of myostatin might confer therapeutic benefits for muscle wasting disease in humans (Schuelke M, et al. New Engl J Med 2004; 350:26822688).

Various myostatin inhibitors have been developed and evaluated as potential treatments for different types of muscular dystrophy. These inhibitors have been shown to ameliorate the phenotype of muscular dystrophy, e.g., by improving muscle mass and strength.

### Targeting CHRNA1, CHRNB1, CHRND, CHRNE and CHRNG for the Prevention and or Treatment of CMS

Congenital myasthenic syndromes (CMS) are a heterogeneous group of rare inherited neuromuscular disorders characterized by fatigable weakness of skeletal muscle owing to compromised function of the neuromuscular junction (NMJ). The phenotype is caused by failure of transmission across this synapse connecting the nerve with the muscle, whereby an incoming nerve stimulus does not consistently lead to muscle excitation and contraction. Neuromuscular transmission is mediated by the generation of an action potential causing the release of acetylcholine from the nerve terminal into the synaptic cleft, its binding to the acetylcholine receptor (AChR) with the opening of its ion channel and the enzymatic breakdown of acetylcholine by acetylcholinesterase (AChE). The AChR controls electrical signalling between nerve and muscle cells by opening and closing a gate, membrane-spanning pore to trigger muscle contraction. It has five subunits of four different types: two alpha and one each of beta, gamma (or epsilon), and delta subunits ((i.e., CHRNE, CHRNA1, CHRNB1, CHRND, and CHRNG). Mutations affecting subunits of the AChR pore cause CMS in humans.

Pathophysiological mechanisms acting on any part of this chain and resulting in a reduction in the amount of acetylcholine released, the impairment of the AChR, reduction in the number of receptors or defective breakdown of acetylcholine may lead to CMS. The majority of CMS types are caused by defects in the AChR itself, but they can also result from causative variants affecting presynaptic proteins or proteins associated with the synaptic basal lamina or variants causing defects in endplate development and maintenance or defects in protein glycosylation. Defective neuromuscular transmission presents clinically as fatigable weakness due to increasing impairment of transmission across the NMJ with repeated activation.

Generalized and fatigable skeletal muscle weakness is the most common clinical sign of CMS, but locus and allelic heterogeneity determine variable severity and additional symptoms. CMS can result from recessive missense, non-sense, or splice site and promoter region mutations in any of the AChR subunits, but most occur in the gamma (or epsilon) subunit.

Diagnosis of CMS is established with clinical and electrodiagnostic features and identification of a causative mutation. In some instances, a clinical diagnosis can be made without finding a causative gene (e.g., individuals who exhibit fatigable weakness, especially of ocular and other cranial muscles, at birth or early childhood). Clinical diagnosis may rely on history, clinical exams, blood tests, incremental or decremental responses or abnormal single-fiber EMG (SF-EMG) study results, lung function tests, polysomnography, the Tensilon test, and muscle biopsy. In rarer cases when symptoms manifest in adolescence or adulthood, symptom presentation may differ from that seen in infants and young children and can include proximal and axial muscle weakness associated with a decremental response requiring prolonged stimulation.

Mutations in about 32 genes that encode proteins involved in this signaling pathway are known to cause CMS. Eight proteins are associated with presynaptic CMS, four with synaptic CMS, fifteen with post-synaptic CMS, and five with glycosylation defects. Proteins affected in CMS have different functions, such as ion channels (AchR,), structural proteins (COL13A1, RAPSN), signalling molecules (LRP4, MUSK, DOK7), catalytic enzymes, sensor proteins, or transport proteins. Various gene mutations in presynaptic, synaptic, and postsynaptic proteins have been demonstrated in patients, with more than 50% of the mutations involving aberrations in postsynaptic AChR subunits (i.e., CHRNE, CHRNA1, CHRNB1, CHRND, and CHRNG). Mutations in RAPSN, COLQ, and DOK7 comprise another 35% to 50% of cases.

The CHRNA1 gene encodes the alpha-subunit of the nicotinergic, post-synaptic AchR. CHRNA1 mRNA undergoes alternative splicing and two splice variants (P3A− and P3A+) are produced. Mutations in CHRNA1 result in imbalance between the two splice variants with an increase in P3A+. CHRNA1 mutations reduce the number of AchR at the post-synaptic membrane. The pattern of inheritance is autosomal dominant if CHRNA1 mutations cause a slow channel CMS (SCCMS), or autosomal recessive in case of primary AchR-deficiency. The first CHRNA1-related CMS were reported in 2008. Patients presented already prenatally with growth retardation, reduced movements, edema, contractures, and postnatally with dysmorphism, muscle wasting, scoliosis, contractures, and pterygia. Antisense oligonucleotides (AONs) have been shown to restore the balance between the two splice variants and are thus expected to be beneficial in patients carrying such mutations.

The CHRNB1 gene encodes for the beta-subunit of the nicotinergic, post-synaptic acetylcholine receptor (AChR). Non-synonymous mutations in the human CHRNB1 gene encoding the cholinergic receptor nicotinic beta 1 subunit are known to cause dominant and recessive forms of CMS. The first mutations in CHRNB1 causing CMS were reported in a Brazilian study in 2008. The first patient published was a 28 year old male manifesting since birth with ptosis, ophthalmoparesis, dysphagia, proximal limb muscle weakness, scapular winging, weakness of axial muscles, wasting, and scoliosis. He showed a decremental response to RNS, had double discharges, and a myopathic EMG. The course was progressive but he benefitted from fluoxetine (Mihaylova V, et al. J Neurol Neurosurg Psychiatry. 2010; 81:973-977). The second patient carrying a CHRNB1 mutation was a 3wo male manifesting with ptosis, facial weakness, severe hypotonia, and respiratory insufficiency requiring assisted ventilation (Shen X M, et al., Hum Mutat. 2016; 37:1051-1059). The response to LF-RNS was decremental. In a Spanish study of a CMS cohort, a third patient with a CHRNB1 mutation was identified but no clinical details were provided (atera-de Benito D, et al., Neuromuscul Disord 2017. pii: S0960-8966(17)30475-3).

The CHRND gene encodes the delta-subunit of the nicotinergic, post-synaptic AchR. The first mutation in CHRND causing CMS was reported in a German patient with early-onset CMS manifesting with feeding difficulties, moderate, generalised weakness, and recurrent episodes of respiratory insufficiency provoked by infections (Müller J S, et al., Brain. 2006; 129:2784-2793). The second patient was a 20 year old female with moderate to severe myasthenic manifestations since birth (Shen X M, et al., J Clin Invest. 2008 May; 118(5):1867-76). She had a marked decremental response to LF-RNS. One of her siblings with a similar presentation had died at age 11 m. Two further patients were reported in a study of CMS patients from Israel but no clinical details were provided (Aharoni S, et al., Neuromuscul Disord. 2017 February; 27(2):136-140).

The CHRNE gene encodes for the epsilon-subunit of the AchR. The first mutation in the CHRNE gene causing a CMS has been reported already in 2000 (Sieb J P, et al., Hum Genet. 2000; 107:160-164). Since then various different types of mutations have been reported and it is estimated that up to half of the patients with a CMS carry a CHRNE mutation, thus representing the gene most frequently mutated in CMS. In a study of 64 CMS patients from Spain, CHRNE mutations were detected in 27% of the patients (Natera-de Benito D, et al., Neuromuscul Disord 2017. pii: S0960-8966(17)30475-3). In a study of 45 patients from 35 Israeli CMS families, CHRNE mutations were found in 7 kinships (Aharoni S, et al., Neuromuscul Disord. 2017 February; 27(2):136-140). In a study of 23 families with CMS from Maghreb countries, the founder mutation c.1293insG was found in 60% of these patients (Richard P, et al., Neurology. 2008 Dec. 9; 71(24):1967-72). Type and severity of clinical manifestations of CHRNE mutations may vary considerably between affected families. Some patients may present with only ptosis whereas others may present with severe generalised myasthenia. Most patients present at birth with mildly progressive bulbar, respiratory, or generalized limb weakness with ptosis or ophthalmoplegia. Single patients may die prematurely in infancy from respiratory failure. Some patients may have myasthenic symptoms since birth and achieve ambulation late or not at all. Single patients present with a fluctuating course. Single patients develop severe scoliosis. RNS may be decremental or may be normal. Single-fiber EMG (SF-EMG) may reveal an increased jitter. Some patients may show repetitive CMAPs. Most patients respond favourably to AchE inhibitors.

The CHRNG gene encodes for the fetal gamma-subunit of the AchR. Mutations in the CHRNG gene cause CMS with multiple ptyerygia (lethal multiple pterygia syndrome (LMPS) or the Escobar variant of multiple pterygia syndrome (EVMPS)) (Hoffmann K, et al., Am J Hum Genet. 2006; 79:303-312). In a study of seven families with Escobar syndrome (contractions, multiple pterygia, respiratory distress), mutations in the CHRNG gene were detected in 12 family members. The female to male ratio was 7:5. Some patients presented with decreased fetal movements, facial weakness, respiratory distress, arthrogryposis, short stature, kyphosis/scoliosis, dysmorphism, high-arched palate, cleft palate, arachnodactyly, or cryptorchism. None presented with myasthenic manifestations postnatally. CHRNG mutations may be also responsible for the allelic disease fetal akinesia deformation sequence (FADS). In a study of 46 CMS patients from Spain, five carried a mutation in the CHRNG gene (Natera-de Benito D, et al., Neuromuscul Disord 2017. pii: S0960-8966(17)30475-3). They all presented with arthrogryposis and delayed motor milestones, and some of them with poor sucking. Interestingly, none of them received drugs usually given for CMS. In a study of three Iranian CHRNG-related CMS patients, no drug treatment was applied. One of the patients presented with short neck, mild axillar pterygia, elbows and knees, joint contractures, clenched hands with thumbs held across palm and club feet (varus). The patient had rockerbottom feet, with almost no movement in ankles. Facial dysmorphism included hemangioma over forehead and nose, strabismus, flat nasal bridge, and downturned corners of mouth (Kariminejad A, et al., BMC Genet. 2016 May 31; 17(1):71).

Overall, CMS can result from missense, non-sense, or splice site and promoter region mutations in any of the AChR subunits, but most occur in the gamma (or epsilon) subunit. The high frequency of mutations in the epsilon subunit compared with other subunits has been attributed to phenotypic rescue by substitution of the fetal gamma subunit for the defective epsilon subunit (Ohno K, et al. Hum Mol Genet. 1997; 6:753-66). Individuals harboring null mutations in both alleles of CHRNA1, CHRNB1, or CHRND cannot survive because no substituting sub-units exist and hence these individuals probably die in utero (Engel A G, et al., Lancet Neurol. 2015; 14:420-34). Patients with heterozygous or homozygous low-expressor mutations in the non-epsilon subunit are severely affected have high mortality in infancy or early childhood. Thus, given the importance of AchR in CMS, manipulation of AchR subunits are expected to ameliorate the CMS phenotype in patients.

### Targeting COL13A1 for the Prevention and or Treatment of CMS

Mutations in gene encoding synaptic proteins can cause CMS. Collegan XIII is a non-fibrillar transmembrane collagen which has been long recognized for its critical role in synaptic maturation of the neuromuscular junction. The COL13A1 gene encodes the α-chain of collagen XIII with a single transmembrane domain. COL13A1 is localised to the NMJ, where it is responsible for clustering of the AchR during myotube differentiation. Unlike most of the collagens, COL13A1 is anchored to the plasma membrane by a hydrophobic transmembrane segment. The presence of a proprotease recognition site in the ectodomain allows the C-terminus to be proteolytically cleaved into a soluble form that is part of the basal lamina.

Mutations in this gene manifest clinically as CMS, which has been reported in three patients from two families (Logan C V, et al. Am J Hum Genet. 2015; 97:878-85). Two of these patients manifested with congenital respiratory insufficiency, bulbar weakness, or facial weakness. All three patients presented with feeding difficulties, ptosis, limb weakness, and dysmorphism. Two patients each presented with spinal stiffness or distal joint laxity, and one patient with ophthalmoparesis and cognitive impairment. Two showed a decremental response to RNS and two an increased jitter. Two required non-invasive positive pressure ventilation.

COL13A1 loss-of-function mutations were also identified in six additional CMS patients from three unrelated families (Dusl M. et al., Journal of Neurology, 2019; 255: 1107-1112). The phenotype of these cases was similar to the previously reported patients including respiratory distress and severe dysphagia at birth that often resolved or improved in the first days or weeks of life. All individuals had prominent eyelid ptosis with only minor ophthalmoparesis as well as generalized muscle weakness, predominantly affecting facial, bulbar, respiratory and axial muscles. Response to acetylcholinesterase inhibitor treatment was generally negative while salbutamol proved beneficial. These data further support the causality of COL13A1 variants for CMS and suggest that this type of CMS might be clinically homogenous and requires alternative pharmacological therapy.

### Targeting LRP4 for the Prevention and or Treatment of CMS

Some CMS are due to mutations in genes encoding post-synaptic proteins. Post-synaptic CMSs represent the vast majority of the CMS subtypes. Post-synaptic CMS are subdivided into primary AchR deficiency, kinetic abnormalities of the AChR, and defects within the AChR-clustering pathway. Mutations in LRP4 cause defects within the AChR-clustering pathway.

The LRP4 gene encodes for lipoprotein receptor-related protein 4, which functions as a receptor for agrin. Agrin, which is released from motor nerve terminals, binds to LRP4 in muscle, stimulating the formation of a complex between LRP4 and muscle-specific kinase (MUSK), a receptor tyrosine kinase that acts as a master regulator of synaptic differentiation. LRP4, once clustered in the postsynaptic membrane as a consequence of MUSK activation, also signals directly back to motor axons to stimulate presynaptic differentiation. Activated MUSK together with DOK7 stimulates rapsyn to concentrate and anchor AchR at the post-synaptic membrane and interacts with other proteins implicated in the assembly and maintenance of the NMJ. LRP4 is thus essential for pre- and post-synaptic specialisation of the NMJ.

The first mutation in the LRP4 gene causing CMS was reported in 2014. A newborn female presented with respiratory arrest and feeding difficulties, and required feeding and ventilator support until 6 m of age. Motor milestones were delayed and she developed easy fatigability with temporary wheelchair-dependency. At ages 9 and 14y she presented with ptosis, ophthalmoparesis, and limb weakness. RNS evoked a decremental response, which improved upon application of edrophonium. AchE inhibitors worsened the clinical manifestations. A second kinship harbouring LRP4 mutations was reported in 2015. The two sisters, aged 34 and 20y, presented with delayed motor milestones, slight chewing and swallowing difficulties, and later developed limb weakness. Albuterol was highly effective.

### Targeting MUSK for the Prevention and or Treatment of CMS

Mutations in MUSK cause defects within the AChR-clustering pathway. MUSK encodes for a protein that is involved in endplate maturation, maintenance of the endplate functions, proper functioning of rapsyn, and functioning of the AchR. MUSK forms a co-receptor for agrin with LRP4. Activation of MUSK by agrin and DOK7 results in the recruitment of several downstream kinases and phosphorylation of the AChR β-subunit, leading to the reorganization of the actin cytoskeleton and AChR clustering. The fundamental role of the MUSK-signaling pathway is supported by the fact that mice deficient in agrin, MUSK, rapsyn or Dok-7 lack postsynaptic differentiation and die at birth from respiratory failure.

CMS due to MUSK mutations manifests as respiratory insufficiency, neonatal ptosis, proximal limb muscle weakness, and weak bulbar, facial, or ocular muscles. A 30yo Chinese male with the LGMD-type of MUSK-related CMS developed mild atrophy of the leg muscles. LF-RNS was decremental. Pyridostigmin deteriorated the clinical manifestations. Another male infant manifested with congenital respiratory failure requiring mechanical ventilation, axial weakness with head drop, facial weakness, proximal limb weakness, and ophthalmoparesis. Salbutamol was effective but 3,4-DAP had only a mild effect, and AchE inhibitors worsened the phenotype. In a female with congenital hypotonia and respiratory distress requiring mechanical ventilation for 8 m, respiratory distress and nocturnal apnea with vocal cord paralysis recurred at age 8y. 3,4-DAP was effective. In two Turkish brothers MUSK mutations manifested as LGMD-type CMS. MUSK-related CMS may also manifest as congenital ptosis and later in life with fatigability. In another patient with MUSK-related CMS and congenital respiratory insufficiency, albuterol was moderately effective but AchE inhibitor, 3,4-DAP, and ephedrine were ineffective.

### Targeting RAPSN for the Prevention and or Treatment of CMS

Mutations in RAPSN cause defects within the AChR-clustering pathway. RAPSN encodes for rapsyn, a post-synaptic membrane protein that anchors the nicotinic AchR to the motor endplate and also binds to β-dystroglycan. Rapsyn is essential for clustering of the AchR at the post-synaptic membrane and required for the phosphorylation of CHRNB1. Mutant mice lacking rapsyn show absence of aggregation of AChRs and lack of accumulation of cytoskeletal proteins such as β-dystroglycan, and utrophin.

RAPSN mutations are a common cause of post-synaptic CMS. Humans with mutations in the RAPSN gene are affected with a postsynaptic form of CMS characterized by impairment of the morphologic development of the postsynaptic region. The severity of symptoms in this form of CMS is variable. The most common of the RAPSN mutation is N88G, and patients are either homozygous or heterozygous for the N88K mutation (Ohno K, et al., (2002) Am J Hum Genet 70(4):875-885).

Clinically, patients present with fluctuating ptosis, occasionally bulbar symptoms, neck muscle and mild proximal limb muscle weakness. Infections can precipitate exacerbation of clinical manifestations. In single patients prominent hyperlordosis can occur. Usually, the response to AchE inhibitor is favourable but can be improved by adding 3,4 DAP. Fluoxetine may worsen the decremental response in single patients. In some patients general anesthesia may exacerbate muscle weakness. The overall course is stable with intermittent worsenings.

### Targeting DOK7 for the Prevention and or Treatment of CMS

Mutations in DOK7 cause defects within the AChR-clustering pathway, and are responsible for about 10-20% of all cases of CMS. The DOK7 (downstream-of-kinase) gene encodes for the protein DOK7, which is involved in signaling downstream of receptor and non-receptor phosphotyrosine kinases. DOK7 is a cytoplasmic activator of muscle-specific receptor-tyrosine kinase (MuSK). Both DOK7 and MuSK are required for neuromuscular synaptogenesis. Mutations in DOK7 underlie a congenital myasthenic syndrome (CMS) associated with small and simplified neuromuscular synapses likely due to impaired DOK7/MuSK signaling. The overwhelming majority of patients with DOK7 CMS have at least one allele with a frameshift mutation that causes a truncation in the COOH-terminal region of DOK7 and affects MuSK activation.

Concerning the frequency of DOK7-related CMS, it was the second most frequent subtype in a Brasilian cohort. Clinical onset is characterised by gait disturbance due to muscle weakness after normal motor milestones. Proximal limb muscles are more strongly affected than distal limb muscles (LGMD-like pattern). Congenital DOK7-related CMS may manifest as stridor due to vocal cord paralysis, occasionally requiring intubation and artificial ventilation. Occasionally, patients present with ptosis but only rarely with ophthalmoparesis. Fatigability is often absent but prolonged periods of weakness may occur. Feeding difficulties may require nasogastral tube feeding or even PEG implantation. Muscle biopsy may show lipidosis and defective branching of terminal axons, which results in a unique terminal axon contacting en passant post-synaptic cups. AchE inhibitors are usually ineffective and may even worsen clinical manifestations. Ephedrine (initially 25 mg/d and increased to 75-100 mg/d) seems to be an effective alternative. Salbutamol may be effective in DOK7-related CMS as well. Single patients profit from albuterol, which can prevent progression of muscle weakness in LGMD-type DOK7-related CMS.

### Targeting SCN4A for the Prevention and or Treatment of CMS

Mutations in SCN4A cause defects within the AChR-clustering pathway. SCN4A encodes for a post-synaptic Nav1.4 voltage-gated sodium channel responsible for the initiation and propagation of the action potential in the muscle fibres that results in muscle contraction.

Several allelic disorders of skeletal muscle are caused by mutations of SCN4A. Missense mutations with gain-of-function changes (too much inward Na+ current) are found in hyperkalemic periodic paralysis (HyperPP), paramyotonia congenita, and several variants of sodium channel myotonia. Leaky channels resulting from mutations of arginine residues in the voltage sensor domain cause hypokalemic periodic paralysis (HypoPP) type 2. These traits are all dominantly inherited.

Loss-of-function (LOF) mutations of SCN4A are associated with recessively inherited phenotypes. A congenital myasthenic syndrome (CMS) has been associated with missense mutations of SCN4A that cause a LOF by markedly enhancing channel inactivation. More recently, congenital myopathy with neonatal hypotonia has been reported in patients with null mutations in SCN4A. A homozygous null is embryonic lethal, while compound heterozygous mutations (null allele plus an LOF allele) result in congenital myopathy with survival to adulthood. Remarkably, family members with a single SCN4A null allele are healthy.

Phenotypically, mutations in this gene manifest in infancy with global hypotonia, impaired sucking, dysphagia, delayed postural and motor development and later in life with episodic, fluctuating muscle weakness like in periodic paralysis, bilateral facial palsy, ptosis, and ophthalmoparesis. Episodes of periodic weakness could not be triggered by exercise, rest, potassium loading, or food, like in periodic paralysis. In older patients, SCN4A-related CMS may manifest exclusively as easy fatigability. In a 20yo normokalemic female, SCN4A-related CMS manifested as sudden attacks of respiratory and bulbar paralysis since birth, lasting 3-30 min and recurring one to three times per month, delayed motor development, easy fatigability, ptosis, ophthalmoparesis, and later as persisting facial, truncal, or limb weakness. Some patients present with dysmorphism, such as high-arched palate, adduction deformity of the knees or ankles, and increased lumbar lordosis. Some patients are mentally retarded with cerebral atrophy on MRI. RNS may be normal but higher stimulus frequency may trigger a decremental response. AchE inhibitors are only marginally effective. Acetazolamide together with potassium was ineffective.

### Targeting DUX4 for the Prevention and or Treatment of FSHD

Facioscapulohumeral muscular dystrophy (FSHD) is an autosomal dominant disorder primarily characterized by asymmetric, progressive muscle weakness beginning at the face, shoulders, and upper limbs, which spreads to the lower regions of the body with age. It is the third most common muscular dystrophy, with about 1:8,000-1:22,000 people affected worldwide. Age of onset is variable, ranging from birth to adulthood. Patients with the rare infantile form of FSHD, presenting symptoms before 5 y of age, follow a more severe and rapid course of the disease. At present, FSHD is incurable.

The majority of FSHD patients (˜95%, FSHD1) have a contraction of the D4Z4 repeat array in chromosome 4q35. Each D4Z4 repeat contains the first two exons of the double homeobox protein 4 (DUX4) gene, with its third (and final) exon located immediately downstream of the array. The D4Z4 array is normally hypermethylated in the course of development. Studies show that the contraction relaxes the chromatin and demethylates DNA in this region, resulting in aberrant DUX4 expression in skeletal muscle.

The aberrant expression of DUX4 in skeletal muscle is thought to cause FSHD. DUX4 encodes a transcription factor that activates pathways involved in muscle degeneration and apoptosis, events observed in patient muscles. DUX4 also inhibits myogenic differentiation and increases the sensitivity of muscle cells to oxidative stress. DUX4 is normally expressed during early embryonic development, and is then effectively silenced in all tissues except the testis and thymus. Its reactivation in skeletal muscle disrupts numerous signalling pathways that mostly converge on cell death. Thus, DUX4 serves as an attractive therapeutic target and inhibition of DUX4 expression represents be a potential therapy approach for FSHD.

### Targeting DMPK for the Prevention and or Treatment of Myotonic Dystrophy

Mutations in the DMPK gene cause a form of myotonic dystrophy known as myotonic dystrophy type 1. Myotonic dystrophy is characterized by progressive muscle wasting and weakness. The muscle weakness associated with type 1 particularly affects muscles farthest from the center of the body (distal muscles), such as those of the lower legs, hands, neck, and face. People with this disorder often have prolonged muscle contractions (myotonia) and are not able to relax certain muscles after use.

The type of mutation that causes myotonic dystrophy type 1 is known as a trinucleotide repeat expansion. This mutation increases the size of the repeated CTG segment in the DMPK gene. People with myotonic dystrophy type 1 have from 50 to 1,000 CTG repeats in most cells. The number of repeats may be even greater in certain types of cells, such as muscle cells.

The mutated DMPK gene produces an altered version of mRNA, which is a molecular blueprint of the gene that is normally used to guide the production of proteins. Previous studies have shown that the altered mRNA traps proteins to form clumps within the cell. The clumps interfere with the production of many other proteins. These changes prevent muscle cells and cells in other tissues from functioning properly, leading to muscle weakness and the other features of myotonic dystrophy type 1.

The size of the trinucleotide repeat expansion is associated with the severity of signs and symptoms. People with the classic features of myotonic dystrophy type 1, including muscle weakness and wasting beginning in adulthood, usually have between 100 and 1,000 CTG repeats in their cells. People born with the more severe, congenital form of myotonic dystrophy type 1 tend to have more than 1,000 CTG repeats in their cells. People with the mild form of the condition usually have between 50 and 150 CTG repeats in their cells.

### Targeting GYS1 for the Prevention and or Treatment of Glycogen Storage Disease

Glycogen storage disease type 0 (also known as GSD 0) is a condition caused by the body's inability to form a complex sugar called glycogen, which is a major source of stored energy in the body. GSD 0 has two types: in muscle GSD 0, glycogen formation in the muscles is impaired, and in liver GSD 0, glycogen formation in the liver is impaired.

The signs and symptoms of muscle GSD 0 typically begin in early childhood. Affected individuals often experience muscle pain and weakness or episodes of fainting (syncope) following moderate physical activity, such as walking up stairs. The loss of consciousness that occurs with fainting typically lasts up to several hours. Some individuals with muscle GSD 0 have a disruption of the heart's normal rhythm (arrhythmia) known as long QT syndrome. In all affected individuals, muscle GSD 0 impairs the heart's ability to effectively pump blood and increases the risk of cardiac arrest and sudden death, particularly after physical activity. Sudden death from cardiac arrest can occur in childhood or adolescence in people with muscle GSD 0.

The GYS1 gene provides instructions for making muscle glycogen synthase; this form of the enzyme is produced in most cells, but it is especially abundant in heart (cardiac) muscle and the muscles used for movement (skeletal muscles). During cardiac muscle contractions or rapid or sustained movement of skeletal muscle, glycogen stored in muscle cells is broken down to supply the cells with energy.

Mutations in the GYS1 gene lead to a lack of functional glycogen synthase, which prevents the production of glycogen from glucose. Mutations that cause GSD 0 result in a complete absence of glycogen in muscle cells. As a result, these cells do not have glycogen as a source of stored energy to draw upon following physical activity or fasting. People with muscle GSD 0 do not have any stored energy, which leads to muscle pain, weakness, or episodes of fainting following moderate physical activity. Since there is no glycogen in cardiac muscle, affected individuals are also at an increased risk of cardiac arrest and sudden death, particularly after physical activity.

### Targeting SMN1 for the Prevention and or Treatment of Spinal Muscular Atrophy.

Spinal muscular atrophy is a genetic disorder characterized by weakness and wasting (atrophy) in muscles used for movement (skeletal muscles). It is caused by a loss of specialized nerve cells, called motor neurons that control muscle movement. The weakness tends to be more severe in the muscles that are close to the center of the body (proximal) compared to muscles away from the body's center (distal). The muscle weakness usually worsens with age. There are many types of spinal muscular atrophy that are caused by changes in the same genes. The types differ in age of onset and severity of muscle weakness; however, there is overlap between the types. Other forms of spinal muscular atrophy and related motor neuron diseases, such as spinal muscular atrophy with progressive myoclonic epilepsy, spinal muscular atrophy with lower extremity predominance, X-linked infantile spinal muscular atrophy, and spinal muscular atrophy with respiratory distress type 1 are caused by mutations in other genes.

Mutations in the SMN1 gene cause all types of spinal muscular atrophy described above. The number of copies of the SMN2 gene modifies the severity of the condition and helps determine which type develops. The SMN1 and SMN2 genes both provide instructions for making a protein called the survival motor neuron (SMN) protein. Normally, most functional SMN protein is produced from the SMN1 gene, with a small amount produced from the SMN2 gene. Several different versions of the SMN protein are produced from the SMN2 gene, but only one version is functional; the other versions are smaller and quickly broken down. The SMN protein is one of a group of proteins called the SMN complex, which is important for the maintenance of motor neurons. Motor neurons transmit signals from the brain and spinal cord that tell skeletal muscles to tense (contract), which allows the body to move.

### Targeting GAA for the Prevention and or Treatment of Pompe Disease

Pompe disease is to an inherited disorder caused by the buildup of a complex sugar called glycogen in the body's cells. The accumulation of glycogen in certain organs and tissues, especially muscles, impairs their ability to function normally. There are three types of Pompe disease, which differ in severity and the age at which they appear. These types are known as classic infantile-onset, non-classic infantile-onset, and late-onset. The classic form of infantile-onset Pompe disease begins within a few months of birth. Infants with this disorder typically experience muscle weakness (myopathy), poor muscle tone (hypotonia), an enlarged liver (hepatomegaly), and heart defects. Affected infants may also fail to gain weight and grow at the expected rate (failure to thrive) and have breathing problems. If untreated, this form of Pompe disease leads to death from heart failure in the first year of life. The non-classic form of infantile-onset Pompe disease usually appears by age 1. It is characterized by delayed motor skills (such as rolling over and sitting) and progressive muscle weakness. The heart may be abnormally large (cardiomegaly), but affected individuals usually do not experience heart failure. The muscle weakness in this disorder leads to serious breathing problems, and most children with non-classic infantile-onset Pompe disease live only into early childhood. The late-onset type of Pompe disease may not become apparent until later in childhood, adolescence, or adulthood. Late-onset Pompe disease is usually milder than the infantile-onset forms of this disorder and is less likely to involve the heart. Most individuals with late-onset Pompe disease experience progressive muscle weakness, especially in the legs and the trunk, including the muscles that control breathing. As the disorder progresses, breathing problems can lead to respiratory failure.

Mutations in the GAA gene cause Pompe disease. The GAA gene provides instructions for producing an enzyme called acid alpha-glucosidase (also known as acid maltase). This enzyme is active in lysosomes, which are structures that serve as recycling centers within cells. The enzyme normally breaks down glycogen into a simpler sugar glucose, which is the main energy source for most cells. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.

### Targeting MUC5B for the Prevention and or Treatment of Idiopathic Pulmonary Fibrosis

The MUC5B promoter polymorphism is the strongest and the most replicated genetic risk factor for IPF, is protective and predictive in this disease, and is involved in disease pathogenesis through an increase in MUC5B expression in terminal bronchi and honeycombed cysts (see, e.g., Yang, et al. Ann Am Thorac Soc. 2015 November; 12(Suppl 2): S193-S199).

### Targeting TSLP for the Prevention and or Treatment of Asthma

Thymic stromal lymphopoietin (TSLP) is an epithelial cell-derived cytokine implicated in the initiation and persistence of inflammatory pathways in asthma. Released in response to a range of epithelial insults (eg, allergens, viruses, bacteria, pollutants, and smoke), TSLP initiates multiple downstream innate and adaptive immune responses involved in asthma inflammation. Inhibition of TSLP is postulated to represent a novel approach to treating the diverse phenotypes and endotypes of asthma. Tezepelumab, the TSLP inhibitor farthest along in clinical development, is a human monoclonal antibody (IgG2λ) that binds specifically to TSLP, preventing interactions with its heterodimeric receptor and has been demonstrated to provide clinically meaningful improvements in asthma.

### Targeting IL33 for the Prevention and or Treatment of Asthma and Chronic Rhinosinusitis

Interleukin (IL)-33 is a key cytokine involved in type 2 immunity and allergic airway diseases. Abundantly expressed in lung epithelial cells, IL-33 plays critical roles in both innate and adaptive immune responses in mucosal organs. In innate immunity, IL-33 and group 2 innate lymphoid cells (ILC2s) provide an essential axis for rapid immune responses and tissue homeostasis. In adaptive immunity, IL-33 interacts with dendritic cells, Th2 cells, follicular T cells, and regulatory T cells, where IL-33 influences the development of chronic airway inflammation and tissue remodeling. The clinical findings that both the IL-33 and ILC2 levels are elevated in patients with allergic airway diseases demonstrate that inhibition od IL-33 is useful for treatment of these diseases.

### Targeting ALOX15 for the Prevention and or Treatment of Nasal Polyps and Chronic Rhinosinusitis

Nasal polyps (NP) are lesions on the nasal and paranasal sinus mucosa and are a risk factor for chronic rhinosinusitis (CRS). We performed genome-wide association studies on NP and CRS in Iceland and the UK (using UK Biobank data) with 4,366 NP cases, 5,608 CRS cases, and >700,000 controls. We found 10 markers associated with NP and 2 with CRS. We also tested 210 markers reported to associate with eosinophil count, yielding 17 additional NP associations. Of the 27 NP signals, 7 associate with CRS and 13 with asthma. Most notably, a missense variant in ALOX15 that causes a p.Thr560Met alteration in arachidonate 15-lipoxygenase (15-LO) confers large genome-wide significant protection against NP (P=8.0×10-27, odds ratio=0.32; 95% confidence interval=0.26, 0.39) and CRS (P=1.1×10-8, odds ratio=0.64; 95% confidence interval=0.55, 0.75). p.Thr560Met, carried by around 1 in 20 Europeans, was previously shown to cause near total loss of 15-LO enzymatic activity. Our findings identify 15-LO as a potential target for therapeutic intervention in NP and CRS (see, e.g., Kristjansson R P, et al. Nat Genet. 2019 February; 51(2):267-276).

Unless otherwise defined, all technical and scientific terms used herein have the same meaning as commonly understood by one of ordinary skill in the art to which this invention belongs. Although methods and materials similar or equivalent to those described herein can be used in the practice or testing of the RNAi agents and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

This invention is further illustrated by the following examples which should not be construed as limiting. The entire contents of all references, patents and published patent applications cited throughout this application, as well as the informal Sequence Listing and Figures, are hereby incorporated herein by reference.

### EXAMPLES

### Example 1: Design and Synthesis of αvβ6 Small Molecule Targeting Ligands

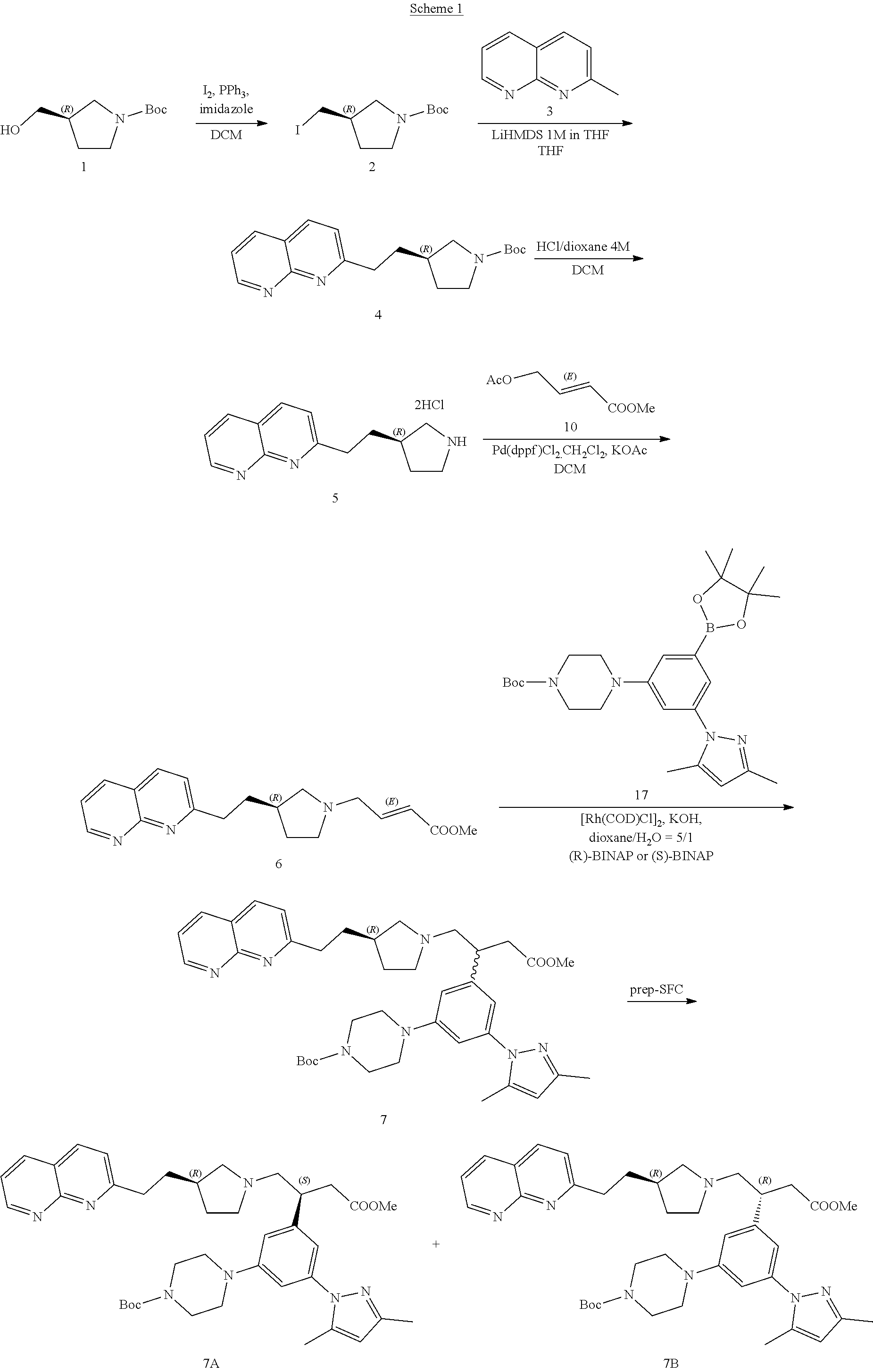

Compound 2: To a mixture of PPh₃ (237 g, 904 mmol, 1.30 eq) and imidazole (61.6 g, 904 mmol, 1.30 eq) in DCM (700 mL) was added I₂ (230 g, 904 mmol, 182 mL, 1.30 eq) portion-wise at 0-5° C. The reaction mixture was stirred at 0-5° C. for 0.5 hrs. To this reaction mixture was added to a solution of compound 1 (140 g, 695.61 mmol, 1.00 eq) in DCM (700 mL) dropwise below 15° C. and stirred the mixture overnight (12 hrs). TLC (Petroleum ether/Ethyl acetate=0/1, Rf=0.84) and Petroleum ether/Ethyl acetate=5/1 indicated no compound 1 was remained, and one major new spot with lower polarity was detected (Rf=0.43). The reaction mixture was filtered and concentrated under reduced pressure to give a residue. The residue was purified by column chromatography (SiO₂, Petroleum ether/Ethyl acetate=100/1 to 5/1). Compound 2 (203 g, 652 mmol, 93.8% yield) was obtained as a yellow liquid.
Compound 4: To a mixture of compound 2 (48.0 g, 154 mmol, 1.00 eq) and compound 3 (21.1 g, 147 mmol, 0.950 eq) in THF (740 mL) was added LiHMDS (1 M, 154 mL, 1.00 eq) drop-wise in one portion at 0° C. under N₂. The mixture was stirred at 0° C. for 3 hrs. TLC (Dichloromethane/Methanol=20/1, Rf=0.43) indicated some of compound 2 was remained, and one major new spot with larger polarity was detected. The reaction mixture was quenched by saturated solution of NH₄Cl 500 mL at 25° C., and then diluted with H₂O 200 mL and extracted with EtOAc 600 mL (200 mL×3). The combined organic layers were washed with brine 200 mL (100 mL×2), dried over Na₂SO₄, filtered and concentrated under reduced pressure to give a residue. The residue was purified by column chromatography (SiO₂, Petroleum ether/Ethyl acetate=100/1 to 3/1) and reversed-phase HPLC. Compound 4 (57.0 g, 174 mmol, 37.6% yield) was obtained as a brown oil with 3 batches.
Compound 5: To a solution of compound 4 (28.5 g, 87.0 mmol, 1.00 eq) in DCM (50 mL) was added HCl/dioxane (4 M, 109 mL, 5.00 eq) in one portion at 25° C. The mixture was stirred at 25° C. for 1 hr. TLC (Dichloromethane/Methanol=20/1, Rf=0) and LCMS indicated no compound 4 was remained, and one major new spot with larger polarity was detected. The reaction mixture was concentrated under reduced pressure to give a residue. The crude product was used into the next step without further purification. Compound 5 (53.0 g, crude, 2HCl) was obtained as a brown solid. ¹H NMR: MeOD, 400 MHz, 9.32 (dd, J=1.7, 5.1 Hz, 1H), 9.13 (dd, J=1.8, 8.3 Hz, 1H), 8.93 (d, J=8.6 Hz, 1H), 8.16-8.05 (m, 2H), 3.56 (dd, J=7.6, 11.6 Hz, 1H), 3.46 (ddd, J=4.0, 8.3, 11.9 Hz, 1H), 3.36 (br d, J=2.2 Hz, 1H), 3.32-3.23 (m, 2H), 3.03-2.94 (m, 1H), 2.56-2.42 (m, 1H), 2.41-2.28 (m, 1H), 2.25-2.02 (m, 2H), 1.79 (qd, J=9.0, 12.9 Hz, 1H). LCMS (ESI) calculated for C₁₄H₁₇N₃[M+H]⁺ m/z=228.14, found 228.0.
Compound 6: To a mixture of compound 5 (26.5 g, 88.3 mmol, 1.00 eq, 2HCl) and compound 10 (18.2 g, 115 mmol, 1.30 eq) in DCM (265 mL) was added KOAc (30.3 g, 310 mmol, 3.50 eq) and Pd(dppf)Cl₂·CH₂Cl₂ (7.21 g, 8.83 mmol, 0.100 eq) in one portion at 25° C. under N₂. The mixture was stirred at 25° C. for 2 hrs under N₂. TLC (Dichloromethane/Methanol=10/1, Rf=0.31) indicated no compound 5 was remained, and one major new spot with lower polarity was detected. LCMS indicated desired MS. The reaction mixture was filtered and concentrated under reduced pressure to give a residue. The residue was resolved in H₂O (100 mL). The mixture was acidified by 2N HCl until PH=4. The mixture was washed by EtOAc 300 mL (100 mL×3). The aqueous phase was basified by saturated solution of Na₂CO₃ until PH>8 and extracted with DCM 300 mL (100 mL×3), dried over Na₂SO₄, filtered and concentrated under reduced pressure to give a residue. The crude product was used into the next step without further purification. Compound 6 (52.0 g, 160 mmol, 90.5% yield) was obtained as a brown solid. ¹H NMR: CDCl₃, 400 MHz, 9.08 (dd, J=2.0, 4.3 Hz, 1H), 8.16 (dd, J=2.0, 8.1 Hz, 1H), 8.10 (d, J=8.3 Hz, 1H), 7.45 (dd, J=4.2, 8.0 Hz, 1H), 7.39 (d, J=8.3 Hz, 1H), 6.99 (td, J=6.1, 15.7 Hz, 1H), 5.99 (td, J=1.5, 15.7 Hz, 1H), 3.74 (s, 3H), 3.31-3.17 (m, 2H), 3.12-3.00 (m, 2H), 2.92-2.85 (m, 1H), 2.75-2.66 (m, 1H), 2.48 (dt, J=6.1, 8.7 Hz, 1H), 2.33-2.16 (m, 2H), 2.13-1.92 (m, 4H), 1.60-1.42 (m, 1H). LCMS (ESI) calculated for C₁₉H₂₃N₃O₂ [M+H]⁺ m/z=325.18, found 326.3.
Compound 7A: To a mixture of compound 6 (20.0 g, 61.5 mmol, 1.00 eq), KOH (3.62 g, 64.5 mmol, 1.05 eq), (R)-BINAP (3.83 g, 6.15 mmol, 0.100 eq) and compound 17 (34.1 g, 70.7 mmol, 1.15 eq) in dioxane (200 mL) and H₂O (20.0 mL) was added chlororhodium; (1Z,5Z)-cycloocta-1,5-diene (1.52 g, 3.07 mmol, 0.050 eq) in one portion at 25° C. under N₂. The mixture was heated to 100° C. and stirred for 4 hrs under N₂. TLC (Dichloromethane/Methanol=10/1, Rf=0.37) indicated no compound 6 was remained, and one major new spot with lower polarity was detected. LCMS indicated desired MS. The reaction mixture was concentrated under reduced pressure to remove dioxane. The residue was diluted with H₂O 200 mL and extracted with DCM 450 mL (150 mL×3). The combined organic layers were washed with brine 200 mL (100 mL×2), dried over Na₂SO₄, filtered and concentrated under reduced pressure to give a residue. The residue was purified by column chromatography (SiO₂, DCM/MeOH=100/1 to 5/1) and reversed-phase HPLC. The product was further separated by SFC (condition: column: DAICEL CHIRALPAK AD (250 mm×50 mm, 10 um); mobile phase: [0.1% NH₃H₂O ETOH]; B %: 40%-40%, 5.6 min). Compound 7A (14.5 g, 21.3 mmol, 34.6% yield) was obtained as a yellow solid. Chiral SFC indicated its structure. LCMS (ESI) calculated for C₃₉H₅₁N₇O₄[M+H]⁺ m/z=681.4, found 682.2.
Compound 7B: To a mixture of compound 6 (23.0 g, 70.7 mmol, 1.00 eq), KOH (4.16 g, 74.2 mmol, 1.05 eq), (S)-BINAP (4.40 g, 7.07 mmol, 0.100 eq) and compound 17 (39.2 g, 81.3 mmol, 1.15 eq) in dioxane (200 mL) and H₂O (20.0 mL) was added chlororhodium; (1Z,5Z)-cycloocta-1,5-diene (1.74 g, 3.53 mmol, 0.050 eq) in one portion at 25° C. under N₂. The mixture was heated to 100° C. and stirred for 4 hrs under N₂. TLC (Dichloromethane/Methanol=10/1, Rf=0.43) indicated no compound 6 was remained, and one major new spot with lower polarity was detected. The reaction mixture was concentrated under reduced pressure to remove dioxane. The residue was diluted with H₂O 200 mL and extracted with DCM 450 mL (150 mL×3). The combined organic layers were washed with brine 200 mL (100 mL×2), dried over Na₂SO₄, filtered and concentrated under reduced pressure to give a residue. The residue was purified by column chromatography (SiO₂, DCM/MeOH=100/1 to 5/1) and revers phase HPLC. The product was further separated by SFC (condition: column: DAICEL CHIRALPAK AD (250 mm×50 mm, 10 um); mobile phase: [0.1% NH₃H₂O ETOH]B %: 40%-40% 5.2 min). Compound 7B (15.0 g, 22.0 mmol, 31.1% yield) was obtained as a yellow solid. Chiral SFC indicated its structure.

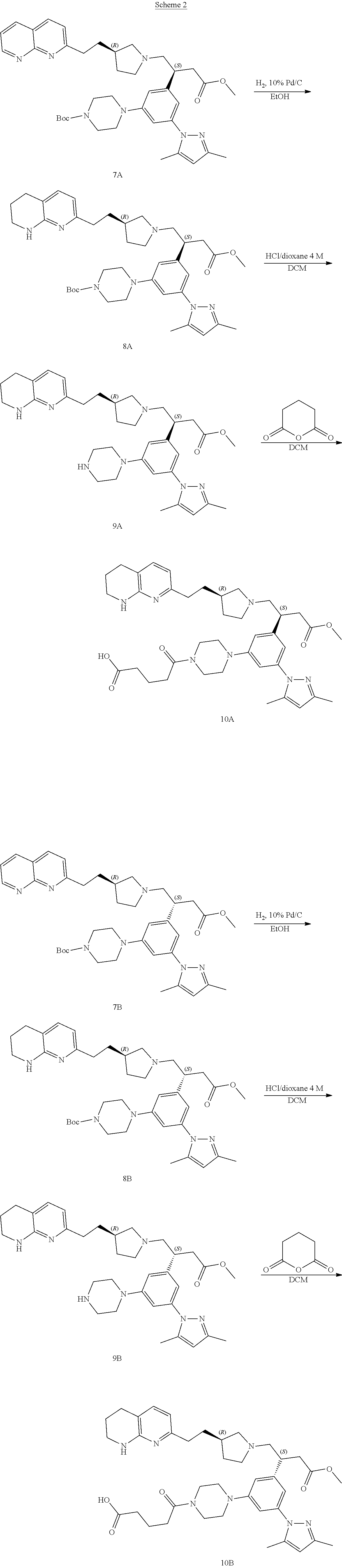

Compound 8A: To a solution of compound 7A (14.5 g, 21.3 mmol, 1.00 eq) in MeOH (150 mL) was added Pd/C (3.00 g, 21.3 mmol, 10% purity, 1.00 eq) in one portion at 25° C. The mixture was stirred at 25° C. for 12 hrs under H₂ (30 psi). TLC (Dichloromethane/Methanol=10/1, Rf=0.27) indicated no compound 7A was remained, and one major new spot with larger polarity was detected. LCMS indicated desired MS. The reaction mixture was filtered and concentrated under reduced pressure to give a residue. The residue was purified by prep-HPLC. Compound 8A (10.5 g, 15.3 mmol, 72.0% yield, 100% purity) was obtained as a yellow solid. ¹H NMR. MeOD, 400 MHz, 7.11 (d, J=7.3 Hz, 1H), 6.94 (s, 1H), 6.86 (s, 1H), 6.77 (s, 1H), 6.35 (d, J=7.3 Hz, 1H), 6.06 (s, 1H), 3.57 (s, 7H), 3.41-3.34 (m, 3H), 3.25-3.17 (m, 4H), 2.93-2.77 (m, 3H), 2.75-2.66 (m, 3H), 2.65-2.56 (m, 2H), 2.54-2.38 (m, 3H), 2.26 (d, J=6.7 Hz, 6H), 2.21-2.13 (m, 1H), 2.13-2.03 (m, 1H), 2.02-1.92 (m, 1H), 1.91-1.83 (m, 2H), 1.67 (q, J=7.5 Hz, 2H), 1.50 (s, 9H), 1.45-1.36 (m, 1H). LCMS (ESI) calculated for C₃₉H₅₅N₇O₄ [M+H]⁺ m/z=685.43, found 686.3.
Compound 9A: To a solution of 8A (1.50 g, 2.19 mmol, 1.00 eq) in DCM (10.0 mL) was added HCl/dioxane (4 M, 2.73 mL, 5.00 eq) at 25° C. The reaction mixture was stirred at 25° C. for 2 hrs. TLC (Dichloromethane/Methanol=10/1, Rf=0.06) indicated no 8A was remained, and one major new spot with larger polarity was detected. The reaction mixture was concentrated under reduced pressure. The residue was resolved in water (50 mL). The aqueous phase was basified by saturated solution of Na₂CO₃ until PH>8 and extracted with DCM 150 mL (50 mL×3), dried over Na₂SO₄, filtered, and concentrated under reduced pressure to give a residue. The crude product was used into the next step without further purification. Compound 9A (1.30 g, crude) was obtained as a yellow solid.
Compound 10A: To a mixture of compound 9A (1.30 g, 2.22 mmol, 1.00 eq) and tetrahydropyran-2,6-dione (253 mg, 2.22 mmol, 1.00 eq) in DCM (10.0 mL) was stirred at 25° C. for 12 hrs. TLC (Dichloromethane/Methanol=10/1, Rf=0.01) and HPLC indicated no compound 9A was remained, and one major new spot with lower polarity was detected. The reaction mixture was concentrated under reduced pressure to give a residue. Compound 10A (1.50 g, 2.14 mmol, 96.6% yield, 100% purity) was obtained as a yellow solid. ¹H NMR: CDCl₃, 400 MHz, 10.45 (br s, 1H), 7.18 (d, J=7.3 Hz, 1H), 6.93 (s, 1H), 6.83 (s, 1H), 6.73 (s, 1H), 6.21 (d, J=7.2 Hz, 1H), 5.97 (s, 1H), 3.91-3.81 (m, 1H), 3.77 (br d, J=13.4 Hz, 1H), 3.73-3.63 (m, 3H), 3.60 (s, 3H), 3.50-3.41 (m, 2H), 3.37-3.29 (m, 1H), 3.28-3.19 (m, 2H), 3.18-3.09 (m, 1H), 2.85-2.52 (m, 11H), 2.39-2.22 (m, 9H), 2.15-2.02 (m, 1H), 2.01-1.83 (m, 5H), 1.68 (q, J=7.4 Hz, 2H), 1.51-1.19 (m, 2H), 1.17-0.63 (m, 1H). LCMS (ESI) calculated for C₃₉H₅₃N₇O₅ [M+H]⁺ m/z=699.41, found 700.3.
Compound 8B: To a solution of compound 7B (15.0 g, 22.0 mmol, 1.00 eq) in MeOH (160 mL) was added Pd/C (4.00 g, 23.5 mmol, 10% purity) in one portion at 25° C. under H₂ (15 psi). The mixture was stirred at 25° C. for 12 hrs. TLC (Dichloromethane/Methanol=10/1, Rf=0.24) and LCMS indicated no compound 7B was remained, and one major new spot with larger polarity was detected. The reaction mixture was filtered and concentrated under reduced pressure to give a residue. The residue was purified by prep-HPLC (neutral condition) and SFC (column: Phenomenex-Cellulose-2 (250 mm×50 mm, 10 um); mobile phase: [0.1% NH₃H₂O ETOH]; B %: 50%-50%, 10.2 min). 8B (7.00 g, 10.2 mmol, 46.4% yield, 98.8% purity) was obtained as a yellow solid. ¹H NMR: MeOD, 400 MHz, 7.11 (d, J=7.3 Hz, 1H), 6.94 (s, 1H), 6.86 (s, 1H), 6.78 (s, 1H), 6.33 (d, J=7.4 Hz, 1H), 6.06 (s, 1H), 3.57 (s, 6H), 3.40-3.34 (m, 3H), 3.25-3.16 (m, 4H), 2.89-2.44 (m, 12H), 2.26 (d, J=6.9 Hz, 6H), 2.14-2.04 (m, 2H), 2.01-1.84 (m, 3H), 1.66 (q, J=7.4 Hz, 2H), 1.50 (s, 9H), 1.45-1.36 (m, 1H). LCMS (ESI) calculated for C₃₉H₅₅N₇O₄ [M+H]⁺ m/z=685.43, found 686.3.
Compound 9B: To a solution of 8B (1.00 g, 1.46 mmol, 1.00 eq) in DCM (2.00 mL) was added HCl/dioxane (4 M, 1.82 mL, 5.00 eq) at 25° C. The reaction mixture was stirred at 25° C. for 2 hrs. TLC (Dichloromethane/Methanol=10/1, Rf=0.06) indicated no 8B was remained, and one major new spot with larger polarity was detected. The reaction mixture was concentrated under reduced pressure. The residue was resolved in H₂O 50.0 mL. The aqueous phase was basified by saturated solution of Na₂CO₃ until PH>8 and extracted with DCM 150 mL (50.0 mL×3), dried over Na₂SO₄, filtered and concentrated under reduced pressure to give a residue. The crude product was used into the next step without further purification. Compound 9B (0.860 g, crude) was obtained as a yellow solid.
Compound 10B: To a mixture of compound 9B (860 mg, 1.47 mmol, 1.00 eq) and tetrahydropyran-2, 6-dione (168 mg, 1.47 mmol, 1.00 eq) in DCM (10 mL) was stirred at 25° C. for 12 hrs. TLC (Dichloromethane/Methanol=10/1, Rf=0.01) and HPLC indicated no compound 9B was remained, and one major new spot with larger polarity was detected. The reaction mixture was concentrated under reduced pressure to give a residue. 10B (1.10 g, crude, 99.3% purity) was obtained as a yellow solid. ¹H NMR: CDCl₃, 400 MHz, 10.86-10.16 (m, 1H), 7.18 (d, J=7.3 Hz, 1H), 6.88 (s, 1H), 6.82 (s, 1H), 6.70 (s, 1H), 6.19 (d, J=7.3 Hz, 1H), 5.98 (s, 1H), 3.95-3.84 (m, 1H), 3.83-3.68 (m, 2H), 3.60 (s, 3H), 3.45 (br t, J=5.5 Hz, 2H), 3.41-3.32 (m, 2H), 3.29-3.20 (m, 2H), 3.18-3.07 (m, 1H), 2.79-2.58 (m, 8H), 2.57-2.45 (m, 4H), 2.36 (dt, J=3.3, 6.5 Hz, 3H), 2.28 (d, J=10.7 Hz, 6H), 2.13-2.02 (m, 1H), 2.00-1.85 (m, 5H), 1.71 (td, J=7.0, 13.9 Hz, 1H), 1.65-1.55 (m, 1H), 1.37 (br dd, J=6.6, 12.0 Hz, 1H), 1.30-1.24 (m, 1H), 0.97-0.69 (m, 1H). LCMS (ESI) calculated for C₃₉H₅₃N₇O₅ [M+H]⁺ m/z=699.41, found 700.3.

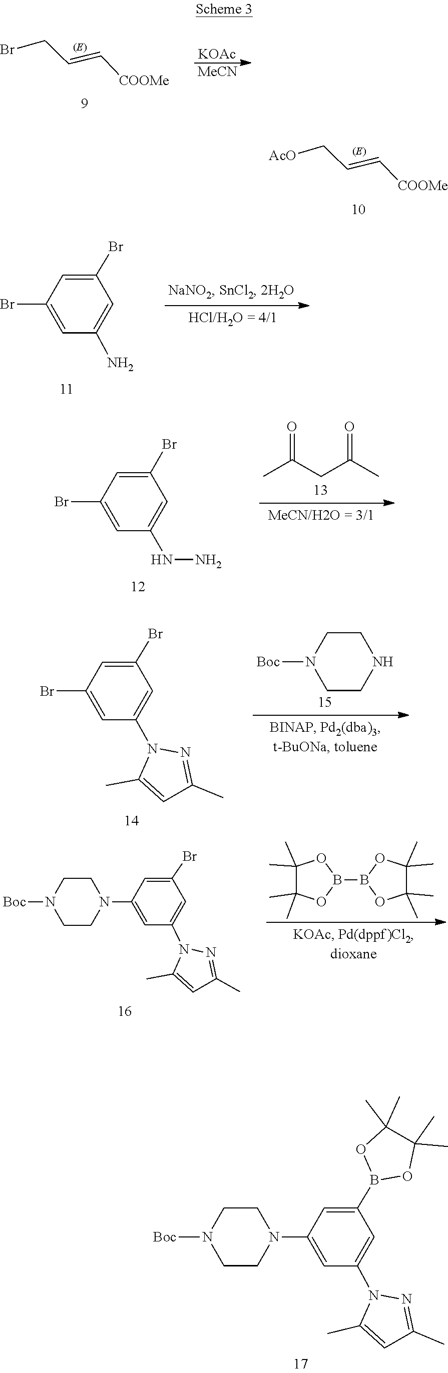

Compound 10: KOAc (40.3 g, 410 mmol, 1.05 eq) was added to a mixture of compound 9 (70.0 g, 391 mmol, 46.0 mL, 1.00 eq) in MeCN (350 mL) at 25° C. The mixture was stirred at 65° C. for 12 hrs. TLC (Petroleum ether/Ethyl acetate=5/1, Rf=0.45) showed that the reaction was complete. The mixture was concentrated to move the solvent. The residue was diluted with water (100 mL) and extracted with EtOAc (60.0 mL×3). The combine organic layer was dried over Na₂SO₄ and concentrated. Compound 10 (59.2 g, 374 mmol, 95.7% yield) was obtained as yellow oil. The product was used for the next step directly. ¹H NMR: CDCl₃, 400 MHz, 6.95 (td, J=4.6, 15.8 Hz, 1H), 6.04 (td, J=2.0, 15.8 Hz, 1H), 4.75 (dd, J=2.0, 4.6 Hz, 2H), 3.76 (s, 3H), 2.13 (s, 3H).
Compound 12: Compound 11 (250 g, 996 mmol, 1.00 eq) was dissolved in dilute HCl (4:1 in water, 1.00 L) and the mixture was cooled to 0° C. A cooled solution of NaNO₂ (75.6 g, 1.10 mol, 1.10 eq) in H₂O (400 mL) was added to the mixture at 0° C. and stirred for 30 minutes at that temperature. A mixture of SnCl₂·2H₂O (674 g, 2.99 mol, 3.00 eq) in HCl (500 mL) was added to the reaction at 0° C. and stirred at 0° C. for 1 hr. LCMS showed that the reaction was complete. The mixture was filtered and the filtrate cake was washed by water (200 mL). The filtrate cake was dried under vacuum. Compound 12 (353 g, crude, 2HCl) was obtained as yellow solid. LCMS (ESI) calculated for C₆H₆Br₂N₂[M+H]⁺ m/z=263.89, found 264.9, 266.8, 268.9.
Compound 14: Compound 13 (199 g, 1.99 mol, 204 mL, 2.00 eq) was added to a mixture of compound 12 (337 g, 994. mmol, 1.00 eq, 2HCl) in MeCN (1.50 L) and H₂O (500 mL) at 25° C. The mixture was stirred at 80° C. for 4 hrs. TLC (Petroleum ether/Ethyl acetate=10/1, Rf=0.73) and LCMS showed that the reaction was complete. The mixture was concentrated to remove MeCN. The residue was diluted with water (1.00 L) and basified to pH=7-8 by Na₂CO₃. It was extracted with EtOAc (1.00 L×3), organic layer was concentrated to give the crude product. The product was purified by MPLC (100-200 mesh silica gel, Petroleum ether/Ethyl acetate=100/1-10/1). Compound 14 (272 g, 824 mmol, 82.9% yield) was obtained as yellow solid. ¹H NMR: CDCl₃, 400 MHz, 7.64-7.62 (m, 1H), 7.59 (d, J=1.8 Hz, 2H), 6.01 (s, 1H), 2.35 (s, 3H), 2.29 (s, 3H). LCMS (ESI) calculated for C₁₁H₁₀Br₂N₂[M+H]⁺ m/z=327.92, found 328.9, 330.8, 332.9.
Compound 16: Pd₂(dba)₃ (6.65 g, 7.26 mmol, 0.02 eq) and BINAP (13.3 g, 21.4 mmol, 0.05 eq) was added to a mixture of compound 14 (133 g, 403.01 mmol, 1 eq), compound 15 (67.5 g, 362 mmol, 0.90 eq) and t-BuONa (77.5 g, 806 mmol, 2.00 eq) in anhydrous toluene (1.30 L) at 25° C. The mixture was stirred at 65° C. for 3 hrs under N₂. TLC (Petroleum ether/Ethyl acetate=5/1, Rf=0.28) showed that the reaction was complete. The mixture was washed by water (150 mL) and separated. The aqueous phase was extracted with EtOAc (100 mL×2). The combined organic layer was concentrated to give the crude product. The product was purified by MPLC (100-200 mesh silica gel, Petroleum ether/Ethyl acetate=100/1-5/1). Compound 16 (96.0 g, 220 mmol, 54.7% yield) was obtained as yellow solid.
Compound 17: Pd(dppf)Cl₂ (9.80 g, 13.4 mmol, 0.06 eq) was added to a mixture of compound 16 (98.0 g, 225 mmol, 1.00 eq), 4,4,5,5-tetramethyl-2-(4,4,5,5-tetramethyl-1,3,2-dioxaborolan-2-yl)-1,3,2-dioxaborolane (60.0 g, 236 mmol, 1.05 eq) and KOAc (44.2 g, 450 mmol, 2.00 eq) in dioxane (1.00 L) at 25-30° C. The mixture was stirred at 80° C. for 2 hrs under N₂. TLC (Petroleum ether/Ethyl acetate=3/1, Rf=0.26) showed that the reaction was complete. The mixture was washed by water (1.00 L) and separated. The aqueous phase was extracted with EtOAc (1.00 L×2). The combine organic layer was concentrated to give the crude product. The product was purified by MPLC (100-200 mesh silica gel, Petroleum ether/Ethyl acetate=4/1). Compound 17 (236 g, crude) was obtained as yellow solid. LCMS (ESI) calculated for C₂₆H₉₃BN₄O₄[M+H]⁺ m/z=482.3, found 483.1.

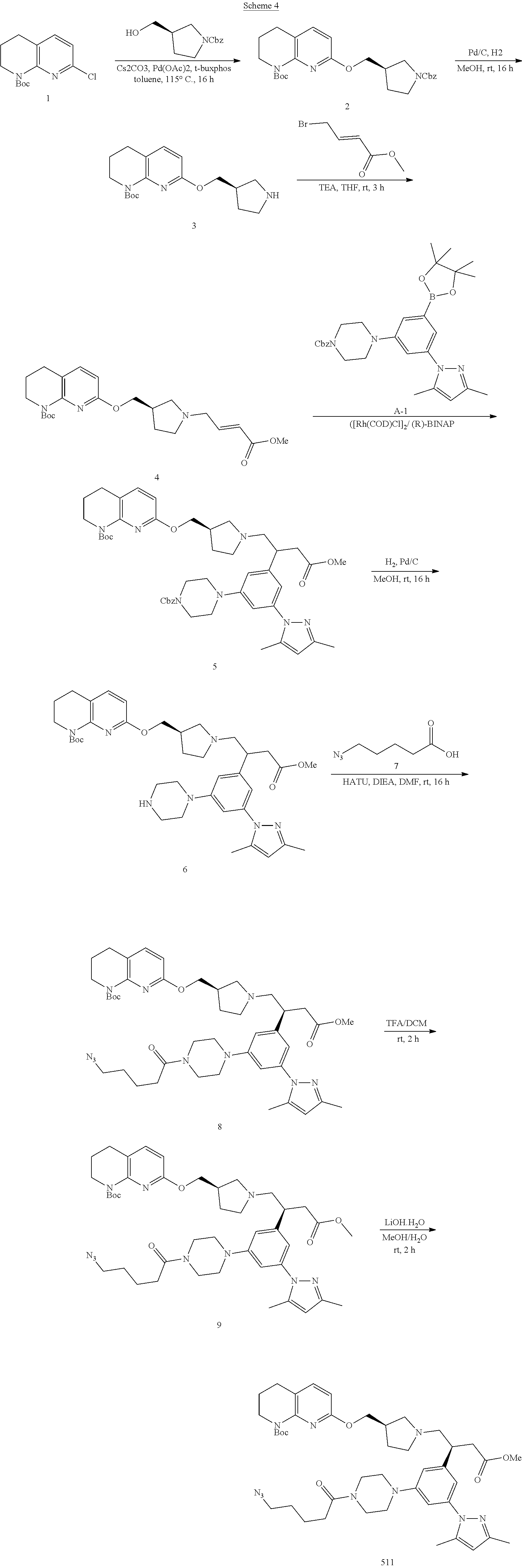

Compound 2: To a solution of 1 (9.1 g, 33.7 mmol, 1.0 eq) and 1-1 (9.5 g, 40.4 mmol, 1.2 eq) in toluene (120 ml) were added Cs₂CO₃ (22.0 g, 67.5 mmol, 2.0 eq), t-Bu-Xphots (717.5 mg, 1.69 mmol, 0.05 eq) and Pd(OAc)₂ (382 mg, 1.69 mmol, 0.05 eq) at 25° C. The solution was stirred at 115° C. for 16 h under N₂. After the reaction was completed, the solution was concentrated and the residue was purified by gel chromatography (SiO2, 100-200 mesh, DCM/MeOH=20:1) to give a yellow oil (9.0 g, 56.8% yield). MS(M+H⁺): 468.5
Compound 3: To a solution of 2 (9.0 g, 19.2 mmol, 1 eq) in MeOH (100 ml) was added Pd/C (10%, 1.0 g) at room temperature. The mixture solution was stirred at room temperature for 16 h under H₂. After the reaction was completed. Fileted and the filtrate was concentrated to give a yellow oil (6.0 g, 93.5% yield). MS(M+H⁺): 334.4
Compound 4: To a solution of 3 (5.8 g, 17.4 mmol, 1.0 eq) and 3-1 (3.18 g, 17.8 mmol, 1.02 eq) in THF (150 mL) was added TEA (3.5 g, 34.8 mmol, 2 eq) and stirred at room temperature for 16 hours under N₂. After the solution was completed, brine (70 ml) and water (70 ml) were added, extracted with EA (100 mL×2). The organic phase was dried over Na₂SO₄, filtered and concentrated to give a yellow oil (7.48 g, 99.6% yield). MS(M+H⁺): 432.2
Compound 5: To a solution of 4 (5.2 g, 12.0 mmol, 1 eq) and A-1 (7.43 g, 14.4 mmol, 1.2 eq) in dioxane (100 mL) was added [Rh(COD)CI]₂ (296 mg, 0.6 mmol, CAS:12092-47-6), (R)-BINAP (748 g, 1.2 mmol) and 3.6 M KOH (5.7 mL, 24 mmol), and stirred at 90° C. for 3 hours under N₂. The reaction mixture was diluted with water (100 mL), extracted with EA (100 mL×3). The organic layer was washed with brine (60 mL×2), dried over Na₂SO₄, concentrated under vacuum and purified by reverse chromatography (ACN in 0.1% TFA/water, 10% to 90%) to afford 5 (5.0 g, 50.4% yield, 6.08 mmol) as a yellow solid. MS(M+H⁺): 822.2
Compound 6: To a solution of 5 (3.5 g, 4.0 mmol) in MeOH (100 mL) was added Pd/C (350 mg) and stirred at room temperature for 16 hours under H₂. After the reaction was completed, remove Pd/C by filtered. The filtrate was concentrated under vacuum to give 6 (2.4 g, 3.49 mmol, 81.9% yield) as a yellow solid. MS(M+H⁺): 688.3
Compound 8: To a solution of 6 (2.4 g, 3.5 mmol), 5-azidopentanoic acid (550 mg, 3.85 mmol) and DIPEA (910 mg, 7 mmol) in DMF (15 mL) was added HATU (1.73 g, 4.55 mmol) and stirred at room temperature for 16 hours. The mixture was purified by reversed-phase chromatography (ACN in 0.1% TFA/water) to give 8 (2.6 g, 3.2 mmol, 91.4% yield) as a yellow solid. MS(M+H⁺): 813.2
Compound 9: To the solution of 8 (2.6 g, 3.2 mmol) in DCM (20 mL) was added TFA (10 ml) and stirred at room temperature for 2 hours. The reaction mixture was concentrated to obtain 9 (2.2 g, crude) as a yellow oil which will be used for the next step without purification. MS(M+H⁺): 713.2
Compound 511: To a solution of 9 (2.2 g, crude) in MeOH (20 mL) and water (20 mL) was added LiOH·H₂O (1.3 g, 31 mmol) and stirred at room temperature for 16 hours. The reaction mixture was concentrated under vacuum and purified by prep-HPLC (ACN in 0.1% FA/water) and SFC to give Target Compd 511 (520 mg, 0.74 mmol, 24.0%) as a white solid. 1H NMR (400 MHz, DMSO-d₆) δ 8.17 (s, 0.46H), 7.03 (d, J=8.0 Hz, 1H), 6.87 (s, 1H), 6.79 (s, 1H), 6.74 (s, 1H), 6.21 (s, 1H), 6.03 (s, 1H), 5.77 (d, J=8.0 Hz, 1H), 3.89-4.0 (m, 2H), 3.58 (br, 4H), 3.43 (br, 2H), 3.14-3.36 (m, 8H), 2.51-2.85 (m, 8H), 2.37-2.49 (m, 4H), 2.25 (s, 3H), 2.16 (s, 3H), 1.88 (m, 1H), 1.74 (m, 2H), 1.58 (m, 4H). 1.42 (m, 1H). MS(M+H⁺): 699.2

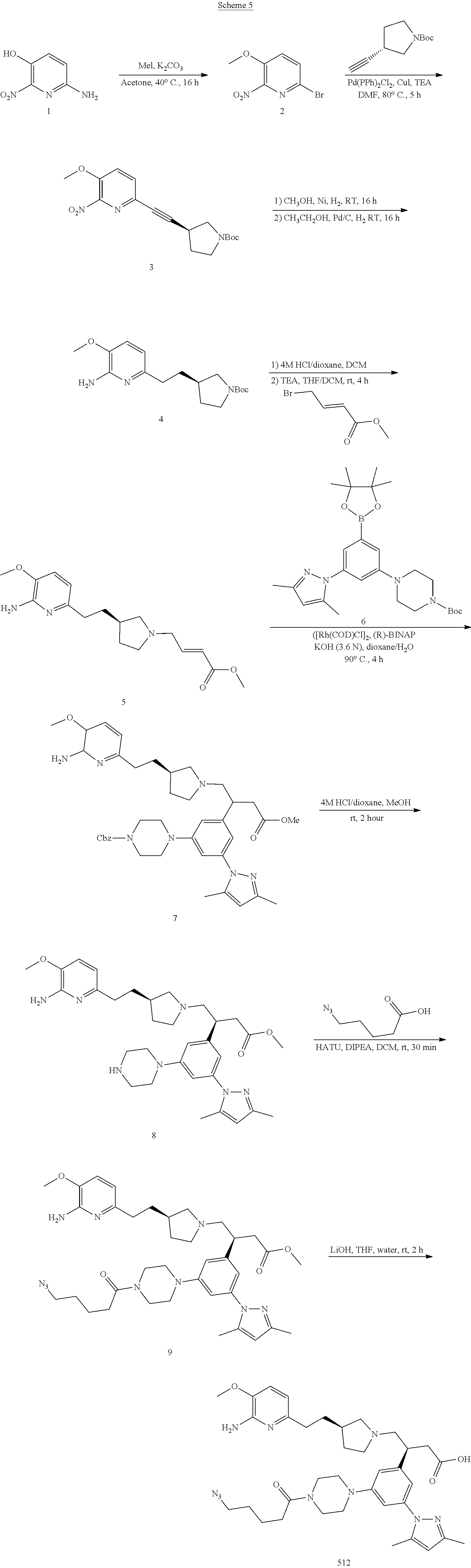

Compound 2: To the solution of 1 (15 g, 68.5 mmol) and CH₃I (11.7 g, 82.2 mmol) in DMF (100 mL) was added K₂CO₃ (11.5 g, 82.2 mmol), stirred at room temperature for 16 hours. The reaction mixture was diluted with EA (300 mL), washed with brine (100 mL×3), H₂O (100 mL×3) and LiCl (100 mL×3), dried over Na₂SO₄, concentrated under vacuum. to afford 2 (14.7 g, 90% yield, 63.09 mmol) as a yellow solid. MS(M+H⁺): 233
Compound 3: To a solution of 2 (10.2 g, 52.3 mmol), 6-bromo-3-methoxy-2-nitropyridine (11.1 g, 47.5 mmol) in DMF (200 mL) was added Pd(PPh₃)₂Cl₂ (3.3 g, 4.75 mmol), CuI (1.80 g, 9.50 mmol), TEA (14.40 g, 142.50 mmol), and stirred at 80° C. for 5 hours under N₂. The reaction mixture was cooled, diluted with water (300 mL), extracted with EA (100 mL×2). The organic layer was washed with LiCl (60 mL×2), dried over Na₂SO₄, concentrated under vacuum. The residue was purified by silica gel chromatography (EA in PE, 0% to 30%) to afford 3 (14 g, 84.95% yield, 40.35 mmol) as a brown solid. MS(M-Na): 370
Compound 4: To a solution of 3 (14 g, 40.35 mmol) in MeOH (500 mL) was added Raney Nickel (5 mL), and stirred at room temperature for 16 hours under H₂ (1 atm). The reaction mixture was filtered, the filtrate was concentrated under vacuum, the residue was added CH₃CH₂OH (1000 ml) was added Pd/C (1.4 g) and stirred at room temperature for three days under H₂ (1 atm) The reaction mixture was filtered, the filtrate was concentrated under vacuum to afford 4 (11.18 g, 86% yield, 34.83 mmol) as a brown solid. MS(M+H⁺): 322
Compound 5: To a solution of 4 (10.0 g, 31.15 mmol) in DCM (100 mL) was added 4N HCl/dioxane (100 mL), and stirred at room temperature for 1 hour. The reaction mixture was concentrated, the residue and TEA (9.46 g, 93.49 mmol) was dissolved with DCM (100 mL) and THF (100 mL), to this added methyl (E)-4-bromobut-2-enoate (5.68 g, 31.17 mmol), and stirred at room temperature for 4 hours. The residue was concentrated and diluted with EA (200 mL), washed with 1N HCl (50 mL×3), discard organic phase. The aqueous was adjust pH=8 with 10% NaOH at 0° C., extracted with DCM (100 mL×3). The organic layer was washed with brine (100 mL×2), dried over Na₂SO₄, concentrated under vacuum to afford 5 (6.0 g, 60.4%, 18.81 mmol) as a brown oil. MS(M+H⁺): 320.1
Compound 7: To a solution of 5 (6.0 g, 18.81 mmol) and 6 (10.9 g, 22.59 mmol) in dioxane (100 mL) was added 3.6 M NaOH (10.4 mL, 37.5 mmol), [Rh(COD)CI]₂ (0.46 g, 0.94 mmol, CAS: 12092-47-6), (R)-BINAP (1.17 g, 1.88 mmol), and stirred at 90° C. for 3 hours under N₂. The reaction mixture was diluted with water (100 mL), extracted with EA (100 mL×3). The organic layer was washed with brine (60 mL×2), dried over Na₂SO₄, concentrated under vacuum and purified by flash (MeOH in DCM, 0% to 20%) to afford 7 (3.2 g, 25.15% yield, 4.73 mmol) as a brown solid. MS(M+H⁺): 676.5
Compound 8: To a solution of 7 (3.2 g, 4.73 mmol) in MeOH (10 mL) was added 4N HCl/dioxane (30 mL), and stirred at room temperature for 1 hour. The reaction mixture was concentrated under vacuum to give 8 (crude, 4.73 mmol) as a yellow solid. MS(M+H⁺): 576.5
Compound 9: To a solution of 8 (crude, 4.73 mmol), 5-azidopentanoic acid (542 mg, 3.79 mmol) and DIPEA (1.83 g, 14.19 mmol) in DCM (50 mL) was added HATU (1.98 g, 5.21 mmol), and stirred at room temperature for 30 min. The reaction mixture was diluted with water (100 mL), extracted with DCM (100 mL×2). The organic layers were washed with brine (100 mL) dried over Na₂SO₄, concentrated to give 9 (4.56 g, crude) as a yellow solid. MS(M+H⁺): 701.5
Compound 512: To the solution of 9 (1.5 g, 2.14 mmol) in THF (20 mL) and water (10 mL) was added LiOH (450 mg, 10.71 mmol), and stirred at room temperature for 2 hours. The reaction mixture was adjusted pH=7-8 with 1N HCl, extracted with DCM (50 mL×3), concentrated and purified by prep-HPLC (ACN in 0.1% FA/water) to give Target Compd 512 (615 mg, 0.84 mmol, 39.3% yield) as alight yellow solid. 1H NMR (400 MHz, DMSO-d₆) δ 8.19 (s, 1H), 6.93-6.84 (m, 2H), 6.80 (s, 1H), 6.75 (s, 1H), 6.33 (dd, J=7.8, 3.4 Hz, 1H), 6.03 (s, 1H), 5.57 (d, J=13.5 Hz, 2H), 3.72 (s, 3H), 3.59 (s, 4H), 3.18 (d, J=21.0 Hz, 5H), 3.09-2.53 (m, 7H), 2.48-2.36 (m, 6H), 2.36-2.18 (m, 4H), 2.16 (s, 3H), 2.10-1.97 (m, 1H), 1.97-1.82 (m, 1H), 1.66-1.54 (m, 6H), 1.37 (dq, J=14.9, 7.5 Hz, 1H). MS(M+H⁺): 687.3

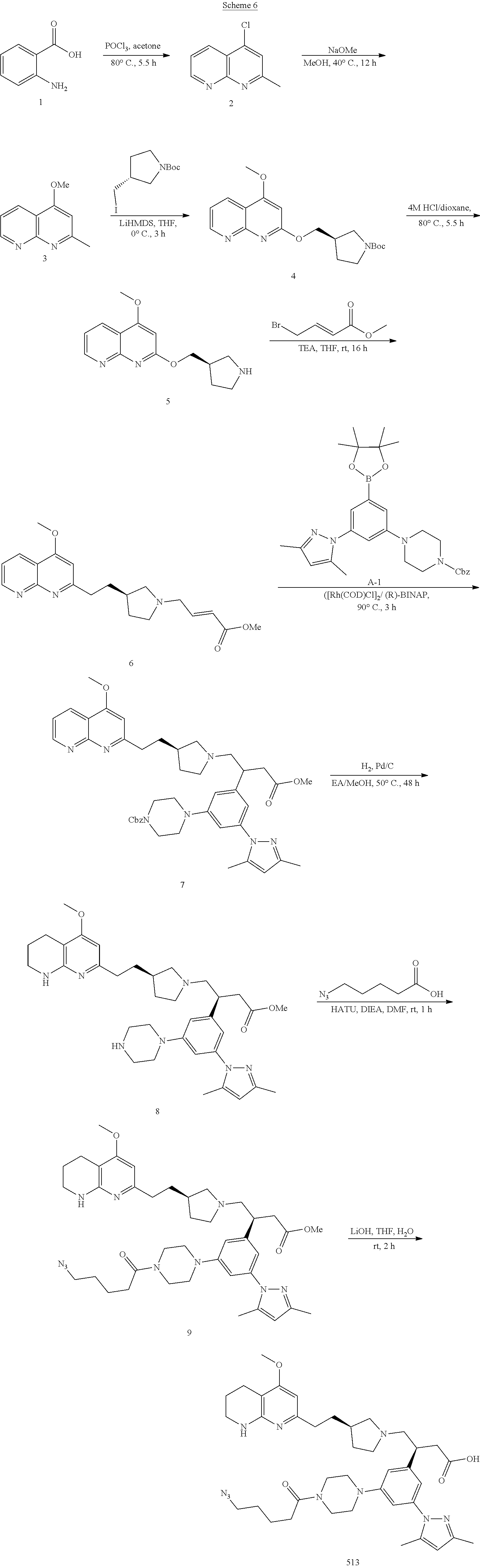

Compound 2: To a solution of 1 (27.5 g, 201.55 mmol) in acetone (46.28 g, 398.44 mmol) was added POCl₃ (100 mL). The reaction mixture was then heated for 5.5 h at 80 C. Next, the reaction mixture was carefully added to 200 mL ice water, which was then basified to pH 14 with NaOH (6 M aq) and stirred for 40 min (with additional base added to maintain pH 14). The aqueous material was split into two portions. Each portion was extracted with DCM (100 mL×6). The organic layers were combined and concentrated under vacuum to obtain 2 (crude, 201.55 mmol) as a brown solid. MS(M+H⁺): 179.1
Compound 3: To a solution of 2 (crude, 201.55 mmol) in MeOH (200 mL) was added NaOMe (24.8 mL, 403.1 mmol), and stirred at 40° C. for 16 hours. The reaction mixture was diluted with water (100 mL), extracted with EA (100 mL×3). The organic layer was washed with brine (60 mL×2), dried over Na₂SO₄, concentrated under vacuum and purified by reverse chromatography (DCM:MeOH 30:1) to afford 3 (3.3 g, 9.42% yield, 18.94 mmol) as a brown solid. MS(M+H⁺): 175.2
Compound 4: To a stirred solution of 3 (3.3 g, 18.94 mmol) and tert-butyl (R)-3-(iodomethyl) pyrrolidine-1-carboxylate (3.84 g, 12.63 mmol) in THF was cooled to 0° C. and treated under nitrogen with a solution of LiHMDS (18.9 ml, 18.9 mmol) in THF. The reaction mixture was stirred at 0° C. for 3 h. The reaction was quenched with saturated NH₄Cl solution (500 mL) and water (500 ml) and ethyl acetate (1 L) was added. The layers were separated and the aqueous phase was extracted with further ethyl acetate (1 L). The combined organic layers were dried (MgSO₄), filtered and evaporated in vacuum. The residual brown oil was purified by chromatography (PE: EtOAc 10:1) to give 4 (1.87 g, 27.62% yield, 2.23 mmol) as a brown solid. MS(M+H⁺): 358.2
Compound 5: To a solution of 4 (1.87 g, 5.23 mmol) in DCM (10 mL) was added 4M HCl/dioxane (10 mL), and stirred at room temperature for 2 hours. The reaction mixture was concentrated under vacuum to give 5 (crude, 6.88 mmol) as a brown oil. MS(M+H⁺): 258.2
Compound 6: To a solution of methyl (E)-4-bromobut-2-enoate (1.47 g, 8.24 mmol) in THF (10 mL) was added a solution of 5 (1.77 g, 6.87 mmol) and TEA (2.78 g, 24.48 mmol) in THF (15 mL), and stirred at room temperature for 16 hours. The reaction mixture was diluted with 1N HCl (300 mL), extracted with DCM (100 mL×2), discard organic phase. The aqueous was adjust pH=8 with 10% NaOH at 0° C., extracted with DCM (100 mL×3). The organic layer was washed with brine (100 mL×2), dried over Na₂SO₄, concentrated under vacuum to afford 6 (1.32 g, 54.10%, 3.72 mmol) as a brown oil. MS(M+H⁺): 356.2
Compound 7: To a solution of 6 (4.0 g, 11.25 mmol) and A-1 (7.6 g, 14.72 mmol) in dioxane (50 mL) was added [Rh(COD)CI]₂ (0.28 g, 0.56 mmol, CAS:12092-47-6), (R)-BINAP (0.70 g, 1.13 mmol) and 3.6 M NaOH (6.3 mL, 22.5 mmol), and stirred at 90° C. for 3 hours under N₂. The reaction mixture was diluted with water (100 mL), extracted with EA (100 mL×3). The organic layer was washed with brine (60 mL×2), dried over Na₂SO₄, concentrated under vacuum and purified by reverse chromatography (ACN in 0.1% TFA/water, 5% to 40%) to afford 7 (3.3 g, 39.4% yield, 4.43 mmol) as a brown solid. MS(M+H⁺): 746.2
Compound 8: To a solution of 7 (3.3 g, 4.43 mmol) in MeOH (30 mL) and EA (30 mL) was added Pd/C (600 mg). The mixture was stirred at room temperature for 48 hours under H₂ (1 atm). The reaction mixture was filtered and concentrated under vacuum to give 8 (2.3, 3.73 mmol, 84.2% yield) as a brown solid. MS(M+H⁺): 616.5
Compound 9: To a solution of 8 (2.3 g, 3.73 mmol), 5-azidopentanoic acid (530 mg, 3.70 mmol) and DIPEA (1.45 g, 11.24 mmol) in DCM (30 mL) was added HATU (1.85 g, 4.87 mmol). The mixture was stirred at room temperature for 1 hour. The mixture was purified by reversed-phase chromatography (5% to 50%, ACN in 0.1% TFA/water) to give 9 (2.0 g, 2.70 mmol, 72.4% yield) as a yellow solid. MS(M+H⁺): 741.5
Compound 513: To the solution of 9 (2.0 g, 2.7 mmol) in THF (20 mL) and water (5 mL) was added LiOH·H₂O (568 mg, 13.52 mmol), and stirred at room temperature for 2 hours. The reaction mixture was concentrated and purified by prep-HPLC (ACN in 0.1% FA/water) to give Target Compd 513 (590 mg, 0.81 mmol, 30% yield) as a white solid. ¹H NMR (400 MHz, DMSO-d₆) δ 6.86 (s, 1H), 6.79 (s, 1H), 6.74 (s, 1H), 6.19 (s, 1H), 6.10 (d, J=7.4 Hz, 1H), 6.03 (s, 1H), 3.73 (s, 3H), 3.58 (s, 5H), 3.27-3.12 (m, 8H), 2.94-2.86 (m, 1H), 2.79 (dd, J=16.0, 7.5 Hz, 2H), 2.70 (dd, J=16.0, 7.1 Hz, 1H), 2.63-2.52 (m, 2H), 2.48-2.35 (m, 8H), 2.26 (s, 3H), 2.16 (s, 3H), 2.05 (dt, J=14.6, 7.3 Hz, 1H), 1.96-1.84 (m, 1H), 1.70 (dd, J=11.1, 5.9 Hz, 2H), 1.67-1.53 (m, 6H), 1.36 (dd, J=11.7, 7.1 Hz, 1H). MS(M+H⁺): 727.3

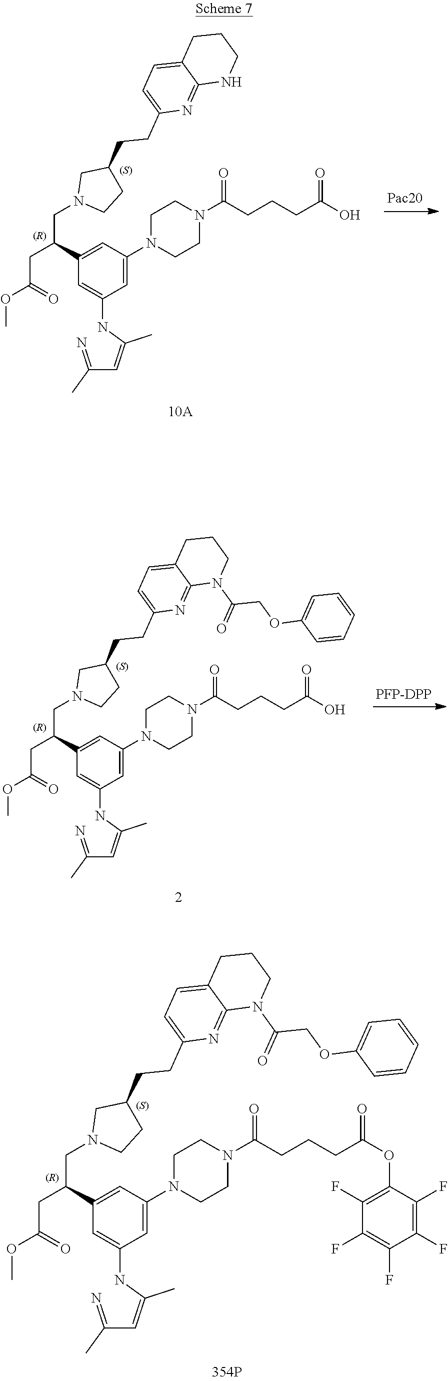

Compound 2: To a stirred solution of 10A (5 g, 7.14 mmol) in tetrahydrofuran (100 mL) in a round bottom flask at room temperature, phenoxyacetic anhydride (6.14 g, 21.43 mmol) was added. After the solution had stirred for 60 h the solvent was removed under vacuum leaving a residue with a mass of 11.63 g. This residue was taken up in DCM (100 mL), washed with sat. NH₄Cl (100 mL) and brine (100 mL), dried over Na₂SO₄, and the solvent was removed under vacuum affording 11.15 g of crude product 2 which was used in the next step without further purification. MS (ESI+APCI), calculated for C₄₇H₅₉N₇O₇ [M+H]⁺ exact mass m/z=834.45, found 834.5.
Compound 354P: To a stirred solution of crude product 2 (11.15 g) in DCM (75 mL), EDC (6.85 g, 35.72 mmol) was added. The mixture was stirred at rt for 10 min and then pentafluorophenol (6.57 g, 35.72 mmol) and 4-(dimethylamino)pyridine (261.84 mg, 2.14 mmol) were added. The reaction was stirred at room temperature for 18 h and then water (75 mL) was added. This mixture was transferred to a separatory funnel and the layers were separated. The organic layer was washed with brine (75 mL), dried over Na₂SO₄, and concentrated under vacuum to a mass of 16.83 g. The crude residue was taken up in DCM and purified on a 220 g silica gold column with a 5-55% THF/DCM gradient affording 4.33 g (61% yield) of pure product 354P as a white foam. ¹H NMR (600 MHz, CD₃CN) δ 7.44 (d, J=7.6 Hz, 1H), 7.24 (dd, J=8.8, 7.3 Hz, 2H), 6.90 (t, J=7.4 Hz, 2H), 6.81 (d, J=1.5 Hz, 1H), 6.81-6.77 (m, 3H), 6.72 (t, J=1.6 Hz, 1H), 5.99 (s, 1H), 5.25 (s, 2H), 3.83 (t, 2H), 3.66 (t, J=5.3 Hz, 2H), 3.58 (t, J=5.3 Hz, 2H), 3.51 (s, 3H), 3.28 (p, J=7.8 Hz, 1H), 3.19 (t, J=5.2 Hz, 2H), 3.14 (t, J=5.3 Hz, 2H), 2.85-2.77 (m, 4H), 2.77-2.73 (m, 3H), 2.71-2.60 (m, 4H), 2.54-2.45 (m, 4H), 2.42-2.26 (m, 1H), 2.24 (s, 3H), 2.23-2.21 (m, 1H), 2.19 (s, 3H), 2.11-2.03 (m, 1H), 1.99 (p, J=7.3 Hz, 2H), 1.94 (dt, J=4.9, 2.5 Hz, 1H), 1.89 (dt, J=12.2, 6.4 Hz, 3H), 1.77-1.67 (m, 2H), 1.43-1.35 (m, 1H). MS (ESI+APCI), calculated for C₅₃H₅₈F₅N₇O₇[M+H]⁺ exact mass m/z=1000.44, found 1000.2.
   Synthesis for Trivalent Scaffold of αvβ6 Integrin Targeting Ligand

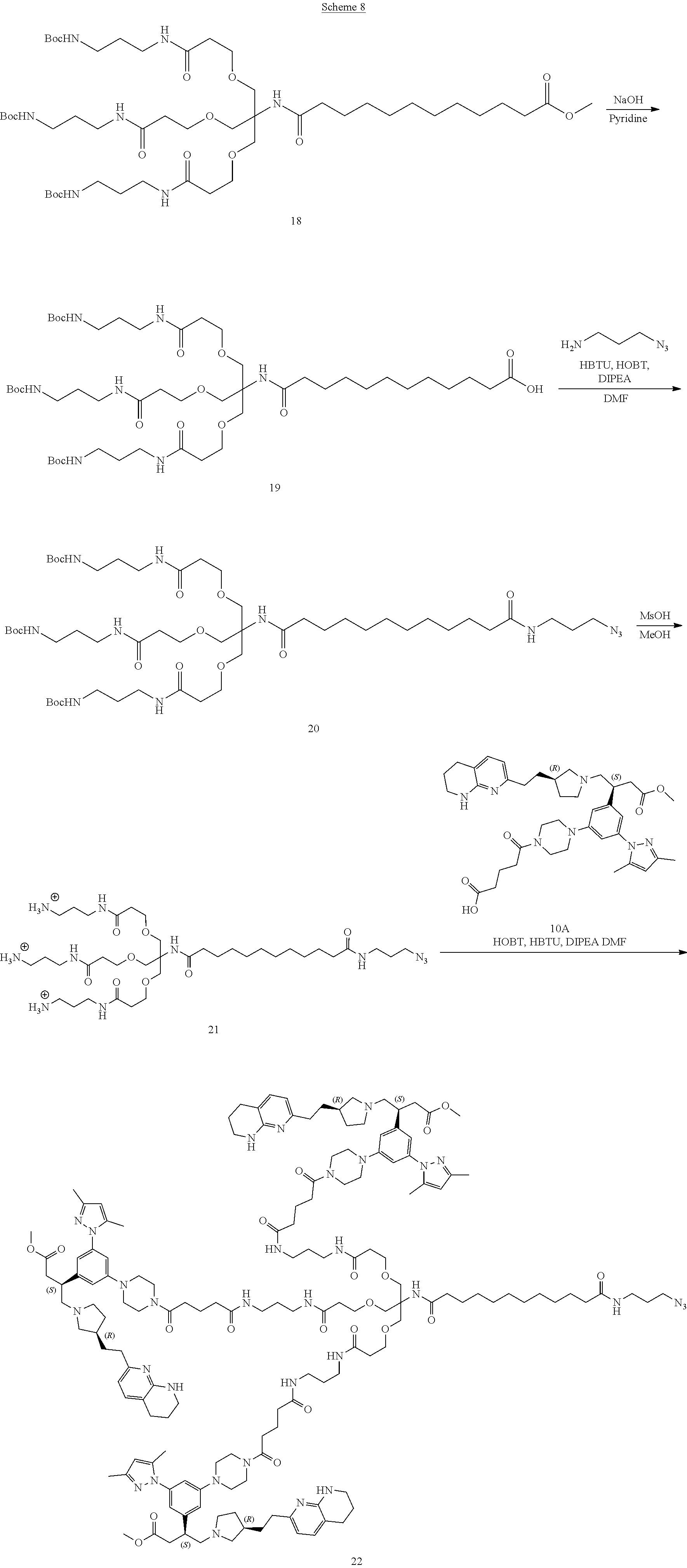

Compound 18 was synthesized by previously described methods (Carbohydrate conjugated ma agents and process for their preparation By: Nair, Jayaprakash K.; Kelin, Alexander V.; Kandasamy, Pachamuthu; Rajeev, Kallanthottahill G.; Manoharan, Muthiah United States, US20150196655 A1 2015-07-16)
Compound 19: Compound 18 (1.03 g, 1.0 mmol) is dissolved in pyridine (20 mL). NaOH (120 mg, 3 mmol) is added and the reaction mixture stir at room temperature for 18 hours. Triethylamine hydrochloride (413 mg, 3 mmol) was added, stirred for 5 minutes, then concentrated under vacuum to an oil. The crude oil was dissolved in ethyl acetate (40 mL) and washed with 22% NaCl (2×40 mL). The organic layer was dried over Na₂SO₄, filtered, and concentrated under vacuum to give compound 19, which was moved forward to the next step without further purification.
Compound 20: Compound 19 (1.02 g, 1.0 mmol), HOBt monohydrate (230 mg, 1.5 mmol) and HBTU (493 mg, 1.3 mmol) were dissolved in DMF (10 mL) under an inert atmosphere and cooled to 0° C. DIPEA (0.35 mL, 2.0 mmol) was added, and the reaction was allowed to stir for 5 minutes. A solution of 3-azido-1-propanamine (130 mg, 1.3 mmol) and DMF (5 mL) was added over a period of 10 minutes and the reaction allowed to stir at 0° C. for 4 hours. The reaction was diluted with ethyl acetate (50 mL) and washed with 5% NaCl (1×50 mL), 10% H₃PO₄, 5% NaCl (1×50 mL), 4% NaHCO₃ (1×50 mL), and satd. NaCl (1×50 mL), dried over Na₂SO₄, filtered, and concentrated under vacuum to give crude compound 20. The crude residue was purified by silica gel flash chromatography to give pure compound 20.
Compound 21: Compound 20 (1.10 g, 1.0 mmol) was dissolved in methanol (10 mL) under an inert atmosphere. Methanesulfonic acid (84 mL, 1.3 mmol) was added, and the reaction was heated to 75° C. and stirred for 18 hours. Triethylamine (0.042 mL, 0.3 mmol) was added, then the reaction was concentrated under reduced vacuum to give compound 21, which was moved forward to the next step without further purification.
Compound 22: Compound 10A (700 mg, 1.0 mmol), HOBt monohydrate (230 mg, 1.5 mmol) and HBTU (493 mg, 1.3 mmol) were dissolved in DMF (10 mL) under an inert atmosphere and cooled to 0° C. DIPEA (0.87 mL, 5.0 mmol) was added and the reaction was allowed to stir for 5 minutes. A solution of compound 21 (1.04 g, 1.3 mmol) in DMF (5 mL) was added over a period of 10 minutes and the reaction allowed to stir at 0° C. for 4 hours. The reaction was diluted with ethyl acetate (50) and washed with 5% NaCl (50 mL×1), 10% H₃PO₄, 5% NaCl (50 mL×1), 4% NaHCO₃ (50 mL×1), and satd. NaCl (50 mL×1), dried over Na₂SO₄, filtered, and concentrated under vacuum to give crude compound 22. The crude residue was purified by silica gel flash chromatography to give pure compound 22.
Compound 23: Compound 23 is synthesized in an analogous fashion to compound 22 starting with compounds 10B and 21.

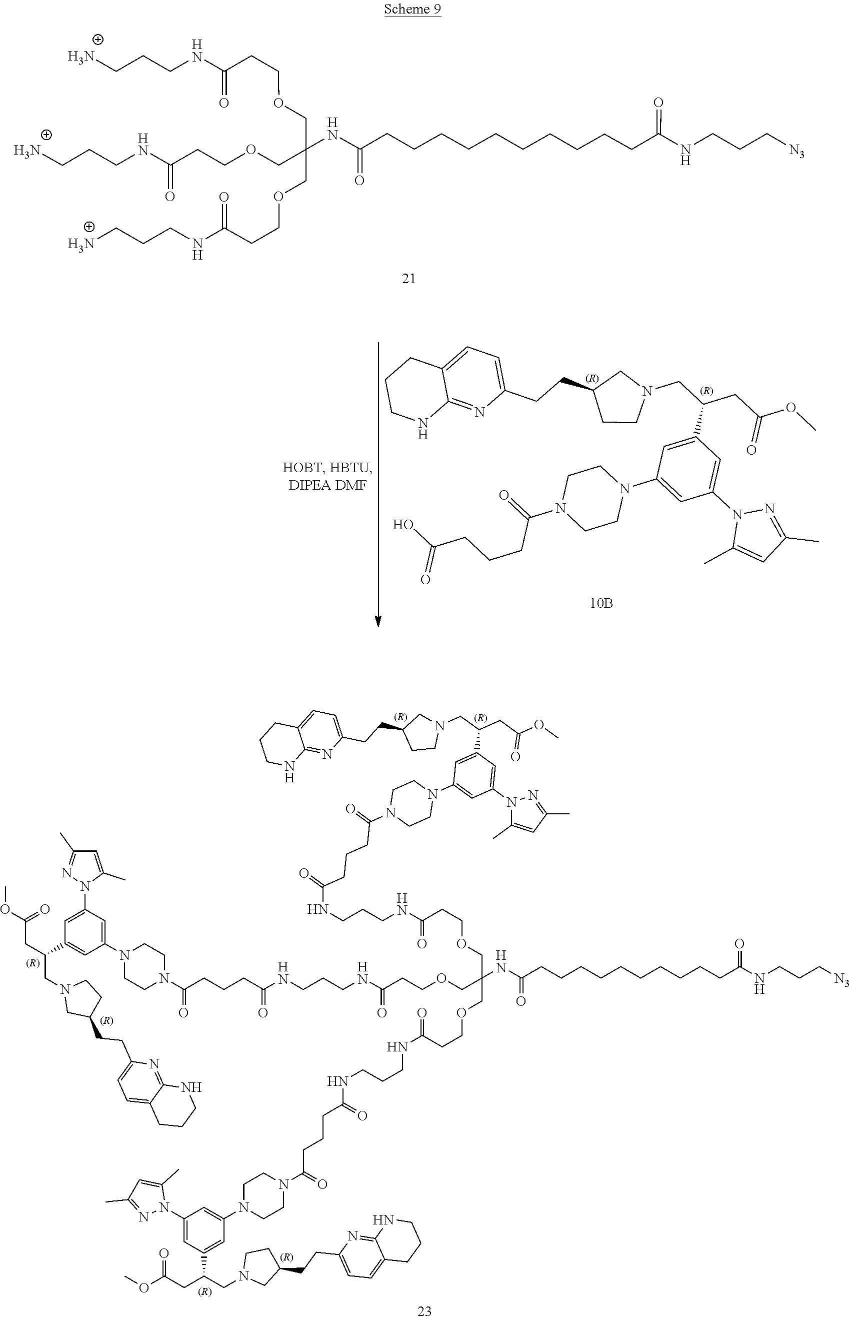

### Example 2: Design and Synthesis of αvβ6 Targeting Peptide Ligands

Peptide 1 was synthesized by Vivitide, LLC (Louisville, KY) according to the following sequence. H-Gly-Cys-Ile-Leu-Asn-Gly-Arg-Thr-Asp-Leu-Gly-Thr-Leu-Leu-Phe-Arg-Cus-Arg-Arg-Asp-Ser-Asp-Cys-Pro-Gly-Ala-Cys-Ile-Cys-Arg-Gly-Asn-Gly-Tyr-Cys-Gly-NH₂ (H-GCILNGRTDLGTLLFRCRRDSDCPGACICRGNGYCG-NH₂) (SEQ ID NO: 121) where disulfide bonds exist between C₂-C₂₇, C₁₇-C₂₉, C₂₃-C₃₅ that were formed by random oxidation with isolation of most thermodynamically stable isomer.

### Peptide 1:

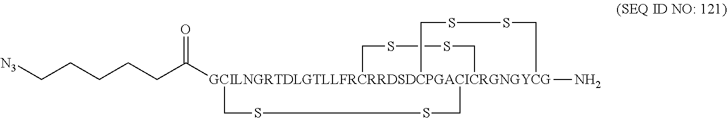

Peptide 2 was synthesized by Vivitide, LLC (Louisville, KY) according to the following sequence. cyclo[Phe-Arg-Gly-Asp-Leu-Ala-Phe-D-Pro-N-Me-Lys(PEG4-(CH₂)₅N₃)] (SEQ ID NO: 122)

### Peptide 2:

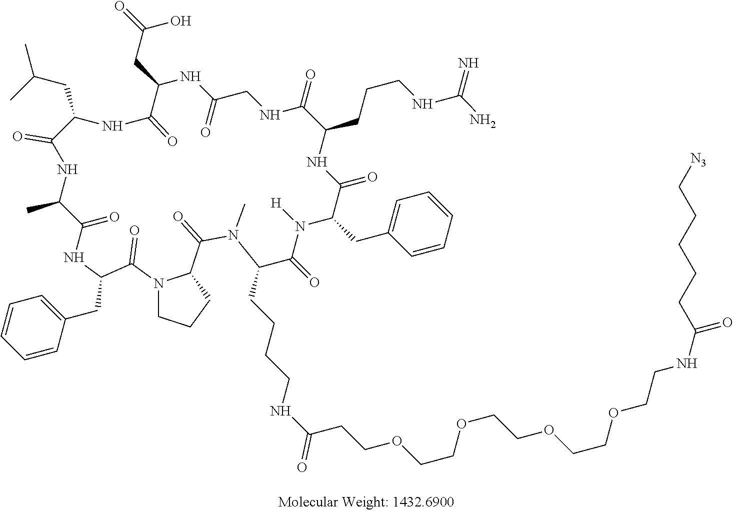 [SMILES: CC(C)C[C@@H]1NC(=O)[C@@H](CC(=O)O)NC(=O)CNC(=O)[C@@H](CCCNC(=N)N)NC(=O)[C@H](Cc2ccccc2)NC(=O)[C@H](CCCCNC(=O)CCOCCOCCOCCOCCNC(=O)CCCCCN=[N+]=[N-])N(C)C(=O)[C@@H]2CCCN2C(=O)[C@H](Cc2ccccc2)NC(=O)[C@@H](C)NC1=O]

Peptide 3 was synthesized by Vivitide, LLC (Louisville, KY) according to the following sequence. c(-K(azido)GPGRGDQTTLA-) (SEQ ID NO: 123)

### Peptide 3:

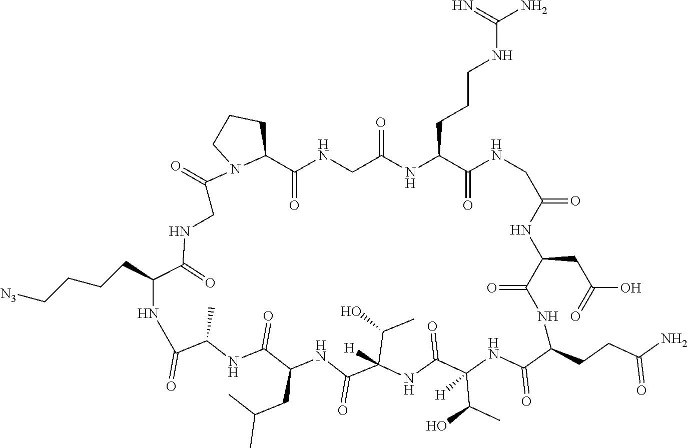 [SMILES: CC(C)C[C@@H]1NC(=O)[C@H]([C@@H](C)O)NC(=O)[C@H]([C@@H](C)O)NC(=O)[C@H](CCC(N)=O)NC(=O)[C@H](CC(=O)O)NC(=O)CNC(=O)[C@H](CCCNC(=N)N)NC(=O)CNC(=O)[C@@H]2CCCN2C(=O)CNC(=O)[C@H](CCCCN=[N+]=[N-])NC(=O)[C@H](C)NC1=O]

Peptide 4 was synthesized by Vivitide, LLC (Louisville, KY) according to the following sequence. c(-K(azido)AQGPGRGDQTTLAQA-) (SEQ ID NO: 124). Cyclic structure of peptide 4 is analogous to peptide 3

Peptide 5 was synthesized by Vivitide, LLC (Louisville, KY) according to the following sequence. c(-K(azido)QSAQGPGRGDQTTLAQAQA-) (SEQ ID NO: 125). Cyclic structure of peptide 5 is analogous to peptide 3

Peptide 6 was synthesized by Vivitide, LLC (Louisville, KY) according to the following sequence. c(-K(azido)NHQSAQGPGRGDQTTLAQAQTGA-) (SEQ ID NO: 126). Cyclic structure of peptide 6 is analogous to peptide 3

Peptide 7 was synthesized by Vivitide, LLC (Louisville, KY) according to the following sequence. c(-K(azido)ATNHQSAQGPGRGDQTTLAQAQTGWVA-) (SEQ ID NO: 127). Cyclic structure of peptide 7 is analogous to peptide 3

Peptide 8 was synthesized by Vivitide, LLC (Louisville, KY) according to the following sequence. c(-K(azido)QVATNHQSAQGPGRGDQTTLAQAQTGWVQNA-) (SEQ ID NO: 128). Cyclic structure of peptide 8 is analogous to peptide 3

Peptide 9 was synthesized by Vivitide, LLC (Louisville, KY) according to the following sequence. c(-K(azido)GPGAGAQTTLA-). (SEQ ID NO: 129) Cyclic structure of peptide 9 is analogous to peptide 3

Peptide 10 was synthesized by Vivitide, LLC (Louisville, KY) according to the following sequence. c(-K(azido)QVATNHQSAQGPGAGAQTTLAQAQTGWVQNA-) (SEQ ID NO: 130). Cyclic structure of peptide 10 is analogous to peptide 3

### Example 3: Design and Synthesis of αvβ6 Targeting siRNA Conjugates

This Example describes methods for the design, synthesis, and conjugation of siRNA agents. Nucleic acid sequences provided herein are represented using standard nomenclature. See the abbreviations of Table 1.

General Conditions for Precursors. Solid-Phase Oligonucleotide Synthesis:

Oligonucleotides were synthesized on a MerMade-12 DNA/RNA synthesizer. Sterling solvents/reagents from Glen Research, 500-Å controlled pore glass (CPG) solid supports from Prime Synthesis, 2′-deoxy nucleoside and 2′-O-TBDMS ribonucleoside 3′-phosphoramidites from Thermo, and 2′-OMe and 2′-F nucleoside 3′-phosphoramidites were all used as received. The 2′-OMe-uridine-5′-bis-POM-(E) vinyl phosphonate (VP) 3′-phosphoramidite was synthesized according to previously published procedures (Parmar et al, J Med Chem, 2018), dissolved to 0.15 M in 85% acetonitrile 15% dimethylformamide (DMF), and coupled using standard conditions on the synthesizer. Ligand-CPG support was prepared as described below. Low-water content acetonitrile was purchased from EMD Chemicals. A solution of 0.6 M 5-(S-ethylthio)-1H-tetrazole in acetonitrile was used as the activator. The phosphoramidite solutions were 0.15 M in anhydrous acetonitrile with 15% DMF as a co-solvent for 2′-OMe uridine and cytidine. The oxidizing reagent was 0.02 M I₂ in THF/pyridine/water. N,N-Dimethyl-N′-(3-thioxo-3H-1,2,4-dithiazol-5-yl)methanimidamide (DDTT), 0.09 M in pyridine, was used as the sulfurizing reagent. The detritylation reagent was 3% dichloroacetic acid (DCA) in dichloromethane (DCM).

After completion of the solid-phase synthesis, the CPG solid support was washed with 5% (v/v) piperidine in anhydrous acetonitrile three times with 5-min holds after each flow. The support was then washed with anhydrous acetonitrile and dried with argon. The oligonucleotides were then incubated with 28-30% (w/v) NH₄OH, at 35° C. for 20 h. For, VP-containing oligonucleotides, the CPG solid support was incubated with 28-30% (w/v) NH₄OH containing 5% (v/v) of diethylamine at 35° C. for 20 h (O'Shea et al, Tetrahedron, 2018). For amino containing oligonucleotides, the CPG solid support was incubated with a mixture of ammonia and methylamine (50%-50%, v/v, AMA) 35° C. for 3 h.

The solvent was collected by filtration, and the support was rinsed with water prior to analysis. After the filtration from the solid support, 2′-O-TBDMS RNA-containing oligonucleotides were subjected to a supplemental deprotection step, with adding triethylamine trihydrofluoride (37% HF) and additional incubation at 45° C. for 2 h. Oligonucleotide solutions of approximately 1 OD₂₆₀ units/mL were used for analysis of the crudes, and 30-50 μL of solution were injected. LC/ESI-MS was performed on an Agilent 6130 single quadrupole LC/MS system using an XBridge C8 column (2.1×50 mm, 2.5 μm) at 60° C. Buffer A consisted of 200 mM 1,1,1,3,3,3-hexafluoro-2-propanol and 16.3 mM triethylamine in water, and buffer B was 100% methanol. A gradient from 0% to 40% of buffer B over 10 min followed by washing and recalibration at a flow rate of 0.70 mL/min. The column temperature was 75° C. All oligonucleotides were purified and desalted, and further annealed to form siRNAs as previously described (Nair et al, JACS, 2014).

Conjugation of integrin ligands to the sense strand is carried out through various chemical reactions illustrated and described below. Sequences for sense and antisense strands can be found in Table 1.

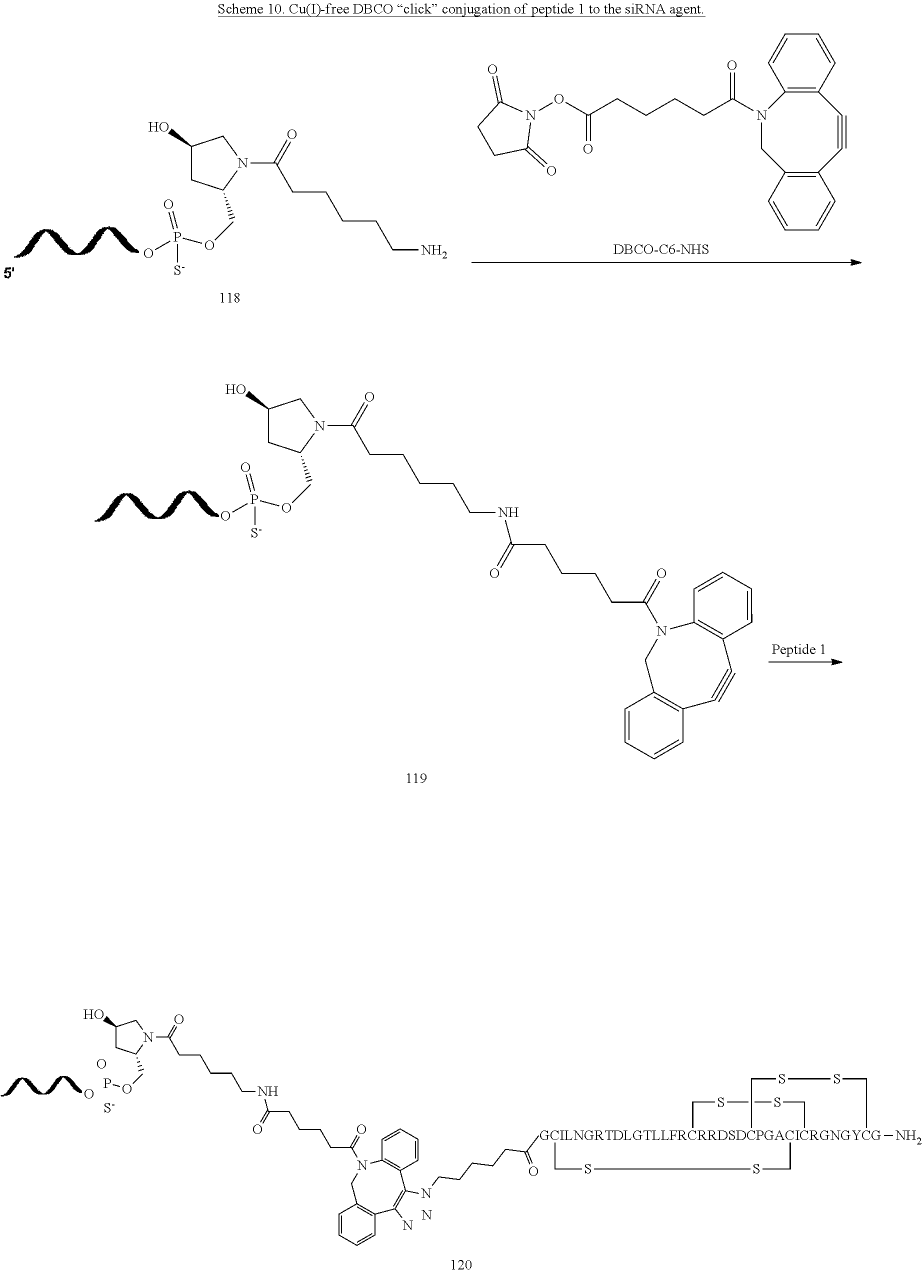

In a 20 mg scale synthesis, 1 equivalent of sense strand (118) containing 3′-amine was diluted to 1 mL with 1M pH 8.5 NaHCO₃ buffer. To this solution, 2 equivalents of (NHS-C6-DBCO), dissolved in 0.5 mL of anhydrous acetonitrile (ACN), was added. The reaction mixture was incubated at room temperature for 2 hours prior to desalting via size exclusion chromatography (SEC). Afterwards, the DBCO-modified sense strand (119) was resuspended with 1.5 mL of 0.5 M pH 7.5 HEPES buffer, followed by the addition of 1.5 equivalents of azide modified Peptide 1 in 1:1 NMP:1M pH 7.5 HEPES buffer. The resulting mixture was incubated at 37° C. for 2 hrs. Purification was performed using reverse phase chromatography (C₁₈ column) followed by desalting using SEC. [M] calculated for (120) 11410.764 Da, found 11410.82 Da (LC-MS). The purified sense strand conjugate 120 (19 mg, 95% yield, 97% purity via LCMS) was then lyophilized before annealing with the antisense strand. Conjugation of siRNA to azide modified Peptide 2 was performed in an analogous fashion as Peptide 1 to give Compound 121 (19 mg, 95% yield, 96% purity via LCMS, [M] calculated: 8873.904, found 8873.98 Da).

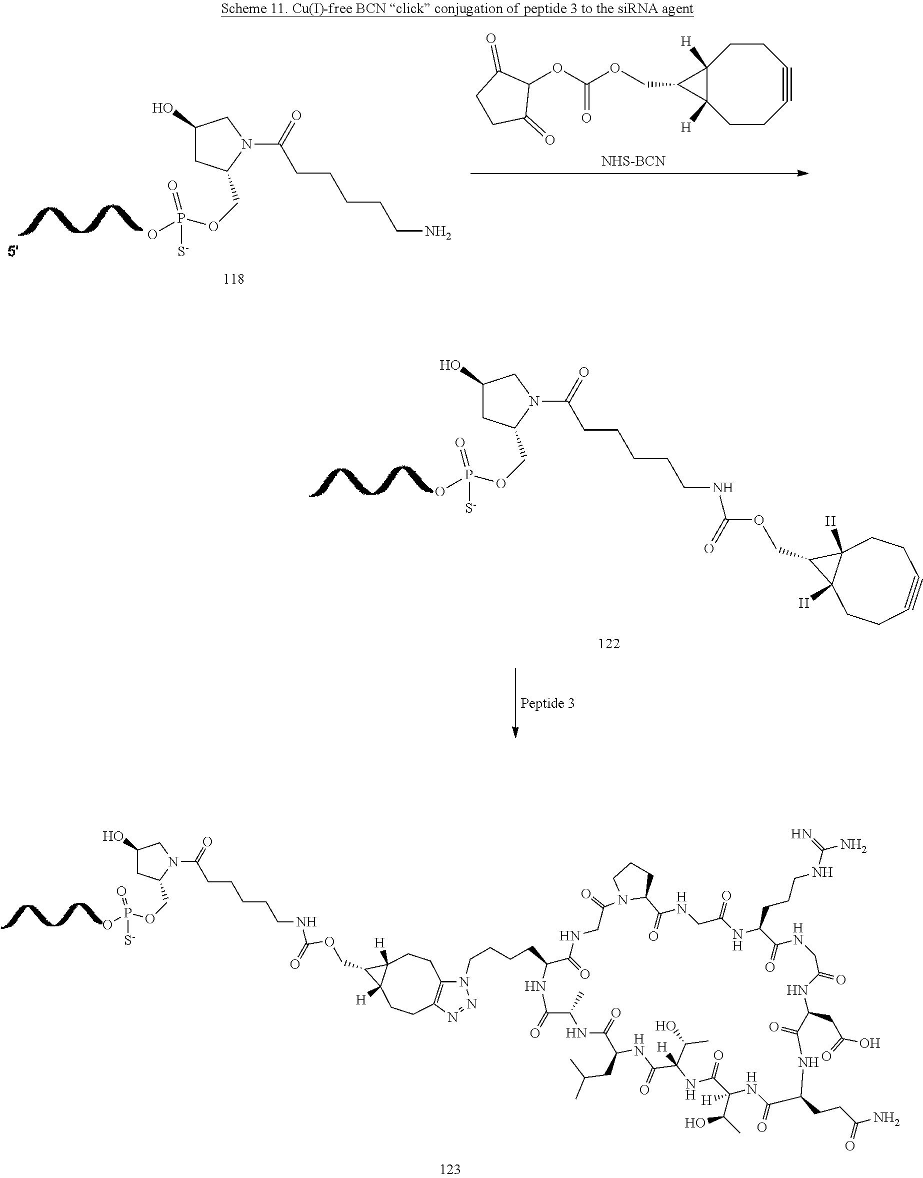 [SMILES: NCCCCCC(=O)N1C[C@H](O)C[C@H]1COP(=O)(O)[S-] ; O=C(OC[C@@H]1[C@@H]2CCC#CCC[C@@H]21)OC1C(=O)CCC1=O ; "NHS-BCN" ; O=C(NCCCCCC(=O)N1C[C@H](O)C[C@H]1COP(=O)(O)[S-])OC[C@@H]1[C@@H]2CCC#CCC[C@@H]21 ; CC(C)C[C@@H]1NC(=O)[C@H]([C@@H](C)O)NC(=O)[C@H]([C@@H](C)O)NC(=O)[C@H](CCC(N)=O)NC(=O)[C@H](CC(=O)O)NC(=O)CNC(=O)[C@H](CCCNC(=N)N)NC(=O)CNC(=O)[C@@H]2CCCN2C(=O)CNC(=O)[C@H](CCCCn2nnc3c2CC[C@@H]2[C@H](CC3)[C@@H]2COC(=O)NCCCCCC(=O)N2C[C@H](O)C[C@H]2COP(=O)(O)[S-])NC(=O)[C@H](C)NC1=O]

In a 40 mg scale synthesis, 1 equivalent of sense strand (118) containing 3′-amine was diluted to 1 mL with 1M pH 8.5 NaHCO₃ buffer. To this solution, 2 equivalents of N-[((1R,8S,9s)-Bicyclo[6.1.0]non-4-yn-9-yl)methyloxycarbonyloxy]succinimide (NHS-BCN), dissolved in 0.5 mL of anhydrous acetonitrile (ACN), was added. The reaction mixture was incubated at room temperature for 2 hours prior to desalting via size exclusion chromatography (SEC). Afterwards, the BCN-modified sense strand (122) was resuspended with 1.5 mL of 0.5 M pH 7.5 HEPES buffer, followed by the addition of 1.5 equivalents of azide-modified cRGD peptide 3 in 1:1 NMP:1M pH 7.5 HEPES buffer. The resulting mixture was incubated at 37° C. for 2 hrs. Purification was performed using reverse phase chromatography (C₁₈ column) followed by desalting using SEC. The purified sense strand conjugate 123 was then lyophilized before annealing with the antisense strand.

Conjugation of siRNA to azide-modified cRGD Peptides 4-10 are performed in an analogous fashion to Peptide 3 give Compounds 124-130.

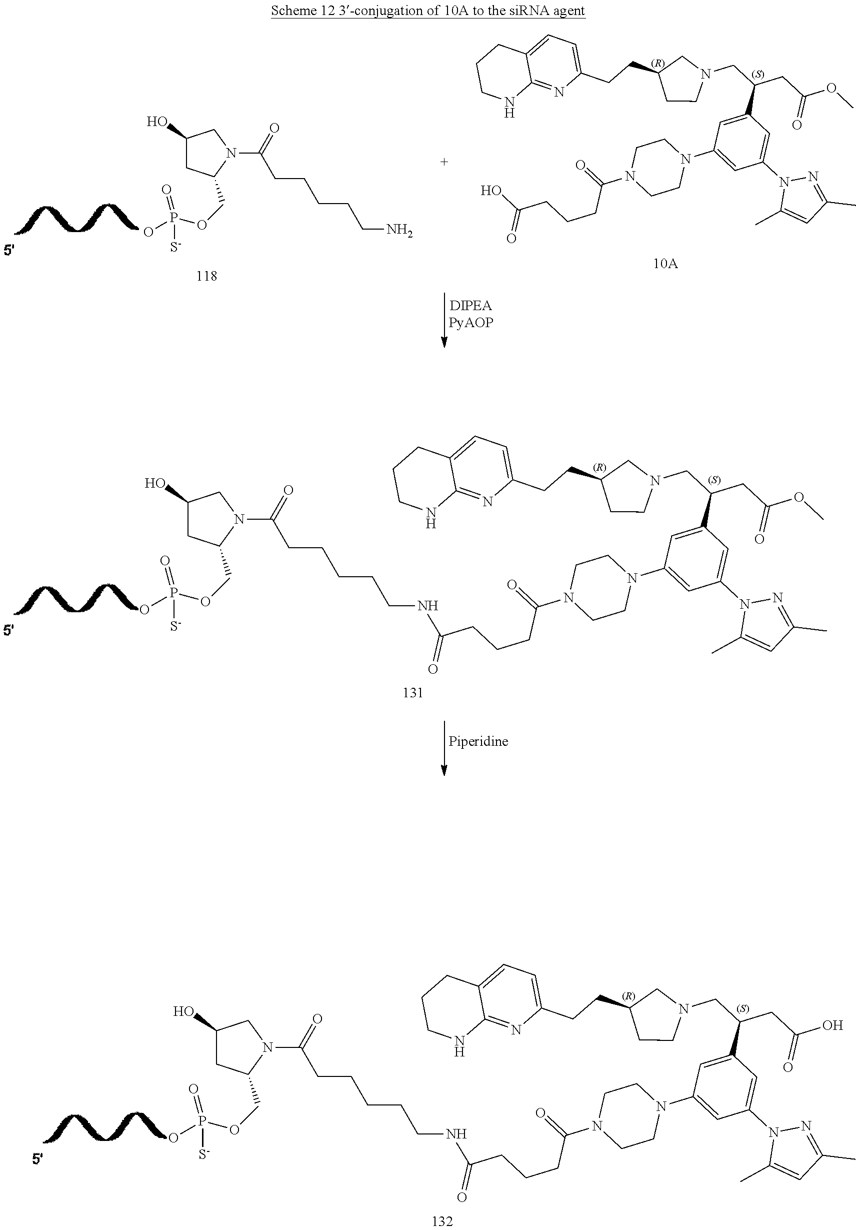

In a 50 mg scale synthesis, sense strand (118) was diluted to 1.25 mL with anhydrous NMP. The integrin ligand (10A) solution was prepared by combining 3 equivalents of (10A), 3 equivalents of 7-Azabenzotriazol-1-yloxy)tripyrrolidinophosphonium hexafluorophosphate (PyAOP) and 9 equivalents of N-Ethyldiisopropylamine (DIPEA) in anhydrous NMP. The two solutions were then mixed and incubated at room temperature for 5 minutes to afford (131). After the reaction reached completion, methyl ester deprotection was performed by adding 5 mL of 10% (v/v) aqueous piperidine to the crude mixture, followed by incubation at 40° C. for 16 hours. Purification was performed using reverse phase chromatography (C₁₈ column) followed by desalting using SEC. [M] calculated for (132) 7793.702 Da, found 7792.61 Da (LC-MS). The purified sense strand conjugate (41 mg, 84% yield, 98% purity via LCMS) was then lyophilized before annealing with the antisense strand.

Conjugation of siRNA to integrin ligands 461, 462, and 454 is performed in an analogous fashion to Compound 132 give Compounds 461B, 462B, and 454B.

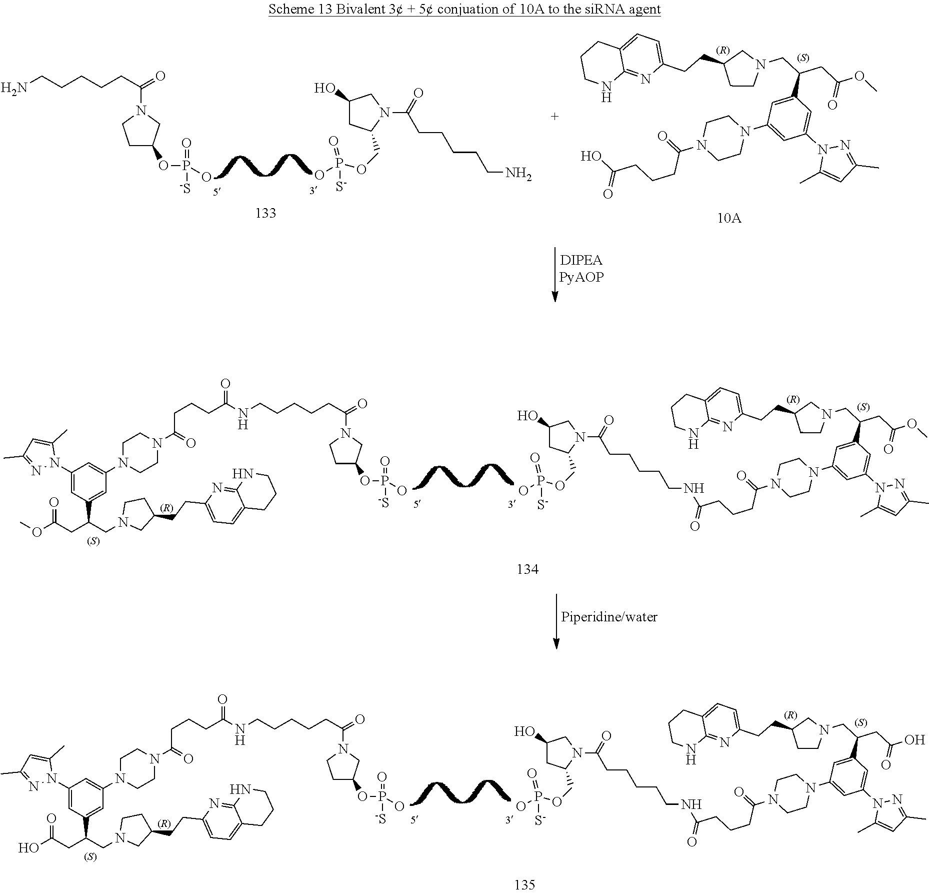

In a 50 mg scale synthesis, sense strand (133) was diluted to 1.25 mL with anhydrous NMP. The integrin ligand (1LA) solution was prepared by combining 6 equivalents of (10A), 6 equivalents of 7-Azabenzotriazol-1-yloxy)tripyrrolidinophosphonium hexafluorophosphate (PyAOP) and 18 equivalents of N-Ethyldiisopropylamine (DIPEA) in anhydrous NMP. The two solutions were then mixed and incubated at room temperature for 5 minutes to afford (134). After the reaction reached completion, methyl ester deprotection was performed by adding 5 mL of 10% (v/v) aqueous piperidine to the crude mixture, followed by incubation at 40° C. for 16 hours. Purification was performed using reverse phase chromatography (C₁₈ column) followed by desalting using SEC. [M] calculated for (135) 8724.808 Da, found 8724.01 Da (LC-MS). The purified sense strand conjugate (33 mg, 66% o yield, 99% o purity via LCMS) was then lyophilized before annealing with the antisense strand.

Conjugation of siRNA to integrin ligand 10B was performed in an analogous fashion as 10A to give Compound 136 (32 mg, 64% yield, 99% purity, [M] calculated: 8724.808, found 8724.02).

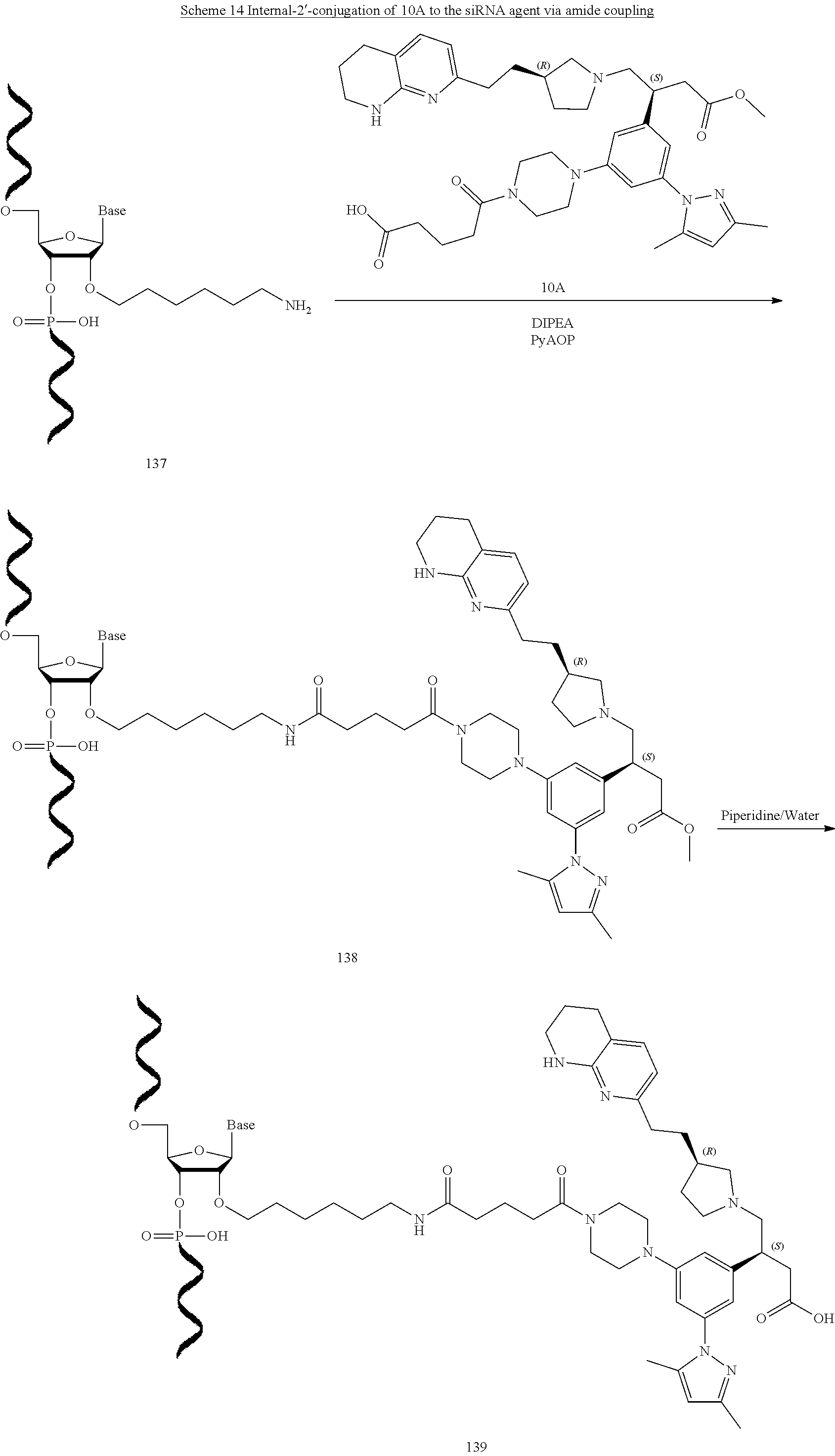

In a 50 mg scale synthesis, sense strand (137) was diluted to 1.25 mL with anhydrous NMP. The integrin ligand (10A) solution was prepared by combining 3 equivalents of (10A), 3 equivalents of 7-Azabenzotriazol-1-yloxy)tripyrrolidinophosphonium hexafluorophosphate (PyAOP) and 9 equivalents of N-Ethyldiisopropylamine (DIPEA) in anhydrous NMP. The two solutions were then mixed and incubated at room temperature for 5 minutes to afford (138). After the reaction reached completion, methyl ester deprotection was performed by adding 5 mL of 10% (v/v) aqueous piperidine to the crude mixture, followed by incubation at 40° C. for 16 hours. Purification was performed using reverse phase chromatography (C₁₈ column) followed by desalting using SEC. The purified sense strand conjugate 139 was then lyophilized before annealing with the antisense strand.

Conjugation of siRNA to integrin ligand 10B was performed in an analogous fashion as 10A to give Compound 140

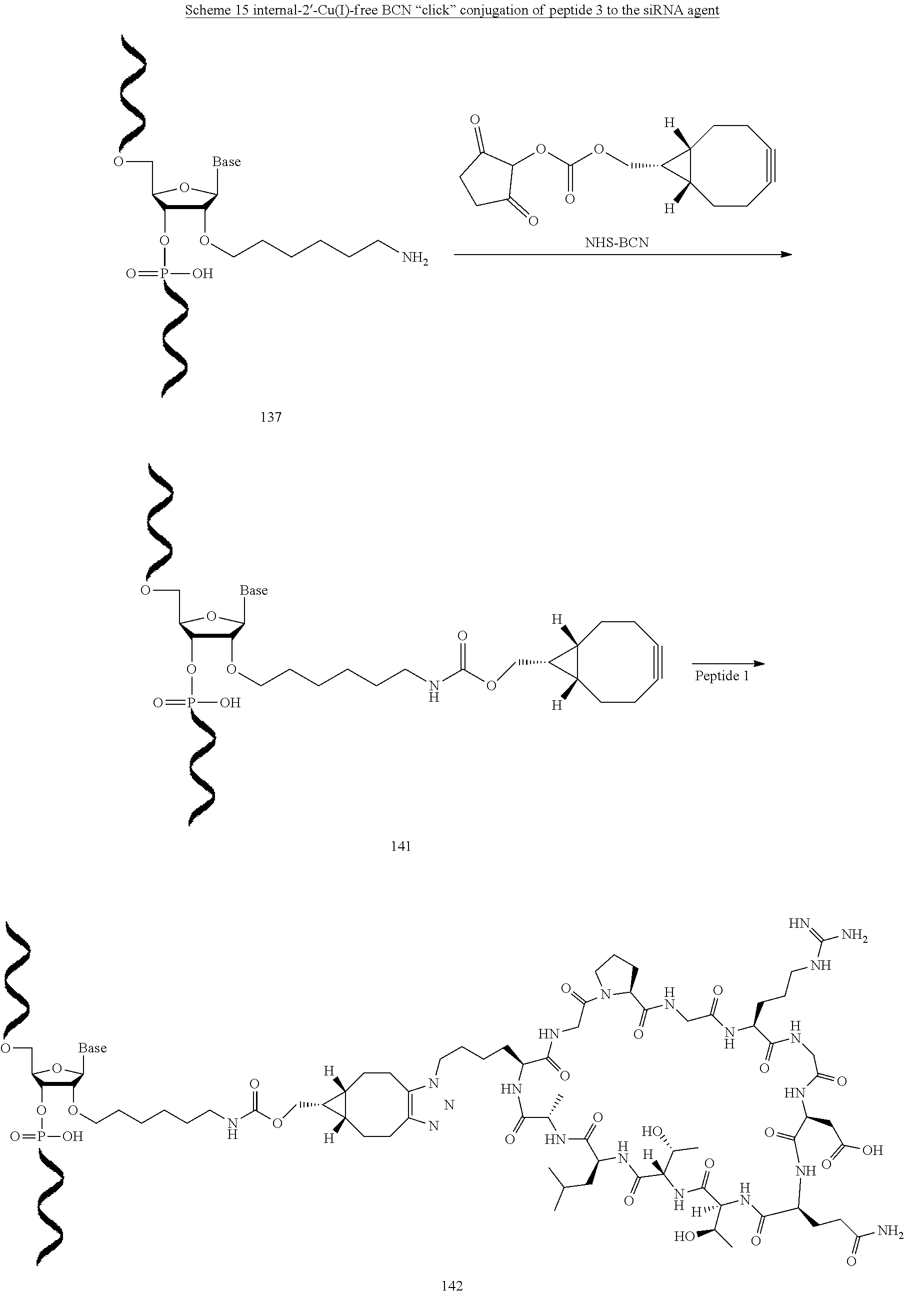

In a 40 mg scale synthesis, 1 equivalent of sense strand (137) containing 3′-amine was diluted to 1 mL with 1M pH 8.5 NaHCO₃ buffer. To this solution, 2 equivalents of N-[((1R,8S,9s)-Bicyclo[6.1.0]non-4-yn-9-yl)methyloxycarbonyloxy]succinimide (NHS-BCN), dissolved in 0.5 mL of anhydrous acetonitrile (ACN), was added. The reaction mixture was incubated at room temperature for 2 hours prior to desalting via size exclusion chromatography (SEC). Afterwards, the BCN-modified sense strand (141) was resuspended with 1.5 mL of 0.5 M pH 7.5 HEPES buffer, followed by the addition of 1.5 equivalents of azide-modified cRGD peptide 1 in 1:1 NMP:1M pH 7.5 HEPES buffer. The resulting mixture was incubated at 37° C. for 2 hrs. Purification was performed using reverse phase chromatography (C₁₈ column) followed by desalting using SEC. The purified sense strand conjugate 142 was then lyophilized before annealing with the antisense strand.

Conjugation of siRNA to azide-modified cRGD Peptides 2-10 is performed in an analogous fashion to Peptide 1 give Compounds 143-149

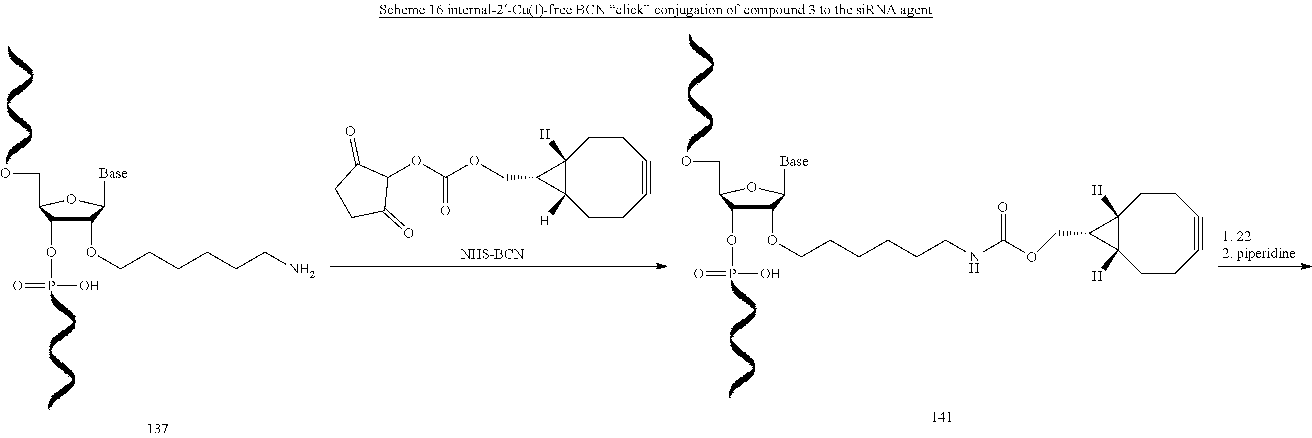

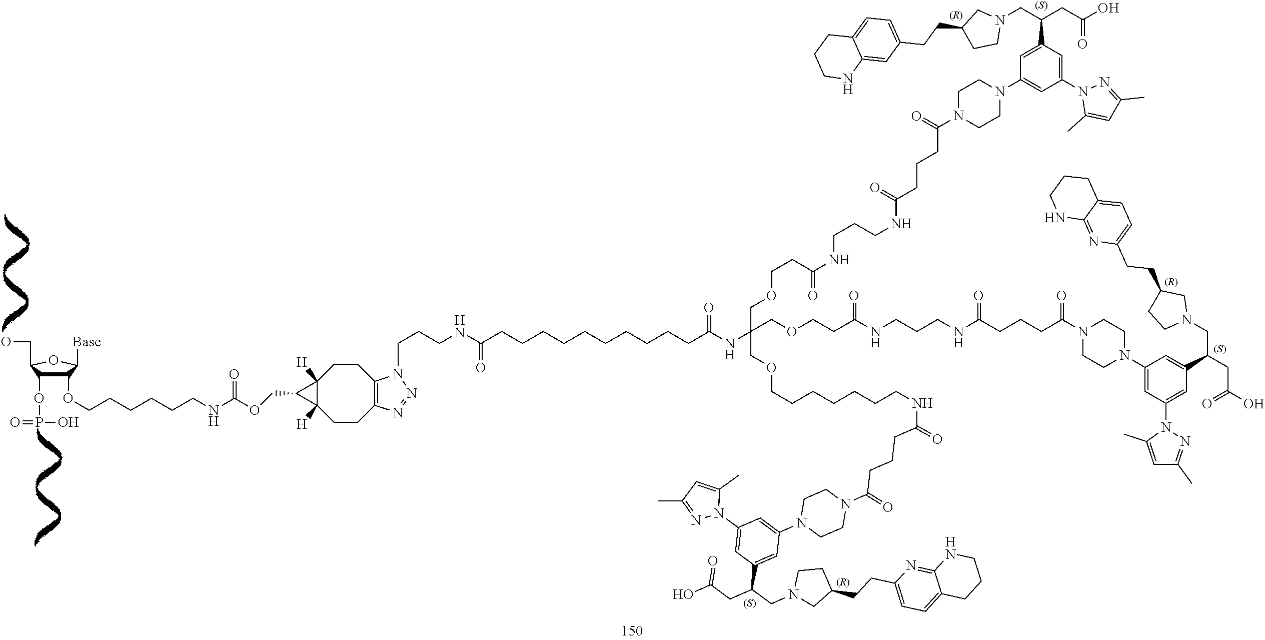

In a 40 mg scale synthesis, 1 equivalent of sense strand (137) containing 3′-amine was diluted to 1 mL with 1M pH 8.5 NaHCO₃ buffer. To this solution, 2 equivalents of N-[((1R,8S,9s)-Bicyclo[6.1.0]non-4-yn-9-yl)methyloxycarbonyloxy]succinimide (NHS-BCN), dissolved in 0.5 mL of anhydrous acetonitrile (ACN), was added. The reaction mixture was incubated at room temperature for 2 hours prior to desalting via size exclusion chromatography (SEC). Afterwards, the BCN-modified sense strand (141) was resuspended with 1.5 mL of 0.5 M pH 7.5 HEPES buffer, followed by the addition of 1.5 equivalents of compound 22 in 1:1 NMP:1M pH 7.5 HEPES buffer. The resulting mixture was incubated at 37° C. for 2 hrs. After the reaction reached completion, methyl ester deprotection was performed by adding 5 mL of 10% (v/v) aqueous piperidine to the crude mixture, followed by incubation at 40° C. for 16 hours. Purification was performed using reverse phase chromatography (C₁₈ column) followed by desalting using SEC. The purified sense strand conjugate 150 was then lyophilized before annealing with the antisense strand.

Conjugation of siRNA to compound 23 is performed in an analogous fashion to compound 22 to give Compound 151.

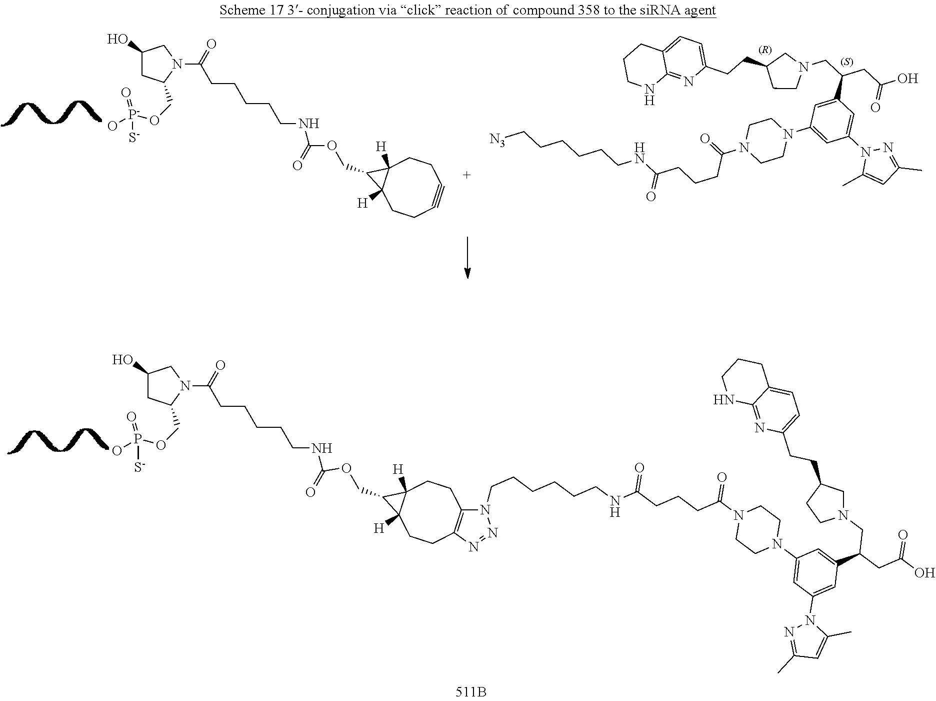

In a 40 mg scale synthesis, 1 equivalent of sense strand (133) containing 3′-amine was diluted to 1 mL with 1 M pH 8.5 NaHCO₃ buffer. To this solution, 2 equivalents of N-[((1R,8S,9s)-Bicyclo[6.1.0]non-4-yn-9-yl)methyloxycarbonyloxy]succinimide (NHS-BCN), dissolved in 0.5 mL of anhydrous acetonitrile (ACN), was added. The reaction mixture was incubated at room temperature for 2 hours prior to desalting via size exclusion chromatography (SEC). Afterwards, the 3′-BCN-modified sense strand (122) was resuspended with 1.5 mL of 0.5 M pH 7.5 HEPES buffer, followed by the addition of 1.5 equivalents of azide ligand (511) in 1:1 NMP:1 M pH 7.5 HEPES buffer. The resulting mixture was incubated at 37° C. for 2 hrs. Purification was performed using reverse phase chromatography (C₁₈ column) followed by desalting using SEC. The purified sense strand conjugate was then lyophilized before annealing with the antisense strand.

Conjugation of siRNA to compounds 512-517 is performed in an analogous fashion to compound 511 to give Compounds 512B-517B.

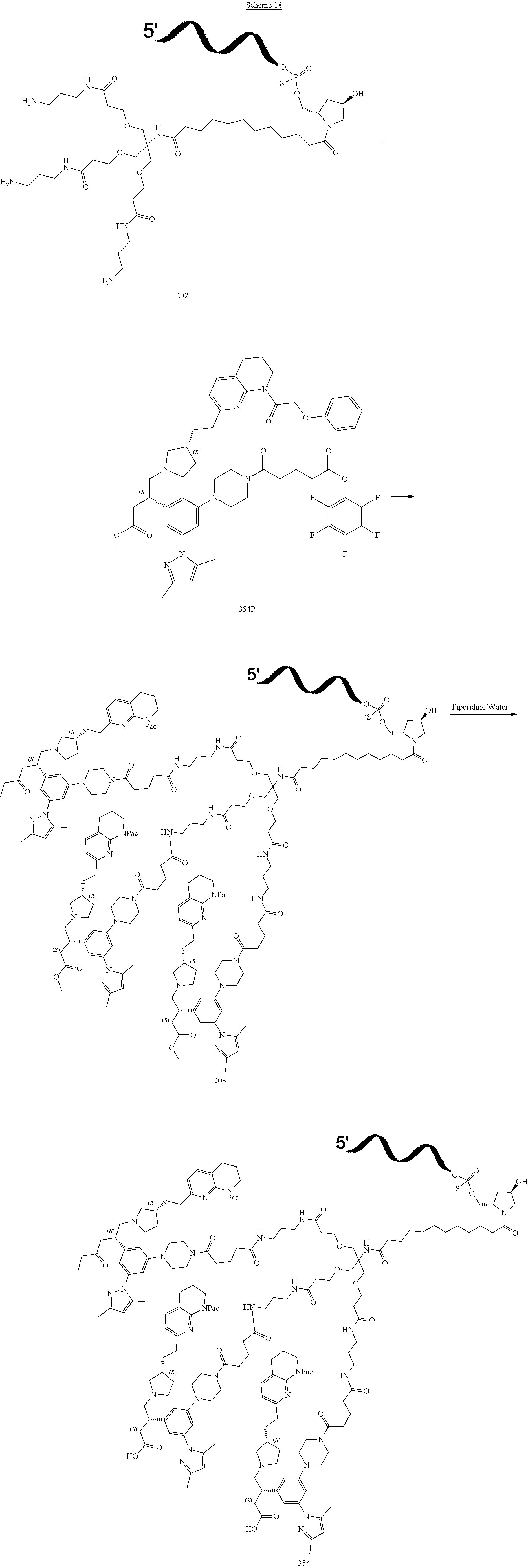

In a 40 mg scale synthesis, 1 equivalent of sense strand (202) containing 3′-triamine was diluted to 50-100 mM with 1 M pH 8.5 NaHCO₃ buffer. To this solution, 12 equivalents (3 equiv per amino group) of 354P dissolved in 0.5 mL of NMP, was added. The reaction mixture was incubated at room temperature for 12 hours to afford (203). After the reaction reached completion, methyl ester deprotection was performed by adding 5 mL of 10% (v/v) aqueous piperidine to the crude mixture, followed by incubation at 40° C. for 16 hours. Purification was performed using reverse phase chromatography (C₁₈ column) followed by desalting using SEC. The purified sense strand conjugate was then lyophilized before annealing with the antisense strand.

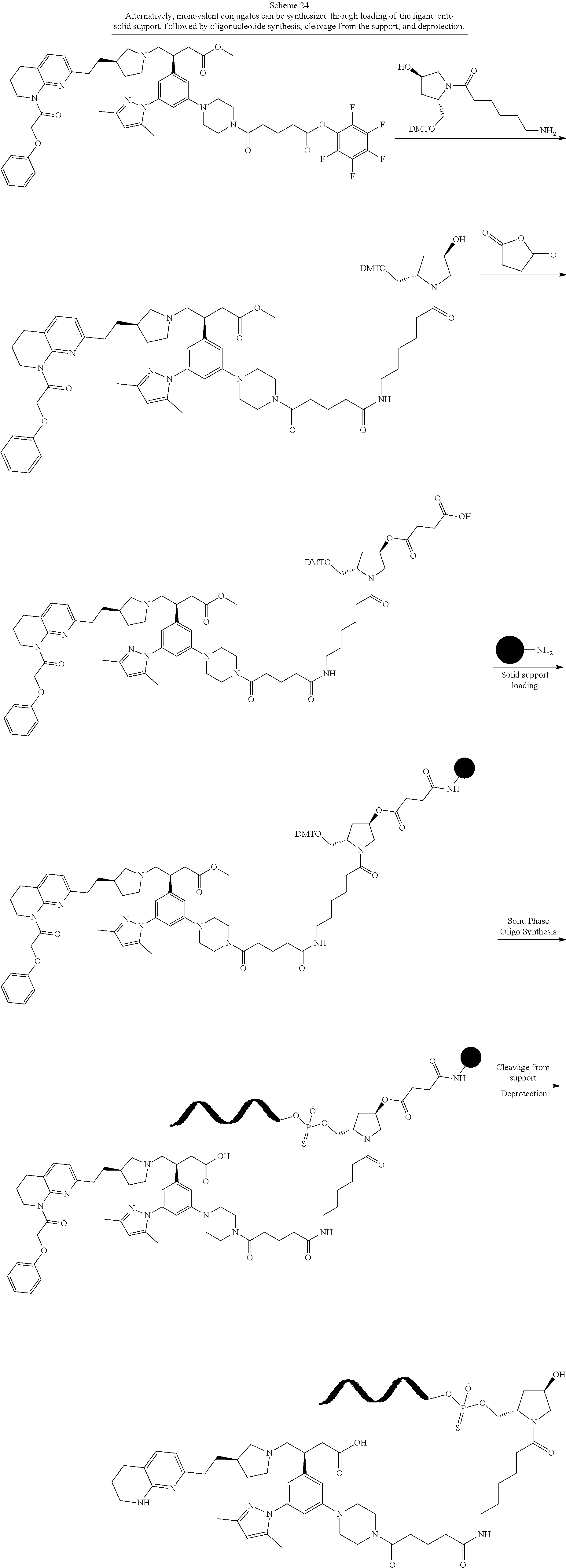

A mixture of PFP-ester intermediate (15 g, 15 mmol) and primary amine intermediate (7.61 g, 14.28 mmol) was dissolved in DCM (80 mL). After the solution was stirred at rt for 23 h the solvent was removed under vacuum. The crude residue (25.3 g) was solid loaded onto a 330 g silica column that had been equilibrated with DCM containing 1% TEA. The column was eluted with a 0-10% MeOH/DCM gradient. Pure fractions were pulled and dried affording 15.2 g (78.9%) of pure amide product as a yellow solid. 1H NMR (600 MHz, DMSO) δ 7.75 (t, J=5.5 Hz, 1H), 7.52 (d, J=7.6 Hz, 1H), 7.33-7.26 (m, 5H), 7.25-7.22 (m, 2H), 7.21-7.14 (m, 5H), 6.97 (d, J=7.6 Hz, 1H), 6.89 (d, J=7.3 Hz, 2H), 6.88-6.84 (m, 4H), 6.82 (s, 1H), 6.78 (d, J=7.7 Hz, 3H), 6.71 (s, 1H), 6.02 (s, 1H), 5.22 (s, 2H), 4.94 (d, 1H), 4.33 (d, 1H), 4.18-4.06 (m, 1H), 3.78 (t, 2H), 3.72 (s, 6H), 3.62-3.55 (m, 3H), 3.54 (s, 2H), 3.46 (s, 3H), 3.20-3.13 (m, 4H), 3.12 (t, J=5.3 Hz, 2H), 3.03-2.96 (m, 3H), 2.78 (dd, J=15.5, 6.2 Hz, 1H), 2.73 (t, J=6.4 Hz, 2H), 2.67 (t, J=9.3 Hz, 2H), 2.63-2.60 (m, 2H), 2.57 (d, J=7.2 Hz, 1H), 2.53 (d, J=8.4 Hz, 1H), 2.39 (t, J=11.6 Hz, 1H), 2.35 (s, 1H), 2.32 (t, J=7.5 Hz, 2H), 2.23 (s, 3H), 2.20 (t, J=7.5 Hz, 1H), 2.15 (s, 3H), 2.13 (d, J=8.2 Hz, 1H), 2.11-2.07 (m, 2H), 2.00 (dq, J=14.3, 6.8 Hz, 2H), 1.96-1.88 (m, 1H), 1.84 (m, 4H), 1.73 (q, J=7.5 Hz, 2H), 1.66 (dd, J=7.6, 2.8 Hz, 2H), 1.47 (m, 2H), 1.38 (m, 2H), 1.35-1.19 (m, 4H), 1.18-1.01 (m, 1H). MS (ESI+APCI), calculated for C₇₉H₉₈N₉O₁ [M+H]⁺ exact mass m/z=1348.74, found 1350.2.

A solution of amide intermediate (1.0 g, 741.47 mol), succinic anhydride (222.60 mg, 2.22 mmol), and dimethylaminopyridine (272 mg, 2.22 mmol) in DCM (5.7 mL) was stirred at rt for 16 h. The reaction was diluted with DCM (50 mL), washed with 0.1 M triethylammonium bicarbonate buffer (3×50 mL), dried over Na₂SO₄, and dried under vacuum. The crude residue (0.95 g) was taken up in DCM and injected onto a 12 g silica column that had been equilibrated with 5% TEA in DCM. The column was eluted with a 0-20% MeOH/DCM gradient. Pure fractions were pulled and dried affording 1.02 g (88.8%) of the mono-triethylamine salt of succinate adduct product as an off white solid. 1H NMR (600 MHz, DMSO) δ 7.76 (t, J=5.9 Hz, 1H), 7.52 (d, J=7.6 Hz, 1H), 7.30 (dq, J=15.6, 7.5 Hz, 4H), 7.23 (d, J=7.9 Hz, 2H), 7.22-7.15 (m, 5H), 6.97 (d, J=7.6 Hz, 1H), 6.92-6.84 (m, 5H), 6.82 (s, 1H), 6.78 (d, J=8.7 Hz, 3H), 6.71 (s, 1H), 6.01 (s, 1H), 5.76 (s, 8H), 5.35 (s, 1H), 5.31 (s, 3H), 5.22 (s, 2H), 4.18 (s, 1H), 3.80-3.77 (m, 2H), 3.72 (s, 6H), 3.57 (s, 2H), 3.56-3.52 (m, 3H), 3.46 (s, 4H), 3.37 (q, J=7.3 Hz, 13H), 3.16 (s, 3H), 3.12 (s, 2H), 3.04-2.98 (m, 3H), 2.82-2.71 (m, 9H), 2.67 (t, J=8.4 Hz, 2H), 2.61 (td, J=8.2, 2.8 Hz, 2H), 2.57 (d, J=7.4 Hz, 1H), 2.53 (d, J=8.4 Hz, 1H), 2.47 (q, J=3.4 Hz, 2H), 2.45 (d, J=2.9 Hz, 1H), 2.41-2.36 (m, 1H), 2.32 (q, J=6.9 Hz, 3H), 2.23 (s, 3H), 2.20 (dd, J=13.4, 5.5 Hz, 2H), 2.15 (s, 3H), 2.10 (dt, J=10.7, 7.5 Hz, 3H), 2.05-1.93 (m, 2H), 1.91 (s, 2H), 1.84 (m, 3H), 1.73 (q, J=7.4 Hz, 2H), 1.70-1.62 (m, 2H), 1.46 (q, J=10.1 Hz, 2H), 1.39 (m, 2H), 1.33-1.25 (m, 3H), 1.23 (t, J=7.2 Hz, 14H), 1.06 (t, J=7.2 Hz, 8H). MS (ESI+APCI), calculated for C₈₃H₁₀₂N₉O₁₄ [M+H]⁺ exact mass m/z=1448.75, found 1448.6.

A mixture of the mono-triethylamine salt of succinate adduct (0.50 g, 345 mol) and DIPEA (0.45 g, 3.45 mmol, 600 L) was dissolved in ACN (250 mL) in a round-bottom flask with a reinforced neck. To this solution was added HBTU (157 mg, 414 mol) and the reaction was stirred for ˜10 min. To the reaction mixture was added CPG (3.451 g, 153 mol/g). The reaction was put on the mechanical shaker for 16 h. The mixture was filtered, washed with ACN (200 mL), MeOH (200 mL), ACN (200 mL), diethyl ether (200 mL) and dried on the high vacuum for 4 h. The CPG was transferred to a round-bottom flask with a reinforced neck and a 1:1 mixture of cap A & B (400 mL) was added. (Cap Mix A=5% Phenoxyacetic Anhydride in THF/Pyridine Glen Research pn #40-4210-52) (Cap Mix B=10% 1-Methylimidazole in THF Glen Research pn #40-4120-57). This mixture was put back on the mechanical shaker for 3 h. The reaction mixture was filtered, and the obtained CPG was washed with 10% H₂O/THF (100 mL), MeOH (100 mL), 10% H₂O/THF (100 mL), MeOH (100 mL), ACN (100 mL), and diethyl ether (100 mL) then dried on the high vacuum for 60 h affording 3.22 g of loaded product with a loading of 58.53 mol/g.

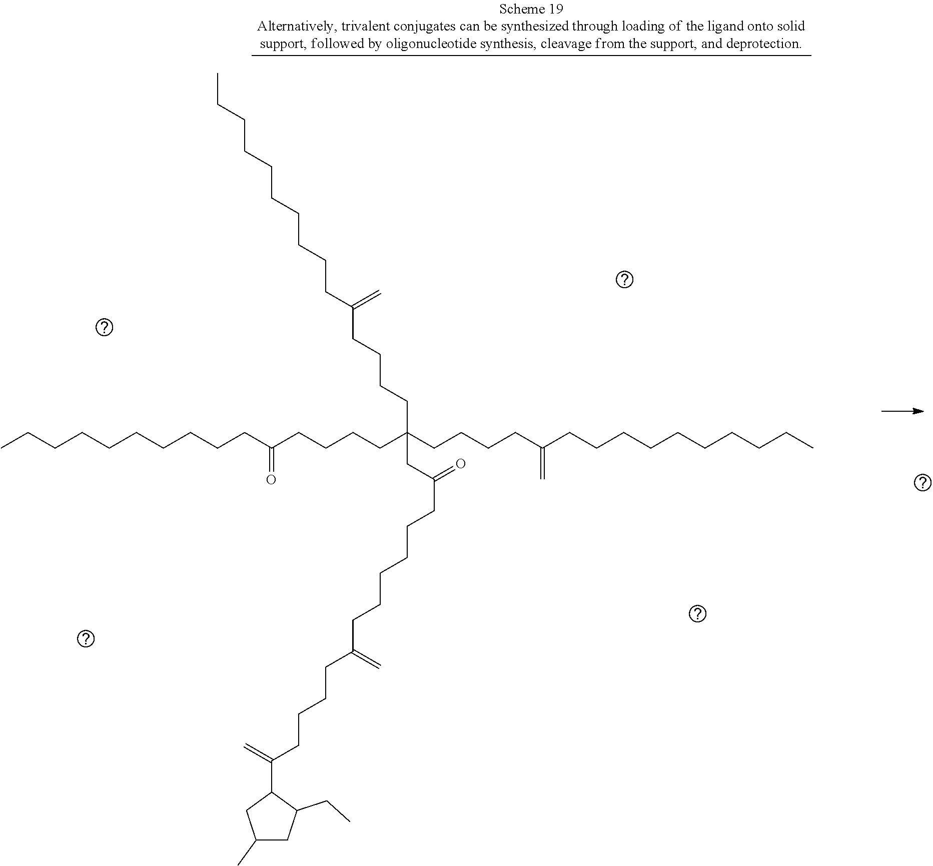 [SMILES: C=C(CCCCCCC(=O)CC(CCCCC(=C)CCCCCCCCCC)(CCCCC(=C)CCCCCCCCCC)CCCCC(=O)CCCCCCCCCC)CCCCC(=C)C1CC(C)CC1CC]

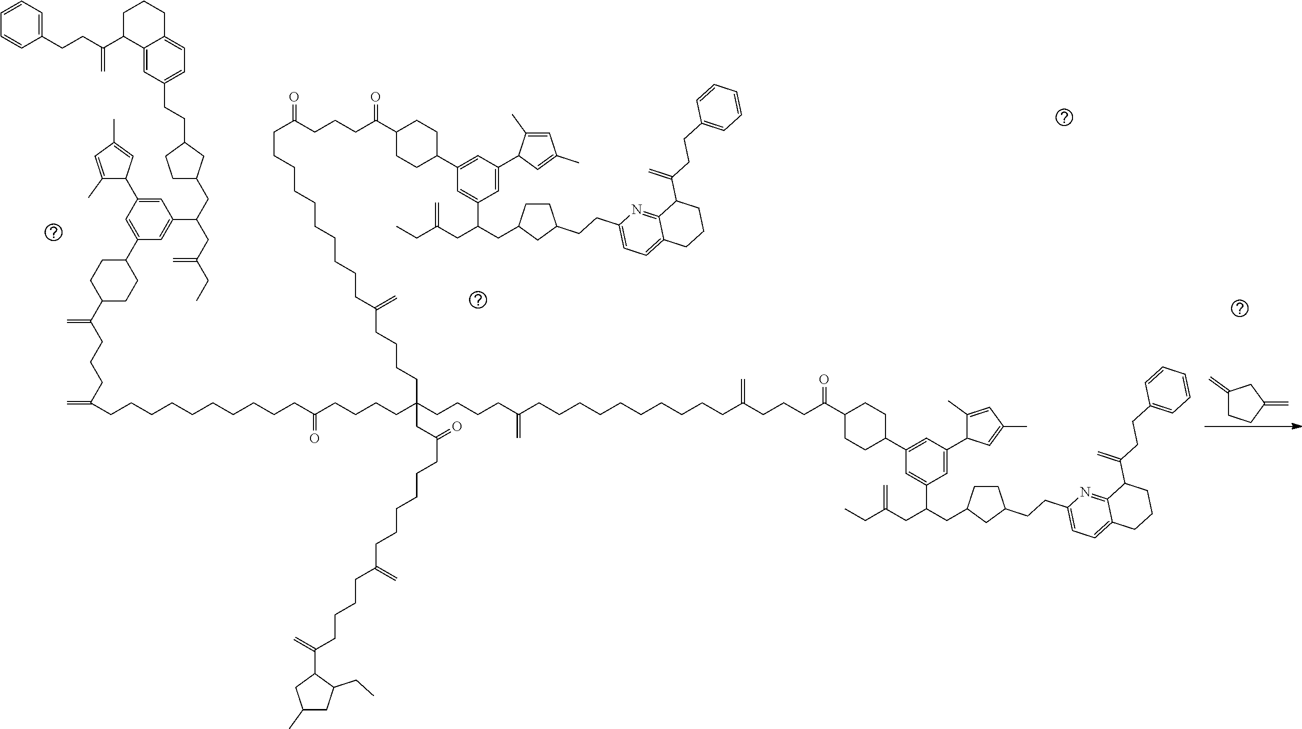

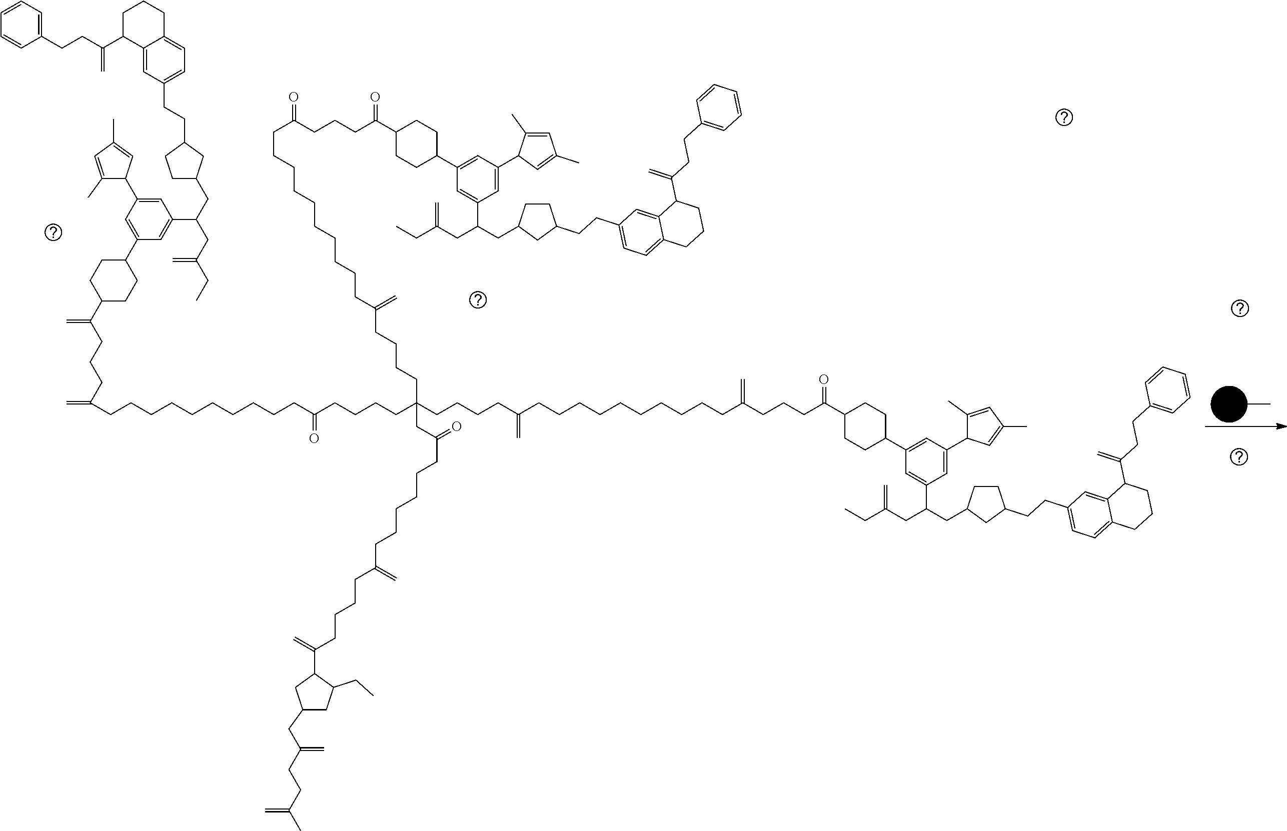

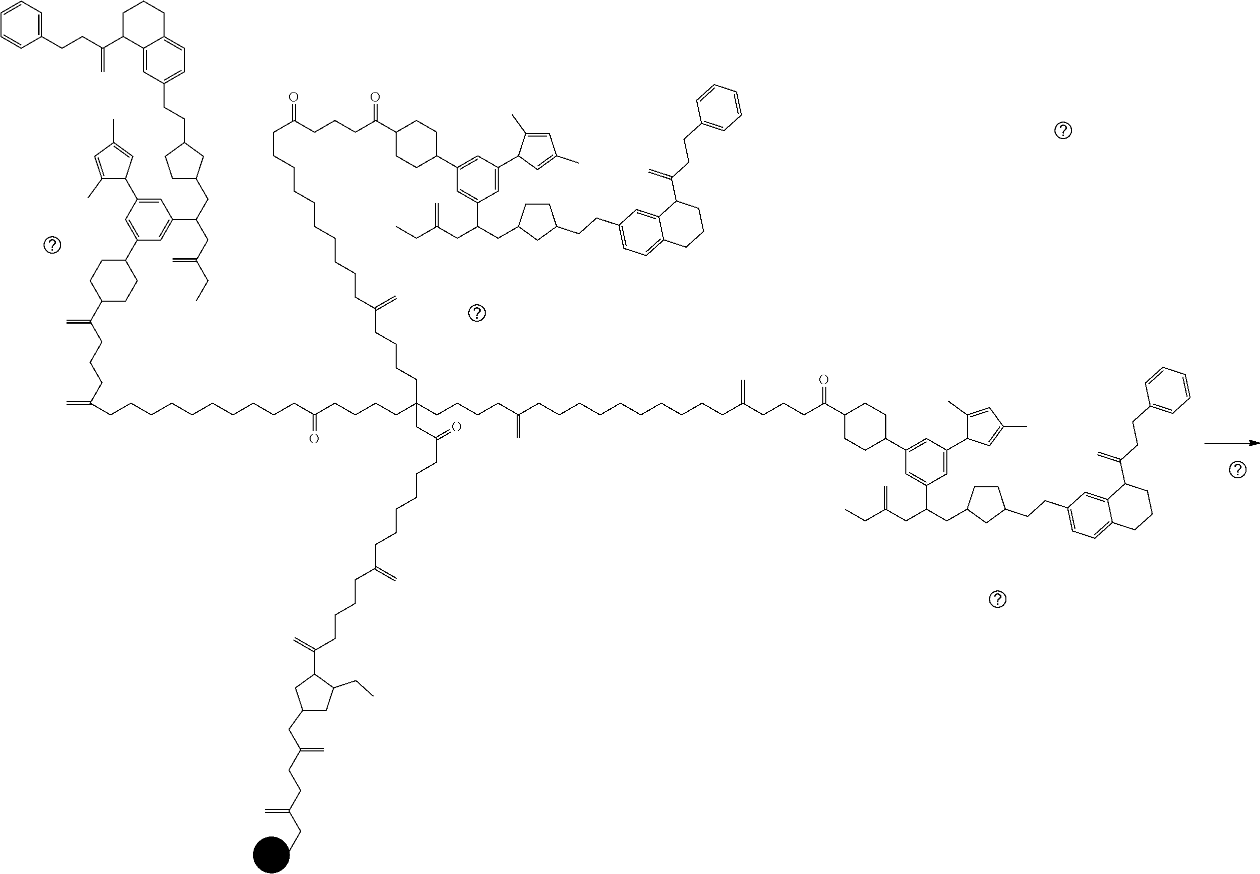 [SMILES: C=C(CC)CCC(=C)CC1CC(CC)C(C(=C)CCCCC(=C)CCCCCCC(=O)CC(CCCCC(=C)CCCCCCCCCCC(=C)CCCC(=O)C2CCC(c3cc(C(CC(=C)CC)CC4CCC(CCc5ccc6c(c5)C(C(=C)CCc5ccccc5)CCC6)C4)cc(C4C=C(C)C=C4C)c3)CC2)(CCCCC(=C)CCCCCCCCCCC(=O)CCCC(=O)C2CCC(c3cc(C(CC(=C)CC)CC4CCC(CCc5ccc6c(c5)C(C(=C)CCc5ccccc5)CCC6)C4)cc(C4C=C(C)C=C4C)c3)CC2)CCCCC(=O)CCCCCCCCCCC(=C)CCCC(=C)C2CCC(c3cc(C(CC(=C)CC)CC4CCC(CCc5ccc6c(c5)C(C(=C)CCc5ccccc5)CCC6)C4)cc(C4C=C(C)C=C4C)c3)CC2)C1]

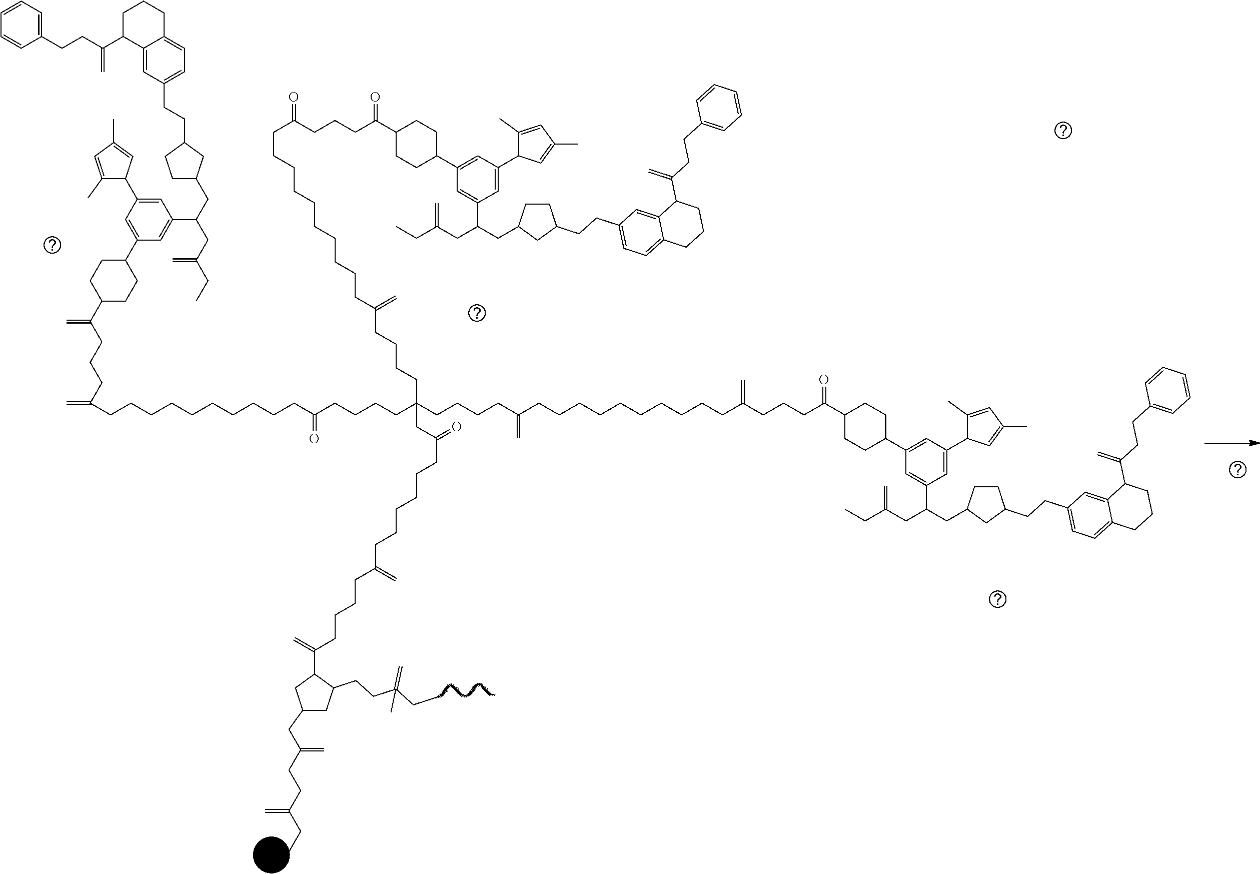

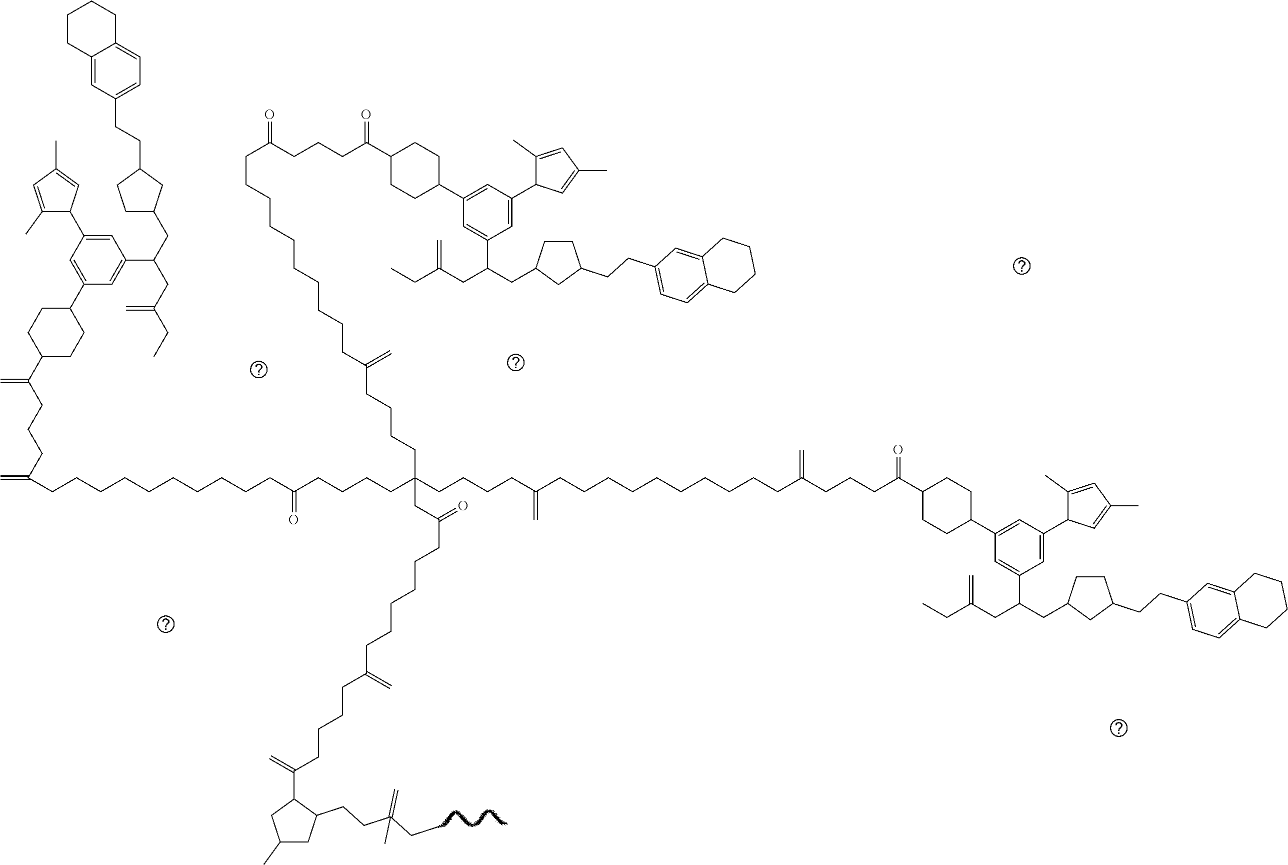

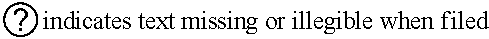

### Example 4: Evaluation of αvβ6 Integrin Targeting siRNA Conjugates

Table 2 depicts sense strand sequences and mass data for αvβ6 integrin targeting siRNA conjugates.

TABLE 2

| | | SEQ | | |
|---|---|---|---|---|
| | | ID | Theoretical | Found |
| Sense Strand | Sense Strand Sequence 5′ to 3′ | NO: | Mass | Mass |
| A-2783274 (121) | csasuuuuAfaUfCfCfucacucuaasasL333Z81 | 131 | 8873.904 | 8873.98 |
| A-2783272 (120) | csasuuuuAfaUfCfCfucacucuaasasL333Z80 | 132 | 11410.764 | 11410.82 |
| A-2686452 (132) | csasuuuuAfaUfCfCfucacucuaasasL352 | 133 | 7793.702 | 7792.61 |
| A-2686450 (135) | Q410scsauuuuAfaUfCfCfucacucuaasasL352 | 134 | 8724.808 | 8724.01 |
| A-2686451 (136) | Q411scsauuuuAfaUfCfCfucacucuaasasL353 | 135 | 8724.808 | 8724.02 |
| A-4881954 (511) | ususgggcAfaAfGfGfuggaaaugasasL373Z511 | 136 | 8341.215 | 8341.75 |
| A-4881955 (512) | ususgggcAfaAfGfGfuggaaaugasasL373Z512 | 137 | 8329.204 | 8329.88 |
| A-4881956 (513) | ususgggcAfaAfGfGfuggaaaugasasL373Z513 | 138 | 8369.269 | 8369.44 |
| A-3748001 (354) | ususgggcAfaAfGfGfuggaaaugasasL354 | 139 | 10056.464 | 10055.68 |

Table 3 depicts siRNA antisense (AS) and sense strand (SS) sequences of dsRNA agents targeting Sod1 mRNA used for in vitro and/or in vivo studies to evaluate pharmacodynamic and/or pharmacologic activity of αvβ6 integrin targeting siRNA conjugates.

TABLE 3

| | | | SEQ | |
|---|---|---|---|---|
| | | | ID | |
| Duplex ID | Strand ID | Sequence 5′ to 3′ | NO | MW (Da) |
| AD-1534460 | A-2783274 (SS) | csasuuuuAfaUfCfCfucacucuaasasL333Z81 | 131 | 8873.904 |
| | A-444402 (AS) | VPusUfsuagAfgUfGfaggaUfuAfaaaugsasg | 140 | 7851.156 |
| AD-1534458 | A-2783272 (SS) | csasuuuuAfaUfCfCfucacucuaasasL333Z80 | 132 | 11410.764 |
| | A-444402 (AS) | VPusUfsuagAfgUfGfaggaUfuAfaaaugsasg | 140 | 7851.156 |
| AD-1481903 | A-2686452 (SS) | csasuuuuAfaUfCfCfucacucuaasasL352 | 133 | 7793.702 |
| | A-444402 (AS) | VPusUfsuagAfgUfGfaggaUfuAfaaaugsasg | 140 | 7851.156 |
| AD-1481901 | A-2686450 (SS) | Q410scsauuuuAfaUfCfCfucacucuaasasL352 | 134 | 8724.808 |
| | A-444402 (AS) | VPusUfsuagAfgUfGfaggaUfuAfaaaugsasg | 140 | 7851.156 |
| AD-1481902 | A-2686451 (SS) | Q411scsauuuuAfaUfCfCfucacucuaasasL353 | 135 | 8724.808 |
| | A-444402 (AS) | VPusUfsuagAfgUfGfaggaUfuAfaaaugsasg | 140 | 7851.156 |
| AD-1427062 | A-2578434 (SS) | csasuuu(Uda)AfaUfCfCfucacucuasasa | 142 | 7128.132 |
| | A-444402 (AS) | VPusUfsuagAfgUfGfaggaUfuAfaaaugsasg | 140 | 7851.156 |
| AD-2639645 | A-4881954 (SS) | ususgggcAfaAfGfGfuggaaaugasasL373Z511 | 136 | 8341.75 |
| | A-859296 (AS) | VPusUfscauUfuCfCfaccuUfuGfcccaasgsu | 141 | 7532.01 |
| AD-2639646 | A-4881955 (SS) | ususgggcAfaAfGfGfuggaaaugasasL373Z512 | 137 | 8329.88 |
| | A-859296 (AS) | VPusUfscauUfuCfCfaccuUfuGfcccaasgsu | 141 | 7532.01 |
| AD-2639647 | A-4881956 (SS) | ususgggcAfaAfGfGfuggaaaugasasL373Z513 | 138 | 8369.44 |
| | A-859296 (AS) | VPusUfscauUfuCfCfaccuUfuGfcccaasgsu | 141 | 7532.01 |
| AD-2032892 | A-3748001 (SS) | ususgggcAfaAfGfGfuggaaaugasasL354 | 139 | 10055.68 |
| | A-859296 (AS) | VPusUfscauUfuCfCfaccuUfuGfcccaasgsu | 141 | 7532.01 |

### In Vivo Study—Mouse

Female C57BL/6 mice (8-10 weeks of age) (N=3) were dosed with a single intravenous bolus injection at 2 mg/kg diluted in 1×PBS. Terminal quadriceps, heart, lungs, and liver tissues were collected on day 21 after injection. Levels of Sod1 mRNA were quantified from powdered whole tissue by RT-qPCR. Target mRNA reduction was normalized to GAPDH mRNA for each sample and reported as relative reduction compared to control animals treated with PBS alone.

mRNA Quantification by RT-qPCR

Heart and quadricep powder were homogenized and lysed in 750 μL of QIAzol Lysis Reagent for 5 minutes×2 at 25 Hz using a TissueLyser. Chloroform (150 L) was thoroughly mixed with each sample and lysates were centrifugated at full speed for 15 minutes at 4° C. Total RNA was isolated from the supernatant using the RNeasy® 96 Universal Tissue Kit. Resulting RNA (1500 ng) was used for cDNA synthesis using the Applied Biosystems™ High-Capacity cDNA Reverse Transcription Kit with RNase Inhibitor. For qPCR reactions, 2 uL cDNA was used per 10 μL reaction. RT-qPCR was performed using the LightCycler® 480 Probes Master in the LightCycler® 480 Instrument system using gene specific TaqMan assays for each target mouse-specific Sod1 (ThermoFisher Scientific, Mm01344233_g1) and Gapdh (ThermoFisher Scientific, Mm99999915_g1) were used for RT-qPCR analysis. Each sample was analyzed in duplicate by RT-qPCR. Data were analyzed using the ΔΔCt method normalizing to control animals treated with 1×PBS alone.

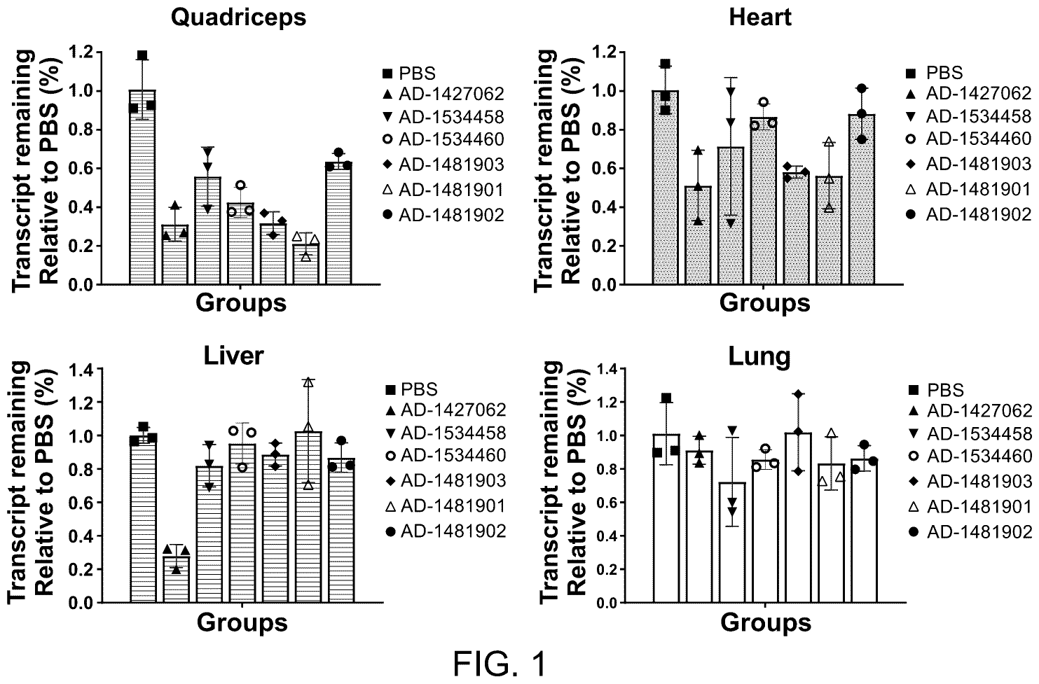

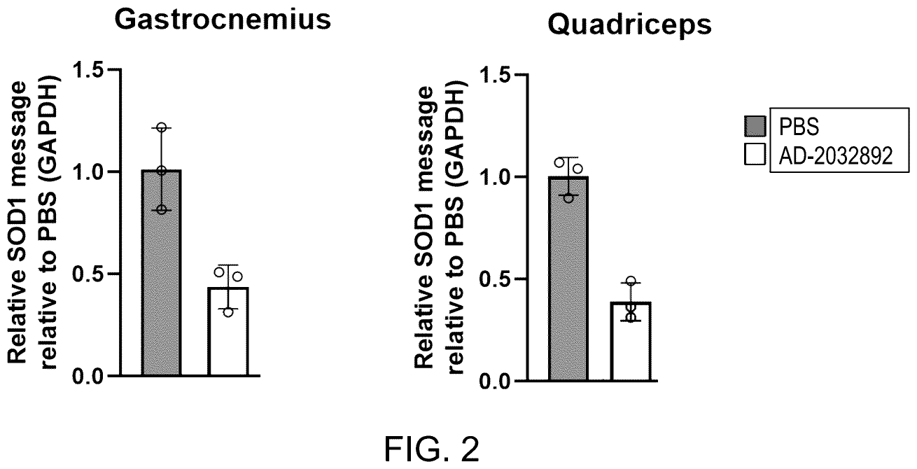

As depicted in FIGS. 1 and 2, intravenous administration of a single dose of the agents preferentially and potently inhibits expression of Sod1 in mouse quadriceps tissue.

### In Vivo Study—Cynomolgus Monkey

Female cynomolgus monkeys (N=3) were dosed with a single intravenous injection in the left saphenous vein at 10 mg/kg diluted in 1×PBS. Terminal skeletal tissues indicated were collected 28 days after injection. Tissues were homogenized by TissueLyser plus ball bearing (50 cycles/sec for 2 mins) in a guanidine-based lysis buffer. RNA was extracted using Qiagen's RNeasy Fibrous Tissue Mini Kit Catalog #7470, using manual columns and including a proteinase K digestion at 55° C. for 10 minutes. Levels of Sod1 mRNA were quantified from powdered whole tissue by RT-qPCR, using Invitrogen SuperScript IV VILO Master Mix, Applied Biosystems ViiA7 RT-PCR system, and Applied Biosystems TaqMan Fast Advanced Master Mix. Target mRNA reduction was normalized to PPIB mRNA for each sample and reported as relative reduction (ΔΔCT method) compared to control animals treated with PBS alone.

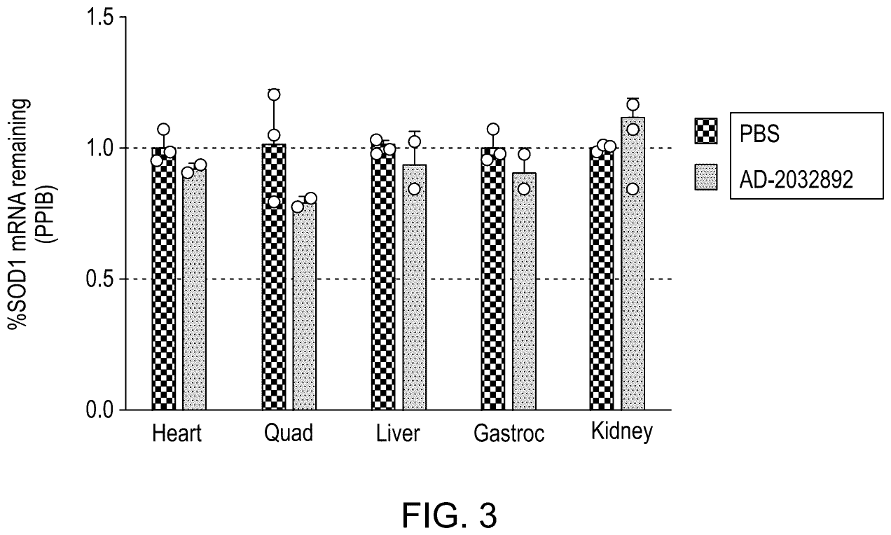

As depicted in FIG. 3, intravenous administration of a single dose of the agents potently inhibits expression of Sod1 in skeletal muscle tissue.

### In Vivo Study—Mouse

C57Bl/6 female mice were dosed with a single intratracheal administration of αvβ6 targeting siRNA conjugates at either 0.5 mg/kg diluted in 1×PBS or vehicle control. Terminal lung tissues were then collected 21 days after test article administration. Frozen lung tissue was powdered using liquid nitrogen in the GenoGrinder at 1250 rpm for 90 seconds. Powdered tissues were then added to Metal Bead Lysing Matrix (MP Bio, REF 6925100, lot 169952). In a fume hood, 1 mL of Qiazol (Qiagen 79306, lot #57201569) was added to each tube followed by further homogenization in the TissueLyser II (shaking at 25 Hz for 2 minutes, repeating until tissue was dissolved). Bead tubes were spun at 12,000×g for 5 minutes and Qiazol layer transferred into new tubes. In a fume hood, 200 μL of chloroform were added (Sigma Aldrich 288306) to each tube, followed by vigorous shaking for 15 seconds. Samples were centrifuged at 12,000×g for 15 minutes at 4° C. and aqueous phase then transferred to new tube. RNA was then extracted using QIAcube Connect according to manufacturer's protocol for RNeasy plus universal. Levels of mouse Sod1 mRNA (using ThermoFisher probe Mm01344233_g1) were quantified from powdered whole tissue by RT-qPCR, using Invitrogen SuperScript IV VILO Master Mix (#11756500), ThermoFisher Quantstudio 5 RT-PCR system, and TaqMan Fast Advanced Master Mix (Applied Biosystems, 4444554). Target mRNA reduction was normalized to UBC mRNA (Thermofisher probe Mm01201237 ml) for each sample and reported as relative reduction (ΔΔCT method) compared to control animals treated with PBS alone.

### In Vivo Study—Mouse

C57Bl/6 female mice were dosed with a single intranasal administration of αvβ6 targeting siRNA conjugates at either 3 or 10 mg/kg diluted in 1×PBS or vehicle control. Terminal lung tissues were then collected 21 days after test article administration. Frozen lung tissue was powdered using liquid nitrogen in the GenoGrinder at 1250 rpm for 90 seconds. Powdered tissues were then added to Metal Bead Lysing Matrix (MP Bio, REF 6925100, lot 169952,). In a fume hood, 1 mL of Qiazol (Qiagen 79306, lot #57201569) was added to each tube followed by further homogenization in the TissueLyser II (shaking at 25 Hz for 2 minutes, repeating until tissue was dissolved). Bead tubes were spun at 12,000×g for 5 minutes and Qiazol layer transferred into new tubes. In a fume hood, 200 μL of chloroform were added (Sigma Aldrich 288306) to each tube, followed by vigorous shaking for 15 seconds. Samples were centrifuged at 12,000×g for 15 minutes at 4° C. and aqueous phase then transferred to new tube. RNA was then extracted using QIAcube Connect according to manufacturer's protocol for RNeasy plus universal. Levels of mouse Sod1 mRNA (using ThermoFisher probe Mm01344233_g1) were quantified from powdered whole tissue by RT-qPCR, using Invitrogen SuperScript IV VILO Master Mix (#11756500), ThermoFisher Quantstudio 5 RT-PCR system, and TaqMan Fast Advanced Master Mix (Applied Biosystems, 4444554). Target mRNA reduction was normalized to UBC mRNA (Thermofisher probe Mm01201237 ml) for each sample and reported as relative reduction (ΔΔCT method) compared to control animals treated with PBS alone.

### In Vivo Study—Mouse

C57Bl/6 female mice were dosed with a single oropharyngeal aspiration administration of αvβ6 targeting siRNA conjugates at either 2 mg/kg diluted in 1×PBS or vehicle control. Terminal lung tissues were then collected 21 days after test article administration. Frozen lung tissue was powdered using liquid nitrogen in the GenoGrinder at 1250 rpm for 90 seconds. Powdered tissues were then added to Metal Bead Lysing Matrix (MP Bio, REF 6925100, lot 169952). In a fume hood, 1 mL of Qiazol (Qiagen 79306, lot #57201569) was added to each tube followed by further homogenization in the TissueLyser II (shaking at 25 Hz for 2 minutes, repeating until tissue was dissolved). Bead tubes were spun at 12,000×g for 5 minutes and Qiazol layer transferred into new tubes. In a fume hood, 200 μL of chloroform were added (Sigma Aldrich 288306) to each tube, followed by vigorous shaking for 15 seconds. Samples were centrifuged at 12,000×g for 15 minutes at 4° C. and aqueous phase then transferred to new tube. RNA was then extracted using QIAcube Connect according to manufacturer's protocol for RNeasy plus universal. Levels of mouse Sod1 mRNA (using ThermoFisher probe Mm01344233_g1) were quantified from powdered whole tissue by RT-qPCR, using Invitrogen SuperScript IV VILO Master Mix (#11756500), ThermoFisher Quantstudio 5 RT-PCR system, and TaqMan Fast Advanced Master Mix (Applied Biosystems, 4444554). Target mRNA reduction was normalized to UBC mRNA (Thermofisher probe Mm01201237_ml) for each sample and reported as relative reduction (ΔΔCT method) compared to control animals treated with PBS alone.

In Vivo Study—Macaca fascicularis

Female Macaca fascicularis were dosed with a single intravenous administration of αvβ6 targeting siRNA conjugates at either 5 or 10 mg/kg diluted in 1×PBS or vehicle control. Terminal lung tissues were then collected 30 days after test article administration. Frozen lung tissue was powdered using liquid nitrogen in the GenoGrinder at 1250 rpm for 90 seconds. Powdered tissues were then added to Metal Bead Lysing Matrix (MP Bio, REF 6925100, lot 169952). In a fume hood, 1 mL of Qiazol (Qiagen 79306, lot #57201569) was added to each tube followed by further homogenization in the TissueLyser II (shaking at 25 Hz for 2 minutes, repeating until tissue was dissolved). Bead tubes were spun at 12,000×g for 5 minutes and Qiazol layer transferred into new tubes. In a fume hood, 200 μL of chloroform were added (Sigma Aldrich 288306) to each tube, followed by vigorous shaking for 15 seconds. Samples were centrifuged at 12,000×g for 15 minutes at 4° C. and aqueous phase then transferred to new tube. RNA was then extracted using QIAcube Connect according to manufacturer's protocol for RNeasy plus universal. Levels of Macaca fascicularis Sod1 mRNA (using ThermoFisher probe Mf04363557 ml) were quantified from powdered whole tissue by RT-qPCR, using Invitrogen SuperScript IV VILO Master Mix (#11756500), ThermoFisher Quantstudio 5 RT-PCR system, and TaqMan Fast Advanced Master Mix (Applied Biosystems, 4444554). Target mRNA reduction was normalized to UBC mRNA (Thermofisher probe Mf02798368_ml) for each sample and reported as relative reduction (ΔΔCT method) compared to control animals treated with PBS alone.

### In Vitro Study—ELISA Displacement Assay

ELISA plates were coated with 0.6 ug/mL recombinant human LAP/TGF1b (R&D Systems 246LP025) in carbonate buffer (pH 9.6) overnight at 4 C. Plates were washed with PBST and blocked for 1 hour at room temperature with assay buffer (20 mM Tris-HCl, 150 mM NaCl, 2 mM CaCl₂, 2 mM MgCl₂, 2 mM MnCl₂, 0.5% BSA, pH 7.4). Serial dilutions of competitor compound in assay buffer were added to plate, as well as 1.0 ug/mL of purified recombinant human αvβ6 (R&D Systems 3817AV050), and samples were incubated for 1 hour at room temperature. Plates were washed with PBS-T, then incubated with 2.5 ug/mL of anti-integrin av primary antibody (Sigma MAB1978) for 1 hour at room temperature. Plates were washed with PBS-T, then incubated with 2.0 ug/mL of HRP-conjugated secondary antibody for 1 hour at room temperature. Samples were incubated with TMB substrate (Thermo 34024) for 15 minutes, reaction was quenched with 3 M sulfuric acid, and absorbance was measured at 450 nm. Percent binding inhibition was calculated by normalizing to a maximum value obtained from no-competitor samples and a minimum value obtained from no-competitor-no-integrin samples. EC50 values are reported in the following ranges: “A” EC50>100 nM; “B” EC50 10-100 nM; “C” EC50<10 nM.

TABLE 4
αvβ6 integrin binding efficiencies of duplexes
as measured by ELISA displacement assay.

| Duplex | αvβ6 EC₅₀ |
|---|---|
| AD-2032892 | C |
| AD-2639645 | A |
| AD-2639646 | B |
| AD-2639647 | C |

### Example 5: Evaluation of αvβ6 Integrin Conjugates in Mouse Lung

αvβ6 is highly restricted to a subset of epithelial cells including the epithelium lining the conducting airways and alveoli and is constitutively expressed at low levels in uninjured epithelia. Expression is markedly upregulated on epithelial cells in multiple epithelial organs, including the lung, in response to injury and acute and chronic inflammation. In addition, αvβ6 integrin is constitutively bound to TGF-α, suggesting that the system is primed to detect injurious stimuli and integrins play a role in cell migration and as such, a significant role in cancer metastasis.

Accordingly, to evaluate the effect of αvβ6 integrin conjugates in vivo, a structure-activity relationship (SAR) analysis of dsRNA agents targeting Sod1 mRNA conjugated to αvβ6 was conducted

Table 5 provides the study design used to evaluate the in vivo pharmacodynamic and/or pharmacologic activity of αvβ6 integrin targeting siRNA conjugates in mouse lung following intranasal administration.

Table 6 provides the unmodified nucleotide sequences of the antisense and sense strands of dsRNA agents targeting Sod1 mRNA used in the study and Table 7 provides the modified nucleotide sequences of the antisense and sense strands of dsRNA agents targeting Sod1 mRNA used in the study.

TABLE 5
Study Design

| | | | Route of | |
|---|---|---|---|---|
| | | | Administration | Sacrifice |
| Group | Test Article ID | Dose (mg/kg) | and Dose Day | Day |
| 1 | PBS | — | Intranasal | Day 10 |
| 2 control | AD-401824 | 1 | (50 μL total) | |
| | (+C16) | | on Day 0 | |
| 3 control | AD-463791 | | | |
| | (unconjugated) | | | |
| 4 | AD-1481899 | | | |
| 5 | AD-1481900 | | | |
| 6 | AD-1481901 | | | |
| 7 | AD-1481902 | | | |
| 8 | AD-1481903 | | | |
| 9 | AD-1481904 | | | |
| 10 control | AD-401824 | 10 | | |
| | (+C16) | | | |
| 11 control | AD-463791 | | | |
| | (unconjugated) | | | |
| 12 | AD-1481899 | | | |
| 13 | AD-1481900 | | | |
| 14 | AD-1481901 | | | |
| 15 | AD-1481902 | | | |
| 16 | AD-1481903 | | | |
| 17 | AD-1481904 | | | |

TABLE 6
Unmodified Sense and Antisense Strand Sequences of Sod1 dsRNA Agents

| | | SEQ | | | SEQ | |
|---|---|---|---|---|---|---|
| | Sense | ID | | Antisense | ID | |
| Duplex Name | Sequence 5′ to 3′ | No: | Range* | Sequence 5′ to 3′ | No: | Range* |
| AD-1481899 | CAUUUUAAUCCUCACUCUAAA | 143 | 255-275 | UUUAGAGUGAGGAUUAAAAUGAG | 144 | 253-275 |
| AD-1481900 | CAUUUUAAUCCUCACUCUAAA | 143 | 255-275 | UUUAGAGUGAGGAUUAAAAUGAG | 144 | 253-275 |
| AD-1481901 | CAUUUUAAUCCUCACUCUAAA | 143 | 255-275 | UUUAGAGUGAGGAUUAAAAUGAG | 144 | 253-275 |
| AD-1481902 | CAUUUUAAUCCUCACUCUAAA | 143 | 255-275 | UUUAGAGUGAGGAUUAAAAUGAG | 144 | 253-275 |
| AD-1481903 | CAUUUUAAUCCUCACUCUAAA | 143 | 255-275 | UUUAGAGUGAGGAUUAAAAUGAG | 144 | 253-275 |
| AD-1481904 | CAUUUUAAUCCUCACUCUAAA | 143 | 255-275 | UUUAGAGUGAGGAUUAAAAUGAG | 144 | 253-275 |
| AD-401824 | CAUUUUAAUCCUCACUCUAAA | 143 | 255-275 | UUUAGAGUGAGGAUUAAAAUGAG | 144 | 253-275 |
| AD-463791 | CAUUUUAAUCCUCACUCUAAA | 143 | 255-275 | UUUAGAGUGAGGAUUAAAAUGAG | 144 | 253-275 |

*Range in NM_000454.5

TABLE 7
Modified Sense and Antisense Strand Sequences of Sod1 dsRNA Agents

| | | SEQ | | SEQ | | SEQ |
|---|---|---|---|---|---|---|
| Duplex | Sense | ID | Antisense | ID | mRNA | ID |
| Name | Sequence 5′ to 3′ | No: | Sequence 5′ to 3′ | No: | target sequence | No: |
| AD-1481899 | csasuuuuAfaUfCfCfucac | 145 | VPusUfsuagAfgUfGfaggaUf | 153 | CUCAUUUUAAUCCUCAC | 154 |
| | ucuaasasL354 | | uAfaaaugsasg | | UCUAAG | |
| AD-1481900 | csasuuuuAfaUfCfCfucac | 146 | VPusUfsuagAfgUfGfaggaUf | 153 | CUCAUUUUAAUCCUCAC | 154 |
| | ucuaasasL355 | | uAfaaaugsasg | | UCUAAG | |
| AD-1481901 | Q410scsauuuuAfaUfCfCf | 147 | VPusUfsuagAfgUfGfaggaUf | 153 | CUCAUUUUAAUCCUCAC | 154 |
| | ucacucuaasasL352 | | uAfaaaugsasg | | UCUAAG | |
| AD-1481902 | Q411scsauuuuAfaUfCfCf | 148 | VPusUfsuagAfgUfGfaggaUf | 153 | CUCAUUUUAAUCCUCAC | 154 |
| | ucacucuaasasL353 | | uAfaaaugsasg | | UCUAAG | |
| AD-1481903 | csasuuuuAfaUfCfCfucac | 149 | VPusUfsuagAfgUfGfaggaUf | 153 | CUCAUUUUAAUCCUCAC | 154 |
| | ucuaasasL352 | | uAfaaaugsasg | | UCUAAG | |
| AD-1481904 | csasuuuuAfaUfCfCfucac | 150 | VPusUfsuagAfgUfGfaggaUf | 153 | CUCAUUUUAAUCCUCAC | 154 |
| | ucuaasasL353 | | uAfaaaugsasg | | UCUAAG | |
| AD-401824 | csasuuu(Uhd)AfaUfCfCf | 151 | VPusUfsuagAfgUfGfaggaUf | 153 | CUCAUUUUAAUCCUCAC | 154 |
| | ucacucuasasa | | uAfaaaugsasg | | UCUAAG | |
| AD-463791 | csasuuuuAfaUfCfCfucac | 152 | VPusUfsuagAfgUfGfaggaUf | 153 | CUCAUUUUAAUCCUCAC | 154 |
| | ucuasasa | | uAfaaaugsasg | | UCUAAG | |

| Duplex ID | Ligand Stereochemistry | Avidity |
|---|---|---|
| AD-1481899.1 | (R,S) | Triple |
| AD-1481900.1 | (R,R) | Triple |
| AD-1481901.1 | (R,S) | Bis 3′,5′ |
| AD-1481902.1 | (R,R) | Bis 3′,5′ |
| AD-1481903.1 | (R,S) | Mono 3′ |
| AD-1481904.1 | (R,R) | Mono 3′ |

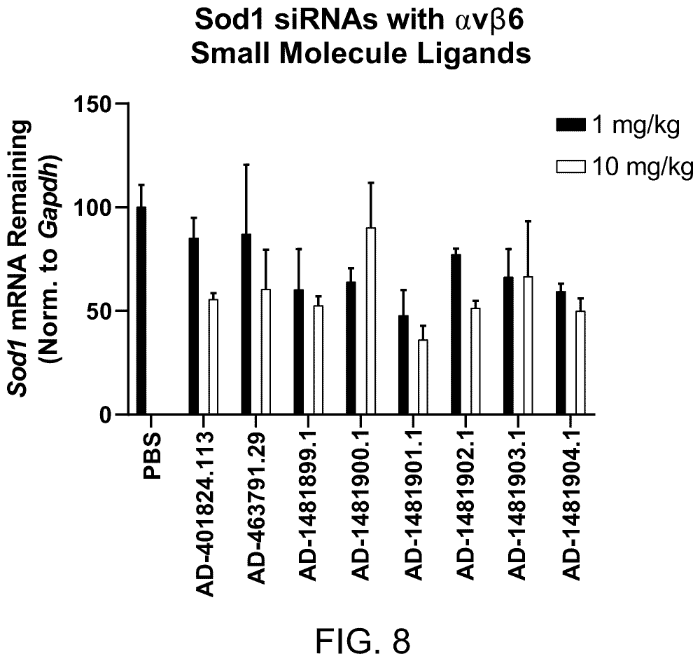

Female mice were intranasally administered either a single 1 mg/kg or 10 mg/kg dose of the dsRNA agents conjugated to αvβ6 and at Day 10 post-dose animals were sacrificed and lung, heart, liver, and kidney tissues were collected. The level of Sod1 mRNA in the samples was determined by R^{T}-qPCR as described above. The knockdown of Sod1 mRNA in lung tissue by the αvβ6-Sod1 dsRNA agents is shown in Table 8 and FIG. 8.

TABLE 8
αvβ6 Sod1 siRNA Knockdown in Mouse Lung

| | | % Sod1 | |
|---|---|---|---|
| Dose | Test Article | Remaining | Stdev |
| | PBS | 100.0 | 10.8 |
| 1 mg/kg | AD-401824.113 | 85.1 | 9.8 |
| | AD-463791.29 | 86.9 | 33.6 |
| | AD-1481899.1 | 60.0 | 19.8 |
| | AD-1481900.1 | 63.8 | 6.7 |
| | AD-1481901.1 | 47.7 | 12.4 |
| | AD-1481902.1 | 77.2 | 2.9 |
| | AD-1481903.1 | 66.2 | 13.6 |
| | AD-1481904.1 | 59.2 | 4.0 |
| 10 mg/kg | AD-401824.113 | 55.4 | 3.2 |
| | AD-463791.29 | 60.3 | 19.2 |
| | AD-1481899.1 | 52.4 | 4.6 |
| | AD-1481900.1 | 90.1 | 21.7 |
| | AD-1481901.1 | 35.9 | 6.9 |
| | AD-1481902.1 | 51.2 | 3.6 |
| | AD-1481903.1 | 66.5 | 26.8 |
| | AD-1481904.1 | 49.8 | 6.3 |

The data demonstrate that there is a preference for (S,R) ligands over (S,S) in all conjugates except for 3′-monos (AD-1481903 (S,R) vs AD-1481904 (S,S)). In addition, the correct ligand stereochemistry improves binding by 2-3 logs. Thus, there is less activity with (S,S) ligand, not no activity. Furthermore, 3′,5′ is better than the triple (AD-1481899 vs AD-1481901 as matched pair of (S,R) stereochemistry).

TABLE 1
Abbreviations of nucleotide monomers used in nucleic acid sequence representation. It will be
understood that these monomers, when present in an oligonucleotide, are mutually linked by
3′ → 5′ phosphodiester bonds; and it is understood that when the nucleotide contains a 2′-fluoro modification,
then the fluoro replaces the hydroxy at that position in the parent nucleotide (i.e., it is a 2′-deoxy-2′-
fluoronucleotide). The abbreviations are understood to omit the 3′-phosphate (i.e. they are 3′-OH) when
placed at the 3′-terminal position of an oligonucleotide (unless the 3′-end is linked to a ligand).

| Abbv. | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| Y34 | 2-O-methyl-1-deoxyribofuranose 3-phosphate (abasic 2′-OMe furanose) |
| | 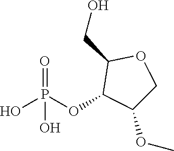 |
| Y44 | inverted abasic DNA (1,2-dideoxyribofuranose 5-phosphate) |
| | 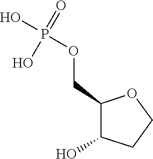 [SMILES: O=P(O)(O)OC[C@H]1OCC[C@@H]1O] |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3-phosphate |
| dCs | 2′-deoxycytidine-3-phosphorothioate |
| dG | 2′-deoxyguanosine-3-phosphate |
| dGs | 2′-deoxyguanosine-3′-phosphorothioate |
| dT | 2′-deoxythimidine-3′-phosphate |
| dTs | 2′-deoxythimidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Ahd) | 2′-O-hexadecyl-adenosine-3′-phosphate |
| (Ahds) | 2′-O-hexadecyl-adenosine-3′-phosphorothioate |
| (Chd) | 2′-O-hexadecyl-cytidine-3′-phosphate |
| | 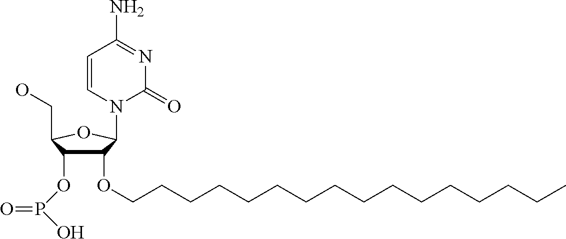 |
| (Chds) | 2′-O-hexadecyl-cytidine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Uhd) | 2′-O-hexadecyl-uridine-3′-phosphate |
| (Uhds) | 2′-O-hexadecyl-uridine-3′-phosphorothioate |
| (Ada) | 2′-O-docosyladenosine 3′-phosphate |
| | 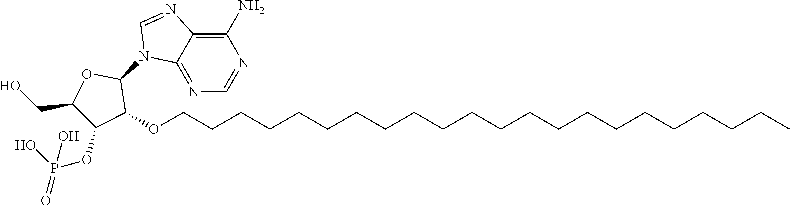 [SMILES: CCCCCCCCCCCCCCCCCCCCCCO[C@@H]1[C@H](OP(=O)(O)O)[C@@H](CO)O[C@H]1n1cnc2c(N)ncnc21] |
| (Cda) | 2′-O-docosylcytidine 3′-phosphate |
| | 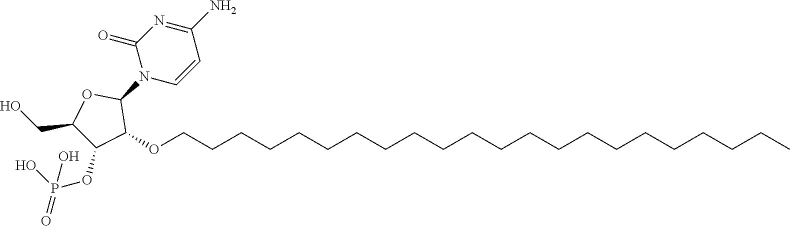 [SMILES: CCCCCCCCCCCCCCCCCCCCCCO[C@@H]1[C@H](OP(=O)(O)O)[C@@H](CO)O[C@H]1n1ccc(N)nc1=O] |
| (Gda) | 2′-O-docosylguanosine 3′-phosphate |
| | 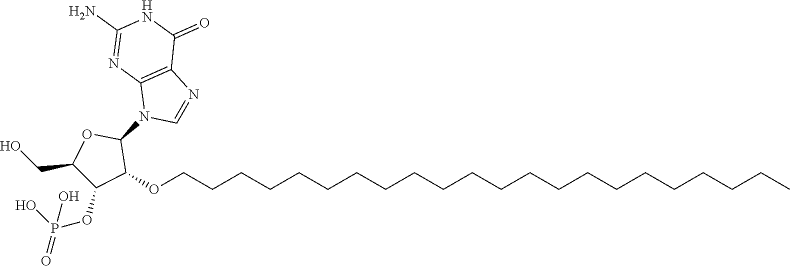 [SMILES: CCCCCCCCCCCCCCCCCCCCCCO[C@@H]1[C@H](OP(=O)(O)O)[C@@H](CO)O[C@H]1n1cnc2c(=O)[nH]c(N)nc21] |
| (Uda) | 2′-O-docosyluridine 3′-phosphate |
| | 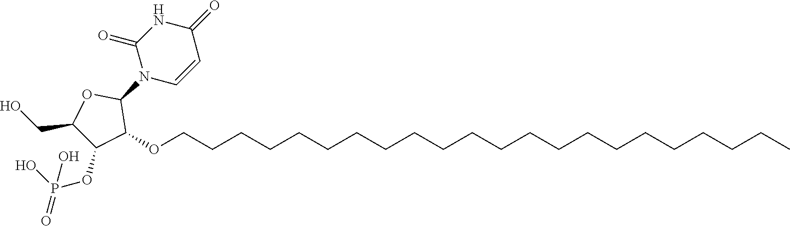 [SMILES: CCCCCCCCCCCCCCCCCCCCCCO[C@@H]1[C@H](OP(=O)(O)O)[C@@H](CO)O[C@H]1n1ccc(=O)[nH]c1=O] |
| L333 | 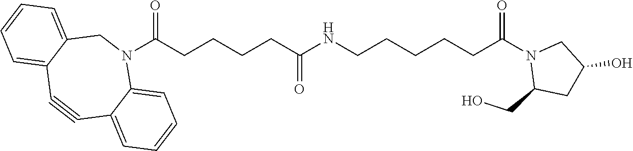 [SMILES: O=C(CCCCC(=O)N1Cc2ccccc2C#Cc2ccccc21)NCCCCCC(=O)N1C[C@H](O)C[C@H]1CO] |
| | 1-(6-(6-(dibenzo[b,f]azacyclooct-4-yn-5-yl)-6-oxohexan-1-oylamino)hexan-1-oyl)-4R- |
| | hydroxy-2S-hydroxymethylpyrrolidine |
| L333 Z### | 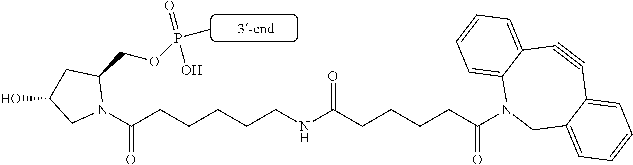 [SMILES: CP(=O)(O)OC[C@@H]1C[C@@H](O)CN1C(=O)CCCCCNC(=O)CCCCC(=O)N1Cc2ccccc2C#Cc2ccccc21] |
| | (1-(6-(6-(dibenzo[b,f]azacyclooct-4-yn-5-yl)-6-oxohexan-1-oylamino)hexan-1-oyl)-4R- |
| | hydroxypyrrolidin-2S-yl)methyl ester, click conjugate triazole formed between the |
| | dibenzoazacyclooctyne group and the azide group of Z### |
| L333Z80 | 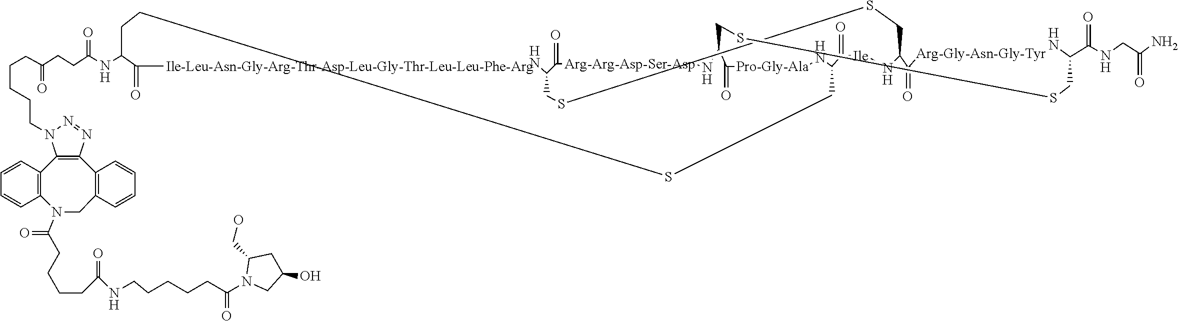 |
| | 1-(6-(6-(dibenzo[b,f]azacyclooct-4-yn-5-yl)-6-oxohexan-1-oylamino)hexan-1-oyl)-4R- |
| | hydroxy-2S-hydroxymethylpyrrolidine, click conjugate triazole formed between the |
| | dibenzoazacyclooctyne group and the azide group of Z80 (SEQ ID NO: 155) |
| L333Z81 | 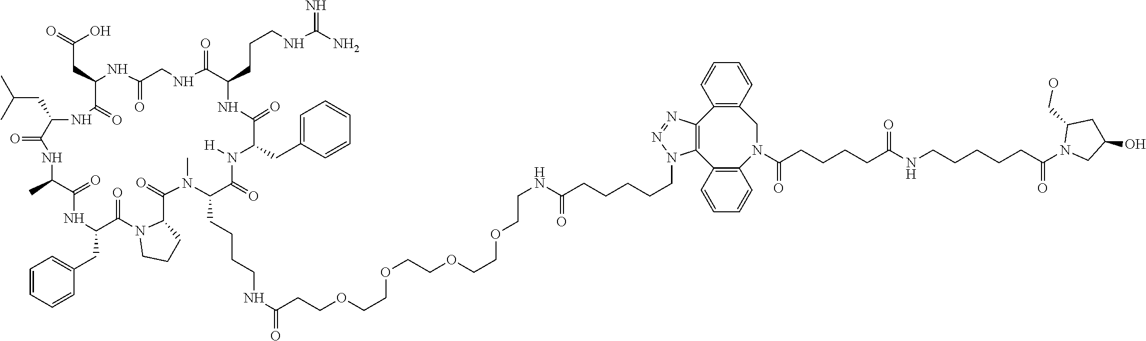 [SMILES: CC(C)C[C@@H]1NC(=O)[C@@H](CC(=O)O)NC(=O)CNC(=O)[C@@H](CCCNC(=N)N)NC(=O)[C@H](Cc2ccccc2)NC(=O)[C@H](CCCCNC(=O)CCOCCOCCOCCOCCNC(=O)CCCCCn2nnc3c2-c2ccccc2N(C(=O)CCCCC(=O)NCCCCCC(=O)N2C[C@H](O)C[C@H]2CO)Cc2ccccc2-3)N(C)C(=O)[C@@H]2CCCN2C(=O)[C@H](Cc2ccccc2)NC(=O)[C@@H](C)NC1=O] |
| | 1-(6-(6-(dibenzo[b,f]azacyclooct-4-yn-5-yl)-6-oxohexan-1-oylamino)hexan-1-oyl)-4R- |
| | hydroxy-2S-hydroxymethylpyrrolidine, click conjugate triazole formed between the |
| | dibenzoazacyclooctyne group and the azide group of Z81 (SEQ ID NO: 156) |
| L352 | 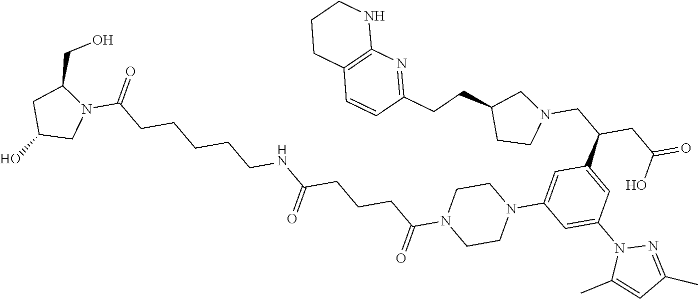 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCCCC(=O)N4C[C@H](O)C[C@H]4CO)CC3)c2)n1] |
| | (S)-3-(3-(3,5-dimethyl-1H-pyrazol-1-yl)-5-(4-(5-((6-((2S,4R)-4-hydroxy-2- |
| | (hydroxymethyl)pyrrolidin-1-yl)-6-oxohexyl)amino)-5-oxopentanoyl)piperazin-1- |
| | yl)phenyl)-4-((R)-3-(2-(5,6,7,8-tetrahydro-1,8-naphthyridin-2-yl)ethyl)pyrrolidin-1- |
| | yl)butanoic acid |
| L353 | 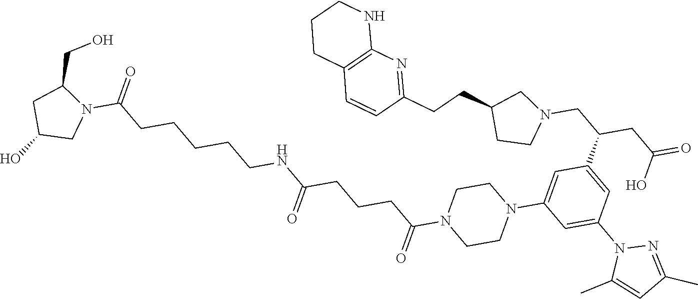 [SMILES: Cc1cc(C)n(-c2cc([C@@H](CC(=O)O)CN3CC[C@@H](CCc4ccc5c(n4)NCCC5)C3)cc(N3CCN(C(=O)CCCC(=O)NCCCCCC(=O)N4C[C@H](O)C[C@H]4CO)CC3)c2)n1] |
| | (R)-3-(3-(3,5-dimethyl-1H-pyrazol-1-yl)-5-(4-(5-((6-((2S,4R)-4-hydroxy-2- |
| | (hydroxymethyl)pyrrolidin-1-yl)-6-oxohexyl)amino)-5-oxopentanoyl)piperazin-1- |
| | yl)phenyl)-4-((R)-3-(2-(5,6,7,8-tetrahydro-1,8-naphthyridin-2-yl)ethyl)pyrrolidin-1- |
| | yl)butanoic acid |
| L354 | 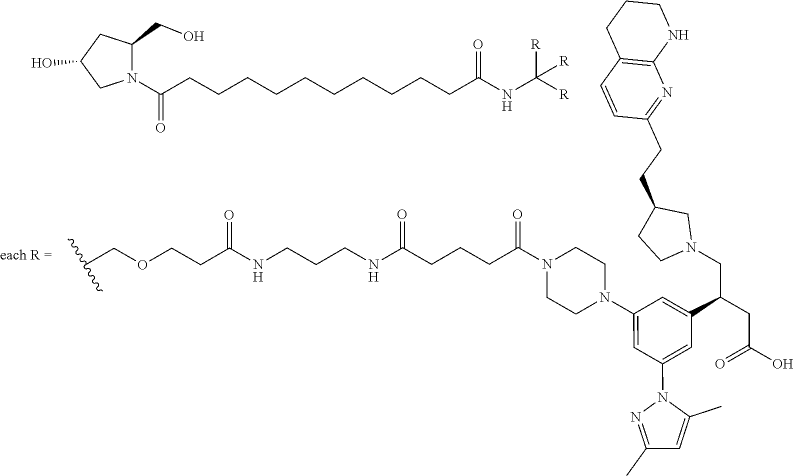 |
| | (2S,4R)-1-[23-((5-(4-(3-((S)-1-carboxy-3-((R)-3-(2-(5,6,7,8-tetrahydro-1,8-naphthyridin-2- |
| | yl)ethyl)pyrrolidin-1-yl)propan-2-yl)-5-(3,5-dimethyl-1H-pyrazol-1-yl)phenyl)piperazin-1- |
| | yl)-5-oxopentan-1-oyl)amino)-14,14-bis[3-(3-((5-(4-(3-((S)-1-carboxy-3-((R)-3-(2- |
| | (5,6,7,8-tetrahydro-1,8-naphthyridin-2-yl)ethyl)pyrrolidin-1-yl)propan-2-yl)-5-(3,5- |
| | dimethyl-1H-pyrazol-1-yl)phenyl)piperazin-1-yl)-5-oxopentan-1-oyl)amino)propylamino)- |
| | 3-oxo-propoxymethyl]-1,12,19-trioxo-16-oxa-13,20-diazatricos-1-yl]-4-hydroxy-2- |
| | hydroxymethylpyrrolidine |
| L355 | 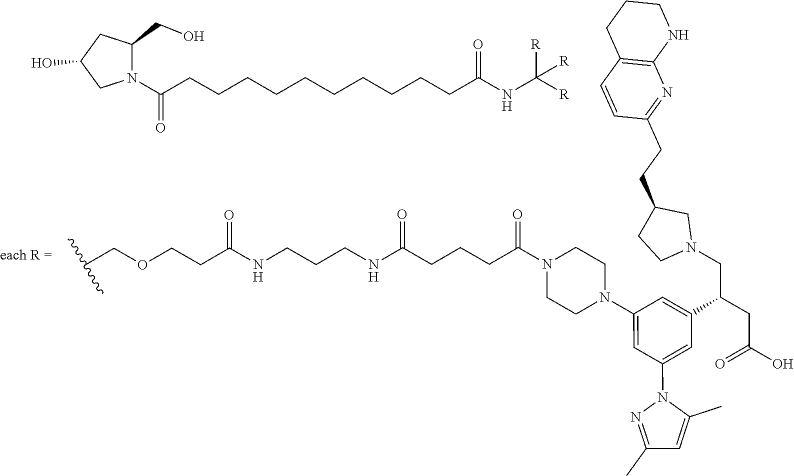 |
| | (2S,4R)-1-[23-((5-(4-(3-((R)-1-carboxy-3-((R)-3-(2-(5,6,7,8-tetrahydro-1,8-naphthyridin- |
| | 2-yl)ethyl)pyrrolidin-1-yl)propan-2-yl)-5-(3,5-dimethyl-1H-pyrazol-1-yl)phenyl)piperazin- |
| | 1-yl)-5-oxopentan-1-oyl)amino)-14,14-bis[3-(3-((5-(4-(3-((R)-1-carboxy-3-((R)-3-(2- |
| | (5,6,7,8-tetrahydro-1,8-naphthyridin-2-yl)ethyl)pyrrolidin-1-yl)propan-2-yl)-5-(3,5- |
| | dimethyl-1H-pyrazol-1-yl)phenyl)piperazin-1-yl)-5-oxopentan-1-oyl)amino)propylamino)- |
| | 3-oxo-propoxymethyl]-1,12,19-trioxo-16-oxa-13,20-diazatricos-1-yl]-4-hydroxy-2- |
| | hydroxymethylpyrrolidine |
| L373 Z### | 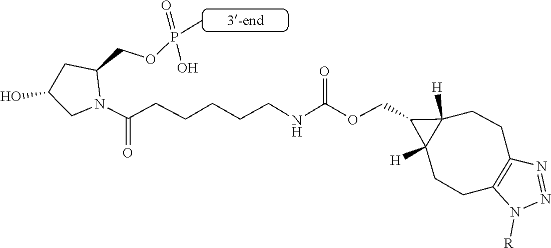 |
| | ((2S,4R)-(1-(6-(((1R,8S,9S)-bicyclo[6.1.0]non-4-yn-9-ylmethoxy)carbonylamino)hexan-1- |
| | oyl)-4-hydroxypyrrolidin-2-yl)methyl)ester, click conjugate triazole formed between the |
| | cyclooctyne group and the azide group of Z### |
| L373Z511 | 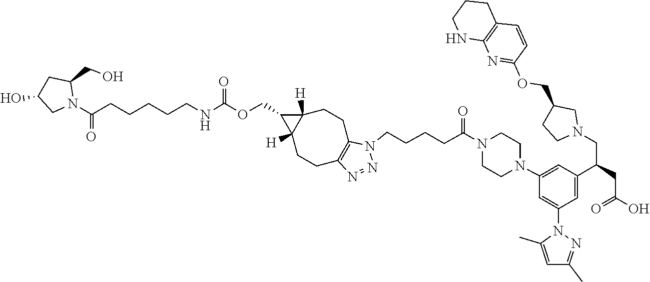 [SMILES: Cc1cc(C)n(-c2cc([C@H](CC(=O)O)CN3CC[C@@H](COc4ccc5c(n4)NCCC5)C3)cc(N3CCN(C(=O)CCCCn4nnc5c4CC[C@@H]4[C@H](CC5)[C@@H]4COC(=O)NCCCCCC(=O)N4C[C@H](O)C[C@H]4CO)CC3)c2)n1] |
| | ((2S,4R)-(1-(6-(((1R,8S,9S)-bicyclo[6.1.0]non-4-yn-9-ylmethoxy)carbonylamino)hexan-1- |
| | oyl)-4-hydroxypyrrolidin-2-yl)methyl)ester, click conjugate triazole formed between the |
| | cyclooctyne group and the azide group of Z511 |
| L373Z512 | 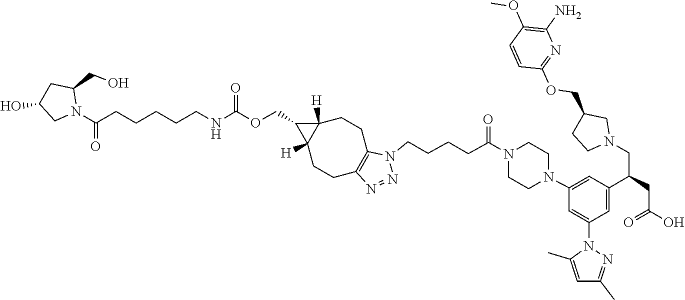 [SMILES: COc1ccc(OC[C@@H]2CCN(C[C@@H](CC(=O)O)c3cc(N4CCN(C(=O)CCCCn5nnc6c5CC[C@@H]5[C@H](CC6)[C@@H]5COC(=O)NCCCCCC(=O)N5C[C@H](O)C[C@H]5CO)CC4)cc(-n4nc(C)cc4C)c3)C2)nc1N] |
| | ((2S,4R)-(1-(6-(((1R,8S,9S)-bicyclo[6.1.0]non-4-yn-9-ylmethoxy)carbonylamino)hexan-1- |
| | oyl)-4-hydroxypyrrolidin-2-yl)methyl)ester, click conjugate triazole formed between the |
| | cyclooctyne group and the azide group of Z512 |
| L373Z513 | 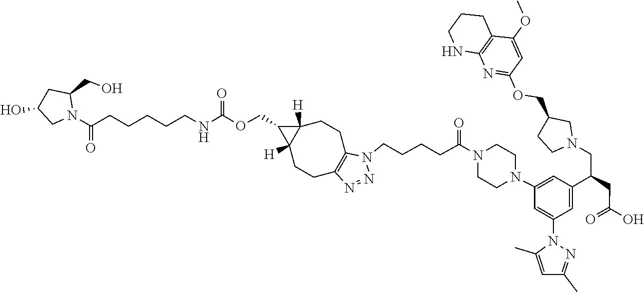 [SMILES: COc1cc(OC[C@@H]2CCN(C[C@@H](CC(=O)O)c3cc(N4CCN(C(=O)CCCCn5nnc6c5CC[C@@H]5[C@H](CC6)[C@@H]5COC(=O)NCCCCCC(=O)N5C[C@H](O)C[C@H]5CO)CC4)cc(-n4nc(C)cc4C)c3)C2)nc2c1CCCN2] |
| | ((2S,4R)-(1-(6-(((1R,8S,9S)-bicyclo[6.1.0]non-4-yn-9-ylmethoxy)carbonylamino)hexan-1- |
| | oyl)-4-hydroxypyrrolidin-2-yl)methyl)ester, click conjugate triazole formed between the |
| | cyclooctyne group and the azide group of Z513 |
| Q410s | 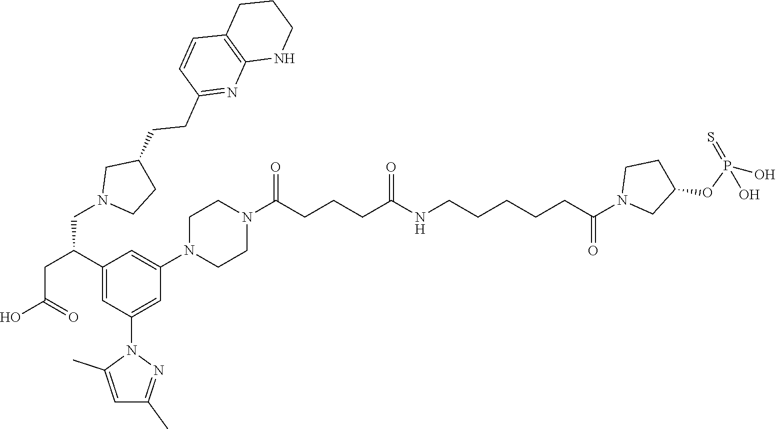 [SMILES: Cc1cc(C)n(-c2cc([C@H](CC(=O)O)CN3CC[C@@H](CCc4ccc5c(n4)NCCC5)C3)cc(N3CCN(C(=O)CCCC(=O)NCCCCCC(=O)N4CC[C@H](OP(O)(O)=S)C4)CC3)c2)n1] |
| | 1-(6-((5-(4-(3-(3,5-dimethyl-1H-pyrazol-1-yl)-5-((S)-3-carboxy-1-((R)-3-(2-(5,6,7,8- |
| | tetrahydro-1,8-naphthyridin-2-yl)ethyl)pyrrolidin-1-yl)propan-2-yl)phenyl)piperazin-1-yl)- |
| | 5-oxopentanoyl)amino)hexan-1-oyl)-3S-hydroxy-pyrrolidine 3-phosphorothioate |
| Q411s | 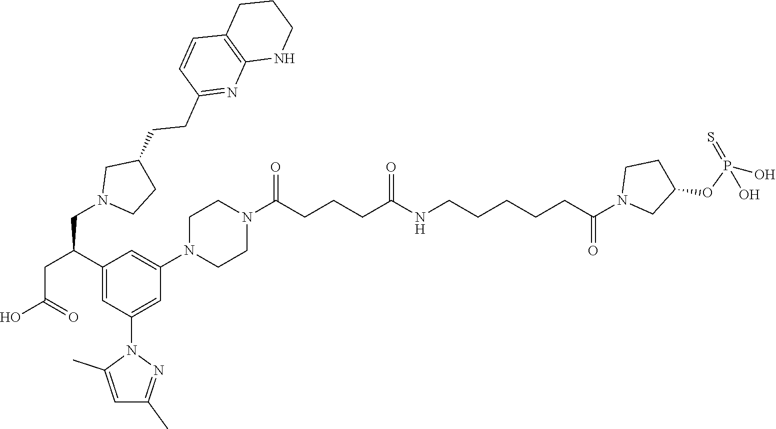 [SMILES: Cc1cc(C)n(-c2cc([C@@H](CC(=O)O)CN3CC[C@@H](CCc4ccc5c(n4)NCCC5)C3)cc(N3CCN(C(=O)CCCC(=O)NCCCCCC(=O)N4CC[C@H](OP(O)(O)=S)C4)CC3)c2)n1] |
| | 1-(6-((5-(4-(3-(3,5-dimethyl-1H-pyrazol-1-yl)-5-((R)-3-carboxy-1-((R)-3-(2-(5,6,7,8- |
| | tetrahydro-1,8-naphthyridin-2-yl)ethyl)pyrrolidin-1-yl)propan-2-yl)phenyl)piperazin-1-yl)- |
| | 5-oxopentanoyl)amino)hexan-1-oyl)-3S-hydroxy-pyrrolidine 3-phosphorothioate |
| Z80 | 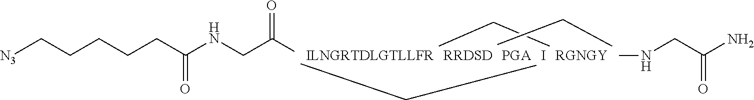 |
| | 6-azidohexan-1-oyl-GCILNGRTDLGTLLFRCRRDSDCPGACICRGNGYCG-NH₂ |
| | (SEQ ID NO: 121) where disulfide bonds exist between C2-C27, C17-C29, C23-C35 |
| Z81 | 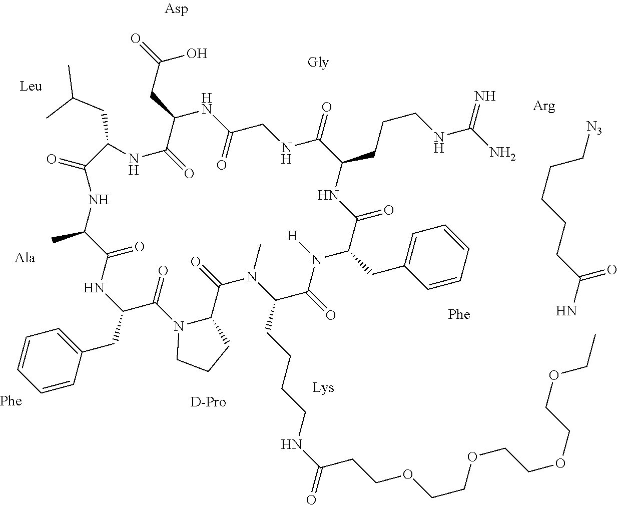 |
| | cyclo(N-MeK)GPGRGDQTTLA) wherein the lysine amino residue formes an amide with |
| | 22-azido-17-oxo-4,7,10,13-tetraoxa-16-aza-docosanoic acid (SEQ ID NO: 122) |
| Z511 | 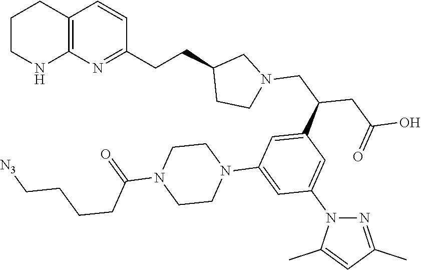 [SMILES: Cc1cc(C)n(-c2cc([C@H](CC(=O)O)CN3CC[C@@H](CCc4ccc5c(n4)NCCC5)C3)cc(N3CCN(C(=O)CCCCN=[N+]=[N-])CC3)c2)n1] |
| | (S)-3-(3-(4-(5-azidopentanoyl)piperazin-1-yl)-5-(3,5-dimethyl-1H-pyrazol-1-yl)phenyl)-4- |
| | ((R)-3-(((5,6,7,8-tetrahydro-1,8-naphthyridin-2-yl)oxy)methyl)pyrrolidin-1-yl)butanoic |
| | acid |
| Z512 | 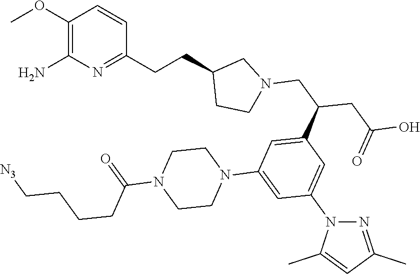 [SMILES: COc1ccc(CC[C@@H]2CCN(C[C@@H](CC(=O)O)c3cc(N4CCN(C(=O)CCCCN=[N+]=[N-])CC4)cc(-n4nc(C)cc4C)c3)C2)nc1N] |
| | (S)-4-((R)-3-(2-(6-amino-5-methoxypyridin-2-yl)ethyl)pyrrolidin-1-yl)-3-(3-(4-(5- |
| | azidopentanoyl)piperazin-1-yl)-5-(3,5-dimethyl-1H-pyrazol-1-yl)phenyl)butanoic acid |
| Z513 | 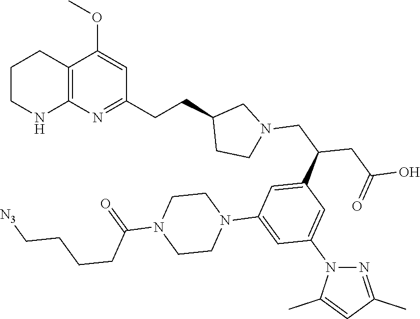 [SMILES: COc1cc(CC[C@@H]2CCN(C[C@@H](CC(=O)O)c3cc(N4CCN(C(=O)CCCCN=[N+]=[N-])CC4)cc(-n4nc(C)cc4C)c3)C2)nc2c1CCCN2] |
| | (S)-3-(3-(4-(5-azidopentanoyl)piperazin-1-yl)-5-(3,5-dimethyl-1H-pyrazol-1-yl)phenyl)-4- |
| | ((R)-3-(2-(4-methoxy-5,6,7,8-tetrahydro-1,8-naphthyridin-2-yl)ethyl)pyrrolidin-1- |
| | yl)butanoic acid |

1. A compound comprising an av 6 integrin targeting ligand, wherein the compound is of Formula (X): 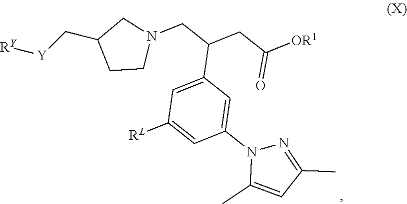
or a salt thereof, wherein:
Y is O, N(H), S, or CH₂;
R¹ is hydrogen or C₁₋₆alkyl;
R^{Y} is 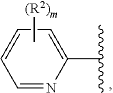
wherein
m is 0, 1, 2, 3, or 4; and
each R² is independently R, or two R² groups on adjacent carbon atoms taken together with the atoms to which they are bound form a fused 4-8 membered ring that is optionally substituted by 1, 2, 3 or 4 groups independently selected from the group consisting of R and a nitrogen protecting group;
and
R^{L} is —N(R³)(R⁴), —O(R⁵), —S(R⁵), or —R⁵, wherein
(i) R³ is hydrogen or C₁₋₆alkyl and R⁴ is R⁵; or
(ii) R³ and R⁴ taken together with the nitrogen atom to which they are attached form a 4-8 membered monocyclic heterocyclyl group that is substituted with R⁵;
and
R⁵ is -L-ZZ-L′-R^{T} wherein
L and L′ are independently -L¹-[G-L²]_{q}-G-L³-, wherein
* is the bond to ZZ;
q is 0_or an integer selected from 1-25;
L¹ is a bond or -B-A-;
each L² is independently -A-B-A-;
L³ is a bond or -A-B-A-;
each G is independently -D-E-F-, wherein D, E, and F are each independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
each A is independently a bond, —O—, —S—, or —N(R^{N})—;
each B is independently a bond, CH₂, C(O), C(S), C(NR^{N}), S(O) S(O)₂, P(O)(OH), P(S)(OH), or P(S)(SH);
each R^{N} is independently hydrogen or C₁₋₆alkyl, or two R^{N} within an -A-B-A- group taken together with the atoms to which they are connected from a 4-8 membered heterocyclyl;
and
ZZ is -A′-B′-A′- or a linking group formed by a reactive pair, wherein
each A′ is independently a bond, —O—, —S—, or —N(R^{N3})—;
each B′ is independently a bond, CH₂, C(O), C(S), C(NR^{N3}), —C═N—, S(O), S(O)₂, P(O)(OH), P(S)(OH), or P(S)(SH); and
each R^{N3} is independently hydrogen or C₁₋₆alkyl, or two R^{N3} within the -A′-B′-A′- group taken together with the atoms to which they are connected from a 4-8 membered heterocyclyl;
R^{T} is -G⁰-OR^{T1}, wherein
G⁰ is absent or -D⁰-E^{U}-F⁰-, wherein Do, E⁰, and F⁰ are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R substituents;
and
R^{T1} is hydrogen, a hydroxyl protecting group, a phosphorous coupling group, -L^{K}-S^{S}, or -L^{L}-oligonucleotide, wherein
L^{K} is a support linking group;
L^{L} is an oligonucleotide linking group; and
S^{S} is a solid support, —OR^{SS} or —N(R^{SS})₂, or hydrogen, wherein
each R^{SS} is independently hydrogen or C₁₋₆alkyl.
and
each R is independently selected from the group consisting of R′, C₁₋₆alkyl, C₁₋₆haloalkyl, C₂₋₆alkenyl, C₂₋₆alkynyl, C₃₋₈cycloalkyl, heterocyclyl, aryl, heteroaryl, C₃₋₈cycloalkylC₁₋₆alkyl, heterocyclylC₁₋₆alkyl, aryl C₁₋₆alkyl, heteroarylC₁₋₆alkyl, each of which, other than R′, is optionally substituted with 1, 2, or 3 R′ groups, wherein
each R′ is independently halogen, cyano, azido, nitro, —N(R^{b})₂, —O(R^{a}), —S(R⁰), —C(O)OR⁰,
C(O)R⁰, —C(O)N(R⁰)₂, —C(NR⁰)OR⁰, —C(NR⁰)R⁰, —C(NR⁰)N(R⁰)₂, —C(S)OR⁰, —C(S)R⁰, —C(S)N(R⁰)₂, —S(O)₂R⁰, —S(O)₂OR⁰, —S(O)₂N(R⁰)₂, —N(R⁰)C(O)OR⁰, —N(R⁰)C(O)R⁰, —N(R⁰)C(O)N(R⁰)₂, —N(R⁰)S(O)₂R⁰, —N(R⁰)S(O)₂OR⁰, —N(R⁰)S(O)₂N(R⁰)₂, —OC(O)OR⁰, —OC(O)R⁰, —OC(O)N(R⁰)₂, —OS(O)₂R⁰, —OS(O)₂OR⁰, —OS(O)₂N(R⁰)₂, or —SC(O)R⁰, wherein
each R⁰ is independently hydrogen or C₁₋₆alkyl; each R^{a} is independently hydrogen, C₁₋₆alkyl, or a hydroxyl protecting group; and each R^{b} is independently hydrogen, C₁₋₆alkyl, or a nitrogen protecting group,
provided that in each -D-E-F- group at least one of D, E, and F is not a bond; and R^{L} is not N-morpholinyl.

2. The compound of claim 1, wherein R^{Y} is; 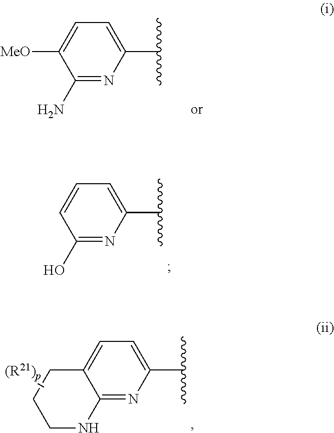
wherein p is 0, 1, 2, 3 or 4; and each R²¹ is independently selected from the group consisting of an R and a nitrogen protecting group; 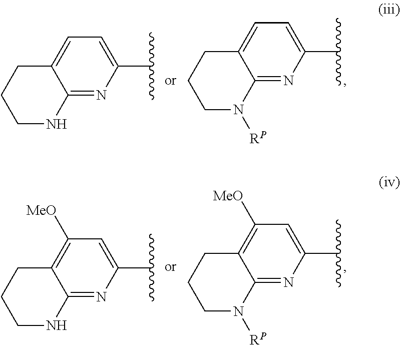
wherein R^{P} is a nitrogen protecting group; or
wherein R^{P} is a nitrogen protecting group.

3-5. (canceled)

6. The compound of claim 1, wherein -L′-R^{T} is: 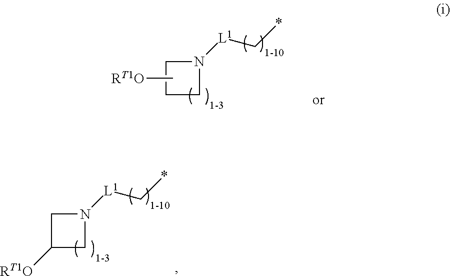
wherein * is the bond to ZZ; and L¹ is a bond, C(O), C(S), S(O)₂, P(O)(OH), or P(S)(OH), or 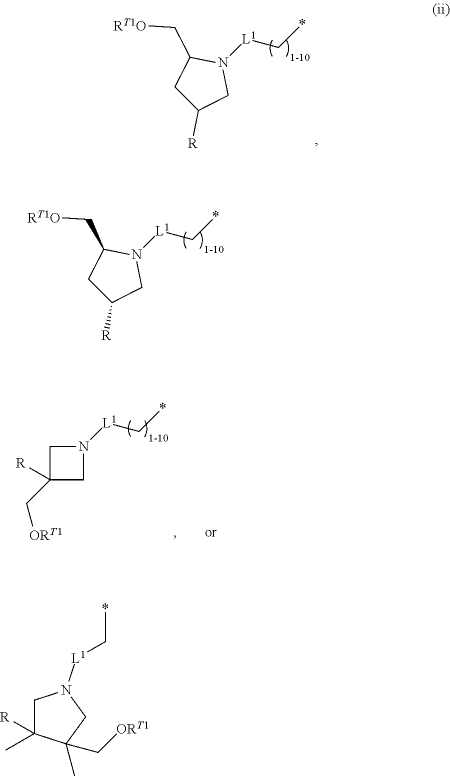
wherein* is the bond to ZZ;
L¹ is a bond, C(O), C(S), S(O)₂, P(O)(OH), or P(S)(OH); and
R is —O(R^{a}) or —C₁₋₆alkyl-O(R^{a}), wherein R^{a} is hydrogen or a hydroxyl protecting group.

7. (canceled)

8. The compound of claim 1, wherein —R^{L} is: 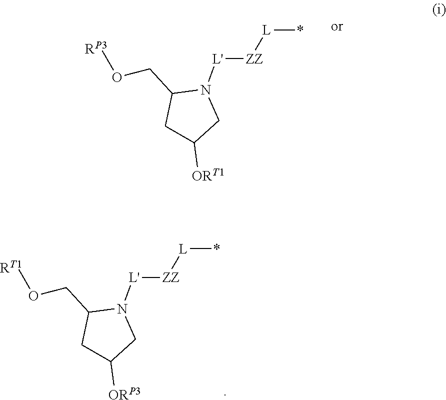
wherein R^{P3} is hydrogen or a hydroxyl protecting group; or

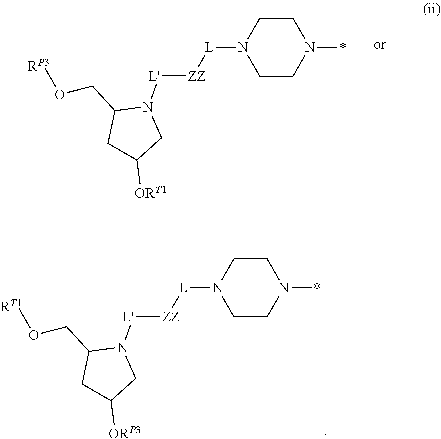

9. (canceled)

10. The compound of claim 8, wherein R^{P3} is an optionally substituted trityl group.

11. The compound of claim 1, wherein -L′- is:
(i) -L¹-G-L³-*, wherein * is the bond to ZZ; or
(ii) —C(O)—C₂₋₃₀alkyl-*.

12. (canceled)

13. The compound of claim 1 represented by the following formula:

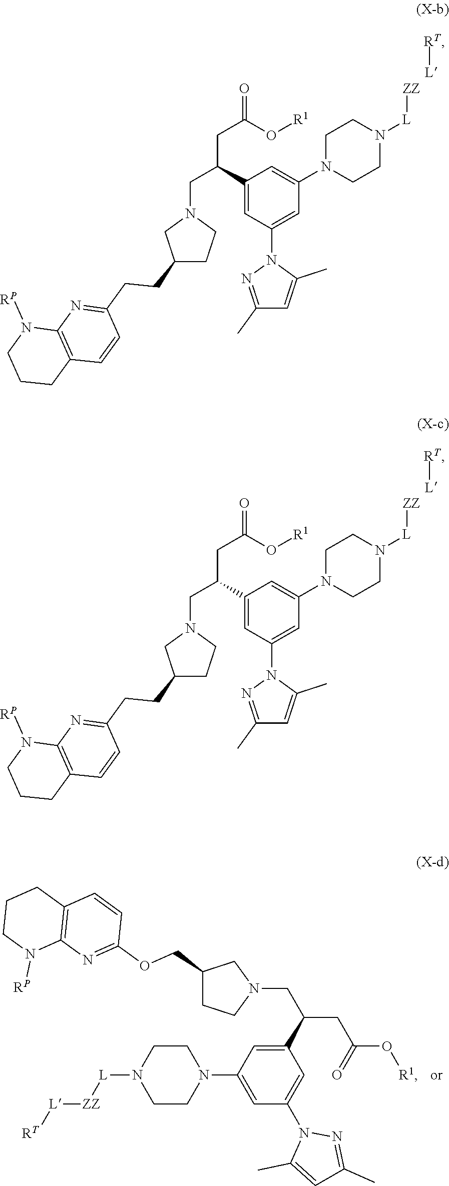

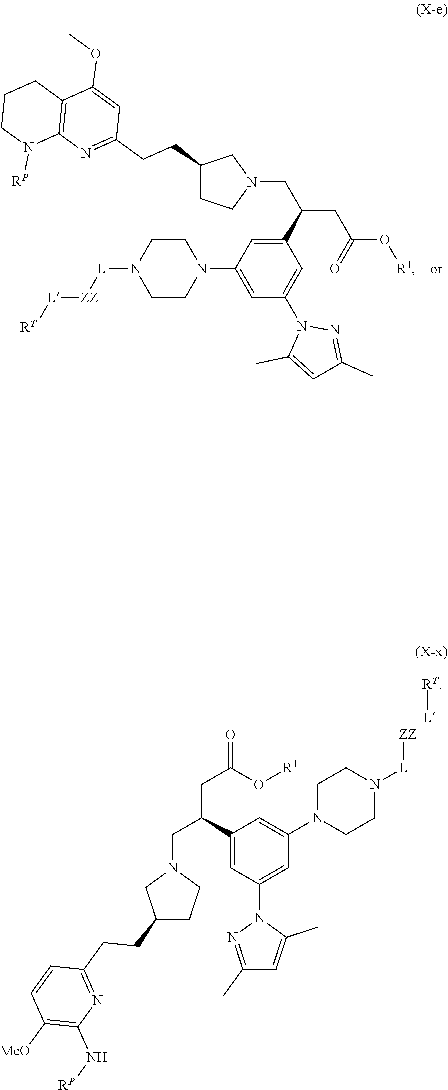

14. The compound of claim 1, wherein L is:
(i) -L¹-[G-L²]_{q}-G-L³-*, wherein q is 0, 1, 2, 3, 4, or 5;
(ii) -L¹-G-*, wherein * is the bond to ZZ;
G is C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
L¹ is -B-A-, wherein,
A is a bond, —O—, —S—, or —N(R^{N})—, wherein each R^{N} is independently hydrogen or C₁₋₆alkyl; and
B is a bond, C(O), C(S), S(O)₂, P(O)(OH), or P(S)(OH); 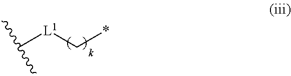
wherein * is the bond to ZZ; k is an integer from 1 to 10; L¹ is bond, C(O), C(S), C(NR^{N}), S(O)₂, P(O)(OH), or P(S)(OH); and R^{N} is hydrogen or C₁₋₆alkyl;
(iv) a group selected from: 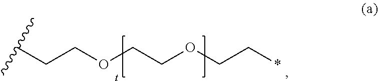
wherein * is the bond to ZZ; t is an integer from 0 to 10; 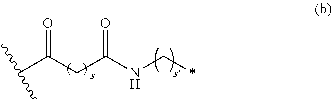
wherein * is the bond to ZZ, t is an integer from 0 to 10; a is an integer from 1 to 3 and s and s′ are each independently an integer from 1 to 24; 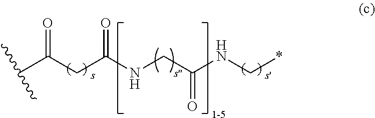
wherein * is the bond to ZZ; a is 1, 2 or 3; and each s, s′, and s″ independently is an integer from 1 to 24; 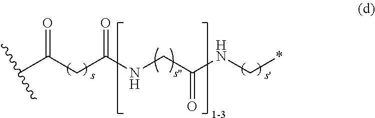
wherein * is the bond to ZZ; and s, s′, and s″ are independently is an integer from 1 to 24; 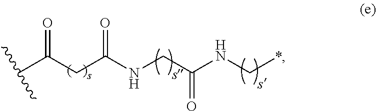
wherein * is the bond to ZZ and each s, s′, and s″ independently is an integer from 1 to 24; and 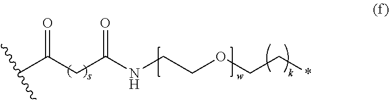
wherein * is the bond to ZZ; s and k are independently is an integer from 1 to 20; and w is an integer from 1 to 10; 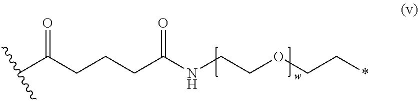
wherein * is the bond to ZZ and w is an integer from 1 to 20; or 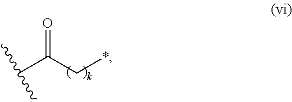
wherein * is the bond to ZZ; k is an integer from 1 to 10.

15-19. (canceled)

20. The compound of claim 1, wherein ZZ:
(i) comprises a group selected from the group consisting of: 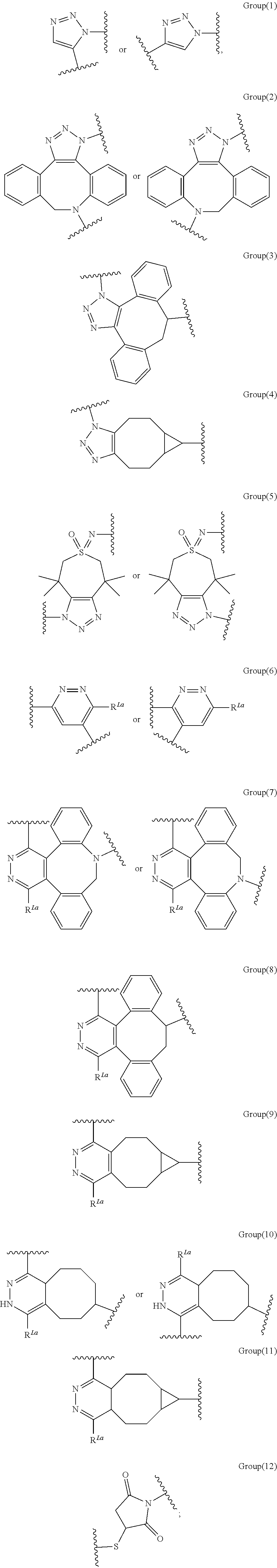
(ii) is -A′-B′-A′-, wherein
each A′ is independently a bond, —O—, —S—, or —N(R^{N3})—, wherein R^{N3} is independently hydrogen or C₁₋₆alkyl and
each B′ is independently CH₂, C(O), C(S), S(O)₂, P(O)(OH), or P(S)(OH);
(iii) is -A′-B′- or -B′-A′- wherein
each A′ is independently —O— or —N(R^{N3})—, wherein R^{N3} is independently hydrogen or C₁₋₆alkyl.
each B′ is independently CH₂, C(O), S(O)₂, P(O)(OH), or P(S)(OH); and
each R^{N3} is independently hydrogen or C₁₋₆alkyl;
(iv) is —CH₂O—, —OCH₂—, —S—S—, —C═N—, —C═N—O—, —C═N—N(R^{N3})—, —N═C—, —O—N═C—, —N(R^{N3})—N═C—, —C(O)N(R^{N3})—, —N(R^{N3})C(O)—, —C(O)O—, —OC(O)—, —OC(O)N(R^{N3})—, —N(R^{N3})C(O)O—, —N(R^{N3})C(O)N(R^{N3})—, —S(O)₂N(R^{N3})—, —N(R^{N3})S(O)₂—, —OP(O)(OH)O—, —OP(S)(OH)O—, —OP(O)(OH)—, —OP(S)(OH)—, —P(O)(OH)O—, or —P(S)(OH)O—. wherein R^{N3} is independently hydrogen or C₁₋₆alkyl;
(iv) is —C(O)N(R^{N3})— or —N(R^{N3})C(O)—, wherein R^{N3} is independently hydrogen or C₁₋₆alkyl; or
(v) is —OP(O)(OH)O—, —OP(S)(OH)O—, —OP(O)(OH)—, —OP(S)(OH)—, —P(O)(OH)O—, or —P(S)(OH)O—.

21-25. (canceled)

26. The compound of claim 1, wherein R^{T1} is -L^{L}-oligonucleotide, wherein L^{L} is a divalent linker that connects to the 3′-end of the oligonucleotide, the 5′-end of the oligonucleotide, or an internal 2′- or 3′ position on an internal nucleotide.

27. The compound of claim 26, wherein L^{L} connects to an oxygen atom on a nucleoside, and is P(O)(OH)—, —P(S)(OH)—, or —P(S)(SH).

28. The compound of claim 27, wherein L^{L} is P(O)(OH)— or P(S)(OH)—.

29. (canceled)

30. The compound of claim 1, wherein R^{T} is R^{T1}, wherein R^{T1} is -L^{L}-oligonucleotide, wherein L^{L} connects to an oxygen atom on a nucleoside of the oligonucleotide, LL is a bond, and the nucleoside is of Formula (X-f), 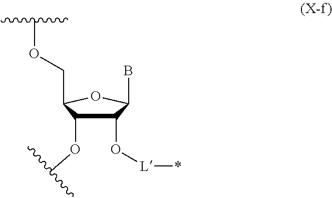
wherein B is an optionally modified nucleobase and * represents the bond to ZZ.

31. The compound of claim 30, wherein -L′- is —C₄₋₁₀alkyl-*, wherein * is the bond to ZZ, or
wherein the nucleoside is of a formula selected from the group consisting of 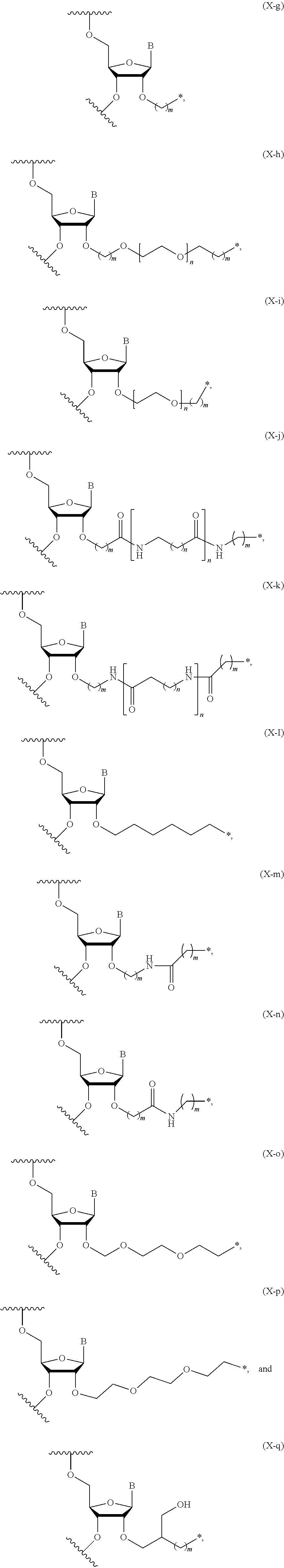
wherein
B is an optionally modified nucleobase;
each n is independently 0 or an integer selected from 1-10; and
each m is independently integer selected from 1-20.

32. (canceled)

33. The compound of claim 1, wherein R^{T1} is -L^{L}-oligonucleotide and is conjugated at the 5′-end of the oligonucleotide.

34. The compound of claim 33, represented by the following formula: 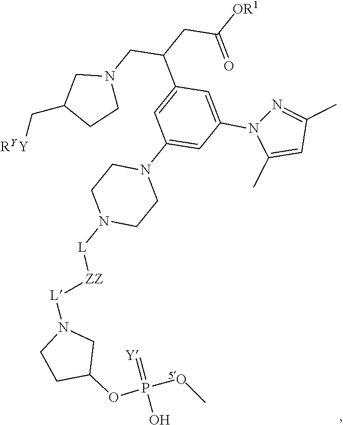
wherein Y′ is O or S.

35. The compound of claim 1, wherein R^{T1} is -L^{L}-oligonucleotide and is conjugated at the 3′-end of the oligonucleotide.

36. The compound of claim 35, having represented by the following formula:
(i) 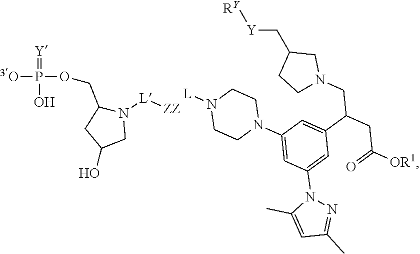
wherein Y is O or Si
(ii) 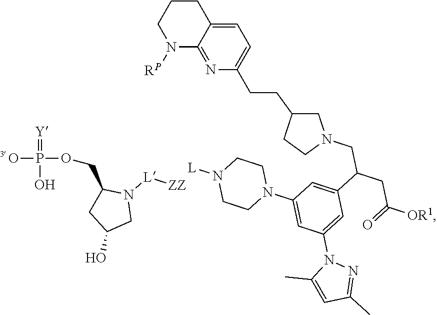
wherein Y′ is O or S, R¹ is hydrogen or C₁₋₆alkyl, and R^{P} is hydrogen or a nitrogen protecting group;
(iii) 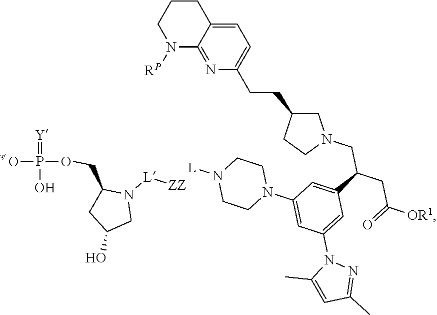
wherein Y′ is O or S, R¹ is hydrogen or C₁₋₆alkyl, and R^{P} is hydrogen or a nitrogen protecting group;
(iv) 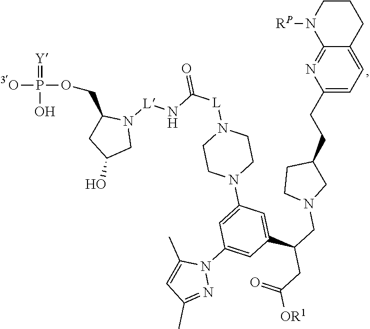
wherein Y′ is O or S, R¹ is hydrogen or C₁₋₆alkyl, and R^{P} is hydrogen or a nitrogen protecting group;
(v) 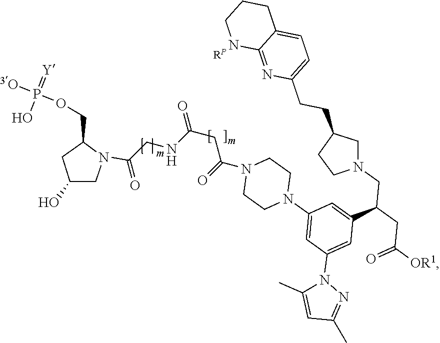
wherein each m is independently an integer selected from 1-10; Y′ is O or S, R¹ is hydrogen or C₁₋₆alkyl, and R^{P} is hydrogen or a nitrogen protecting group;
(vi) 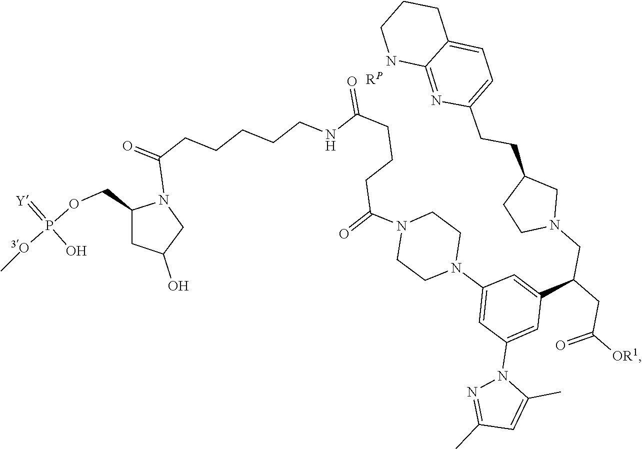
wherein Y′ is O or S, R¹ is hydrogen or C₁₋₆alkyl, and R^{P} is hydrogen or a nitrogen protecting group.

37-41. (canceled)

42. The compound of claim 36, wherein in (vi), R^{P} is hydrogen and R¹ is hydrogen; and/or wherein Y′ is O or S.

43-44. (canceled)

45. A compound comprising a αvβ6 integrin targeting ligand, wherein the compound is of Formula (XV): 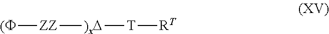
or a salt thereof, wherein
x is 2, 3, 4, 5, 6, 7, or 8;
T is a divalent linking group;
Δ is a branching group;
each ZZ is independently -A′-B′-A′- or a linking group formed by a first reactive pair,
wherein
each A′ is independently a bond, —O—, —S—, or —N(R^{N3})—;
each B′ is independently a bond, CH₂, C(O), C(S), C(NR^{N3}), —C═N—, S(O), S(O)₂, P(O)(OH), P(S)(OH), or P(S)(SH); and
each R^{N3} is independently hydrogen or C₁₋₆alkyl, or two R^{N3} within the -A′-B′-A′- group taken together with the atoms to which they are connected from a 4-8 membered heterocyclyl;
Z⁰ is a member of a second reactive pair; and
R^{T} is -G⁰-OR^{T1}, wherein
G⁰ is -D⁰-E^{U}-F⁰-, wherein
D⁰, E⁰, and F⁰ are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups; and
R^{T1} is hydrogen, a hydroxyl protecting group, a phosphorous coupling group, or -L^{K}-S^{S}, or -L^{L}-oligonucleotide, wherein
L^{K} is a support linking group;
S^{S} is a solid support, —OR^{SS} or —N(R^{SS})₂, or hydrogen, wherein
each R^{SS} is independently hydrogen or C₁₋₆alkyl; and
L^{L} is an oligonucleotide linking group; and each Φ is a compound of the Formula (XII), 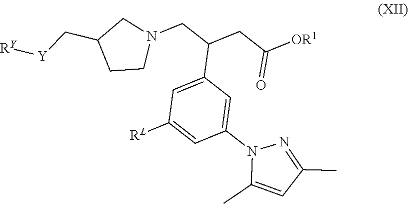
wherein:
Y is O, N(H), S, or CH₂;
R¹ is hydrogen or C₁₋₆alkyl;
R^{Y} is 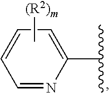
wherein
m is 0, 1, 2, 3, or 4; and
each R² is independently R, or two R² groups on adjacent carbon atoms taken together with the atoms to which they are bound form a fused 4-8 membered ring that is optionally substituted by 1, 2, 3 or 4 groups independently selected from the group consisting of R and a nitrogen protecting group;
and
R^{L} is —N(R³)(R⁴), —O(R⁵), —S(R⁵), or —R⁵, wherein
(i) R³ is hydrogen or C₁₋₆alkyl and R⁴ is R⁵; or
(ii) R³ and R⁴ taken together with the nitrogen atom to which they are attached form a 4-8 membered monocyclic heterocyclyl group that is substituted with R⁵;
and
R⁵ is -L-* wherein L is -L¹-[G-L²]_{q}-G-L³-*,
* is the bond to a ZZ; and
q is 0 or an integer selected from 1-25;
L¹ is a bond or -B-A-;
each L² is independently -A-B-A-;
L³ is a bond or -A-B-A-;
each G is independently -D-E-F-, wherein
D, E, and F are independently a bond, C₁₋₁₀alkyl, C₂₋₁₀alkenyl, C₂₋₁₀alkynyl, C₃₋₁₀cycloalkyl, 3-10 membered heterocyclyl, aryl, or heteroaryl, each of which is optionally substituted with 1, 2, 3, or 4 R groups;
each A is independently a bond, —O—, —S—, or —N(R^{N})—;
each B is independently a bond, CH₂, C(O), C(S), C(NR^{N}), S(O), S(O)₂, P(O)(OH), P(S)(OH), or P(S)(SH);
each R^{N} is independently hydrogen or C₁₋₆alkyl, or two R^{N} within an -A-B-A- group taken together with the atoms to which they are connected from a 4-8 membered heterocyclyl.

46-109. (canceled)

110. A dsRNA agent for inhibiting the expression of a target gene, comprising the compound comprising an αvβ6 integrin targeting ligand of claim 1.

111. The dsRNA agent of claim 110, wherein the αvβ6 integrin targeting ligand is conjugated to the sense strand.

112. The dsRNA agent of claim 111, wherein the αvβ6 integrin targeting ligand;
(i) is conjugated to the 3′-end of the sense strand;
(ii) is conjugated to the 5′-end of the sense strand;
(iii) is conjugated to both the 5′-end and the 3′-end of the sense strand; or
(iv) is conjugated to the an internal position of the sense strand.

113-115. (canceled)

116. The dsRNA agent of claim 110, wherein the αvβ6 integrin targeting ligand is conjugated to the antisense strand.

117. The dsRNA agent of claim 116, wherein the αvβ6 integrin targeting ligand;
(i) is conjugated to the 3′ end antisense strand; or
(ii) is conjugated to an internal position of antisense strand.

118. (canceled)

119. The dsRNA agent of claim 110, wherein the target gene is selected from the group consisting of adrenoceptor beta 1 (ADRB1); calcium voltage-gated channel subunit alpha1 C (CACNA1C); calcium voltage-gated channel subunit alpha1 G (CACNA1G) (T type calcium cchannel); angiotensin II receptor type 1 (AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); and Phosphodiesterase 1 (PDE1); myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); Double Homeobox 4 (DUX4); dystrophy myotonic protein kinase (DMPK); glycogen synthase 1 (GYS1); survival of motor neuron 1 (SMN1), and alpha-glucosidase (GAA).

120. A cell containing the dsRNA agent of claim 110.

121. A pharmaceutical composition for inhibiting expression of the target gene, comprising the dsRNA agent of claim 110.

122. A method of inhibiting expression of a target gene in a skeletal muscle cell and/or a cardiac muscle cell, comprising contacting the cell with the dsRNA agent of claim 110, thereby inhibiting expression of the target gene in the skeletal muscle cell and/or the cardiac muscle cell.

123. The method of claim 122, wherein the cell is within a subject.

124. The method of claim 123, wherein the subject is a human.

125. A method of treating a subject having a skeletal muscle disorder and/or a cardiac muscle disorder, comprising administering to the subject a therapeutically effective amount of the dsRNA agent of claim 110, thereby treating the subject.

126. The method of claim 125, wherein the skeletal muscle disorder and/or cardiac muscle disorder is selected from the group consisting of myostatin-related muscle hypertrophy, congenital myasthenic syndrome, facioscapulohumeral muscular dystrophy (FSHD), Spinal Muscular Atrophy (SMA), Myotonic Dystrophy Type 1 (DM1), Pompe disease, PLN cardiomyopathy, spasticity, obstructive hypertrophic cardiomyopathy (HOCM); familial hypertrophic cardiomyopathy (FHC); heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); hypertrophic cardiomyopathy (HCM), dilated cardiomyopathy (DCM), arrhythmia, and congestive heart failure (CHF).

127. A method of inhibiting expression of a target gene in a lung cell, comprising contacting the cell with the dsRNA agent of claim 110, thereby inhibiting expression of the target gene in the lung cell.

128. The method of claim 127, wherein the cell is within a subject.

129. The method of claim 128, wherein the subject is a human.

130. A method of treating a subject having a lung disorder, comprising administering to the subject a therapeutically effective amount of the dsRNA agent of claim 110, thereby treating the subject.

131. The method of claim 130, wherein the lung disorder is selected from the group consisting of idiopathic pulmonary fibrosis, asthma, asthma and chronic rhinosinusitis, nasal polyps and chronic rhinosinusitis.

132. The method of claim 122, wherein the dsRNA agent is administered to the subject subcutaneously, intramuscularly, intravenously or via inhalation.

133-135. (canceled)

136. An RNA-induced silencing complex (RISC) comprising an antisense strand of any of the dsRNA agents of claim 110.
